(19) United States
(12) Patent Application Publication
(10) Pub. No.: US 2023/0146455 A1
(43) Pub. Date: May 11, 2023

(54) TREATMENT OF INFLAMMATORY DISEASES WITH PEPTIDES AND PHARMACEUTICAL COMPOSITIONS

(71) Applicant: Yuyu Pharma, Inc., Seoul (KR)

(72) Inventors: Wonsang Yu, Seoul (KR); Larry Chong Park, Chungbuk (KR)

(21) Appl. No.: 17/908,396

(22) PCT Filed: Mar. 5, 2021
(86) PCT No.: PCT/IB21/00423

§ 371 Date: Aug. 31, 2022

Related U.S. Application Data
(60) Provisional application No. 62/985,603, filed on Mar. 5, 2020.

(51) Int. Cl. A61K 38/08; A61P 19/02; A61P 19/10; A61P 37/06; A61P 27/02

(52) U.S. Cl. CPC A61K 38/08; A61P 19/02; A61P 19/10; A61P 37/06; A61P 27/02

(57) ABSTRACT

Disclosed are methods of treating inflammatory diseases with peptides and pharmaceutical compositions using peptides.

### CROSS-REFERENCE TO A RELATED APPLICATION

This application claims the benefit of U.S. Provisional Application No. 62/985,603, filed on Mar. 5, 2020, which application is hereby incorporated by reference in its entirety.

### TECHNICAL FIELD

The present invention relates to treating inflammatory diseases with peptides and pharmaceutical compositions.

### BACKGROUND

Inflammation is a protective mechanism in mammals from invading pathogens. However uncontrolled inflammation can cause tissue damage and is the cause of many diseases. Inflammatory diseases, either chronic or acute in nature, afflict many patients every day and present an important problem in the health care industry. Diseases and disorders which have significant inflammatory components include skin disorders, bowel disorders, certain degenerative neurological disorders, arthritis, and autoimmune diseases. For some patients, dietary or environmental factors may trigger an autoimmune or inflammatory response. In other patients, genetic factors can play a key part in disease. In many inflammatory conditions, pro-inflammatory cytokines, particularly IL-1β, and IL-6, play an important role in the pathogenesis of various inflammation-related diseases.

Interleukin-6 (IL-6) is a major pro-inflammatory cytokine and consists of 212 amino acids with two N-linked glycosylation sites. The IL-6 glycoprotein has a molecular weight of about 26 kDa. IL-6 signaling is mediated by the binding of IL-6 to either soluble or surface bound IL-6 receptor chain (IL-6R), enabling interaction of the complex with the cell surface transmembrane gp130 subunit. The interaction mediates intracellular signaling and is responsible for the proliferation and differentiation of immune cells.

Interleukin-1 beta (IL-1β) is a pro-inflammatory cytokine that is produced as a precursor by activated macrophages. The molecular weight of the proteolytically processed IL-1β is 17.5 kDa. Upon proteolytic cleavage, signal transduction is initiated by binding of active IL-1β to IL-1 receptor type I (IL-1R1), which in turn associates with the transmembrane IL-1 receptor accessory protein (IL-1RAP). The formed complex triggers signal transduction. IL-1β is key mediator in the inflammatory response and the cytokine affects a number of cellular activities such as cell proliferation, differentiation, and apoptosis.

New therapies for reducing inflammation are needed.

### SUMMARY OF INVENTION

In one aspect, provided herein are methods of preventing or treating an IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine mediated disease or disorder in a subject, comprising administering to the subject a compound disclosed herein.

In a further aspect, provided are methods of reducing production of IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine in cells of a subject, comprising administering to the subject a compound disclosed herein.

In yet another aspect, provided are methods of reducing NF-κB transcription activity in cells of a subject, comprising administering to the subject a compound disclosed herein.

In another aspect, provided are methods of preventing or treating Keratoconjunctivitis sicca (Dry Eye) in a subject, comprising administering to the subject a compound disclosed herein.

In yet another aspect, provided are methods of preventing or treating Dry Eye Disease (DED) in a subject, comprising administering to the subject a compound disclosed herein.

### BRIEF DESCRIPTION OF THE DRAWINGS

FIG. 1A-1D are diagrams showing attenuation of IL-6 release of poly I:C stimulated primary human corneal epithelial cells by test compounds in a dose dependent manner; Compounds tested: YDE-093, YDE-096, YDE-100, YDE-101, YDE-102, YDE-103, YDE-104, YDE-105, YDE-106, and YDE-107.

FIG. 2A-2BF are diagrams showing an effect on cell proliferation of primary corneal epithelial cells after 48 hrs and 72 hrs with the following test compounds: YDE-012, YDE-019, YDE-038, YDE-044, YDE-045, YDE-047, YDE-048, YDE-049, YDE-050, YDE-051, YDE-052, YDE-053, YDE-054, YDE-055, YDE-056, YDE-057, YDE-058, YDE-059, YDE-060, YDE-061, YDE-062, YDE-063, YDE-064, YDE-065, YDE-066, YDE-067, YDE-072, YDE-073, YDE-074, YDE-075, YDE-076, YDE-077, YDE-078, YDE-079, YDE-080, YDE-081, YDE-082, YDE-083, YDE-084, YDE-085, YDE-086, YDE-087, Diquas, and hEGF.

FIG. 3 is a diagram showing the effect of YDE-053, YDE-060, or YDE-065 on the IL-1beta, IL-6, IL-8, MIP-1 alpha, MIP-1 beta, RANTES, and TNF-alpha release of poly I:C stimulated primary human corneal epithelial cells.

FIG. 4 is a diagram showing the effect of YDE-053 on NF-κB (p65) transcription activity of poly I:C stimulated primary human corneal cells.

FIG. 5 is a diagram representing the effect of test compounds on IL-6 release of poly I:C stimulated primary human corneal epithelial cells; Compounds tested: YDE-053, YDE-048, YDE-056, YDE-057, YDE-058, YDE-067, YDE-079, YDE-011, YDE-093, YDE-096, YDE-053, and YDE-043.

FIG. 6 is a diagram showing the effect of various stimulants on the level of IL-6.

FIG. 7 is a diagram showing the effect of Xiidra® on the level of IL-6 induced by various stimulants.

FIG. 8 is a diagram showing the effect of YDE-011 on the level of IL-6 induced by various stimulants.

FIG. 9 is a diagram showing the effect of YY-101, YDE-011, and YDE-043 on the level of IL-2 induced by LPS.

FIG. 10 is a diagram showing the effect of YY-101, YDE-011, and YDE-043 on the level of IL-6 induced by LPS.

FIG. 11 is a diagram showing the effect of YY-101, YDE-011, and YDE-043 on the level of IL-8 induced by LPS.

FIG. 12 is a diagram showing the effect of YY-101, YDE-011, and YDE-043 on the level of IL-10 induced by LPS.

FIG. 13 is a diagram showing the effect of YY-101, YDE-011, and YDE-043 on the level of TNF-α induced by LPS.

FIG. 14 is a diagram showing the effect of YY-101, YDE-011, and YDE-043 on the level of MMP-3 induced by LPS.

FIG. 15 is a diagram showing the effect of YY-101, YDE-011, and YDE-043 on the level of CCL2/MCP-1 induced by LPS.

FIG. 16 is a diagram showing the effect of YY-101, YDE-011, and YDE-043 on the level of CCL3/MIP-1α induced by LPS.

FIG. 17 is a diagram showing the effect of YY-101, YDE-011, and YDE-043 on the level of CCL4/MIP-1β induced by LPS.

FIG. 18 is a diagram showing the effect of YY-101, YDE-011, and YDE-043 on the level of IL-1α/IL-1F1 induced by LPS.

FIG. 19 is a diagram showing the effect of YY-101, YDE-011, and YDE-043 on the level of IL-1β/IL-1F2 induced by LPS.

FIG. 20 is a diagram showing the effect of YY-101, YDE-011, and YDE-043 on the level of TIMP-1 induced by LPS.

FIG. 21 is a diagram showing the effect of YY-101, YDE-011, and YDE-043 on the level of Leptin induced by LPS.

FIG. 22 is a diagram showing the effect of YY-101, YDE-011, and YDE-043 on the level of MMP-9 induced by LPS.

FIG. 23 is a diagram showing the effect of YY-101, YDE-011, and YDE-043 on the level of IL-10 induced by LPS.

FIG. 24 is a diagram showing the effect of YY-101, YDE-011, and YDE-043 on the level of IL-8 induced by LPS.

FIG. 25 is a diagram showing the effect of YY-101, YDE-011, and YDE-043 on the level of IL-6 induced by LPS.

FIG. 26 is a diagram showing the effect of YY-101, YDE-011, and YDE-043 on the level of TNF-α induced by LPS.

FIG. 27 is a diagram showing the effect of YY-101, YDE-011, and YDE-043 on the level of CCL3/MIP-1α induced by LPS.

FIG. 28 is a diagram showing the effect of YY-101, YDE-011, and YDE-043 on the level of CCL4/MIP-1β induced by LPS.

FIG. 29 is a diagram showing the effect of YY-101, YDE-011, and YDE-043 on the level of CCL5/RANTES induced by LPS.

FIG. 30 is a diagram showing the effect of YY-101, YDE-011, and YDE-043 on the level of IFN-γ induced by LPS.

FIG. 31 is a diagram showing the effect of YY-101, YDE-011, and YDE-043 on the level of CCL2/MCP-1 induced by LPS.

FIG. 32 is a diagram showing the effect of YY-101, YDE-011, and YDE-043 on the level of Fas Ligand induced by LPS.

FIG. 33 is a diagram showing the effect of YY-101, YDE-011, and YDE-043 on the level of CCL20/MIP-3a induced by LPS.

FIG. 34 is a diagram showing the effect of YY-101, YDE-011, and YDE-043 on the level of IL-1α induced by LPS.

FIG. 35 is a diagram showing the effect of YY-101, YDE-011, and YDE-043 on the level of IL-1β/IL-1F2 induced by LPS.

FIG. 36 is a diagram showing the effect of YY-101, YDE-011, and YDE-043 on the level of TIMP-1 induced by LPS.

FIG. 37 is a diagram showing the effect of YY-101, YDE-011, and YDE-043 on the level of GM-CSF induced by LPS.

FIG. 38 is a diagram showing the effect of YY-101, YDE-011, and YDE-043 on the level of IL-2 induced by LPS.

FIG. 39 is a diagram showing the effect of YY-101, YDE-011, and YDE-043 on the level of IL-17A induced by LPS.

FIG. 40 is a diagram showing the effect of YY-101, YDE-011, and YDE-043 on the level of IL-4 induced by LPS.

FIG. 41 is a diagram showing the effect of YY-101, YDE-011, and YDE-043 on the level of VEGF-A induced by LPS.

FIG. 42 is a diagram showing the effect of YY-101, YDE-011, and YDE-043 on the level of MMP-9 induced by LPS.

FIG. 43 is a diagram showing the effect of YY-101, YDE-011, YDE-043, YDE-048, and YDE-060 on the level of Eotaxin & Gro-α_KC in rat tear.

FIG. 44 is a diagram showing the effect of YY-101, YDE-011, YDE-043, YDE-048, and YDE-060 on the level of IL-17A & IL-1β in rat tear.

FIG. 45 is a diagram showing the effect of YY-101, YDE-011, YDE-043, YDE-048, and YDE-060 on the level of IL-21 & IL-4 in rat tear.

FIG. 46 is a diagram showing the effect of YY-101, YDE-011, YDE-043, YDE-048, and YDE-060 on the level of MCP-1 & MCP-3 in rat tear.

FIG. 47 is a diagram showing the effect of YY-101, YDE-011, YDE-043, YDE-048, and YDE-060 on the level of sVCAM-1 & TNF-α in rat tear.

FIG. 48 is a diagram showing the effect of YY-101, YDE-011, YDE-043, YDE-048, and YDE-060 on the level of IL-12p70 & VEGF-A in rat tear.

FIG. 49 is a diagram showing the effect of YY-101, YDE-011, YDE-043, YDE-048, and YDE-060 on the level of IL-1α & IP-10 in rat tear.

FIG. 50 is a diagram showing the effect of YY-101, YDE-011, YDE-043, YDE-048, and YDE-060 on the level of bNGF & Leptin in rat tear.

FIG. 51 is a diagram showing the effect of YY-101, YDE-011, YDE-043, YDE-048, and YDE-060 on the level of RANTES in rat tear.

FIG. 52 is a diagram showing the effect of YDE-048 and YDE-043 on the level of Gro-α_KC & IL-1α in rat goblet cell.

FIG. 53 is a diagram showing the effect of YDE-048 and YDE-043 on the level of IL-1β & Leptin in rat goblet cell.

FIG. 54 is a diagram showing the effect of YDE-048 and YDE-043 on the level of IP-10 & VEGF-A in rat goblet cell.

FIG. 55 is a diagram showing the effect of YDE-048 and YDE-043 on the level of Eotaxin & IL-17A in rat goblet cell.

FIG. 56 is a diagram showing the effect of YDE-048 and YDE-043 on the level of MCP-1 & MCP-3 in rat goblet cell.

FIG. 57 is a diagram showing the effect of YDE-048 and YDE-043 on the level of RANTES & sVCAM-1 in rat goblet cell.

FIG. 58 is a diagram showing the effect of YDE-048 and YDE-043 on the level of TGF-β & TNF-α in rat goblet cell.

FIG. 59 is a diagram showing the effect of YDE-048 and YDE-043 on the level of IL-12p70, IL-21, bNGF in rat goblet cell.

FIG. 60 is a diagram showing the effect of YDE-048 and YDE-043 on the level of IL-4 in rat goblet cell.

FIG. 61 is a diagram showing the effect of YDE-048 and YDE-043 on the level of IL-6 in rat goblet cell.

FIG. 62 shows an experimental schematic timeline of Mouse IBD Model in Example 12.

FIG. 63 shows body weight & BW change over time, disease activity index. a: found dead (G4; n=3); b: animals sacrificed moribund (G2; n=3, G3; n=1, G4; n=1, G5; n=1); a: found dead (G4; n=3); b: animals sacrificed moribund (G2; n=3, G3; n=1, G4; n=1, G5; n=1).

FIG. 64 shows clinical scores (individual scores of DAI). a: found dead (G4; n=3); b: animals sacrificed moribund (G2; n=3, G3; n=1, G4; n=1, G5; n=1); All values were presented as mean±standard error (SEM); All values were statistically analyzed by one-way ANOVA with LSD post-hoc analysis; *: 0.05>p, **: 0.01>p, ***: 0.01>p; G2 vs G3, G4, G5.

FIG. 65 shows CRP Levels (C-Reactive Protein) from Plasma. G1; n=8, G2; n=8, G3; n=8, G4; n=5, G5; n=8. Statistical analysis. All values were presented as mean±standard error (SEM). All values were statistically analyzed by one-way ANOVA with LSD post-hoc analysis. *: 0.05>p, **: 0.01>p, ***: 0.001>p; G2 vs G3, G4, G5. #: 0.05>p, ##: 0.01>p, ###: 0.001>p; G1 vs G2, G3, G4, G5 FIG. 66 shows study design of Mouse CIA-induced Rheumatoid Arthritis Model.

FIG. 67 shows Body weight & Hind Paw Thickness (Inflammation).

FIG. 68 shows overall clinical symptom & score. Clinical score: 0—no change; 1—swelling and erythema of the digit; 2—mild swelling and erythema of the limb; 3—gross swelling and erythema of the digit; 4—gross deformity and inability to use the limb.

FIG. 69 shows a study scheme & group information of OVX induced Osteoporosis disease model in C57bL6 mouse.

FIG. 70 shows Body weight and Uterus Weight & Gross Observation. G1; n=7, G2; n=7, G3; n=7, G4; n=7. Statistical analysis: All values were presented as mean±standard error (SEM). All values were statistically analyzed by one-way ANOVA with LSD post-hoc analysis. *: 0.05>p, **: 0.01>p, ***: 0.001>p; G1 vs G2, G3, G4. #: 0.05>p, ##: 0.01>p, ###: 0.001>p; G2 vs G3, G4.

FIG. 71 shows results of Lumbar Vertebrae Bone Strength Test. 0.75 kgf/1 cm; 0.75*9.8 N*x cm=Force (N*cm). Top-right image shows device used for compress test. Middle-right image shows total weight. Bottom-right image shows sample before test.

FIG. 72 shows Micro CT Parameters.

FIG. 73 shows results of Micro CT: Trabecular Area Analysis. BMD: Bone Mineral Density; Tb.N: Trabecular Number; Tb.Th: Trabecular Thickness; Tb. Sp: Trabecular Separation; Conn.D: Connectivity density. TV: Total volume; BV: Trabecular Bone Volume; BS: Bone Separation; BV/TV: Bone Volume/Total Volume; BS/TV: Bone Separation/Total Volume; BS/BV: Bone Separation/Bone Volume. G1; n=7, G2; n=7, G3; n=7, G4; n=7. Statistical analysis: All values were presented as mean±standard error (SEM). All values were statistically analyzed by one-way ANOVA with LSD post-hoc analysis. *: 0.05>p, **: 0.01>p, ***: 0.001>p; G1 vs G2, G3, G4. #: 0.05>p, ##: 0.01>p, ###: 0.001>p; G2 vs G3, G4.

FIG. 74 shows results of Micro CT: Cortical Area Analysis. BMD: Bone Mineral Density. BV: Cortical Bone Volume. Tt/Ar: Total cross-sectional area inside the periosteal envelope. Ct.Ar: Cortical bone area. Ma.Ar: Medullary (or marrow) area. Ct.Ar/Tt.Ar: Cortical area fraction. Ct.Th: Average cortical thickness. Ps.Pm: Periosteal perimeter. Ec.Pm: Endocortical perimeter. G1; n=7, G2; n=7, G3; n=7, G4; n=7. Statistical analysis. All values were presented as mean±standard error (SEM). All values were statistically analyzed by one-way ANOVA with LSD post-hoc analysis. *: 0.05>p, **: 0.01>p, ***: 0.001>p; G1 vs G2, G3, G4. #: 0.05>p, ##: 0.01>p, ###: 0.001>p; G2 vs G3, G4.

FIG. 75 shows Micro CT: Femur 2D Images.

FIG. 76 shows Micro CT: Trabecular 3D Images.

FIG. 77 shows exemplary micrographs of inflammation and oxidative markers.

FIG. 78 shows an exemplary graph of TNF-α measurement. Statistical analysis: All values were presented as mean±standard error (SEM). All values were statistically analyzed by one-way ANOVA with LSD post-hoc analysis. *: 0.05>p, **: 0.01>p, ***: 0.001>p; G1 vs All group. #: 0.05>p, ##: 0.01>p, ###: 0.001>p; G2 vs G3, G4, G5, G6.

FIG. 79 shows exemplary nitrotyrosine histology stains.

### DETAILED DESCRIPTION OF THE INVENTION

### Definitions

According to the convention used in the art,

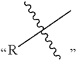

in the formulae herein is used to indicate that a moiety or substituent “R” is attached to a backbone structure.

“Alkyl” is a hydrocarbon having primary, secondary, tertiary, and/or quaternary carbon atoms, and encompasses straight, branched, and cyclic groups, or a combination thereof. For example, an alkyl group may have 1 to 20 carbon atoms (i.e., C₁-C₂₀ alkyl), 1 to 10 carbon atoms (i.e., C₁-C₁₀ alkyl), or 1 to 6 carbon atoms (i.e., C₁-C₆ alkyl). Examples of a suitable alkyl group include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), and octyl (—(CH₂)₇CH₃), but it is not limited thereto.

“Alkoxy” refers to a group having the formula —O-alkyl, wherein the alkyl group as defined above is attached to the parent compound via an oxygen atom. The alkyl moiety of the alkoxy group may have, for example, 1 to 20 carbon atoms (i.e., C₁-C₂₀ alkoxy), 1 to 12 carbon atoms (i.e., C₁-C₁₂ alkoxy), 1 to 10 carbon atoms (i.e., C₁-C₁₀ alkoxy), or 1 to 6 carbon atoms (i.e., C₁-C₆ alkoxy). Examples of a suitable alkoxy group include methoxy (—O—CH₃ or —OMe), ethoxy (—OCH₂CH₃ or —OEt), and t-butoxy (—OC(CH₃)₃ or —O-tBu), but it is not limited thereto.

“Haloalkyl” is an alkyl group in which at least one of the hydrogen atoms of the alkyl group as defined above is replaced by a halogen atom. The alkyl moiety of the haloalkyl group may have 1 to 20 carbon atoms (i.e., C₁-C₂₀ haloalkyl), 1 to 12 carbon atoms (i.e., C₁-C₁₂ haloalkyl), 1 to 10 carbon atoms (i.e., C₁-C₁₀ haloalkyl), or 1 to 6 carbon atoms (i.e., C₁-C₆ haloalkyl). Examples of a suitable haloalkyl group include —CF₃, —CHF₂, —CFH₂, and —CH₂CF₃, but it is not limited thereto.

“Alkenyl” is a hydrocarbon having primary, secondary, tertiary, and/or quaternary carbon atoms, and encompasses straight, branched, and cyclic groups, or a combination thereof, and having at least one unsaturated region, i.e., a carbon-carbon sp² double bond. For example, an alkenyl group may have 2 to 20 carbon atoms (i.e., C₂-C₂₀ alkenyl), 2 to 12 carbon atoms (i.e., C₂-C₁₂ alkenyl), 2 to 10 carbon atoms (i.e., C₂-C₁₀ alkenyl), or 2 to 6 carbon atoms (i.e., C₂-C₆ alkenyl). Examples of a suitable alkenyl group include vinyl (—CH═CH₂), allyl (—CH₂CH═CH₂), cyclopentenyl (—C₅H₇), and 5-hexenyl (—CH₂CH₂CH₂CH₂CH═CH₂), but it is not limited thereto.

“Alkynyl” is a hydrocarbon having primary, secondary, tertiary, and/or quaternary carbon atoms, and encompasses straight, branched, and cyclic groups, or a combination thereof, and having at least one carbon-carbon sp triple bond. For example, an alkynyl group may have 2 to 20 carbon atoms (i.e., C₂-C₂₀ alkynyl), 2 to 12 carbon atoms (i.e., C₂-C₁₂ alkynyl), 2 to 10 carbon atoms (i.e., C₂-C₁₀ alkynyl), or 2 to 6 carbon atoms (i.e., C₂-C₆ alkynyl). Examples of a suitable alkenyl group include acetylenic (—C≡CH) and propargyl (—CH₂C≡CH), but it is not limited thereto.

“Alkylene” refers to a saturated hydrocarbon group that may be branched, straight, or cyclic (or may have a combination of branched, straight, or cyclic moeities) and has two valencies derived by a removal of two hydrogen atoms from the same carbon atom or two different carbon atoms of a parent alkane. For example, an alkylene group may have 1 to 20 carbon atoms, 1 to 10 carbon atoms, or 1 to 6 carbon atoms. Examples of a typical alkylene group include 1,2-ethylene (—CH₂—CH₂—), but it is not limited thereto.

“Alkenylene” refers to an unsaturated hydrocarbon group that may be branched, straight, or cyclic (or may have a combination of branched, straight, or cyclic moeities) and has two valencies derived by a removal of two hydrogen atoms from the same carbon atom or two different carbon atoms of a parent alkene. For example, an alkenylene group may have 1 to 20 carbon atoms, 1 to 10 carbon atoms, or 1 to 6 carbon atoms. Examples of a typical alkenylene group include 1,2-ethenylene (—CH═CH—), but it is not limited thereto.

“Alkynylene” refers to an unsaturated hydrocarbon group that may be branched, straight, or cyclic (or may have a combination of branched, straight, or cyclic moeities) and has two valencies derived by a removal of two hydrogen atoms from the same carbon atom or two different carbon atoms of a parent alkyne. For example, an alkynylene group may have 1 to 20 carbon atoms, 1 to 10 carbon atoms, or 1 to carbon atoms. Examples of a typical alkynylene radical include acetylenylene (—C≡C), propargylene (—CH₂C≡C—), and 4-pentynylene (—CH₂CH₂CH₂C≡C—), but it is not limited thereto.

“Aryl” refers to an aromatic hydrocarbon group. For example, an aryl group may have 6 to 20 carbon atoms, 6 to 14 carbon atoms, or 6 to 12 carbon atoms. Examples of a typical aryl group include a radical derived from benzene (e.g., phenyl), substituted benzene, substituted or unsubstituted naphthalene, substituted or unsubstituted anthracene, and the like, but it is not limited thereto.

“Arylalkyl” refers to an acyclic alkyl group in which one hydrogen atom bonded to a carbon atom, typically a terminal or other sp³ carbon atom, is replaced by an aryl group. Examples of a typical arylalkyl group include benzyl, 2-phenylethan-1-yl, naphthylmethyl, 2-naphthylethan-1-yl, naphthobenzyl, 2-naphthophenylethan-1-yl, and the like (each of which may be substituted or unsubstituted), but it is not limited thereto. An arylalkyl group may have 7 to 20 carbon atoms. For example, the alkyl moiety thereof may have 1 to 6 carbon atoms, and the aryl moiety thereof may have 6 to 14 carbon atoms.

“Arylalkenyl” refers to an acyclic alkenyl group in which one of the hydrogen atoms bonded to a carbon atom, typically a terminal or other sp³ carbon atom, although an sp² carbon atom may also be used, is replaced by an aryl group. The aryl moiety of the arylalkenyl may be, for example, any aryl group described herein, and the alkenyl moiety of the arylalkenyl may comprise, for example, any of the alkenyl groups described herein. An arylalkenyl group may have 8 to 20 carbon atoms. For example, the alkenyl moiety thereof may have 2 to 6 carbon atoms, and the aryl moiety thereof may have 6 to 14 carbon atoms.

“Arylalkynyl” refers to an acyclic alkynyl group in which one of the hydrogen atoms bonded to a carbon atom, typically a terminal or other sp³ carbon atom, although an sp carbon atom may also be used, is replaced by an aryl group. The aryl moiety of the arylalkynyl may be, for example, any aryl group described herein, and the alkynyl moiety of the arylalkynyl may comprise, for example, any of the alkynyl groups described herein. An arylalkynyl group may have 8 to 20 carbon atoms. For example, the alkynyl moiety thereof may have 2 to 6 carbon atoms, and the aryl moiety thereof may have 6 to 14 carbon atoms.

“Cycloalkyl” refers to a saturated monocycle or polycycle that comprises only carbon atoms in the ring. A cycloalkyl group may have 3 to 7 carbon atoms as a monocycle, 7 to 12 carbon atoms as a bicycle, and up to about 20 carbon atoms as a polycycle. A monocyclic cycloalkyl has 3 to 7 ring atoms, more typically 5 or 6 ring atoms. A bicyclic cycloalkyl may have 7 to 12 ring atoms and may be a fused ring system, a spirocyclic ring system, or a bridged ring system. In exemplary cycloalkyl groups, the atoms may be arranged in a bicyclo[4,5], [5,5], [5,6], or [6,6] system. Non-limiting examples of a monocyclic cycloalkyl include cyclopropyl, cyclobutyl, cyclopentyl, and cyclohexyl (each of which may be substituted or unsubstituted).

The term “substituted” with respect to alkyl, alkylene, aryl, arylalkyl, heterocyclyl, and the like, for example, “substituted alkyl,” “substituted alkylene,” “substituted aryl,” “substituted arylalkyl,” “substituted heterocyclyl,” and “substituted carbocyclyl (e.g., substituted cycloalkyl),” means that at least one hydrogen atom of the alkyl, alkylene, aryl, arylalkyl, heterocyclyl, or carbocyclyl (e.g., cycloalkyl) is each independently replaced by a non-hydrogen substituent. Examples of the typical substituent include halo, haloalkyl, oxo, —CN, —NO₂, ═N—OH, —N₃, —R, —OR, —SR, —N(R)₂, —N(R)₃⁺, ═NR, —NHC(═O)R, —C(═O)R, —C(═O)N(R)₂, —S(═O)₂R, —OS(═O)₂OR, —S(═O)₂OR, —S(═O)₂N(R)₂, —S(═O)R, —OP(═O)(OR)₂, -(alkylene)-C(═O)R, —C(═S)R, —C(═O)OR, -(alkylene)-C(═O)OR, —C(═S)OR, —C(═O)SR, —C(═S)SR, -(alkylene)-C(═O)N(R)₂, —C(═S)N(R)₂, and —C(—NR)N(R)₂, and R is independently H, alkyl, aryl, arylalkyl, or heterocyclyl, but it is not limited thereto. The alkylene, alkenylene, and alkynylene groups may also be similarly substituted.

Those skilled in the art will understand that when a moiety such as “alkyl,” “aryl,” “heterocyclyl,” and the like is substituted with at least one substituent, they may optionally be referred to as a moiety of “alkylene,” “arylene,” “heterocyclylene,” or the like (that is, at least one hydrogen atom of the parent “alkyl,” “aryl,” or “heterocyclyl” moiety is replaced by the substituent as described herein). If the moiety of “alkyl,” “aryl,” “heterocyclyl,” or the like is described herein as “substituted” or depicted in the drawings as substituted (or optionally substituted, for example, the number of substituents is 0 or a positive number), the term “alkyl,” “aryl,” “heterocyclyl,” or the like should be understood to be interchangeable with “alkylene,” “arylene,” “heterocyclylene,” or the like.

Those skilled in the art will recognize that the substituents and other moieties of the compound of Formula I should be selected so as to provide a compound that is sufficiently stable as a pharmaceutically useful compound that can be formulated into an acceptably stable pharmaceutical composition. The compound of Formula I having such stability is to be understood to fall within the scope of the present invention.

“Heteroalkyl” refers to an alkyl group in which at least one carbon atom is replaced by a heteroatom such as O, N, or S. For example, if a carbon atom of the alkyl group attached to a parent molecule is replaced by a heteroatom (e.g., O, N, or S), the resulting heteroalkyl group may be an alkoxy group (e.g., —OCH₃), an amine group (e.g., —NHCH₃, —N(CH₃)₂, or the like), or a thioalkyl group (e.g., —SCH₃), respectively. If a non-terminal carbon atom of the alkyl group that is not attached to a parent molecule is replaced by a heteroatom (e.g., O, N, or S), the resulting heteroalkyl group may be an alkyl ether (e.g., —CH₂CH₂—O—CH₃ or the like), an alkylamine (e.g., —CH₂NHCH₃, —CH₂N(CH₃)₂, or the like), or a thioalkyl ether (e.g., —CH₂—S—CH₃), respectively. If the terminal carbon atom of the alkyl group is replaced by a heteroatom (for example, O, N, or S), the resulting heteroalkyl group may be a hydroxyalkyl group (e.g., —CH₂CH₂—OH), an aminoalkyl group (e.g., —CH₂NH₂), or an alkylthiol group (e.g., —CH₂CH₂—SH), respectively. For example, a heteroalkyl group may have 1 to 20 carbon atoms, 1 to 10 carbon atoms, or 1 to 6 carbon atoms. Preferably, a heteroalkyl group has from 2 to 20, 2 to 10, or 2 to 6 total atoms in the chain (i.e., carbon atoms plus heteroatoms combined). A C₁-C₆ heteroalkyl group refers to a heteroalkyl group having 1 to 6 carbon atoms.

The term “heterocycle” or “heterocyclyl” used herein includes those described in the documents such as Paquette, Leo A., Principles of Modern Heterocyclic Chemistry (W. A. Benjamin, New York, 1968), specifically Chapters 1, 3, 4, 6, 7, and 9; The Chemistry of Heterocyclic Compounds, A Series of Monographs (John Wiley & Sons, New York, from 1950 to the present), specifically Volumes 13, 14, 16, 19, and 28; and J. Am. Chem. Soc. (1960) 82:5566, but it is not limited thereto. In a specific embodiment of the present invention, “heterocycle” includes carbocycle as defined herein in which at least one (e.g., 1, 2, 3, or 4) carbon atom is replaced by a heteroatom (e.g., O, N, or S). The term “heterocycle” or “heterocyclyl” includes saturated, partially unsaturated, and aromatic rings (i.e., a heteroaromatic ring). Substituted heterocycle, for example, includes a heterocyclic ring substituted with any of the substituents disclosed herein, inclusive of a carbonyl group.

Exemplary heterocycles include pyridyl, dihydropyridyl, tetrahydropyridyl(piperidyl), thiazolyl, tetrahydrothiophenyl, sulfur-oxidized tetrahydrothiophenyl, pyrimidinyl, furanyl, thienyl, pyrrolyl, pyrazolyl, imidazolyl, tetrazolyl, benzofuranyl, thianaphthenyl, indolyl, indolenyl, quinolinyl, isoquinolinyl, benzimidazolyl, piperidinyl, 4-piperidinyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocynyl, triazinyl, 6H-1,2,5-thiadiazinyl, 2H,6H-1,5,2-dithiazinyl, thianthrenyl, pyranyl, isobenzofuranyl, chromenyl, xanthenyl, phenoxatinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, phtheridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, and benzoxazolinyl (each of which may be substituted or unsubstituted), but it is not limited thereto.

As an example, a carbon-bonded heterocycle may be bonded at the 2, 3, 4, 5, or 6-position of pyrazine, at the 3, 4, 5, or 6-position of pyridazine, at the 2, 4, 5, or 6-position of pyrimidine, at the 2, 3, 5, or 6-position of pyrazine, at the 2, 3, 4, or 5-position of furan, tetrahydrofuran, thiofuran, thiophene, pyrrole, or tetrahydropyrrole, at the 2, 4, or 5-position of oxazole, imidazole, or thiazole, at the 3, 4, or 5-position of isoxazole, pyrazole, or isothiazole, at the 2 or 3-position of aziridine, at the 2, 3, or 4-position of azetidine, at the 2, 3, 4, 5, 6, 7, or 8-position of quinoline, or at the 1, 3, 4, 5, 6, 7, or 8-position of isoquinoline, but it is not limited thereto. More typically, examples of a carbon-bonded heterocycle include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, and 5-thiazolyl (each of which may be substituted or unsubstituted).

As an example, a nitrogen-bonded heterocycle may be bonded at the 1-position of aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole, at the 2-position of isoindole or isoindoline, at the 4-position of morpholine, and at the 9-position of carbazole or β-carboline (each of which may be substituted or unsubstituted), but it is not limited thereto. More typically, examples of a nitrogen-bonded heterocycle include 1-aziridinyl, 1-azetidyl, 1-pyrrolyl, 1-imidazolyl, 1-pyrazolyl, and 1-piperidinyl (each of which may be substituted or unsubstituted).

“Heterocyclylalkyl” refers to an acyclic alkyl radical in which one hydrogen atom bonded to a carbon atom, typically a terminal or sp³ carbon atom, is replaced by a heterocyclyl radical (i.e., a heterocyclyl-alkylene moiety). Examples of a typical heterocyclylalkyl group include heterocyclyl-CH₂—, 2-(heterocyclyl)ethan-1-yl, and the like, but it is not limited thereto. The “heterocyclyl” moiety thereof used herein includes those described in the document such as “Principles of Modern Heterocyclic Chemistry” and any heterocyclyl group described above. Those skilled in the art will understand that if the resulting group is chemically stable, the heterocyclyl group may be attached to the alkyl moiety of the heterocyclylalkyl through a carbon-to-carbon bond or a carbon-to-heteroatom bond. A heterocyclylalkyl group may have 2 to 20 carbon atoms. For example, the alkyl moiety of the heterocyclylalkyl group may have 1 to 6 carbon atoms, and the heterocyclyl moiety thereof may have 2 to 14 carbon atoms. Examples of the heterocyclylalkyl include a 5-membered heterocycle containing sulfur, oxygen, and/or nitrogen such as thiazolylmethyl, 2-thiazolylethan-1-yl, imidazolylmethyl, oxazolylmethyl, thiadiazolylmethyl, and the like; and a 6-membered heterocycle containing sulfur, oxygen, and/or nitrogen such as piperidinylmethyl, piperazinylmethyl, morpholinylmethyl, pyridinylmethyl, pyridazylmethyl, pyrimidylmethyl, pyrazinylmethyl, and the like (each of which may be substituted or unsubstituted), but it is not limited thereto.

“Heterocyclylalkenyl” refers to an acyclic alkenyl radical in which one of the hydrogen atoms bonded to a carbon atom, typically a terminal or sp³ carbon atom although an sp² carbon atom may also be used, is replaced by a heterocyclyl radical (i.e., a heterocyclyl-alkenylene moiety). The heterocyclyl moiety of the heterocyclylalkenyl group includes those described in the document such as “Principles of Modern Heterocyclic Chemistry” and any heterocyclyl group described herein. The alkenyl moiety of the heterocyclylalkenyl group includes any alkenyl group described herein. Those skilled in the art will understand that if the resulting group is chemically stable, the heterocyclyl group may be attached to the alkenyl moiety of the heterocyclylalkenyl via a carbon-to-carbon bond or a carbon-to-heteroatom bond. A heterocyclylalkenyl group may have 3 to 20 carbon atoms. For example, the alkenyl moiety of the heterocyclylalkenyl group may have 2 to 6 carbon atoms, and the heterocyclyl moiety thereof may have 2 to 14 carbon atoms.

“Heterocyclylalkynyl” refers to an acyclic alkynyl radical in which one of the hydrogen atoms bonded to a carbon atom, typically a terminal or sp³ carbon atom although an sp carbon atom may also be used, is replaced by a heterocyclyl radical (i.e., a heterocyclyl-alkynylene moiety). The heterocyclyl moiety of the heterocyclylalkynyl group includes those described in the document such as “Principles of Modern Heterocyclic Chemistry” and any heterocyclyl group described herein. The alkynyl moiety of the heterocyclylalkynyl group includes any alkynyl group described herein. Those skilled in the art will understand that if the resulting group is chemically stable, the heterocyclyl group may be attached to the alkynyl moiety of the heterocyclylalkynyl via a carbon-to-carbon bond or a carbon-to-heteroatom bond. A heterocyclylalkynyl group may have 3 to 20 carbon atoms. For example, the alkynyl moiety of the heterocyclylalkynyl group may have 2 to 6 carbon atoms, and the heterocyclyl moiety thereof may have 2 to 14 carbon atoms.

“Heteroaryl” refers to an aromatic heterocyclyl containing at least one heteroatom in the ring. Non-limiting examples of a suitable heteroatom that may be contained in the aromatic ring include oxygen, sulfur, and nitrogen. Non-limiting examples of a heteroaryl ring include all of those enumerated in the definition of “heterocyclyl” herein, inclusive of pyridinyl, pyrrolyl, oxazolyl, indolyl, isoindolyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, and the like (each of which may be substituted or unsubstituted).

“Carbocycle” or “carbocyclyl” refers to a saturated, partially unsaturated, or aromatic ring having 3 to 7 carbon atoms as a monocycle, 7 to 12 carbon atoms as a bicycle, and up to about 20 carbon atoms as a polycycle. A monocyclic carbocycle has 3 to 7 ring atoms, more typically 5 or 6 ring atoms. A bicyclic cycloalkyl may have 7 to 12 ring atoms and may be a fused ring system, a spirocyclic ring system, or a bridged ring system. In exemplary cycloalkyl groups, the atoms are arranged in a bicyclo[4,5], [5,5], [5,6], or [6,6] system. Non-limiting examples of a monocyclic cycloalkyl include cyclopropyl, cyclobutyl, cyclopentyl, and cyclohexyl (each of which may be substituted or unsubstituted).

“Acyl” refers to —C(═O)-alkyl, —C(═O)-carbocycle (which is substituted or unsubstituted), and —C(═O)-heterocycle (which is substituted or unsubstituted), wherein the alkyl, carbocycle, or heterocycle moiety is as defined herein. Non-limiting examples of “acyl” include —C(═O)CH₃, —C(═O)CH₂CH₃, —C(═O)CH(CH₃)₂, —C(═O)C(CH₃)₃, —C(═O)-phenyl (which is substituted or unsubstituted), —C(═O)-cyclopropyl (which is substituted or unsubstituted), —C(═O)-cyclobutyl (which is substituted or unsubstituted), —C(═O)-cyclopentyl (which is substituted or unsubstituted), —C(═O)-cyclohexyl (which is substituted or unsubstituted), and —C(═O)-pyridyl (which is substituted or unsubstituted).

“Arylheteroalkyl” refers to a heteroalkyl as defined herein, wherein a hydrogen atom (which may be attached to either a carbon atom or a heteroatom) is replaced by an aryl group as defined herein. If the resulting group is chemically stable, the aryl group may be attached to a carbon atom of the heteroalkyl group or the heteroatom of the heteroalkyl group. For example, an arylheteroalkyl group may have a formula of -alkylene-O-aryl, -alkylene-O-alkylene-aryl, -alkylene-NH-aryl, -alkylene-NH-alkylene-aryl, -alkylene-S-aryl, -alkylene-S-alkylene-aryl, or the like. In addition, any alkylene moiety in the above formulae may be further substituted with any of the substituents defined or exemplified herein.

“Heteroarylalkyl” refers to an alkyl group as defined herein, wherein a hydrogen atom is replaced by a heteroaryl group as defined herein. Non-limiting examples of heteroarylalkyl include —CH₂-pyridinyl, —CH₂-pyrrolyl, —CH₂-oxazolyl, —CH₂-indolyl, —CH₂-isoindolyl, —CH₂-furanyl, —CH₂-thienyl, —CH₂-benzofuranyl, —CH₂-benzothiophenyl, —CH₂-carbazolyl, —CH₂-imidazolyl, —CH₂-thiazolyl, —CH₂-isoxazolyl, —CH₂-pyrazolyl, —CH₂-isothiazolyl, —CH₂-quinolyl, —CH₂-isoquinolyl, —CH₂-pyridazyl, —CH₂-pyrimidyl, —CH₂-pyrazyl, —CH(CH₃)-pyridinyl, —CH(CH₃)-pyrrolyl, —CH(CH₃)-oxazolyl, —CH(CH₃)-indolyl, —CH(CH₃)-isoindolyl, —CH(CH₃)-furanyl, —CH(CH₃)-thienyl, —CH(CH₃)-benzofuranyl, —CH(CH₃)-benzothiophenyl, —CH(CH₃)-carbazolyl, —CH(CH₃)-imidazolyl, —CH(CH₃)-thiazolyl, —CH(CH₃)-isoxazolyl, —CH(CH₃)-pyrazolyl, —CH(CH₃)-isothiazolyl, —CH(CH₃)-quinolyl, —CH(CH₃)-isoquinolyl, —CH(CH₃)-pyridazyl, —CH(CH₃)-pyrimidyl, —CH(CH₃)-pyrazyl, and the like.

“Silyloxy” refers to the group —O—SiR₃, wherein each R independently is alkyl, aryl (which is substituted or unsubstituted), or heteroaryl (which is substituted or unsubstituted). Non-limiting examples of silyloxy include —O—Si(CH₃)₃, —O—Si(CH₃)₂tBu, —O—Si(tBu)₂CH₃, —O—Si(tBu)₃, —O—Si(CH₃)₂Ph, —O—Si(Ph)₂CH₃, and —O—Si(Ph)₃.

The term “optionally substituted” refers to a particular moiety (e.g., an optionally substituted aryl group) of the compound of Formula I that optionally has one, two, or more substituents.

The term “ester thereof” refers to any ester of a compound wherein any —COOH functional group of the molecule is modified to be a —COOR functional group or any —OH functional group of the molecule is modified to be a —OC(═O)R. Here, the R moiety of the ester may be any carbon-containing group that forms a stable ester moiety, which includes, but is not limited to, alkyl, alkenyl, alkynyl, cycloalkyl, cycloalkylalkyl, aryl, arylalkyl, heterocyclyl, heterocyclylalkyl, and substituted derivatives thereof. Examples of the ester may also include an ester such as those described above of a “tautomeric enol” as described below.

### Compounds

In certain embodiments, the invention provides a compound represented by Formula (I):

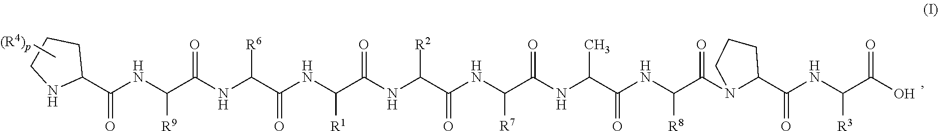

or a pharmaceutically acceptable salt thereof
wherein:

R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl;

R⁴, independently for each occurrence, is selected from substituted or unsubstituted alkyl, aryl, arylalkyl, heterocyclyl, oxo, —OR^{b}, —CH₂OR^{b}, halo, hydroxyl, and hydroxyalkyl;
R^{b} is substituted or unsubstituted alkyl, aryl, arylalkyl, or heterocyclyl;

p is 0, 1, or 2;

R⁶ is hydrogen or substituted or unsubstituted alkyl; and

R⁷, R⁸, and R⁹ are each independently hydrogen or alkyl.

preferably wherein the compound comprises at least one D-amino acid residue.

In certain embodiments, the invention provides a compound represented by Formula (I), or a pharmaceutically acceptable salt thereof, wherein the compound is not:

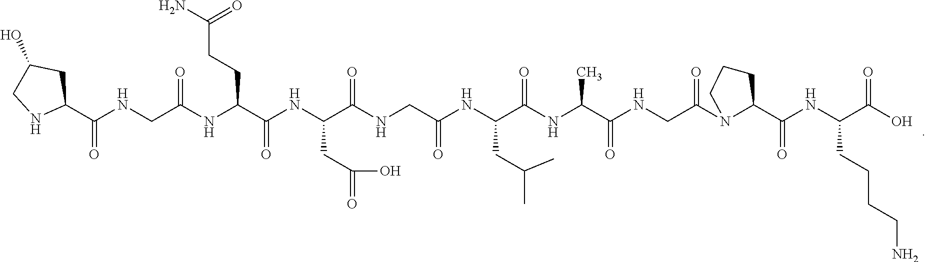 [SMILES: CC(C)C[C@H](NC(=O)CNC(=O)[C@H](CC(=O)O)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O]

In certain embodiments, R¹, R², and R³ are each independently H or substituted or unsubstituted alkyl, arylalkyl, or heterocyclylalkyl;

R⁴, independently for each occurrence, is selected from substituted or unsubstituted alkyl, oxo, hydroxyl, —OR^{b}, hydroxyalkyl, —CH₂OR^{b}, and halo;
R^{b} is substituted or unsubstituted alkyl, aryl, arylalkyl, or heterocyclyl;

R⁶ is hydrogen or substituted or unsubstituted alkyl; and

R⁷, R⁸, and R⁹ are each independently hydrogen or alkyl.

In some embodiments, where indicated, alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl is unsubstituted or is substituted with one or more substituents selected from halo, haloalkyl, oxo, —CN, —NO₂, ═N—OH, —N₃, —R^{a}, —OR^{a}, —SR^{a}, —N(R^{a})₂, —N(R^{a})₃⁺, —NR^{a}, —NHC(═O)R^{c}, —C(═O)R^{c}, —C(═O)N(R^{a})₂, —S(═O)₂R^{c}, —OS(═O)₂OR^{a}, —S(═O)₂OR^{a}, —S(═O)₂N(R^{a})₂, —S(═O)R^{c}, —OP(═O)(OR^{a})₂, -(alkylene)-C(═O)R^{c}, —C(═S)R^{c}, —C(═O)OR^{a}, -(alkylene)-C(═O)OR^{a}, —C(═S)OR^{a}, —C(═O)SR^{a}, —C(═S)SR^{a}, -(alkylene)-C(═O)N(R^{a})₂, —C(═S)N(R^{a})₂, and —C(—NR^{a})N(R^{a})₂;

R^{a}, independently for each occurrence, is hydrogen, or substituted or unsubstituted alkyl, aryl, arylalkyl, heterocyclyl, heterocyclylalkyl, cycloalkyl, or (cycloalkyl)alkyl; and

R^{c}, independently for each occurrence, is substituted or unsubstituted alkyl, aryl, arylalkyl, heterocyclyl, heterocyclylalkyl, cycloalkyl, or (cycloalkyl)alkyl.

In more particular embodiments, where indicated, alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl is unsubstituted or is substituted with one or more substituents selected from halo, haloalkyl, oxo, —R^{a}, —OR^{a}, —N(R^{a})₂, —N(R^{a})₃⁺, —NHC(═O)R^{c}, —C(═O)R^{c}, —C(═O)N(R^{a})₂, —C(═O)OR^{a}, -(alkylene)-C(═O)OR^{a}, and -(alkylene)-C(═O)N(R^{a})₂;

R^{a}, independently for each occurrence, is hydrogen, or substituted or unsubstituted alkyl, aryl, arylalkyl, heterocyclyl, heterocyclylalkyl, cycloalkyl, or (cycloalkyl)alkyl; and

R^{c}, independently for each occurrence, is substituted or unsubstituted alkyl, aryl, arylalkyl, heterocyclyl, heterocyclylalkyl, cycloalkyl, or (cycloalkyl)alkyl.

In certain such embodiments, R^{a}, independently for each occurrence, is hydrogen, alkyl, aryl, arylalkyl, heterocyclyl, or heterocyclylalkyl; and R^{c}, independently for each occurrence, is alkyl, aryl, arylalkyl, heterocyclyl, or heterocyclylalkyl.

In certain embodiments, the compound has the structure of formula (I-10L):

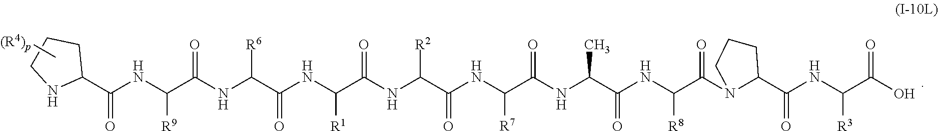

Alternatively, the compound may have the structure of formula (I-10D):

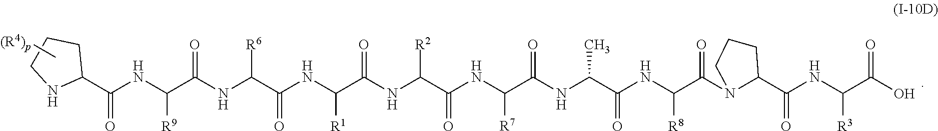

In certain embodiments, R¹ is substituted or unsubstituted alkyl, arylalkyl, or heterocyclylalkyl.

More specifically, R¹ may be selected from substituted or unsubstituted alkyl,

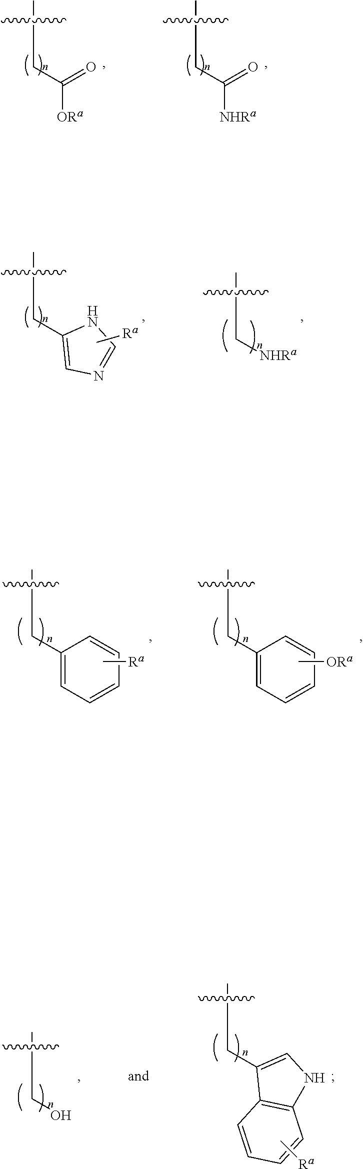

R^{a} is hydrogen or alkyl; and
n is an integer from 1 to 10, preferably 1-5, more preferably 1-3.

Exemplary R¹ groups include

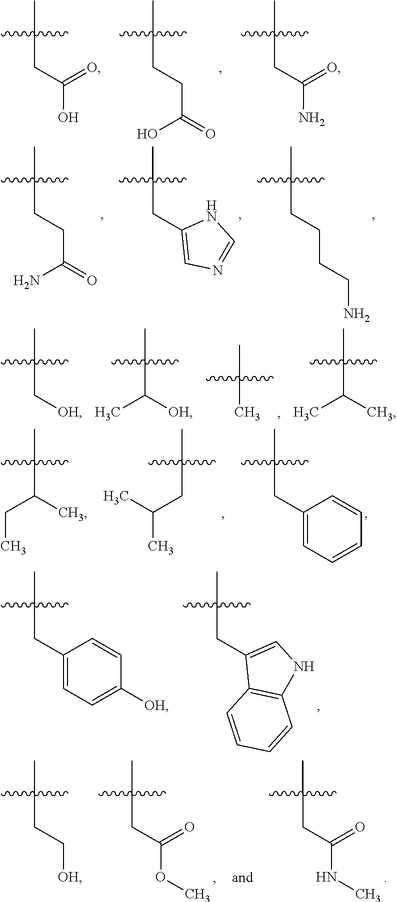

In some preferred embodiments, R¹ is

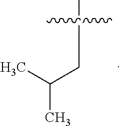

In alternative preferred embodiments, R¹ is

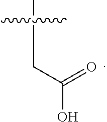

In certain embodiments, the compound has the structure of formula (I-1L).

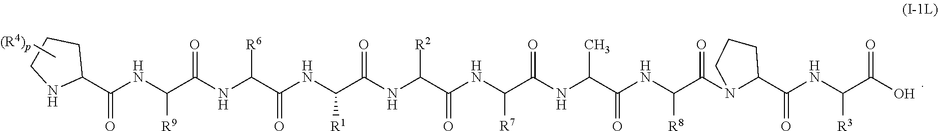

Alternatively, the compound may have the structure of formula (I-1D)

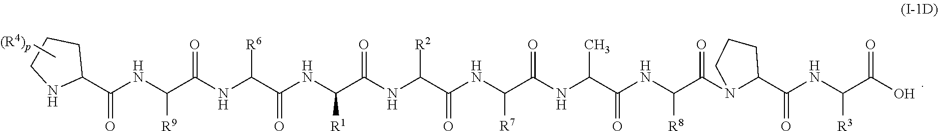

In certain embodiments, R² is H or substituted or unsubstituted alkyl, arylalkyl, or heterocyclylalkyl.

In some embodiments, R² is selected from hydrogen, substituted or unsubstituted alkyl,

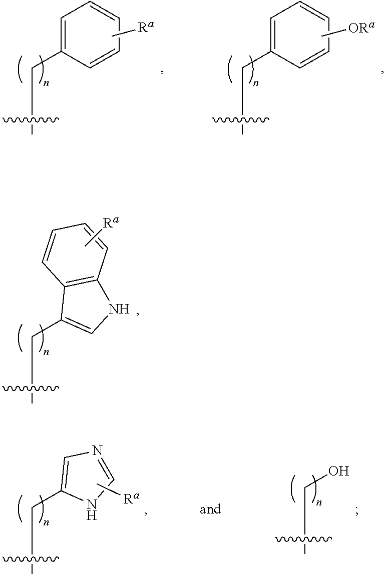

R^{a} is hydrogen or alkyl; and
n is an integer from 1 to 10, preferably 1-5, more preferably 1-3.

Exemplary R² groups include

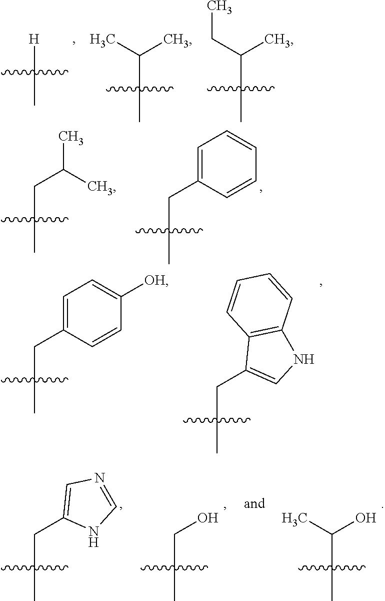

Preferably, R² is hydrogen.

In certain embodiments, the compound has the structure of formula (I-2L):

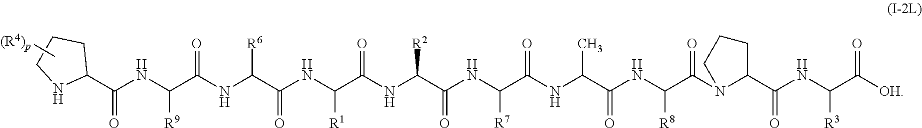

Alternatively, the compound may have the structure of formula (I-2D):

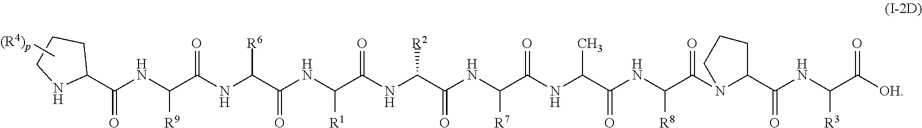

In certain embodiments, R³ is substituted or unsubstituted alkyl or arylalkyl.

In some embodiments, R³ is selected from substituted or unsubstituted alkyl,

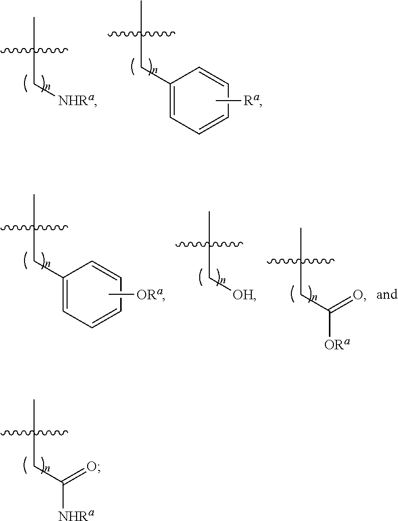

R^{a} is hydrogen or alkyl; and
n is an integer from 1 to 10, preferably 1-5, more preferably 1-3.

Exemplary R³ groups include

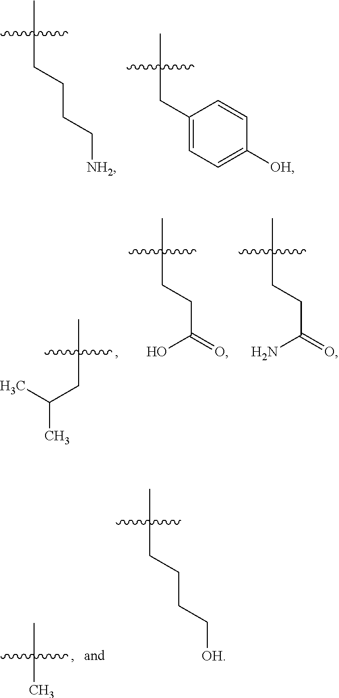

### Preferably, R³ is

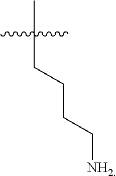 [SMILES: *CCCCN]

In certain embodiments, the compound has the structure of formula (I-3L):

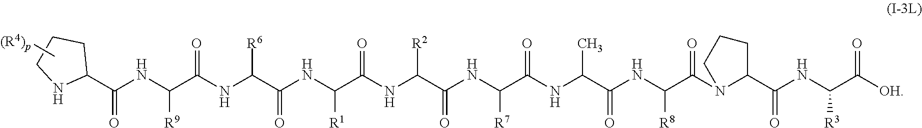

Alternatively, the compound may have the structure of formula (I-3D):

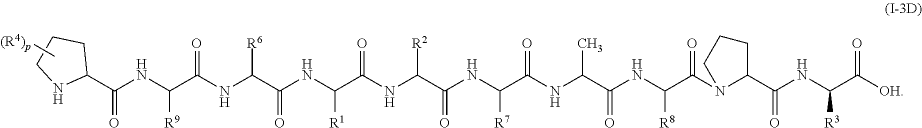

In certain embodiments, p is 1 or 2; and R⁴, independently for each occurrence, is selected from substituted or unsubstituted alkyl, —OR^{b}, —CH₂OR^{b}, halo, hydroxyl, and hydroxyalkyl.

In certain embodiments, p is 1 or 2; and R⁴, independently for each occurrence, is selected from —CH₃, halo, hydroxyl, and hydroxyalkyl.

In certain preferred embodiments, R⁴ is hydroxyl. In alternative preferred embodiments, R⁴ is —CH₃.

In any of the above embodiments, p may be 1.

In certain embodiments, the compound has the structure of formula (I-4Lg):

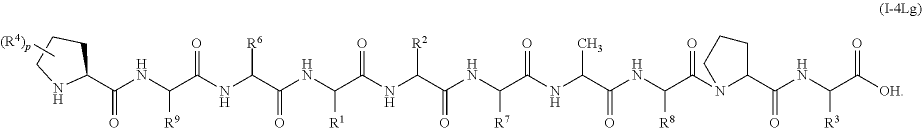

In certain embodiments, the compound has the structure of formula (I-4La):

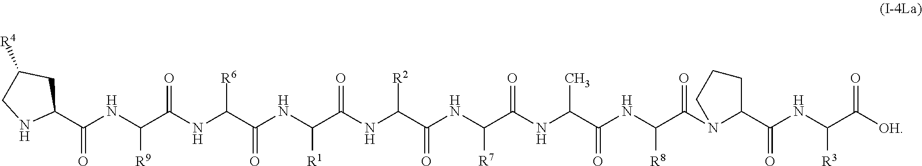

In certain embodiments, the compound has the structure of formula (I-4Lb):

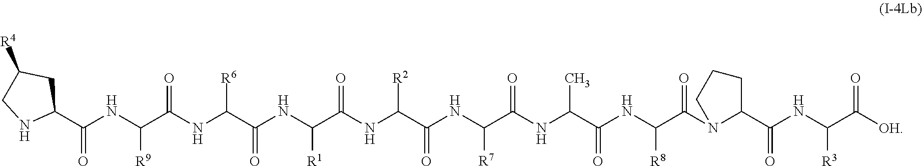

In certain embodiments, the compound has the structure of formula (I-4Lc):

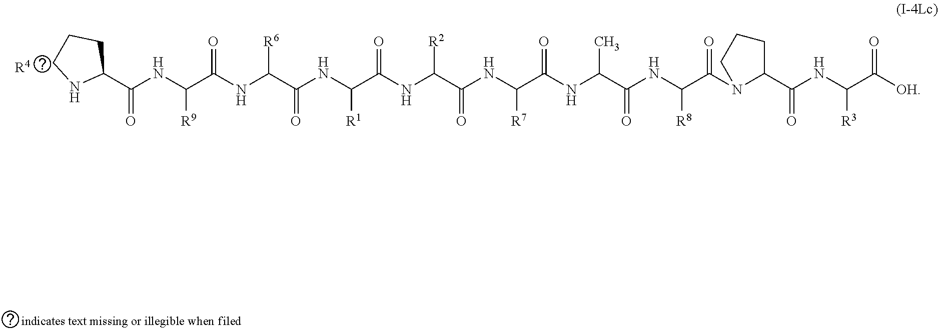

In certain embodiments, the compound has the structure of formula (I-4Lc), provided that R⁴ is not hydroxyl.

In certain embodiments, the compound has the structure of formula (I-4Dg):

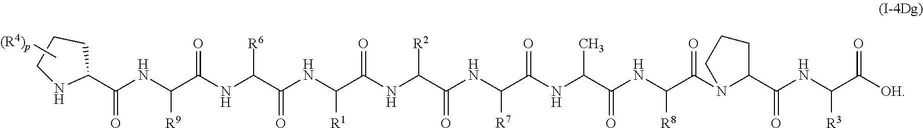

In certain embodiments, the compound has the structure of formula (I-4Da):

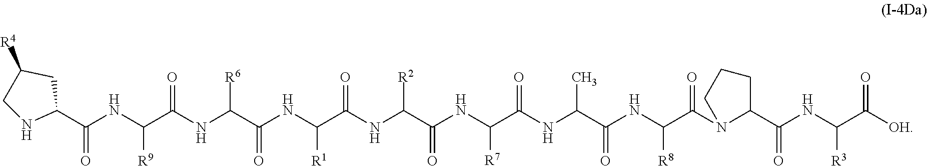

In certain embodiments, the compound has the structure of formula (I-4Db):

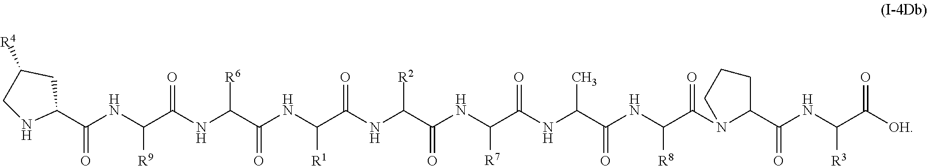

In certain embodiments, the compound has the structure of formula (I-4Dc):

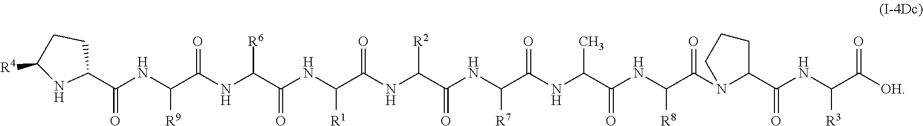

In certain embodiments, the compound has the structure of formula (I-4Dc), provided that R⁴ is not hydroxyl.

In certain embodiments, R⁴ is oxo.

In certain embodiments, the compound has the structure of formula (I-4Ld):

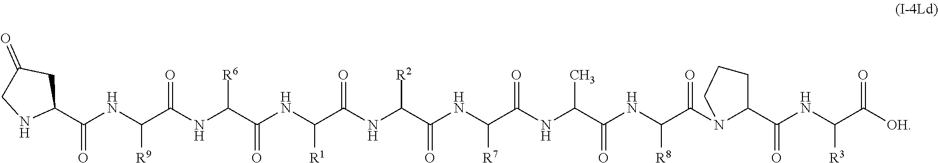

In certain embodiments, the compound has the structure of formula (I-4Le):

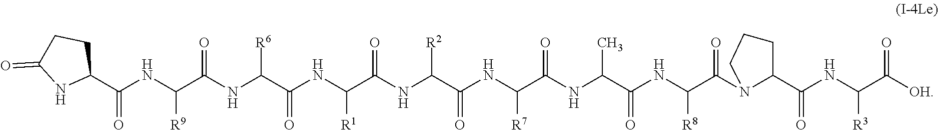

In certain embodiments, the compound has the structure of formula (I-4Dd):

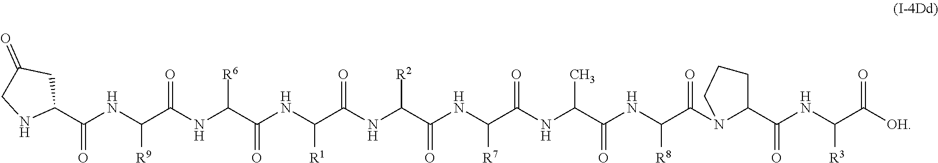

In certain embodiments, the compound has the structure of formula (I-4De):

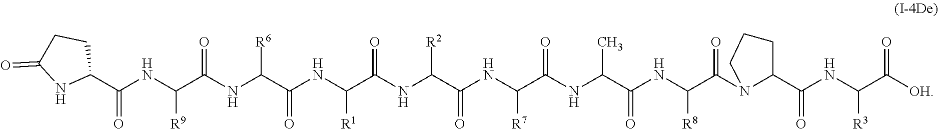

In certain embodiments, R⁶ is hydrogen or alkyl, wherein the alkyl is optionally substituted with one occurrence of —C(═O)NH₂. In certain embodiments, wherein R⁶ is alkyl optionally substituted with one occurrence of —C(═O)NH₂. For example, R⁶ may be —CH₃. Alternatively, R⁶ may be

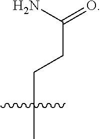 [SMILES: *CCC(N)=O]

In certain embodiments, the compound has the structure of formula (I-6L):

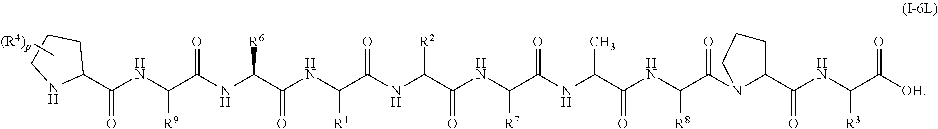

Alternatively, the compound may have the structure of formula (I-6D):

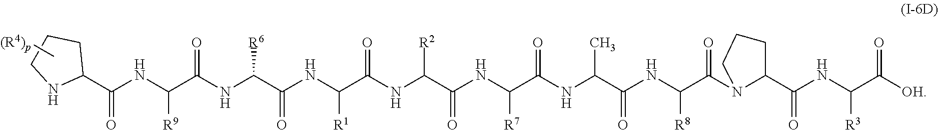

In certain embodiments, R⁷ is (C₁-C₁₀)alkyl, preferably

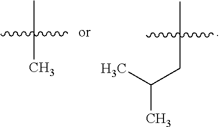

In certain embodiments, the compound has the structure of formula (I-7L):

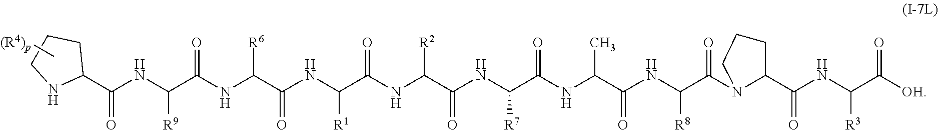

Alternatively, the compound may have the structure of formula (I-7D):

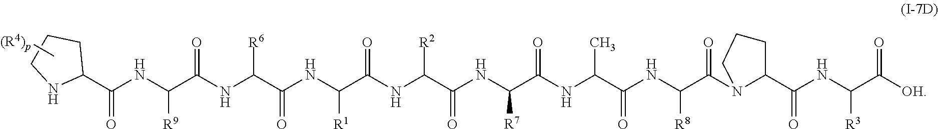

In certain embodiments, the compound has the structure of formula (I-11L):

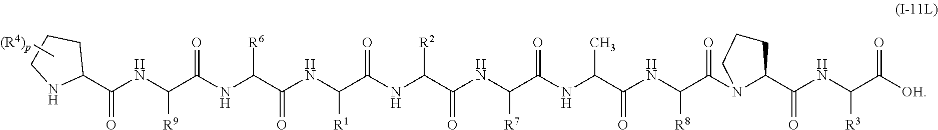

Alternatively, the compound may have the structure of formula (I-11D):

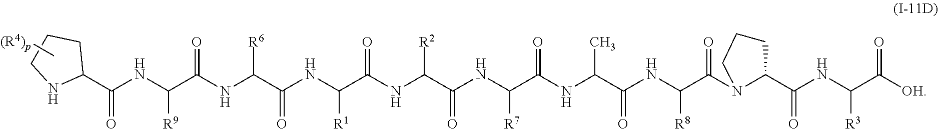

In certain embodiments, R⁸ is —CH₃ or —H, preferably —H.

In certain embodiments, R⁹ is —CH₃ or —H, preferably —H.

In certain embodiments, the compound comprises at least one, at least two, at least three, at least four, at least five, at least six, at least seven, or at least eight D-amino acid residues.

In certain embodiments, the invention provides a compound of formula (I), or a pharmaceutically acceptable salt thereof, wherein the compound is selected from the following:

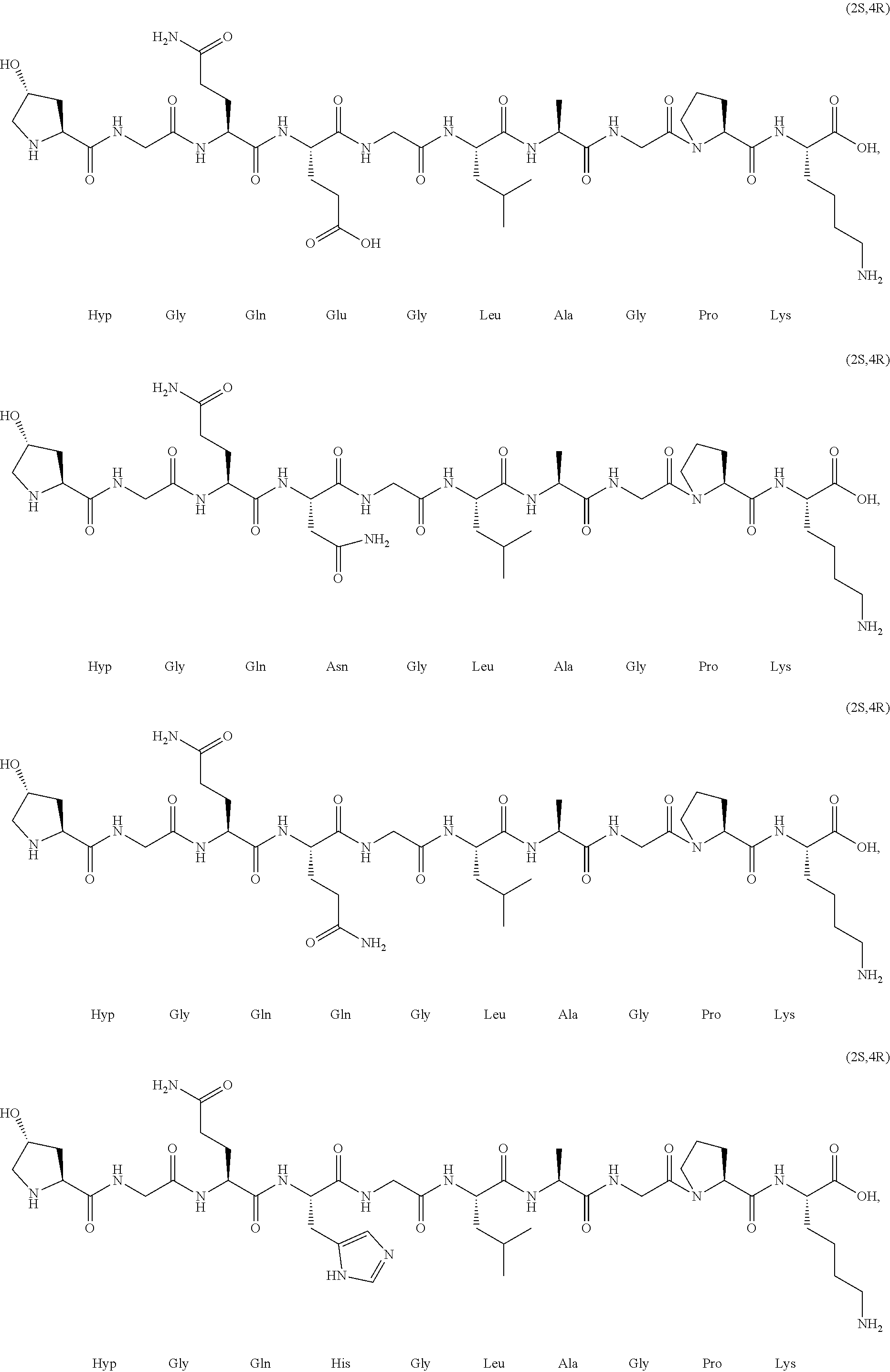

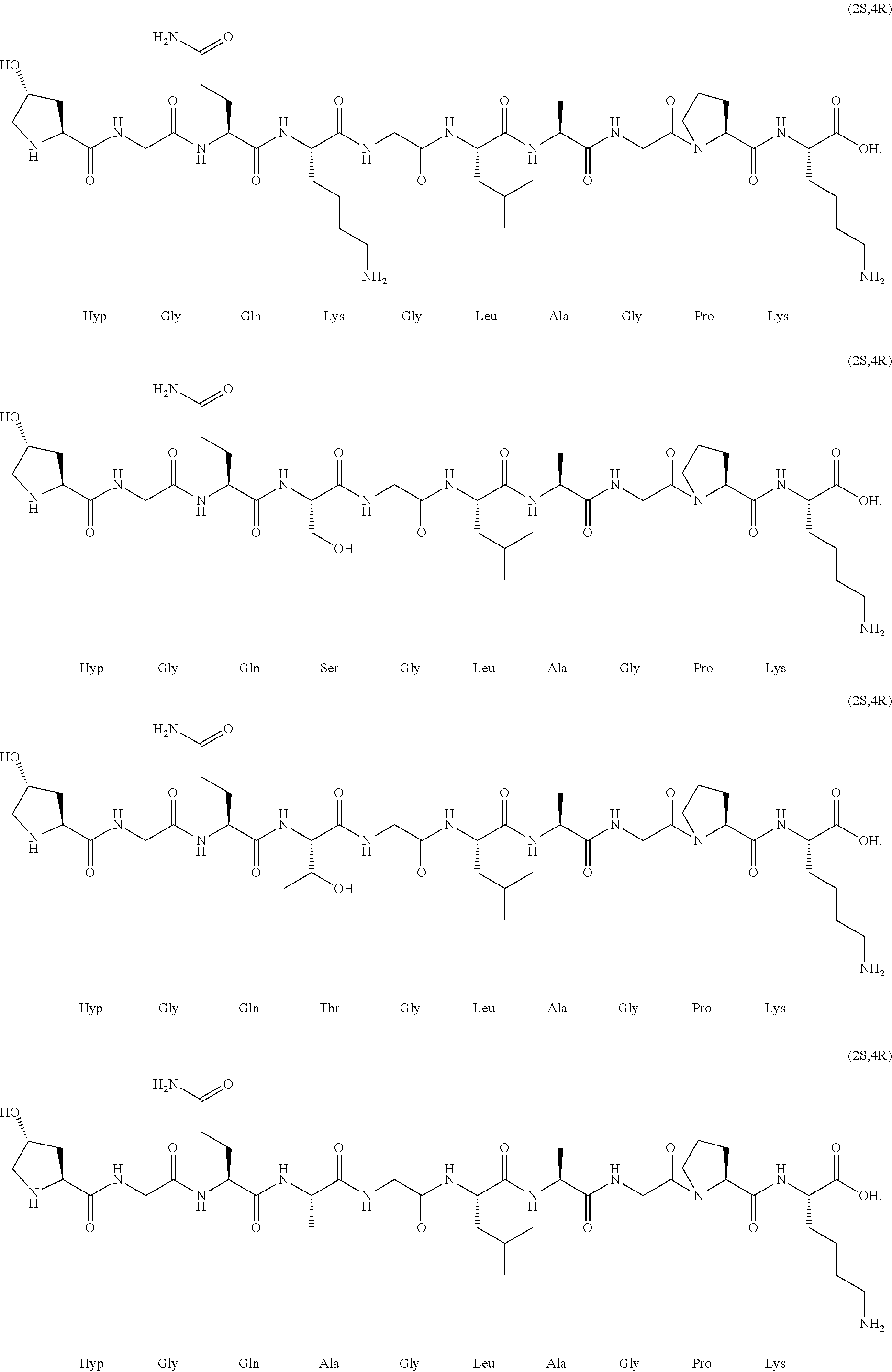

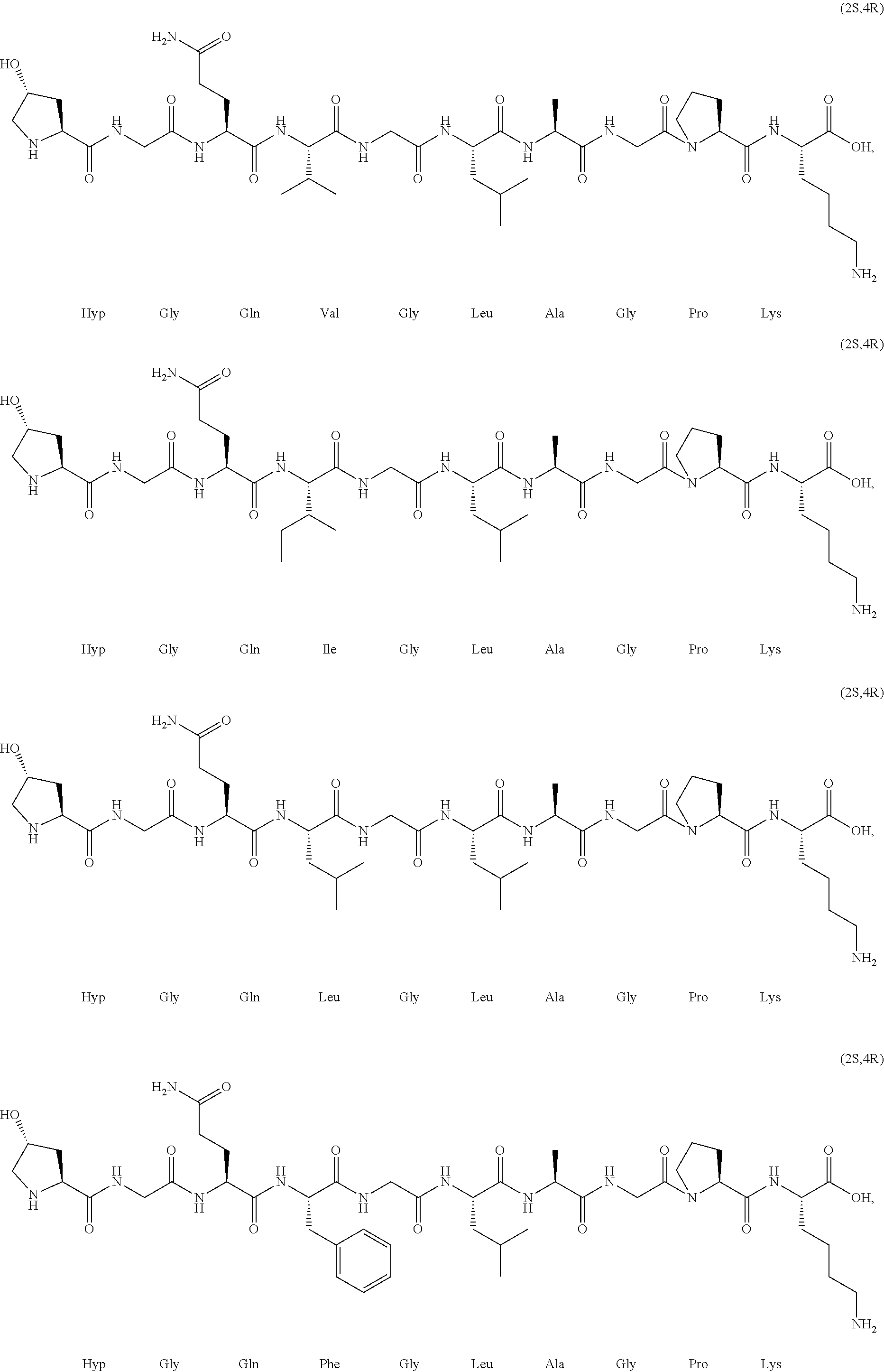

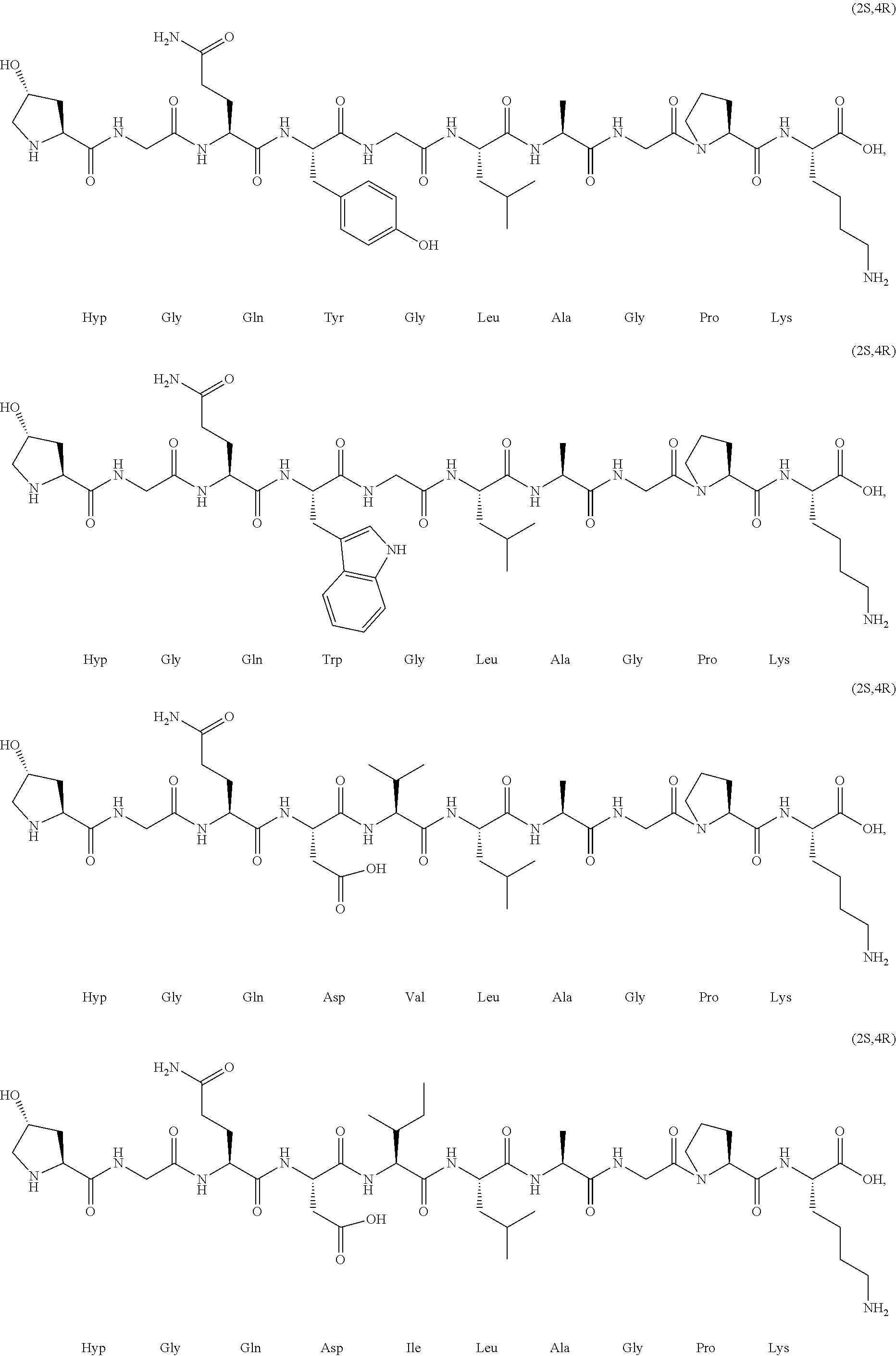

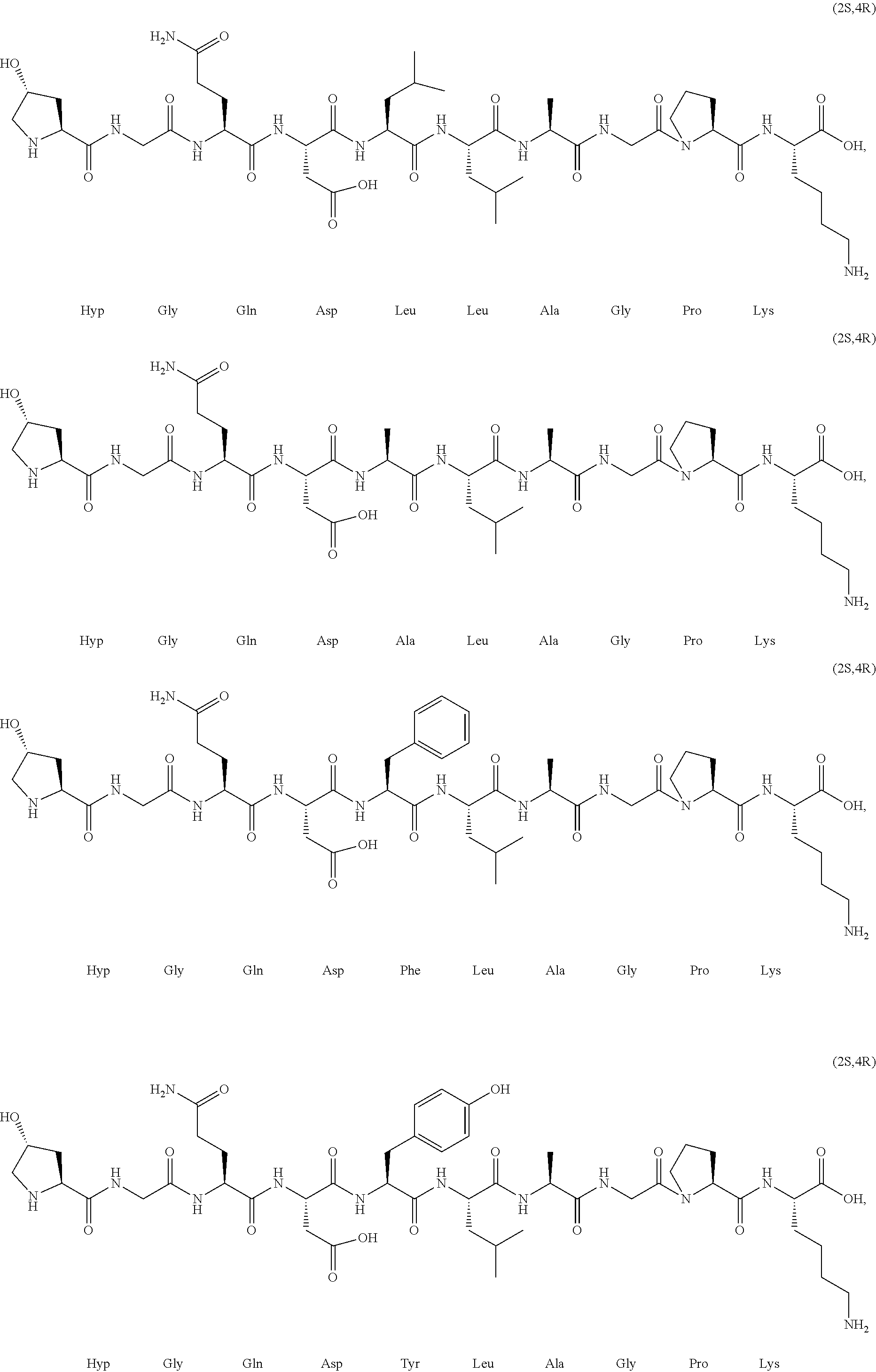

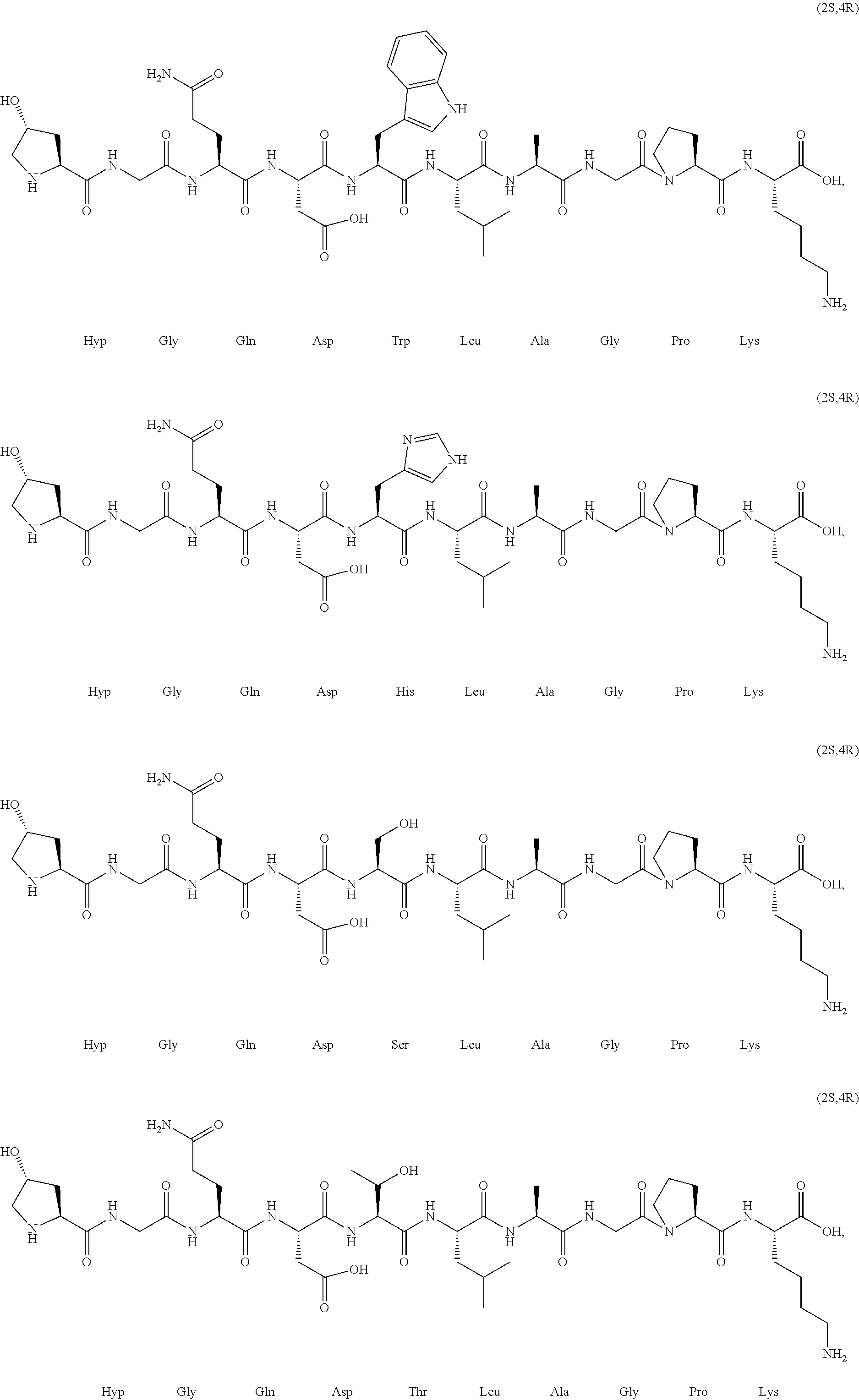

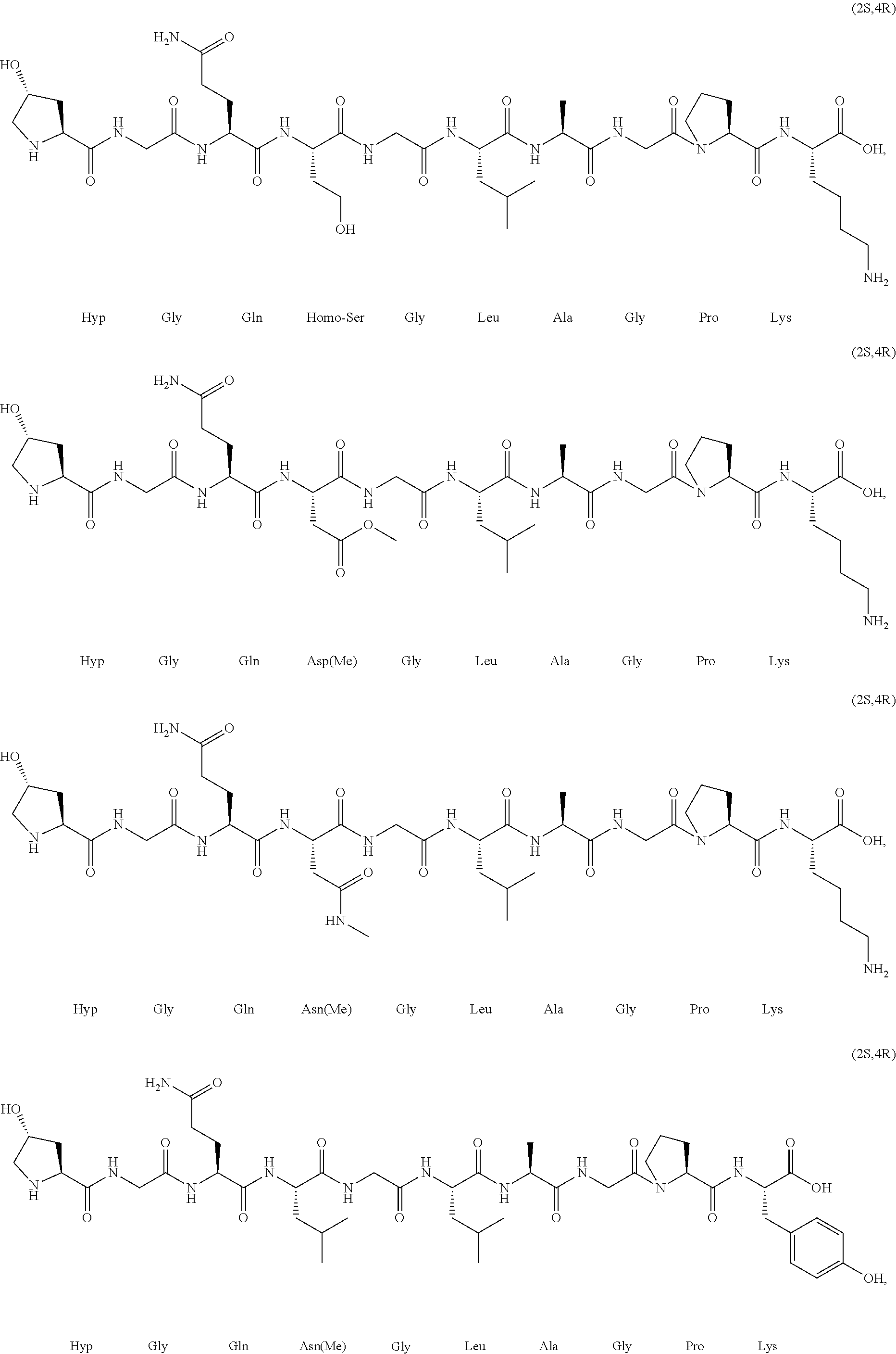

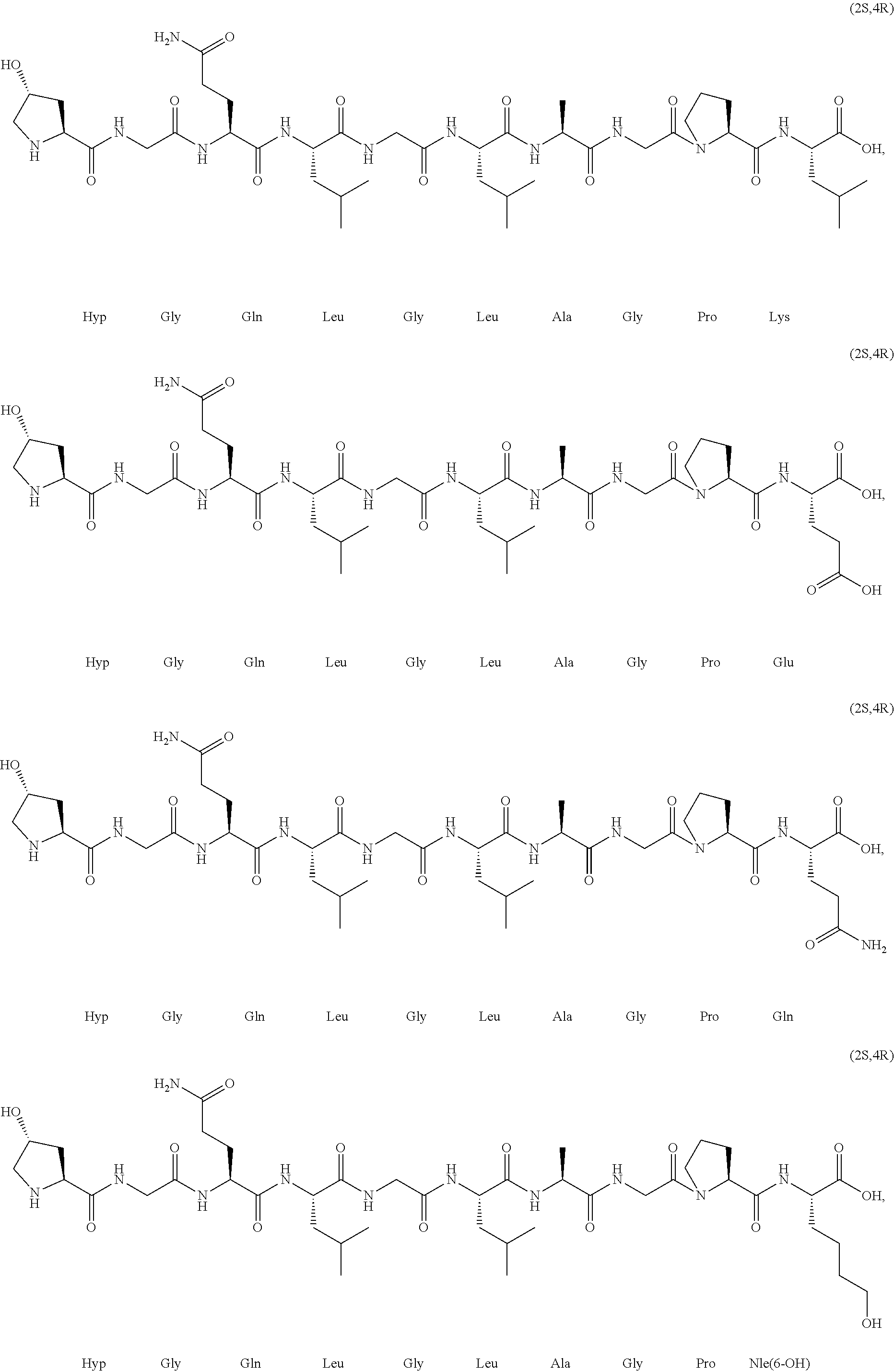

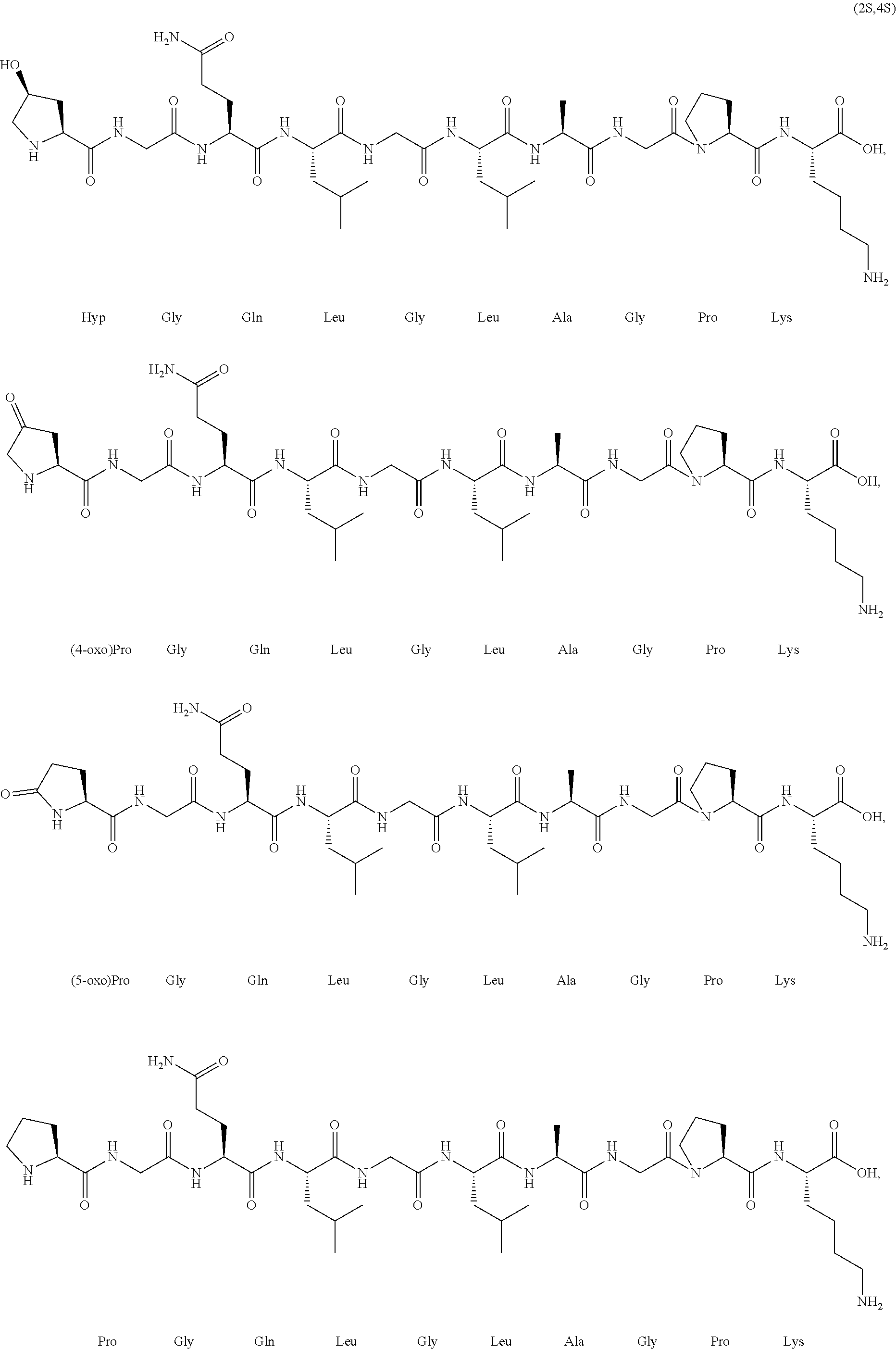

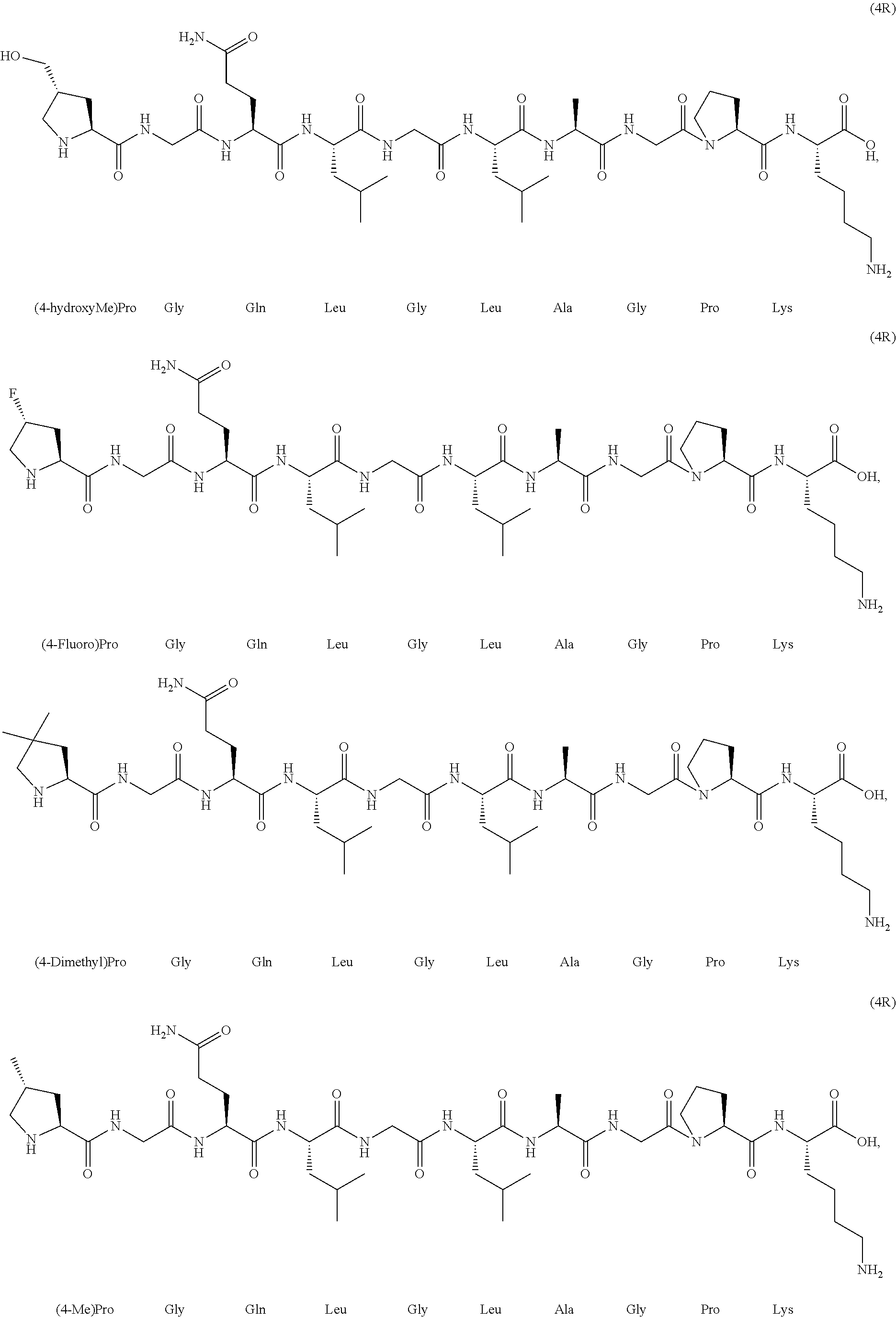

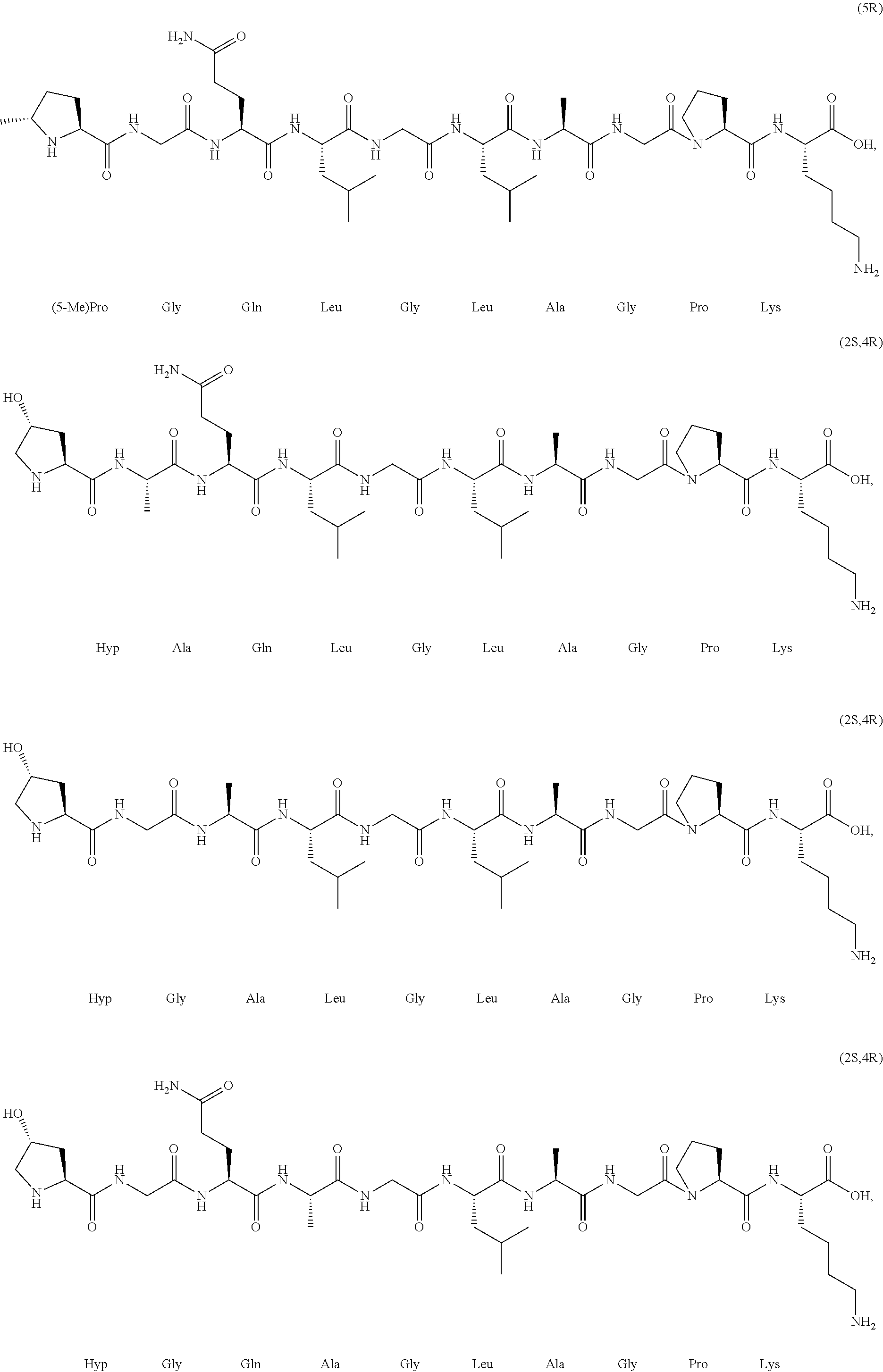

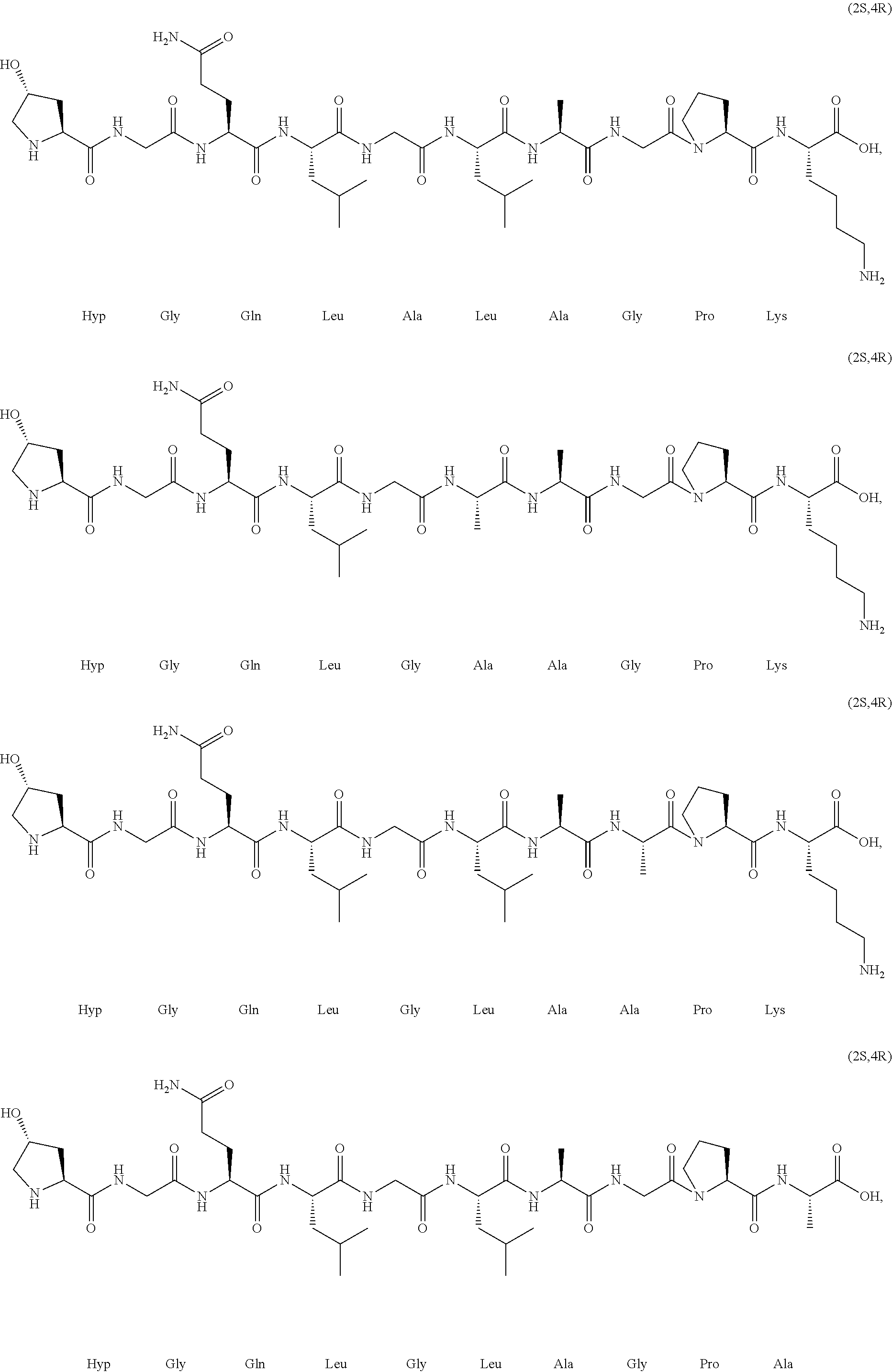

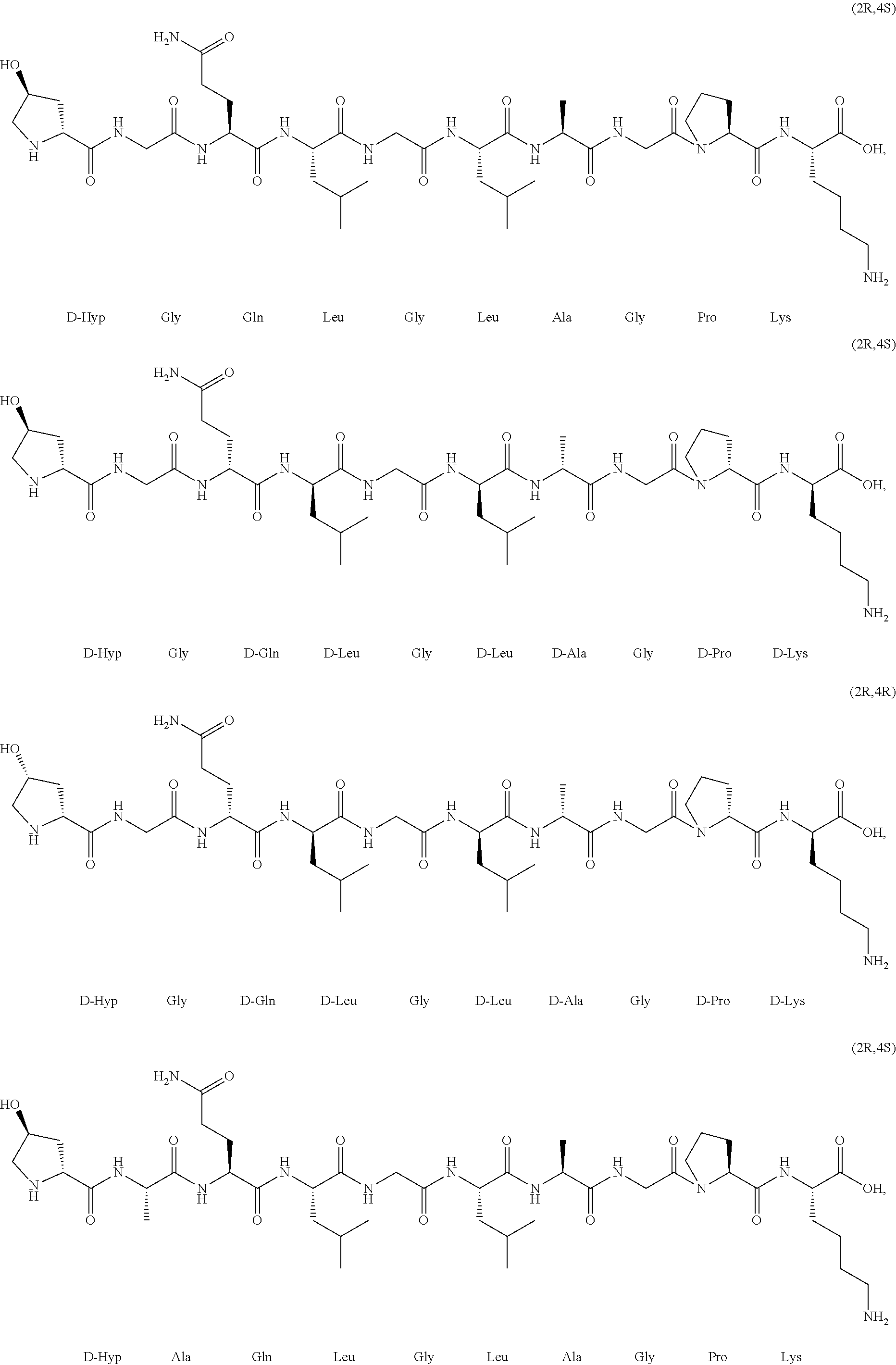

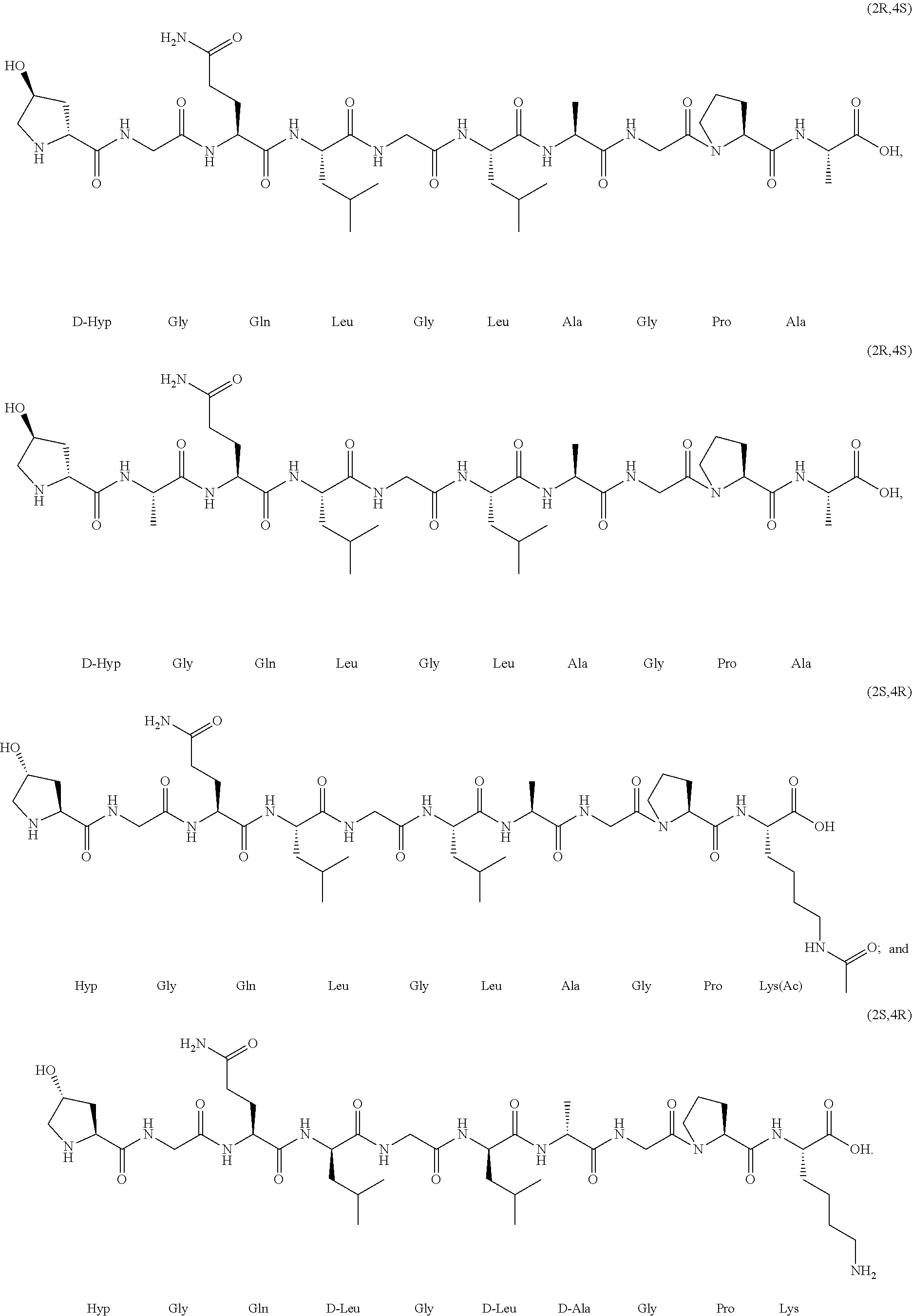

The present invention also provides a compound represented by Formula (I):

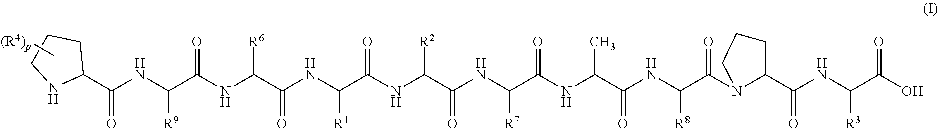

or a pharmaceutically acceptable salt thereof;

wherein:

R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl;

R⁴, independently for each occurrence, is selected from substituted or unsubstituted alkyl, aryl, arylalkyl, heterocyclyl, oxo, —OR^{b}, —CH₂OR^{b}, halo, hydroxyl, and hydroxyalkyl;
R^{b} is substituted or unsubstituted alkyl, aryl, arylalkyl, or heterocyclyl;

p is 0, 1, or 2;

R⁶ is hydrogen or substituted or unsubstituted alkyl; and

R⁷, R⁸, and R⁹ are each independently hydrogen or alkyl; wherein at least one of:

(a) at least one of R¹, R², and R³ is substituted or unsubstituted (C₂-C₁₀)haloalkyl;

(b) at least one of alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl is substituted with one or more substituents selected from —R^{a}, —OR^{a}, —SR^{a}, —N(R^{a})₂, —N(R^{a})₃⁺, —NR^{a}, —NHC(═O)R^{c}, —C(═O)R^{c}, —C(═O)N(R^{a})₂, —S(═O)₂R^{c}, —OS(═O)₂OR^{a}, —S(═O)₂OR^{a}, —S(═O)₂N(R^{a})₂, —S(═O)R^{c}, —OP(═O)(OR^{a})₂, -(alkylene)-C(═O)R^{c}, —C(═S)R^{c}, —C(═O)OR^{a}, -(alkylene)-C(═O)OR^{a}, —C(═S)OR^{a}, —C(═O)SR^{a}, —C(═S)SR^{a}, -(alkylene)-C(═O)N(R^{a})₂, —C(═S)N(R^{a})₂, and —C(—NR^{a})N(R^{a})₂; and

at least one occurrence of R^{a} or R^{c} is heterocyclylalkyl, cycloalkyl, or (cycloalkyl)alkyl;

(c) the compound comprises at least one D-amino acid residue; or

(d) at least two occurrences of R^{a};
at least two occurrences of R^{c}; or
at least one occurrence of R^{a} and at least one occurrence of R^{c}; and
at least one occurrence of R^{a} and/or R^{c} differs from the other occurrences.

In certain embodiments, at least one of R¹, R², and R³ is substituted or unsubstituted (C₂-C₁₀)haloalkyl.

In certain embodiments, at least one of alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl is substituted with one or more substituents selected from —R^{a}, OR^{a}, —SR^{a}, —N(R^{a})₂, —N(R^{a})₃⁺, ═NR^{a}, —NHC(═O)R^{c}, —C(═O)R^{c}, —C(═O)N(R^{a})₂, —S(═O)₂R^{c}, —OS(═O)₂OR^{a}, —S(═O)₂OR^{a}, —S(═O)₂N(R^{a})₂, —S(═O)R^{c}, —OP(═O)(OR^{a})₂, -(alkylene)-C(═O)R^{c}, —C(═S)R^{c}, —C(═O)OR^{a}, -(alkylene)-C(═O)OR^{a}, —C(═S)OR^{a}, —C(═O)SR^{a}, —C(═S)SR^{a}, -(alkylene)-C(═O)N(R^{a})₂, —C(═S)N(R^{a})₂, and —C(—NR^{a})N(R^{a})₂; and at least one occurrence of R^{a} or R^{c} is heterocyclylalkyl, cycloalkyl, or (cycloalkyl)alkyl.

In certain embodiments, the compound comprises at least one D-amino acid residue.

In certain embodiments, the compound has:

at least two occurrences of R^{a};

at least two occurrences of R^{c}; or

at least one occurrence of R^{a} and at least one occurrence of R^{c}; and

at least one occurrence of R^{a} and/or R^{c} differs from the other occurrences.

In certain embodiments:

R¹, R², and R³ are each independently H or substituted or unsubstituted alkyl, arylalkyl, or heterocyclylalkyl;

R⁴, independently for each occurrence, is selected from substituted or unsubstituted alkyl, oxo, hydroxyl, —OR^{b}, hydroxyalkyl, —CH₂OR^{b}, and halo;
R^{b} is substituted or unsubstituted alkyl, aryl, arylalkyl, or heterocyclyl;

R⁶ is hydrogen or substituted or unsubstituted alkyl; and

R⁷, R⁸, and R⁹ are each independently hydrogen or alkyl.

In some embodiments, where indicated, alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl is unsubstituted or is substituted with one or more substituents selected from halo, haloalkyl, oxo, —CN, —NO₂, ═N—OH, —N₃, —R^{a}, —OR^{a}, —SR^{a}, —N(R^{a})₂, —N(R^{a})₃⁺, —NR^{a}, —NHC(═O)R^{c}, —C(═O)R^{c}, —C(═O)N(R^{a})₂, —S(═O)₂R^{c}, —OS(═O)₂OR^{a}, —S(═O)₂OR^{a}, —S(═O)₂N(R^{a})₂, —S(═O)R^{c}, —OP(═O)(OR^{a})₂, -(alkylene)-C(═O)R^{c}, —C(═S)R^{c}, —C(═O)OR^{a}, -(alkylene)-C(═O)OR^{a}, —C(═S)OR^{a}, —C(═O)SR^{a}, —C(═S)SR^{a}, -(alkylene)-C(═O)N(R^{a})₂, —C(═S)N(R^{a})₂, and —C(—NR^{a})N(R^{a})₂;

R^{a}, independently for each occurrence, is hydrogen, or substituted or unsubstituted alkyl, aryl, arylalkyl, heterocyclyl, heterocyclylalkyl, cycloalkyl, or (cycloalkyl)alkyl; and

R^{c}, independently for each occurrence, is substituted or unsubstituted alkyl, aryl, arylalkyl, heterocyclyl, heterocyclylalkyl, cycloalkyl, or (cycloalkyl)alkyl.

In more particular embodiments, where indicated, alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl is unsubstituted or is substituted with one or more substituents selected from halo, haloalkyl, oxo, —R^{a}, —OR^{a}, —N(R^{a})₂, —N(R^{a})₃⁺, —NHC(═O)R^{c}, —C(═O)R^{c}, —C(═O)N(R^{a})₂, —C(═O)OR^{a}, -(alkylene)-C(═O)OR^{a}, and -(alkylene)-C(═O)N(R^{a})₂;

R^{a}, independently for each occurrence, is hydrogen, or substituted or unsubstituted alkyl, aryl, arylalkyl, heterocyclyl, heterocyclylalkyl, cycloalkyl, or (cycloalkyl)alkyl; and

R^{c}, independently for each occurrence, is substituted or unsubstituted alkyl, aryl, arylalkyl, heterocyclyl, heterocyclylalkyl, cycloalkyl, or (cycloalkyl)alkyl.

In certain such embodiments, R^{a}, independently for each occurrence, is hydrogen, alkyl, aryl, arylalkyl, heterocyclyl, or heterocyclylalkyl; and R^{c}, independently for each occurrence, is alkyl, aryl, arylalkyl, heterocyclyl, or heterocyclylalkyl.

In certain embodiments, the compound has the structure of formula (I-10L):

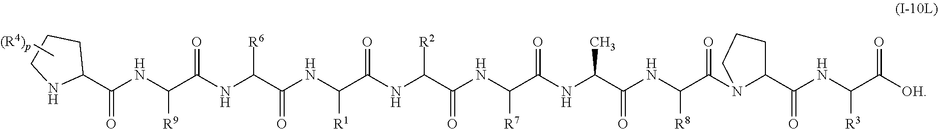

Alternatively, the compound may have the structure of formula (I-10D):

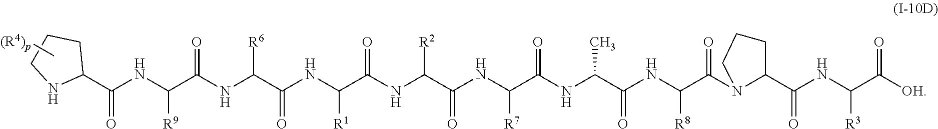

In certain embodiments, R¹ is substituted or unsubstituted (C₂-C₁₀)haloalkyl.

In certain embodiments, R¹ is substituted or unsubstituted alkyl, arylalkyl, or heterocyclylalkyl.

More specifically, R¹ may be selected from substituted or unsubstituted alkyl,

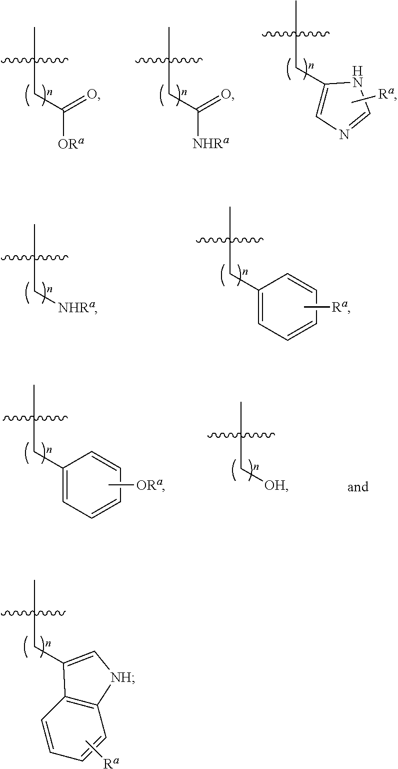

R^{a} is hydrogen or alkyl; and
n is an integer from 1 to 10, preferably 1-5, more preferably 1-3.

Exemplary R¹ groups include

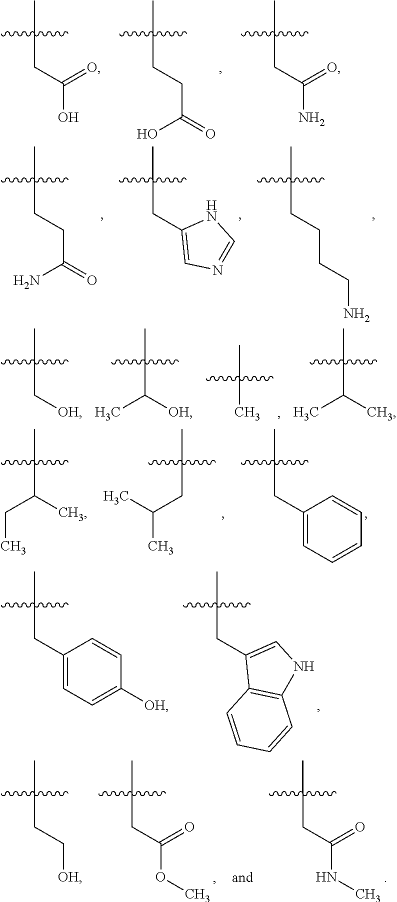

In some preferred embodiments, R¹ is

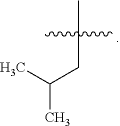 [SMILES: *CC(C)C]

In alternative preferred embodiments, R¹ is

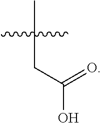 [SMILES: *CC(=O)O]

In certain embodiments, the compound has the structure of formula (I-1L):

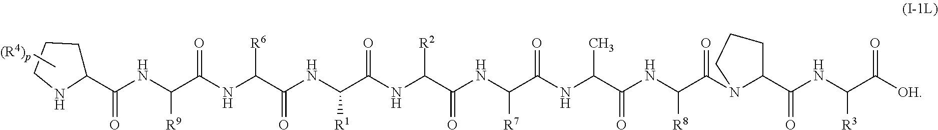

Alternatively, the compound may have the structure of formula (I-1D)

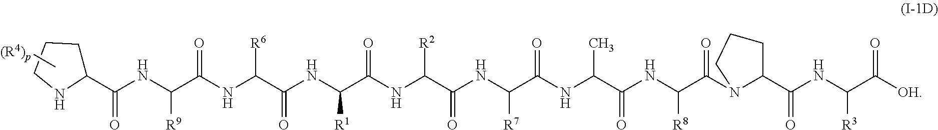

In certain embodiments, R² is substituted or unsubstituted (C₂-C₁₀)haloalkyl.

In certain embodiments, R² is H or substituted or unsubstituted alkyl, arylalkyl, or heterocyclylalkyl.

In some embodiments, R² is selected from hydrogen, substituted or unsubstituted alkyl,

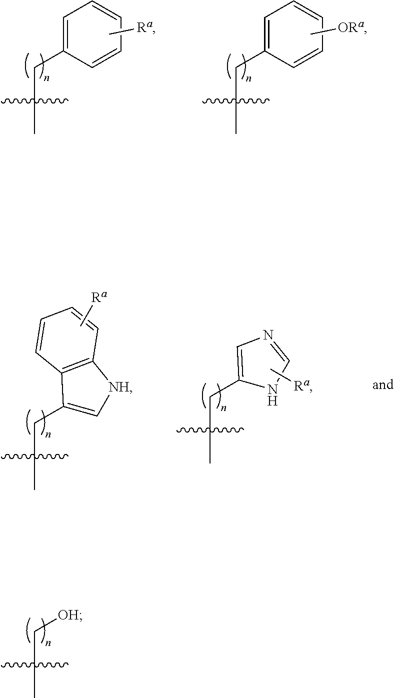

R^{a} is hydrogen or alkyl; and

n is an integer from 1 to 10, preferably 1-5, more preferably 1-3.

Exemplary R² groups include

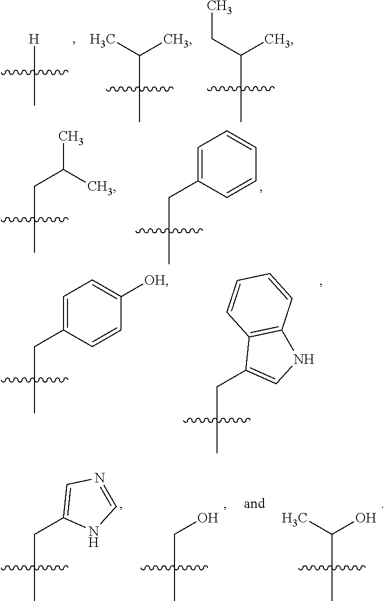

Preferably, R² is hydrogen.

In certain embodiments, the compound has the structure of formula I-2L):

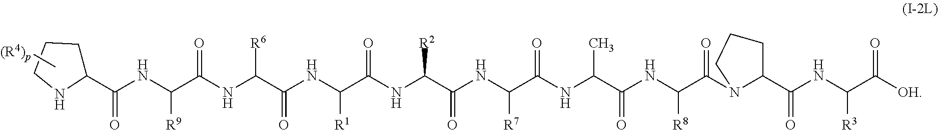

Alternatively, the compound may have the structure of formula (I-2D):

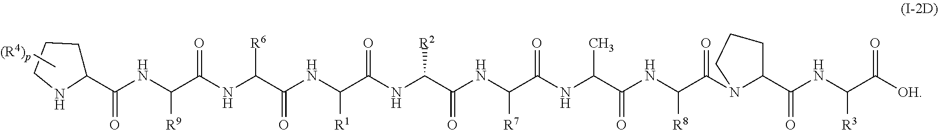

In certain embodiments, R³ is substituted or unsubstituted (C₂-C₁₀)haloalkyl.

In certain embodiments, R³ is substituted or unsubstituted alkyl or arylalkyl.

In some embodiments, R³ is selected from substituted or unsubstituted alkyl,

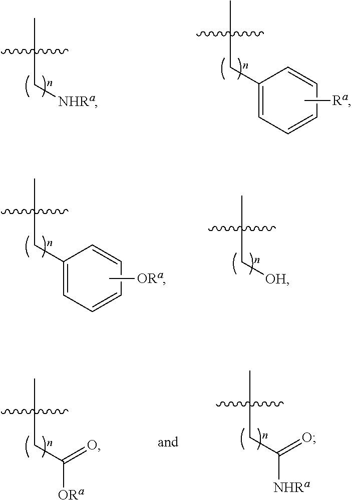

R^{a} is hydrogen or alkyl; and
n is an integer from 1 to 10, preferably 1-5, more preferably 1-3.

Exemplary R³ groups include

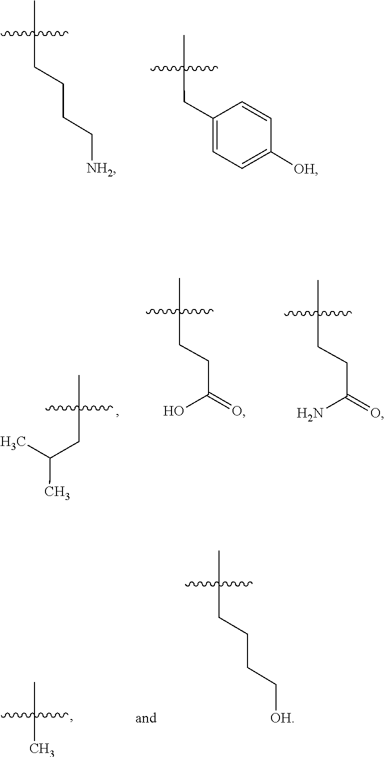

### Preferably, R³ is

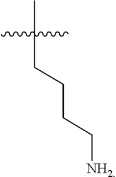 [SMILES: *CCCCN]

In certain embodiments, the compound has the structure of formula (I-3L):

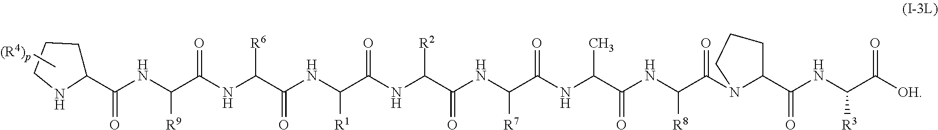

Alternatively, the compound may have the structure of formula (I-3D):

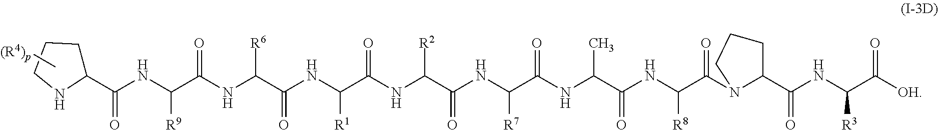

In certain embodiments, p is 1 or 2; and R⁴, independently for each occurrence, is selected from substituted or unsubstituted alkyl, —OR^{b}, —CH₂OR^{b}, halo, hydroxyl, and hydroxyalkyl.

In certain embodiments, p is 1 or 2; and R⁴, independently for each occurrence, is selected from —CH₃, halo, hydroxyl, and hydroxyalkyl.

In certain preferred embodiments, R⁴ is hydroxyl. In alternative preferred embodiments, R⁴ is —CH₃.

In any of the above embodiments, p may be 1.

In certain embodiments, the compound has the structure of formula (I-4Lg):

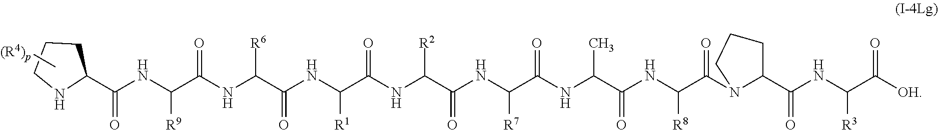

In certain embodiments, the compound has the structure of formula (I-4La):

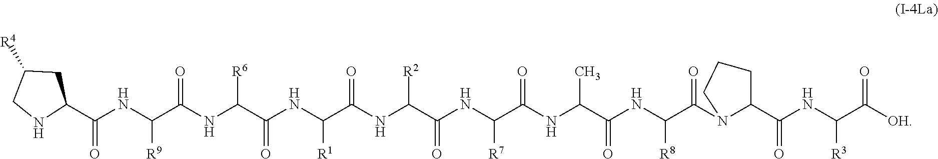

In certain embodiments, the compound has the structure of formula (I-4Lb):

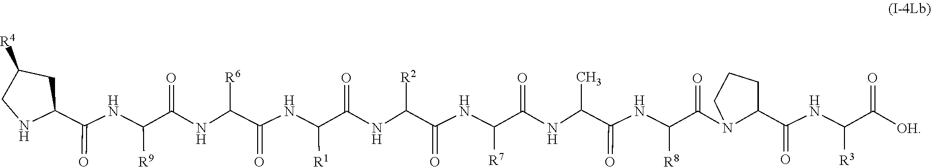

In certain embodiments, the compound has the structure of formula (I-4Lc):

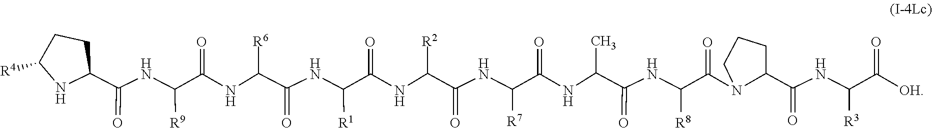

In certain embodiments, the compound has the structure of formula (I-4Lc), provided that R⁴ is not hydroxyl.

In certain embodiments, the compound has the structure of formula (I-4Dg):

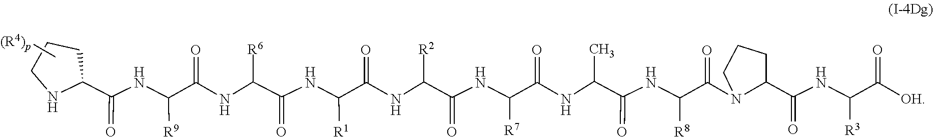

In certain embodiments, the compound has the structure of formula (I-4Da):

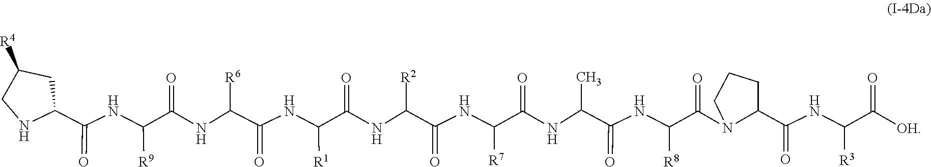

In certain embodiments, the compound has the structure of formula (I-4Db):

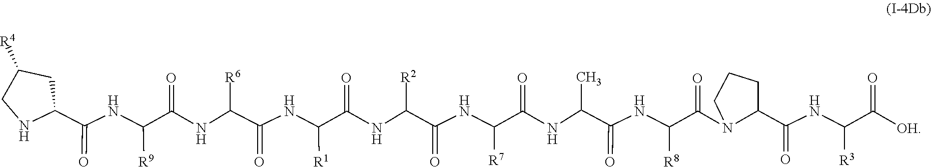

In certain embodiments, the compound has the structure of formula (I-4Dc):

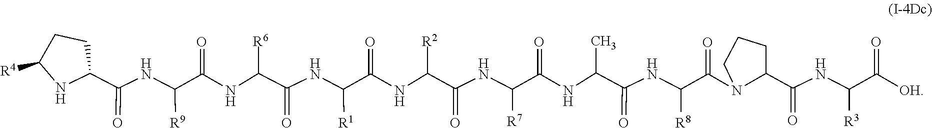

In certain embodiments, the compound has the structure of formula (I-4Dc), provided that R⁴ is not hydroxyl.

In certain embodiments, R⁴ is oxo.

In certain embodiments, the compound has the structure of formula (I-4Ld):

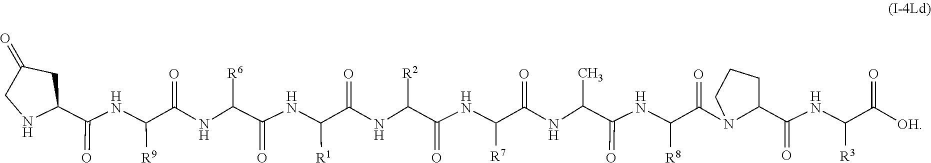

In certain embodiments, the compound has the structure of formula (I-4Le):

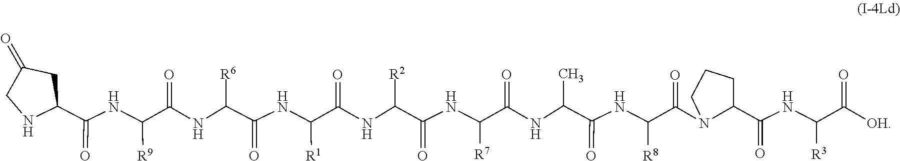

In certain embodiments, the compound has the structure of formula (I-4Dd):

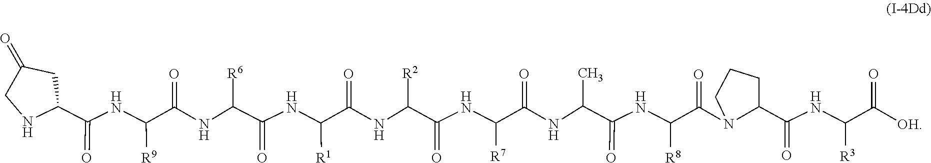

In certain embodiments, the compound has the structure of formula (I-4De):

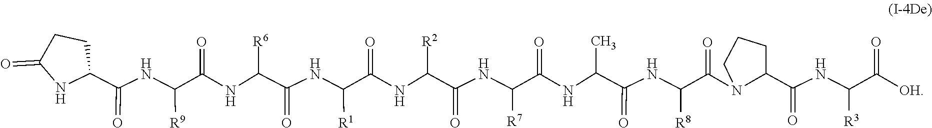

In certain embodiments, R⁶ is hydrogen or alkyl, wherein the alkyl is optionally substituted with one occurrence of —C(═O)NH₂. In certain embodiments, wherein R⁶ is alkyl optionally substituted with one occurrence of —C(═O)NH₂. For example, R⁶ may be —CH₃. Alternatively, R⁶ may be

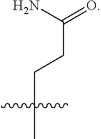 [SMILES: *CCC(N)=O]

In certain embodiments, the compound has the structure of formula (I-6L):

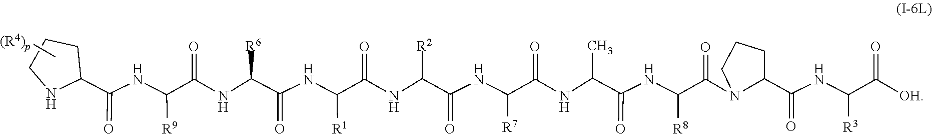

Alternatively, the compound may have the structure of formula (I-6D):

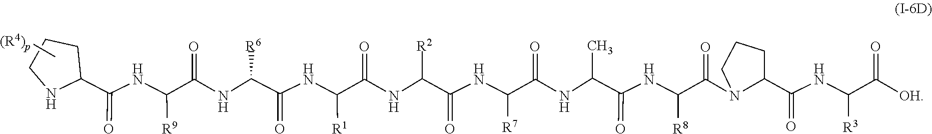

In certain embodiments, R⁷ is (C₁-C₁₀)alkyl, preferably

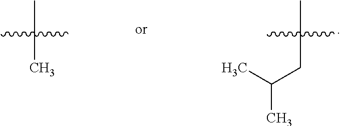

In certain embodiments, the compound has the structure of formula (I-7L):

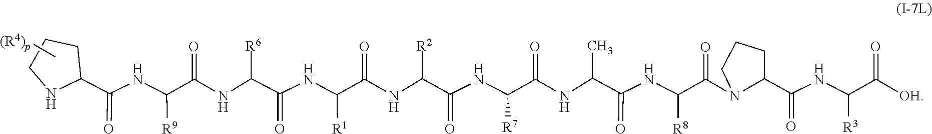

Alternatively, the compound may have the structure of formula (I-7D):

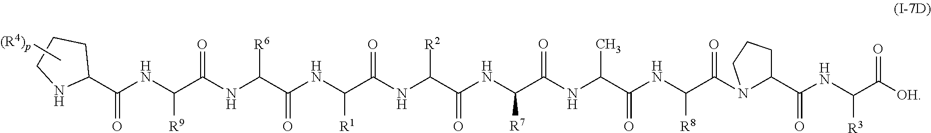

In certain embodiments, the compound has the structure of formula (I-11L):

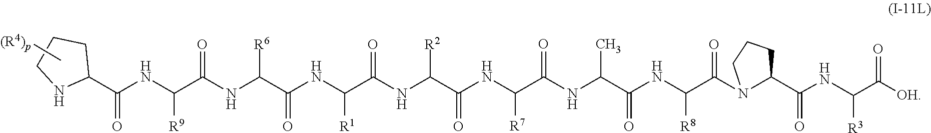

Alternatively, the compound may have the structure of formula (I-11D):

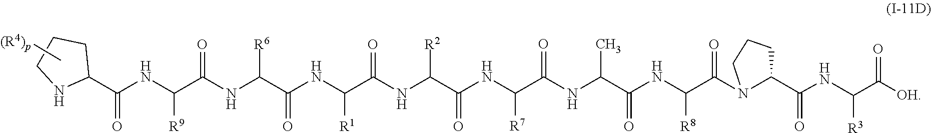

In certain embodiments, R⁸ is —CH₃ or —H, preferably —H.

In certain embodiments, R⁹ is —CH₃ or —H, preferably —H.

In certain embodiments, the compound is

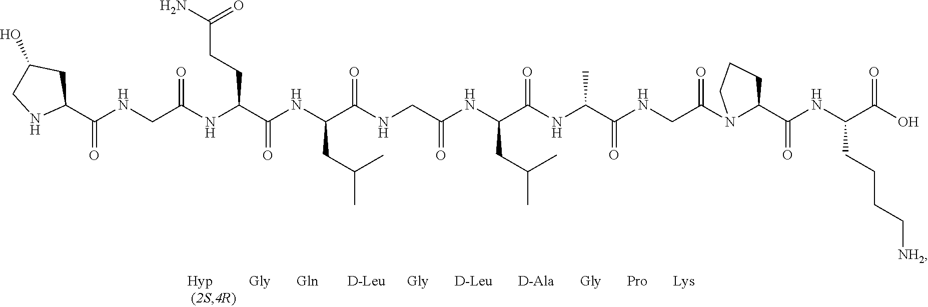 [SMILES: CC(C)C[C@@H](NC(=O)CNC(=O)[C@@H](CC(C)C)NC(=O)[C@H](CCC(N)=O)NC(=O)CNC(=O)[C@@H]1C[C@@H](O)CN1)C(=O)N[C@H](C)C(=O)NCC(=O)N1CCC[C@H]1C(=O)N[C@@H](CCCCN)C(=O)O]

or a pharmaceutically acceptable salt thereof.

In certain embodiments, the compound is a peptide having an amino acid sequence represented by HyP-Gly-Gln-Xaa-Gly-Leu-Ala-Gly-Pro-Lys;

or a pharmaceutically acceptable salt and/or stereoisomer thereof;
wherein Xaa is selected from Glu, Asn, Gln, His, Lys, Ser, Thr, Ala, Val, Ile, Leu, Phe, Tyr, Trp, homo-Ser, Asp(Me), and Asn(Me).

In certain such embodiments, at least one, at least two, at least three, at least four, at least five, at least six, or at least seven amino acid residues in the peptide are D-amino acid residues.

The peptide may be a variant of a collagen type II α1-derived peptide. The collagen type II α1 may be isolated from the extracellular matrix derived from animal chondrocytes.

The term “peptide” used in the present invention refers to a compound in which two or more amino acids are linked by a peptide bond. Further, it is classified into dipeptide, tripeptide, tetrapeptide, and the like according to the number of constituent amino acids. An oligopeptide has about 10 or fewer peptide bonds, and a polypeptide has a plurality of peptide bonds. In addition, a peptide in the present invention includes a mutated peptide in which its amino acid residue is substituted.

The term “HyP” used in the present invention refers to an amino acid called hydroxyproline, in which a hydroxyl group (—OH) is bonded to the carbon atom at the 4-position of proline. HyP has a structure of C₅H₉NO₃ and may be depicted as follows:

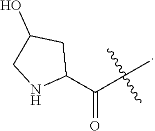

HyP may include all isomers. In addition, HyP may be an isomer represented by the stereochemistry of “2S,4R” unless otherwise specified.

The term “homo-Ser” used in the present invention is called homoserine and refers to an α-amino acid having a hydroxyl group in the side chain. Homo-Ser is an intermediate present in the biosynthesis of threonine and methionine in microorganisms and plants. Homo-Ser may be depicted as follows:

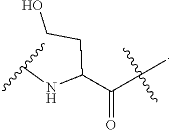

The term “Asp(Me)” used in the present invention indicates an amino acid in which the hydrogen atom of the hydroxyl group (OH) bonded to the carbon atom at the 4-position of aspartic acid is substituted by a methyl group (CH₃). Asp(Me) may be depicted as follows:

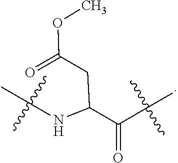

The term “Asn(Me)” used in the present invention indicates an amino acid in which the hydrogen atom of the amine group (NH₂) bonded to the carbon atom at the 4-position of asparagine is substituted by a methyl group (CH₃). Asn(Me) may be depicted as follows:

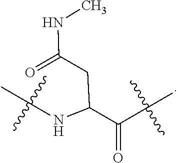

The term “(N-Me)Gly” used in the present invention indicates an amino acid in which the hydrogen atom of the amine group (NH₂) bonded to the carbon atom at the 2-position of glycine is replaced by a methyl group (CH₃). (N-Me)Gly may be depicted as follows:

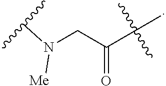

In certain embodiments, the compound is a peptide having an amino acid sequence represented by HyP-Gly-Gln-Asp-Xaa-Leu-Ala-Gly-Pro-Lys;

or a pharmaceutically acceptable salt and/or stereoisomer thereof;
wherein Xaa is selected from Val, Ile, Leu, Ala, Phe, Tyr, Trp, Ser, Thr, and (N-Me)Gly.

In certain such embodiments, at least one, at least two, at least three, at least four, at least five, at least six, or at least seven amino acid residues in the peptide are D-amino acid residues.

In certain embodiments, the compound is a peptide having an amino acid sequence represented by HyP-Gly-Gln-Leu-Gly-Leu-Ala-Gly-Pro-Xaa;

or a pharmaceutically acceptable salt and/or stereoisomer thereof;
wherein Xaa is selected from Tyr, Leu, Glu, Gln, Ala, and Nle(6-OH).

In certain such embodiments, at least one, at least two, at least three, at least four, at least five, at least six, or at least seven amino acid residues in the peptide are D-amino acid residues.

In certain embodiments, the compound is a peptide having an amino acid sequence represented by Xaa-Gly-Gln-Leu-Gly-Leu-Ala-Gly-Pro-Lys;

or a pharmaceutically acceptable salt and/or stereoisomer thereof;
wherein Xaa is selected from:

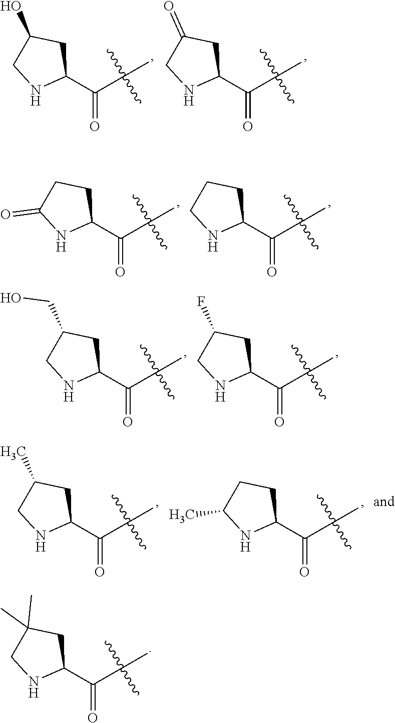

In certain such embodiments, at least one, at least two, at least three, at least four, at least five, at least six, or at least seven amino acid residues in the peptide are D-amino acid residues.

In certain embodiments, the invention provides a compound having the following structure:

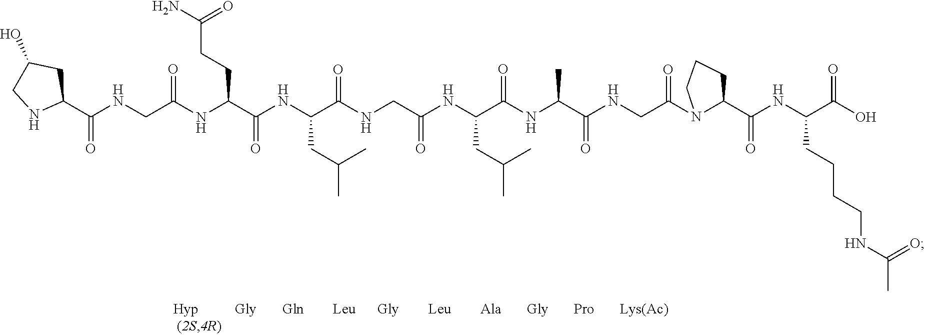 [SMILES: CC(=O)NCCCC[C@H](NC(=O)[C@@H]1CCCN1C(=O)CNC(=O)[C@H](C)NC(=O)[C@H](CC(C)C)NC(=O)CNC(=O)[C@H](CC(C)C)NC(=O)[C@H](CCC(N)=O)NC(=O)CNC(=O)[C@@H]1C[C@@H](O)CN1)C(=O)O]

or a pharmaceutically acceptable salt thereof.

In certain embodiments, the invention provides a compound represented by Formula (V):

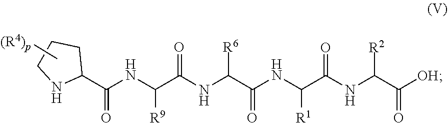

or a pharmaceutically acceptable salt thereof;

wherein:

R¹ and R² are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl;

R⁴, independently for each occurrence, is selected from substituted or unsubstituted alkyl, aryl, arylalkyl, heterocyclyl, oxo, —OR^{b}, —CH₂OR^{b}, halo, hydroxyl, and hydroxyalkyl;
R^{b} is substituted or unsubstituted alkyl, aryl, arylalkyl, or heterocyclyl;

p is 0, 1, or 2;

R⁶ is hydrogen or substituted or unsubstituted alkyl; and

R⁹ is hydrogen or alkyl.

In certain embodiments, R¹ and R² are each independently H or substituted or unsubstituted alkyl;

R⁴ for each occurrence is hydroxyl;

p is 1;

R⁶ is alkyl optionally substituted with one occurrence of —C(═O)NH₂; and

R⁹ is hydrogen.

In certain embodiments, R¹ is substituted or unsubstituted alkyl, such as

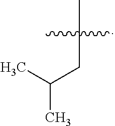

In certain embodiments, the compound has the structure of formula (V-1L)

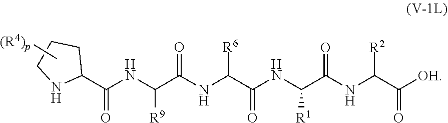

Alternatively, the compound may have the structure of formula (V-1D)

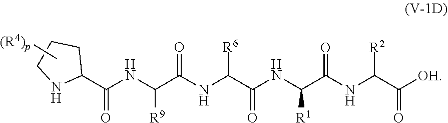

In certain embodiments, R² is H.

In certain embodiments, p is 1 and R⁴ is hydroxyl.

In certain embodiments, the compound has the structure of formula (V-4La):

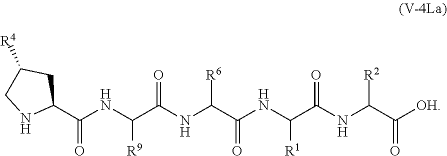

In certain embodiments, the compound has the structure of formula (V-4Lb):

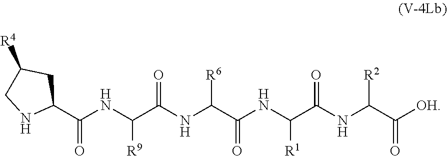

In certain embodiments, the compound has the structure of formula (V-4Da):

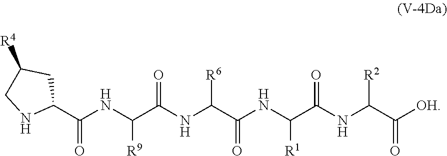

In certain embodiments, the compound has the structure of formula (V-4Db):

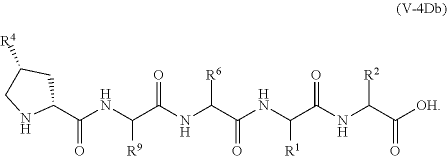

In certain embodiments, R⁶ is alkyl substituted with one occurrence of —C(═O)NH₂, such as

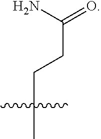 [SMILES: *CCC(N)=O]

In certain embodiments, the compound has the structure of formula (V-6L):

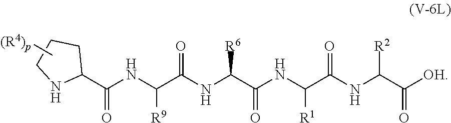

Alternatively, the compound may have the structure of formula (V-6D):

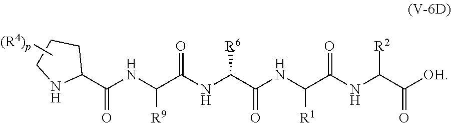

In certain embodiments, R⁹ is —H.

In certain embodiments, the compound is selected from the following:

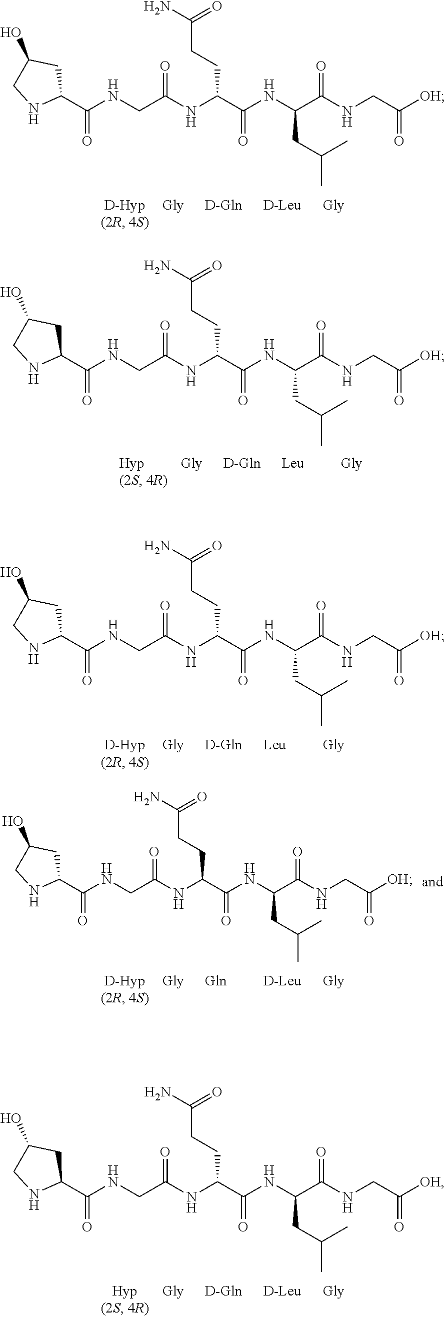 [SMILES: CC(C)C[C@@H](NC(=O)[C@@H](CCC(N)=O)NC(=O)CNC(=O)[C@H]1C[C@H](O)CN1)C(=O)NCC(=O)O ; CC(C)C[C@H](NC(=O)[C@@H](CCC(N)=O)NC(=O)CNC(=O)[C@@H]1C[C@@H](O)CN1)C(=O)NCC(=O)O ; CC(C)C[C@H](NC(=O)[C@@H](CCC(N)=O)NC(=O)CNC(=O)[C@H]1C[C@H](O)CN1)C(=O)NCC(=O)O ; CC(C)C[C@@H](NC(=O)[C@H](CCC(N)=O)NC(=O)CNC(=O)[C@H]1C[C@H](O)CN1)C(=O)NCC(=O)O ; CC(C)C[C@@H](NC(=O)[C@@H](CCC(N)=O)NC(=O)CNC(=O)[C@@H]1C[C@@H](O)CN1)C(=O)NCC(=O)O]

or a pharmaceutically acceptable salt thereof.

In certain embodiments, the invention provides a compound represented by Formula (VI):

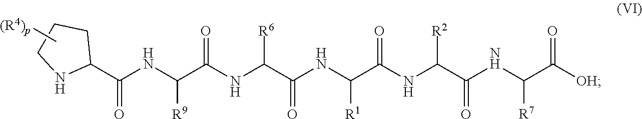

or a pharmaceutically acceptable salt thereof;

wherein:

R¹ and R² are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl

R⁴, independently for each occurrence, is selected from substituted or unsubstituted alkyl, aryl, arylalkyl, heterocyclyl, oxo, —OR^{b}, —CH₂OR^{b}, halo, hydroxyl, and hydroxyalkyl;
R^{b} is substituted or unsubstituted alkyl, aryl, arylalkyl, or heterocyclyl;

p is 0, 1, or 2;

R⁶ is hydrogen or substituted or unsubstituted alkyl;

R⁷ is hydrogen or alkyl; and

R⁹ is hydrogen or alkyl.

In certain embodiments, the invention provides a compound represented by Formula (VI), wherein the compound is not:

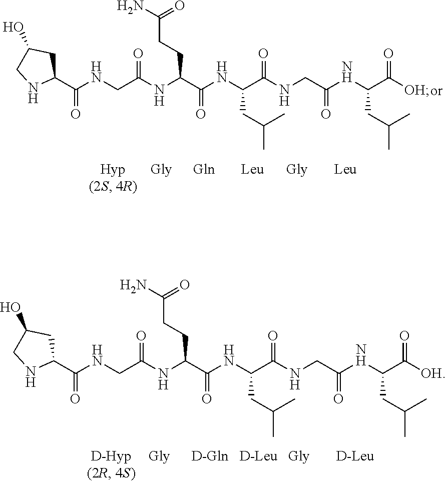 [SMILES: CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)O ; CC(C)C[C@H](NC(=O)CNC(=O)[C@H](CC(C)C)NC(=O)[C@H](CCC(N)=O)NC(=O)CNC(=O)[C@H]1C[C@H](O)CN1)C(=O)O]

In certain embodiments:

R¹ and R² are each independently H or substituted or unsubstituted alkyl;

R⁴ for each occurrence is hydroxyl;

p is 1;

R⁶ is alkyl optionally substituted with one occurrence of —C(═O)NH₂; and

R⁹ is hydrogen.

In certain embodiments, R¹ is substituted or unsubstituted alkyl, such as

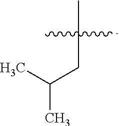 [SMILES: *CC(C)C]

In certain embodiments, the compound has the structure of formula (VI-1L)

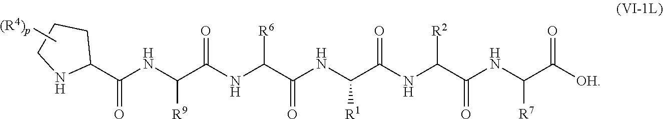

Alternatively, the compound may have the structure of formula (VI-1D)

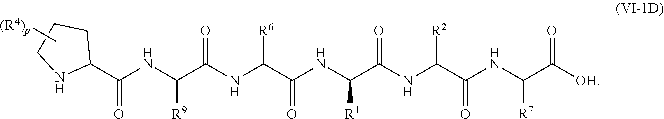

In certain embodiments, R² is H.

In certain embodiments, p is 1 and R⁴ is hydroxyl.

In certain embodiments, the compound has the structure of formula (VI-4La):

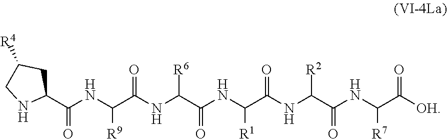

In certain embodiments, the compound has the structure of formula (VI-4Lb):

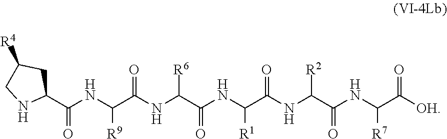

In certain embodiments, the compound has the structure of formula (VI-4Da):

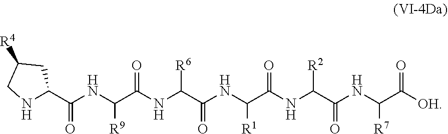

In certain embodiments, the compound has the structure of formula (VI-4Db):

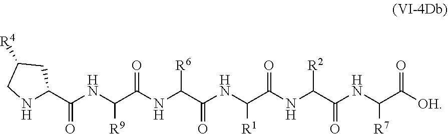

In certain embodiments, R⁶ is alkyl substituted with one occurrence of —C(═O)NH₂, such as

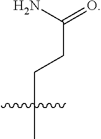 [SMILES: *CCC(N)=O]

In certain embodiments, the compound has the structure of formula (VI-6L):

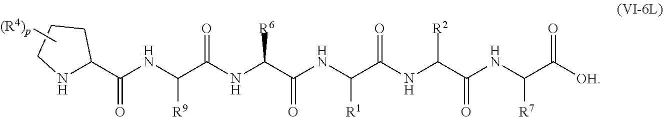

Alternatively, the compound may have the structure of formula (VI-6D):

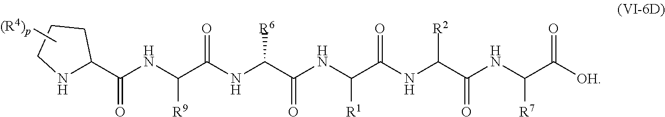

In certain embodiments, R⁹ is —H.

In certain embodiments, R⁷ is (C₁-C₁₀)alkyl, such as

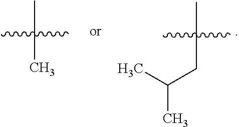

In certain embodiments, the compound has the structure of formula (VI-7L):

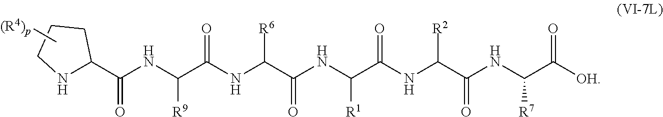

Alternatively, the compound may have the structure of formula (VI-7D):

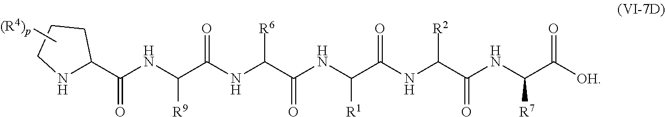

In certain embodiments, the invention provides a compound represented by Formula (VII):

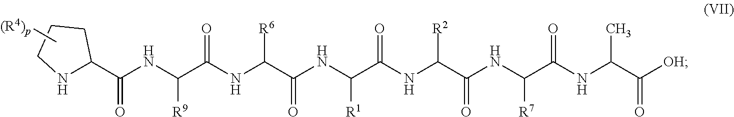

or a pharmaceutically acceptable salt thereof;

wherein:

R¹ and R² are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl;

R⁴, independently for each occurrence, is selected from substituted or unsubstituted alkyl, aryl, arylalkyl, heterocyclyl, oxo, —OR^{b}, —CH₂OR^{b}, halo, hydroxyl, and hydroxyalkyl;
R^{b} is substituted or unsubstituted alkyl, aryl, arylalkyl, or heterocyclyl;

p is 0, 1, or 2;

R⁶ is hydrogen or substituted or unsubstituted alkyl;

R⁷ is hydrogen or alkyl; and

R⁹ is hydrogen or alkyl.

In certain embodiments:

R¹ and R² are each independently H or substituted or unsubstituted alkyl;

R⁴ for each occurrence is hydroxyl;

p is 1;

R⁶ is alkyl optionally substituted with one occurrence of —C(═O)NH₂; and

R⁹ is hydrogen.

In certain embodiments, R¹ is substituted or unsubstituted alkyl, such as

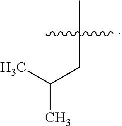 [SMILES: *CC(C)C]

In certain embodiments, the compound has the structure of formula (VII-1L)

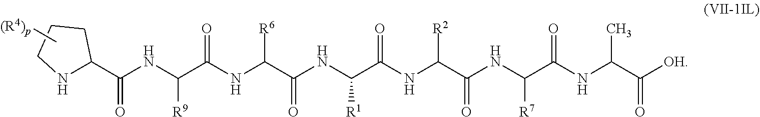

Alternatively, the compound may have the structure of formula (VII-1D)

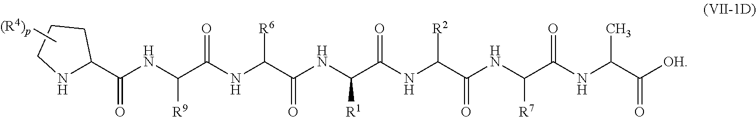

In certain embodiments, R² is H.

In certain embodiments, p is 1 and R⁴ is hydroxyl.

In certain embodiments, the compound has the structure of formula (VII-4La):

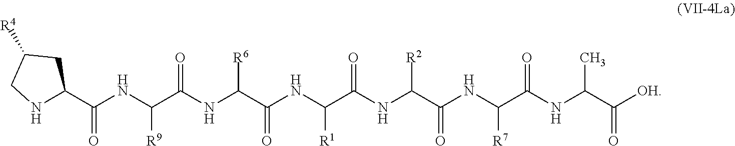

In certain embodiments, the compound has the structure of formula (VII-4Lb):

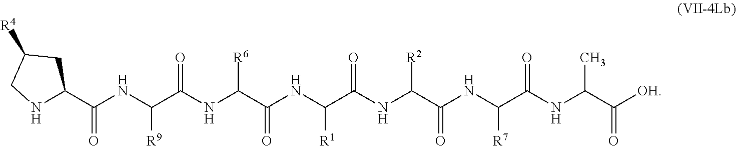

In certain embodiments, the compound has the structure of formula (VII-4Da):

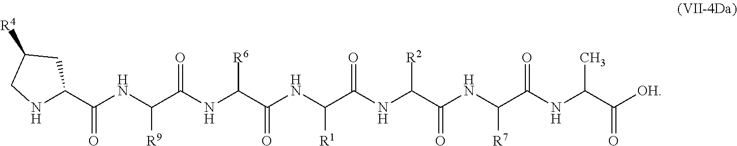

In certain embodiments, the compound has the structure of formula (VII-4Db):

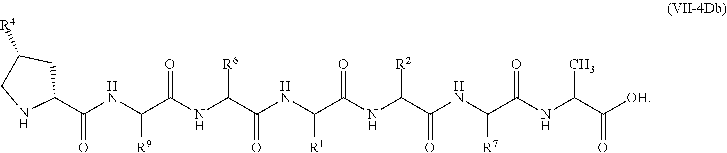

In certain embodiments, R⁶ is alkyl substituted with one occurrence of —C(═O)NH₂, such as

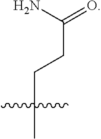 [SMILES: *CCC(N)=O]

In certain embodiments, the compound has the structure of formula (VII-6L):

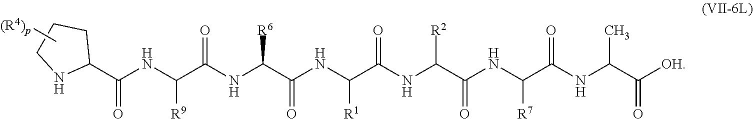

Alternatively, the compound may have the structure of formula (VII-6D):

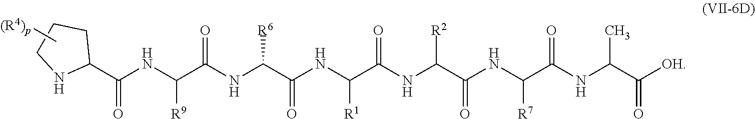

In certain embodiments, R⁹ is —H.

In certain embodiments, R⁷ is (C₁-C₁₀)alkyl, such as

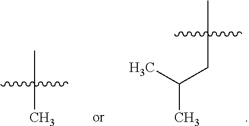

In certain embodiments, the compound has the structure of formula (VII-7L):

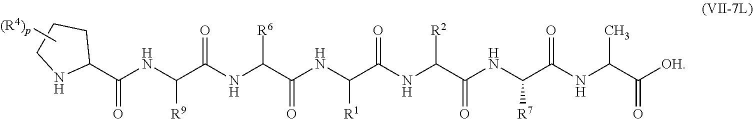

Alternatively, the compound may have the structure of formula (VII-7D):

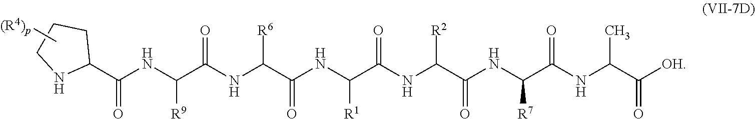

In certain embodiments, the compound has the structure of formula (VII-10L):

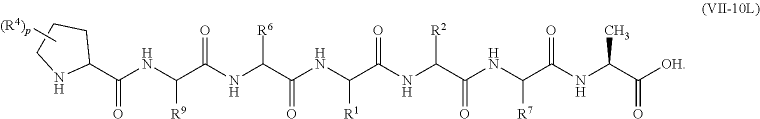

Alternatively, the compound may have the structure of formula VII-10D):

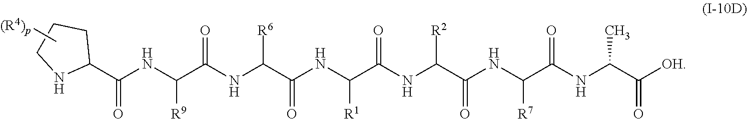

The invention also provides a salt of a compound represented by Formula 8:

[Formula 8]; and

a salt of a compound represented by Formula 10:

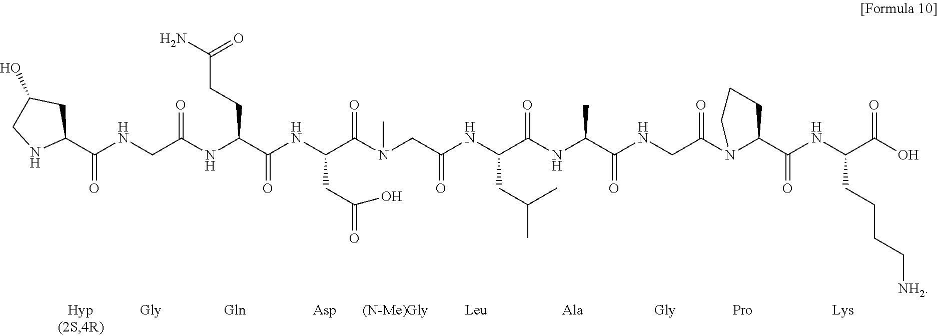 [SMILES: CC(C)C[C@H](NC(=O)CN(C)C(=O)[C@H](CC(=O)O)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O]

In certain embodiments, the compound may be a prodrug, e.g., wherein a hydroxyl in the parent compound is presented as an ester or a carbonate, a carboxylic acid present in the parent compound is presented as an ester, or an amino group is presented as an amide. In certain such embodiments, the prodrug is metabolized to the active parent compound in vivo (e.g., the ester is hydrolyzed to the corresponding hydroxyl or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee.

The compounds of the invention have more than one stereocenter. Accordingly, the compounds of the invention may be enriched in one or more diastereomers. For example, a compound of the invention may have greater than 30% de, 40% de, 50% de, 60% de, 70% de, 80% de, 90% de, or even 95% or greater de. In certain embodiments, the compounds of the invention have substantially one isomeric configuration at one or more stereogenic centers, and have multiple isomeric configurations at the remaining stereogenic centers.

In certain embodiments, the enantiomeric excess of a given stereocenter in the compound is at least 40% ee, 50% ee, 60% ee, 70% ee, 80% ee, 90% ee, 92% ee, 94% ee, 95% ee, 96% ee, 98% ee or greater ee.

As used herein, single bonds drawn without stereochemistry do not indicate the stereochemistry of the compound. The compound of formula (I) provides an example of a compound for which no stereochemistry is indicated.

As used herein, hashed or bolded wedge bonds indicate absolute stereochemical configuration.

In certain embodiments, a therapeutic preparation of the compound of the invention may be enriched to provide predominantly one enantiomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, a therapeutic preparation may be enriched to provide predominantly one diastereomer of the compound of the invention. A diastereomerically enriched mixture may comprise, for example, at least 60 mol percent of one diastereomer, or more preferably at least 75, 90, 95, or even 99 mol percent.

### Methods of Treatment

In certain aspect, provided herein are methods of preventing or treating an IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine mediated disease or disorder in a subject, comprising administering to the subject a compound disclosed herein.

In some embodiments, the subject has elevated levels of IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine.

In some embodiments, the IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine mediated disease is an autoimmune disease, an inflammatory disease, or a cancer, such as Acute posterior multifocal placoid pigment epitheliopathy (APMPPE), Agammaglobulinemia, Alopecia Areata, Amyloidosis, Amyotrophic lateral sclerosis (ALS), Aniridia, Ankylosing spondylitis, Anti-GBM/Anti-TBM nephritis, Antiphospholipid syndrome, Autoimmune hepatitis, Autoimmune inner ear disease, Autoimmune Lymphoproliferative Syndrome, Atopic dermatitis, Asthma, Behçet's Disease, Best Disease, Birdshot Chorioretinopathy, Blepharitis, Bronchiolitis, Cancer (Chondrosarcoma, Cervical, Breast, Lung), Capillary Leak Syndrome, Castleman disease, Celiac disease, Chagas disease, Chalazia and Stye, Chandler's syndrome, Cholesteatoma of Middle Ear, Choroideremia, Chronic recurrent multifocal osteomyelitis, Cogan's syndrome, Collagen Induced Arthritis (CIA), Cold agglutinin disease, Cone Rod Dystrophies, Conjunctivitis, Corneal Wound Healing, CREST syndrome, Crohn's disease, Dermatomyositis, Devic's disease (neuromyelitis optica), Discoid lupus, Dry Eye Disease (DED), Dry macular degeneration (Dry AMD), Endometriosis, Eosinophilic esophagitis, Eosinophilic fasciitis, Essential Iris Atrophy, Evan's syndrome, Farmer's Lung, Fibromyalgia, Giant cell arteritis, Giant cell myocarditis, Giant Papillary Conjunctivitis, Glomerulonephritis, Goodpasture's syndrome, Graft-Versus-Host Disease, Granulomatosis with polyangiitis, Graves' disease, Guillain-Barre syndrome, Gyrate Atrophy, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Iritis, Irritant Dermatitis, Juvenile arthritis, Juvenile/Type 1 Diabetes, Juvenile macular degeneration, Juvenile myositis, Juvenile X-linked Retinoschisis, Kawasaki syndrome, Keratitis, Keratoconjunctivitis sicca (Dry Eye), Late-Onset Retinal Degeneration (L-ORD), Lichen planus, Lichen sclerosus, Lupus (SLE), Macular Edema, Meniere's disease, Multiple sclerosis, Myasthenia gravis, Microscopic polyangiitis, Neuropathic Corneal Pain, Neurotropic Keratitis, Ocular Allergy, Ocular Inflammation (uveitis), Ocular Pain, Ocular Neurodegeneration, Optic Nerve Atrophy, Optic neuritis, Oral Submucous Fibrosis, Osteroarthritis (OA), Osteoporosis, Parkinson's disease, Pars Planitis, Pemphigus, Photokeratitis, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Scleritis, Sjogren's syndrome, Stargardt Disease, Stickler Syndrome, Temporal arteritis/Giant cell arteritis, Thyroid Eye Disease, Trachoma, Transverse myelitis, Trichiasis, Ulcerative colitis, Usher Syndrome, Uveitis, Vasculitis, Vitiligo, Viral myocarditis, or Wegener's granulomatosis (Granulomatosis with Polyangiitis (GPA)), Wet macular degeneration, or Wound Healing.

In some preferred embodiments, the IL-1α, IL-1, IL-2, IL-6, IL-8, IL-10, TNF-α, MMP3, CCL-2, CCL-3, CCL-4, Fas and/or TIMP-1 mediated disease is an autoimmune disease, an inflammatory disease, or a cancer, such as Acute posterior multifocal placoid pigment epitheliopathy (APMPPE), Agammaglobulinemia, Alopecia Areata, Amyloidosis, Amyotrophic lateral sclerosis (ALS), Aniridia, Ankylosing spondylitis, Anti-GBM/Anti-TBM nephritis, Antiphospholipid syndrome, Autoimmune hepatitis, Autoimmune inner ear disease, Autoimmune Lymphoproliferative Syndrome, Atopic dermatitis, Asthma, Behçet's Disease, Best Disease, Birdshot Chorioretinopathy, Blepharitis, Bronchiolitis, Cancer (Chondrosarcoma, Cervical, Breast, Lung), Capillary Leak Syndrome, Castleman disease, Celiac disease, Chagas disease, Chalazia and Stye, Chandler's syndrome, Cholesteatoma of Middle Ear, Choroideremia, Chronic recurrent multifocal osteomyelitis, Cogan's syndrome, Collagen Induced Arthritis (CIA), Cold agglutinin disease, Cone Rod Dystrophies, Conjunctivitis, Corneal Wound Healing, CREST syndrome, Crohn's disease, Dermatomyositis, Devic's disease (neuromyelitis optica), Discoid lupus, Dry macular degeneration (Dry AMD), Endometriosis, Eosinophilic esophagitis, Eosinophilic fasciitis, Essential Iris Atrophy, Evan's syndrome, Farmer's Lung, Fibromyalgia, Giant cell arteritis, Giant cell myocarditis, Giant Papillary Conjunctivitis, Glomerulonephritis, Goodpasture's syndrome, Graft-Versus-Host Disease, Granulomatosis with polyangiitis, Graves' disease, Guillain-Barre syndrome, Gyrate Atrophy, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Iritis, Irritant Dermatitis, Juvenile arthritis, Juvenile/Type 1 Diabetes, Juvenile macular degeneration, Juvenile myositis, Juvenile X-linked Retinoschisis, Kawasaki syndrome, Keratitis, Late-Onset Retinal Degeneration (L-ORD), Lichen planus, Lichen sclerosus, Lupus (SLE), Macular Edema, Meniere's disease, Multiple sclerosis, Myasthenia gravis, Microscopic polyangiitis, Neuropathic Corneal Pain, Neurotropic Keratitis, Ocular Allergy, Ocular Inflammation (uveitis), Ocular Pain, Ocular Neurodegeneration, Optic Nerve Atrophy, Optic neuritis, Oral Submucous Fibrosis, Osteroarthritis (OA), Osteoporosis, Parkinson's disease, Pars Planitis, Pemphigus, Photokeratitis, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Scleritis, Sjogren's syndrome, Stargardt Disease, Stickler Syndrome, Temporal arteritis/Giant cell arteritis, Thyroid Eye Disease, Trachoma, Transverse myelitis, Trichiasis, Ulcerative colitis, Usher Syndrome, Uveitis, Vasculitis, Vitiligo, Viral myocarditis, or Wegener's granulomatosis (Granulomatosis with Polyangiitis (GPA)), Wound Healing.

In some preferred embodiments, the disease or disorder is Dry Eye Disease (DED), Inflammatory Bowel Disease, Keratoconjunctivitis sicca (Dry Eye), Osteoporosis, or Rheumatoid arthritis.

In further preferred embodiments, the disease or disorder is Inflammatory Bowel Disease.

In further preferred embodiments, the disease or disorder is Keratoconjunctivitis sicca (Dry Eye).

In further preferred embodiments, the disease or disorder is Osteoporosis.

In further preferred embodiments, the disease or disorder is Rheumatoid arthritis.

In further preferred embodiments, the disease or disorder is Dry Eye Disease (DED)

In a further aspect, provided are methods of reducing production of IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine in cells of a subject, comprising administering to a subject a compound disclosed herein.

In some embodiments, administering the compound reduces the cytokine and/or chemokine levels by at least 30%, at least 50%, or at least 70% compared to the untreated control.

In yet another aspect, provided are methods of reducing NF-κB transcription activity in cells of a subject, comprising administering to a subject a compound disclosed herein.

In some embodiments, the subject is a mammal, such as a mouse or a human, preferably a human.

In another aspect, provided are methods of preventing or treating Keratoconjunctivitis sicca (Dry Eye) in a subject, comprising administering to the subject a compound disclosed herein.

In yet another aspect, provided are methods of preventing or treating Dry Eye Disease (DED) in a subject, comprising administering to the subject a compound disclosed herein.

### Pharmaceutical Compositions

In certain embodiments, the invention provides a pharmaceutical composition comprising a salt or compound of the invention, or a pharmaceutically acceptable salt thereof; and a pharmaceutically acceptable carrier.

In certain embodiments, the pharmaceutical composition is formulated for topical administration to the eye, e.g., as eye drops.

In certain embodiments at least 50%, 60%, 70%, 80%, or 90% of the compound is present as a salt. Preferably, at least 95% of the compound is present as a salt. Even more preferably, at least 99% of the compound is present as a salt.

In certain embodiments, the present invention provides a pharmaceutical preparation suitable for use in a human patient, comprising any salt or compound of the invention, and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

One embodiment of the present invention provides a pharmaceutical kit comprising a salt or compound of the invention, or a pharmaceutically acceptable salt thereof, and optionally directions on how to administer the compound.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In certain preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ (e.g., wheat germ), olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention (e.g., a compound of formula I, V, VI, or VII) or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, oxalic, mandelic and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I, V, VI, or VII. As another example, the salt may comprise less than one inorganic or organic acid molecule per molecule of base, such as two molecules of compound of Formula I, V, VI, or VII per molecule of tartaric acid.

In further embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

### Example 1: Preparation of YDE-093 to YDE-107 Peptides

YDE peptides (YDE-093 to YDE-107), derivatives of the amino acid sequence of the YDE-011 (WO2018/225961), were obtained through the C-terminal modification or fragmentation of YDE-011.

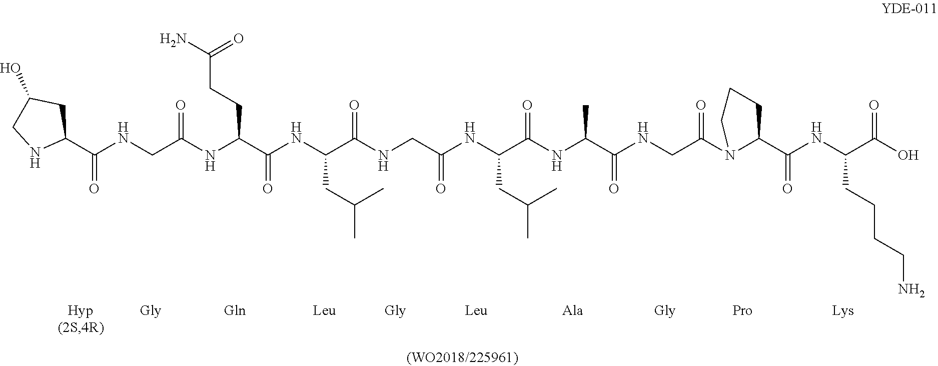 [SMILES: CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O]

In order to prepare C-terminal modified peptide, Fmoc solid-phase peptide synthesis (SPPS) was conducted, based on a standard procedure described by prior invention WO2018/225961 and further a C-terminal amidation reaction was carried out.

YDE-093 to YDE-107 peptides were synthesized by ANYGEN (Gwangju, Korea) and IRBM (Rome, Italy) by substituting one or more different amino acid residues into the peptide (2S,4R)hydroxyproline-GQLGLAGPK(NH-PEG1-NH-Boc) (Table 1).

TABLE 1

| | | | | | | | Solubility |
|---|---|---|---|---|---|---|---|
| | Lot | | | | Purity | Amount | in Water |
| # | No. | Peptide | Structure / Sequence | M. W. | (%) | (mg) | (mg/mL) |
| 1 | K182 143 | YDE- 093 | 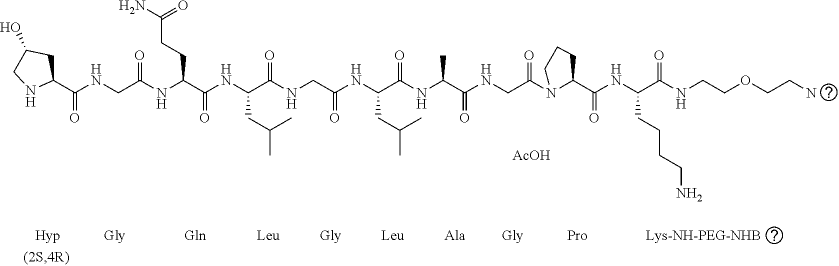 [SMILES: "AcOH" ; CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)NCCOCCN] | 1139.4 | 99.1 | 10 | 10.0 |
| | | | O*GQLGLAGPK(NH-PEG1-NH-Boc) | | | | |
| 2 | K182 472 | YDE- 094 | 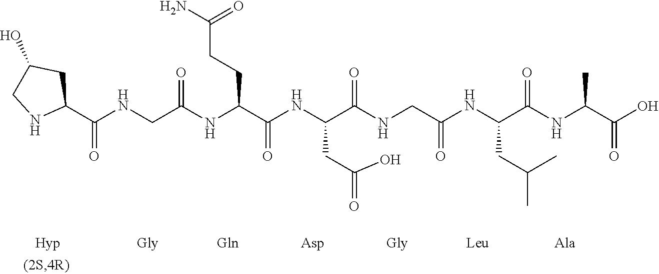 [SMILES: CC(C)C[C@H](NC(=O)CNC(=O)[C@H](CC(=O)O)NC(=O)[C@H](CCC(N)=O)NC(=O)CNC(=O)[C@@H]1C[C@@H](O)CN1)C(=O)N[C@@H](C)C(=O)O] | 672.7 | 97.4 | 30 | 10.0 |
| | | | O*GQDGLA | | | | |
| 3 | I-0417 327- 001- 001 | YDE- 096 | 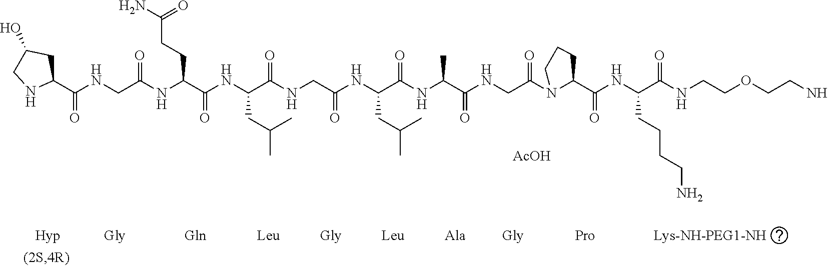 [SMILES: "AcOH" ; CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)NCCOCCN] | 1173.4 | 95.4 | 10 | 10.0 |
| | | | O*GQLGLAGPK(NH-PEG1-NH-Cbz) | | | | |
| 4 | K190 638 | YDE- 100 | 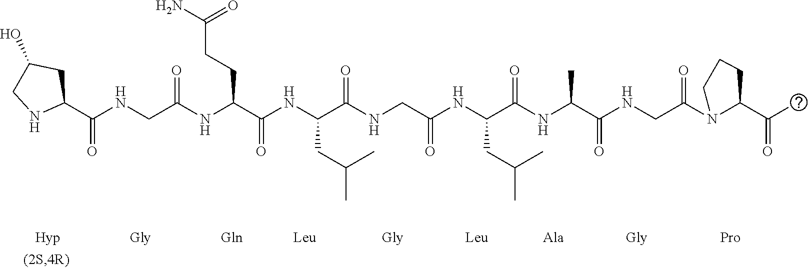 [SMILES: CC(=O)[C@@H]1CCCN1C(=O)CNC(=O)[C@H](C)NC(=O)[C@H](CC(C)C)NC(=O)CNC(=O)[C@H](CC(C)C)NC(=O)[C@H](CCC(N)=O)NC(=O)CNC(=O)[C@@H]1C[C@@H](O)CN1] | 824.9 | 98.7 | 20 | 10.0 |
| | | | O*GQLGLAGP | | | | |
| 5 | K190 639 | YDE- 101 | 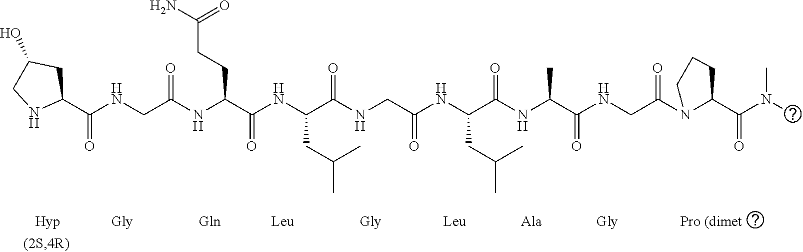 [SMILES: CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N(C)C] | 852.0 | 96.7 | 20 | 10.0 |
| | | | O*GQLGLAGP(dimethyl) | | | | |
| 6 | K190 640 | YDE- 102 | 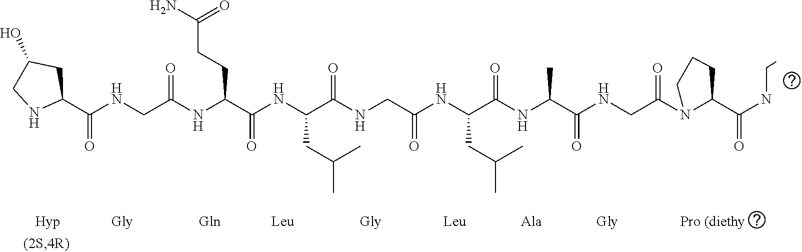 [SMILES: CCNC(=O)[C@@H]1CCCN1C(=O)CNC(=O)[C@H](C)NC(=O)[C@H](CC(C)C)NC(=O)CNC(=O)[C@H](CC(C)C)NC(=O)[C@H](CCC(N)=O)NC(=O)CNC(=O)[C@@H]1C[C@@H](O)CN1] | 880.1 | 97.2 | 20 | 10.0 |
| | | | O*GQLGLAGP(diethyl) | | | | |
| 7 | K190 641 | YDE- 103 | 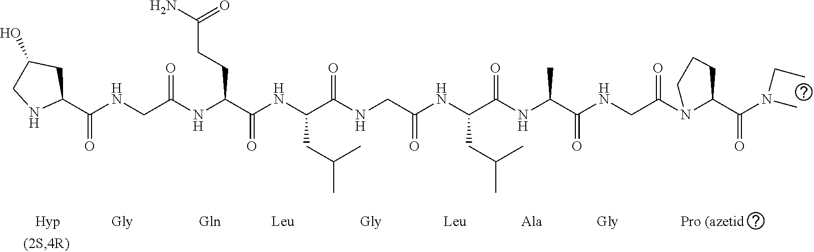 [SMILES: CCN(C)C(=O)[C@@H]1CCCN1C(=O)CNC(=O)[C@H](C)NC(=O)[C@H](CC(C)C)NC(=O)CNC(=O)[C@H](CC(C)C)NC(=O)[C@H](CCC(N)=O)NC(=O)CNC(=O)[C@@H]1C[C@@H](O)CN1] | 864.0 | 97.9 | 20 | 10.0 |
| | | | O*GQLGLAGP(azetidine) | | | | |
| 8 | K190 643 | YDE- 105 | 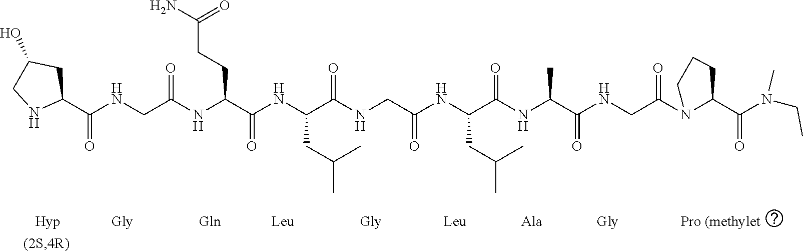 [SMILES: CCN(C)C(=O)[C@@H]1CCCN1C(=O)CNC(=O)[C@H](C)NC(=O)[C@H](CC(C)C)NC(=O)CNC(=O)[C@H](CC(C)C)NC(=O)[C@H](CCC(N)=O)NC(=O)CNC(=O)[C@@H]1C[C@@H](O)CN1] | 866.0 | 97.4 | 20 | 10.0 |
| | | | O*GQLGLAGP(methylethyl) | | | | |
| 9 | K190 644 | YDE- 106 | 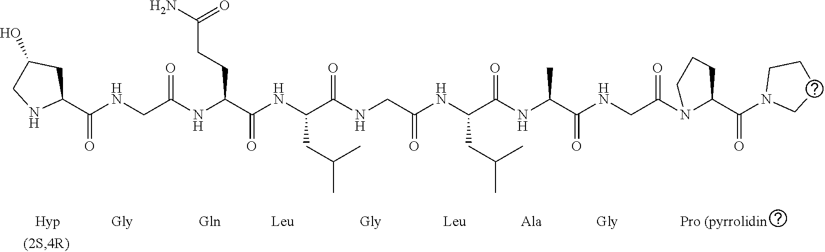 [SMILES: CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N1CCCC1] | 878.0 | 97.2 | 20 | 10.0 |
| | | | O*GQLGLAGP(pyrroidine) | | | | |
| 10 | K190 645 | YDE- 107 | 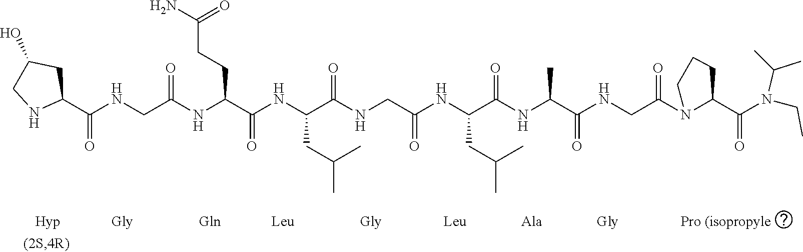 [SMILES: CCN(C(=O)[C@@H]1CCCN1C(=O)CNC(=O)[C@H](C)NC(=O)[C@H](CC(C)C)NC(=O)CNC(=O)[C@H](CC(C)C)NC(=O)[C@H](CCC(N)=O)NC(=O)CNC(=O)[C@@H]1C[C@@H](O)CN1)C(C)C] | 894.1 | 98.2 | 20 | 10.0 |
| | | | O*GQLGLAGP(isopropylethyl) | | | | |

O* = (2S,4R)hydroxyproline

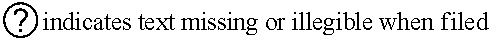

The process for synthesizing the YDE-093 to YDE-107 peptides and the purification procedure thereof conducted by ANYGEN and IRBM are depicted below.

### Analysis of YDE Peptides

The YDE peptides prepared in Example 1 were analyzed by HPLC. As a result, it was confirmed that the purities of YDE-093, YDE-094, YDE-096, YDE-100, YDE-101, YDE-102, YDE-103, YDE-105, YDE-106 and YDE-107 synthesized were 99.1%, 97.4%, 95.4%, 98.7%, 96.7%, 97.2%, 97.9%, 97.4%, 97.2% and 98.2%, respectively.

In addition, the YDE derivatives prepared in Example 1 were analyzed by Ion-Mass. As a result, it was confirmed that the molecular weights of YDE-093, YDE-094, YDE-096, YDE-100, YDE-101, YDE-102, YDE-103, YDE-105, YDE-106 and YDE-107 synthesized were 1139.0, 673.2, 1173.6, 823.9, 851.9, 880.1, 864.5, 866.1, 878.4 and 894.3, respectively.

### Example 2: Evaluation of the Effect of Peptides on Soluble IL-6 Cytokine Release in Poly I:C Stimulated Primary Human Corneal Epithelial Cells

Assay protocol as shown below was used to determine the significant effect of test compounds. The data was statistically analyzed by one-way ANOVA with a Bonferroni post test comparing all the columns. The significance was represented by the p value.

Selected YDE peptides (YDE-093, -096, -100, -101, -102, -103, -104, -105, -106 and -107) were evaluated by assessing the soluble IL-6 cytokine release in poly I:C stimulated primary human corneal epithelial cells by ELISA (Biolegend, 430504).

Specifically, primary corneal epithelial cells (ATCC, ATCC PCS-700-010) were seeded on a 6-well culture plate containing the Corneal Epithelial Cell Basal Medium (ATCC, ATCC PCS-700-030) in the Corneal Epithelial Cell Growth Kit (ATCC, ATCC PCS-700-040) in an amount of 1.2×10⁵ cells per well, which was then cultured for 24 hours under the conditions of 37° C. and 5% CO₂. Then, cells were washed with 1×PBS and replaced with serum free medium which was then cultured for 2 hours under the conditions of 37° C. and 5% CO₂.

After 2 hours, cells were treated with compounds at 6 differential concentrations (Hinokitiol/YDE peptides at 30, 10, 1, 0.1, 0.01, and 0.001 μM) with the DMSO final 0.5% and then cultured for 1 hour under the conditions of 37° C. and 5% CO₂.

After 1 hour, cells were further treated with 25 μg/mL of poly I:C and cultured for 24 hours under the conditions of 37° C. and 5% CO₂.

Following incubation, the supernatant was collected for IL-6 levels was assessed by Sandwich ELISA (Enzyme-linked immunosorbent assay) following manufacturer's instructions. The data was statistically analyzed by one-way ANOVA with a Bonferroni post-test comparing all the columns. The significance was represented by the p value.

### Results & Conclusion

Human corneal epithelial cells on stimulation with Poly I:C resulted in a significantly increase of IL-6 cytokine levels. Reference compound, Hinokitiol was found to significantly attenuate the Poly I:C stimulated IL-6 cytokine release in a dose dependent manner.

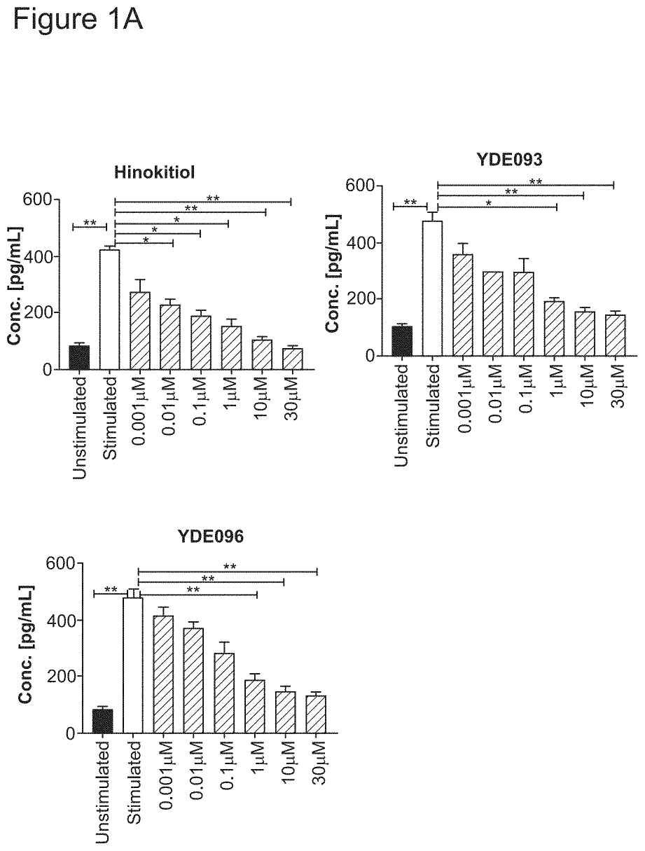

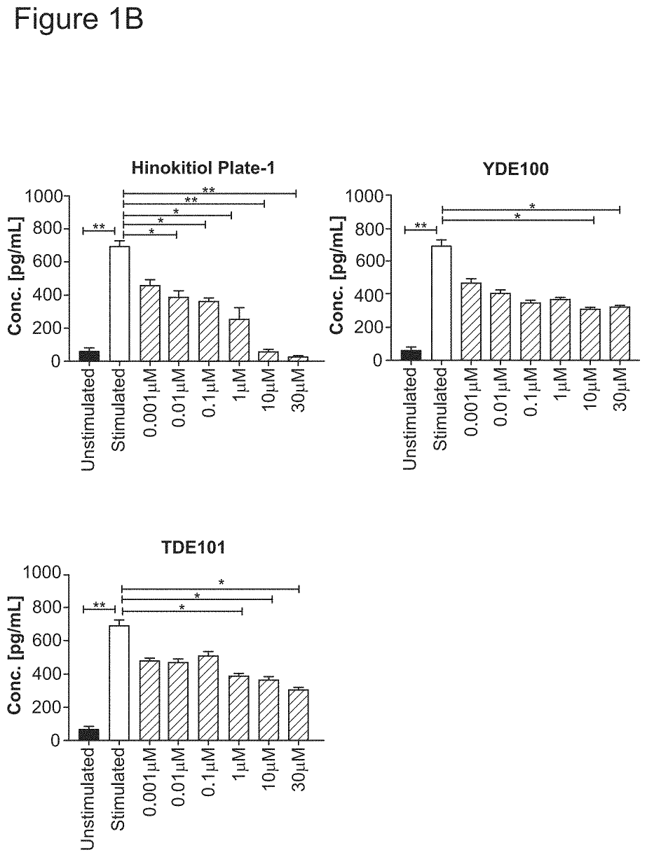

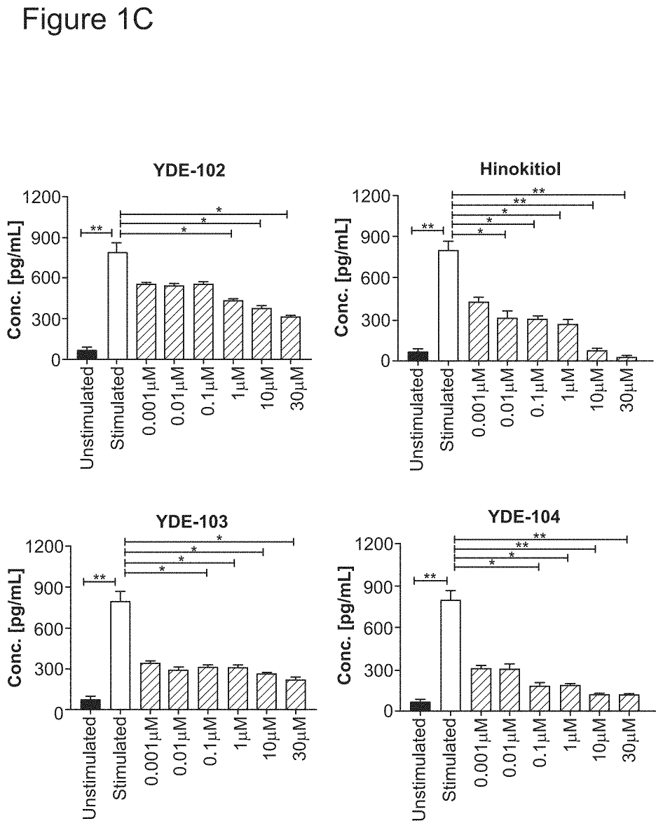

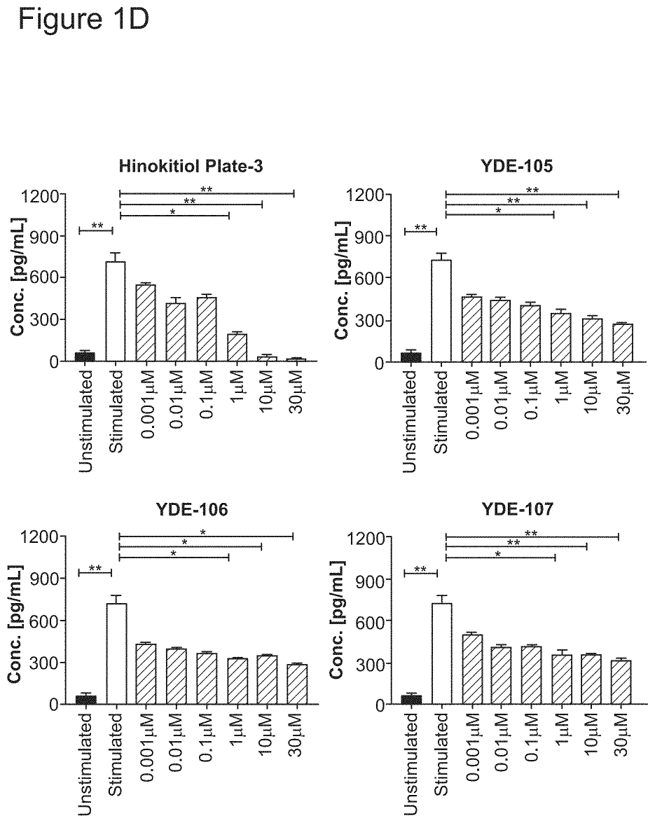

As shown in FIG. 1A-1D, test peptides YDE-093 and YDE-096 were found to attenuate Poly I:C induced of IL-6 cytokine release in a dose dependent manner. Test compounds YDE103, and YDE104, were found to attenuate Poly I:C induced IL-6 cytokine release in a dose dependent manner. This was corroborated by the significant attenuation of IL-6 cytokine release at the tested concentrations that included; 30 μM, 10 μM, 1 μM and 0.1 μM respectively. Test compounds YDE101, YDE102, YDE105, YDE106 and YDE107 were found to attenuate Poly I:C induced IL-6 cytokine release in a dose dependent manner. This was corroborated by the significant attenuation of IL-6 cytokine release at the tested concentrations that included; 30 μM, 10 μM, and 1 μM respectively. Test compound YDE100 was found to attenuate Poly I:C induced IL-6 cytokine release in a dose dependent manner. This was corroborated by the significant attenuation of IL-6 cytokine release at the tested concentrations that included; 30 μM, and 10 μM respectively.

### Example 3: Evaluation of the Effect of Peptides on Cell Proliferation Using Primary Corneal Epithelial Cells

### Study Objective

To determine the cell proliferation assessment of peptides using primary corneal epithelial cells.

### Study Plan

Test System: HCE cells from ATCC.
Assay Format: 96-well plate format
Method of Detection: CellTiter-Glo Luminescent Cell Viability Assay (Pro mega, Cat #G7573)
Assay Controls: Reference compound (hEGF) and untreated cells
Test Samples: 57
Media: Corneal Epithelial Cell Basal Medium+Cell growth kit components EXPERIMENTAL DESIGN

Selected YDE peptides (YDE-012, -019, -038, -044, -045, -047, -048, -049, -050, -051, -052, -053, -054, -055, -056, -057, -058, -059, -060, -061, -062, -063, -064, -065, -066, -067, -072, -073, -074, -075, -076, -077, -078, -079, -080, -081, -082, -083, -084, -085, -086, and -087) were evaluated by assessing the effect of peptides on cell proliferation of primary corneal epithelial cells.

Specifically, 5000 primary corneal epithelial cells/well were seeded in a white opaque 96-well plate and incubated for 24 hrs in a 37° C. incubator supplemented with 5% CO₂. After 24 hours, test compounds were treated at 8 different concentrations. Cells treated with compounds were incubated for 48 & 72 hrs at 37° C. in a 5% CO₂ incubator. Post appropriate incubation time points, CellTiter-Glo luminescent reagent (Promega, Cat #G7573) was added to the plates and incubated at room temperature for 30 min. Luminescence signal was captured using EnVision²¹⁰⁴® Multilabel reader. Assay controls were reference compound (hEGF) and untreated cells.

### Results & Conclusion

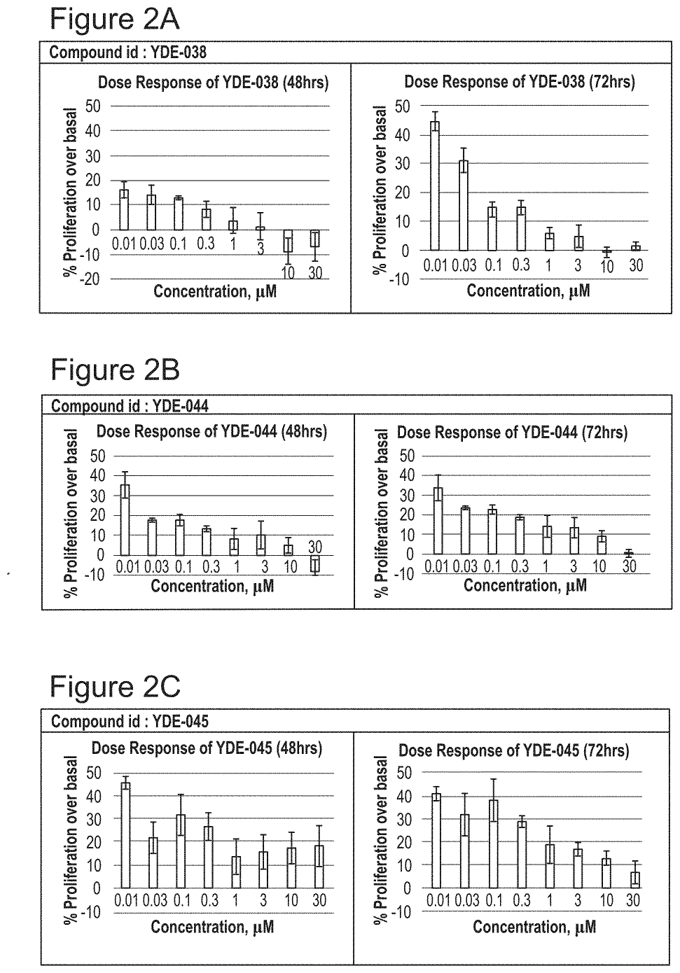

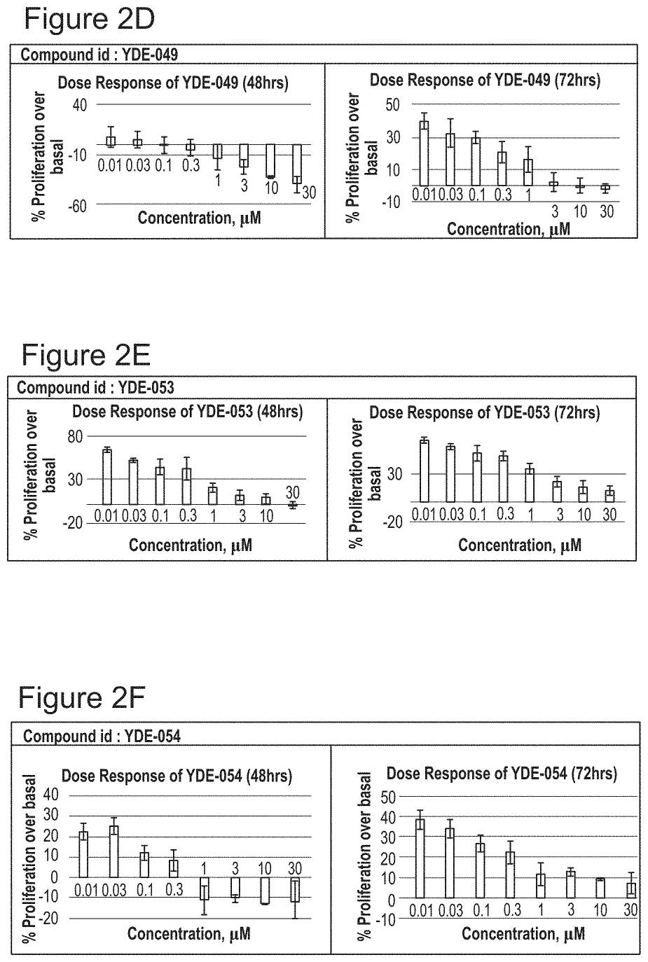

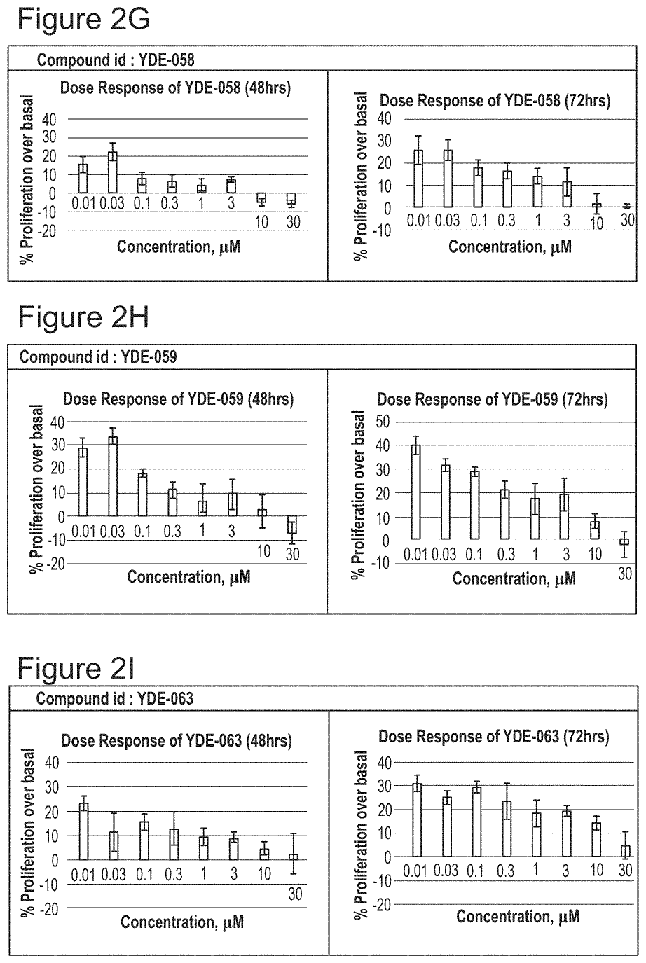

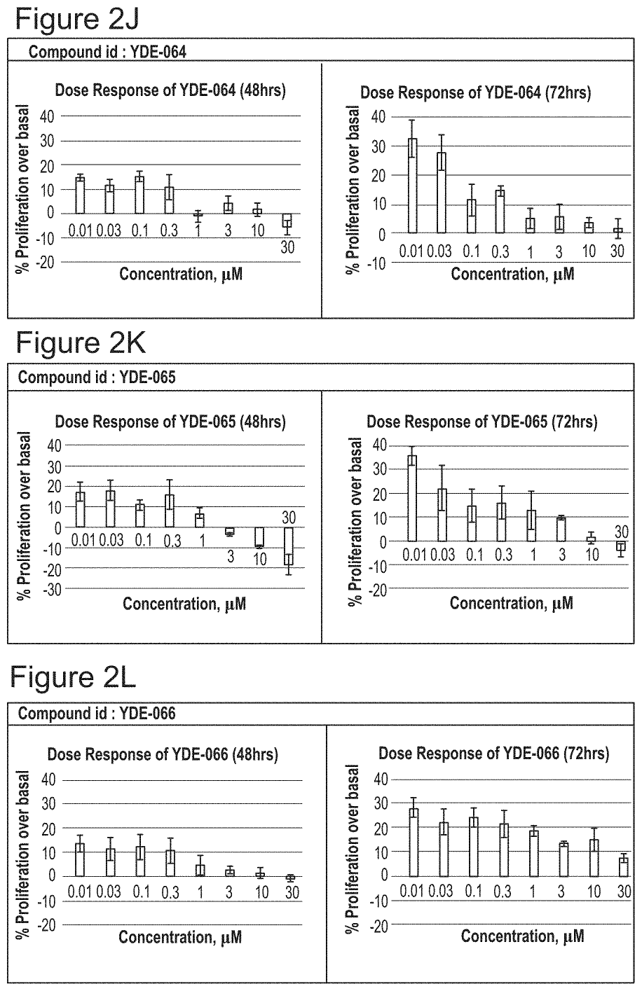

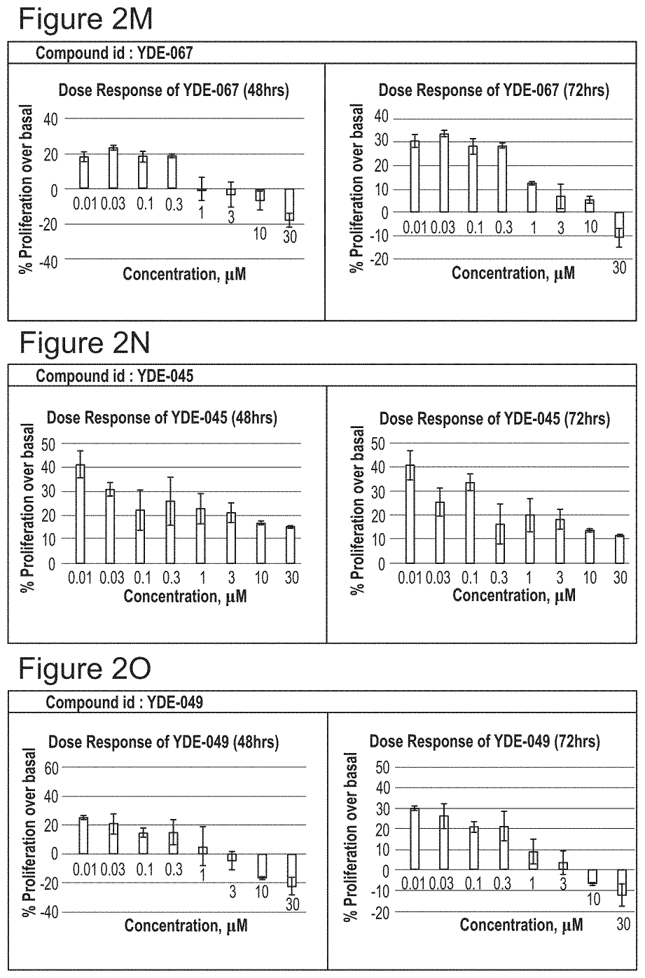

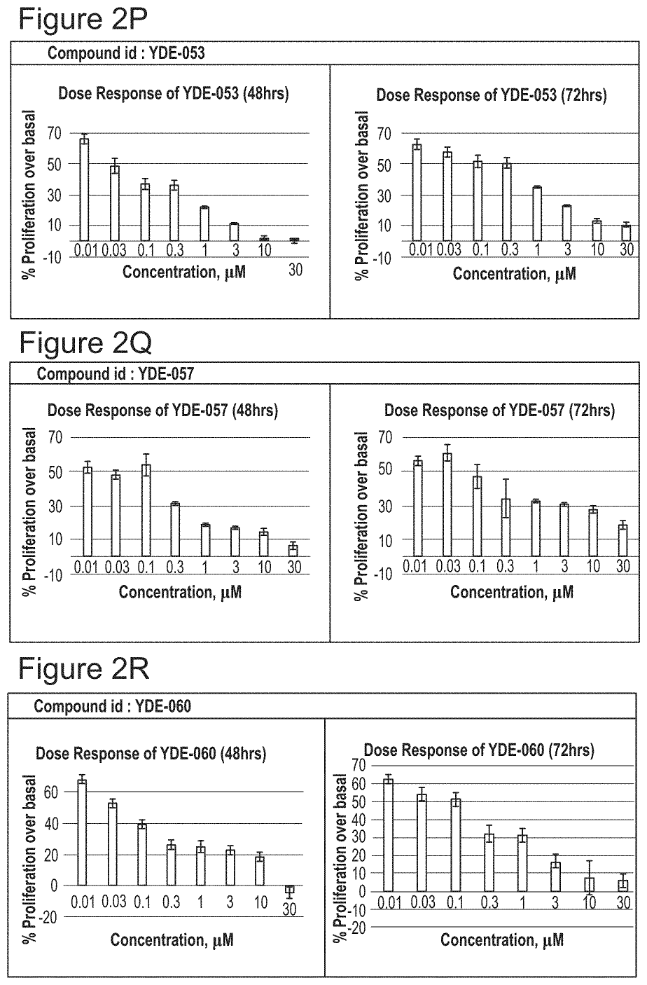

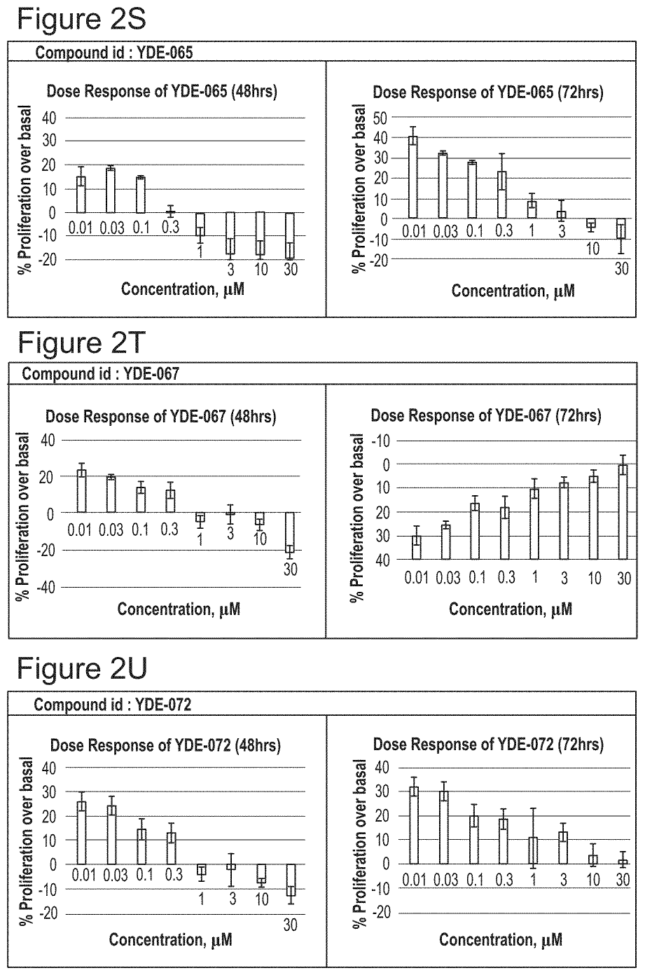

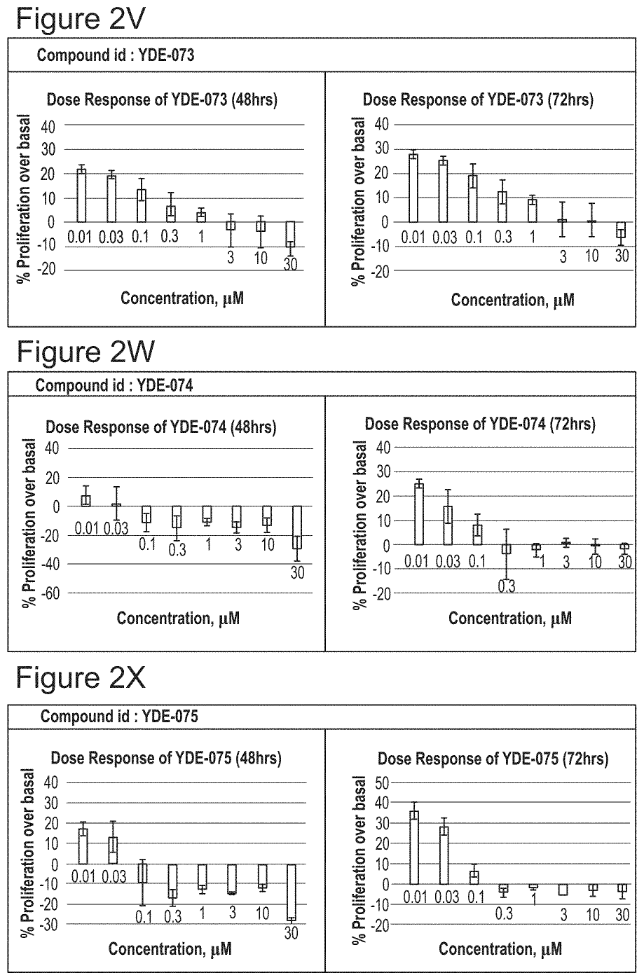

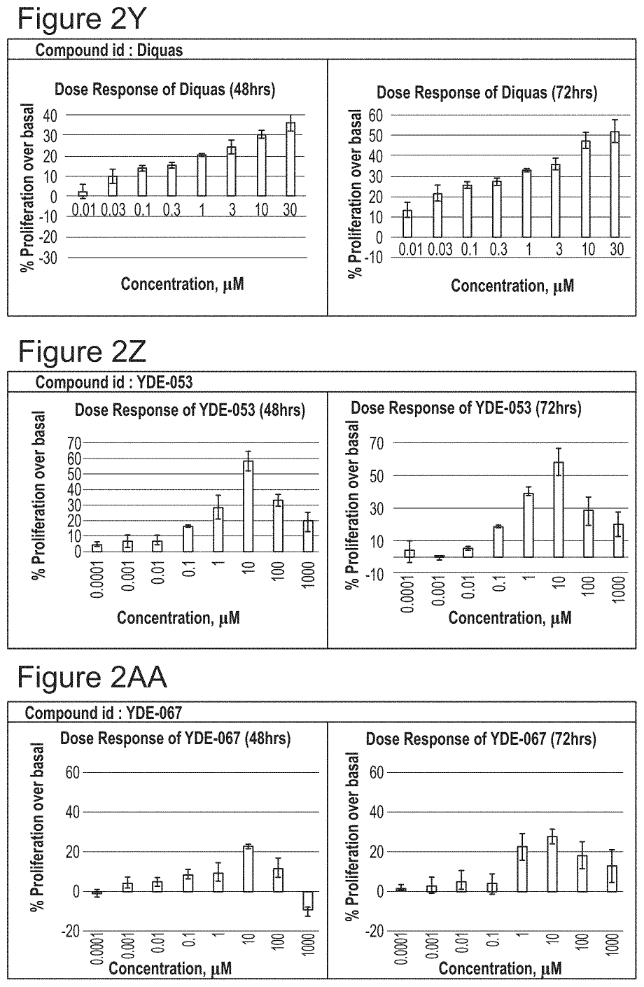

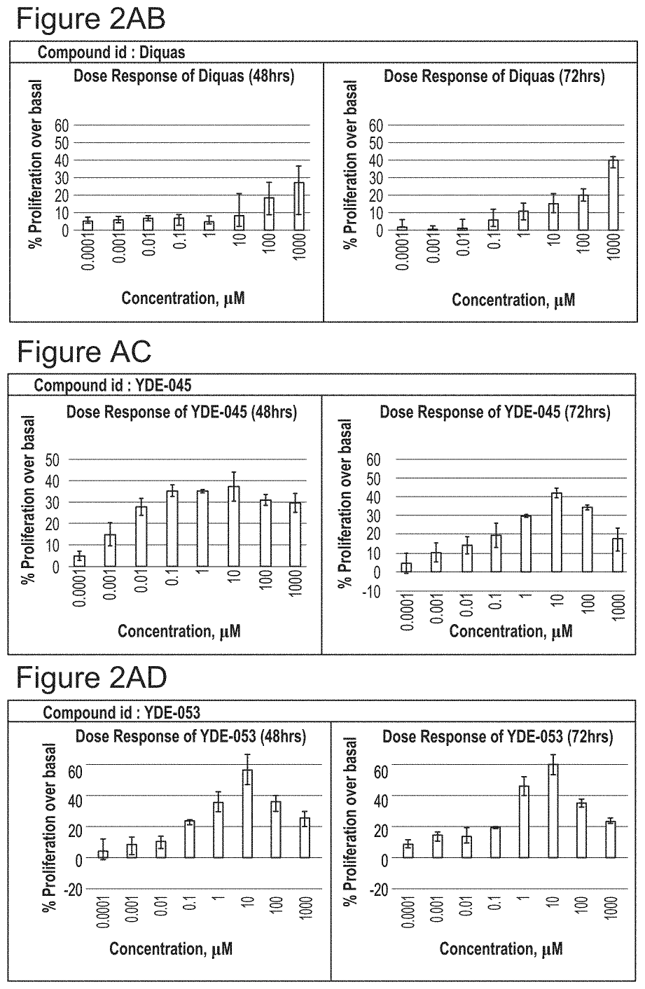

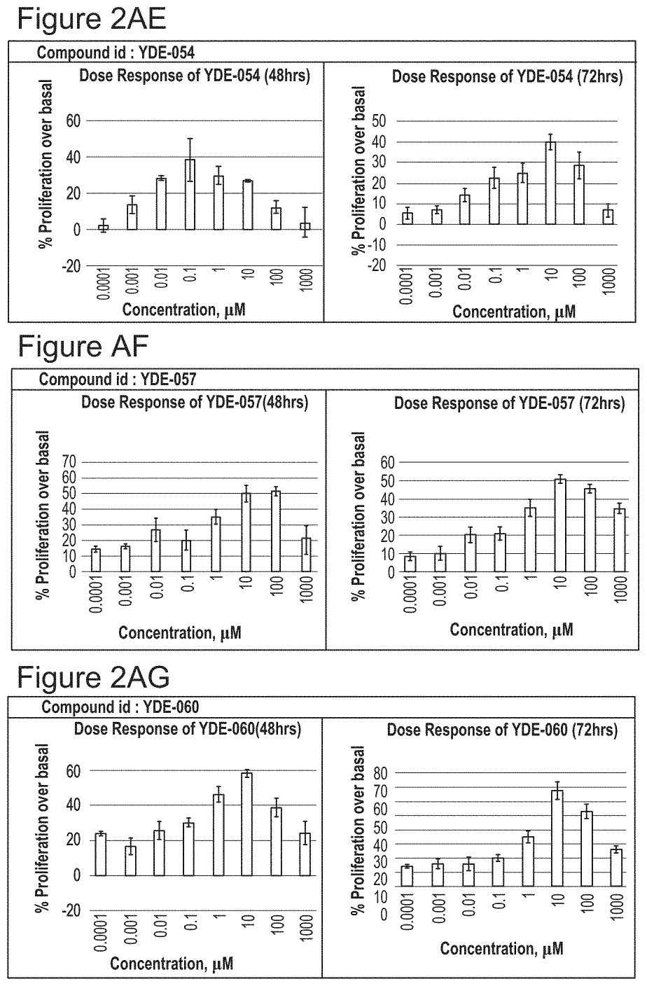

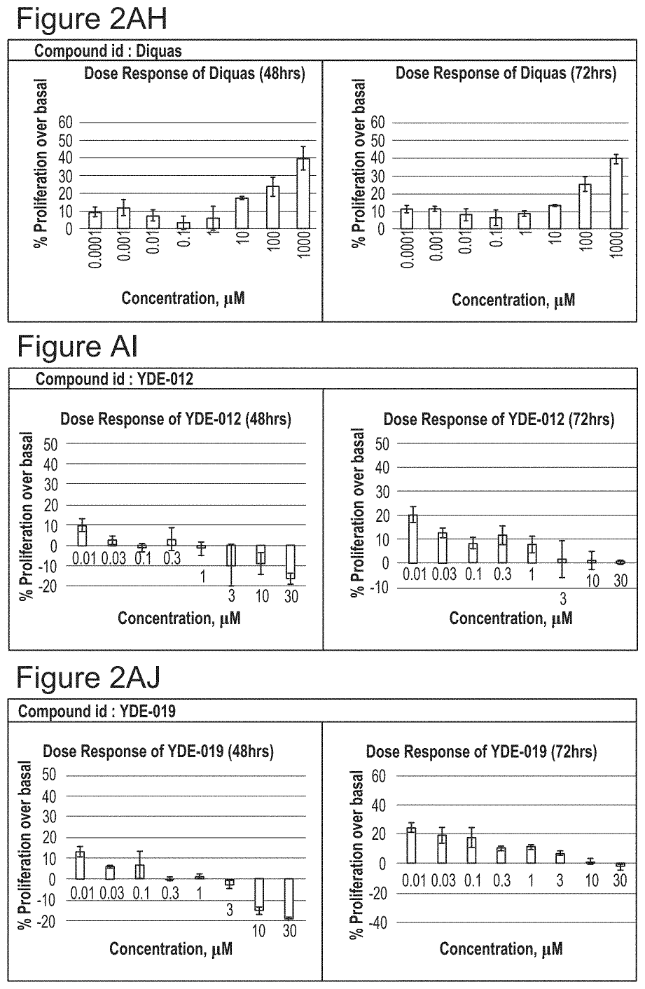

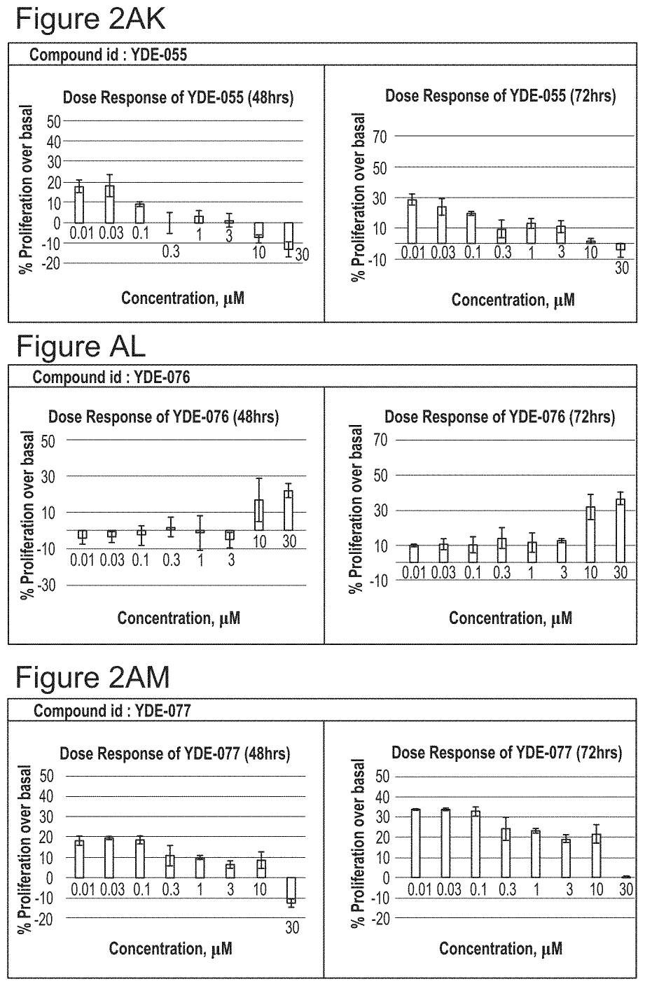

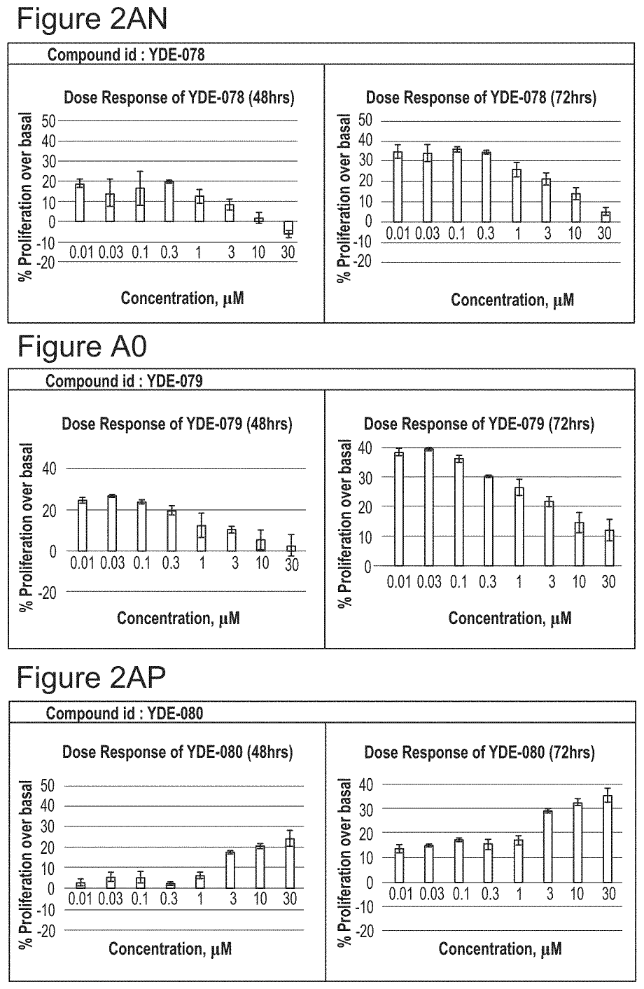

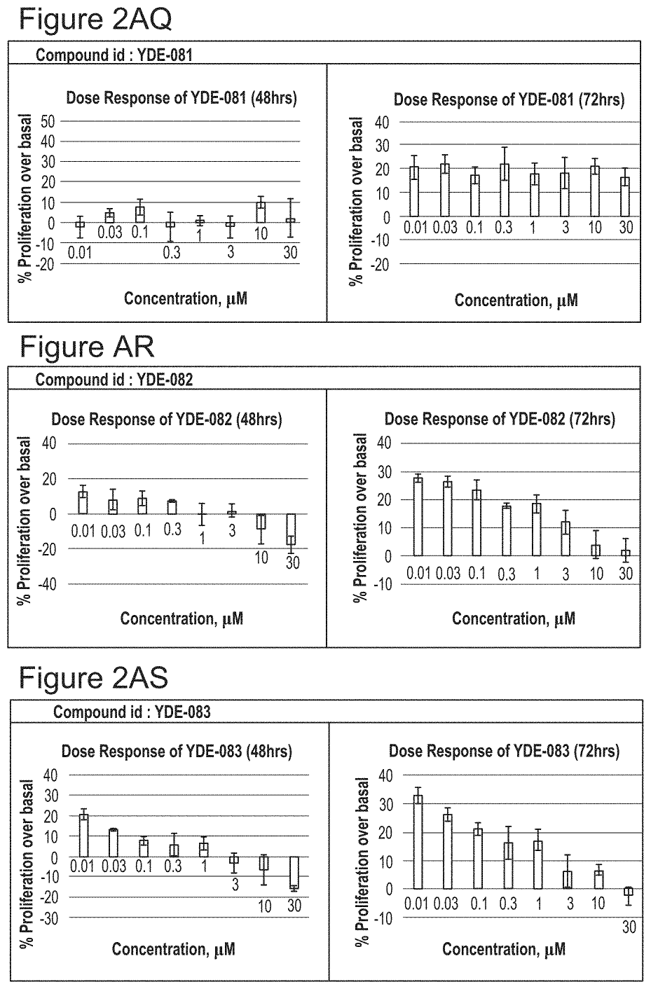

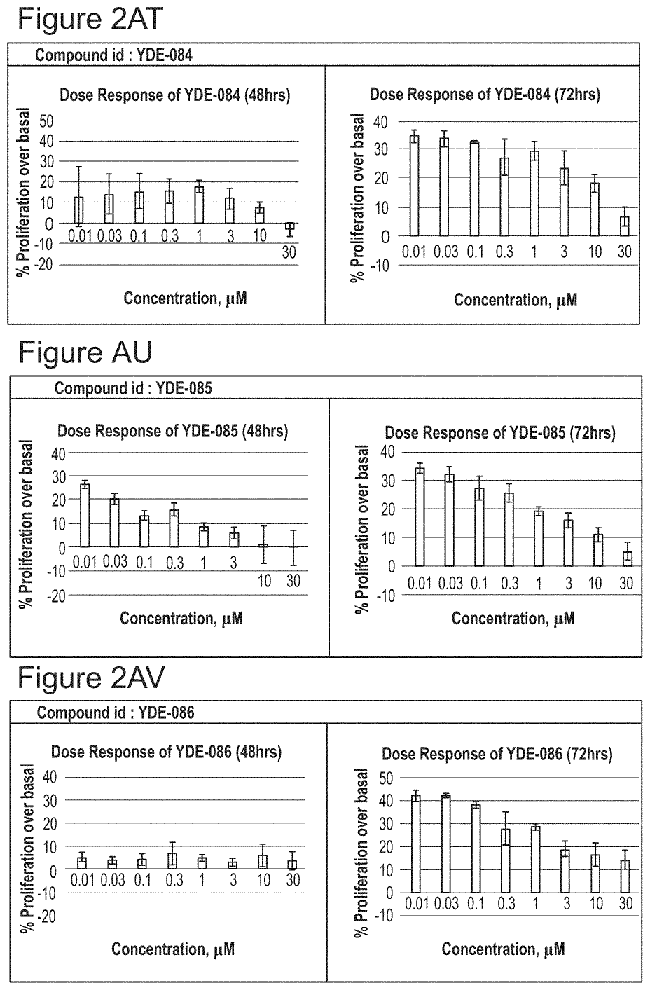

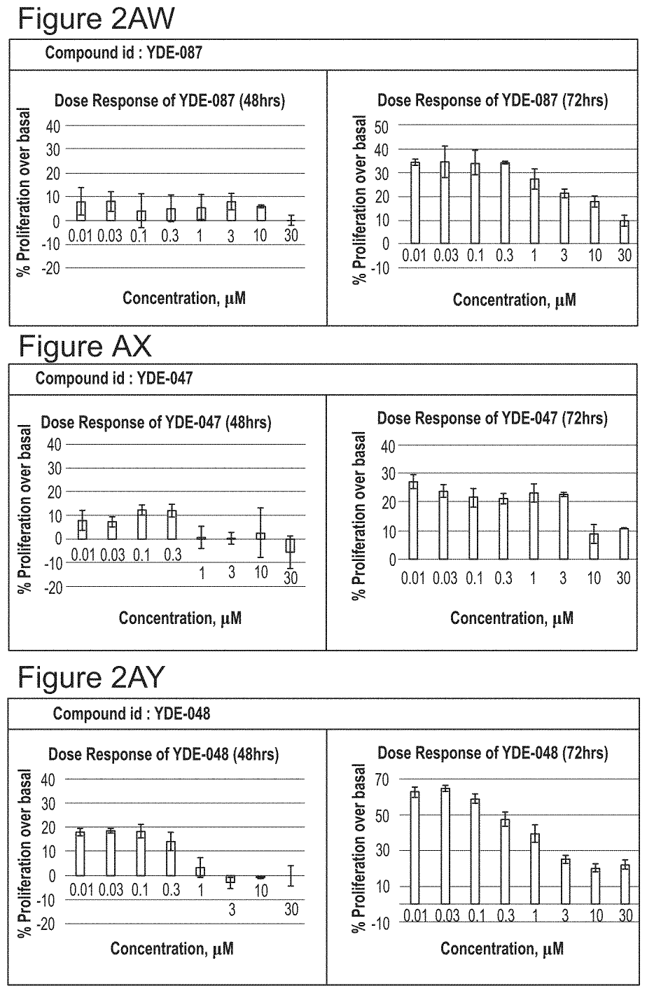

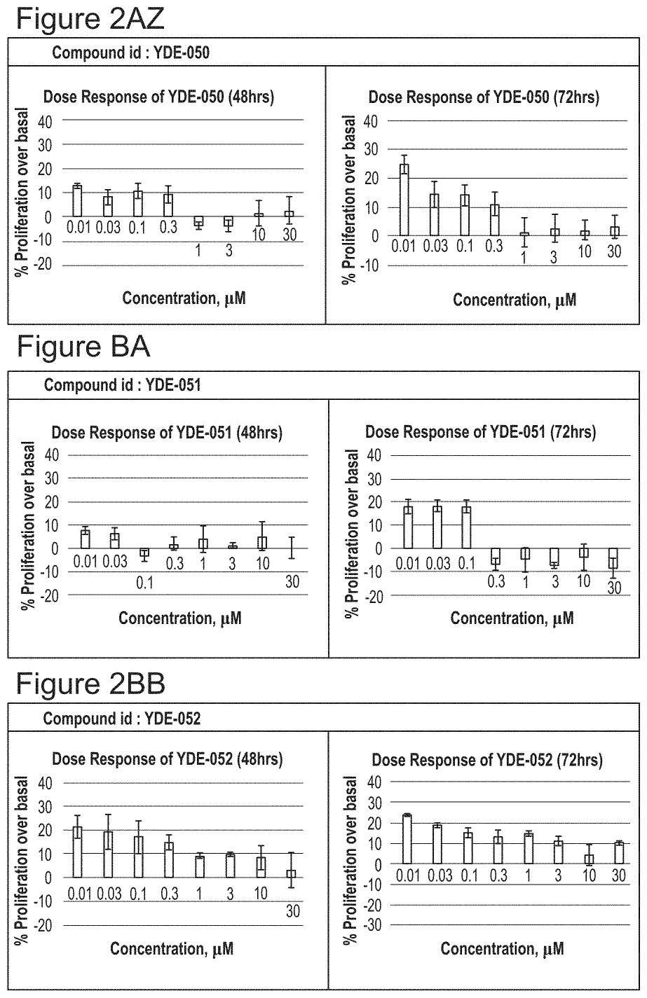

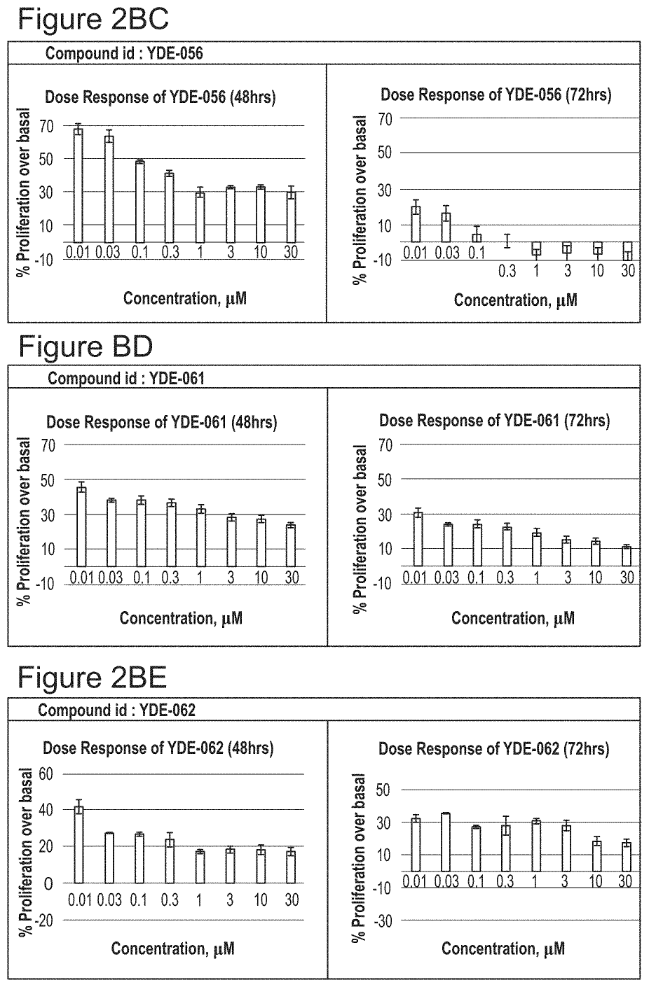

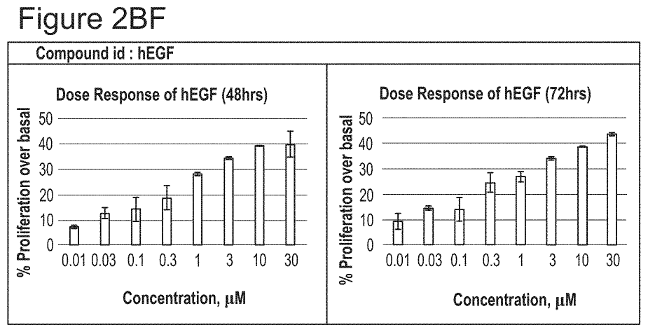

The proliferation effect of YDE-038 was assessed using human primary corneal epithelial cells. Cells were incubated with YDE-038 for 48 hrs and 72 hrs. At concentrations 30, 10, 3 and 1 μM, no significant proliferation of cells was observed (<20% over basal). This effect was observed when the cells were incubated for 48 hrs or 72 hrs. However, at lower concentrations (0.03 & 0.01 μM) an increase in cell proliferation (35-40%) in 72 hrs incubation was observed. Whereas after 48 hrs incubation there was no proliferation observed, even at lower concentrations (FIG. 2A).

The proliferation effect of YDE-044 was assessed using human primary corneal epithelial cells. Cells were incubated with YDE-044 for 48 hrs and 72 hrs. At concentrations 30 μM, no significant proliferation of cells (<20% over basal) was observed. At low concentrations of YDE-044, there was proliferation of cells (20-30%). Maximum 30% proliferation was observed at the lowest concentration (0.01 μM) (FIG. 2B).

The proliferation effect of YDE-045 was assessed using human primary corneal epithelial cells. Cells were incubated with YDE-045 for 48 hrs and 72 hrs. At concentrations 30, 10, 3 and 1 μM, no significant proliferation of cells (<20% over basal) was observed. This effect was observed when the cells were incubated with YDE-045 for 48 hrs or 72 hrs. However, at lower concentrations an increase in cell proliferation (35-50%) was observed (FIG. 2C).

The proliferation effect of YDE-049 was assessed using human primary corneal epithelial cells. Cells were incubated with YDE-049 for 48 hrs and 72 hrs. There was no significant proliferation of cells when YDE-049 was incubated at 48 hrs incubation. At 30, 10 and 3 μM the peptide showed toxic effect (˜25-40%). However, at 72 hrs incubation, at concentrations 30, 10, 3 and 1 μM, no significant proliferation of cells was observed (<20% over basal). At lower concentrations, an increase in cell proliferation (25-40%) was observed when YDE-049 was incubated with primary corneal epithelial cells for 72 hrs (FIG. 2D).

The proliferation effect of YDE-053 was assessed using human primary corneal epithelial cells. Cells were incubated with YDE-053 for 48 hrs and 72 hrs. At concentrations 30, 10 and 3 μM, no significant proliferation of cells (<20% over basal) was observed. This effect was observed when the cells were incubated for 48 hrs or 72 hrs. However, at lower concentrations an increase in cell proliferation (40-60%) was observed (FIG. 2E).

The proliferation effect of YDE-054 was assessed using human primary corneal epithelial cells. Cells were incubated with YDE-054 for 48 hrs and 72 hrs. No significant proliferation at 48 hrs incubation was observed. At concentrations 30, 10, 3 and 1 μM, no significant proliferation of cells (<20% over basal) was observed. This effect was observed when the cells were incubated for 72 hrs. However, at lower concentrations an increase in cell proliferation (25-40%) at 72 hrs incubation was observed (FIG. 2F).

The proliferation effect of YDE-058 was assessed using human primary corneal epithelial cells. Cells were incubated with YDE-058 for 48 hrs and 72 hrs. No significant proliferation at 48 hrs incubation was observed. At concentrations 30 and 10 μM, no significant proliferation of cells (<20% over basal) was observed. This effect was observed when the cells were incubated for 72 hrs. However, at lower concentrations moderate increase in cell proliferation (˜25%) at 72 hrs incubation was observed (FIG. 2G).

The proliferation effect of YDE-059 was assessed using human primary corneal epithelial cells. Cells were incubated with YDE-059 for 48 hrs and 72 hrs. At concentrations 30 μM, no significant proliferation of cells (<20% over basal) was observed. Dose dependent proliferation in tested concentrations in 72 hrs incubation with high proliferation at lower dilutions and lower proliferation at high dilutions of peptides was observed. Maximum 40% proliferation resulted in the lowest concentration 0.01 μM (FIG. 2H).

The proliferation effect of YDE-063 was assessed using human primary corneal epithelial cells. Cells were incubated with YDE-063 for 48 hrs and 72 hrs. No significant proliferation of cells in 48 hrs incubation was observed. However, at lower concentrations increase in cell proliferation (˜30%) at 72 hrs incubation was observed (FIG. 2I).

The proliferation effect of YDE-064 was assessed using human primary corneal epithelial cells. Cells were incubated with YDE-064 for 48 hrs and 72 hrs. No significant proliferation of cells in 48 hrs. incubation was observed. However, at lower concentrations (0.03 & 0.01 μM) an increase in cell proliferation (˜35%) at 72 hrs incubation time point was observed (FIG. 2J).

The proliferation effect of YDE-065 was assessed using human primary corneal epithelial cells. Cells were incubated with YDE-065 for 48 hrs and 72 hrs. At concentrations 30, 10, 3 and 1 μM, no significant proliferation of cells (<20% over basal) was observed. This effect was observed when the cells were incubated for 48 hrs or 72 hrs. However, at lower concentrations an increase in cell proliferation (˜35%) at 72 hrs incubation was observed (FIG. 2K).

The proliferation effect of YDE-066 was assessed using human primary corneal epithelial cells. Cells were incubated with YDE-066 for 48 hrs and 72 hrs. There was no significant increase in cell proliferation when the peptide was incubated with cells for 48 hrs or 72 hrs (FIG. 2L).

The proliferation effect of YDE-067 was assessed using human primary corneal epithelial cells. Cells were incubated with YDE-067 for 48 hrs and 72 hrs. At concentrations 30, 10, 3 and 1 μM, no significant proliferation of cells (<20% over basal) was observed. This effect was observed when the cells were incubated for 48 hrs or 72 hrs. However, at lower concentrations (0.03 & 0.01 μM), an increase in cell proliferation (35%) in 72 hrs incubation was observed. Whereas 48 hrs incubation no significant proliferation was observed even at lower concentrations (FIG. 2M).

The proliferation effect of YDE-045 was assessed using human primary corneal epithelial cells. Cells were incubated with YDE-045 for 48 hrs and 72 hrs. At concentrations 30, 10, μM, no significant proliferation of cells (<20% over basal) was observed. This effect was observed when the cells were incubated for 48 hrs or 72 hrs. However, at lower concentrations (0.1, 0.03 & 0.01 μM) an increase in cell proliferation (35-40%) in 72 hrs incubation was observed (FIG. 2N).

The proliferation effect of YDE-049 was assessed using human primary corneal epithelial cells. Cells were incubated with YDE-049 for 48 hrs and 72 hrs. At all tested concentrations, no significant proliferation of cells (<20% over basal) at 48 hrs was observed. Proliferation at 0.03 & 0.01 concentrations at 72 hrs incubation was observed. Maximum 30% proliferation was observed at the lowest concentration (0.01 μM) (FIG. 2O).

The proliferation effect of YDE-053 was assessed using human primary corneal epithelial cells. Cells were incubated with YDE-053 for 48 hrs and 72 hrs. At concentrations 30, 10 and 3 μM, no significant proliferation of cells (<20% over basal) was observed. This effect was observed when the cells were incubated for 48 hrs or 72 hrs. However, at lower concentrations an increase in cell proliferation (40-60%) was observed (FIG. 2P).

The proliferation effect of YDE-057 was assessed using human primary corneal epithelial cells. Cells were incubated with YDE-057 for 48 hrs and 72 hrs. Significant proliferation at 48 hrs incubation with 0.1, 0.03 & 0.01 μM concentrations was observed. At concentrations 30, 10, 3 and 1 μM, no significant proliferation of cells (<20% over basal) observed in 48 hrs incubation was observed. However, at lower concentrations an increase in cell proliferation (40-50%) at 72 hrs incubation was observed (FIG. 2Q).

The proliferation effect of YDE-060 was assessed using human primary corneal epithelial cells. Cells were incubated with YDE-060 for 48 hrs and 72 hrs. At concentrations 30, 10, 3, 1 and 0.3 μM, significant proliferation of cells (<20% over basal) was observed. This effect was observed when the cells were incubated for 72 hrs. However, at lower concentrations an increase in cell proliferation (˜40-60%) at 72 hrs incubation was observed (FIG. 2R).

The proliferation effect of YDE-065 was assessed using human primary corneal epithelial cells. Cells were incubated with YDE-065 for 48 hrs and 72 hrs. No significant proliferation of cells (<20% over basal) in 48 hrs incubation was observed. Dose dependent proliferation in tested concentrations in 72 hrs incubation with high proliferation at lower dilutions was observed. Maximum 40% proliferation resulted in the lowest concentration 0.01 μM (FIG. 2S).

The proliferation effect of YDE-067 was assessed using human primary corneal epithelial cells. Cells were incubated with YDE-067 for 48 hrs and 72 hrs. No significant proliferation of cells in 48 hrs Incubation. However, at lower concentrations an increase in cell proliferation (˜30%) at 72 hrs incubation was observed (FIG. 2T)

The proliferation effect of YDE-072 was assessed using human primary corneal epithelial cells. Cells were incubated with YDE-072 for 48 hrs and 72 hrs. No significant proliferation of cells in 48 hrs. Incubation except 0.03 & 0.01 μM concentrations. However, at lower concentrations (0.03 & 0.01 μM) an increase in cell proliferation (˜30%) at 72 hrs incubation time point was observed (FIG. 2U).

The proliferation effect of YDE-073 was assessed using human primary corneal epithelial cells. Cells were incubated with YDE-073 for 48 hrs and 72 hrs. No significant proliferation of cells (<20% over basal) was observed. This effect was observed when the cells were incubated for 48 hrs. However, at lower concentrations an increase in cell proliferation (˜30%) at 72 hrs incubation was observed.

The proliferation effect of YDE-074 was assessed using human primary corneal epithelial cells. Cells were incubated with YDE-074 for 48 hrs and 72 hrs. There was no significant increase in cell proliferation when the peptide was incubated with cells for 48 hrs or 72 hrs (FIG. 2W).

The proliferation effect of YDE-075 was assessed using human primary corneal epithelial cells. Cells were incubated with YDE-075 for 48 hrs and 72 hrs. There was no significant increase in cell proliferation when the peptide was incubated with cells for 48 hrs. However, at lower concentrations (0.03 & 0.01 μM) an increase in cell proliferation (˜30%) at 72 hrs incubation time point was observed (FIG. 2X).

The proliferation effect of Diquas was assessed using human primary corneal epithelial cells. Cells were incubated with Diquas for 48 hrs and 72 hrs. Dose dependent increase in cell proliferation when the peptide was incubated with cells for 48 hrs or 72 hrs was observed (FIG. 2Y).

The proliferation effect of YDE-053 was assessed using human primary corneal epithelial cells. Cells were incubated with YDE-053 for 48 hrs and 72 hrs. An increased proliferation of cells up to 10M (>20% over basal) was observed. This effect was observed when the cells were incubated for 48 hrs or 72 hrs. However, at lower concentrations no significant proliferation (i.e from 0.1 nM to 0.0001 nM) was observed (FIG. 2Z).

The proliferation effect of YDE-067 was assessed using human primary corneal epithelial cells. Cells were incubated with YDE-067 for 48 hrs and 72 hrs. No significant proliferation of cells with 48 hrs incubation of compound was observed. Whereas in 72 hrs an increased proliferation up to 10 nM was observed. However, at lower concentrations a decrease in cell proliferation (0.1 nM to 0.0001 nM) was observed (FIG. 2AA).

The proliferation effect of Diquas was assessed using human primary corneal epithelial cells. Cells were incubated with Diquas for 48 hrs and 72 hrs. Dose dependent increase in cell proliferation when the peptide was incubated with cells for 48 hrs or 72 hrs was observed. There is no significant proliferation observed from 1 nM to 0.0001 nM (FIG. 2AB).

The proliferation effect of YDE-045 was assessed using human primary corneal epithelial cells. Cells were incubated with YDE-045 for 48 hrs and 72 hrs. An increased proliferation of cells up to 10 nM (˜35% over basal) was observed. This effect was observed in 48 hrs incubation. In 72 hrs incubation concentration dependent increase proliferation observed up to 10 nM. However, at lower concentrations no significant proliferation was observed (i.e. 0.001 nM & 0.0001 nM) (FIG. 2AC).

The proliferation effect of YDE-053 was assessed using human primary corneal epithelial cells. Cells were incubated with YDE-053 for 48 hrs and 72 hrs. An increased proliferation of cells up to 10 nM (<20% over basal) was observed. This effect was observed when the cells were incubated for 48 hrs or 72 hrs. However, at lower concentrations no significant proliferation was observed (i.e. 0.1 nM to 0.0001 nM) (FIG. 2AD).

The proliferation effect of YDE-054 was assessed using human primary corneal epithelial cells. Cells were incubated with YDE-054 for 48 hrs and 72 hrs. An increased proliferation of cells up to 0.1 nM (˜30% over basal) was observed. This effect was observed when the cells were incubated for 48 hrs, whereas at 72 hrs increased proliferation up to 10 nM with 40% proliferation. At higher concentration (1000 nM) there was no significant proliferation was observed (FIG. 2AE).

The proliferation effect of YDE-057 was assessed using human primary corneal epithelial cells. Cells were incubated with YDE-057 for 48 hrs and 72 hrs. An increased proliferation of cells up to 10 nM was observed. This effect was observed when the cells were incubated for 48 hrs or 72 hrs. However, at lower concentrations no significant proliferation was observed (i.e. 0.1 nM to 0.0001 nM) (FIG. 2AF).

The proliferation effect of YDE-060 was assessed using human primary corneal epithelial cells. Cells were incubated with YDE-060 for 48 hrs and 72 hrs. Significant proliferation of cells in 48 hrs as well as 72 hrs was observed. Increased proliferation up to 10 nM with ˜60% proliferation. However, at lower concentrations no significant cell proliferation was observed (0.01 nM to 0.0001 nM) (FIG. 2AG).

The proliferation effect of Diquas was assessed using human primary corneal epithelial cells. Cells were incubated with Diquas for 48 hrs and 72 hrs. Dose dependent increase in cell proliferation when the peptide was incubated with cells for 48 hrs or 72 hrs was observed. There is no significant proliferation observed from InM to 0.0001 nM (FIG. 2AH).

The proliferation effect of YDE-012 was assessed using human primary corneal epithelial cells. Cells were incubated with YDE-012 for 48 hrs and 72 hrs. All tested concentration no significant proliferation of cells was observed (<20% over basal). This effect was observed when the cells were incubated for 48 hrs or 72 hrs (FIG. 2AI).

The proliferation effect of YDE-019 was assessed using human primary corneal epithelial cells. Cells were incubated with YDE-019 for 48 hrs and 72 hrs. No significant proliferation of cells was observed (<20% over basal). This effect was observed when the cells were incubated for 48 hrs or 72 hrs. However, at lower concentrations (0.1, 0.03 & 0.01 μM) an increase in cell proliferation (˜20%) in 72 hrs incubation was observed (FIG. 2AJ).

The proliferation effect of YDE-055 was assessed using human primary corneal epithelial cells. Cells were incubated with YDE-055 for 48 hrs and 72 hrs. No significant proliferation of cells was observed (<20% over basal). This effect was observed when the cells were incubated for 48 hrs or 72 hrs. However, at lower concentrations (0.03 & 0.01 μM) an increase in cell proliferation (25-30%) in 72 hrs incubation was observed (FIG. 2AK).

The proliferation effect of YDE-076 was assessed using human primary corneal epithelial cells. Cells were incubated with YDE-076 for 48 hrs and 72 hrs. At concentrations 30, 10, μM, significant proliferation of cells was observed (>20% over basal). This effect was observed when the cells were incubated for 48 hrs or 72 hrs. Other tested concentration no significant proliferation observed (FIG. 2AL).

The proliferation effect of YDE-077 was assessed using human primary corneal epithelial cells. Cells were incubated with YDE-077 for 48 hrs and 72 hrs. No significant proliferation of cells was observed (<20% over basal). This effect was observed when the cells were incubated for 48 hrs. At 72 hrs incubation an increasing proliferation from 10 μM to 0.01 μM was observed (FIG. 2AM).

The proliferation effect of YDE-078 was assessed using human primary corneal epithelial cells. Cells were incubated with YDE-078 for 48 hrs and 72 hrs. No significant proliferation of cells was observed (<20% over basal). This effect was observed when the cells were incubated for 48 hrs. At 72 hrs incubation an increasing proliferation from 3 μM to 0.01 μM was observed (FIG. 2AN).

The proliferation effect of YDE-079 was assessed using human primary corneal epithelial cells. Cells were incubated with YDE-079 for 48 hrs and 72 hrs. No significant proliferation of cells (<20% over basal) at higher concentration was observed (30-0.3 μM). This effect was observed when the cells were incubated for 48 hrs. At 72 hrs incubation an increasing significant proliferation from 30 μM to 0.01 μM (˜35% cell proliferation) was observed (FIG. 2AO).

The proliferation effect of YDE-080 was assessed using human primary corneal epithelial cells. Cells were incubated with YDE-080 for 48 hrs and 72 hrs. At concentrations 30, 10 &3 μM, dependent proliferation of cells was observed (>20% over basal). This effect was observed when the cells were incubated for 48 hrs or 72 hrs. However, at lower concentrations (1.0 to 0.01 μM) no significant cell proliferation in 48 hrs as well as 72 hrs incubation was observed (FIG. 2AP).

The proliferation effect of YDE-081 was assessed using human primary corneal epithelial cells. Cells were incubated with YDE-081 for 48 hrs and 72 hrs. All tested concentrations no significant proliferation of cells was observed (<20% over basal). This effect was observed when the cells were incubated for 48 hrs or 72 hrs (FIG. 2AQ).

The proliferation effect of YDE-082 was assessed using human primary corneal epithelial cells. Cells were incubated with YDE-082 for 48 hrs and 72 hrs. All tested concentrations, no significant proliferation of cells was observed (<20% over basal). This effect was observed when the cells were incubated for 48 hrs. At 72 hrs incubation an increasing proliferation from 1 μM to 0.01 μM was observed (FIG. 2AR).

The proliferation effect of YDE-083 was assessed using human primary corneal epithelial cells. Cells were incubated with YDE-083 for 48 hrs and 72 hrs. All tested concentrations no significant proliferation of cells (<20% over basal) was observed. This effect was observed when the cells were incubated for 48 hrs. At 72 hrs incubation an increasing proliferation from 1 μM to 0.01 μM was observed (FIG. 2AS).

The proliferation effect of YDE-084 was assessed using human primary corneal epithelial cells. Cells were incubated with YDE-084 for 48 hrs and 72 hrs. All tested concentrations, no significant proliferation of cells (<20% over basal) was observed. This effect was observed when the cells were incubated for 48 hrs. At 72 hrs incubation an increasing proliferation from 10 μM to 0.01 μM was observed (FIG. 2AT).

The proliferation effect of YDE-085 was assessed using human primary corneal epithelial cells. Cells were incubated with YDE-085 for 48 hrs and 72 hrs. Tested concentrations 30 μM to 0.1 μM, no significant proliferation of cells (<20% over basal) was observed. This effect was observed when the cells were incubated for 48 hrs, whereas 0.03 & 0.01 μM slightly increased proliferation. At 72 hrs incubation an increasing proliferation from 1 μM to 0.01 μM was observed (FIG. 2AU).

The proliferation effect of YDE-086 was assessed using human primary corneal epithelial cells. Cells were incubated with YDE-086 for 48 hrs and 72 hrs. All tested concentrations, no significant proliferation of cells (<20% over basal) was observed. This effect was observed when the cells were incubated for 48 hrs. At 72 hrs incubation an increasing proliferation from 1 μM to 0.01 μM (FIG. 2AV).

The proliferation effect of YDE-087 was assessed using human primary corneal epithelial cells. Cells were incubated with YDE-087 for 48 hrs and 72 hrs. All tested concentrations, no significant proliferation of cells (<20% over basal) was observed. This effect was observed when the cells were incubated for 48 hrs. At 72 hrs incubation an increasing proliferation from 3 μM to 0.01 μM was observed (FIG. 2AW).

The proliferation effect of YDE-047 was assessed using human primary corneal epithelial cells. Cells were incubated with YDE-047 for 48 hrs and 72 hrs. No significant proliferation of cells was observed (<20% over basal). This effect was observed when the cells were incubated for 48 hrs. At lower concentrations (3 to 0.01 μM). An increase in cell proliferation (25-30%) in 72 hrs incubation was observed. (FIG. 2AX).

The proliferation effect of YDE-048 was assessed using human primary corneal epithelial cells. Cells were incubated with YDE-048 for 48 hrs and 72 hrs. All tested concentrations, no significant proliferation of cells was observed (<20% over basal). This effect was observed when the cells were incubated for 48 hrs. At 72 hrs incubation an increasing proliferation from 1 μM to 0.01 μM was observed (FIG. 2AY).

The proliferation effect of YDE-050 was assessed using human primary corneal epithelial cells. Cells were incubated with YDE-050 for 48 hrs and 72 hrs. All tested concentrations, no significant proliferation of cells was observed (<20% over basal). This effect was observed when the cells were incubated for 48 hrs or 72 hrs. However, at lower concentration 0.01 μM, an increase in cell proliferation (30%) in 72 hrs incubation was observed (FIG. 2AZ).

The proliferation effect of YDE-051 was assessed using human primary corneal epithelial cells. Cells were incubated with YDE-051 for 48 hrs and 72 hrs. All tested concentrations, no significant proliferation of cells (<20% over basal) was observed. This effect was observed when the cells were incubated for 48 hrs or 72 hrs (FIG. 2BA).

The proliferation effect of YDE-052 was assessed using human primary corneal epithelial cells. Cells were incubated with YDE-052 for 48 hrs and 72 hrs. All tested concentrations, no significant proliferation of cells (<20% over basal) was observed. This effect was observed when the cells were incubated for 48 hrs or 72 hrs (FIG. 2BB).

The proliferation effect of YDE-056 was assessed using human primary corneal epithelial cells. Cells were incubated with YDE-056 for 48 hrs and 72 hrs. At concentrations 30 to 0.01, μM, significant proliferation of cells (>20% over basal) was observed. This effect was observed when the cells were incubated for 48 hrs whereas at 72 hrs incubation no significant proliferation was observed (FIG. 2BC).

The proliferation effect of YDE-061 was assessed using human primary corneal epithelial cells. Cells were incubated with YDE-061 for 48 hrs and 72 hrs. At concentrations 30 to 0.01, μM, significant proliferation of cells (>20% over basal) was observed. This effect was observed when the cells were incubated for 48 hrs whereas at 72 hrs incubation no significant proliferation was observed (FIG. 2BD).

The proliferation effect of YDE-062 was assessed using human primary corneal epithelial cells. Cells were incubated with YDE-062 for 48 hrs and 72 hrs. All tested concentrations, no significant proliferation of cells (<20% over basal) was observed. This effect was observed when the cells were incubated for 48 hrs where as 72 hrs showed slightly increase in proliferation (FIG. 2BE).

Human epidermal growth factor (hEGF) as used as reference compound to assess the cell proliferation assays. Concentration dependent proliferation of primary corneal epithelial cells was observed. A maximum of ˜40% proliferation at highest tested concentration of 30 μM at 48 hrs incubation was observed (FIG. 2BF).

### Example 4: Assessment of the Effect of YDE053, YDE060, or YDE065 on Soluble Proteins Release in Poly I:C Stimulated Primary Human Corneal Epithelial Cells

### Study Objective

To determine the effect of compound by assessing the panel of soluble proteins release in poly I:C stimulated primary human corneal epithelial cells by Multiplex.

### Study Plan

Test System: Primary Human Corneal Epithelial cells
Assay Format: 96-well plate format
Method of Detection: Enzyme-linked immunosorbent assay
Assay Controls: Hinokitiol

### Experimental Design

Assay protocol as described below was used to determine the significant effect of test compounds. The data was statistically analyzed by one-way ANOVA with a Bonferroni post test comparing all the columns. The significance was represented by the p value.

### Results & Conclusion

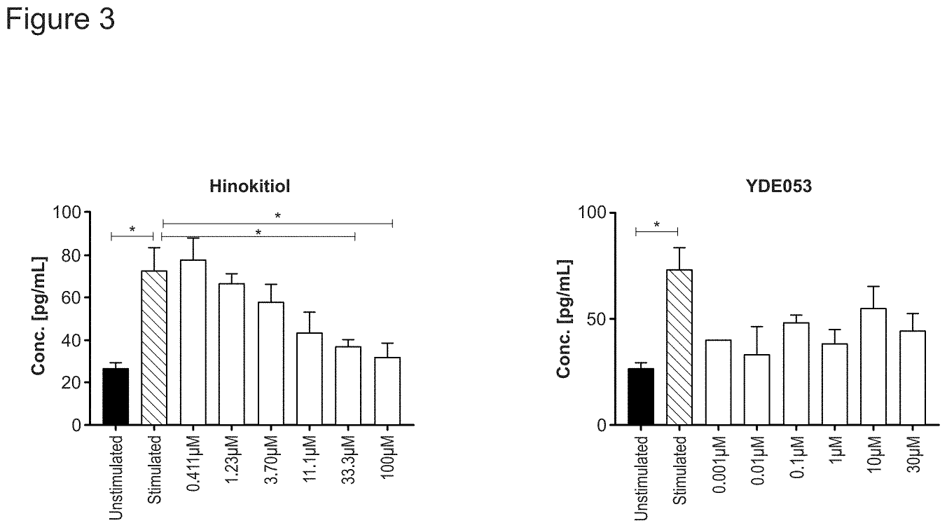

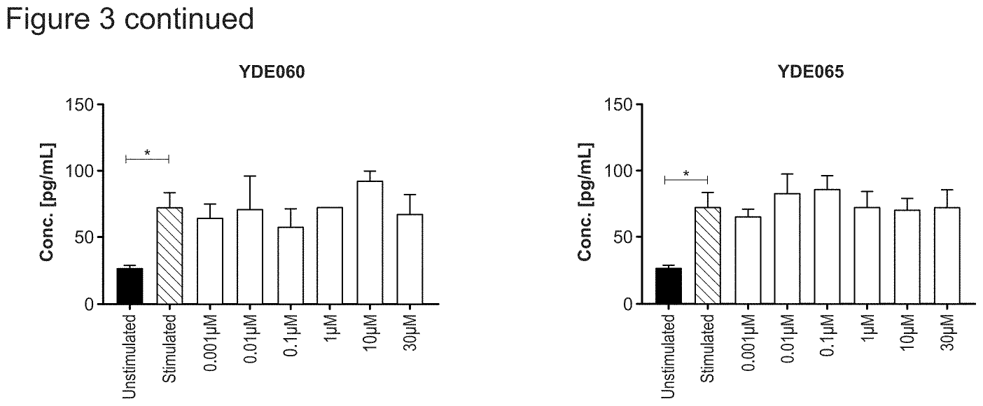

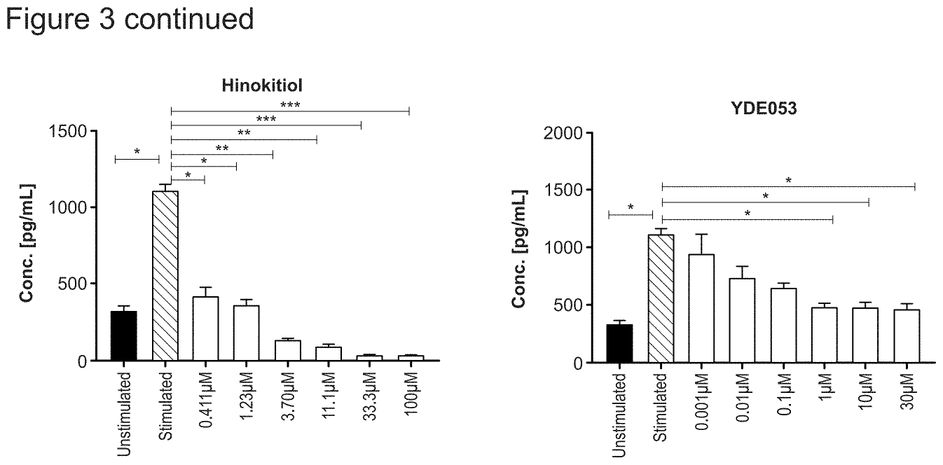

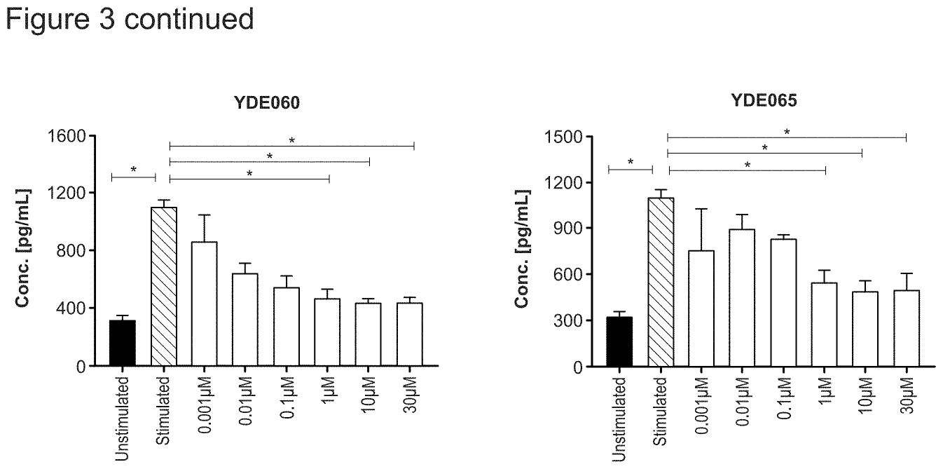

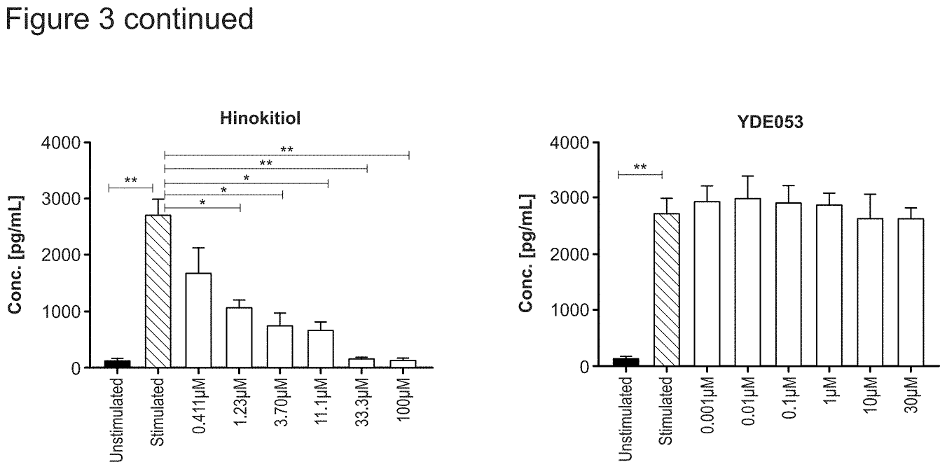

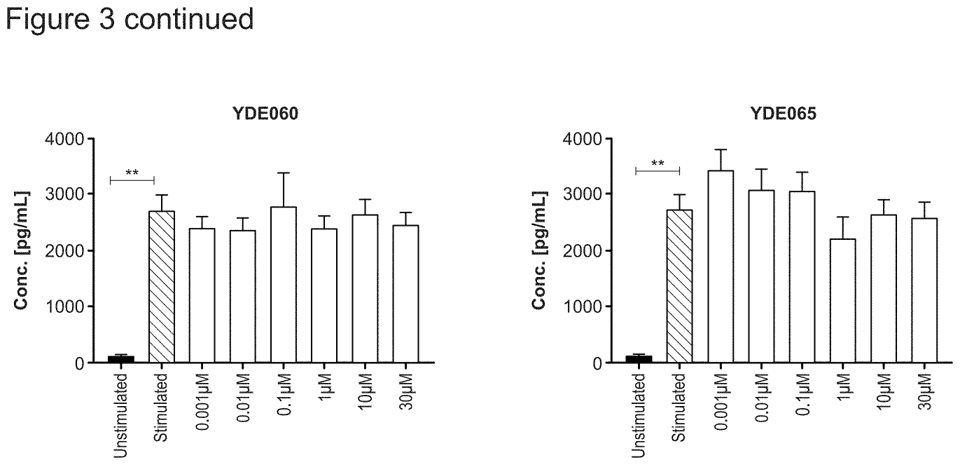

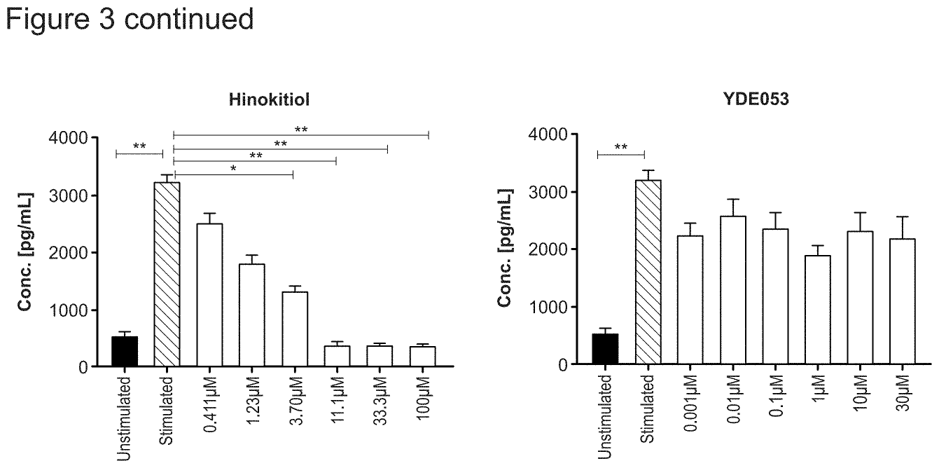

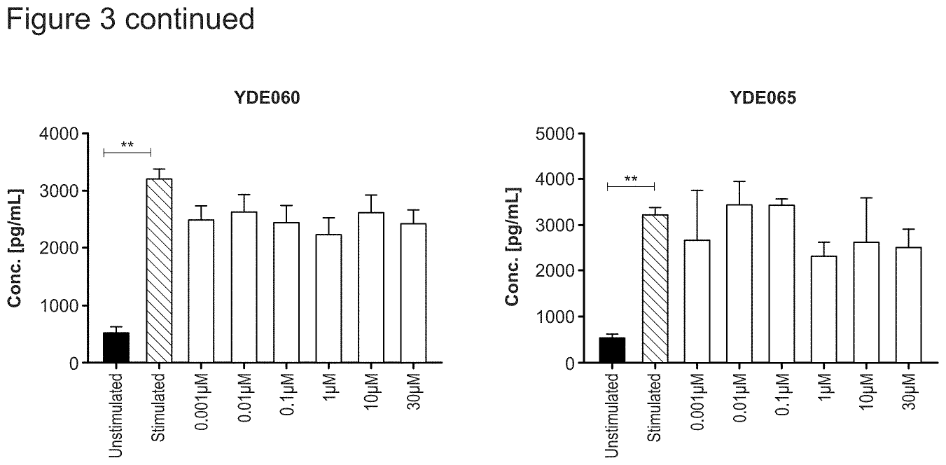

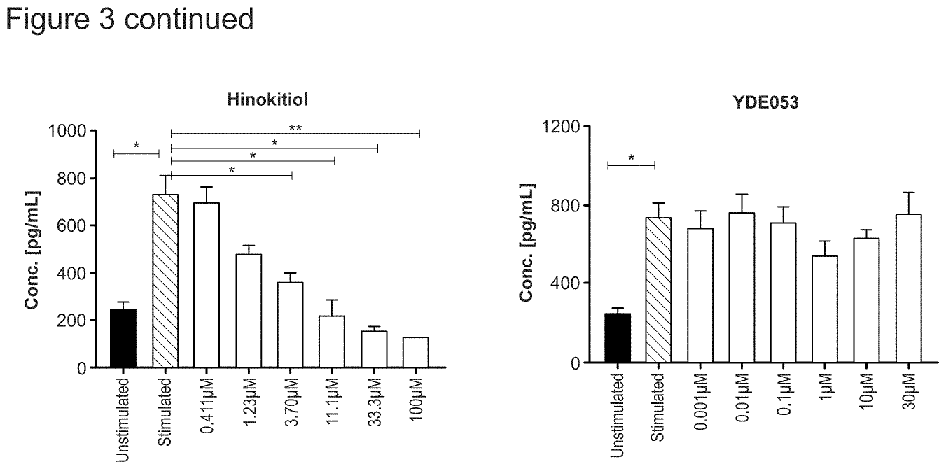

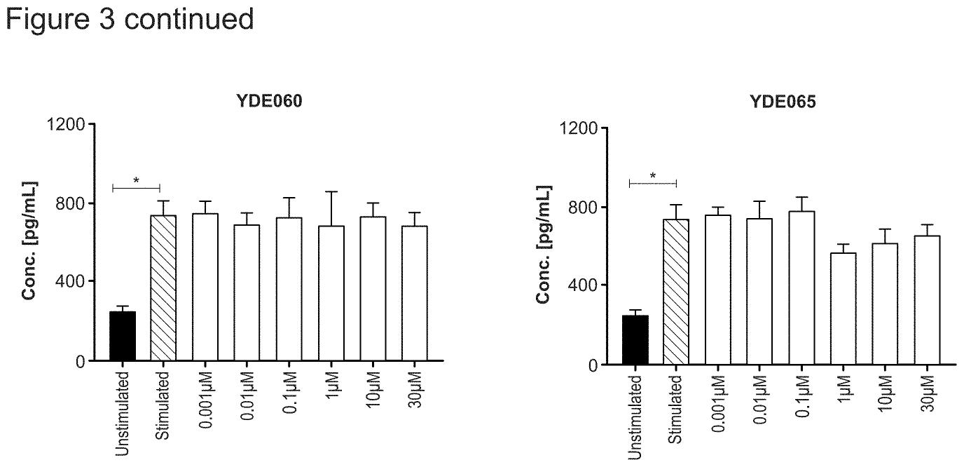

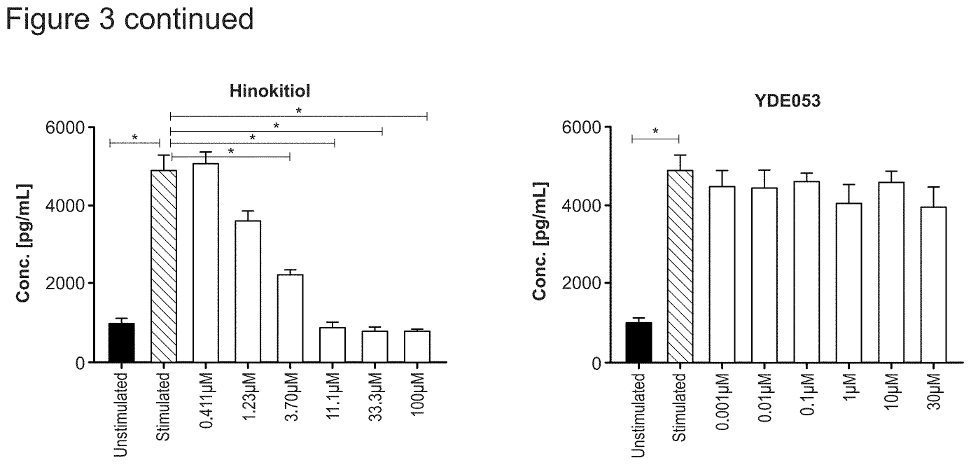

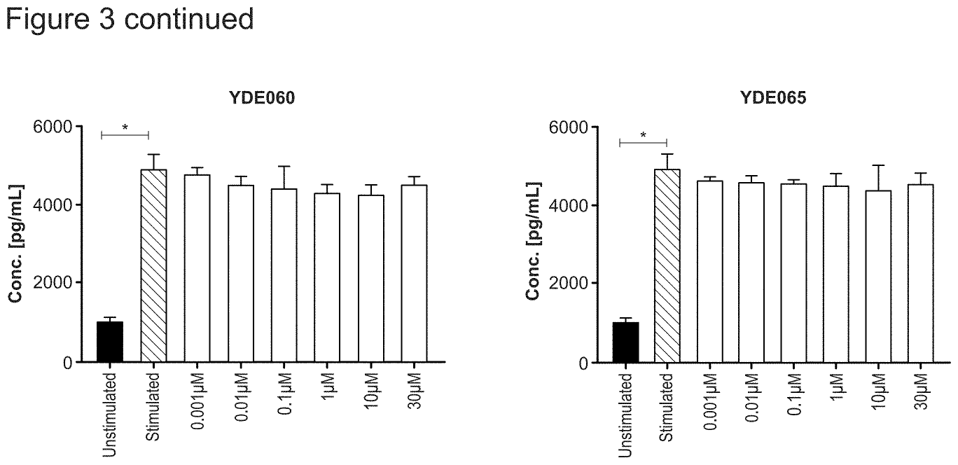

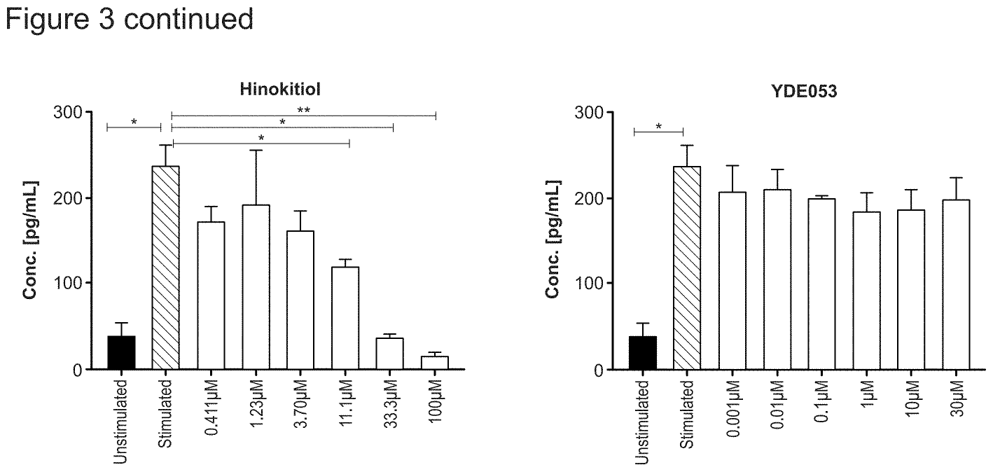

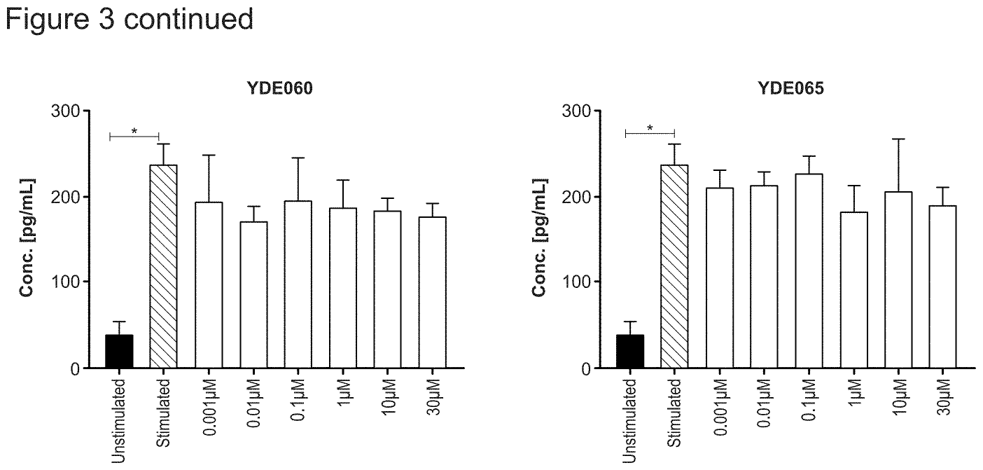

Results are shown in FIG. 3. IL-1beta, IL-6, IL-8, MIP-1alpha, MIP-1beta, RANTES, and TNF-alpha cytokine release was observed in human corneal epithelial cells. Stimulation with Poly I:C resulted in a significantly increase of IL-1beta, IL-6, IL-8, MIP-1alpha, MIP-1beta, RANTES, and TNF-alpha cytokine levels.

Reference compound, Hinokitiol, was found to significantly attenuate the Poly I:C stimulated cytokines release (IL-1beta, IL-6, IL-8, MIP-1alpha, MIP-1beta, RANTES, and TNF-alpha) in a dose dependent manner.

Test compounds YDE053, YDE060 and YDE065 were found to attenuate Poly I:C induced IL-6 cytokine release in a dose dependent manner. This was corroborated by the significant attenuation of IL-6 cytokine release at the tested concentrations that included 30 μM, 10 μM, and 1 μM respectively.

Effect of YDE053 on IL-6: Stimulated (MFI Mean±SD: 14801±407) vs Stimulated+30 μM Hinokitiol (MFI Mean±SD: 6759±500); p<0.05, Stimulated vs Stimulated+10 μM Hinokitiol (MFI Mean±SD: 7011±471) p<0.05, and Stimulated vs. Stimulated+1 μM Hinokitiol (MFI Mean±SD: 7004±376) p<0.05.

### Example 5: Determine the Effect of YDE053 Peptide on NF-kB (p65) Transcription Activity in Poly I:C Stimulated Primary Human Corneal Epithelial Cells

### Study Objective

To determine the effect of YDE053 on NF-kB (p65) transcription activity in poly I:C stimulated primary human corneal epithelial cells.

Study Plan
Test System: Primary Human Corneal Epithelial cells
Assay Format: 96-well plate format
Method of Detection: Enzyme-linked immunosorbent assay
Assay Controls: Hinokitiol

### Experimental Design

Assay protocol as shown below was used to determine the significant effect of test compounds. The data was statistically analyzed by one-way ANOVA with a Bonferroni post test comparing all the columns. The significance was represented by the p value.

### Results & Conclusion

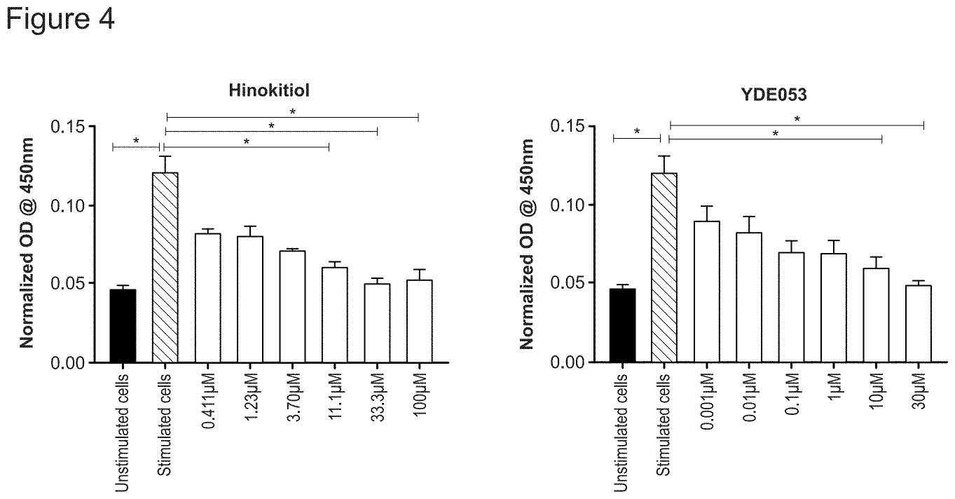

Results are shown in FIG. 4. Poly I:C stimulation on primary human corneal epithelial cells was found to result in the phosphorylation of (p65) NF-kB (Unstimulated cells: OD Mean±SD: 0.047±0.002 vs Stimulated: OD Mean±SD: 0.121±0.01; p<0.05).

Reference compound, Hinokitiol was found to significantly attenuate the phosphorylation of (p65) NF-kB in Poly I:C stimulated cells in a dose dependent manner. The effect of YDE053 was found to significantly attenuate the phosphorylation of (p65) NF-kB in Poly I: C induced cells. This was corroborated by stimulated cells (OD Mean±SD: 0.121±0.01) vs cells treated with 30 μM YDE053 (OD Mean±SD: 0.049±0.002); p<0.05, Stimulated cells (OD Mean±SD: 0.121±0.01) vs Cells treated with 10 μM YDE053 (OD Mean±SD: 0.06±0.006) p<0.05. Test compound YDE053 reduced (p65) NF-kB transcription activity in a dose dependent manner at the tested concentrations.

### Example 6: Determine the Effect of Compounds on Soluble IL6 Cytokine Release in Poly I:C Stimulated Primary Human Corneal Epithelial Cells

### Study Objective

To determine the effect of compound by assessing the soluble IL-6 cytokine release in poly I:C stimulated primary human corneal epithelial cells by ELISA.

### Study Plan

Test System: Primary Human Corneal Epithelial cells
Assay Format: 96-well plate format
Method of Detection: Enzyme-linked immunosorbent assay
Assay Controls: Hinokitiol

### Experimental Design

Assay protocol as shown below was used to determine the significant effect of test compounds. The data was statistically analyzed by one-way ANOVA with a Bonferroni post test comparing all the columns. The significance was represented by the p value.

### Results & Conclusion

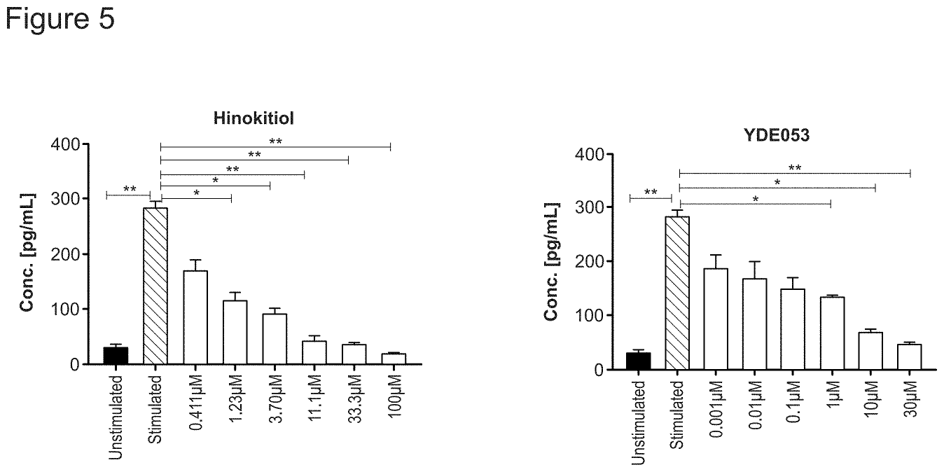

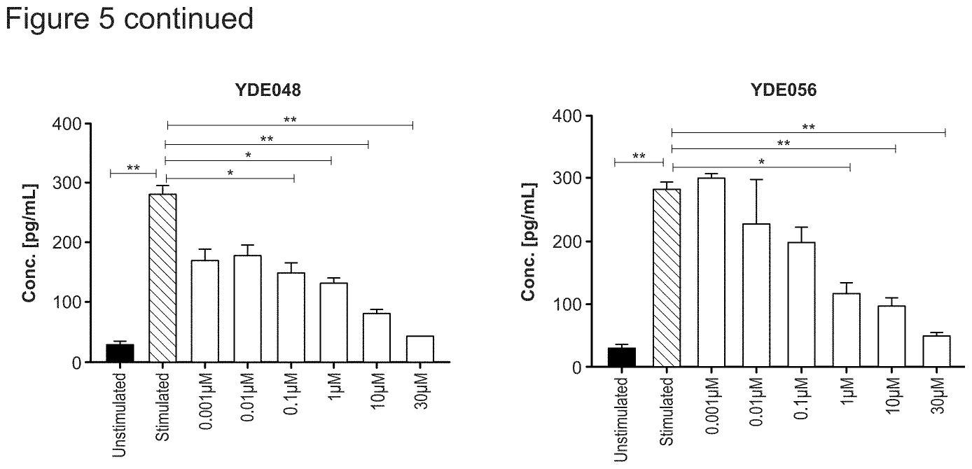

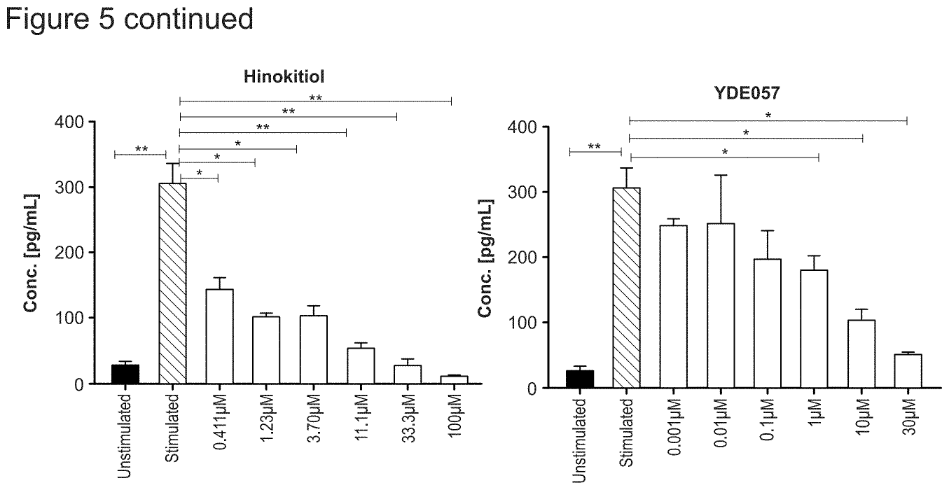

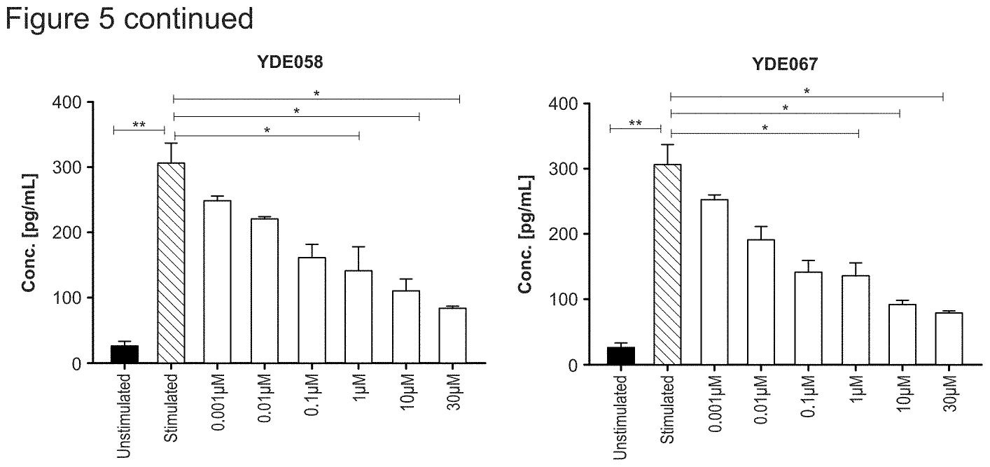

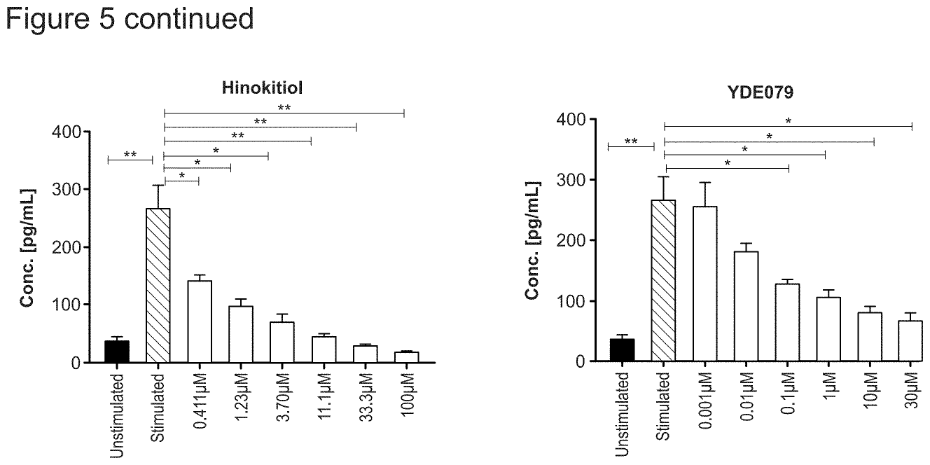

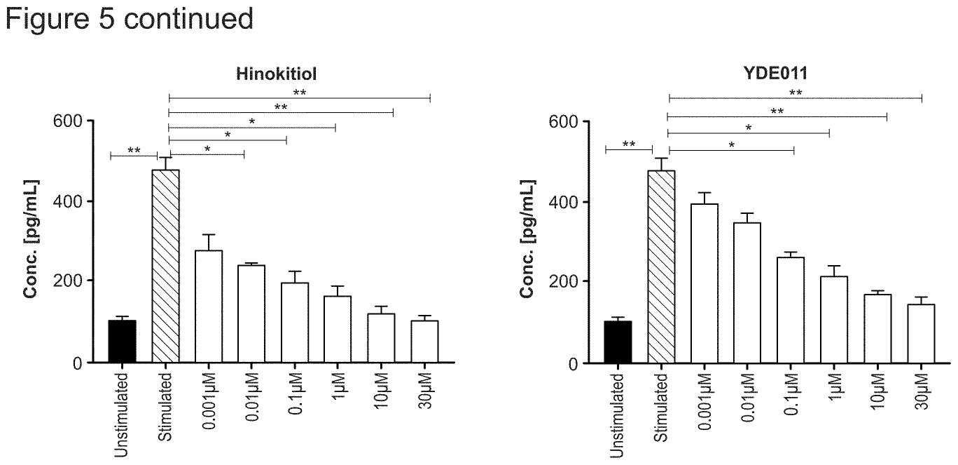

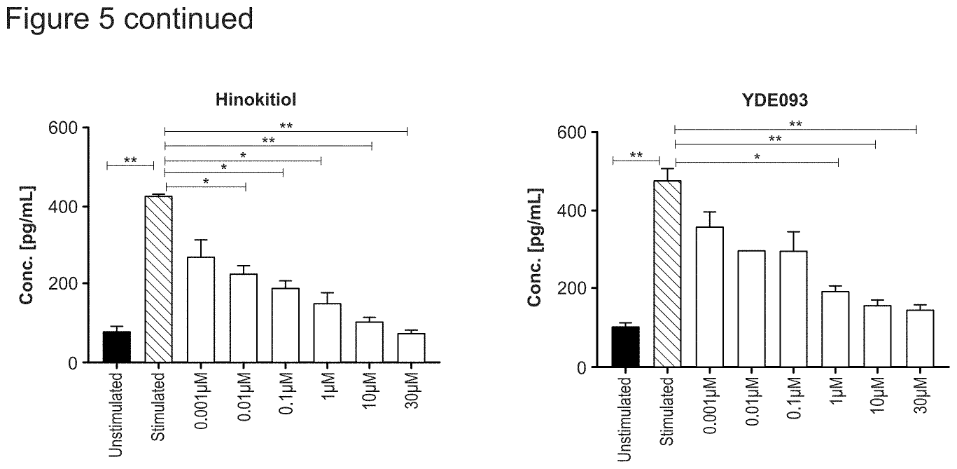

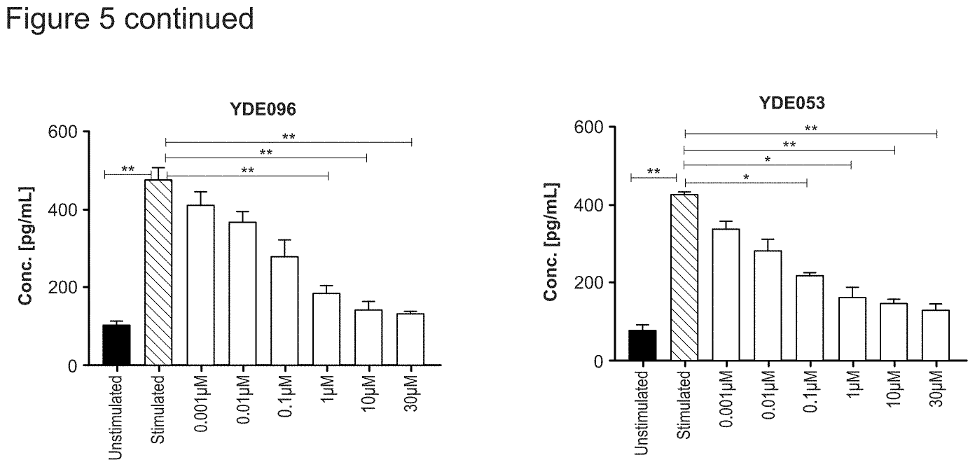

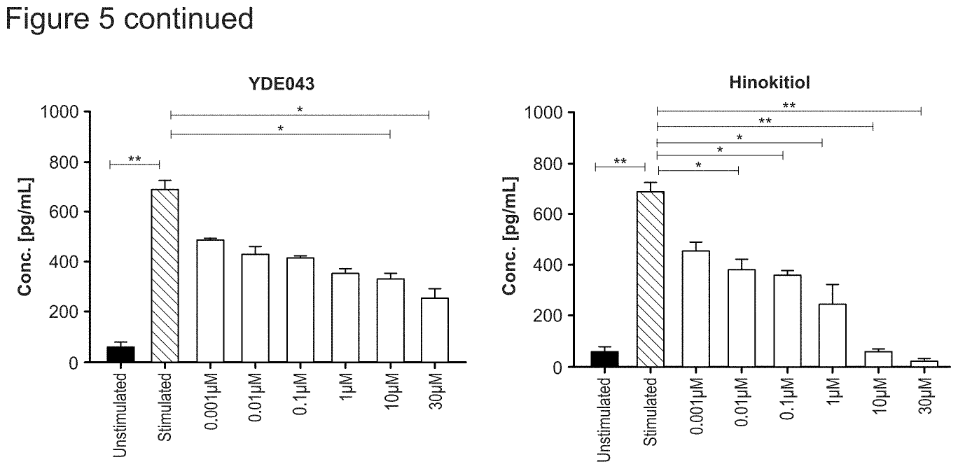

Results are shown in FIG. 5. Human corneal epithelial cells on stimulation with Poly I:C resulted in a significantly increase of IL-6 cytokine levels. Reference compound, Hinokitiol was found to significantly attenuate the Poly I:C stimulated IL-6 cytokine release in a dose dependent manner. Test compounds; YDE053, YDE048, YDE056, YDE057, YDE058, YDE067, YDE079, YDE011, YDE093, YDE096, YDE043 were found to attenuate Poly I:C induced of IL-6 cytokine release in a dose dependent manner. This was corroborated by the significant attenuation of IL-6 cytokine release at the tested concentrations that included; 30 μM, 10 μM, 1 μM and 0.1 μM respectively.

### Example 7: In Vitro Evaluation of Test Compounds for Apparent Permeability Using 21 Day Cultured Caco-2 Cell Monolayer

### Study Design & Materials & Methods

TABLE 2

| | |
|---|---|
| Cell origin | American Type Culture Collection |
| | (ATCC) |
| Test System | Caco-2 cells monolayer in 96 well format |
| Passage number | 36 |
| Test plate | Poly carbonate high pore density from |
| | Millipore with 0.4 μm pore size, 0.11 |
| | cm² active membrane area |
| Test compound concentration | 2 μM |
| Bidirectional assay (A-B & | 0 and 120 minutes |
| B-A) time points | |
| Buffer in Receiver wells | HBSS Buffer |
| Replicates | Two |
| End Point | Apparent permeability (Papp) |
| Bio analysis | LC-MS/MS |
| QC Compounds | Propranolol (High Permeable) and |
| | Atenolol (Low Permeable) |
| Assay Type | Bi-directional (A-B and B-A) |
| Cell Density | 12,000 cells/well (96 well plate) |

TABLE 3

| Test | | |
|---|---|---|
| Article | Compound | Molecular weight |
| 1 | YDE-011 | 953.10 |
| 2 | YDE-043 | 486.50 |

### Reagent Preparation

Preparation of (Dulbecco's Modified Eagles Medium) DMEM medium pH 7.4:

5 mL of 100 mM Sodium pyruvate, 5 mL of 100× non-essential amino acids, 5 mL of Penstrep was added to 100 mL of heat inactivated fetal bovine serum to 385 mL of DMEM aseptically and mixed thoroughly.

### Preparation of Hank's Balanced Salt Solution (HBSS) pH 7.4:

One vial of Hank's balanced salt (Sigma-H1387) was dissolved in 900 mL of milli Q water; pH was adjusted to 7.4 and made up the volume to 1000 mL of water. The solution was filter sterilized and store at 4° C.

### Preparation & Dilution of Test Compound:

10 mM stock solution of test compound was prepared in DMSO. 10 mM stock was diluted with 100% DMSO to prepare 0.2 mM, 0.2 mM stock was diluted with HBSS buffer to a final concentration of 2 μM.

### Assay Procedure

### Seeding of the Cells:

Add 250 μL of DMEM to the basal compartment of 96 well multi-screen Caco-2 plates and seed 12000 cells/well (0.16×10⁶ cells/ml) in all the apical wells required and 2 wells with only media as blank without cells. Place the Caco-2 plate in CO₂ incubator at 37° C. for proliferation of cells.

### Media Change:

The utilized media was replenished every alternate day by fresh medium. On 21st day, utilized medium was removed and washed twice with HBSS Buffer and incubated with HBSS buffer 30 min in incubator and initiated the assay.

### Apical to Basal Permeability:

75 μL of test compound was added to apical wells and 250 μL of HBSS buffer with 1% DMSO was added to basal wells. Samples were collected at 120 min and processed as stated below.

### Basal to Apical Permeability:

250 μL of test compound was added to basal wells and 75 μL of HBSS buffer with 1% DMSO was added to apical wells. Samples were collected at 120 min and processed as stated below.

### Sampling Processing:

Single point calibration curve in HBSS buffer was used. 100 μL of supernatant was diluted with 200 μL of water and submitted for LC-MS/MS analysis.

### Calculations:

Papp=dQ/dT×1/Co×1/A,

dQ is amount collected in the basolateral compartment of the 96 well filter plate;

dT is Time of incubation of drug on the cell monolayer;

Co is initial concentration of drug in the apical compartment of the well;

A is surface area of the filter.

Efflux ratio=Papp of basal to apical samples/Papp of apical to basal samples

Recovery: {(dQ of Apical+dQ of Basal)/Standard dQ}*100

### Results

The Caco-2 Permeability results of the test compounds is represented in below Table 4.

TABLE 4

| | Average Values | | | | | |
|---|---|---|---|---|---|---|
| | Papp (10⁻⁶ cm/sec) | | | | | |
| Compound | Apical to | Basal to | Efflux | A to B % | B to A % | |
| Name | Basal | Apical | Ratio | Recovery | Recovery | Classification |
| YDE-011 | 0.00 | 0.00 | NC | 15.00 | 88.50 | LOW |
| YDE-043 | 0.00 | 0.00 | NC | 34.00 | 67.25 | LOW |
| Propranolol | 23.83 | 37.07 | 1.56 | 80.07 | 90.30 | HIGH |
| Atenolol | 0.00 | 0.57 | NC | 93.90 | 85.33 | LOW |

NC: denotes Not calculated

TABLE 5
Data Interpretation: Criteria for classification
of the compounds (Caco-2 Permeability).

| Result of permeability Papp | |
|---|---|
| (10⁻⁶ cm/s) | Range |
| <1 | Low permeable |
| 1-10 | Medium permeable |
| >10 | High permeable |

### Example 8: Investigate the Effects of YY-101, YDE-011 & YDE-043 Compounds on Various Cytokine and Chemokine Release in Human Peripheral Blood Mononuclear Cells (PBMCs) (Study 1)

### Background and Purpose

The objective of this study was to investigate the effects of YY-101, YDE-011 and YDE-043 compounds on various cytokine and chemokine release in human peripheral blood mononuclear cells (hPBMCs) stimulated by known stimulants such as LPS, poly I:C or PMA/Ionomycin.

To achieve this goal, a pilot study was conducted to establish a dose and time-dependent IL-6 cytokine release in hPBMCs by each stimulant. Upon selection of the dose and time point for the best stimulant, the main study was conducted to establish the efficacious dose-response curve of YY-101, YDE-011, YDE-043 on up to 30 cytokine and chemokine release in hPBMC induced by LPS.

### Materials & Methods

### Reagents and Solutions

Human PBMCs (ATCC, USA)
Lipopolysaccharides from Escherichia coli (Sigma Aldrich, USA)
poly (I:C) (Sigma Aldrich, USA)
PMA (Sigma Aldrich, USA)
Ionomycin (Sigma Aldrich, USA)
Human IL-6 ELISA kit (Thermo fisher, USA)
Human magnetic Luminex assay kit (R&D System, USA)
Xiidra® (Shire, USA)
Test materials (YY-101, YDE-011, YDE-043) were stored in a deep freezer (−20° C.).

### Cell Culture

Human PBMCs frozen in a cryopreserve was thawed, washed with Hank's Balanced Salt Solution containing 10% Fetal bovine serum, and seeded onto a 24-well plate at a density of 1×10⁶ cells/well with culture RPMI media containing 10% Fetal bovine serum in growth media in a 24 well plate for 12, 24, and 48 hrs and maintained at 37° C. in an atmosphere of 95% air and 5% CO₂.

### 1^{st} Pilot Study

Human PBMCs was pretreated with a reference compound; Xiidra® (the final concentration @ 10 uM) for 2 h before stimulation. An equal volume of 0.5% DMSO was used as a vehicle control. Then, the cells were treated with stimulant(s), poly(I:C) at 25 ug/ml, LPS at 5 and 25 ug/ml at and PMA (5 ng/ml)/Ionomycin (1 ug/ml). Triplicates of 100 ul of supernatants from treated cells was harvested at 12, 24, and 48 h and placed in 96 well for Human IL-6 ELISA measurement according to the manufacturer's instructions (Invitrogen, CA, USA).

The pilot study arm with triplicate wells per group in a 24-well plate:
1. PBMC: 0.5% DMSO
2. PBMC+Poly I:C (25 ug/ml)
3. PBMC+LPS (5 ug/ml)
4. PBMC+LPS (25 ug/ml)
5. PBMC+PMA (5 ng/ml)/Ionomycin (1 ug/ml)
6. PBMC+Poly I:C (25 ug/ml)+Xiidra® 10 uM
7. PBMC+LPS (5 ug/ml)+Xiidra® 10 uM
8. PBMC+LPS (25 ug/ml)+Xiidra® 10 uM
9. PBMC+PMA (5 ng/ml)/Ionomycin (1 ug/ml)+Xiidra® 10 uM

### 2^{nd} Pilot Study

Human PBMCs was pretreated with a test compound; YDE-011 at 30 uM, 1 μM for 2 h before stimulation. An equal volume of 0.5% DMSO was used as a vehicle control. Then, the cells were treated with stimulants, poly(I:C) at 25 ug/ml, LPS at 5 ug/ml at and PMA (5 ng/ml)/Ionomycin (1 ug/ml). The supernatants from treated cells were harvested at 24 h and placed in 96 well for Human IL-6 ELISAS measurement according to the manufacturer's instructions (Invitrogen, CA, USA).

The pilot study arm with duplicate wells per group in a 24-well plate:
1. PBMC: 0.5% DMSO
2. PBMC+Poly I:C (25 ug/ml)
3. PBMC+LPS (5 ug/ml)
4. PBMC+PMA (5 ng/ml)/Ionomycin (1 ug/ml)
5. PBMC+Poly I:C (25 ug/ml)+Xiidra® 30 uM
6. PBMC+LPS (5 ug/ml)+Xiidra® 30 uM
7. PBMC+PMA (5 ng/ml)/Ionomycin (1 ug/ml)+Xiidra® 30 Um
8. PBMC+Poly I:C (25 ug/ml)+Xiidra® 1 uM
9. PBMC+LPS (5 ug/ml)+Xiidra® 1 uM
10. PBMC+PMA (5 ng/ml)/Ionomycin (1 ug/ml)+Xiidra® 1 uM

### Human IL-6 ELISA

IL-6 measurement was performed by the sandwich ELISA method. The capture antibody was coated on a 96-well plate (Corning™ Costar™ 9018, NY, USA) at 100 ul/well and incubated overnight at 4° C. The interaction was blocked at room temperature for 2 h to prevent non-specific binding of the antigen-antibody. Then, the plate was incubated overnight at 4° C. with 1:200 diluted cell supernatants and standard dilute serial dilutions at 100 ul/well. Detection antibody was dispensed into the plate at 100 ul/well, and then allowed to incubate at a room temperature for 1 h. Finally, streptavidin-HRP (100 ul/well) was incubated at room temperature for 30 min. TMB substrate (100 ul/well) was added and allowed to incubate for 15 min until colored reaction was observed in the dark condition. The sample reading was measured at 450 nm wavelength and 570 nm, and the quantification of the sample was converted as concentration (ng/ml) based on the standard curve extrapolation.

### The Main Study

Human PBMCs were pretreated with YY-101, YDE-011, YDE-043 or reference compound; Xiidra® at various concentration (ranging from 5 μM to 500 nM) for 2 h before stimulation. An equal volume of 0.5% DMSO was used as a vehicle control. Then, the cells were treated with LPS at 5 ug/ml, which was selected based on the pilot study. The supernatants from treated cells were harvested at 24 h and placed in a 96 well for Luminex cytokine & chemokine profiling using a Luminex 200 multiplex assay (Luminex; R&D system, USA).

The main study arm with triplicate wells per group in a 24-well plate:
1. Blank
2. PBMC: 0.5% DMSO
3. PBMC+LPS (5 ug/ml)
4. PBMC+YY-101 500 nM
5. PBMC+YY-101 50 nM
6. PBMC+YY-101 5 nM
7. PBMC+YY-101 0.5 nM
8. PBMC+LPS (5 ug/ml)+Xiidra® 500 nM
9. PBMC+LPS (5 ug/ml)+Xiidra® 50 nM
10. PBMC+LPS (5 ug/ml)+Xiidra® 5 nM
11. PBMC+LPS (5 ug/ml)+Xiidra® 0.5 nM
12. PBMC+LPS (5 ug/ml)+Xiidra® 0.05 nM
13. PBMC+LPS (5 ug/ml)+Xiidra® 0.005 nM
14. PBMC+LPS (5 ug/ml)+YY-101 500 nM
15. PBMC+LPS (5 ug/ml)+YY-101 50 nM
16. PBMC+LPS (5 ug/ml)+YY-101 5 nM
17. PBMC+LPS (5 ug/ml)+YY-101 0.5 nM
18. PBMC+LPS (5 ug/ml)+YY-101 0.05 nM
19. PBMC+LPS (5 ug/ml)+YY-101 0.005 nM
20. PBMC+LPS (5 ug/ml)+YDE-011 500 nM
21. PBMC+LPS (5 ug/ml)+YDE-011 50 nM
22. PBMC+LPS (5 ug/ml)+YDE-011 5 nM
23. PBMC+LPS (5 ug/ml)+YDE-011 0.5 nM
24. PBMC+LPS (5 ug/ml)+YDE-011 0.05 nM
25. PBMC+LPS (5 ug/ml)+YDE-011 0.005 nM
26. PBMC+LPS (5 ug/ml)+YDE-043 500 nM
27. PBMC+LPS (5 ug/ml)+YDE-043 50 nM
28. PBMC+LPS (5 ug/ml)+YDE-043 5 nM
29. PBMC+LPS (5 ug/ml)+YDE-043 0.5 nM
30. PBMC+LPS (5 ug/ml)+YDE-043 0.05 nM
31. PBMC+LPS (5 ug/ml)+YDE-043 0.005 nM

### Multiplex Assay

Multiplex assay was used to measure 30 cytokines and chemokines. The 1:200 diluted cell supernatant and serial dilution of the standard were dispensed into a 96 well plate at 50 ul/well. Then, the pre-mixed cocktail of antibody-coated magnetic beads was dispensed at 50 ul/well and incubated at room temperature for 2 h in a horizontal orbital microplate shaker at 800±500 rpm. The beads were washed using a magnetic device to prevent loss. The biotin-antibody was dispensed in 50 ul of each well and incubated at room temperature for 1 h in a shaker at 800±500 rpm. After washing, streptavidin-PE was added at 50 ul/well and incubated at room temperature for 30 min in a shaker under the same conditions. Finally, after washing, wash buffer (100 ul/well) was added to the plate and incubated in a shaker for 2 min. The Luminex™ 200 setting were set according to the manufacturer's protocol. The data was calculated with a standard five-parameter logistic nonlinear regression analysis of the data (xPonent software 4.2, USA). Results were presented as concentration (ng/ml or pg/ml).

Below is the list of cytokines and chemokines either in 26-plex or 4-plex.

TABLE 6

| | Cytokine/Chemokine |
|---|---|
| Human | list |
| 26 plex | |
| 1 | MCP-1 (CCL2) |
| 2 | MIP-1 alpha (CCL3) |
| 3 | MIP-3 alpha (CCL20) |
| 4 | Fractalkine (CX3CL1) |
| 5 | Fas-L |
| 6 | GM-CSF |
| 7 | ICAM-1 |
| 8 | IFN-gamma |
| 9 | IL-1 alpha |
| 10 | IL-1 beta |
| 11 | IL-2 |
| 12 | IL-4 |
| 13 | IL-5 |
| 14 | IL-6 |
| 15 | IL-8 (CXCL8) |
| 16 | IL-10 |
| 17 | IL-13 |
| 18 | IL-17A |
| 19 | Leptin |
| 20 | MMP-3 |
| 21 | MMP-8 |
| 22 | MMP-9 |
| 23 | RAGE |
| 24 | L-Selectin |
| 25 | TNF-alpha |
| 26 | VEGF-D |
| 4 plex | |
| 1 | MIP-1 beta (CCL4) |
| 2 | RANTES (CCL5) |
| 3 | TIMP-1 |
| 4 | VEGF-A |

### Compound Delivery & Formulation

The concentration of compound was prepared according to the information provided by the client.

All compound was serially diluted in from the stock concentrations and the final concentration of DMSO was not exceed 0.5% DMSO. All compounds were prepared on the same day of the treatment.

### Statistical Analysis

All values are presented as mean±standard error of mean (SEM). The statistical significance of the results was analyzed using one-way ANOVA with a Bonferroni post hoc. The statistical analyses were performed using the SPSS software (SPSS 22.0, USA). Each compound treated PBMCs was compared to that of the stimulant induced PBMCs. The significant threshold was fixed at 0.05 i.e. p value has to be lower than 0.05 to be significant.

### Results

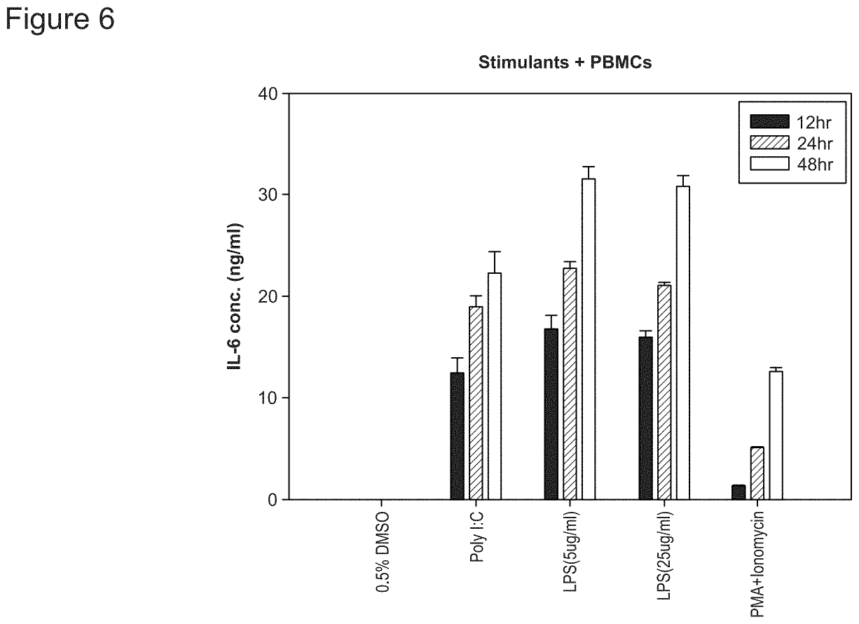

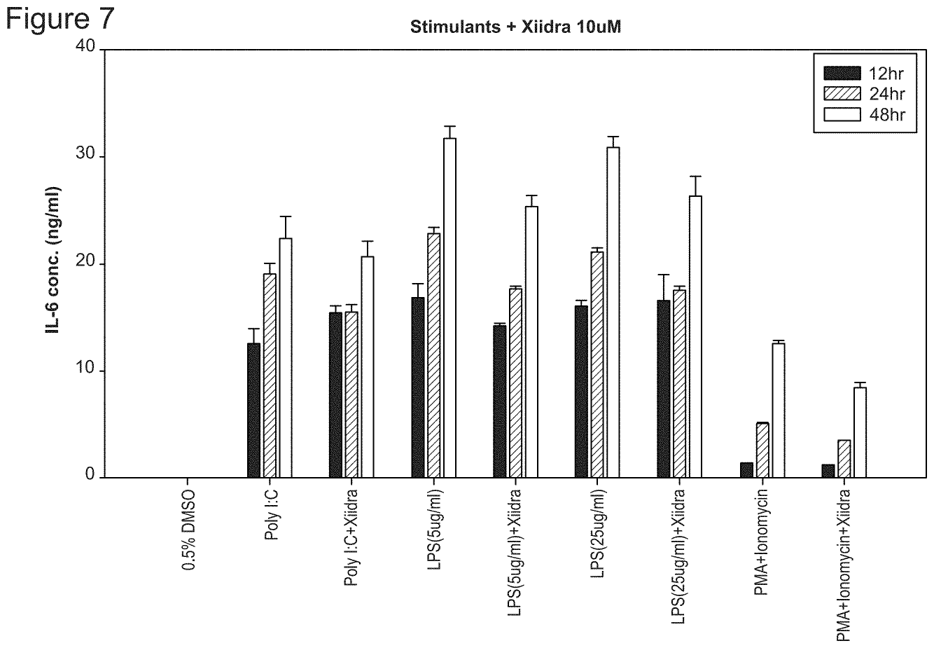

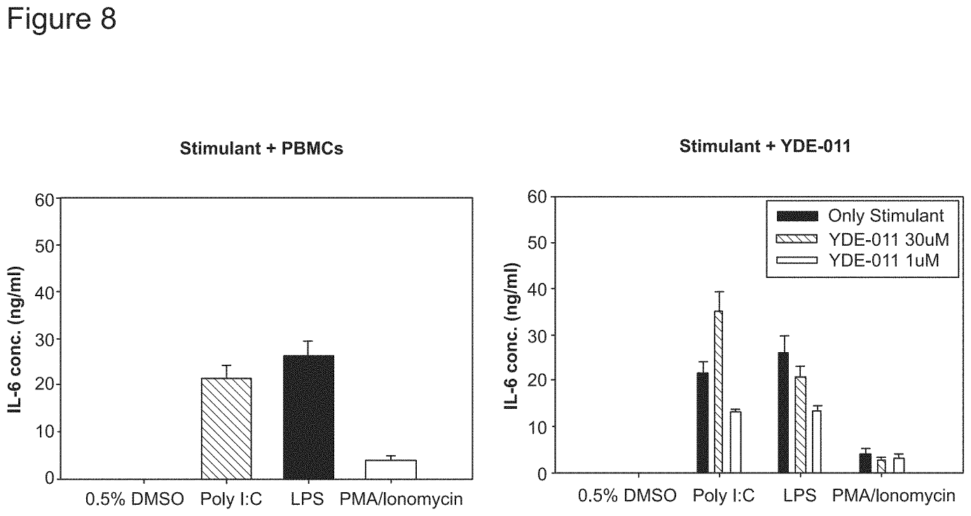

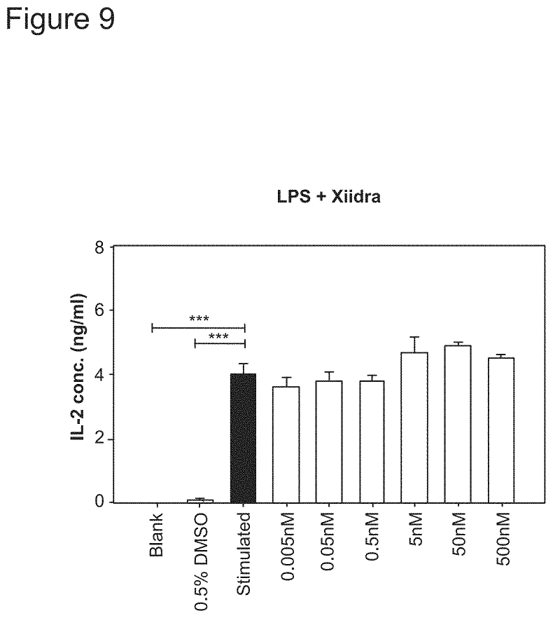

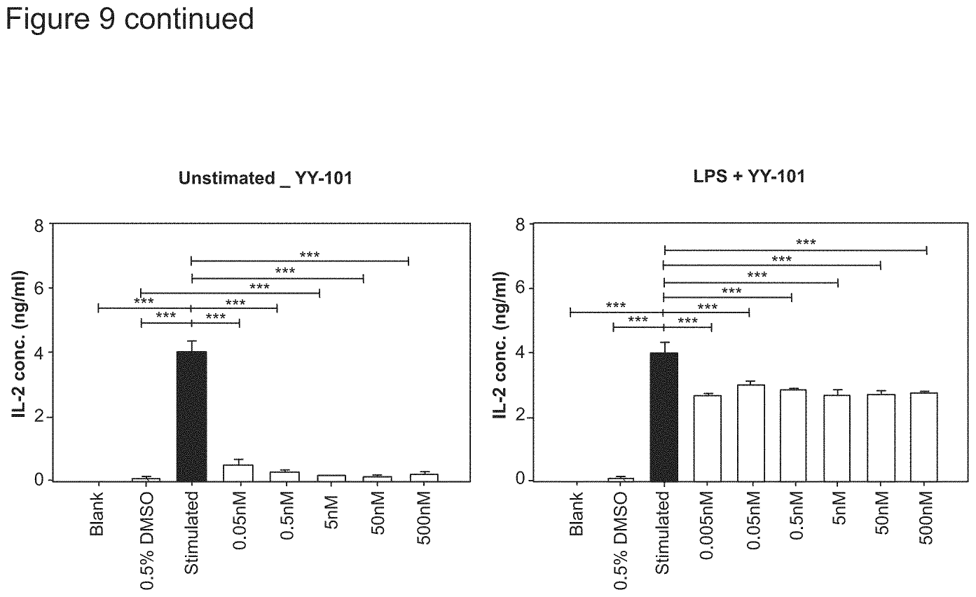

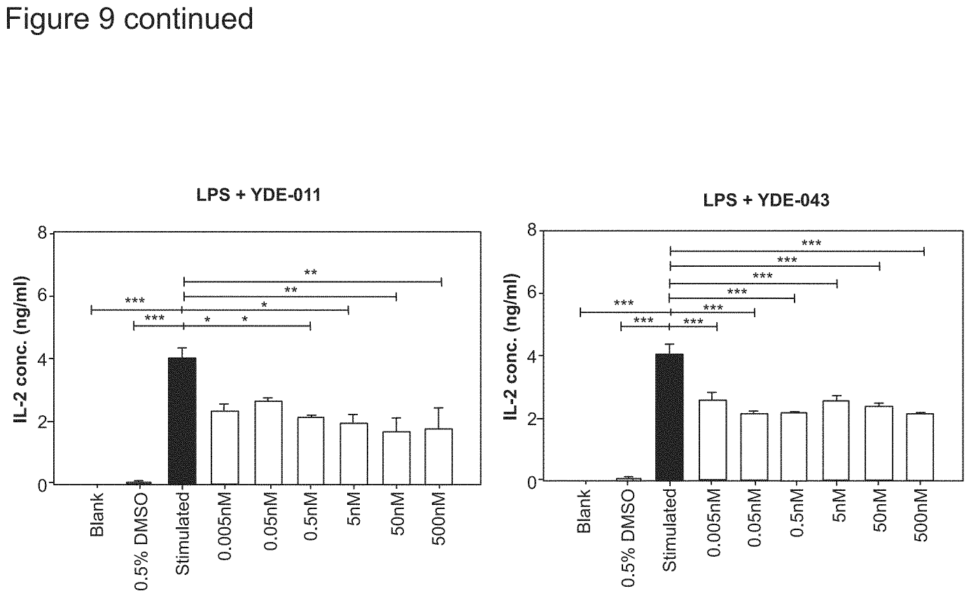

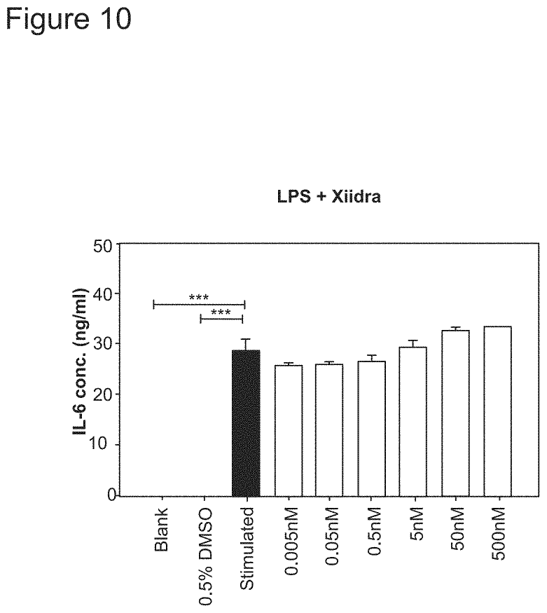

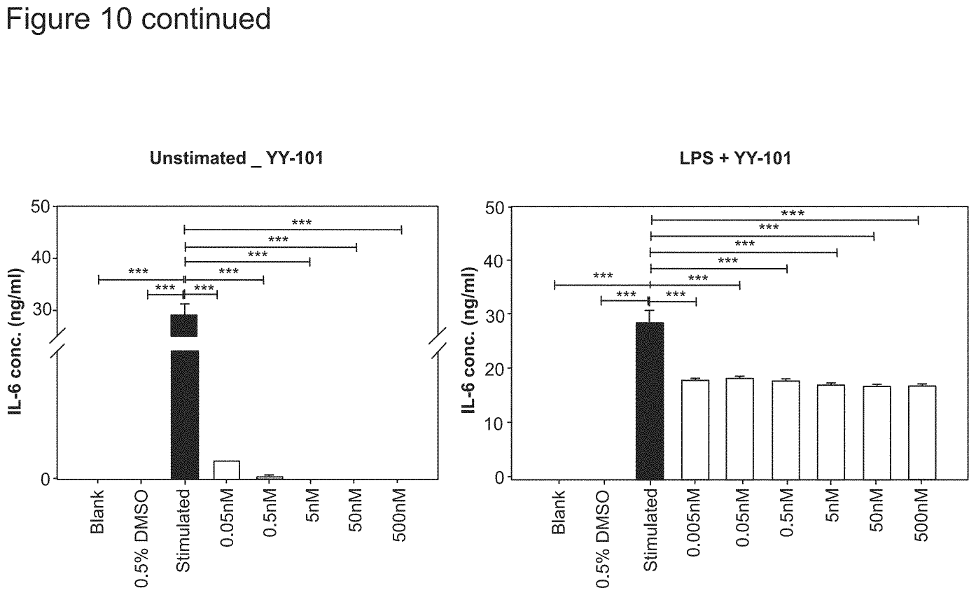

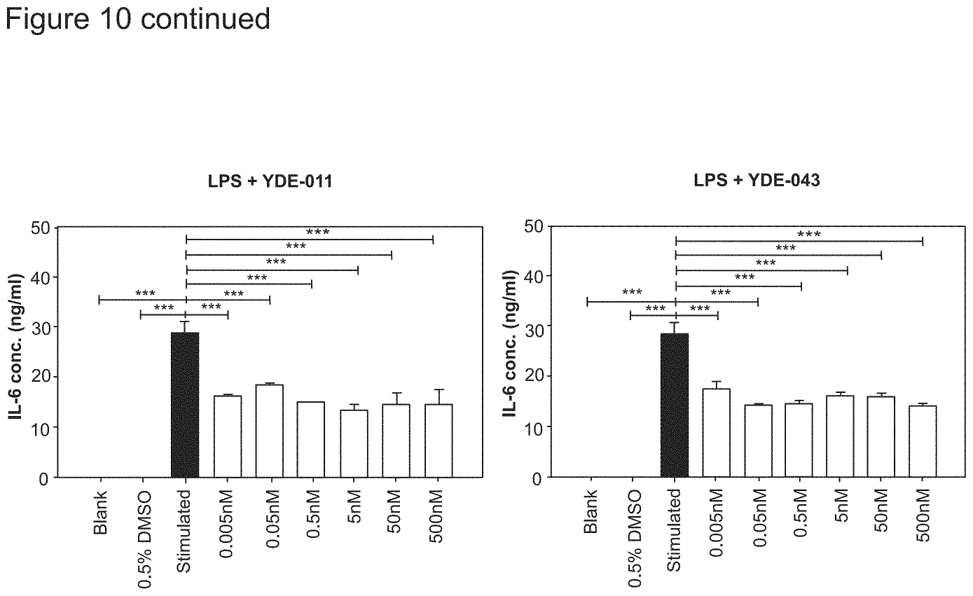

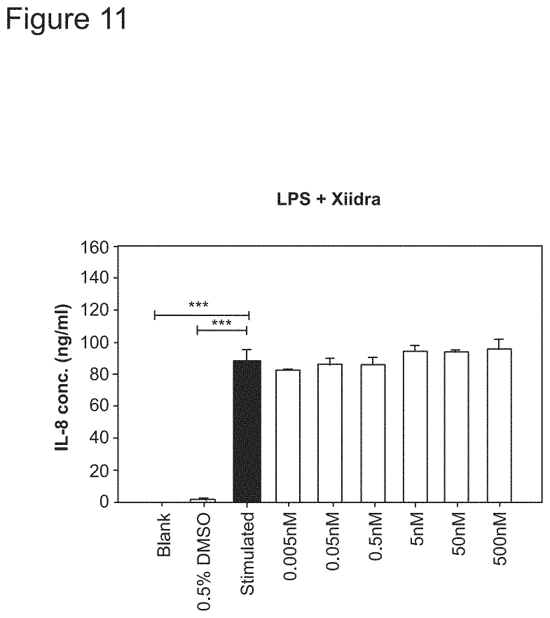

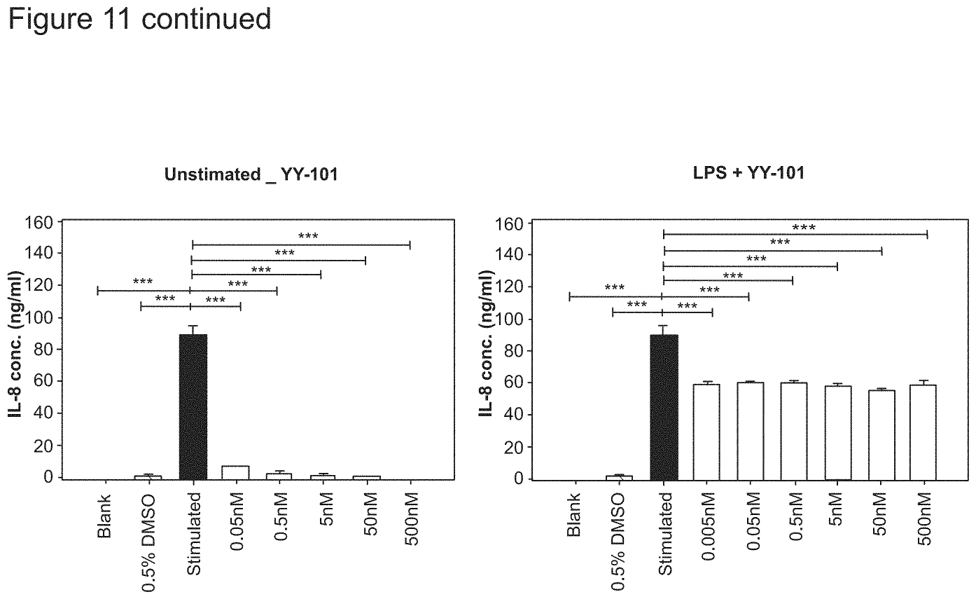

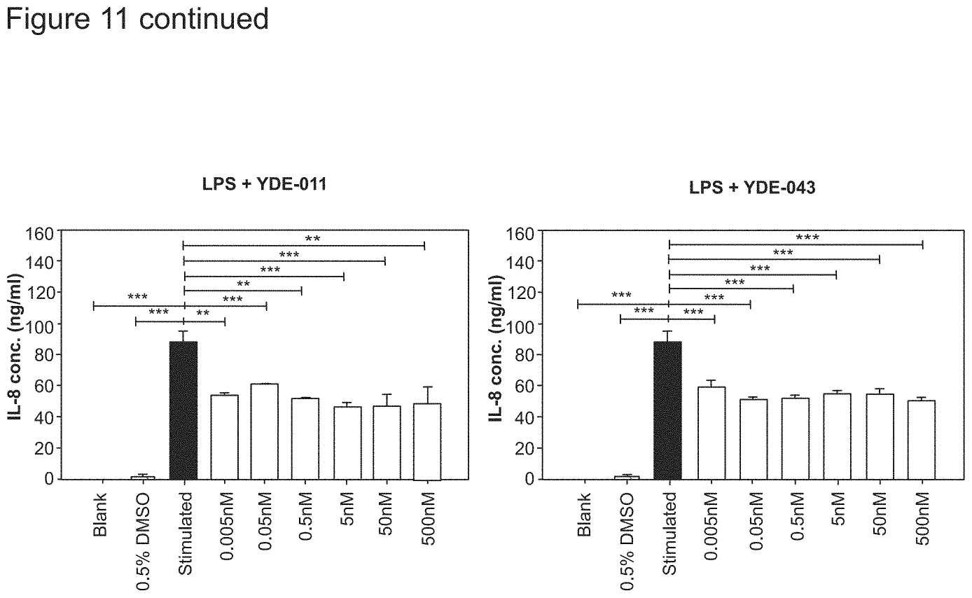

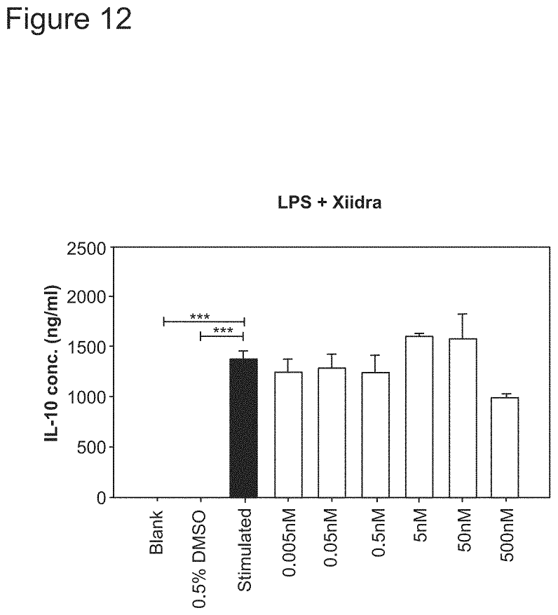

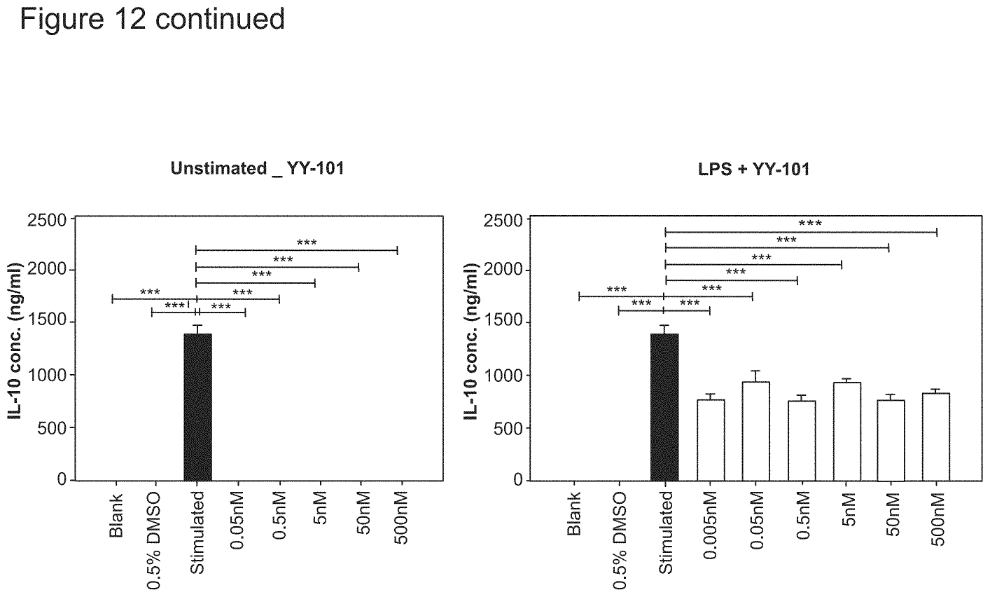

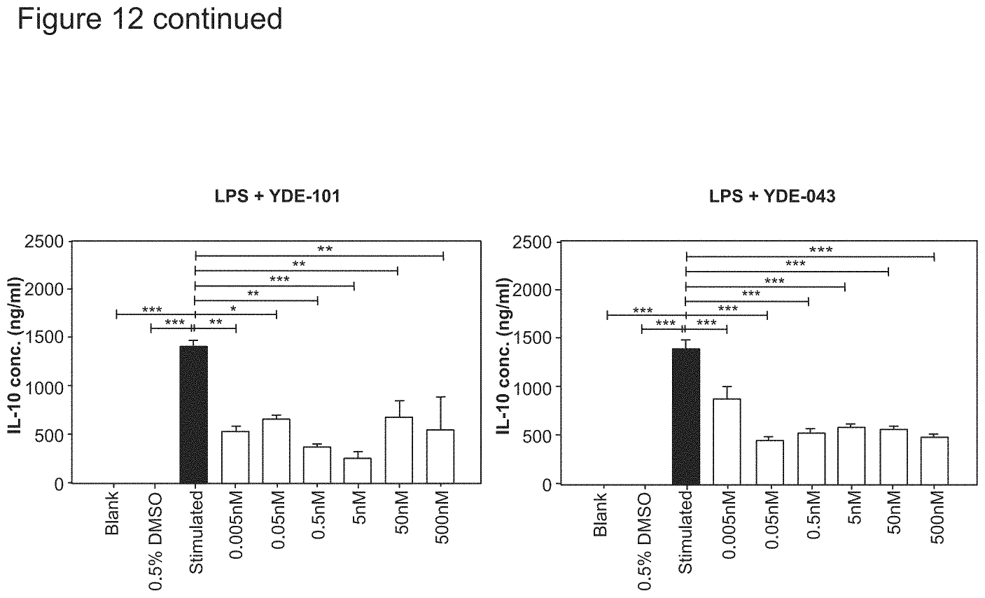

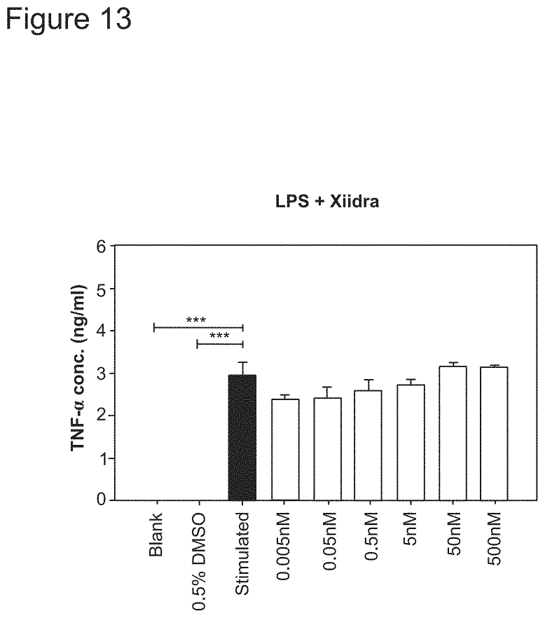

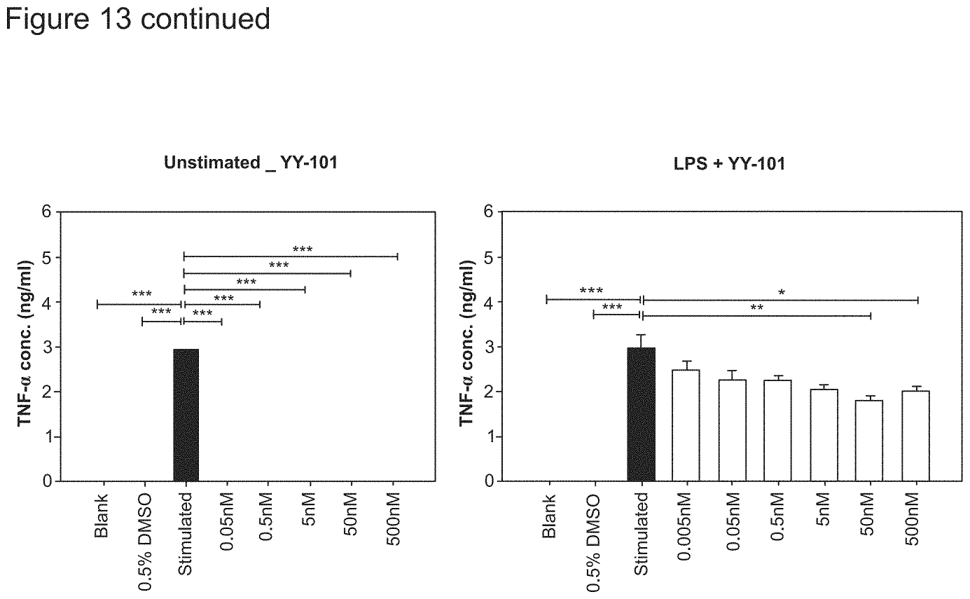

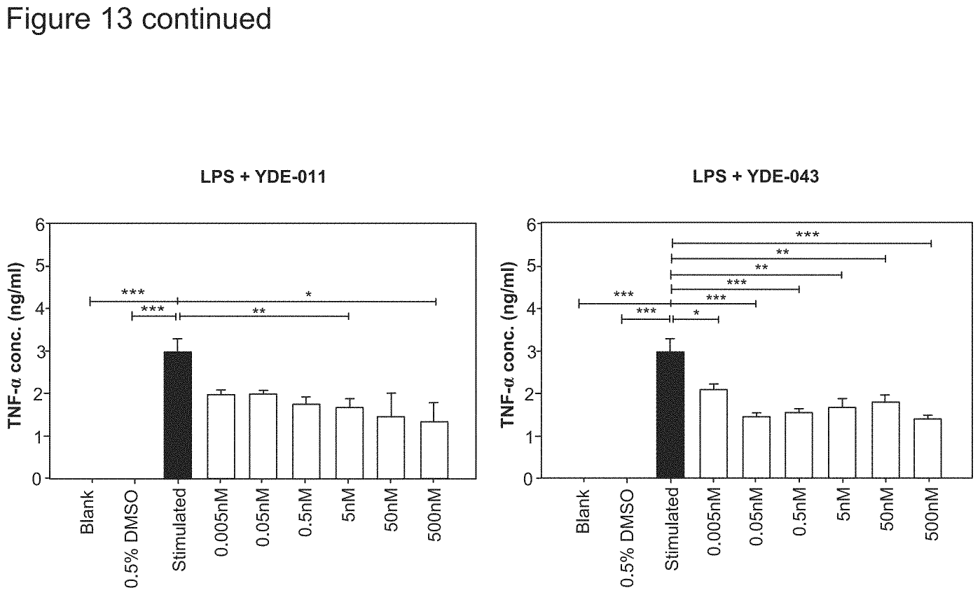

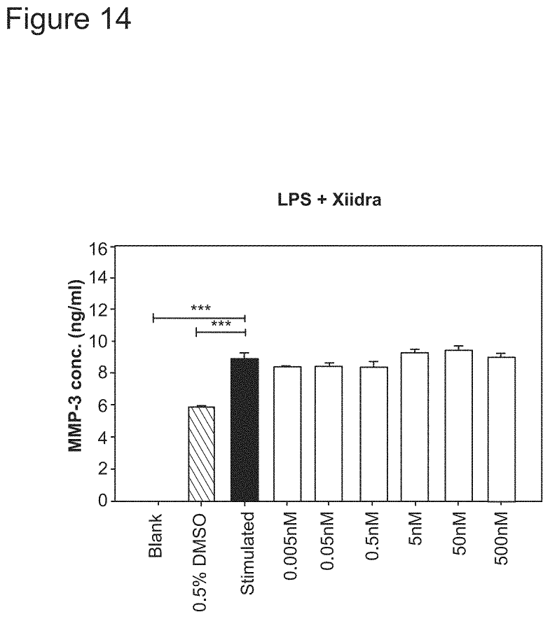

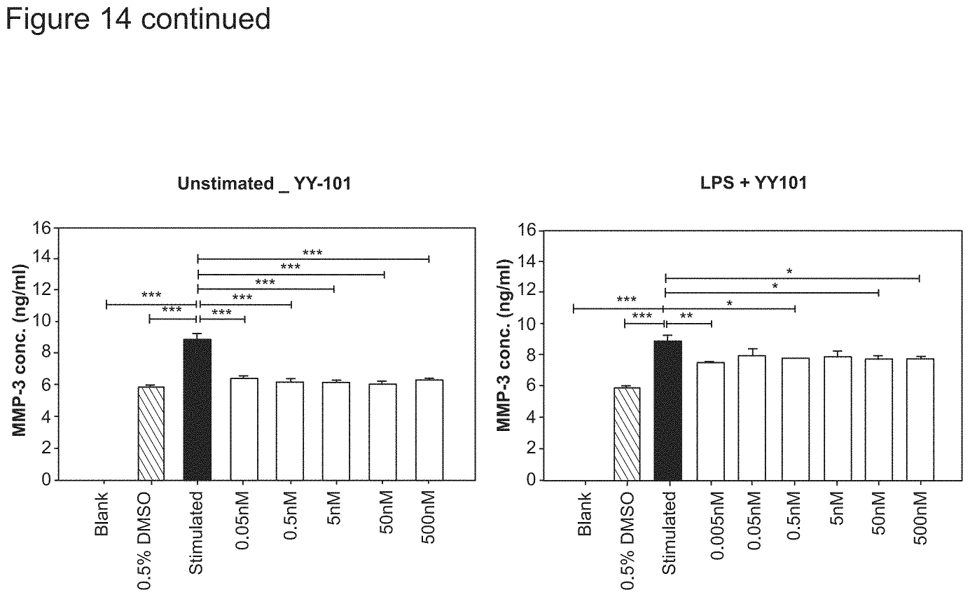

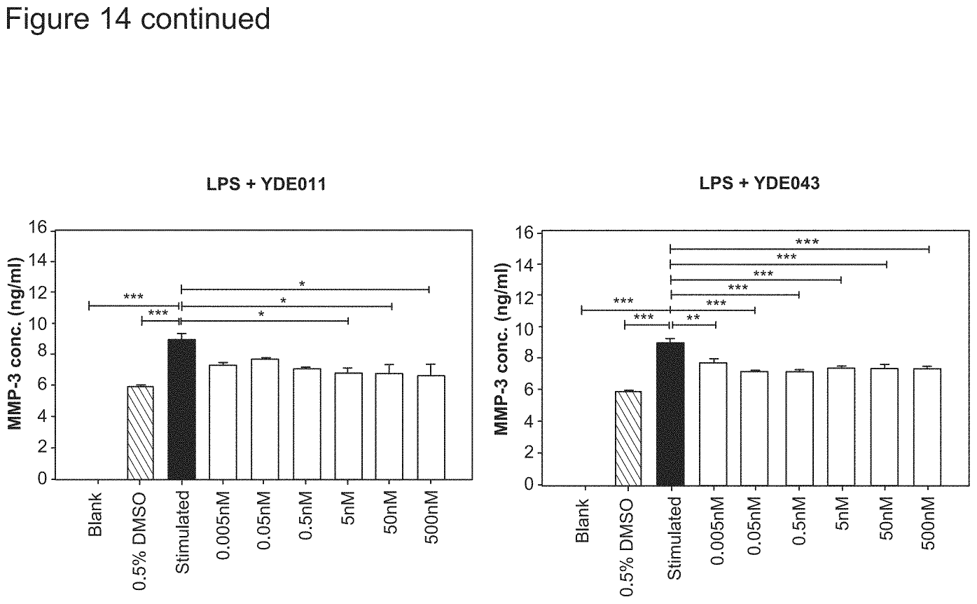

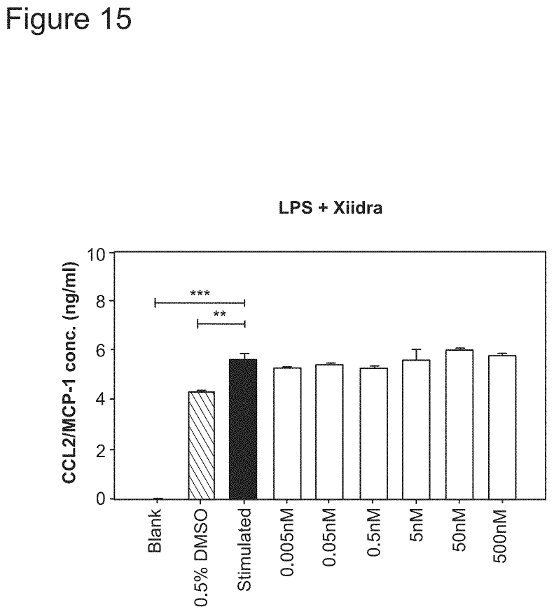

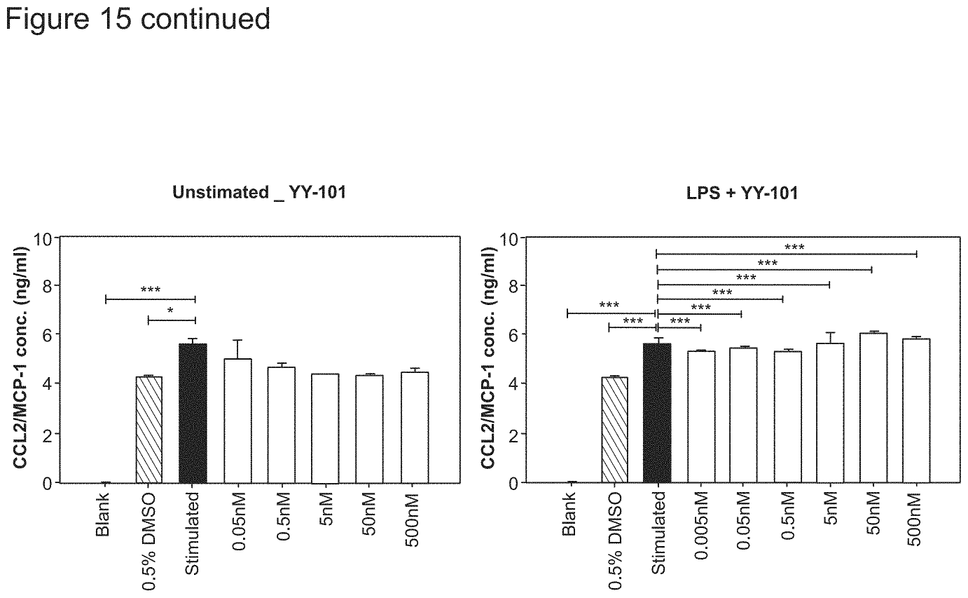

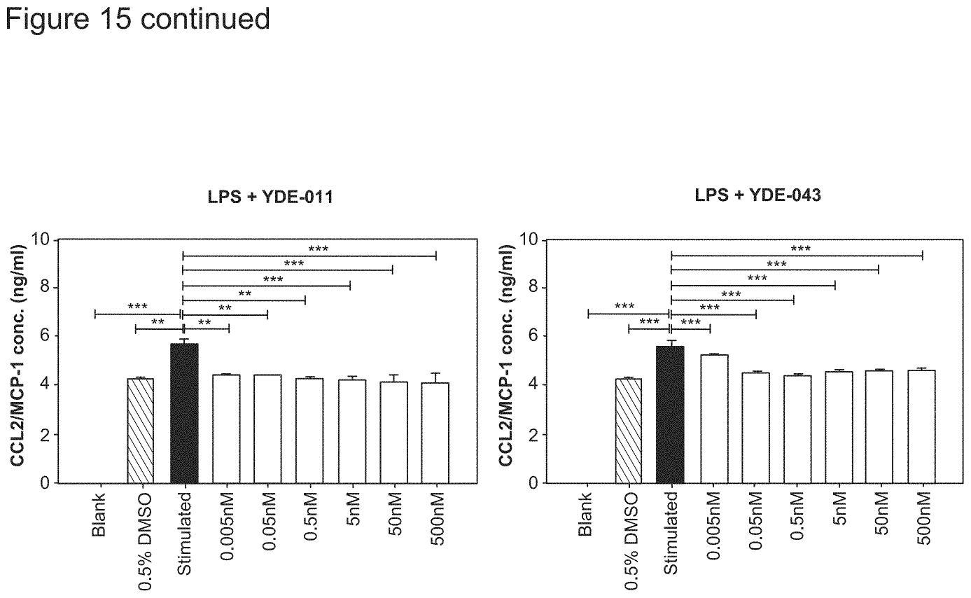

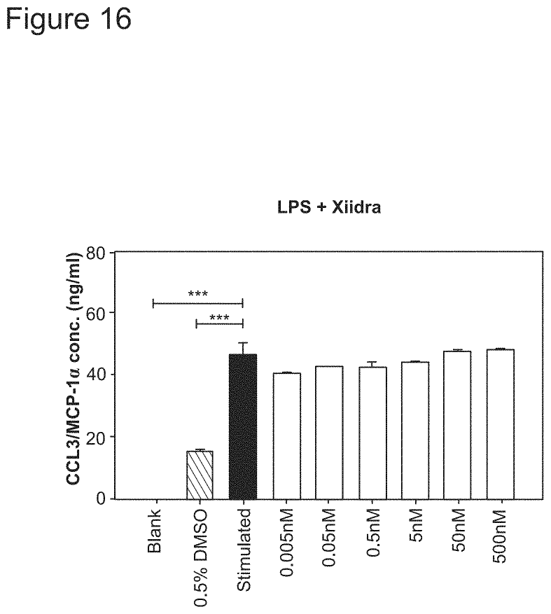

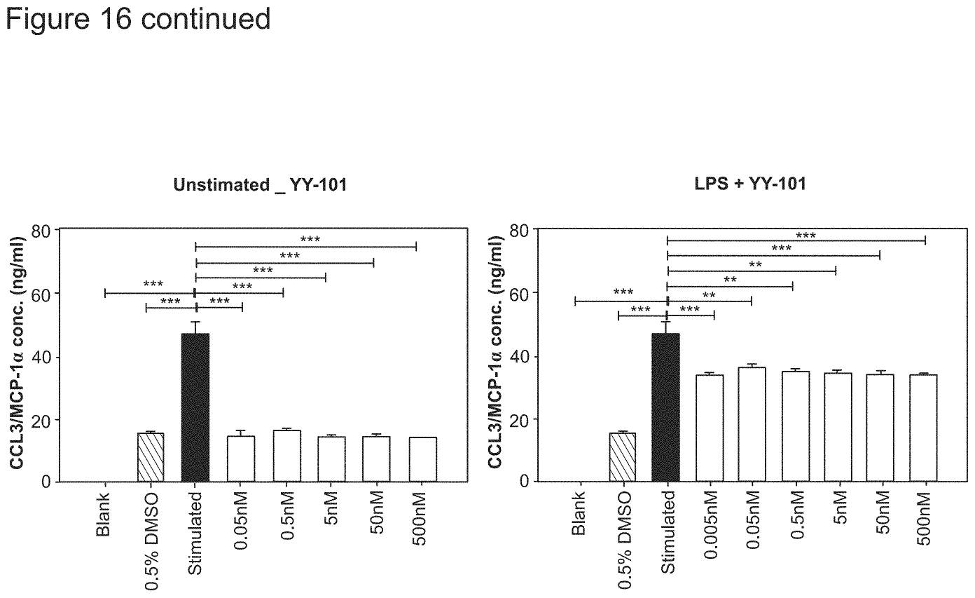

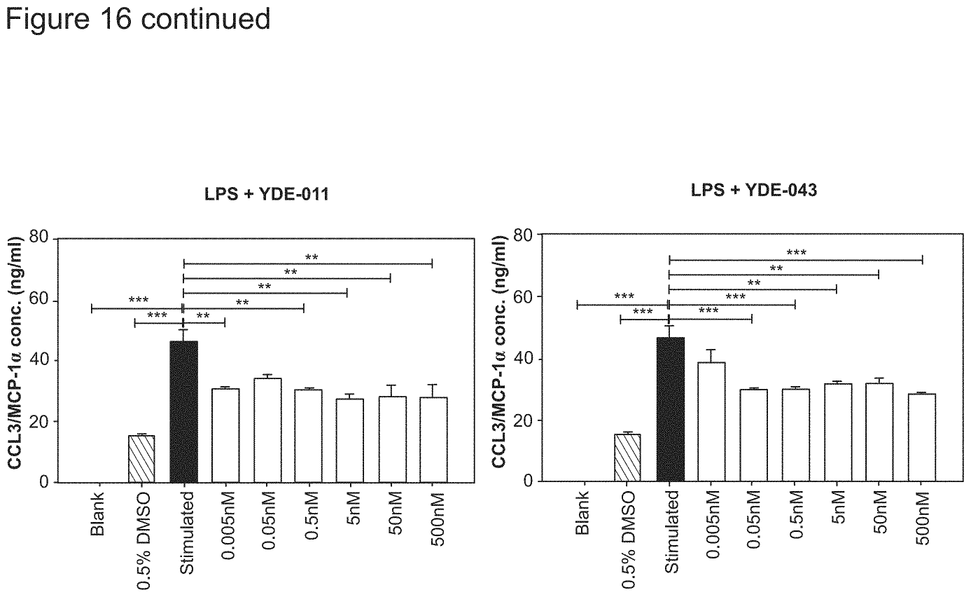

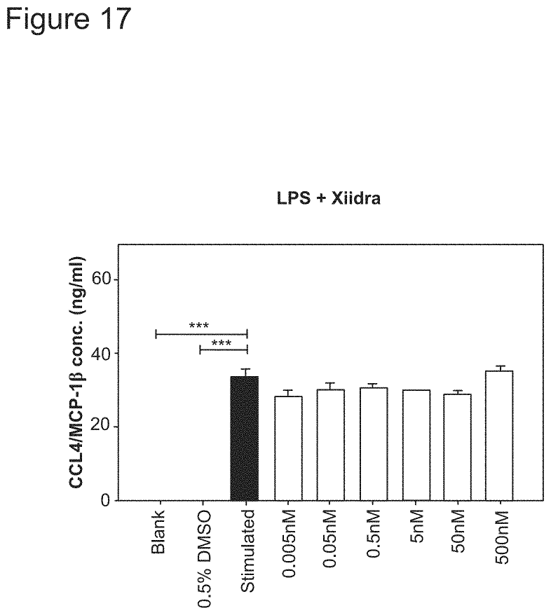

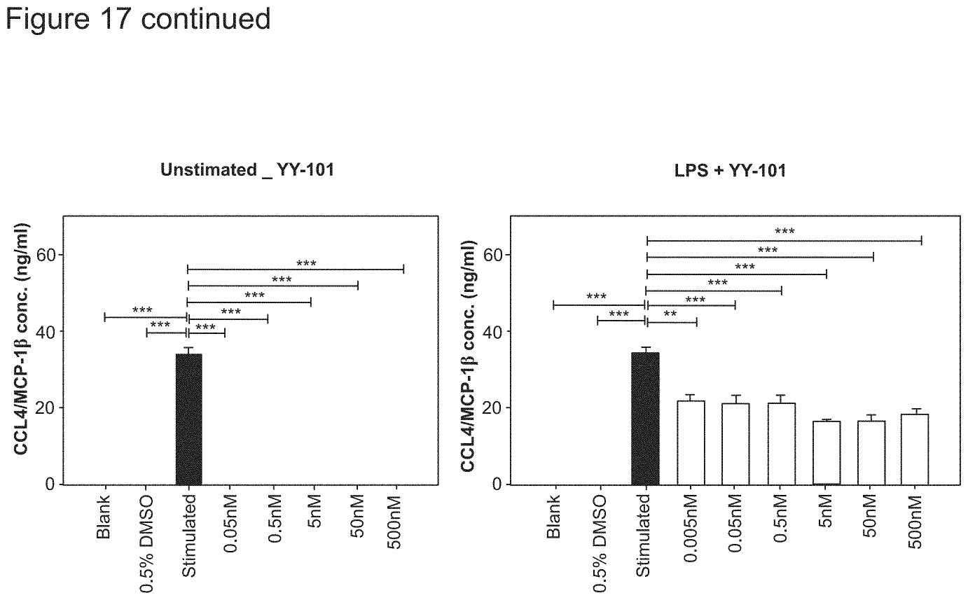

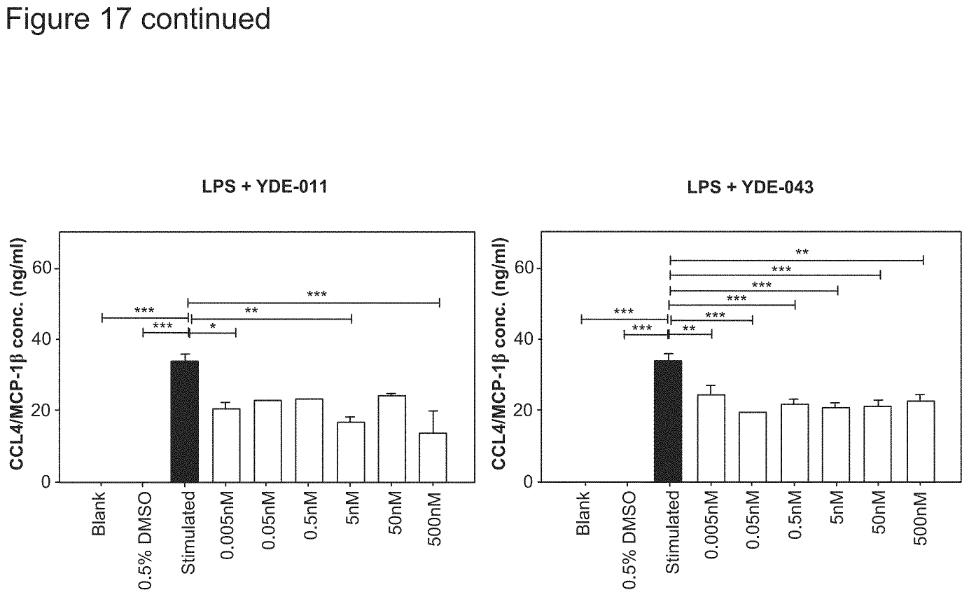

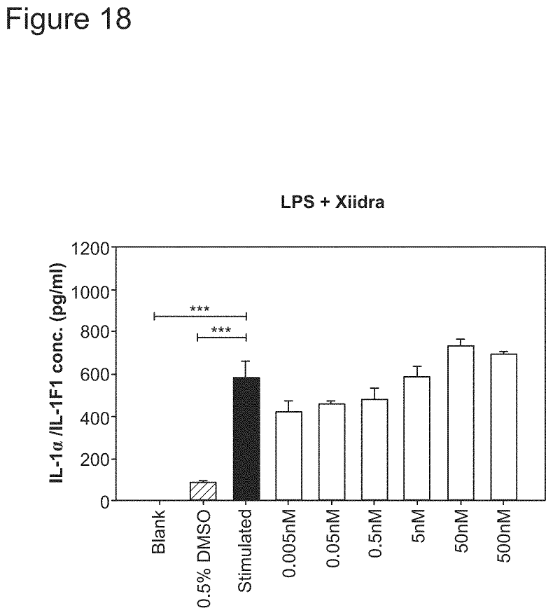

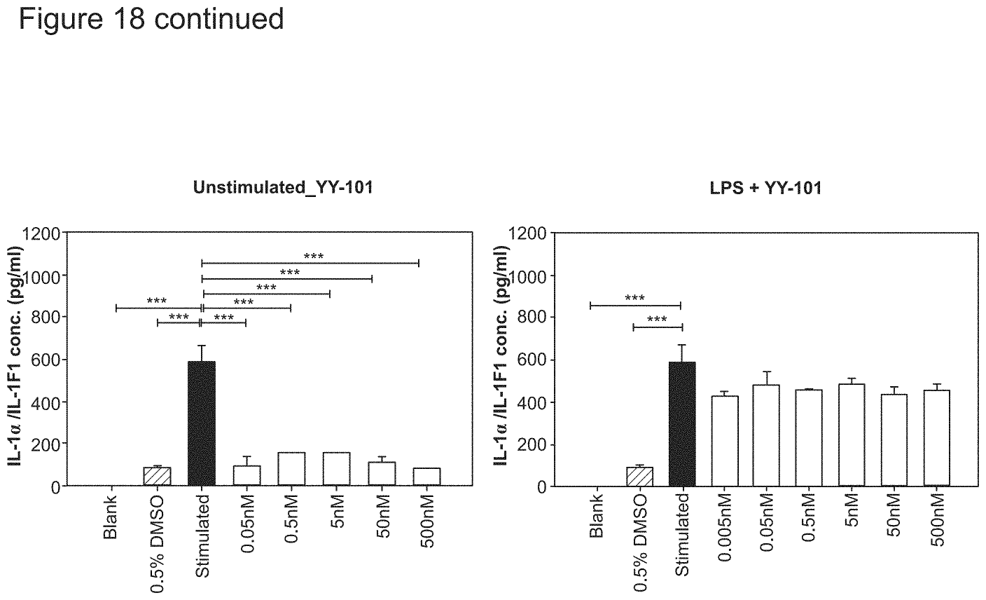

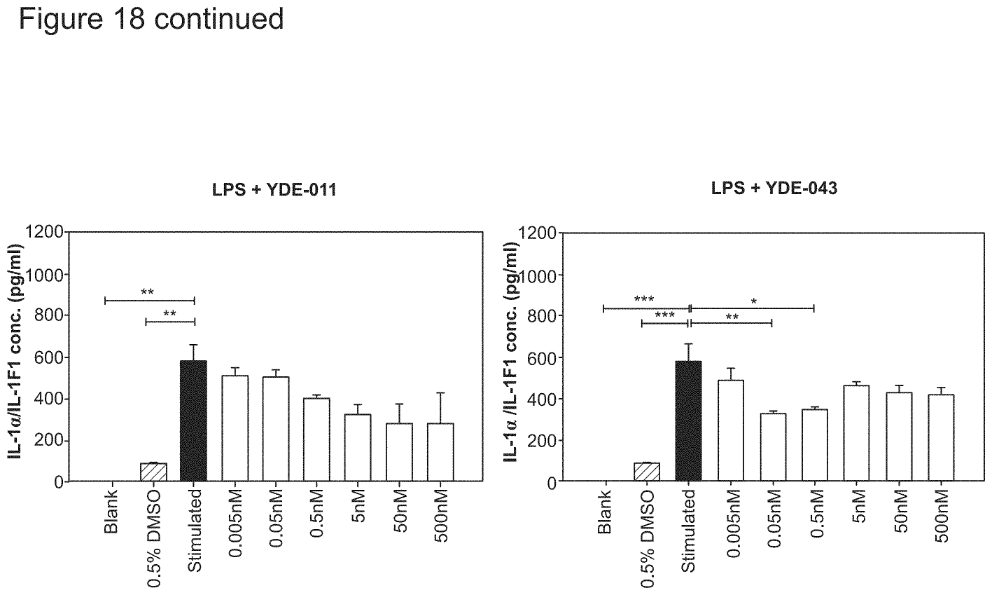

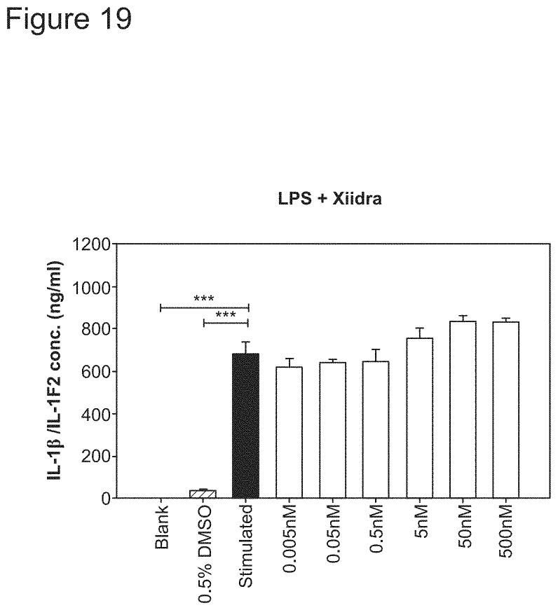

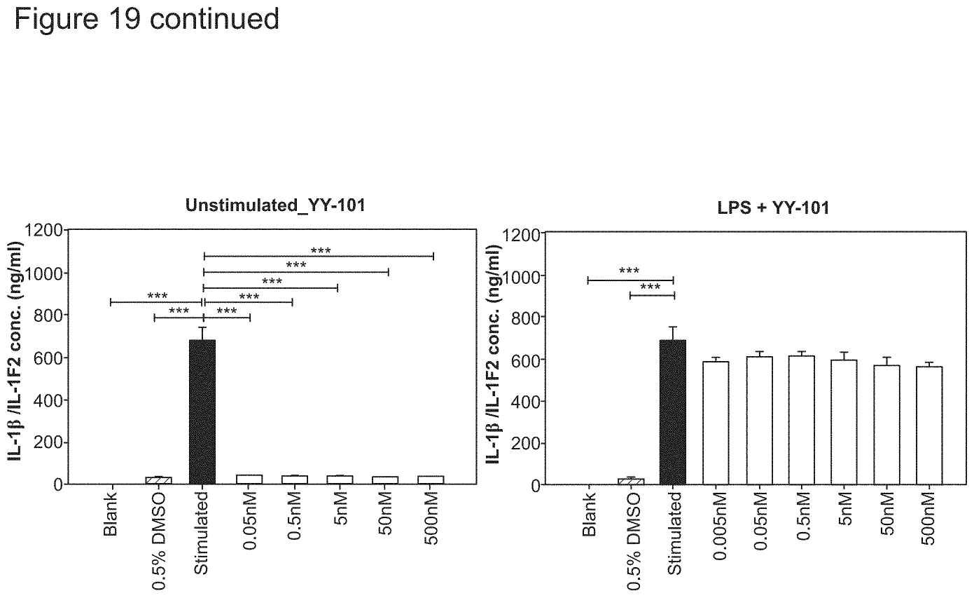

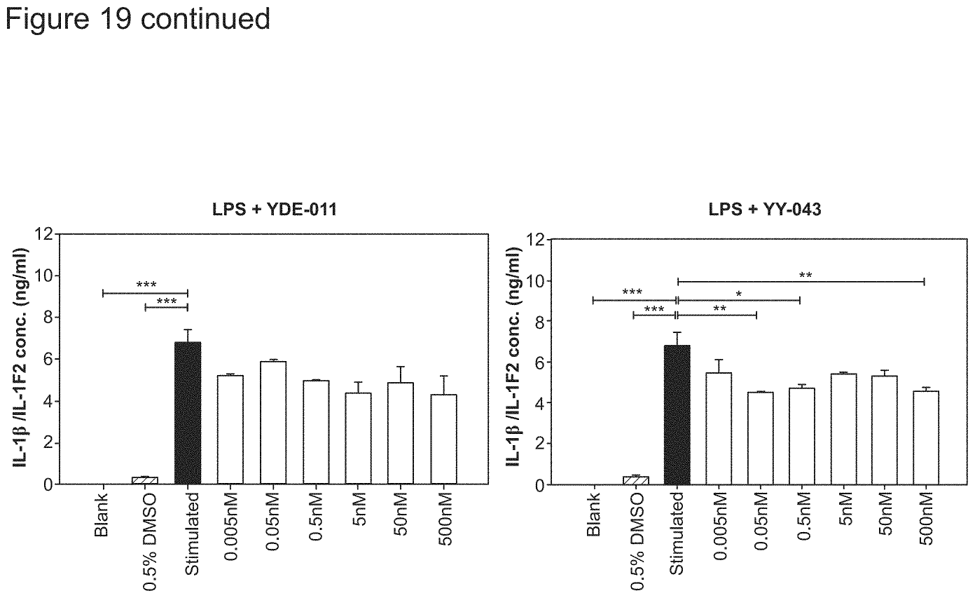

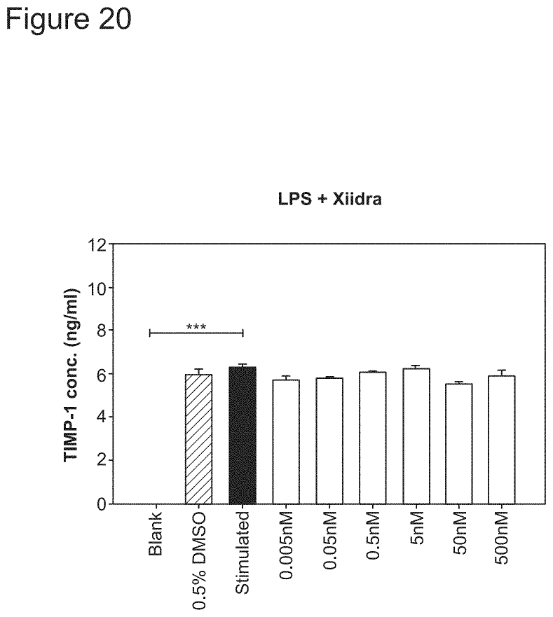

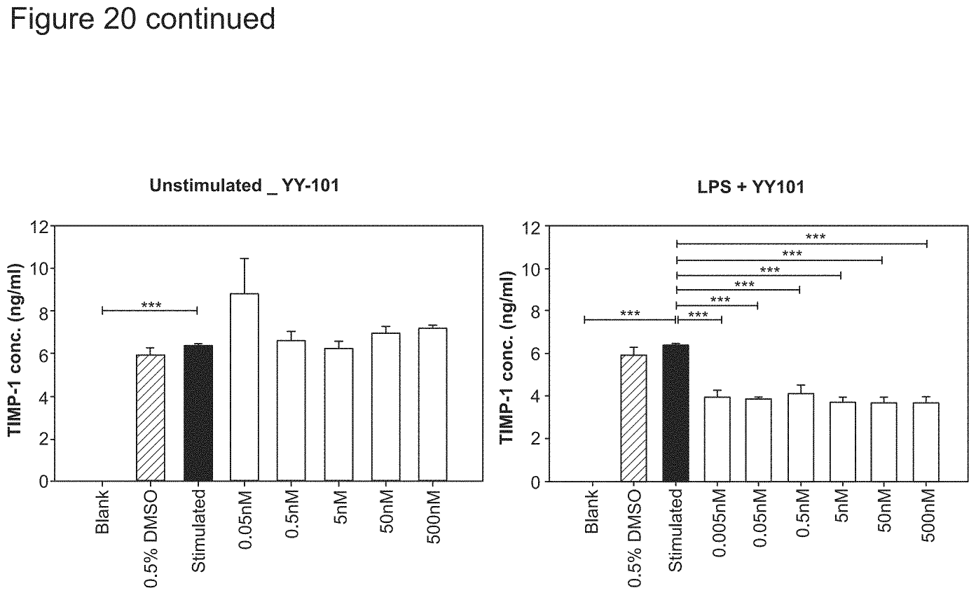

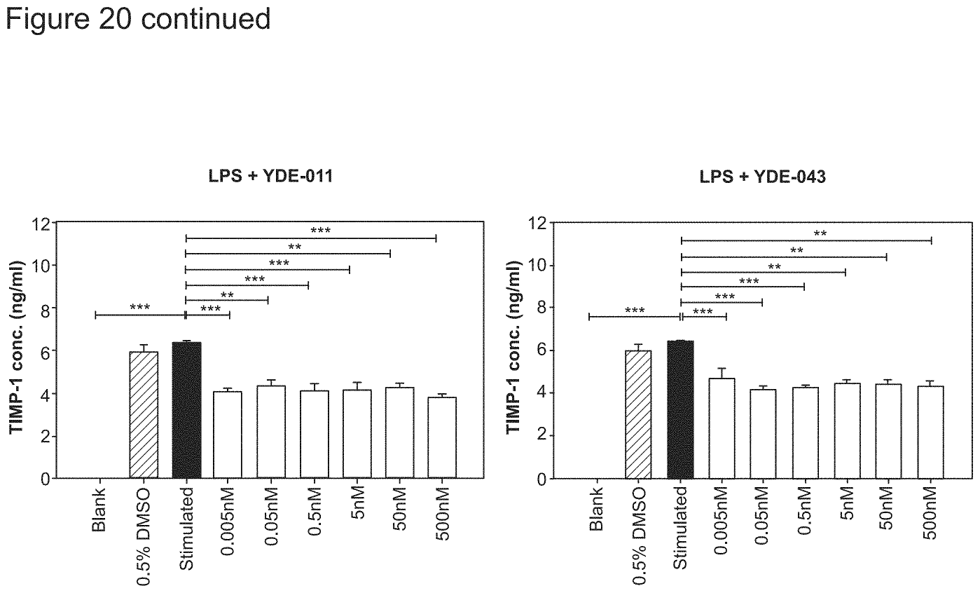

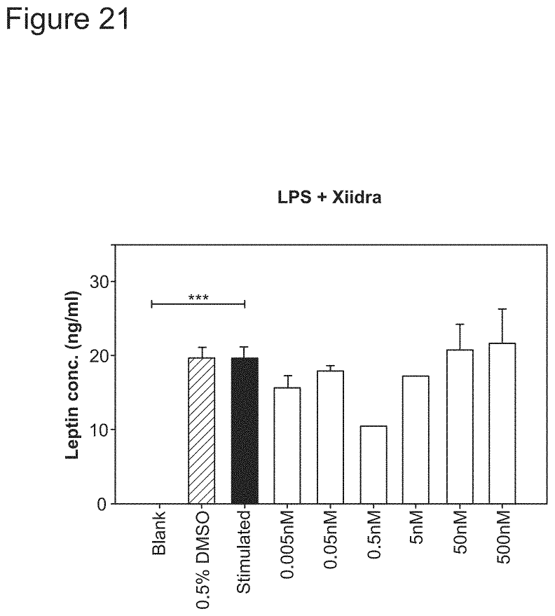

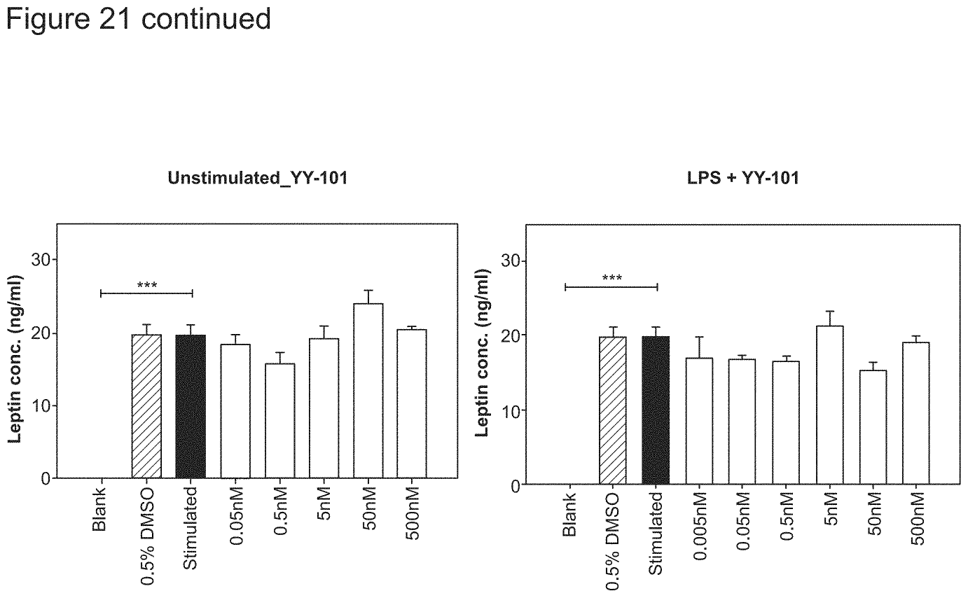

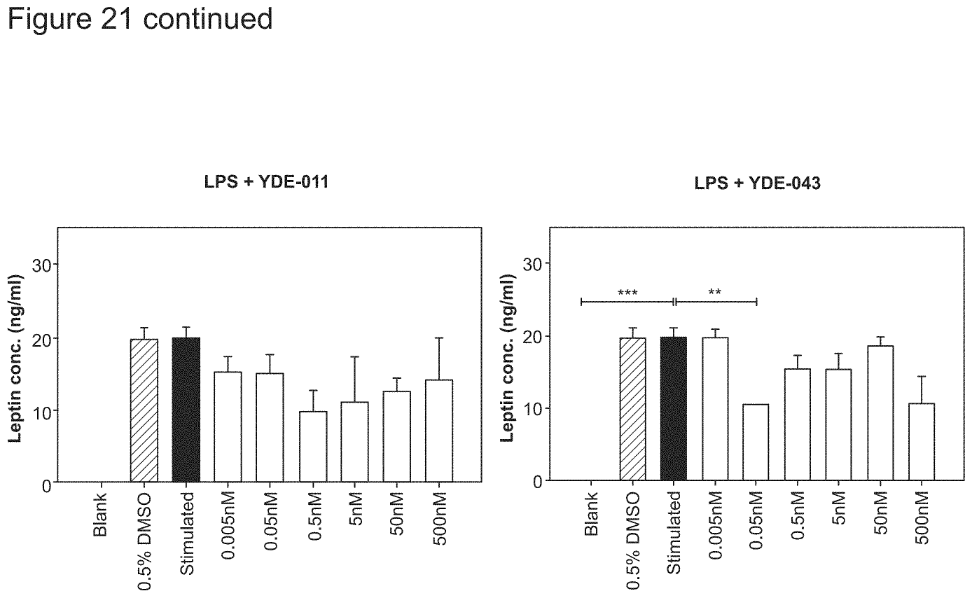

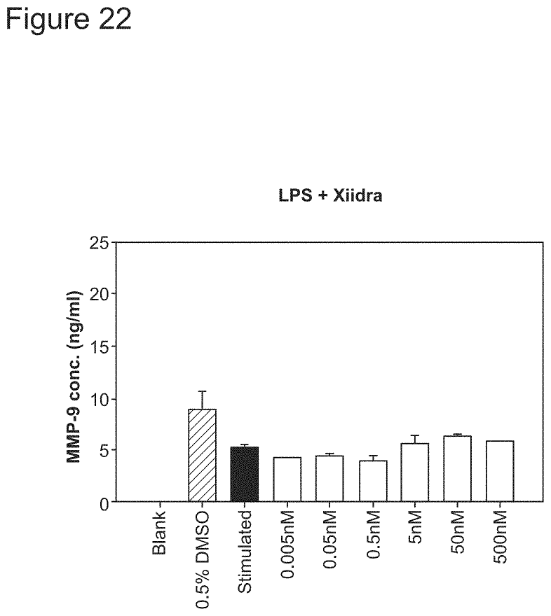

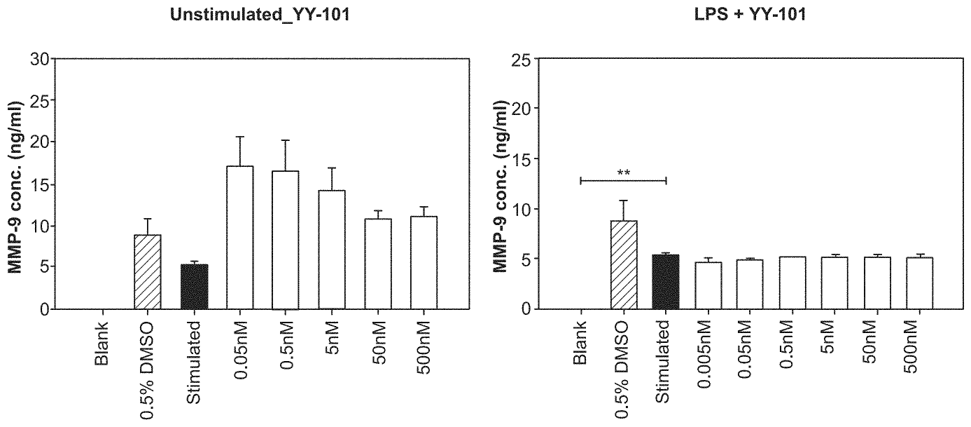

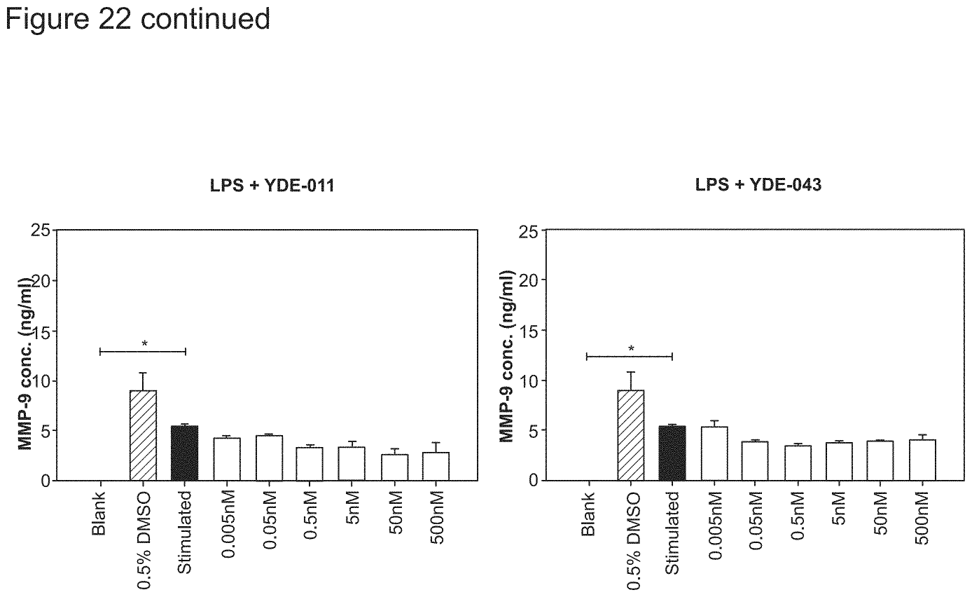

Results are shown in FIGS. 6-22. Human PBMCs frozen purchased from ATCC in a cryopreservative were thawed, washed with Hank's Balanced Salt Solution containing 10% fetal bovine serum, and seeded onto a 24-well plate at a density of 1×106 cells/well with culture RPMI media containing 10% fetal bovine serum in growth media in a 24 well plate for 24 hrs and maintained at 37° C. in an atmosphere of 95% air and 5% CO2. To establish, a pilot study was conducted to establish the timeline and pro-inflammatory stimulant, the cells are treated with stimulant(s), poly(I:C) at 25 ug/mL, LPS at 5 or 25 ug/mL at or PMA (5 ng/ml)/ionomycin (1 ug/ml), supernatants from treated cells are harvested at 12, 24 & 48 h and placed in 96 well for IL-6 ELISA measurement. For the main study, at the end of 24 hr culture, cells are pretreated with YY-101, YDE-011, YDE-043 or reference compound, Xiidra® at various concentrations for 2 h before stimulation. An equal volume of 0.5% DMSO is used as a vehicle control. Then, the cells are treated with LPS. 20 uL of supernatants from treated cells are harvested at 24 hr post-LPS treatment and placed in 96 well for Luminex cytokine & chemokine profiling using a Luminex 200 multiplex assay.

In the pilot studies, all three stimulants (poly I:C, LPS and PMA/Ionomycin) significantly induced IL-6 production in hPBMCs in a time-dependent manner. The reference compound, Xiidra® and YDE-011 significantly reduced IL-6 levels induced by LPS or poly I:C. In the main test, hPBMCs stimulated by LPS at 5 ug/mL for 24 hrs were co-treated with test and reference compounds with various concentrations and from which cytokine release was measured. Among 30 cytokines and chemokines evaluated, YY-101, YDE-011 and YDE-043 at as low as 5 μM significantly reduced LPS-induced pro-inflammatory cytokines and chemokines, IL-2, IL-6, IL-8, IL-10, TNF-α, MMP3, CCL-2, CCL-3 and CCL-4. Among the compounds, YDE-043 significantly lowered LPS-induced IL-1a and IL-1b levels. In contrast, Xiidra® up to 500 nM didn't affect any of LPS-induced cytokine/chemokine levels. Interestingly, the compound YY-101 didn't affect the basal levels of cytokine production.

### IL-6 ELISA in 1^{st} Pilot Test:

IL-6 was measured by sandwich ELISA assay. poly I:C, LPS and PMA/Ionomycin significantly induced IL-6 production in hPBMCs in a time-dependent manner. Among all, LPS induced the highest level of IL-6 at 5 and 25 ug/mL equally.

In the presence of Xiidra® at 30 μM, the reference compound, IL-6 induction was moderately reduced in all stimulants at 24 and 48 hr. Based on strong IL-6 stimulation induced by LPS and poly I:C at 24 and 48 hrs and moderate effects of Xiidra® in the first pilot study, the second pilot study was designed to repeat the stimulants' effect and to elucidate the compound, YDE-011's modulatory effects.

### IL-6 ELISA in 2^{nd} Pilot Test.

Similar to the first pilot study, a similar level of IL-6 induction was observed when hPBMCs were stimulated by poly I:C, LPS at 5 ug/mL or PMA/Ionomycin. YDE-011 at 1 uM effectively and significantly reduced IL-6 induction in PBMCs stimulated by poly I:C, LPS or PMA/Ionomycin. The positive modulatory effect of YDE-011 was diminished at a higher concentration.

### Multi-Cytokine Assessment in PBMCs Stimulated by LPS.

In the main test, hPBMCs were stimulated by LPS at 5 ug/mL for 24 hrs and co-treated with vehicle (0.5% DMSO), test articles and reference compound, Xiidra® at various concentrations (500 nM to 5 μM). After 24 hr treatment, aliquots of hPBMC media were extracted, diluted in 1:200, and measured for cytokine levels using Luminex multiplex system.

Among 30 cytokines and chemokines evaluated, levels of 14 cytokines/chemokines passed the detection QC and the levels of 16 cytokines below the detection levels.

### Conclusions

LPS effectively induced various pro-inflammatory cytokines in human PBMCs and its induction was potently and significantly reduced by all the compounds. Among those compounds, YDE-043 more effectively lowered cytokine and chemokine production. The reference marketed compound, Xiidra® at similar concentrations didn't modulate any of cytokine/chemokine levels, suggesting that YY and YDE compounds are more potent and effective immunomodulators in lowering pro-inflammatory cytokine/chemokine productions in human PBMCs when stimulated by endotoxins, such as LPS.

In conclusion, all of the test compounds, YY-101, YDE-011, YDE-043, potently and significantly reduced pro-inflammatory cytokine/chemokine production in human PBMCs stimulated by LPS.

### Annex 1: 1^{st} Pilot Study Raw Data

TABLE 7

| Sample | | 12 hr | 24 hr | 48 hr |
|---|---|---|---|---|
| PBMC + 0.5% DMSO | 1st | 0 | 0 | 0 |
| | 2nd | 0 | 0 | 0 |
| | 3rd | 0 | 0 | 0 |
| | Mean | 0 | 0 | 0 |
| | STDER | 0 | 0 | 0 |
| PBMC + Poly I:C(25 ug/ml) | 1st | 15338.9 | 20950.9 | 26257.3 |
| | 2nd | 11022.2 | 17695.4 | 21275.3 |
| | 3rd | 11068.1 | 18514.6 | 19606 |
| | Mean | 12476.4 | 19053.6 | 22379.5 |
| | STDER | 1431.3 | 977.7 | 1997.9 |
| PBMC + Poly I:C(25 ug/ml) + | 1st | 16701.1 | 14320.1 | 18990.1 |
| Xiidra ® 10 uM | 2nd | 15193.1 | 15773 | 19515.5 |
| | 3rd | 14460 | 16445.9 | 23645 |
| | Mean | 15451.4 | 15513 | 20716.8 |
| | STDER | 659.7 | 627.3 | 1471.9 |
| PBMC + LPS(5 ug/ml) | 1st | 14335.4 | 23909.6 | 33897.3 |
| | 2nd | 18694.5 | 22492.3 | 31019.9 |
| | 3rd | 17440.3 | 21943.3 | 30259.1 |
| | Mean | 16823.4 | 22781.7 | 31725.4 |
| | STDER | 1295.6 | 585.8 | 1107.9 |
| PBMC + LPS(5 ug/ml) + | 1st | 14546.1 | 17436.8 | 23737.8 |
| Xiidra ® 10 uM | 2nd | 14465.7 | 18053 | 24780.7 |
| | 3rd | 13637.7 | 17850.2 | 27375.7 |
| | Mean | 14216.5 | 17780 | 25298.1 |
| | STDER | 290.3 | 181.3 | 1081.6 |
| PBMC + LPS(25 ug/ml) | 1st | 16538 | 21652.8 | 32026.4 |
| | 2nd | 16547.8 | 20628.4 | 28839.7 |
| | 3rd | 15060.5 | 21084.2 | 31748.5 |
| | Mean | 16048.8 | 21121.8 | 30871.5 |
| | STDER | 494.2 | 296.3 | 1019.1 |
| PBMC + LPS(25 ug/ml) + | 1st | 12065 | 18166.2 | 24395.9 |
| Xiidra ® 10 uM | 2nd | 20113.3 | 16796.5 | 24754.1 |
| | 3rd | 17779.6 | 17509.3 | 29892.3 |
| | Mean | 16652.7 | 17490.7 | 26347.5 |
| | STDER | 2390.7 | 395.5 | 1775.5 |
| PBMC + PMA(5 | 1st | 1265.6 | 5302.9 | 12535.4 |
| ng/ml)/Ionomycin(0.5 ug/ml) | 2nd | 1186.5 | 4731.7 | 12151.6 |
| | 3rd | 1500.8 | 4977.8 | 13166.8 |
| | Mean | 1317.6 | 5004.1 | 12617.9 |
| | STDER | 94.4 | 165.4 | 296 |
| PBMC + PMA(5 | 1st | 1150.9 | 3528 | 7815.5 |
| ng/ml)/Ionomycin(0.5 ug/ml) + | 2nd | 1196.5 | 3455.6 | 8506.2 |
| Xiidra ® 10 uM | 3rd | 1096.5 | 3622.9 | 9128.3 |
| | Mean | 1148 | 3535.5 | 8483.3 |
| | STDER | 28.9 | 48.5 | 379.1 |

### Annex 2: 2^{nd} Pilot Study Raw Data

TABLE 8

| | |
|---|---|
| Sample | 24 hr |

| | | |
|---|---|---|
| PBMC + 0.5% DMSO | 1st | 0 |
| | 2nd | 0 |
| | Mean | 0 |
| | STDER | 0 |
| PBMC + Poly I:C(25 ug/ml) | 1st | 24239.8 |
| | 2nd | 18619.3 |
| | Mean | 21429.5 |
| | STDER | 2810.2 |
| PBMC + Poly I:C(25 ug/ml) + | 1st | 39230.7 |
| YDE-011 30 uM | 2nd | 30634.7 |
| | Mean | 34932.7 |
| | STDER | 4298 |
| PBMC + Poly I:C(25 ug/ml) + | 1st | 13701 |
| YDE-011 1 uM | 2nd | 12585.1 |
| | Mean | 13143.1 |
| | STDER | 557.9 |
| PBMC + LPS(5 ug/ml) | 1st | 29610.6 |
| | 2nd | 22576 |
| | Mean | 26093.3 |
| | STDER | 3517.3 |
| PBMC + LPS(5 ug/ml) + | 1st | 23199.5 |
| YDE-011 30 uM | 2nd | 18310.8 |
| | Mean | 20755.2 |
| | STDER | 2444.3 |
| PBMC + LPS(5 ug/ml) + | 1st | 14532.1 |
| YDE-011 1 uM | 2nd | 12561.2 |
| | Mean | 13546.7 |
| | STDER | 985.4 |
| PBMC + PMA(1 | 1st | 3031.7 |
| ng/ml)/Ionomycin(0.5 ug/ml) | 2nd | 5119.3 |
| | Mean | 4075.5 |
| | STDER | 1043.8 |
| PBMC + PMA(1 | 1st | 3114.6 |
| ng/ml)/Ionomycin(0.5 ug/ml + | 2nd | 3025 |
| YDE-011 30 uM | Mean | 3069.8 |
| | STDER | 44.8 |
| PBMC + PMA(1 | 1st | 2817.2 |
| ng/ml)/Ionomycin(0.5 ug/ml + | 2nd | 3828.2 |
| YDE-011 1 uM | Mean | 3322.7 |
| | STDER | 505.5 |

### Annex 3. The Main Study Raw Data

TABLE 9
Bead name: IL-2

| | | | | | Concentration |
|---|---|---|---|---|---|
| Sample | | MFI | Count | Dilution | (pg/ml) |
| Blank | 1st | 29.0 | 67.0 | 1.0 | <0.45 |
| | 2nd | 31.0 | 67.0 | 1.0 | 0.45 |
| | Mean | 30.0 | 67.0 | 1.0 | #DIV/0! |
| | STDER | 1.0 | 0.0 | 0.0 | #DIV/0! |
| PBMC + 0.5% DMSO | 1st | 30.0 | 87.0 | 200.0 | 5.9 |
| | 2nd | 30.0 | 86.0 | 200.0 | 5.9 |
| | 3rd | 32.0 | 107.0 | 200.0 | 173.5 |
| | Mean | 30.7 | 93.3 | 200.0 | 61.8 |
| | STDER | 0.7 | 6.8 | 0.0 | 55.9 |
| PBMC + LPS(5 ug/ml) | 1st | 88.5 | 68.0 | 200.0 | 4607.6 |
| | 2nd | 74.0 | 85.0 | 200.0 | 3485.4 |
| | 3rd | 79.0 | 107.0 | 200.0 | 3873.0 |
| | Mean | 80.5 | 86.7 | 200.0 | 3988.7 |
| | STDER | 4.3 | 11.3 | 0.0 | 329.1 |
| PBMC + YY101 500 nM | 1st | 31.5 | 86.0 | 200.0 | 132.3 |
| | 2nd | 33.0 | 102.0 | 200.0 | 255.5 |
| | Mean | 32.3 | 94.0 | 200.0 | 193.9 |
| | STDER | 0.8 | 8.0 | 0.0 | 61.6 |
| PBMC + YY101 50 nM | 1st | 30.5 | 88.0 | 200.0 | 48.8 |
| | 2nd | 32.0 | 75.0 | 200.0 | 173.5 |
| | Mean | 31.3 | 81.5 | 200.0 | 111.2 |
| | STDER | 0.8 | 6.5 | 0.0 | 62.4 |
| PBMC + YY101 5 nM | 1st | 32.0 | 86.0 | 200.0 | 173.5 |
| | 2nd | 32.0 | 101.0 | 200.0 | 173.5 |
| | Mean | 32.0 | 93.5 | 200.0 | 173.5 |
| | STDER | 0.0 | 7.5 | 0.0 | 0.0 |
| PBMC + YY101 0.5 nM | 1st | 32.0 | 84.0 | 200.0 | 173.5 |
| | 2nd | 34.0 | 92.0 | 200.0 | 337.0 |
| | Mean | 33.0 | 88.0 | 200.0 | 255.2 |
| | STDER | 1.0 | 4.0 | 0.0 | 81.7 |
| PBMC + YY101 0.05 uM | 1st | 38.0 | 105.0 | 200.0 | 659.4 |
| | 2nd | 34.0 | 92.0 | 200.0 | 337.0 |
| | Mean | 36.0 | 98.5 | 200.0 | 498.2 |
| | STDER | 2.0 | 6.5 | 0.0 | 161.2 |
| PBMC + LPS(5 ug/ml) + | 1st | 88.0 | 101.0 | 200.0 | 4569.0 |
| Xiidra 500 nM | 2nd | 86.0 | 97.0 | 200.0 | 4414.5 |
| | Mean | 87.0 | 99.0 | 200.0 | 4491.8 |
| | STDER | 1.0 | 2.0 | 0.0 | 77.3 |
| PBMC + LPS(5 ug/ml) + | 1st | 90.0 | 69.0 | 200.0 | 4723.4 |
| Xiidra 50 nM | 2nd | 93.0 | 95.0 | 200.0 | 4954.9 |
| | Mean | 91.5 | 82.0 | 200.0 | 4839.1 |
| | STDER | 1.5 | 13.0 | 0.0 | 115.7 |
| PBMC + LPS(5 ug/ml) + | 1st | 83.0 | 70.0 | 200.0 | 4182.6 |
| Xiidra 5 nM | 2nd | 95.0 | 79.0 | 200.0 | 5109.1 |
| | Mean | 89.0 | 74.5 | 200.0 | 4645.8 |
| | STDER | 6.0 | 4.5 | 0.0 | 463.2 |
| PBMC + LPS(5 ug/ml) + | 1st | 76.0 | 87.0 | 200.0 | 3640.5 |
| Xiidra 0.5 nM | 2nd | 79.5 | 80.0 | 200.0 | 3911.7 |
| | Mean | 77.8 | 83.5 | 200.0 | 3776.1 |
| | STDER | 1.8 | 3.5 | 0.0 | 135.6 |
| PBMC + LPS(5 ug/ml) + | 1st | 74.0 | 103.0 | 200.0 | 3485.4 |
| Xiidra 0.05 nM | 2nd | 81.5 | 94.0 | 200.0 | 4066.6 |
| | Mean | 77.8 | 98.5 | 200.0 | 3776.0 |
| | STDER | 3.8 | 4.5 | 0.0 | 290.6 |
| PBMC + LPS(5 ug/ml) + | 1st | 79.0 | 74.0 | 200.0 | 3873.0 |
| Xiidra 0.005 nM | 2nd | 72.0 | 67.0 | 200.0 | 3330.1 |
| | Mean | 75.5 | 70.5 | 200.0 | 3601.6 |
| | STDER | 3.5 | 3.5 | 0.0 | 271.4 |
| PBMC + LPS(5 ug/ml) + | 1st | 64.0 | 68.0 | 200.0 | 2707.7 |
| YY101 500 nM | 2nd | 66.0 | 84.0 | 200.0 | 2863.5 |
| | 3rd | 62.0 | 84.0 | 200.0 | 2551.7 |
| | Mean | 64.0 | 78.7 | 200.0 | 2707.6 |
| | STDER | 1.2 | 5.3 | 0.0 | 90.0 |
| PBMC + LPS(5 ug/ml) + | 1st | 61.0 | 79.0 | 200.0 | 2473.6 |
| YY101 50 nM | 2nd | 64.0 | 96.0 | 200.0 | 2707.7 |
| | 3rd | 66.0 | 99.0 | 200.0 | 2863.5 |
| | Mean | 63.7 | 91.3 | 200.0 | 2681.6 |
| | STDER | 1.5 | 6.2 | 0.0 | 113.3 |
| PBMC + LPS(5 ug/ml) + | 1st | 61.0 | 87.0 | 200.0 | 2473.6 |
| YY101 5 nM | 2nd | 63.0 | 83.0 | 200.0 | 2629.7 |
| | 3rd | 67.0 | 67.0 | 200.0 | 2941.4 |
| | Mean | 63.7 | 79.0 | 200.0 | 2681.6 |
| | STDER | 1.8 | 6.1 | 0.0 | 137.5 |
| PBMC + LPS(5 ug/ml) + | 1st | 66.0 | 102.0 | 200.0 | 2863.5 |
| YY101 0.5 nM | 2nd | 65.0 | 87.0 | 200.0 | 2785.6 |
| | 3rd | 65.5 | 74.0 | 200.0 | 2824.6 |
| | Mean | 65.5 | 87.7 | 200.0 | 2824.6 |
| | STDER | 0.3 | 8.1 | 0.0 | 22.5 |
| PBMC + LPS(5 ug/ml) + | 1st | 65.0 | 82.0 | 200.0 | 2785.6 |
| YY101 0.05 nM | 2nd | 70.0 | 87.0 | 200.0 | 3174.7 |
| | 3rd | 68.5 | 96.0 | 200.0 | 3058.1 |
| | Mean | 67.8 | 88.3 | 200.0 | 3006.1 |
| | STDER | 1.5 | 4.1 | 0.0 | 115.3 |
| PBMC + LPS(5 ug/ml) + | 1st | 64.5 | 76.0 | 200.0 | 2746.7 |
| YY101 0.005 nM | 2nd | 64.0 | 61.0 | 200.0 | 2707.7 |
| | 3rd | 62.0 | 68.0 | 200.0 | 2551.7 |
| | Mean | 63.5 | 68.3 | 200.0 | 2668.7 |
| | STDER | 0.8 | 4.3 | 0.0 | 59.6 |
| PBMC + LPS(5 ug/ml) + | 1st | 67.0 | 85.0 | 200.0 | 2941.4 |
| YDE011 500 nM | 2nd | 37.0 | 95.0 | 200.0 | 579.2 |
| | 3rd | 51.5 | 86.0 | 200.0 | 1729.4 |
| | Mean | 51.8 | 88.7 | 200.0 | 1750.0 |
| | STDER | 8.7 | 3.2 | 0.0 | 682.0 |
| PBMC + LPS(5 ug/ml) + | 1st | 56.0 | 111.0 | 200.0 | 2082.5 |
| YDE011 50 nM | 2nd | 57.0 | 71.0 | 200.0 | 2160.8 |
| | 3rd | 38.0 | 81.0 | 200.0 | 659.4 |
| | Mean | 50.3 | 87.7 | 200.0 | 1634.3 |
| | STDER | 6.2 | 12.0 | 0.0 | 487.9 |
| PBMC + LPS(5 ug/ml) + | 1st | 49.0 | 80.0 | 200.0 | 1532.6 |
| YDE011 5 nM | 2nd | 50.0 | 83.0 | 200.0 | 1611.3 |
| | 3rd | 62.0 | 100.0 | 200.0 | 2551.7 |
| | Mean | 53.7 | 87.7 | 200.0 | 1898.5 |
| | STDER | 4.2 | 6.2 | 0.0 | 327.4 |
| PBMC + LPS(5 ug/ml) + | 1st | 58.0 | 85.0 | 200.0 | 2239.1 |
| YDE011 0.5 nM | 2nd | 54.0 | 82.0 | 200.0 | 1925.7 |
| | 3rd | 57.5 | 92.0 | 200.0 | 2200.0 |
| | Mean | 56.5 | 86.3 | 200.0 | 2121.6 |
| | STDER | 1.3 | 3.0 | 0.0 | 98.6 |
| PBMC + LPS(5 ug/ml) + | 1st | 64.0 | 90.0 | 200.0 | 2707.7 |
| YDE011 0.05 nM | 2nd | 61.0 | 86.0 | 200.0 | 2473.6 |
| | 3rd | 65.5 | 102.0 | 200.0 | 2824.6 |
| | Mean | 63.5 | 92.7 | 200.0 | 2668.6 |
| | STDER | 1.3 | 4.8 | 0.0 | 103.2 |
| PBMC + LPS(5 ug/ml) + | 1st | 58.0 | 79.0 | 200.0 | 2239.1 |
| YDE011 0.005 nM | 2nd | 56.0 | 78.0 | 200.0 | 2082.5 |
| | 3rd | 64.0 | 100.0 | 200.0 | 2707.7 |
| | Mean | 59.3 | 85.7 | 200.0 | 2343.1 |
| | STDER | 2.4 | 7.2 | 0.0 | 187.8 |
| PBMC + LPS(5 ug/ml) + | 1st | 55.0 | 95.0 | 200.0 | 2004.2 |
| YDE043 500 nM | 2nd | 56.0 | 84.0 | 200.0 | 2082.5 |
| | 3rd | 58.0 | 87.0 | 200.0 | 2239.1 |
| | Mean | 56.3 | 88.7 | 200.0 | 2108.6 |
| | STDER | 0.9 | 3.3 | 0.0 | 69.1 |
| PBMC + LPS(5 ug/ml) + | 1st | 62.0 | 90.0 | 200.0 | 2551.7 |
| YDE043 50 nM | 2nd | 60.0 | 102.0 | 200.0 | 2395.5 |
| | 3rd | 58.0 | 87.0 | 200.0 | 2239.1 |
| | Mean | 60.0 | 93.0 | 200.0 | 2395.4 |
| | STDER | 1.2 | 4.6 | 0.0 | 90.2 |
| PBMC + LPS(5 ug/ml) + | 1st | 65.0 | 76.0 | 200.0 | 2785.6 |
| YDE043 5 nM | 2nd | 60.0 | 77.0 | 200.0 | 2395.5 |
| | 3rd | 61.0 | 103.0 | 200.0 | 2473.6 |
| | Mean | 62.0 | 85.3 | 200.0 | 2551.6 |
| | STDER | 1.5 | 8.8 | 0.0 | 119.2 |
| PBMC + LPS(5 ug/ml) + | 1st | 56.0 | 83.0 | 200.0 | 2082.5 |
| YDE043 0.5 nM | 2nd | 59.0 | 98.0 | 200.0 | 2317.3 |
| | 3rd | 56.5 | 92.0 | 200.0 | 2121.7 |
| | Mean | 57.2 | 91.0 | 200.0 | 2173.8 |
| | STDER | 0.9 | 4.4 | 0.0 | 72.6 |
| PBMC + LPS(5 ug/ml) + | 1st | 55.0 | 72.0 | 200.0 | 2004.2 |
| YDE043 0.05 nM | 2nd | 58.0 | 80.0 | 200.0 | 2239.1 |
| | 3rd | 58.0 | 88.0 | 200.0 | 2239.1 |
| | Mean | 57.0 | 80.0 | 200.0 | 2160.8 |
| | STDER | 1.0 | 4.6 | 0.0 | 78.3 |
| PBMC + LPS(5 ug/ml) + | 1st | 58.0 | 82.0 | 200.0 | 2239.1 |
| YDE043 0.005 nM | 2nd | 59.0 | 85.0 | 200.0 | 2317.3 |
| | 3rd | 68.5 | 66.0 | 200.0 | 3058.1 |
| | Mean | 61.8 | 77.7 | 200.0 | 2538.2 |
| | STDER | 3.3 | 5.9 | 0.0 | 260.9 |

TABLE 10
Bead name: IL-6

| | | | | | Concentration |
|---|---|---|---|---|---|
| Sample | | MFI | Count | Dilution | (pg/ml) |
| Blank | 1st | 20.5 | 78.0 | 1.0 | <2.65 |
| | 2nd | 20.0 | 108.0 | 1.0 | <2.65 |
| | Mean | 20.3 | 93.0 | 1.0 | #DIV/0! |
| | STDER | 0.3 | 15.0 | 0.0 | #DIV/0! |
| PBMC + 0.5% DMSO | 1st | 22.5 | 94.0 | 200.0 | 16.1 |
| | 2nd | 22.0 | 93.0 | 200.0 | 5.0 |
| | 3rd | 22.0 | 111.0 | 200.0 | 5.0 |
| | Mean | 22.2 | 99.3 | 200.0 | 8.7 |
| | STDER | 0.2 | 5.8 | 0.0 | 3.7 |
| PBMC + LPS(5 ug/ml) | 1st | 1407.0 | 93.0 | 200.0 | 32871.0 |
| | 2nd | 1182.0 | 74.0 | 200.0 | 27467.5 |
| | 3rd | 1126.5 | 114.0 | 200.0 | 26138.3 |
| | Mean | 1238.5 | 93.7 | 200.0 | 28825.6 |
| | STDER | 85.8 | 11.6 | 0.0 | 2058.8 |
| PBMC + YY101 500 nM | 1st | 21.0 | 114.0 | 200.0 | <2.65 |
| | 2nd | 21.0 | 122.0 | 200.0 | <2.65 |
| | Mean | 21.0 | 118.0 | 200.0 | #DIV/0! |
| | STDER | 0.0 | 4.0 | 0.0 | #DIV/0! |
| PBMC + YY101 50 nM | 1st | 22.0 | 84.0 | 200.0 | 5.0 |
| | 2nd | 20.0 | 106.0 | 200.0 | <2.65 |
| | Mean | 21.0 | 95.0 | 200.0 | 5.0 |
| | STDER | 1.0 | 11.0 | 0.0 | #DIV/0! |
| PBMC + YY101 5 nM | 1st | 22.0 | 103.0 | 200.0 | 5.0 |
| | 2nd | 22.0 | 100.0 | 200.0 | 5.0 |
| | Mean | 22.0 | 101.5 | 200.0 | 5.0 |
| | STDER | 0.0 | 1.5 | 0.0 | 0.0 |
| PBMC + YY101 0.5 nM | 1st | 22.0 | 99.0 | 200.0 | 5.0 |
| | 2nd | 23.0 | 101.0 | 200.0 | 27.3 |
| | Mean | 22.5 | 100.0 | 200.0 | 16.2 |
| | STDER | 0.5 | 1.0 | 0.0 | 11.1 |
| PBMC + YY101 0.05 uM | 1st | 28.0 | 80.0 | 200.0 | 140.1 |
| | 2nd | 20.0 | 106.0 | 200.0 | <2.65 |
| | Mean | 24.0 | 93.0 | 200.0 | 140.1 |
| | STDER | 4.0 | 13.0 | 0.0 | #DIV/0! |
| PBMC + LPS(5 ug/ml) + | 1st | 1422.0 | 94.0 | 200.0 | 33232.1 |
| Xiidra 500 nM | 2nd | 1420.0 | 84.0 | 200.0 | 33183.9 |
| | Mean | 1421.0 | 89.0 | 200.0 | 33208.0 |
| | STDER | 1.0 | 5.0 | 0.0 | 24.1 |
| PBMC + LPS(5 ug/ml) + | 1st | 1365.0 | 97.0 | 200.0 | 31860.4 |
| Xiidra 50 nM | 2nd | 1427.5 | 122.0 | 200.0 | 33364.6 |
| | Mean | 1396.3 | 109.5 | 200.0 | 32612.5 |
| | STDER | 31.3 | 12.5 | 0.0 | 752.1 |
| PBMC + LPS(5 ug/ml) + | 1st | 1234.5 | 114.0 | 200.0 | 28726.1 |
| Xiidra 5 nM | 2nd | 1306.0 | 109.0 | 200.0 | 30442.3 |
| | Mean | 1270.3 | 111.5 | 200.0 | 29584.2 |
| | STDER | 35.8 | 2.5 | 0.0 | 858.1 |
| PBMC + LPS(5 ug/ml) + | 1st | 1094.5 | 108.0 | 200.0 | 25372.4 |
| Xiidra 0.5 nM | 2nd | 1188.5 | 102.0 | 200.0 | 27623.3 |
| | Mean | 1141.5 | 105.0 | 200.0 | 26497.8 |
| | STDER | 47.0 | 3.0 | 0.0 | 1125.4 |
| PBMC + LPS(5 ug/ml) + | 1st | 1112.0 | 127.0 | 200.0 | 25791.2 |
| Xiidra 0.05 nM | 2nd | 1129.0 | 104.0 | 200.0 | 26198.1 |
| | Mean | 1120.5 | 115.5 | 200.0 | 25994.6 |
| | STDER | 8.5 | 11.5 | 0.0 | 203.5 |
| PBMC + LPS(5 ug/ml) + | 1st | 1131.5 | 108.0 | 200.0 | 26257.9 |
| Xiidra 0.005 nM | 2nd | 1091.0 | 94.0 | 200.0 | 25288.7 |
| | Mean | 1111.3 | 101.0 | 200.0 | 25773.3 |
| | STDER | 20.3 | 7.0 | 0.0 | 484.6 |
| PBMC + LPS(5 ug/ml) + | 1st | 749.0 | 65.0 | 200.0 | 17129.3 |
| YY101 500 nM | 2nd | 775.0 | 118.0 | 200.0 | 17748.1 |
| | 3rd | 727.5 | 84.0 | 200.0 | 16617.9 |
| | Mean | 750.5 | 89.0 | 200.0 | 17165.1 |
| | STDER | 13.7 | 15.5 | 0.0 | 326.8 |
| PBMC + LPS(5 ug/ml) + | 1st | 715.0 | 87.0 | 200.0 | 16320.6 |
| YY101 50 nM | 2nd | 730.5 | 112.0 | 200.0 | 16689.2 |
| | 3rd | 780.5 | 98.0 | 200.0 | 17879.0 |
| | Mean | 742.0 | 99.0 | 200.0 | 16962.9 |
| | STDER | 19.8 | 7.2 | 0.0 | 470.2 |
| PBMC + LPS(5 ug/ml) + | 1st | 727.0 | 95.0 | 200.0 | 16606.0 |
| YY101 5 nM | 2nd | 720.0 | 84.0 | 200.0 | 16439.5 |
| | 3rd | 813.0 | 105.0 | 200.0 | 18652.8 |
| | Mean | 753.3 | 94.7 | 200.0 | 17232.8 |
| | STDER | 29.9 | 6.1 | 0.0 | 711.7 |
| PBMC + LPS(5 ug/ml) + | 1st | 788.5 | 94.0 | 200.0 | 18069.4 |
| YY101 0.5 nM | 2nd | 754.0 | 98.0 | 200.0 | 17248.3 |
| | 3rd | 816.5 | 96.0 | 200.0 | 18736.2 |
| | Mean | 786.3 | 96.0 | 200.0 | 18018.0 |
| | STDER | 18.1 | 1.2 | 0.0 | 430.3 |
| PBMC + LPS(5 ug/ml) + | 1st | 769.0 | 94.0 | 200.0 | 17605.3 |
| YY101 0.05 nM | 2nd | 832.0 | 99.0 | 200.0 | 19105.4 |
| | 3rd | 803.0 | 87.0 | 200.0 | 18414.7 |
| | Mean | 801.3 | 93.3 | 200.0 | 18375.1 |
| | STDER | 18.2 | 3.5 | 0.0 | 433.5 |
| PBMC + LPS(5 ug/ml) + | 1st | 817.5 | 106.0 | 200.0 | 18760.0 |
| YY101 0.005 nM | 2nd | 772.0 | 85.0 | 200.0 | 17676.7 |
| | 3rd | 775.0 | 97.0 | 200.0 | 17748.1 |
| | Mean | 788.2 | 96.0 | 200.0 | 18061.6 |
| | STDER | 14.7 | 6.1 | 0.0 | 349.8 |
| PBMC + LPS(5 ug/ml) + | 1st | 843.0 | 88.0 | 200.0 | 19367.5 |
| YDE011 500 nM | 2nd | 422.5 | 96.0 | 200.0 | 9383.5 |
| | 3rd | 657.5 | 98.0 | 200.0 | 14953.9 |
| | Mean | 641.0 | 94.0 | 200.0 | 14568.3 |
| | STDER | 121.7 | 3.1 | 0.0 | 2888.6 |
| PBMC + LPS(5 ug/ml) + | 1st | 732.0 | 132.0 | 200.0 | 16724.9 |
| YDE011 50 nM | 2nd | 754.0 | 97.0 | 200.0 | 17248.3 |
| | 3rd | 444.0 | 97.0 | 200.0 | 9892.0 |
| | Mean | 643.3 | 108.7 | 200.0 | 14621.7 |
| | STDER | 99.9 | 11.7 | 0.0 | 2369.7 |
| PBMC + LPS(5 ug/ml) + | 1st | 537.0 | 93.0 | 200.0 | 12094.3 |
| YDE011 5 nM | 2nd | 573.0 | 105.0 | 200.0 | 12947.9 |
| | 3rd | 684.0 | 93.0 | 200.0 | 15583.6 |
| | Mean | 598.0 | 97.0 | 200.0 | 13541.9 |
| | STDER | 44.2 | 4.0 | 0.0 | 1050.1 |
| PBMC + LPS(5 ug/ml) + | 1st | 673.0 | 99.0 | 200.0 | 15322.2 |
| YDE011 0.5 nM | 2nd | 687.0 | 97.0 | 200.0 | 15654.9 |
| | 3rd | 676.0 | 104.0 | 200.0 | 15393.5 |
| | Mean | 678.7 | 100.0 | 200.0 | 15456.8 |
| | STDER | 4.3 | 2.1 | 0.0 | 101.1 |
| PBMC + LPS(5 ug/ml) + | 1st | 821.0 | 96.0 | 200.0 | 18843.4 |
| YDE011 0.05 nM | 2nd | 800.0 | 81.0 | 200.0 | 18343.3 |
| | 3rd | 810.0 | 84.0 | 200.0 | 18581.4 |
| | Mean | 810.3 | 87.0 | 200.0 | 18589.3 |
| | STDER | 6.1 | 4.6 | 0.0 | 144.4 |
| PBMC + LPS(5 ug/ml) + | 1st | 737.0 | 73.0 | 200.0 | 16843.8 |
| YDE011 0.005 nM | 2nd | 670.0 | 89.0 | 200.0 | 15250.9 |
| | 3rd | 712.0 | 114.0 | 200.0 | 16249.2 |
| | Mean | 706.3 | 92.0 | 200.0 | 16114.6 |
| | STDER | 19.5 | 11.9 | 0.0 | 464.7 |
| PBMC + LPS(5 ug/ml) + | 1st | 612.0 | 95.0 | 200.0 | 13873.4 |
| YDE043 500 nM | 2nd | 612.0 | 95.0 | 200.0 | 13873.4 |
| | 3rd | 654.5 | 86.0 | 200.0 | 14882.6 |
| | Mean | 626.2 | 92.0 | 200.0 | 14209.8 |
| | STDER | 14.2 | 3.0 | 0.0 | 336.4 |
| PBMC + LPS(5 ug/ml) + | 1st | 742.0 | 89.0 | 200.0 | 16962.8 |
| YDE043 50 nM | 2nd | 725.5 | 94.0 | 200.0 | 16570.3 |
| | 3rd | 645.5 | 118.0 | 200.0 | 14668.8 |
| | Mean | 704.3 | 100.3 | 200.0 | 16067.3 |
| | STDER | 29.8 | 9.0 | 0.0 | 708.4 |
| PBMC + LPS(5 ug/ml) + | 1st | 717.5 | 80.0 | 200.0 | 16380.0 |
| YDE043 5 nM | 2nd | 753.0 | 73.0 | 200.0 | 17224.5 |
| | 3rd | 698.0 | 121.0 | 200.0 | 15916.4 |
| | Mean | 722.8 | 91.3 | 200.0 | 16507.0 |
| | STDER | 16.1 | 15.0 | 0.0 | 382.9 |
| PBMC + LPS(5 ug/ml) + | 1st | 673.0 | 83.0 | 200.0 | 15322.2 |
| YDE043 0.5 nM | 2nd | 663.0 | 101.0 | 200.0 | 15084.6 |
| | 3rd | 618.5 | 92.0 | 200.0 | 14027.7 |
| | Mean | 651.5 | 92.0 | 200.0 | 14811.5 |
| | STDER | 16.8 | 5.2 | 0.0 | 397.9 |
| PBMC + LPS(5 ug/ml) + | 1st | 591.5 | 90.0 | 200.0 | 13386.9 |
| YDE043 0.05 nM | 2nd | 647.5 | 96.0 | 200.0 | 14716.3 |
| | 3rd | 645.0 | 99.0 | 200.0 | 14657.0 |
| | Mean | 628.0 | 95.0 | 200.0 | 14253.4 |
| | STDER | 18.3 | 2.6 | 0.0 | 433.6 |
| PBMC + LPS(5 ug/ml) + | 1st | 690.0 | 89.0 | 200.0 | 15726.2 |
| YDE043 0.005 nM | 2nd | 734.0 | 87.0 | 200.0 | 16772.5 |
| | 3rd | 892.0 | 99.0 | 200.0 | 20535.6 |
| | Mean | 772.0 | 91.7 | 200.0 | 17678.1 |
| | STDER | 61.3 | 3.7 | 0.0 | 1460.3 |

TABLE 11
Bead name: IL-8/CXCL8

| | | | | | Concen- |
|---|---|---|---|---|---|
| | | | | Dilu- | tration |
| Sample | | MFI | Count | tion | (pg/ml) |
| Blank | 1st | 32.0 | 76.0 | 1.0 | 0.3 |
| | 2nd | 30.0 | 90.0 | 1.0 | 0.2 |
| | Mean | 31.0 | 83.0 | 1.0 | 0.3 |
| | STDER | 1.0 | 7.0 | 0.0 | 0.1 |
| PBMC + 0.5% DMSO | 1st | 287.5 | 78.0 | 200.0 | 3955.9 |
| | 2nd | 110.5 | 86.0 | 200.0 | 1155.1 |
| | 3rd | 113.0 | 91.0 | 200.0 | 1192.3 |
| | Mean | 170.3 | 85.0 | 200.0 | 2101.1 |
| | STDER | 58.6 | 3.8 | 0.0 | 927.5 |
| PBMC + LPS(5 ug/ml) | 1st | 4811.0 | 82.0 | 200.0 | 100889.1 |
| | 2nd | 4193.0 | 91.0 | 200.0 | 84243.2 |
| | 3rd | 4083.0 | 83.0 | 200.0 | 81501.2 |
| | Mean | 4362.3 | 85.3 | 200.0 | 88877.9 |
| | STDER | 226.6 | 2.8 | 0.0 | 6057.6 |
| PBMC + YY101 500 nM | 1st | 120.0 | 87.0 | 200.0 | 1297.1 |
| | 2nd | 165.0 | 93.0 | 200.0 | 1986.4 |
| | Mean | 142.5 | 90.0 | 200.0 | 1641.8 |
| | STDER | 22.5 | 3.0 | 0.0 | 344.6 |
| PBMC + YY101 50 nM | 1st | 166.5 | 76.0 | 200.0 | 2009.8 |
| | 2nd | 117.0 | 85.0 | 200.0 | 1252.1 |
| | Mean | 141.8 | 80.5 | 200.0 | 1630.9 |
| | STDER | 24.8 | 4.5 | 0.0 | 378.8 |
| PBMC + YY101 5 nM | 1st | 281.0 | 93.0 | 200.0 | 3848.8 |
| | 2nd | 151.0 | 87.0 | 200.0 | 1769.4 |
| | Mean | 216.0 | 90.0 | 200.0 | 2809.1 |
| | STDER | 65.0 | 3.0 | 0.0 | 1039.7 |
| PBMC + YY101 0.5 nM | 1st | 164.0 | 81.0 | 200.0 | 1970.8 |
| | 2nd | 368.0 | 89.0 | 200.0 | 5300.9 |
| | Mean | 266.0 | 85.0 | 200.0 | 3635.9 |
| | STDER | 102.0 | 4.0 | 0.0 | 1665.0 |
| PBMC + YY101 0.05 | 1st | 836.5 | 74.0 | 200.0 | 13586.7 |
| uM | 2nd | 207.0 | 97.0 | 200.0 | 2648.7 |
| | Mean | 521.8 | 85.5 | 200.0 | 8117.7 |
| | STDER | 314.8 | 11.5 | 0.0 | 5469.0 |
| PBMC + LPS(5 ug/ml) + | 1st | 4811.0 | 90.0 | 200.0 | 100889.1 |
| Xiidra 500 nM | 2nd | 4424.0 | 73.0 | 200.0 | 90193.3 |
| | Mean | 4617.5 | 81.5 | 200.0 | 95541.2 |
| | STDER | 193.5 | 8.5 | 0.0 | 5347.9 |
| PBMC + LPS(5 ug/ml) + | 1st | 4604.0 | 66.0 | 200.0 | 95040.0 |
| Xiidra 50 nM | 2nd | 4553.0 | 99.0 | 200.0 | 93646.0 |
| | Mean | 4578.5 | 82.5 | 200.0 | 94343.0 |
| | STDER | 25.5 | 16.5 | 0.0 | 697.0 |
| PBMC + LPS(5 ug/ml) + | 1st | 4476.0 | 84.0 | 200.0 | 91572.9 |
| Xiidra 5 nM | 2nd | 4697.5 | 74.0 | 200.0 | 97642.3 |
| | Mean | 4586.8 | 79.0 | 200.0 | 94607.6 |
| | STDER | 110.8 | 5.0 | 0.0 | 3034.7 |
| PBMC + LPS(5 ug/ml) + | 1st | 4067.5 | 78.0 | 200.0 | 81119.1 |
| Xiidra 0.5 nM | 2nd | 4450.5 | 96.0 | 200.0 | 90894.4 |
| | Mean | 4259.0 | 87.0 | 200.0 | 86006.8 |
| | STDER | 191.5 | 9.0 | 0.0 | 4887.6 |
| PBMC + LPS(5 ug/ml) + | 1st | 4169.0 | 109.0 | 200.0 | 83640.3 |
| Xiidra 0.05 nM | 2nd | 4413.0 | 73.0 | 200.0 | 89903.5 |
| | Mean | 4291.0 | 91.0 | 200.0 | 86771.9 |
| | STDER | 122.0 | 18.0 | 0.0 | 3131.6 |
| PBMC + LPS(5 ug/ml) + | 1st | 4154.0 | 91.0 | 200.0 | 83264.8 |
| Xiidra 0.005 nM | 2nd | 4103.5 | 100.0 | 200.0 | 82008.2 |
| | Mean | 4128.8 | 95.5 | 200.0 | 82636.5 |
| | STDER | 25.3 | 4.5 | 0.0 | 628.3 |
| PBMC + LPS(5 ug/ml) + | 1st | 3175.0 | 77.0 | 200.0 | 60492.6 |
| YY101 500 nM | 2nd | 3150.0 | 81.0 | 200.0 | 59945.6 |
| | 3rd | 2895.0 | 68.0 | 200.0 | 54438.6 |
| | Mean | 3073.3 | 75.3 | 200.0 | 58292.3 |
| | STDER | 89.5 | 3.8 | 0.0 | 1933.3 |
| PBMC + LPS(5 ug/ml) + | 1st | 2910.0 | 67.0 | 200.0 | 54759.1 |
| YY101 50 nM | 2nd | 2830.5 | 114.0 | 200.0 | 53065.0 |
| | 3rd | 2978.0 | 91.0 | 200.0 | 56217.2 |
| | Mean | 2906.2 | 90.7 | 200.0 | 54680.4 |
| | STDER | 42.6 | 13.6 | 0.0 | 910.8 |
| PBMC + LPS(5 ug/ml) + | 1st | 2948.5 | 96.0 | 200.0 | 55583.6 |
| YY101 5 nM | 2nd | 2967.0 | 77.0 | 200.0 | 55980.8 |
| | 3rd | 3170.0 | 75.0 | 200.0 | 60383.1 |
| | Mean | 3028.5 | 82.7 | 200.0 | 57315.8 |
| | STDER | 71.0 | 6.7 | 0.0 | 1537.9 |
| PBMC + LPS(5 ug/ml) + | 1st | 3194.0 | 89.0 | 200.0 | 60909.2 |
| YY101 0.5 nM | 2nd | 3056.0 | 95.0 | 200.0 | 57900.7 |
| | 3rd | 3241.0 | 61.0 | 200.0 | 61943.2 |
| | Mean | 3163.7 | 81.7 | 200.0 | 60251.0 |
| | STDER | 55.5 | 10.5 | 0.0 | 1212.5 |
| PBMC + LPS(5 ug/ml) + | 1st | 3031.0 | 88.0 | 200.0 | 57359.8 |
| YY101 0.05 nM | 2nd | 3177.0 | 77.0 | 200.0 | 60536.4 |
| | 3rd | 3247.0 | 77.0 | 200.0 | 62075.6 |
| | Mean | 3151.7 | 80.7 | 200.0 | 59990.6 |
| | STDER | 63.6 | 3.7 | 0.0 | 1388.4 |
| PBMC + LPS(5 ug/ml) + | 1st | 3223.0 | 64.0 | 200.0 | 61546.6 |
| YY101 0.005 nM | 2nd | 3009.0 | 78.0 | 200.0 | 56884.9 |
| | 3rd | 3015.0 | 75.0 | 200.0 | 57014.3 |
| | Mean | 3082.3 | 72.3 | 200.0 | 58481.9 |
| | STDER | 70.4 | 4.3 | 0.0 | 1532.8 |
| PBMC + LPS(5 ug/ml) + | 1st | 3436.0 | 70.0 | 200.0 | 66288.2 |
| YDE011 500 nM | 2nd | 1788.0 | 97.0 | 200.0 | 31741.3 |
| | 3rd | 2708.0 | 79.0 | 200.0 | 50476.2 |
| | Mean | 2644.0 | 82.0 | 200.0 | 49501.9 |
| | STDER | 476.8 | 7.9 | 0.0 | 9984.7 |
| PBMC + LPS(5 ug/ml) + | 1st | 2885.5 | 86.0 | 200.0 | 54235.8 |
| YDE011 50 nM | 2nd | 2934.0 | 74.0 | 200.0 | 55272.7 |
| | 3rd | 1763.5 | 68.0 | 200.0 | 31257.8 |
| | Mean | 2527.7 | 76.0 | 200.0 | 46922.1 |
| | STDER | 382.3 | 5.3 | 0.0 | 7837.9 |
| PBMC + LPS(5 ug/ml) + | 1st | 2473.5 | 80.0 | 200.0 | 45587.7 |
| YDE011 5 nM | 2nd | 2353.0 | 76.0 | 200.0 | 43107.6 |
| | 3rd | 2728.5 | 94.0 | 200.0 | 50907.7 |
| | Mean | 2518.3 | 83.3 | 200.0 | 46534.3 |
| | STDER | 110.7 | 5.5 | 0.0 | 2300.9 |
| PBMC + LPS(5 ug/ml) + | 1st | 2824.0 | 110.0 | 200.0 | 52927.0 |
| YDE011 0.5 nM | 2nd | 2793.0 | 107.0 | 200.0 | 52269.8 |
| | 3rd | 2767.5 | 88.0 | 200.0 | 51730.5 |
| | Mean | 2794.8 | 101.7 | 200.0 | 52309.1 |
| | STDER | 16.3 | 6.9 | 0.0 | 346.0 |
| PBMC + LPS(5 ug/ml) + | 1st | 3224.0 | 105.0 | 200.0 | 61568.6 |
| YDE011 0.05 nM | 2nd | 3187.0 | 75.0 | 200.0 | 60755.7 |
| | 3rd | 3203.0 | 82.0 | 200.0 | 61106.8 |
| | Mean | 3204.7 | 87.3 | 200.0 | 61143.7 |
| | STDER | 10.7 | 9.1 | 0.0 | 235.4 |
| PBMC + LPS(5 ug/ml) + | 1st | 2963.5 | 76.0 | 200.0 | 55905.6 |
| YDE011 0.005 nM | 2nd | 2780.0 | 84.0 | 200.0 | 51994.7 |
| | 3rd | 2980.0 | 103.0 | 200.0 | 56260.2 |
| | Mean | 2907.8 | 87.7 | 200.0 | 54720.2 |
| | STDER | 64.1 | 8.0 | 0.0 | 1366.6 |
| PBMC + LPS(5 ug/ml) + | 1st | 2752.5 | 58.0 | 200.0 | 51413.7 |
| YDE043 500 nM | 2nd | 2670.0 | 97.0 | 200.0 | 49678.2 |
| | 3rd | 2861.0 | 86.0 | 200.0 | 53713.6 |
| | Mean | 2761.2 | 80.3 | 200.0 | 51601.8 |
| | STDER | 55.3 | 11.6 | 0.0 | 1168.7 |
| PBMC + LPS(5 ug/ml) + | 1st | 3049.5 | 72.0 | 200.0 | 57759.9 |
| YDE043 50 nM | 2nd | 3090.0 | 89.0 | 200.0 | 58638.2 |
| | 3rd | 2701.0 | 90.0 | 200.0 | 50329.0 |
| | Mean | 2946.8 | 83.7 | 200.0 | 55575.7 |
| | STDER | 123.5 | 5.8 | 0.0 | 2635.6 |
| PBMC + LPS(5 ug/ml) + | 1st | 2993.0 | 69.0 | 200.0 | 56540.0 |
| YDE043 5 nM | 2nd | 3073.0 | 69.0 | 200.0 | 58269.2 |
| | 3rd | 2846.0 | 105.0 | 200.0 | 53394.4 |
| | Mean | 2970.7 | 81.0 | 200.0 | 56067.9 |
| | STDER | 66.5 | 12.0 | 0.0 | 1426.9 |
| PBMC + LPS(5 ug/ml) + | 1st | 2928.0 | 66.0 | 200.0 | 55144.2 |
| YDE043 0.5 nM | 2nd | 2761.0 | 89.0 | 200.0 | 51593.2 |
| | 3rd | 2697.0 | 81.0 | 200.0 | 50244.9 |
| | Mean | 2795.3 | 78.7 | 200.0 | 52327.4 |
| | STDER | 68.9 | 6.7 | 0.0 | 1461.2 |
| PBMC + LPS(5 ug/ml) + | 1st | 2613.0 | 77.0 | 200.0 | 48485.5 |
| YDE043 0.05 nM | 2nd | 2832.5 | 60.0 | 200.0 | 53107.5 |
| | 3rd | 2773.0 | 89.0 | 200.0 | 51846.7 |
| | Mean | 2739.5 | 75.3 | 200.0 | 51146.6 |
| | STDER | 65.5 | 8.4 | 0.0 | 1379.4 |
| PBMC + LPS(5 ug/ml) + | 1st | 2977.5 | 86.0 | 200.0 | 56206.5 |
| YDE043 0.005 nM | 2nd | 2856.0 | 81.0 | 200.0 | 53607.2 |
| | 3rd | 3484.0 | 72.0 | 200.0 | 67372.3 |
| | Mean | 3105.8 | 79.7 | 200.0 | 59062.0 |
| | STDER | 192.3 | 4.1 | 0.0 | 4222.4 |

TABLE 12
Bead name: IL-10

| | | | | | Concen- |
|---|---|---|---|---|---|
| | | | | Dilu- | tration |
| Sample | | MFI | Count | tion | (pg/ml) |
| Blank | 1st | 63.5 | 64.0 | 1.0 | <2.59 |
| | 2nd | 61.0 | 61.0 | 1.0 | <2.59 |
| | Mean | 62.3 | 62.5 | 1.0 | #DIV/0! |
| | STDER | 1.3 | 1.5 | 0.0 | #DIV/0! |
| PBMC + 0.5% DMSO | 1st | 57.0 | 61.0 | 200.0 | <2.59 |
| | 2nd | 55.0 | 63.0 | 200.0 | <2.59 |
| | 3rd | 61.0 | 70.0 | 200.0 | <2.59 |
| | Mean | 57.7 | 64.7 | 200.0 | #DIV/0! |
| | STDER | 1.8 | 2.7 | 0.0 | #DIV/0! |
| PBMC + LPS(5 ug/ml) | 1st | 134.5 | 54.0 | 200.0 | 1536.4 |
| | 2nd | 127.5 | 50.0 | 200.0 | 1367.3 |
| | 3rd | 123.5 | 60.0 | 200.0 | 1269.7 |
| | Mean | 128.5 | 54.7 | 200.0 | 1391.1 |
| | STDER | 3.2 | 2.9 | 0.0 | 77.9 |
| PBMC + YY101 500 nM | 1st | 59.0 | 50.0 | 200.0 | <2.59 |
| | 2nd | 67.5 | 50.0 | 200.0 | <2.59 |
| | Mean | 63.3 | 50.0 | 200.0 | #DIV/0! |
| | STDER | 4.3 | 0.0 | 0.0 | #DIV/0! |
| PBMC + YY101 50 nM | 1st | 57.5 | 70.0 | 200.0 | <2.59 |
| | 2nd | 59.0 | 57.0 | 200.0 | <2.59 |
| | Mean | 58.3 | 63.5 | 200.0 | #DIV/0! |
| | STDER | 0.8 | 6.5 | 0.0 | #DIV/0! |
| PBMC + YY101 5 nM | 1st | 61.0 | 72.0 | 200.0 | <2.59 |
| | 2nd | 66.0 | 70.0 | 200.0 | <2.59 |
| | Mean | 63.5 | 71.0 | 200.0 | #DIV/0! |
| | STDER | 2.5 | 1.0 | 0.0 | #DIV/0! |
| PBMC + YY101 0.5 nM | 1st | 61.0 | 55.0 | 200.0 | <2.59 |
| | 2nd | 61.0 | 55.0 | 200.0 | <2.59 |
| | Mean | 61.0 | 55.0 | 200.0 | #DIV/0! |
| | STDER | 0.0 | 0.0 | 0.0 | #DIV/0! |
| PBMC + YY101 0.05 | 1st | 59.5 | 50.0 | 200.0 | <2.59 |
| uM | 2nd | 62.5 | 64.0 | 200.0 | <2.59 |
| | Mean | 61.0 | 57.0 | 200.0 | #DIV/0! |
| | STDER | 1.5 | 7.0 | 0.0 | #DIV/0! |
| PBMC + LPS(5 ug/ml) + | 1st | 114.0 | 67.0 | 200.0 | 1034.6 |
| Xiidra 500 nM | 2nd | 111.0 | 62.0 | 200.0 | 959.2 |
| | Mean | 112.5 | 64.5 | 200.0 | 996.9 |
| | STDER | 1.5 | 2.5 | 0.0 | 37.7 |
| PBMC + LPS(5 ug/ml) + | 1st | 126.0 | 51.0 | 200.0 | 1330.8 |
| Xiidra 50 nM | 2nd | 147.0 | 69.0 | 200.0 | 1833.9 |
| | Mean | 136.5 | 60.0 | 200.0 | 1582.3 |
| | STDER | 10.5 | 9.0 | 0.0 | 251.5 |
| PBMC + LPS(5 ug/ml) + | 1st | 135.0 | 70.0 | 200.0 | 1548.4 |
| Xiidra 5 nM | 2nd | 139.0 | 63.0 | 200.0 | 1644.1 |
| | Mean | 137.0 | 66.5 | 200.0 | 1596.3 |
| | STDER | 2.0 | 3.5 | 0.0 | 47.9 |
| PBMC + LPS(5 ug/ml) + | 1st | 114.5 | 66.0 | 200.0 | 1047.1 |
| Xiidra 0.5 nM | 2nd | 129.5 | 72.0 | 200.0 | 1415.8 |
| | Mean | 122.0 | 69.0 | 200.0 | 1231.5 |
| | STDER | 7.5 | 3.0 | 0.0 | 184.4 |
| PBMC + LPS(5 ug/ml) + | 1st | 119.0 | 78.0 | 200.0 | 1158.9 |
| Xiidra 0.05 nM | 2nd | 130.0 | 50.0 | 200.0 | 1427.9 |
| | Mean | 124.5 | 64.0 | 200.0 | 1293.4 |
| | STDER | 5.5 | 14.0 | 0.0 | 134.5 |
| PBMC + LPS(5 ug/ml) + | 1st | 117.5 | 50.0 | 200.0 | 1121.8 |
| Xiidra 0.005 nM | 2nd | 128.0 | 51.0 | 200.0 | 1379.4 |
| | Mean | 122.8 | 50.5 | 200.0 | 1250.6 |
| | STDER | 5.3 | 0.5 | 0.0 | 128.8 |
| PBMC + LPS(5 ug/ml) + | 1st | 106.0 | 71.0 | 200.0 | 832.3 |
| YY101 500 nM | 2nd | 108.0 | 69.0 | 200.0 | 883.3 |
| | 3rd | 103.0 | 50.0 | 200.0 | 755.1 |
| | Mean | 105.7 | 63.3 | 200.0 | 823.5 |
| | STDER | 1.5 | 6.7 | 0.0 | 37.3 |
| PBMC + LPS(5 ug/ml) + | 1st | 99.0 | 50.0 | 200.0 | 651.0 |
| YY101 50 nM | 2nd | 106.5 | 50.0 | 200.0 | 845.0 |
| | 3rd | 104.5 | 50.0 | 200.0 | 793.8 |
| | Mean | 103.3 | 50.0 | 200.0 | 763.3 |
| | STDER | 2.2 | 0.0 | 0.0 | 58.1 |
| PBMC + LPS(5 ug/ml) + | 1st | 107.0 | 65.0 | 200.0 | 857.8 |
| YY101 5 nM | 2nd | 110.0 | 52.0 | 200.0 | 934.0 |
| | 3rd | 113.0 | 50.0 | 200.0 | 1009.5 |
| | Mean | 110.0 | 55.7 | 200.0 | 933.8 |
| | STDER | 1.7 | 4.7 | 0.0 | 43.8 |
| PBMC + LPS(5 ug/ml) + | 1st | 107.5 | 62.0 | 200.0 | 870.5 |
| YY101 0.5 nM | 2nd | 101.0 | 54.0 | 200.0 | 703.2 |
| | 3rd | 101.5 | 58.0 | 200.0 | 716.2 |
| | Mean | 103.3 | 58.0 | 200.0 | 763.3 |
| | STDER | 2.1 | 2.3 | 0.0 | 53.7 |
| PBMC + LPS(5 ug/ml) + | 1st | 105.0 | 61.0 | 200.0 | 806.6 |
| YY101 0.05 nM | 2nd | 117.5 | 52.0 | 200.0 | 1121.8 |
| | 3rd | 108.0 | 54.0 | 200.0 | 883.3 |
| | Mean | 110.2 | 55.7 | 200.0 | 937.2 |
| | STDER | 3.8 | 2.7 | 0.0 | 94.9 |
| PBMC + LPS(5 ug/ml) + | 1st | 106.0 | 52.0 | 200.0 | 832.3 |
| YY101 0.005 nM | 2nd | 100.0 | 53.0 | 200.0 | 677.1 |
| | 3rd | 104.0 | 57.0 | 200.0 | 780.9 |
| | Mean | 103.3 | 54.0 | 200.0 | 763.4 |
| | STDER | 1.8 | 1.5 | 0.0 | 45.6 |
| PBMC + LPS(5 ug/ml) + | 1st | 108.0 | 70.0 | 200.0 | 883.3 |
| YDE011 500 nM | 2nd | 60.0 | 83.0 | 200.0 | <2.59 |
| | 3rd | 83.5 | 50.0 | 200.0 | 225.5 |
| | Mean | 83.8 | 67.7 | 200.0 | 554.4 |
| | STDER | 13.9 | 9.6 | 0.0 | 328.9 |
| PBMC + LPS(5 ug/ml) + | 1st | 94.0 | 67.0 | 200.0 | 518.2 |
| YDE011 50 nM | 2nd | 106.5 | 50.0 | 200.0 | 845.0 |
| | 3rd | 58.0 | 53.0 | 200.0 | <2.59 |
| | Mean | 86.2 | 56.7 | 200.0 | 681.6 |
| | STDER | 14.5 | 5.2 | 0.0 | 163.4 |
| PBMC + LPS(5 ug/ml) + | 1st | 82.0 | 73.0 | 200.0 | 181.1 |
| YDE011 5 nM | 2nd | 83.0 | 56.0 | 200.0 | 210.8 |
| | 3rd | 89.0 | 61.0 | 200.0 | 381.9 |
| | Mean | 84.7 | 63.3 | 200.0 | 257.9 |
| | STDER | 2.2 | 5.0 | 0.0 | 62.6 |
| PBMC + LPS(5 ug/ml) + | 1st | 90.0 | 51.0 | 200.0 | 409.5 |
| YDE011 0.5 nM | 2nd | 86.0 | 67.0 | 200.0 | 297.6 |
| | 3rd | 90.0 | 63.0 | 200.0 | 409.5 |
| | Mean | 88.7 | 60.3 | 200.0 | 372.2 |
| | STDER | 1.3 | 4.8 | 0.0 | 37.3 |
| PBMC + LPS(5 ug/ml) + | 1st | 102.0 | 71.0 | 200.0 | 729.2 |
| YDE011 0.05 nM | 2nd | 97.5 | 56.0 | 200.0 | 611.5 |
| | 3rd | 99.0 | 64.0 | 200.0 | 651.0 |
| | Mean | 99.5 | 63.7 | 200.0 | 663.9 |
| | STDER | 1.3 | 4.3 | 0.0 | 34.6 |
| PBMC + LPS(5 ug/ml) + | 1st | 98.0 | 60.0 | 200.0 | 624.7 |
| YDE011 0.005 nM | 2nd | 90.0 | 57.0 | 200.0 | 409.5 |
| | 3rd | 94.5 | 50.0 | 200.0 | 531.7 |
| | Mean | 94.2 | 55.7 | 200.0 | 521.9 |
| | STDER | 2.3 | 3.0 | 0.0 | 62.3 |
| PBMC + LPS(5 ug/ml) + | 1st | 92.0 | 54.0 | 200.0 | 464.2 |
| YDE043 500 nM | 2nd | 95.0 | 71.0 | 200.0 | 545.0 |
| | 3rd | 91.0 | 53.0 | 200.0 | 436.9 |
| | Mean | 92.7 | 59.3 | 200.0 | 482.0 |
| | STDER | 1.2 | 5.8 | 0.0 | 32.5 |
| PBMC + LPS(5 ug/ml) + | 1st | 96.0 | 64.0 | 200.0 | 571.7 |
| YDE043 50 nM | 2nd | 97.0 | 56.0 | 200.0 | 598.2 |
| | 3rd | 94.0 | 72.0 | 200.0 | 518.2 |
| | Mean | 95.7 | 64.0 | 200.0 | 562.7 |
| | STDER | 0.9 | 4.6 | 0.0 | 23.5 |
| PBMC + LPS(5 ug/ml) + | 1st | 95.0 | 54.0 | 200.0 | 545.0 |
| YDE043 5 nM | 2nd | 98.5 | 50.0 | 200.0 | 637.8 |
| | 3rd | 95.5 | 80.0 | 200.0 | 558.4 |
| | Mean | 96.3 | 61.3 | 200.0 | 580.4 |
| | STDER | 1.1 | 9.4 | 0.0 | 29.0 |
| PBMC + LPS(5 ug/ml) + | 1st | 91.0 | 55.0 | 200.0 | 436.9 |
| YDE043 0.5 nM | 2nd | 93.0 | 61.0 | 200.0 | 491.3 |
| | 3rd | 98.0 | 65.0 | 200.0 | 624.7 |
| | Mean | 94.0 | 60.3 | 200.0 | 517.6 |
| | STDER | 2.1 | 2.9 | 0.0 | 55.8 |
| PBMC + LPS(5 ug/ml) + | 1st | 91.0 | 57.0 | 200.0 | 436.9 |
| YDE043 0.05 nM | 2nd | 94.0 | 60.0 | 200.0 | 518.2 |
| | 3rd | 89.0 | 51.0 | 200.0 | 381.9 |
| | Mean | 91.3 | 56.0 | 200.0 | 445.7 |
| | STDER | 1.5 | 2.6 | 0.0 | 39.6 |
| PBMC + LPS(5 ug/ml) + | 1st | 99.0 | 62.0 | 200.0 | 651.0 |
| YDE043 0.005 nM | 2nd | 105.0 | 57.0 | 200.0 | 806.6 |
| | 3rd | 118.0 | 68.0 | 200.0 | 1134.2 |
| | Mean | 107.3 | 62.3 | 200.0 | 863.9 |
| | STDER | 5.6 | 3.2 | 0.0 | 142.4 |

TABLE 13
Bead name: TNF-alpha

| | | | | | Concen- |
|---|---|---|---|---|---|
| | | | | Dilu- | tration |
| Sample | | MFI | Count | tion | (pg/ml) |
| Blank | 1st | 19.0 | 79.0 | 1.0 | <7.70 |
| | 2nd | 21.0 | 89.0 | 1.0 | <7.70 |
| | Mean | 20.0 | 84.0 | 1.0 | #DIV/0! |
| | STDER | 1.0 | 5.0 | 0.0 | #DIV/0! |
| PBMC + 0.5% DMSO | 1st | 21.0 | 76.0 | 200.0 | <7.70 |
| | 2nd | 19.0 | 77.0 | 200.0 | <7.70 |
| | 3rd | 19.5 | 100.0 | 200.0 | <7.70 |
| | Mean | 19.8 | 84.3 | 200.0 | #DIV/0! |
| | STDER | 0.6 | 7.8 | 0.0 | #DIV/0! |
| PBMC + LPS(5 ug/ml) | 1st | 78.0 | 72.0 | 200.0 | 3554.3 |
| | 2nd | 66.0 | 87.0 | 200.0 | 2809.1 |
| | 3rd | 61.0 | 104.0 | 200.0 | 2495.5 |
| | Mean | 68.3 | 87.7 | 200.0 | 2953.0 |
| | STDER | 5.0 | 9.2 | 0.0 | 314.0 |
| PBMC + YY101 500 nM | 1st | 19.5 | 82.0 | 200.0 | <7.70 |
| | 2nd | 21.0 | 112.0 | 200.0 | <7.70 |
| | Mean | 20.3 | 97.0 | 200.0 | #DIV/0! |
| | STDER | 0.8 | 15.0 | 0.0 | #DIV/0! |
| PBMC + YY101 50 nM | 1st | 19.0 | 63.0 | 200.0 | <7.70 |
| | 2nd | 22.0 | 70.0 | 200.0 | <7.70 |
| | Mean | 20.5 | 66.5 | 200.0 | #DIV/0! |
| | STDER | 1.5 | 3.5 | 0.0 | #DIV/0! |
| PBMC + YY101 5 nM | 1st | 20.0 | 84.0 | 200.0 | <7.70 |
| | 2nd | 21.0 | 76.0 | 200.0 | <7.70 |
| | Mean | 20.5 | 80.0 | 200.0 | #DIV/0! |
| | STDER | 0.5 | 4.0 | 0.0 | #DIV/0! |
| PBMC + YY101 0.5 nM | 1st | 20.0 | 85.0 | 200.0 | <7.70 |
| | 2nd | 19.0 | 77.0 | 200.0 | <7.70 |
| | Mean | 19.5 | 81.0 | 200.0 | #DIV/0! |
| | STDER | 0.5 | 4.0 | 0.0 | #DIV/0! |
| PBMC + YY101 0.05 | 1st | 21.0 | 83.0 | 200.0 | <7.70 |
| uM | 2nd | 22.0 | 95.0 | 200.0 | <7.70 |
| | Mean | 21.5 | 89.0 | 200.0 | #DIV/0! |
| | STDER | 0.5 | 6.0 | 0.0 | #DIV/0! |
| PBMC + LPS(5 ug/ml) + | 1st | 72.0 | 71.0 | 200.0 | 3183.0 |
| Xiidra 500 nM | 2nd | 70.5 | 74.0 | 200.0 | 3089.8 |
| | Mean | 71.3 | 72.5 | 200.0 | 3136.4 |
| | STDER | 0.8 | 1.5 | 0.0 | 46.6 |
| PBMC + LPS(5 ug/ml) + | 1st | 71.0 | 85.0 | 200.0 | 3120.8 |
| Xiidra 50 nM | 2nd | 73.0 | 75.0 | 200.0 | 3245.0 |
| | Mean | 72.0 | 80.0 | 200.0 | 3182.9 |
| | STDER | 1.0 | 5.0 | 0.0 | 62.1 |
| PBMC + LPS(5 ug/ml) + | 1st | 63.0 | 80.0 | 200.0 | 2621.2 |
| Xiidra 5 nM | 2nd | 66.0 | 77.0 | 200.0 | 2809.1 |
| | Mean | 64.5 | 78.5 | 200.0 | 2715.2 |
| | STDER | 1.5 | 1.5 | 0.0 | 94.0 |
| PBMC + LPS(5 ug/ml) + | 1st | 59.0 | 86.0 | 200.0 | 2369.4 |
| Xiidra 0.5 nM | 2nd | 66.5 | 100.0 | 200.0 | 2840.4 |
| | Mean | 62.8 | 93.0 | 200.0 | 2604.9 |
| | STDER | 3.8 | 7.0 | 0.0 | 235.5 |
| PBMC + LPS(5 ug/ml) + | 1st | 56.0 | 79.0 | 200.0 | 2179.5 |
| Xiidra 0.05 nM | 2nd | 64.0 | 84.0 | 200.0 | 2683.9 |
| | Mean | 60.0 | 81.5 | 200.0 | 2431.7 |
| | STDER | 4.0 | 2.5 | 0.0 | 252.2 |
| PBMC + LPS(5 ug/ml) + | 1st | 61.0 | 82.0 | 200.0 | 2495.5 |
| Xiidra 0.005 nM | 2nd | 57.0 | 66.0 | 200.0 | 2242.9 |
| | Mean | 59.0 | 74.0 | 200.0 | 2369.2 |
| | STDER | 2.0 | 8.0 | 0.0 | 126.3 |
| PBMC + LPS(5 ug/ml) + | 1st | 56.0 | 77.0 | 200.0 | 2179.5 |
| YY101 500 nM | 2nd | 53.0 | 89.0 | 200.0 | 1988.8 |
| | 3rd | 50.0 | 88.0 | 200.0 | 1797.0 |
| | Mean | 53.0 | 84.7 | 200.0 | 1988.4 |
| | STDER | 1.7 | 3.8 | 0.0 | 110.4 |
| PBMC + LPS(5 ug/ml) + | 1st | 49.0 | 85.0 | 200.0 | 1732.8 |
| YY101 50 nM | 2nd | 48.0 | 83.0 | 200.0 | 1668.5 |
| | 3rd | 53.0 | 83.0 | 200.0 | 1988.8 |
| | Mean | 50.0 | 83.7 | 200.0 | 1796.7 |
| | STDER | 1.5 | 0.7 | 0.0 | 97.8 |
| PBMC + LPS(5 ug/ml) + | 1st | 53.0 | 69.0 | 200.0 | 1988.8 |
| YY101 5 nM | 2nd | 52.0 | 83.0 | 200.0 | 1925.0 |
| | 3rd | 57.0 | 60.0 | 200.0 | 2242.9 |
| | Mean | 54.0 | 70.7 | 200.0 | 2052.2 |
| | STDER | 1.5 | 6.7 | 0.0 | 97.1 |
| PBMC + LPS(5 ug/ml) + | 1st | 60.0 | 72.0 | 200.0 | 2432.5 |
| YY101 0.5 nM | 2nd | 57.0 | 93.0 | 200.0 | 2242.9 |
| | 3rd | 55.0 | 73.0 | 200.0 | 2116.1 |
| | Mean | 57.3 | 79.3 | 200.0 | 2263.8 |
| | STDER | 1.5 | 6.8 | 0.0 | 91.9 |
| PBMC + LPS(5 ug/ml) + | 1st | 50.0 | 78.0 | 200.0 | 1797.0 |
| YY101 0.05 nM | 2nd | 63.0 | 87.0 | 200.0 | 2621.2 |
| | 3rd | 57.0 | 71.0 | 200.0 | 2242.9 |
| | Mean | 56.7 | 78.7 | 200.0 | 2220.4 |
| | STDER | 3.8 | 4.6 | 0.0 | 238.2 |
| PBMC + LPS(5 ug/ml) + | 1st | 67.0 | 81.0 | 200.0 | 2871.6 |
| YY101 0.005 nM | 2nd | 59.0 | 79.0 | 200.0 | 2369.4 |
| | 3rd | 56.0 | 67.0 | 200.0 | 2179.5 |
| | Mean | 60.7 | 75.7 | 200.0 | 2473.5 |
| | STDER | 3.3 | 4.4 | 0.0 | 206.5 |
| PBMC + LPS(5 ug/ml) + | 1st | 55.0 | 85.0 | 200.0 | 2116.1 |
| YDE011 500 nM | 2nd | 32.5 | 84.0 | 200.0 | 647.8 |
| | 3rd | 41.5 | 80.0 | 200.0 | 1246.7 |
| | Mean | 43.0 | 83.0 | 200.0 | 1336.9 |
| | STDER | 6.5 | 1.5 | 0.0 | 426.2 |
| PBMC + LPS(5 ug/ml) + | 1st | 49.0 | 91.0 | 200.0 | 1732.8 |
| YDE011 50 nM | 2nd | 56.0 | 77.0 | 200.0 | 2179.5 |
| | 3rd | 29.5 | 82.0 | 200.0 | 442.1 |
| | Mean | 44.8 | 83.3 | 200.0 | 1451.5 |
| | STDER | 7.9 | 4.1 | 0.0 | 520.9 |
| PBMC + LPS(5 ug/ml) + | 1st | 34.0 | 95.0 | 200.0 | 749.3 |
| YDE011 5 nM | 2nd | 44.0 | 87.0 | 200.0 | 1409.7 |
| | 3rd | 48.0 | 82.0 | 200.0 | 1668.5 |
| | Mean | 42.0 | 88.0 | 200.0 | 1275.8 |
| | STDER | 4.2 | 3.8 | 0.0 | 273.7 |
| PBMC + LPS(5 ug/ml) + | 1st | 43.0 | 90.0 | 200.0 | 1344.6 |
| YDE011 0.5 nM | 2nd | 53.0 | 83.0 | 200.0 | 1988.8 |
| | 3rd | 50.0 | 81.0 | 200.0 | 1797.0 |
| | Mean | 48.7 | 84.7 | 200.0 | 1710.1 |
| | STDER | 3.0 | 2.7 | 0.0 | 191.0 |
| PBMC + LPS(5 ug/ml) + | 1st | 51.0 | 83.0 | 200.0 | 1861.0 |
| YDE011 0.05 nM | 2nd | 54.0 | 85.0 | 200.0 | 2052.5 |
| | 3rd | 54.0 | 87.0 | 200.0 | 2052.5 |
| | Mean | 53.0 | 85.0 | 200.0 | 1988.7 |
| | STDER | 1.0 | 1.2 | 0.0 | 63.8 |
| PBMC + LPS(5 ug/ml) + | 1st | 55.0 | 74.0 | 200.0 | 2116.1 |
| YDE011 0.005 nM | 2nd | 53.0 | 71.0 | 200.0 | 1988.8 |
| | 3rd | 51.0 | 98.0 | 200.0 | 1861.0 |
| | Mean | 53.0 | 81.0 | 200.0 | 1988.6 |
| | STDER | 1.2 | 8.5 | 0.0 | 73.6 |
| PBMC + LPS(5 ug/ml) + | 1st | 47.0 | 77.0 | 200.0 | 1604.0 |
| YDE043 500 nM | 2nd | 39.5 | 82.0 | 200.0 | 1115.3 |
| | 3rd | 43.0 | 80.0 | 200.0 | 1344.6 |
| | Mean | 43.2 | 79.7 | 200.0 | 1354.6 |
| | STDER | 2.2 | 1.5 | 0.0 | 141.2 |
| PBMC + LPS(5 ug/ml) + | 1st | 53.5 | 76.0 | 200.0 | 2020.6 |
| YDE043 50 nM | 2nd | 50.0 | 93.0 | 200.0 | 1797.0 |
| | 3rd | 45.0 | 88.0 | 200.0 | 1474.6 |
| | Mean | 49.5 | 85.7 | 200.0 | 1764.1 |
| | STDER | 2.5 | 5.0 | 0.0 | 158.5 |
| PBMC + LPS(5 ug/ml) + | 1st | 44.5 | 92.0 | 200.0 | 1442.2 |
| YDE043 5 nM | 2nd | 54.0 | 74.0 | 200.0 | 2052.5 |
| | 3rd | 45.0 | 120.0 | 200.0 | 1474.6 |
| | Mean | 47.8 | 95.3 | 200.0 | 1656.4 |
| | STDER | 3.1 | 13.4 | 0.0 | 198.2 |
| PBMC + LPS(5 ug/ml) + | 1st | 49.5 | 54.0 | 200.0 | 1764.9 |
| YDE043 0.5 nM | 2nd | 44.0 | 85.0 | 200.0 | 1409.7 |
| | 3rd | 44.0 | 73.0 | 200.0 | 1409.7 |
| | Mean | 45.8 | 70.7 | 200.0 | 1528.1 |
| | STDER | 1.8 | 9.0 | 0.0 | 118.4 |
| PBMC + LPS(5 ug/ml) + | 1st | 40.5 | 82.0 | 200.0 | 1181.1 |
| YDE043 0.05 nM | 2nd | 46.0 | 79.0 | 200.0 | 1539.4 |
| | 3rd | 45.0 | 80.0 | 200.0 | 1474.6 |
| | Mean | 43.8 | 80.3 | 200.0 | 1398.4 |
| | STDER | 1.7 | 0.9 | 0.0 | 110.2 |
| PBMC + LPS(5 ug/ml) + | 1st | 54.0 | 80.0 | 200.0 | 2052.5 |
| YDE043 0.005 nM | 2nd | 50.0 | 81.0 | 200.0 | 1797.0 |
| | 3rd | 58.0 | 95.0 | 200.0 | 2306.2 |
| | Mean | 54.0 | 85.3 | 200.0 | 2051.9 |
| | STDER | 2.3 | 4.8 | 0.0 | 147.0 |

TABLE 14
Bead name: MMP-3

| | | | | | Concen- |
|---|---|---|---|---|---|
| | | | | Dilu- | tration |
| Sample | | MFI | Count | tion | (pg/ml) |
| Blank | 1st | 48.0 | 55.0 | 1.0 | 29.2 |
| | 2nd | 48.0 | 65.0 | 1.0 | 29.2 |
| | Mean | 48.0 | 60.0 | 1.0 | 29.2 |
| | STDER | 0.0 | 5.0 | 0.0 | 0.0 |
| PBMC + 0.5% DMSO | 1st | 51.0 | 64.0 | 200.0 | 6062.8 |
| | 2nd | 47.0 | 67.0 | 200.0 | 5771.5 |
| | 3rd | 49.5 | 58.0 | 200.0 | 5953.3 |
| | Mean | 49.2 | 63.0 | 200.0 | 5929.2 |
| | STDER | 1.2 | 2.6 | 0.0 | 84.9 |
| PBMC + LPS(5 ug/ml) | 1st | 96.5 | 50.0 | 200.0 | 9511.1 |
| | 2nd | 87.0 | 61.0 | 200.0 | 8772.5 |
| | 3rd | 84.0 | 53.0 | 200.0 | 8541.1 |
| | Mean | 89.2 | 54.7 | 200.0 | 8941.6 |
| | STDER | 3.8 | 3.3 | 0.0 | 292.5 |
| PBMC + YY101 500 nM | 1st | 54.0 | 82.0 | 200.0 | 6282.7 |
| | 2nd | 56.0 | 81.0 | 200.0 | 6429.9 |
| | Mean | 55.0 | 81.5 | 200.0 | 6356.3 |
| | STDER | 1.0 | 0.5 | 0.0 | 73.6 |
| PBMC + YY101 50 nM | 1st | 54.0 | 66.0 | 200.0 | 6282.7 |
| | 2nd | 49.5 | 50.0 | 200.0 | 5953.3 |
| | Mean | 51.8 | 58.0 | 200.0 | 6118.0 |
| | STDER | 2.3 | 8.0 | 0.0 | 164.7 |
| PBMC + YY101 5 nM | 1st | 52.5 | 64.0 | 200.0 | 6172.6 |
| | 2nd | 54.5 | 50.0 | 200.0 | 6319.4 |
| | Mean | 53.5 | 57.0 | 200.0 | 6246.0 |
| | STDER | 1.0 | 7.0 | 0.0 | 73.4 |
| PBMC + YY101 0.5 nM | 1st | 51.0 | 68.0 | 200.0 | 6062.8 |
| | 2nd | 56.0 | 59.0 | 200.0 | 6429.9 |
| | Mean | 53.5 | 63.5 | 200.0 | 6246.3 |
| | STDER | 2.5 | 4.5 | 0.0 | 183.5 |
| PBMC + YY101 0.05 | 1st | 57.0 | 53.0 | 200.0 | 6503.7 |
| uM | 2nd | 53.5 | 64.0 | 200.0 | 6245.9 |
| | Mean | 55.3 | 58.5 | 200.0 | 6374.8 |
| | STDER | 1.8 | 5.5 | 0.0 | 128.9 |
| PBMC + LPS(5 ug/ml) + | 1st | 93.0 | 55.0 | 200.0 | 9237.9 |
| Xiidra 500 nM | 2nd | 87.5 | 56.0 | 200.0 | 8811.1 |
| | Mean | 90.3 | 55.5 | 200.0 | 9024.5 |
| | STDER | 2.8 | 0.5 | 0.0 | 213.4 |
| PBMC + LPS(5 ug/ml) + | 1st | 93.0 | 54.0 | 200.0 | 9237.9 |
| Xiidra 50 nM | 2nd | 99.0 | 61.0 | 200.0 | 9707.0 |
| | Mean | 96.0 | 57.5 | 200.0 | 9472.4 |
| | STDER | 3.0 | 3.5 | 0.0 | 234.5 |
| PBMC + LPS(5 ug/ml) + | 1st | 90.0 | 73.0 | 200.0 | 9004.8 |
| Xiidra 5 nM | 2nd | 97.0 | 67.0 | 200.0 | 9550.2 |
| | Mean | 93.5 | 70.0 | 200.0 | 9277.5 |
| | STDER | 3.5 | 3.0 | 0.0 | 272.7 |
| PBMC + LPS(5 ug/ml) + | 1st | 79.0 | 84.0 | 200.0 | 8157.7 |
| Xiidra 0.5 nM | 2nd | 87.0 | 63.0 | 200.0 | 8772.5 |
| | Mean | 83.0 | 73.5 | 200.0 | 8465.1 |
| | STDER | 4.0 | 10.5 | 0.0 | 307.4 |
| PBMC + LPS(5 ug/ml) + | 1st | 81.0 | 70.0 | 200.0 | 8310.7 |
| Xiidra 0.05 nM | 2nd | 85.0 | 73.0 | 200.0 | 8618.2 |
| | Mean | 83.0 | 71.5 | 200.0 | 8464.4 |
| | STDER | 2.0 | 1.5 | 0.0 | 153.7 |
| PBMC + LPS(5 ug/ml) + | 1st | 81.0 | 53.0 | 200.0 | 8310.7 |
| Xiidra 0.005 nM | 2nd | 83.5 | 64.0 | 200.0 | 8502.7 |
| | Mean | 82.3 | 58.5 | 200.0 | 8406.7 |
| | STDER | 1.3 | 5.5 | 0.0 | 96.0 |
| PBMC + LPS(5 ug/ml) + | 1st | 77.5 | 60.0 | 200.0 | 8043.1 |
| YY101 500 nM | 2nd | 72.5 | 68.0 | 200.0 | 7663.2 |
| | 3rd | 71.0 | 62.0 | 200.0 | 7549.7 |
| | Mean | 73.7 | 63.3 | 200.0 | 7752.0 |
| | STDER | 2.0 | 2.4 | 0.0 | 149.2 |
| PBMC + LPS(5 ug/ml) + | 1st | 71.0 | 53.0 | 200.0 | 7549.7 |
| YY101 50 nM | 2nd | 73.0 | 61.0 | 200.0 | 7701.1 |
| | 3rd | 78.0 | 57.0 | 200.0 | 8081.3 |
| | Mean | 74.0 | 57.0 | 200.0 | 7777.4 |
| | STDER | 2.1 | 2.3 | 0.0 | 158.1 |
| PBMC + LPS(5 ug/ml) + | 1st | 71.0 | 78.0 | 200.0 | 7549.7 |
| YY101 5 nM | 2nd | 71.0 | 54.0 | 200.0 | 7549.7 |
| | 3rd | 85.0 | 52.0 | 200.0 | 8618.2 |
| | Mean | 75.7 | 61.3 | 200.0 | 7905.9 |
| | STDER | 4.7 | 8.4 | 0.0 | 356.1 |
| PBMC + LPS(5 ug/ml) + | 1st | 73.0 | 87.0 | 200.0 | 7701.1 |
| YY101 0.5 nM | 2nd | 74.0 | 60.0 | 200.0 | 7776.9 |
| | 3rd | 74.0 | 53.0 | 200.0 | 7776.9 |
| | Mean | 73.7 | 66.7 | 200.0 | 7751.6 |
| | STDER | 0.3 | 10.4 | 0.0 | 25.3 |
| PBMC + LPS(5 ug/ml) + | 1st | 74.0 | 53.0 | 200.0 | 7776.9 |
| YY101 0.05 nM | 2nd | 84.0 | 51.0 | 200.0 | 8541.1 |
| | 3rd | 76.0 | 67.0 | 200.0 | 7928.9 |
| | Mean | 78.0 | 57.0 | 200.0 | 8082.3 |
| | STDER | 3.1 | 5.0 | 0.0 | 233.6 |
| PBMC + LPS(5 ug/ml) + | 1st | 71.0 | 51.0 | 200.0 | 7549.7 |
| YY101 0.005 nM | 2nd | 70.0 | 60.0 | 200.0 | 7474.3 |
| | 3rd | 72.0 | 69.0 | 200.0 | 7625.3 |
| | Mean | 71.0 | 60.0 | 200.0 | 7549.8 |
| | STDER | 0.6 | 5.2 | 0.0 | 43.6 |
| PBMC + LPS(5 ug/ml) + | 1st | 74.0 | 50.0 | 200.0 | 7776.9 |
| YDE011 500 nM | 2nd | 41.5 | 50.0 | 200.0 | 5374.6 |
| | 3rd | 60.0 | 55.0 | 200.0 | 6725.8 |
| | Mean | 58.5 | 51.7 | 200.0 | 6625.8 |
| | STDER | 9.4 | 1.7 | 0.0 | 695.3 |
| PBMC + LPS(5 ug/ml) + | 1st | 67.0 | 63.0 | 200.0 | 7248.5 |
| YDE011 50 nM | 2nd | 70.0 | 53.0 | 200.0 | 7474.3 |
| | 3rd | 43.0 | 63.0 | 200.0 | 5482.5 |
| | Mean | 60.0 | 59.7 | 200.0 | 6735.1 |
| | STDER | 8.5 | 3.3 | 0.0 | 629.7 |
| PBMC + LPS(5 ug/ml) + | 1st | 54.0 | 50.0 | 200.0 | 6282.7 |
| YDE011 5 nM | 2nd | 61.0 | 58.0 | 200.0 | 6800.1 |
| | 3rd | 68.0 | 63.0 | 200.0 | 7323.6 |
| | Mean | 61.0 | 57.0 | 200.0 | 6802.1 |
| | STDER | 4.0 | 3.8 | 0.0 | 300.5 |
| PBMC + LPS(5 ug/ml) + | 1st | 67.0 | 87.0 | 200.0 | 7248.5 |
| YDE011 0.5 nM | 2nd | 65.0 | 50.0 | 200.0 | 7098.6 |
| | 3rd | 64.5 | 70.0 | 200.0 | 7061.2 |
| | Mean | 65.5 | 69.0 | 200.0 | 7136.1 |
| | STDER | 0.8 | 10.7 | 0.0 | 57.2 |
| PBMC + LPS(5 ug/ml) + | 1st | 70.0 | 67.0 | 200.0 | 7474.3 |
| YDE011 0.05 nM | 2nd | 76.0 | 57.0 | 200.0 | 7928.9 |
| | 3rd | 70.5 | 50.0 | 200.0 | 7512.0 |
| | Mean | 72.2 | 58.0 | 200.0 | 7638.4 |
| | STDER | 1.9 | 4.9 | 0.0 | 145.7 |
| PBMC + LPS(5 ug/ml) + | 1st | 70.5 | 66.0 | 200.0 | 7512.0 |
| YDE011 0.005 nM | 2nd | 68.0 | 67.0 | 200.0 | 7323.6 |
| | 3rd | 66.0 | 63.0 | 200.0 | 7173.5 |
| | Mean | 68.2 | 65.3 | 200.0 | 7336.4 |
| | STDER | 1.3 | 1.2 | 0.0 | 97.9 |
| PBMC + LPS(5 ug/ml) + | 1st | 65.0 | 57.0 | 200.0 | 7098.6 |
| YDE043 500 nM | 2nd | 72.5 | 56.0 | 200.0 | 7663.2 |
| | 3rd | 66.0 | 71.0 | 200.0 | 7173.5 |
| | Mean | 67.8 | 61.3 | 200.0 | 7311.7 |
| | STDER | 2.4 | 4.8 | 0.0 | 177.0 |
| PBMC + LPS(5 ug/ml) + | 1st | 73.0 | 53.0 | 200.0 | 7701.1 |
| YDE043 50 nM | 2nd | 69.0 | 51.0 | 200.0 | 7398.9 |
| | 3rd | 62.5 | 50.0 | 200.0 | 6911.8 |
| | Mean | 68.2 | 51.3 | 200.0 | 7337.3 |
| | STDER | 3.1 | 0.9 | 0.0 | 229.9 |
| PBMC + LPS(5 ug/ml) + | 1st | 70.0 | 55.0 | 200.0 | 7474.3 |
| YDE043 5 nM | 2nd | 70.0 | 61.0 | 200.0 | 7474.3 |
| | 3rd | 68.0 | 75.0 | 200.0 | 7323.6 |
| | Mean | 69.3 | 63.7 | 200.0 | 7424.0 |
| | STDER | 0.7 | 5.9 | 0.0 | 50.2 |
| PBMC + LPS(5 ug/ml) + | 1st | 65.0 | 58.0 | 200.0 | 7098.6 |
| YDE043 0.5 nM | 2nd | 67.0 | 69.0 | 200.0 | 7248.5 |
| | 3rd | 66.0 | 65.0 | 200.0 | 7173.5 |
| | Mean | 66.0 | 64.0 | 200.0 | 7173.5 |
| | STDER | 0.6 | 3.2 | 0.0 | 43.3 |
| PBMC + LPS(5 ug/ml) + | 1st | 64.5 | 60.0 | 200.0 | 7061.2 |
| YDE043 0.05 nM | 2nd | 67.0 | 50.0 | 200.0 | 7248.5 |
| | 3rd | 65.0 | 73.0 | 200.0 | 7098.6 |
| | Mean | 65.5 | 61.0 | 200.0 | 7136.1 |
| | STDER | 0.8 | 6.7 | 0.0 | 57.2 |
| PBMC + LPS(5 ug/ml) + | 1st | 66.0 | 55.0 | 200.0 | 7173.5 |
| YDE043 0.005 nM | 2nd | 74.0 | 50.0 | 200.0 | 7776.9 |
| | 3rd | 78.0 | 61.0 | 200.0 | 8081.3 |
| | Mean | 72.7 | 55.3 | 200.0 | 7677.2 |
| | STDER | 3.5 | 3.2 | 0.0 | 266.8 |

TABLE 15
Bead name: CCL2/MCP-1

| | | | | | Concentration |
|---|---|---|---|---|---|
| Sample | | MFI | Count | Dilution | (pg/ml) |
| Blank | 1st | 18.0 | 79.0 | 1.0 | 21.6 |
| | 2nd | 19.0 | 96.0 | 1.0 | 22.1 |
| | Mean | 18.5 | 87.5 | 1.0 | 21.9 |
| | STDER | 0.5 | 8.5 | 0.0 | 0.3 |
| PBMC + | 1st | 17.0 | 73.0 | 200.0 | 4216.3 |
| 0.5% DMSO | 2nd | 18.0 | 81.0 | 200.0 | 4321.5 |
| | 3rd | 18.0 | 107.0 | 200.0 | 4321.5 |
| | Mean | 17.7 | 87.0 | 200.0 | 4286.5 |
| | STDER | 0.3 | 10.3 | 0.0 | 35.1 |
| PBMC + | 1st | 36.0 | 78.0 | 200.0 | 6053.2 |
| LPS(5 ug/ml) | 2nd | 31.0 | 86.0 | 200.0 | 5597.9 |
| | 3rd | 28.0 | 93.0 | 200.0 | 5316.3 |
| | Mean | 31.7 | 85.7 | 200.0 | 5655.8 |
| | STDER | 2.3 | 4.3 | 0.0 | 214.7 |
| PBMC + | 1st | 18.0 | 83.0 | 200.0 | 4321.5 |
| YY101 500 nM | 2nd | 21.0 | 111.0 | 200.0 | 4630.2 |
| | Mean | 19.5 | 97.0 | 200.0 | 4475.9 |
| | STDER | 1.5 | 14.0 | 0.0 | 154.4 |
| PBMC + | 1st | 18.0 | 86.0 | 200.0 | 4321.5 |
| YY101 50 nM | 2nd | 19.0 | 87.0 | 200.0 | 4425.5 |
| | Mean | 18.5 | 86.5 | 200.0 | 4373.5 |
| | STDER | 0.5 | 0.5 | 0.0 | 52.0 |
| PBMC + | 1st | 19.0 | 106.0 | 200.0 | 4425.5 |
| YY101 5 nM | 2nd | 19.0 | 93.0 | 200.0 | 4425.5 |
| | Mean | 19.0 | 99.5 | 200.0 | 4425.5 |
| | STDER | 0.0 | 6.5 | 0.0 | 0.0 |
| PBMC + | 1st | 20.0 | 78.0 | 200.0 | 4528.4 |
| YY101 0.5 nM | 2nd | 23.0 | 90.0 | 200.0 | 4830.8 |
| | Mean | 21.5 | 84.0 | 200.0 | 4679.6 |
| | STDER | 1.5 | 6.0 | 0.0 | 151.2 |
| PBMC + | 1st | 33.0 | 69.0 | 200.0 | 5782.0 |
| YY101 0.05 uM | 2nd | 17.5 | 96.0 | 200.0 | 4269.1 |
| | Mean | 25.3 | 82.5 | 200.0 | 5025.5 |
| | STDER | 7.8 | 13.5 | 0.0 | 756.4 |
| PBMC + | 1st | 34.0 | 94.0 | 200.0 | 5873.0 |
| LPS(5 ug/ml) + | 2nd | 32.0 | 76.0 | 200.0 | 5690.3 |
| Xiidra 500 nM | Mean | 33.0 | 85.0 | 200.0 | 5781.6 |
| | STDER | 1.0 | 9.0 | 0.0 | 91.4 |
| PBMC + | 1st | 35.0 | 88.0 | 200.0 | 5963.4 |
| LPS(5 ug/ml) + | 2nd | 36.0 | 101.0 | 200.0 | 6053.2 |
| Xiidra 50 nM | Mean | 35.5 | 94.5 | 200.0 | 6008.3 |
| | STDER | 0.5 | 6.5 | 0.0 | 44.9 |
| PBMC + | 1st | 27.0 | 90.0 | 200.0 | 5220.9 |
| LPS(5 ug/ml) + | 2nd | 35.5 | 90.0 | 200.0 | 6008.4 |
| Xiidra 5 nM | Mean | 31.3 | 90.0 | 200.0 | 5614.6 |
| | STDER | 4.3 | 0.0 | 0.0 | 393.7 |
| PBMC + | 1st | 28.0 | 92.0 | 200.0 | 5316.3 |
| LPS(5 ug/ml) + | 2nd | 27.5 | 90.0 | 200.0 | 5268.7 |
| Xiidra 0.5 nM | Mean | 27.8 | 91.0 | 200.0 | 5292.5 |
| | STDER | 0.3 | 1.0 | 0.0 | 23.8 |
| PBMC + | 1st | 30.0 | 91.0 | 200.0 | 5504.7 |
| LPS(5 ug/ml) + | 2nd | 28.0 | 86.0 | 200.0 | 5316.3 |
| Xiidra 0.05 nM | Mean | 29.0 | 88.5 | 200.0 | 5410.5 |
| | STDER | 1.0 | 2.5 | 0.0 | 94.2 |
| PBMC + | 1st | 27.0 | 93.0 | 200.0 | 5220.9 |
| LPS(5 ug/ml) + | 2nd | 28.0 | 70.0 | 200.0 | 5316.3 |
| Xiidra 0.005 nM | Mean | 27.5 | 81.5 | 200.0 | 5268.6 |
| | STDER | 0.5 | 11.5 | 0.0 | 47.7 |
| PBMC + | 1st | 19.0 | 69.0 | 200.0 | 4425.5 |
| LPS(5 ug/ml) + | 2nd | 19.0 | 94.0 | 200.0 | 4425.5 |
| YY101 500 nM | 3rd | 20.0 | 107.0 | 200.0 | 4528.4 |
| | Mean | 19.3 | 90.0 | 200.0 | 4459.8 |
| | STDER | 0.3 | 11.2 | 0.0 | 34.3 |
| PBMC + | 1st | 20.5 | 82.0 | 200.0 | 4579.5 |
| LPS(5 ug/ml) + | 2nd | 21.0 | 81.0 | 200.0 | 4630.2 |
| YY101 50 nM | 3rd | 21.0 | 85.0 | 200.0 | 4630.2 |
| | Mean | 20.8 | 82.7 | 200.0 | 4613.3 |
| | STDER | 0.2 | 1.2 | 0.0 | 16.9 |
| PBMC + | 1st | 21.0 | 106.0 | 200.0 | 4630.2 |
| LPS(5 ug/ml) + | 2nd | 20.0 | 81.0 | 200.0 | 4528.4 |
| YY101 5 nM | 3rd | 22.0 | 71.0 | 200.0 | 4731.0 |
| | Mean | 21.0 | 86.0 | 200.0 | 4629.9 |
| | STDER | 0.6 | 10.4 | 0.0 | 58.5 |
| PBMC + | 1st | 21.0 | 98.0 | 200.0 | 4630.2 |
| LPS(5 ug/ml) + | 2nd | 20.0 | 82.0 | 200.0 | 4528.4 |
| YY101 0.5 nM | 3rd | 21.0 | 85.0 | 200.0 | 4630.2 |
| | Mean | 20.7 | 88.3 | 200.0 | 4596.3 |
| | STDER | 0.3 | 4.9 | 0.0 | 33.9 |
| PBMC + | 1st | 20.0 | 87.0 | 200.0 | 4528.4 |
| LPS(5 ug/ml) + | 2nd | 22.0 | 86.0 | 200.0 | 4731.0 |
| YY101 0.05 nM | 3rd | 20.0 | 94.0 | 200.0 | 4528.4 |
| | Mean | 20.7 | 89.0 | 200.0 | 4596.0 |
| | STDER | 0.7 | 2.5 | 0.0 | 67.5 |
| PBMC + | 1st | 20.0 | 89.0 | 200.0 | 4528.4 |
| LPS(5 ug/ml) + | 2nd | 21.0 | 88.0 | 200.0 | 4630.2 |
| YY101 0.005 nM | 3rd | 19.0 | 74.0 | 200.0 | 4425.5 |
| | Mean | 20.0 | 83.7 | 200.0 | 4528.1 |
| | STDER | 0.6 | 4.8 | 0.0 | 59.1 |
| PBMC + | 1st | 22.0 | 89.0 | 200.0 | 4731.0 |
| LPS(5 ug/ml) + | 2nd | 12.0 | 101.0 | 200.0 | 3670.6 |
| YDE011 500 nM | 3rd | 16.0 | 91.0 | 200.0 | 4109.9 |
| | Mean | 16.7 | 93.7 | 200.0 | 4170.5 |
| | STDER | 2.9 | 3.7 | 0.0 | 307.6 |
| PBMC + | 1st | 18.0 | 89.0 | 200.0 | 4321.5 |
| LPS(5 ug/ml) + | 2nd | 20.0 | 79.0 | 200.0 | 4528.4 |
| YDE011 50 nM | 3rd | 12.0 | 74.0 | 200.0 | 3670.6 |
| | Mean | 16.7 | 80.7 | 200.0 | 4173.5 |
| | STDER | 2.4 | 4.4 | 0.0 | 258.5 |
| PBMC + | 1st | 14.0 | 92.0 | 200.0 | 3893.1 |
| LPS(5 ug/ml) + | 2nd | 18.0 | 88.0 | 200.0 | 4321.5 |
| YDE011 5 nM | 3rd | 18.0 | 103.0 | 200.0 | 4321.5 |
| | Mean | 16.7 | 94.3 | 200.0 | 4178.7 |
| | STDER | 1.3 | 4.5 | 0.0 | 142.8 |
| PBMC + | 1st | 19.0 | 115.0 | 200.0 | 4425.5 |
| LPS(5 ug/ml) + | 2nd | 17.5 | 86.0 | 200.0 | 4269.1 |
| YDE011 0.5 nM | 3rd | 17.0 | 97.0 | 200.0 | 4216.3 |
| | Mean | 17.8 | 99.3 | 200.0 | 4303.7 |
| | STDER | 0.6 | 8.5 | 0.0 | 62.8 |
| PBMC + | 1st | 19.0 | 95.0 | 200.0 | 4425.5 |
| LPS(5 ug/ml) + | 2nd | 19.0 | 71.0 | 200.0 | 4425.5 |
| YDE011 0.05 nM | 3rd | 19.5 | 94.0 | 200.0 | 4477.1 |
| | Mean | 19.2 | 86.7 | 200.0 | 4442.7 |
| | STDER | 0.2 | 7.8 | 0.0 | 17.2 |
| PBMC + | 1st | 19.0 | 69.0 | 200.0 | 4425.5 |
| LPS(5 ug/ml) + | 2nd | 20.0 | 92.0 | 200.0 | 4528.4 |
| YDE011 0.005 nM | 3rd | 18.0 | 101.0 | 200.0 | 4321.5 |
| | Mean | 19.0 | 87.3 | 200.0 | 4425.2 |
| | STDER | 0.6 | 9.5 | 0.0 | 59.7 |
| PBMC + | 1st | 21.0 | 90.0 | 200.0 | 4630.2 |
| LPS(5 ug/ml) + | 2nd | 21.0 | 92.0 | 200.0 | 4630.2 |
| YDE043 500 nM | 3rd | 21.0 | 88.0 | 200.0 | 4630.2 |
| | Mean | 21.0 | 90.0 | 200.0 | 4630.2 |
| | STDER | 0.0 | 1.2 | 0.0 | 0.0 |
| PBMC + | 1st | 20.0 | 92.0 | 200.0 | 4528.4 |
| LPS(5 ug/ml) + | 2nd | 22.0 | 90.0 | 200.0 | 4731.0 |
| YDE043 50 nM | 3rd | 19.5 | 72.0 | 200.0 | 4477.1 |
| | Mean | 20.5 | 84.7 | 200.0 | 4578.9 |
| | STDER | 0.8 | 6.4 | 0.0 | 77.5 |
| PBMC + | 1st | 20.5 | 74.0 | 200.0 | 4579.5 |
| LPS(5 ug/ml) + | 2nd | 19.5 | 68.0 | 200.0 | 4477.1 |
| YDE043 5 nM | 3rd | 21.0 | 103.0 | 200.0 | 4630.2 |
| | Mean | 20.3 | 81.7 | 200.0 | 4562.3 |
| | STDER | 0.4 | 10.8 | 0.0 | 45.0 |
| PBMC + | 1st | 19.0 | 71.0 | 200.0 | 4425.5 |
| LPS(5 ug/ml) + | 2nd | 18.0 | 86.0 | 200.0 | 4321.5 |
| YDE043 0.5 nM | 3rd | 19.0 | 91.0 | 200.0 | 4425.5 |
| | Mean | 18.7 | 82.7 | 200.0 | 4390.9 |
| | STDER | 0.3 | 6.0 | 0.0 | 34.7 |
| PBMC + | 1st | 20.0 | 95.0 | 200.0 | 4528.4 |
| LPS(5 ug/ml) + | 2nd | 19.5 | 76.0 | 200.0 | 4477.1 |
| YDE043 0.05 nM | 3rd | 21.0 | 93.0 | 200.0 | 4630.2 |
| | Mean | 20.2 | 88.0 | 200.0 | 4545.3 |
| | STDER | 0.4 | 6.0 | 0.0 | 45.0 |
| PBMC + | 1st | 19.0 | 71.0 | 200.0 | 4425.5 |
| LPS(5 ug/ml) + | 2nd | 22.0 | 78.0 | 200.0 | 4731.0 |
| YDE043 0.005 nM | 3rd | 22.0 | 99.0 | 200.0 | 4731.0 |
| | Mean | 21.0 | 82.7 | 200.0 | 4629.2 |
| | STDER | 1.0 | 8.4 | 0.0 | 101.8 |

TABLE 16
Bead name: CCL3/MIP-1 alpha

| | | | | | Concentration |
|---|---|---|---|---|---|
| Sample | | MFI | Count | Dilution | (pg/ml) |
| Blank | 1st | 17.0 | 64.0 | 1.0 | 70.1 |
| | 2nd | 16.0 | 70.0 | 1.0 | 65.0 |
| | Mean | 16.5 | 67.0 | 1.0 | 67.5 |
| | STDER | 0.5 | 3.0 | 0.0 | 2.6 |
| PBMC + | 1st | 18.0 | 67.0 | 200.0 | 14981.7 |
| 0.5% DMSO | 2nd | 18.5 | 58.0 | 200.0 | 15447.9 |
| | 3rd | 19.5 | 62.0 | 200.0 | 16349.6 |
| | Mean | 18.7 | 62.3 | 200.0 | 15593.1 |
| | STDER | 0.4 | 2.6 | 0.0 | 401.5 |
| PBMC + | 1st | 96.0 | 55.0 | 200.0 | 54195.6 |
| LPS(5 ug/ml) | 2nd | 66.0 | 55.0 | 200.0 | 42643.2 |
| | 3rd | 67.0 | 59.0 | 200.0 | 43065.7 |
| | Mean | 76.3 | 56.3 | 200.0 | 46634.8 |
| | STDER | 9.8 | 1.3 | 0.0 | 3782.4 |
| PBMC + | 1st | 17.0 | 77.0 | 200.0 | 14014.0 |
| YY101 500 nM | 2nd | 17.0 | 87.0 | 200.0 | 14014.0 |
| | Mean | 17.0 | 82.0 | 200.0 | 14014.0 |
| | STDER | 0.0 | 5.0 | 0.0 | 0.0 |
| PBMC + | 1st | 16.5 | 50.0 | 200.0 | 13510.6 |
| YY101 50 nM | 2nd | 18.0 | 54.0 | 200.0 | 14981.7 |
| | Mean | 17.3 | 52.0 | 200.0 | 14246.1 |
| | STDER | 0.8 | 2.0 | 0.0 | 735.6 |
| PBMC + | 1st | 17.0 | 54.0 | 200.0 | 14014.0 |
| YY101 5 nM | 2nd | 17.5 | 62.0 | 200.0 | 14504.0 |
| | Mean | 17.3 | 58.0 | 200.0 | 14259.0 |
| | STDER | 0.3 | 4.0 | 0.0 | 245.0 |
| PBMC + | 1st | 19.0 | 50.0 | 200.0 | 15903.7 |
| YY101 0.5 nM | 2nd | 20.0 | 61.0 | 200.0 | 16786.4 |
| | Mean | 19.5 | 55.5 | 200.0 | 16345.0 |
| | STDER | 0.5 | 5.5 | 0.0 | 441.4 |
| PBMC + | 1st | 19.0 | 58.0 | 200.0 | 15903.7 |
| YY101 0.05 uM | 2nd | 15.0 | 50.0 | 200.0 | 11904.9 |
| | Mean | 17.0 | 54.0 | 200.0 | 13904.3 |
| | STDER | 2.0 | 4.0 | 0.0 | 1999.4 |
| PBMC + | 1st | 81.0 | 63.0 | 200.0 | 48681.5 |
| LPS(5 ug/ml) + | 2nd | 77.5 | 54.0 | 200.0 | 47325.4 |
| Xiidra 500 nM | Mean | 79.3 | 58.5 | 200.0 | 48003.4 |
| | STDER | 1.8 | 4.5 | 0.0 | 678.0 |
| PBMC + | 1st | 77.0 | 56.0 | 200.0 | 47129.3 |
| LPS(5 ug/ml) + | 2nd | 79.5 | 50.0 | 200.0 | 48103.9 |
| Xiidra 50 nM | Mean | 78.3 | 53.0 | 200.0 | 47616.6 |
| | STDER | 1.3 | 3.0 | 0.0 | 487.3 |
| PBMC + | 1st | 69.0 | 61.0 | 200.0 | 43901.3 |
| LPS(5 ug/ml) + | 2nd | 70.0 | 50.0 | 200.0 | 44314.5 |
| Xiidra 5 nM | Mean | 69.5 | 55.5 | 200.0 | 44107.9 |
| | STDER | 0.5 | 5.5 | 0.0 | 206.6 |
| PBMC + | 1st | 61.0 | 65.0 | 200.0 | 40481.0 |
| LPS(5 ug/ml) + | 2nd | 69.5 | 54.0 | 200.0 | 44108.3 |
| Xiidra 0.5 nM | Mean | 65.3 | 59.5 | 200.0 | 42294.6 |
| | STDER | 4.3 | 5.5 | 0.0 | 1813.6 |
| PBMC + | 1st | 66.5 | 50.0 | 200.0 | 42854.8 |
| LPS(5 ug/ml) + | 2nd | 67.0 | 59.0 | 200.0 | 43065.7 |
| Xiidra 0.05 nM | Mean | 66.8 | 54.5 | 200.0 | 42960.3 |
| | STDER | 0.3 | 4.5 | 0.0 | 105.4 |
| PBMC + | 1st | 61.5 | 60.0 | 200.0 | 40701.1 |
| LPS(5 ug/ml) + | 2nd | 61.0 | 71.0 | 200.0 | 40481.0 |
| Xiidra 0.005 nM | Mean | 61.3 | 65.5 | 200.0 | 40591.0 |
| | STDER | 0.3 | 5.5 | 0.0 | 110.1 |
| PBMC + | 1st | 46.5 | 66.0 | 200.0 | 33635.0 |
| LPS(5 ug/ml) + | 2nd | 49.0 | 75.0 | 200.0 | 34887.9 |
| YY101 500 nM | 3rd | 46.0 | 53.0 | 200.0 | 33380.1 |
| | Mean | 47.2 | 64.7 | 200.0 | 33967.7 |
| | STDER | 0.9 | 6.4 | 0.0 | 466.0 |
| PBMC + | 1st | 43.0 | 69.0 | 200.0 | 31818.4 |
| LPS(5 ug/ml) + | 2nd | 52.0 | 50.0 | 200.0 | 36347.5 |
| YY101 50 nM | 3rd | 45.0 | 67.0 | 200.0 | 32865.8 |
| | Mean | 46.7 | 62.0 | 200.0 | 33677.2 |
| | STDER | 2.7 | 6.0 | 0.0 | 1368.9 |
| PBMC + | 1st | 46.0 | 65.0 | 200.0 | 33380.1 |
| LPS(5 ug/ml) + | 2nd | 51.0 | 61.0 | 200.0 | 35866.0 |
| YY101 5 nM | 3rd | 48.0 | 61.0 | 200.0 | 34390.9 |
| | Mean | 48.3 | 62.3 | 200.0 | 34545.7 |
| | STDER | 1.5 | 1.3 | 0.0 | 721.8 |
| PBMC + | 1st | 51.5 | 50.0 | 200.0 | 36107.4 |
| LPS(5 ug/ml) + | 2nd | 46.0 | 57.0 | 200.0 | 33380.1 |
| YY101 0.5 nM | 3rd | 50.0 | 50.0 | 200.0 | 35379.6 |
| | Mean | 49.2 | 52.3 | 200.0 | 34955.7 |
| | STDER | 1.6 | 2.3 | 0.0 | 815.3 |
| PBMC + | 1st | 51.0 | 58.0 | 200.0 | 35866.0 |
| LPS(5 ug/ml) + | 2nd | 54.5 | 72.0 | 200.0 | 37530.4 |
| YY101 0.05 nM | 3rd | 51.0 | 60.0 | 200.0 | 35866.0 |
| | Mean | 52.2 | 63.3 | 200.0 | 36420.8 |
| | STDER | 1.2 | 4.4 | 0.0 | 554.8 |
| PBMC + | 1st | 49.5 | 50.0 | 200.0 | 35134.4 |
| LPS(5 ug/ml) + | 2nd | 46.0 | 56.0 | 200.0 | 33380.1 |
| YY101 0.005 nM | 3rd | 45.0 | 59.0 | 200.0 | 32865.8 |
| | Mean | 46.8 | 55.0 | 200.0 | 33793.5 |
| | STDER | 1.4 | 2.6 | 0.0 | 686.7 |
| PBMC + | 1st | 48.0 | 61.0 | 200.0 | 34390.9 |
| LPS(5 ug/ml) + | 2nd | 23.0 | 73.0 | 200.0 | 19244.9 |
| YDE011 500 nM | 3rd | 39.5 | 50.0 | 200.0 | 29918.7 |
| | Mean | 36.8 | 61.3 | 200.0 | 27851.5 |
| | STDER | 7.3 | 6.6 | 0.0 | 4492.8 |
| PBMC + | 1st | 41.5 | 50.0 | 200.0 | 31015.1 |
| LPS(5 ug/ml) + | 2nd | 45.0 | 57.0 | 200.0 | 32865.8 |
| YDE011 50 nM | 3rd | 25.5 | 50.0 | 200.0 | 21122.8 |
| | Mean | 37.3 | 52.3 | 200.0 | 28334.6 |
| | STDER | 6.0 | 2.3 | 0.0 | 3645.3 |
| PBMC + | 1st | 32.0 | 69.0 | 200.0 | 25497.0 |
| LPS(5 ug/ml) + | 2nd | 33.0 | 56.0 | 200.0 | 26119.3 |
| YDE011 5 nM | 3rd | 40.5 | 50.0 | 200.0 | 30470.7 |
| | Mean | 35.2 | 58.3 | 200.0 | 27362.3 |
| | STDER | 2.7 | 5.6 | 0.0 | 1564.5 |
| PBMC + | 1st | 42.0 | 71.0 | 200.0 | 31284.6 |
| LPS(5 ug/ml) + | 2nd | 39.0 | 50.0 | 200.0 | 29639.8 |
| YDE011 0.5 nM | 3rd | 40.5 | 58.0 | 200.0 | 30470.7 |
| | Mean | 40.5 | 59.7 | 200.0 | 30465.1 |
| | STDER | 0.9 | 6.1 | 0.0 | 474.8 |
| PBMC + | 1st | 48.0 | 59.0 | 200.0 | 34390.9 |
| LPS(5 ug/ml) + | 2nd | 45.0 | 50.0 | 200.0 | 32865.8 |
| YDE011 0.05 nM | 3rd | 50.5 | 70.0 | 200.0 | 35623.4 |
| | Mean | 47.8 | 59.7 | 200.0 | 34293.4 |
| | STDER | 1.6 | 5.8 | 0.0 | 797.5 |
| PBMC + | 1st | 43.0 | 51.0 | 200.0 | 31818.4 |
| LPS(5 ug/ml) + | 2nd | 37.5 | 50.0 | 200.0 | 28790.7 |
| YDE011 0.005 nM | 3rd | 42.0 | 69.0 | 200.0 | 31284.6 |
| | Mean | 40.8 | 56.7 | 200.0 | 30631.2 |
| | STDER | 1.7 | 6.2 | 0.0 | 933.1 |
| PBMC + | 1st | 37.0 | 50.0 | 200.0 | 28503.4 |
| LPS(5 ug/ml) + | 2nd | 33.5 | 54.0 | 200.0 | 26426.3 |
| YDE043 500 nM | 3rd | 38.5 | 64.0 | 200.0 | 29358.9 |
| | Mean | 36.3 | 56.0 | 200.0 | 28096.2 |
| | STDER | 1.5 | 4.2 | 0.0 | 870.7 |
| PBMC + | 1st | 46.0 | 55.0 | 200.0 | 33380.1 |
| LPS(5 ug/ml) + | 2nd | 46.0 | 58.0 | 200.0 | 33380.1 |
| YDE043 50 nM | 3rd | 37.0 | 70.0 | 200.0 | 28503.4 |
| | Mean | 43.0 | 61.0 | 200.0 | 31754.6 |
| | STDER | 3.0 | 4.6 | 0.0 | 1625.6 |
| PBMC + | 1st | 43.0 | 54.0 | 200.0 | 31818.4 |
| LPS(5 ug/ml) + | 2nd | 45.0 | 59.0 | 200.0 | 32865.8 |
| YDE043 5 nM | 3rd | 41.0 | 68.0 | 200.0 | 30743.9 |
| | Mean | 43.0 | 60.3 | 200.0 | 31809.4 |
| | STDER | 1.2 | 4.1 | 0.0 | 612.6 |
| PBMC + | 1st | 39.0 | 53.0 | 200.0 | 29639.8 |
| LPS(5 ug/ml) + | 2nd | 42.0 | 57.0 | 200.0 | 31284.6 |
| YDE043 0.5 nM | 3rd | 38.0 | 54.0 | 200.0 | 29075.9 |
| | Mean | 39.7 | 54.7 | 200.0 | 30000.1 |
| | STDER | 1.2 | 1.2 | 0.0 | 662.6 |
| PBMC + | 1st | 36.5 | 56.0 | 200.0 | 28213.9 |
| LPS(5 ug/ml) + | 2nd | 40.0 | 70.0 | 200.0 | 30195.7 |
| YDE043 0.05 nM | 3rd | 40.0 | 52.0 | 200.0 | 30195.7 |
| | Mean | 38.8 | 59.3 | 200.0 | 29535.1 |
| | STDER | 1.2 | 5.5 | 0.0 | 660.6 |
| PBMC + | 1st | 44.0 | 50.0 | 200.0 | 32345.4 |
| LPS(5 ug/ml) + | 2nd | 54.0 | 63.0 | 200.0 | 37296.1 |
| YDE043 0.005 nM | 3rd | 74.0 | 59.0 | 200.0 | 45939.1 |
| | Mean | 57.3 | 57.3 | 200.0 | 38526.9 |
| | STDER | 8.8 | 3.8 | 0.0 | 3972.1 |

TABLE 17
Bead name: CCL4/MIP-1 beta

| | | | | | Concentration |
|---|---|---|---|---|---|
| Sample | | MFI | Count | Dilution | (pg/ml) |
| Blank | 1st | 6.0 | 1.0 | 65.0 | <148.02 |
| | 2nd | 6.0 | 1.0 | 58.0 | <148.02 |
| | Mean | 6.0 | 1.0 | 61.5 | #DIV/0! |
| | STDER | 0.0 | 0.0 | 3.5 | #DIV/0! |
| PBMC + | 1st | 6.0 | 200.0 | 70.0 | <148.02 |
| 0.5% DMSO | 2nd | 6.0 | 200.0 | 70.0 | <148.02 |
| | 3rd | 6.0 | 200.0 | 94.0 | <148.02 |
| | Mean | 6.0 | 200.0 | 78.0 | #DIV/0! |
| | STDER | 0.0 | 0.0 | 8.0 | #DIV/0! |
| PBMC + | 1st | 20.0 | 200.0 | 73.0 | 36796.9 |
| LPS(5 ug/ml) | 2nd | 17.0 | 200.0 | 73.0 | 31897.6 |
| | 3rd | 18.0 | 200.0 | 104.0 | 33639.0 |
| | Mean | 18.3 | 200.0 | 83.3 | 34111.2 |
| | STDER | 0.9 | 0.0 | 10.3 | 1433.9 |
| PBMC + | 1st | 6.0 | 200.0 | 59.0 | <148.02 |
| YY101 500 nM | 2nd | 7.0 | 200.0 | 67.0 | <148.02 |
| | Mean | 6.5 | 200.0 | 63.0 | #DIV/0! |
| | STDER | 0.5 | 0.0 | 4.0 | #DIV/0! |
| PBMC + | 1st | 8.0 | 200.0 | 62.0 | <148.02 |
| YY101 50 nM | 2nd | 7.0 | 200.0 | 110.0 | <148.02 |
| | Mean | 7.5 | 200.0 | 86.0 | #DIV/0! |
| | STDER | 0.5 | 0.0 | 24.0 | #DIV/0! |
| PBMC + | 1st | 7.0 | 200.0 | 107.0 | <148.02 |
| YY101 5 nM | 2nd | 6.0 | 200.0 | 95.0 | <148.02 |
| | Mean | 6.5 | 200.0 | 101.0 | #DIV/0! |
| | STDER | 0.5 | 0.0 | 6.0 | #DIV/0! |
| PBMC + | 1st | 6.0 | 200.0 | 73.0 | <148.02 |
| YY101 0.5 nM | 2nd | 6.0 | 200.0 | 98.0 | <148.02 |
| | Mean | 6.0 | 200.0 | 85.5 | #DIV/0! |
| | STDER | 0.0 | 0.0 | 12.5 | #DIV/0! |
| PBMC + | 1st | 6.0 | 200.0 | 72.0 | <148.02 |
| YY101 0.05 uM | 2nd | 5.0 | 200.0 | 77.0 | <148.02 |
| | Mean | 5.5 | 200.0 | 74.5 | #DIV/0! |
| | STDER | 0.5 | 0.0 | 2.5 | #DIV/0! |
| PBMC + | 1st | 20.0 | 200.0 | 66.0 | 36796.9 |
| LPS(5 ug/ml) + | 2nd | 18.0 | 200.0 | 70.0 | 33639.0 |
| Xiidra 500 nM | Mean | 19.0 | 200.0 | 68.0 | 35218.0 |
| | STDER | 1.0 | 0.0 | 2.0 | 1578.9 |
| PBMC + | 1st | 15.0 | 200.0 | 63.0 | 27945.2 |
| LPS(5 ug/ml) + | 2nd | 16.0 | 200.0 | 68.0 | 30013.2 |
| Xiidra 50 nM | Mean | 15.5 | 200.0 | 65.5 | 28979.2 |
| | STDER | 0.5 | 0.0 | 2.5 | 1034.0 |
| PBMC + | 1st | 16.0 | 200.0 | 70.0 | 30013.2 |
| LPS(5 ug/ml) + | 2nd | 16.0 | 200.0 | 90.0 | 30013.2 |
| Xiidra 5 nM | Mean | 16.0 | 200.0 | 80.0 | 30013.2 |
| | STDER | 0.0 | 0.0 | 10.0 | 0.0 |
| PBMC + | 1st | 16.0 | 200.0 | 81.0 | 30013.2 |
| LPS(5 ug/ml) + | 2nd | 17.0 | 200.0 | 62.0 | 31897.6 |
| Xiidra 0.5 nM | Mean | 16.5 | 200.0 | 71.5 | 30955.4 |
| | STDER | 0.5 | 0.0 | 9.5 | 942.2 |
| PBMC + | 1st | 15.5 | 200.0 | 88.0 | 29005.5 |
| LPS(5 ug/ml) + | 2nd | 17.0 | 200.0 | 97.0 | 31897.6 |
| Xiidra 0.05 nM | Mean | 16.3 | 200.0 | 92.5 | 30451.5 |
| | STDER | 0.8 | 0.0 | 4.5 | 1446.1 |
| PBMC + | 1st | 16.0 | 200.0 | 85.0 | 30013.2 |
| LPS(5 ug/ml) + | 2nd | 14.0 | 200.0 | 62.0 | 25629.7 |
| Xiidra 0.005 nM | Mean | 15.0 | 200.0 | 73.5 | 27821.4 |
| | STDER | 1.0 | 0.0 | 11.5 | 2191.8 |
| PBMC + | 1st | 11.0 | 200.0 | 60.0 | 15257.5 |
| LPS(5 ug/ml) + | 2nd | 12.0 | 200.0 | 71.0 | 19701.2 |
| YY101 500 nM | 3rd | 12.0 | 200.0 | 72.0 | 19701.2 |
| | Mean | 11.7 | 200.0 | 67.7 | 18220.0 |
| | STDER | 0.3 | 0.0 | 3.8 | 1481.3 |
| PBMC + | 1st | 11.0 | 200.0 | 65.0 | 15257.5 |
| LPS(5 ug/ml) + | 2nd | 12.0 | 200.0 | 104.0 | 19701.2 |
| YY101 50 nM | 3rd | 11.0 | 200.0 | 90.0 | 15257.5 |
| | Mean | 11.3 | 200.0 | 86.3 | 16738.7 |
| | STDER | 0.3 | 0.0 | 11.4 | 1481.3 |
| PBMC + | 1st | 11.5 | 200.0 | 76.0 | 17703.4 |
| LPS(5 ug/ml) + | 2nd | 11.0 | 200.0 | 92.0 | 15257.5 |
| YY101 5 nM | 3rd | 11.0 | 200.0 | 79.0 | 15257.5 |
| | Mean | 11.2 | 200.0 | 82.3 | 16072.8 |
| | STDER | 0.2 | 0.0 | 4.9 | 815.3 |
| PBMC + | 1st | 11.5 | 200.0 | 98.0 | 17703.4 |
| LPS(5 ug/ml) + | 2nd | 14.0 | 200.0 | 85.0 | 25629.7 |
| YY101 0.5 nM | 3rd | 12.0 | 200.0 | 73.0 | 19701.2 |
| | Mean | 12.5 | 200.0 | 85.3 | 21011.4 |
| | STDER | 0.8 | 0.0 | 7.2 | 2380.0 |
| PBMC + | 1st | 14.0 | 200.0 | 50.0 | 25629.7 |
| LPS(5 ug/ml) + | 2nd | 11.5 | 200.0 | 74.0 | 17703.4 |
| YY101 0.05 nM | 3rd | 12.0 | 200.0 | 83.0 | 19701.2 |
| | Mean | 12.5 | 200.0 | 69.0 | 21011.4 |
| | STDER | 0.8 | 0.0 | 9.8 | 2380.0 |
| PBMC + | 1st | 14.0 | 200.0 | 84.0 | 25629.7 |
| LPS(5 ug/ml) + | 2nd | 12.0 | 200.0 | 70.0 | 19701.2 |
| YY101 0.005 nM | 3rd | 12.0 | 200.0 | 70.0 | 19701.2 |
| | Mean | 12.7 | 200.0 | 74.7 | 21677.4 |
| | STDER | 0.7 | 0.0 | 4.7 | 1976.1 |
| PBMC + | 1st | 12.0 | 200.0 | 61.0 | 19701.2 |
| LPS(5 ug/ml) + | 2nd | 10.0 | 200.0 | 89.0 | 1874.9 |
| YDE011 500 nM | 3rd | 12.0 | 200.0 | 61.0 | 19701.2 |
| | Mean | 11.3 | 200.0 | 70.3 | 13759.1 |
| | STDER | 0.7 | 0.0 | 9.3 | 5942.1 |
| PBMC + | 1st | 13.0 | 200.0 | 77.0 | 22956.0 |
| LPS(5 ug/ml) + | 2nd | 14.0 | 200.0 | 102.0 | 25629.7 |
| YDE011 50 nM | 3rd | 13.0 | 200.0 | 74.0 | 22956.0 |
| | Mean | 13.3 | 200.0 | 84.3 | 23847.2 |
| | STDER | 0.3 | 0.0 | 8.9 | 891.2 |
| PBMC + | 1st | 11.0 | 200.0 | 64.0 | 15257.5 |
| LPS(5 ug/ml) + | 2nd | 12.0 | 200.0 | 95.0 | 19701.2 |
| YDE011 5 nM | 3rd | 11.0 | 200.0 | 103.0 | 15257.5 |
| | Mean | 11.3 | 200.0 | 87.3 | 16738.7 |
| | STDER | 0.3 | 0.0 | 11.9 | 1481.3 |
| PBMC + | 1st | 13.0 | 200.0 | 95.0 | 22956.0 |
| LPS(5 ug/ml) + | 2nd | 13.0 | 200.0 | 75.0 | 22956.0 |
| YDE011 0.5 nM | 3rd | 13.0 | 200.0 | 76.0 | 22956.0 |
| | Mean | 13.0 | 200.0 | 82.0 | 22956.0 |
| | STDER | 0.0 | 0.0 | 6.5 | 0.0 |
| PBMC + | 1st | 13.0 | 200.0 | 53.0 | 22956.0 |
| LPS(5 ug/ml) + | 2nd | 13.0 | 200.0 | 62.0 | 22956.0 |
| YDE011 0.05 nM | 3rd | 13.0 | 200.0 | 92.0 | 22956.0 |
| | Mean | 13.0 | 200.0 | 69.0 | 22956.0 |
| | STDER | 0.0 | 0.0 | 11.8 | 0.0 |
| PBMC + | 1st | 12.0 | 200.0 | 87.0 | 19701.2 |
| LPS(5 ug/ml) + | 2nd | 13.0 | 200.0 | 97.0 | 22956.0 |
| YDE011 0.005 nM | 3rd | 12.0 | 200.0 | 74.0 | 19701.2 |
| | Mean | 12.3 | 200.0 | 86.0 | 20786.2 |
| | STDER | 0.3 | 0.0 | 6.7 | 1084.9 |
| PBMC + | 1st | 14.0 | 200.0 | 79.0 | 25629.7 |
| LPS(5 ug/ml) + | 2nd | 13.0 | 200.0 | 65.0 | 22956.0 |
| YDE043 500 nM | 3rd | 12.0 | 200.0 | 74.0 | 19701.2 |
| | Mean | 13.0 | 200.0 | 72.7 | 22762.3 |
| | STDER | 0.6 | 0.0 | 4.1 | 1714.1 |
| PBMC + | 1st | 13.0 | 200.0 | 79.0 | 22956.0 |
| LPS(5 ug/ml) + | 2nd | 13.0 | 200.0 | 76.0 | 22956.0 |
| YDE043 50 nM | 3rd | 11.5 | 200.0 | 96.0 | 17703.4 |
| | Mean | 12.5 | 200.0 | 83.7 | 21205.1 |
| | STDER | 0.5 | 0.0 | 6.2 | 1750.9 |
| PBMC + | 1st | 12.0 | 200.0 | 77.0 | 19701.2 |
| LPS(5 ug/ml) + | 2nd | 13.0 | 200.0 | 73.0 | 22956.0 |
| YDE043 5 nM | 3rd | 12.0 | 200.0 | 85.0 | 19701.2 |
| | Mean | 12.3 | 200.0 | 78.3 | 20786.2 |
| | STDER | 0.3 | 0.0 | 3.5 | 1084.9 |
| PBMC + | 1st | 13.0 | 200.0 | 80.0 | 22956.0 |
| LPS(5 ug/ml) + | 2nd | 13.0 | 200.0 | 74.0 | 22956.0 |
| YDE043 0.5 nM | 3rd | 12.0 | 200.0 | 57.0 | 19701.2 |
| | Mean | 12.7 | 200.0 | 70.3 | 21871.1 |
| | STDER | 0.3 | 0.0 | 6.9 | 1084.9 |
| PBMC + | 1st | 12.0 | 200.0 | 92.0 | 19701.2 |
| LPS(5 ug/ml) + | 2nd | 12.0 | 200.0 | 96.0 | 19701.2 |
| YDE043 0.05 nM | 3rd | 12.0 | 200.0 | 78.0 | 19701.2 |
| | Mean | 12.0 | 200.0 | 88.7 | 19701.2 |
| | STDER | 0.0 | 0.0 | 5.5 | 0.0 |
| PBMC + | 1st | 13.0 | 200.0 | 69.0 | 22956.0 |
| LPS(5 ug/ml) + | 2nd | 16.0 | 200.0 | 73.0 | 30013.2 |
| YDE043 0.005 nM | 3rd | 12.0 | 200.0 | 78.0 | 19701.2 |
| | Mean | 13.7 | 200.0 | 73.3 | 24223.5 |
| | STDER | 1.2 | 0.0 | 2.6 | 3043.5 |

TABLE 18
Bead name: IL-1 alpha/IL-1F1

| | | | | | Concentration |
|---|---|---|---|---|---|
| Sample | | MFI | Count | Dilution | (pg/ml) |
| Blank | 1st | 20.0 | 57.0 | 1.0 | 0.8 |
| | 2nd | 16.0 | 66.0 | 1.0 | 0.0 |
| | Mean | 18.0 | 61.5 | 1.0 | 0.4 |
| | STDER | 2.0 | 4.5 | 0.0 | 0.4 |
| PBMC + | 1st | 18.0 | 63.0 | 200.0 | 84.2 |
| 0.5% DMSO | 2nd | 18.5 | 62.0 | 200.0 | 102.5 |
| | 3rd | 18.0 | 89.0 | 200.0 | 84.2 |
| | Mean | 18.2 | 71.3 | 200.0 | 90.3 |
| | STDER | 0.2 | 8.8 | 0.0 | 6.1 |
| PBMC + | 1st | 39.0 | 64.0 | 200.0 | 740.9 |
| LPS(5 ug/ml) | 2nd | 31.0 | 63.0 | 200.0 | 505.6 |
| | 3rd | 31.0 | 73.0 | 200.0 | 505.6 |
| | Mean | 33.7 | 66.7 | 200.0 | 584.0 |
| | STDER | 2.7 | 3.2 | 0.0 | 78.4 |
| PBMC + | 1st | 18.0 | 57.0 | 200.0 | 84.2 |
| YY101 500 nM | 2nd | 18.0 | 81.0 | 200.0 | 84.2 |
| | Mean | 18.0 | 69.0 | 200.0 | 84.2 |
| | STDER | 0.0 | 12.0 | 0.0 | 0.0 |
| PBMC + | 1st | 19.5 | 64.0 | 200.0 | 138.1 |
| YY101 50 nM | 2nd | 18.0 | 53.0 | 200.0 | 84.2 |
| | Mean | 18.8 | 58.5 | 200.0 | 111.1 |
| | STDER | 0.8 | 5.5 | 0.0 | 27.0 |
| PBMC + | 1st | 20.0 | 75.0 | 200.0 | 155.5 |
| YY101 5 nM | 2nd | 20.0 | 60.0 | 200.0 | 155.5 |
| | Mean | 20.0 | 67.5 | 200.0 | 155.5 |
| | STDER | 0.0 | 7.5 | 0.0 | 0.0 |
| PBMC + | 1st | 20.0 | 70.0 | 200.0 | 155.5 |
| YY101 0.5 nM | 2nd | 20.0 | 66.0 | 200.0 | 155.5 |
| | Mean | 20.0 | 68.0 | 200.0 | 155.5 |
| | STDER | 0.0 | 2.0 | 0.0 | 0.0 |
| PBMC + | 1st | 17.0 | 74.0 | 200.0 | 45.8 |
| YY101 0.05 uM | 2nd | 19.5 | 54.0 | 200.0 | 138.1 |
| | Mean | 18.3 | 64.0 | 200.0 | 92.0 |
| | STDER | 1.3 | 10.0 | 0.0 | 46.2 |
| PBMC + | 1st | 38.0 | 73.0 | 200.0 | 712.0 |
| LPS(5 ug/ml) + | 2nd | 37.0 | 50.0 | 200.0 | 683.0 |
| Xiidra 500 nM | Mean | 37.5 | 61.5 | 200.0 | 697.5 |
| | STDER | 0.5 | 11.5 | 0.0 | 14.5 |
| PBMC + | 1st | 37.5 | 74.0 | 200.0 | 697.5 |
| LPS(5 ug/ml) + | 2nd | 40.0 | 82.0 | 200.0 | 769.6 |
| Xiidra 50 nM | Mean | 38.8 | 78.0 | 200.0 | 733.6 |
| | STDER | 1.3 | 4.0 | 0.0 | 36.1 |
| PBMC + | 1st | 32.5 | 66.0 | 200.0 | 550.5 |
| LPS(5 ug/ml) + | 2nd | 35.5 | 66.0 | 200.0 | 639.2 |
| Xiidra 5 nM | Mean | 34.0 | 66.0 | 200.0 | 594.9 |
| | STDER | 1.5 | 0.0 | 0.0 | 44.3 |
| PBMC + | 1st | 28.5 | 64.0 | 200.0 | 429.6 |
| LPS(5 ug/ml) + | 2nd | 32.0 | 61.0 | 200.0 | 535.6 |
| Xiidra 0.5 nM | Mean | 30.3 | 62.5 | 200.0 | 482.6 |
| | STDER | 1.8 | 1.5 | 0.0 | 53.0 |
| PBMC + | 1st | 29.0 | 89.0 | 200.0 | 444.9 |
| LPS(5 ug/ml) + | 2nd | 30.0 | 70.0 | 200.0 | 475.3 |
| Xiidra 0.05 nM | Mean | 29.5 | 79.5 | 200.0 | 460.1 |
| | STDER | 0.5 | 9.5 | 0.0 | 15.2 |
| PBMC + | 1st | 30.0 | 66.0 | 200.0 | 475.3 |
| LPS(5 ug/ml) + | 2nd | 27.0 | 62.0 | 200.0 | 383.2 |
| Xiidra 0.005 nM | Mean | 28.5 | 64.0 | 200.0 | 429.3 |
| | STDER | 1.5 | 2.0 | 0.0 | 46.1 |
| PBMC + | 1st | 29.5 | 56.0 | 200.0 | 460.1 |
| LPS(5 ug/ml) + | 2nd | 31.0 | 74.0 | 200.0 | 505.6 |
| YY101 500nM | 3rd | 27.0 | 71.0 | 200.0 | 383.2 |
| | Mean | 29.2 | 67.0 | 200.0 | 449.6 |
| | STDER | 1.2 | 5.6 | 0.0 | 35.7 |
| PBMC + | 1st | 27.0 | 69.0 | 200.0 | 383.2 |
| LPS(5 ug/ml) + | 2nd | 27.5 | 58.0 | 200.0 | 398.7 |
| YY101 50 nM | 3rd | 31.0 | 69.0 | 200.0 | 505.6 |
| | Mean | 28.5 | 65.3 | 200.0 | 429.2 |
| | STDER | 1.3 | 3.7 | 0.0 | 38.5 |
| PBMC + | 1st | 29.0 | 64.0 | 200.0 | 444.9 |
| LPS(5 ug/ml) + | 2nd | 30.0 | 64.0 | 200.0 | 475.3 |
| YY101 5 nM | 3rd | 32.0 | 57.0 | 200.0 | 535.6 |
| | Mean | 30.3 | 61.7 | 200.0 | 485.3 |
| | STDER | 0.9 | 2.3 | 0.0 | 26.7 |
| PBMC + | 1st | 29.5 | 72.0 | 200.0 | 460.1 |
| LPS(5 ug/ml) + | 2nd | 29.0 | 80.0 | 200.0 | 444.9 |
| YY101 0.5 nM | 3rd | 29.5 | 60.0 | 200.0 | 460.1 |
| | Mean | 29.3 | 70.7 | 200.0 | 455.0 |
| | STDER | 0.2 | 5.8 | 0.0 | 5.1 |
| PBMC + | 1st | 28.0 | 74.0 | 200.0 | 414.2 |
| LPS(5 ug/ml) + | 2nd | 34.5 | 66.0 | 200.0 | 609.8 |
| YY101 0.05 nM | 3rd | 28.0 | 61.0 | 200.0 | 414.2 |
| | Mean | 30.2 | 67.0 | 200.0 | 479.4 |
| | STDER | 2.2 | 3.8 | 0.0 | 65.2 |
| PBMC + | 1st | 28.0 | 58.0 | 200.0 | 414.2 |
| LPS(5 ug/ml) + | 2nd | 27.0 | 55.0 | 200.0 | 383.2 |
| YY101 0.005 nM | 3rd | 30.0 | 61.0 | 200.0 | 475.3 |
| | Mean | 28.3 | 58.0 | 200.0 | 424.2 |
| | STDER | 0.9 | 1.7 | 0.0 | 27.1 |
| PBMC + | 1st | 32.0 | 60.0 | 200.0 | 535.6 |
| LPS(5 ug/ml) + | 2nd | 16.0 | 67.0 | 200.0 | 1.9 |
| YDE011 500 nM | 3rd | 24.0 | 56.0 | 200.0 | 288.4 |
| | Mean | 24.0 | 61.0 | 200.0 | 275.3 |
| | STDER | 4.6 | 3.2 | 0.0 | 154.2 |
| PBMC + | 1st | 28.5 | 74.0 | 200.0 | 429.6 |
| LPS(5 ug/ml) + | 2nd | 25.0 | 59.0 | 200.0 | 320.3 |
| YDE011 50 nM | 3rd | 18.0 | 63.0 | 200.0 | 84.2 |
| | Mean | 23.8 | 65.3 | 200.0 | 278.0 |
| | STDER | 3.1 | 4.5 | 0.0 | 101.9 |
| PBMC + | 1st | 23.0 | 75.0 | 200.0 | 256.0 |
| LPS(5 ug/ml) + | 2nd | 24.0 | 74.0 | 200.0 | 288.4 |
| YDE011 5 nM | 3rd | 28.5 | 70.0 | 200.0 | 429.6 |
| | Mean | 25.2 | 73.0 | 200.0 | 324.6 |
| | STDER | 1.7 | 1.5 | 0.0 | 53.3 |
| PBMC + | 1st | 28.5 | 84.0 | 200.0 | 429.6 |
| LPS(5 ug/ml) + | 2nd | 27.0 | 86.0 | 200.0 | 383.2 |
| YDE011 0.5 nM | 3rd | 27.5 | 70.0 | 200.0 | 398.7 |
| | Mean | 27.7 | 80.0 | 200.0 | 403.8 |
| | STDER | 0.4 | 5.0 | 0.0 | 13.6 |
| PBMC + | 1st | 32.0 | 74.0 | 200.0 | 535.6 |
| LPS(5 ug/ml) + | 2nd | 32.5 | 70.0 | 200.0 | 550.5 |
| YDE011 0.05 nM | 3rd | 29.0 | 66.0 | 200.0 | 444.9 |
| | Mean | 31.2 | 70.0 | 200.0 | 510.3 |
| | STDER | 1.1 | 2.3 | 0.0 | 33.0 |
| PBMC + | 1st | 34.0 | 60.0 | 200.0 | 595.1 |
| LPS(5 ug/ml) + | 2nd | 29.5 | 68.0 | 200.0 | 460.1 |
| YDE011 0.005 nM | 3rd | 30.0 | 86.0 | 200.0 | 475.3 |
| | Mean | 31.2 | 71.3 | 200.0 | 510.2 |
| | STDER | 1.4 | 7.7 | 0.0 | 42.7 |
| PBMC + | 1st | 30.0 | 67.0 | 200.0 | 475.3 |
| LPS(5 ug/ml) + | 2nd | 28.0 | 55.0 | 200.0 | 414.2 |
| YDE043 500 nM | 3rd | 27.0 | 60.0 | 200.0 | 383.2 |
| | Mean | 28.3 | 60.7 | 200.0 | 424.2 |
| | STDER | 0.9 | 3.5 | 0.0 | 27.1 |
| PBMC + | 1st | 30.0 | 76.0 | 200.0 | 475.3 |
| LPS(5 ug/ml) + | 2nd | 29.0 | 62.0 | 200.0 | 444.9 |
| YDE043 50 nM | 3rd | 27.0 | 83.0 | 200.0 | 383.2 |
| | Mean | 28.7 | 73.7 | 200.0 | 434.5 |
| | STDER | 0.9 | 6.2 | 0.0 | 27.1 |
| PBMC + | 1st | 31.0 | 64.0 | 200.0 | 505.6 |
| LPS(5 ug/ml) + | 2nd | 29.0 | 54.0 | 200.0 | 444.9 |
| YDE043 5 nM | 3rd | 29.0 | 70.0 | 200.0 | 444.9 |
| | Mean | 29.7 | 62.7 | 200.0 | 465.1 |
| | STDER | 0.7 | 4.7 | 0.0 | 20.2 |
| PBMC + | 1st | 26.0 | 57.0 | 200.0 | 351.9 |
| LPS(5 ug/ml) + | 2nd | 26.5 | 50.0 | 200.0 | 367.6 |
| YDE043 0.5 nM | 3rd | 25.5 | 64.0 | 200.0 | 336.2 |
| | Mean | 26.0 | 57.0 | 200.0 | 351.9 |
| | STDER | 0.3 | 4.0 | 0.0 | 9.1 |
| PBMC + | 1st | 26.0 | 74.0 | 200.0 | 351.9 |
| LPS(5 ug/ml) + | 2nd | 25.0 | 64.0 | 200.0 | 320.3 |
| YDE043 0.05 nM | 3rd | 25.0 | 78.0 | 200.0 | 320.3 |
| | Mean | 25.3 | 72.0 | 200.0 | 330.9 |
| | STDER | 0.3 | 4.2 | 0.0 | 10.5 |
| PBMC + | 1st | 27.0 | 71.0 | 200.0 | 383.2 |
| LPS(5 ug/ml) + | 2nd | 30.5 | 50.0 | 200.0 | 490.5 |
| YDE043 0.005 nM | 3rd | 34.0 | 60.0 | 200.0 | 595.1 |
| | Mean | 30.5 | 60.3 | 200.0 | 489.6 |
| | STDER | 2.0 | 6.1 | 0.0 | 61.2 |

TABLE 19
Bead name: IL-1 beta/IL-1F2

| | | | | | Concen- |
|---|---|---|---|---|---|
| | | | | Dilu- | tration |
| Sample | | MFI | Count | tion | (pg/ml) |
| Blank | 1st | 14.0 | 55.0 | 1.0 | 2.1 |
| | 2nd | 13.0 | 69.0 | 1.0 | 1.8 |
| | Mean | 13.5 | 62.0 | 1.0 | 2.0 |
| | STDER | 0.5 | 7.0 | 0.0 | 0.1 |
| PBMC + 0.5% DMSO | 1st | 12.0 | 71.0 | 200.0 | 304.1 |
| | 2nd | 12.0 | 79.0 | 200.0 | 304.1 |
| | 3rd | 14.0 | 84.0 | 200.0 | 420.1 |
| | Mean | 12.7 | 78.0 | 200.0 | 342.8 |
| | STDER | 0.7 | 3.8 | 0.0 | 38.7 |
| PBMC + LPS(5 ug/ml) | 1st | 141.5 | 74.0 | 200.0 | 7972.5 |
| | 2nd | 117.0 | 69.0 | 200.0 | 6511.1 |
| | 3rd | 106.0 | 72.0 | 200.0 | 5855.9 |
| | Mean | 121.5 | 71.7 | 200.0 | 6779.8 |
| | STDER | 10.5 | 1.5 | 0.0 | 625.6 |
| PBMC + YY101 500 nM | 1st | 14.0 | 100.0 | 200.0 | 420.1 |
| | 2nd | 14.0 | 101.0 | 200.0 | 420.1 |
| | Mean | 14.0 | 100.5 | 200.0 | 420.1 |
| | STDER | 0.0 | 0.5 | 0.0 | 0.0 |
| PBMC + YY101 50 nM | 1st | 13.0 | 80.0 | 200.0 | 362.0 |
| | 2nd | 13.0 | 78.0 | 200.0 | 362.0 |
| | Mean | 13.0 | 79.0 | 200.0 | 362.0 |
| | STDER | 0.0 | 1.0 | 0.0 | 0.0 |
| PBMC + YY101 5 nM | 1st | 13.0 | 80.0 | 200.0 | 362.0 |
| | 2nd | 14.0 | 77.0 | 200.0 | 420.1 |
| | Mean | 13.5 | 78.5 | 200.0 | 391.1 |
| | STDER | 0.5 | 1.5 | 0.0 | 29.0 |
| PBMC + YY101 0.5 nM | 1st | 13.0 | 63.0 | 200.0 | 362.0 |
| | 2nd | 14.0 | 84.0 | 200.0 | 420.1 |
| | Mean | 13.5 | 73.5 | 200.0 | 391.1 |
| | STDER | 0.5 | 10.5 | 0.0 | 29.0 |
| PBMC + YY101 0.05 uM | 1st | 14.0 | 89.0 | 200.0 | 420.1 |
| | 2nd | 14.0 | 67.0 | 200.0 | 420.1 |
| | Mean | 14.0 | 78.0 | 200.0 | 420.1 |
| | STDER | 0.0 | 11.0 | 0.0 | 0.0 |
| PBMC + LPS(5 ug/ml) + | 1st | 144.5 | 96.0 | 200.0 | 8151.7 |
| Xiidra 500 nM | 2nd | 149.0 | 69.0 | 200.0 | 8420.4 |
| | Mean | 146.8 | 82.5 | 200.0 | 8286.0 |
| | STDER | 2.3 | 13.5 | 0.0 | 134.4 |
| PBMC + LPS(5 ug/ml) + | 1st | 141.0 | 71.0 | 200.0 | 7942.7 |
| Xiidra 50 nM | 2nd | 152.0 | 84.0 | 200.0 | 8599.7 |
| | Mean | 146.5 | 77.5 | 200.0 | 8271.2 |
| | STDER | 5.5 | 6.5 | 0.0 | 328.5 |
| PBMC + LPS(5 ug/ml) + | 1st | 125.0 | 73.0 | 200.0 | 6988.0 |
| Xiidra 5 nM | 2nd | 142.0 | 62.0 | 200.0 | 8002.4 |
| | Mean | 133.5 | 67.5 | 200.0 | 7495.2 |
| | STDER | 8.5 | 5.5 | 0.0 | 507.2 |
| PBMC + LPS(5 ug/ml) + | 1st | 108.0 | 99.0 | 200.0 | 5975.0 |
| Xiidra 0.5 nM | 2nd | 124.0 | 78.0 | 200.0 | 6928.3 |
| | Mean | 116.0 | 88.5 | 200.0 | 6451.7 |
| | STDER | 8.0 | 10.5 | 0.0 | 476.7 |
| PBMC + LPS(5 ug/ml) + | 1st | 116.5 | 82.0 | 200.0 | 6481.3 |
| Xiidra 0.05 nM | 2nd | 113.0 | 95.0 | 200.0 | 6272.8 |
| | Mean | 114.8 | 88.5 | 200.0 | 6377.0 |
| | STDER | 1.8 | 6.5 | 0.0 | 104.3 |
| PBMC + LPS(5 ug/ml) + | 1st | 118.0 | 71.0 | 200.0 | 6570.7 |
| Xiidra 0.005 nM | 2nd | 104.0 | 67.0 | 200.0 | 5736.8 |
| | Mean | 111.0 | 69.0 | 200.0 | 6153.8 |
| | STDER | 7.0 | 2.0 | 0.0 | 416.9 |
| PBMC + LPS(5 ug/ml) + | 1st | 96.0 | 73.0 | 200.0 | 5260.9 |
| YY101 500 nM | 2nd | 107.0 | 95.0 | 200.0 | 5915.4 |
| | 3rd | 101.0 | 81.0 | 200.0 | 5558.3 |
| | Mean | 101.3 | 83.0 | 200.0 | 5578.2 |
| | STDER | 3.2 | 6.4 | 0.0 | 189.2 |
| PBMC + LPS(5 ug/ml) + | 1st | 95.0 | 76.0 | 200.0 | 5201.4 |
| YY101 50 nM | 2nd | 95.0 | 67.0 | 200.0 | 5201.4 |
| | 3rd | 115.5 | 66.0 | 200.0 | 6421.7 |
| | Mean | 101.8 | 69.7 | 200.0 | 5608.2 |
| | STDER | 6.8 | 3.2 | 0.0 | 406.8 |
| PBMC + LPS(5 ug/ml) + | 1st | 100.0 | 78.0 | 200.0 | 5498.8 |
| YY101 5 nM | 2nd | 101.0 | 63.0 | 200.0 | 5558.3 |
| | 3rd | 118.0 | 75.0 | 200.0 | 6570.7 |
| | Mean | 106.3 | 72.0 | 200.0 | 5875.9 |
| | STDER | 5.8 | 4.6 | 0.0 | 347.8 |
| PBMC + LPS(5 ug/ml) + | 1st | 104.0 | 85.0 | 200.0 | 5736.8 |
| YY101 0.5 nM | 2nd | 108.0 | 86.0 | 200.0 | 5975.0 |
| | 3rd | 115.5 | 74.0 | 200.0 | 6421.7 |
| | Mean | 109.2 | 81.7 | 200.0 | 6044.5 |
| | STDER | 3.4 | 3.8 | 0.0 | 200.7 |
| PBMC + LPS(5 ug/ml) + | 1st | 104.0 | 85.0 | 200.0 | 5736.8 |
| YY101 0.05 nM | 2nd | 117.0 | 74.0 | 200.0 | 6511.1 |
| | 3rd | 104.5 | 78.0 | 200.0 | 5766.6 |
| | Mean | 108.5 | 79.0 | 200.0 | 6004.8 |
| | STDER | 4.3 | 3.2 | 0.0 | 253.3 |
| PBMC + LPS(5 ug/ml) +M | 1st | 108.0 | 71.0 | 200.0 | 5975.0 |
| YY101 0.005 nM | 2nd | 108.5 | 64.0 | 200.0 | 6004.7 |
| | 3rd | 99.0 | 58.0 | 200.0 | 5439.3 |
| | Mean | 105.2 | 64.3 | 200.0 | 5806.3 |
| | STDER | 3.1 | 3.8 | 0.0 | 183.7 |
| PBMC + LPS(5 ug/ml) + | 1st | 105.0 | 81.0 | 200.0 | 5796.4 |
| YDE011 500 nM | 2nd | 52.5 | 74.0 | 200.0 | 2681.1 |
| | 3rd | 80.0 | 70.0 | 200.0 | 4310.2 |
| | Mean | 79.2 | 75.0 | 200.0 | 4262.6 |
| | STDER | 15.2 | 3.2 | 0.0 | 899.6 |
| PBMC + LPS(5 ug/ml) + | 1st | 101.0 | 81.0 | 200.0 | 5558.3 |
| YDE011 50 nM | 2nd | 102.0 | 81.0 | 200.0 | 5617.8 |
| | 3rd | 61.0 | 63.0 | 200.0 | 3183.9 |
| | Mean | 88.0 | 75.0 | 200.0 | 4786.7 |
| | STDER | 13.5 | 6.0 | 0.0 | 801.6 |
| PBMC + LPS(5 ug/ml) + | 1st | 67.0 | 74.0 | 200.0 | 3539.2 |
| YDE011 5 nM | 2nd | 79.0 | 61.0 | 200.0 | 4250.8 |
| | 3rd | 96.0 | 70.0 | 200.0 | 5260.9 |
| | Mean | 80.7 | 68.3 | 200.0 | 4350.3 |
| | STDER | 8.4 | 3.8 | 0.0 | 499.5 |
| PBMC + LPS(5 ug/ml) + | 1st | 89.0 | 89.0 | 200.0 | 4844.7 |
| YDE011 0.5 nM | 2nd | 91.0 | 79.0 | 200.0 | 4963.6 |
| | 3rd | 88.0 | 79.0 | 200.0 | 4785.3 |
| | Mean | 89.3 | 82.3 | 200.0 | 4864.5 |
| | STDER | 0.9 | 3.3 | 0.0 | 52.4 |
| PBMC + LPS(5 ug/ml) + | 1st | 105.0 | 82.0 | 200.0 | 5796.4 |
| YDE011 0.05 nM | 2nd | 110.0 | 62.0 | 200.0 | 6094.1 |
| | 3rd | 102.0 | 83.0 | 200.0 | 5617.8 |
| | Mean | 105.7 | 75.7 | 200.0 | 5836.1 |
| | STDER | 2.3 | 6.8 | 0.0 | 138.9 |
| PBMC + LPS(5 ug/ml) + | 1st | 97.5 | 60.0 | 200.0 | 5350.1 |
| YDE011 0.005 nM | 2nd | 92.0 | 86.0 | 200.0 | 5023.0 |
| | 3rd | 94.0 | 85.0 | 200.0 | 5141.9 |
| | Mean | 94.5 | 77.0 | 200.0 | 5171.7 |
| | STDER | 1.6 | 8.5 | 0.0 | 95.6 |
| PBMC + LPS(5 ug/ml) + | 1st | 82.0 | 70.0 | 200.0 | 4428.9 |
| YDE043 500 nM | 2nd | 78.0 | 86.0 | 200.0 | 4191.5 |
| | 3rd | 90.0 | 76.0 | 200.0 | 4904.1 |
| | Mean | 83.3 | 77.3 | 200.0 | 4508.2 |
| | STDER | 3.5 | 4.7 | 0.0 | 209.5 |
| PBMC + LPS(5 ug/ml) + | 1st | 101.5 | 78.0 | 200.0 | 5588.1 |
| YDE043 50 nM | 2nd | 99.5 | 78.0 | 200.0 | 5469.1 |
| | 3rd | 88.0 | 88.0 | 200.0 | 4785.3 |
| | Mean | 96.3 | 81.3 | 200.0 | 5280.8 |
| | STDER | 4.2 | 3.3 | 0.0 | 250.1 |
| PBMC + LPS(5 ug/ml) + | 1st | 98.0 | 59.0 | 200.0 | 5379.8 |
| YDE043 5 nM | 2nd | 101.0 | 74.0 | 200.0 | 5558.3 |
| | 3rd | 95.0 | 86.0 | 200.0 | 5201.4 |
| | Mean | 98.0 | 73.0 | 200.0 | 5379.8 |
| | STDER | 1.7 | 7.8 | 0.0 | 103.0 |
| PBMC + LPS(5 ug/ml) + | 1st | 91.0 | 71.0 | 200.0 | 4963.6 |
| YDE043 0.5 nM | 2nd | 88.0 | 83.0 | 200.0 | 4785.3 |
| | 3rd | 82.0 | 65.0 | 200.0 | 4428.9 |
| | Mean | 87.0 | 73.0 | 200.0 | 4725.9 |
| | STDER | 2.6 | 5.3 | 0.0 | 157.2 |
| PBMC + LPS(5 ug/ml) + | 1st | 83.0 | 73.0 | 200.0 | 4488.3 |
| YDE043 0.05 nM | 2nd | 82.0 | 71.0 | 200.0 | 4428.9 |
| | 3rd | 82.5 | 80.0 | 200.0 | 4458.6 |
| | Mean | 82.5 | 74.7 | 200.0 | 4458.6 |
| | STDER | 0.3 | 2.7 | 0.0 | 17.1 |
| PBMC + LPS(5 ug/ml) + | 1st | 84.0 | 76.0 | 200.0 | 4547.7 |
| YDE043 0.005 nM | 2nd | 97.0 | 65.0 | 200.0 | 5320.3 |
| | 3rd | 119.0 | 61.0 | 200.0 | 6630.3 |
| | Mean | 100.0 | 67.3 | 200.0 | 5499.4 |
| | STDER | 10.2 | 4.5 | 0.0 | 607.8 |

TABLE 20
Bead name: TIMP-1

| | | | | | Concen- |
|---|---|---|---|---|---|
| | | | | Dilu- | tration |
| Sample | | MFI | Count | tion | (pg/ml) |
| Blank | 1st | 13.0 | 1.0 | 50.0 | 11.8 |
| | 2nd | 13.0 | 1.0 | 52.0 | 11.8 |
| | Mean | 13.0 | 1.0 | 51.0 | 11.8 |
| | STDER | 0.0 | 0.0 | 1.0 | 0.0 |
| PBMC + 0.5% DMSO | 1st | 26.5 | 200.0 | 50.0 | 6531.0 |
| | 2nd | 23.5 | 200.0 | 50.0 | 5800.6 |
| | 3rd | 22.0 | 200.0 | 52.0 | 5411.1 |
| | Mean | 24.0 | 200.0 | 50.7 | 5914.2 |
| | STDER | 1.3 | 0.0 | 0.7 | 328.2 |
| PBMC + LPS(5 ug/ml) | 1st | 26.5 | 200.0 | 50.0 | 6531.0 |
| | 2nd | 25.5 | 200.0 | 50.0 | 6293.9 |
| | 3rd | 25.5 | 200.0 | 50.0 | 6293.9 |
| | Mean | 25.8 | 200.0 | 50.0 | 6372.9 |
| | STDER | 0.3 | 0.0 | 0.0 | 79.0 |
| PBMC + YY101 500 nM | 1st | 29.0 | 200.0 | 50.0 | 7100.2 |
| | 2nd | 30.0 | 200.0 | 50.0 | 7319.6 |
| | Mean | 29.5 | 200.0 | 50.0 | 7209.9 |
| | STDER | 0.5 | 0.0 | 0.0 | 109.7 |
| PBMC + YY101 50 nM | 1st | 29.5 | 200.0 | 50.0 | 7210.4 |
| | 2nd | 27.0 | 200.0 | 68.0 | 6647.4 |
| | Mean | 28.3 | 200.0 | 59.0 | 6928.9 |
| | STDER | 1.3 | 0.0 | 9.0 | 281.5 |
| PBMC + YY101 5 nM | 1st | 26.5 | 200.0 | 50.0 | 6531.0 |
| | 2nd | 24.0 | 200.0 | 52.0 | 5926.6 |
| | Mean | 25.3 | 200.0 | 51.0 | 6228.8 |
| | STDER | 1.3 | 0.0 | 1.0 | 302.2 |
| PBMC + YY101 0.5 nM | 1st | 25.0 | 200.0 | 50.0 | 6173.1 |
| | 2nd | 28.5 | 200.0 | 64.0 | 6988.8 |
| | Mean | 26.8 | 200.0 | 57.0 | 6580.9 |
| | STDER | 1.8 | 0.0 | 7.0 | 407.8 |
| PBMC + YY101 0.05 uM | 1st | 46.0 | 200.0 | 54.0 | 10402.5 |
| | 2nd | 29.0 | 200.0 | 50.0 | 7100.2 |
| | Mean | 37.5 | 200.0 | 52.0 | 8751.3 |
| | STDER | 8.5 | 0.0 | 2.0 | 1651.2 |
| PBMC + LPS(5 ug/ml) + | 1st | 25.0 | 200.0 | 50.0 | 6173.1 |
| Xiidra 500 nM | 2nd | 23.0 | 200.0 | 57.0 | 5672.8 |
| | Mean | 24.0 | 200.0 | 53.5 | 5923.0 |
| | STDER | 1.0 | 0.0 | 3.5 | 250.1 |
| PBMC + LPS(5 ug/ml) + | 1st | 23.0 | 200.0 | 50.0 | 5672.8 |
| Xiidra 50 nM | 2nd | 22.5 | 200.0 | 50.0 | 5543.0 |
| | Mean | 22.8 | 200.0 | 50.0 | 5607.9 |
| | STDER | 0.3 | 0.0 | 0.0 | 64.9 |
| PBMC + LPS(5 ug/ml) + | 1st | 25.0 | 200.0 | 58.0 | 6173.1 |
| Xiidra 5 nM | 2nd | 26.0 | 200.0 | 52.0 | 6413.2 |
| | Mean | 25.5 | 200.0 | 55.0 | 6293.1 |
| | STDER | 0.5 | 0.0 | 3.0 | 120.0 |
| PBMC + LPS(5 ug/ml) + | 1st | 24.5 | 200.0 | 50.0 | 6050.7 |
| Xiidra 0.5 nM | 2nd | 25.0 | 200.0 | 50.0 | 6173.1 |
| | Mean | 24.8 | 200.0 | 50.0 | 6111.9 |
| | STDER | 0.3 | 0.0 | 0.0 | 61.2 |
| PBMC + LPS(5 ug/ml) + | 1st | 23.5 | 200.0 | 50.0 | 5800.6 |
| Xiidra 0.05 nM | 2nd | 24.0 | 200.0 | 55.0 | 5926.6 |
| | Mean | 23.8 | 200.0 | 52.5 | 5863.6 |
| | STDER | 0.3 | 0.0 | 2.5 | 63.0 |
| PBMC + LPS(5 ug/ml) + | 1st | 24.0 | 200.0 | 50.0 | 5926.6 |
| Xiidra 0.005 nM | 2nd | 22.5 | 200.0 | 50.0 | 5543.0 |
| | Mean | 23.3 | 200.0 | 50.0 | 5734.8 |
| | STDER | 0.8 | 0.0 | 0.0 | 191.8 |
| PBMC + LPS(5 ug/ml) + | 1st | 17.0 | 200.0 | 52.0 | 3935.4 |
| YY101 500 nM | 2nd | 17.0 | 200.0 | 67.0 | 3935.4 |
| | 3rd | 15.0 | 200.0 | 50.0 | 3220.1 |
| | Mean | 16.3 | 200.0 | 56.3 | 3696.9 |
| | STDER | 0.7 | 0.0 | 5.4 | 238.4 |
| PBMC + LPS(5 ug/ml) + | 1st | 17.0 | 200.0 | 50.0 | 3935.4 |
| YY101 50 nM | 2nd | 15.0 | 200.0 | 50.0 | 3220.1 |
| | 3rd | 17.0 | 200.0 | 54.0 | 3935.4 |
| | Mean | 16.3 | 200.0 | 51.3 | 3696.9 |
| | STDER | 0.7 | 0.0 | 1.3 | 238.4 |
| PBMC + LPS(5 ug/ml) + | 1st | 17.0 | 200.0 | 50.0 | 3935.4 |
| YY101 5 nM | 2nd | 17.0 | 200.0 | 57.0 | 3935.4 |
| | 3rd | 15.0 | 200.0 | 50.0 | 3220.1 |
| | Mean | 16.3 | 200.0 | 52.3 | 3696.9 |
| | STDER | 0.7 | 0.0 | 2.3 | 238.4 |
| PBMC + LPS(5 ug/ml) + | 1st | 17.0 | 200.0 | 50.0 | 3935.4 |
| YY101 0.5 nM | 2nd | 16.0 | 200.0 | 50.0 | 3590.9 |
| | 3rd | 20.0 | 200.0 | 50.0 | 4859.3 |
| | Mean | 17.7 | 200.0 | 50.0 | 4128.5 |
| | STDER | 1.2 | 0.0 | 0.0 | 378.7 |
| PBMC + LPS(5 ug/ml) + | 1st | 17.0 | 200.0 | 55.0 | 3935.4 |
| YY101 0.05 nM | 2nd | 16.0 | 200.0 | 50.0 | 3590.9 |
| | 3rd | 17.0 | 200.0 | 56.0 | 3935.4 |
| | Mean | 16.7 | 200.0 | 53.7 | 3820.5 |
| | STDER | 0.3 | 0.0 | 1.9 | 114.8 |
| PBMC + LPS(5 ug/ml) + | 1st | 18.0 | 200.0 | 50.0 | 4259.2 |
| YY101 0.005 nM | 2nd | 15.0 | 200.0 | 54.0 | 3220.1 |
| | 3rd | 18.0 | 200.0 | 57.0 | 4259.2 |
| | Mean | 17.0 | 200.0 | 53.7 | 3912.8 |
| | STDER | 1.0 | 0.0 | 2.0 | 346.4 |
| PBMC + LPS(5 ug/ml) + | 1st | 17.0 | 200.0 | 50.0 | 3935.4 |
| YDE011 500 nM | 2nd | 16.0 | 200.0 | 50.0 | 3590.9 |
| | 3rd | 17.0 | 200.0 | 52.0 | 3935.4 |
| | Mean | 16.7 | 200.0 | 50.7 | 3820.5 |
| | STDER | 0.3 | 0.0 | 0.7 | 114.8 |
| PBMC + LPS(5 ug/ml) + | 1st | 17.0 | 200.0 | 50.0 | 3935.4 |
| YDE011 50 nM | 2nd | 18.0 | 200.0 | 50.0 | 4259.2 |
| | 3rd | 19.0 | 200.0 | 55.0 | 4566.3 |
| | Mean | 18.0 | 200.0 | 51.7 | 4253.6 |
| | STDER | 0.6 | 0.0 | 1.7 | 182.1 |
| PBMC + LPS(5 ug/ml) + | 1st | 20.0 | 200.0 | 50.0 | 4859.3 |
| YDE011 5 nM | 2nd | 17.0 | 200.0 | 50.0 | 3935.4 |
| | 3rd | 16.0 | 200.0 | 50.0 | 3590.9 |
| | Mean | 17.7 | 200.0 | 50.0 | 4128.5 |
| | STDER | 1.2 | 0.0 | 0.0 | 378.7 |
| PBMC + LPS(5 ug/ml) + | 1st | 16.0 | 200.0 | 50.0 | 3590.9 |
| YDE011 0.5 nM | 2nd | 17.0 | 200.0 | 50.0 | 3935.4 |
| | 3rd | 19.5 | 200.0 | 54.0 | 4714.4 |
| | Mean | 17.5 | 200.0 | 51.3 | 4080.2 |
| | STDER | 1.0 | 0.0 | 1.3 | 332.3 |
| PBMC + LPS(5 ug/ml) + | 1st | 18.5 | 200.0 | 50.0 | 4414.6 |
| YDE011 0.05 nM | 2nd | 16.0 | 200.0 | 50.0 | 3590.9 |
| | 3rd | 20.0 | 200.0 | 50.0 | 4859.3 |
| | Mean | 18.2 | 200.0 | 50.0 | 4288.3 |
| | STDER | 1.2 | 0.0 | 0.0 | 371.6 |
| PBMC + LPS(5 ug/ml) + | 1st | 18.0 | 200.0 | 50.0 | 4259.2 |
| YDE011 0.005 nM | 2nd | 16.0 | 200.0 | 50.0 | 3590.9 |
| | 3rd | 18.0 | 200.0 | 50.0 | 4259.2 |
| | Mean | 17.3 | 200.0 | 50.0 | 4036.4 |
| | STDER | 0.7 | 0.0 | 0.0 | 222.8 |
| PBMC + LPS(5 ug/ml) + | 1st | 19.0 | 200.0 | 60.0 | 4566.3 |
| YDE043 500 nM | 2nd | 16.5 | 200.0 | 50.0 | 3766.0 |
| | 3rd | 19.0 | 200.0 | 50.0 | 4566.3 |
| | Mean | 18.2 | 200.0 | 53.3 | 4299.5 |
| | STDER | 0.8 | 0.0 | 3.3 | 266.7 |
| PBMC + LPS(5 ug/ml) + | 1st | 17.0 | 200.0 | 50.0 | 3935.4 |
| YDE043 50 nM | 2nd | 20.0 | 200.0 | 50.0 | 4859.3 |
| | 3rd | 18.0 | 200.0 | 58.0 | 4259.2 |
| | Mean | 18.3 | 200.0 | 52.7 | 4351.3 |
| | STDER | 0.9 | 0.0 | 2.7 | 270.6 |
| PBMC + LPS(5 ug/ml) + | 1st | 19.0 | 200.0 | 58.0 | 4566.3 |
| YDE043 5 nM | 2nd | 19.0 | 200.0 | 60.0 | 4566.3 |
| | 3rd | 18.0 | 200.0 | 54.0 | 4259.2 |
| | Mean | 18.7 | 200.0 | 57.3 | 4463.9 |
| | STDER | 0.3 | 0.0 | 1.8 | 102.3 |
| PBMC + LPS(5 ug/ml) + | 1st | 18.0 | 200.0 | 56.0 | 4259.2 |
| YDE043 0.5 nM | 2nd | 18.5 | 200.0 | 50.0 | 4414.6 |
| | 3rd | 17.0 | 200.0 | 51.0 | 3935.4 |
| | Mean | 17.8 | 200.0 | 52.3 | 4203.1 |
| | STDER | 0.4 | 0.0 | 1.9 | 141.2 |
| PBMC + LPS(5 ug/ml) + | 1st | 18.0 | 200.0 | 50.0 | 4259.2 |
| YDE043 0.05 nM | 2nd | 18.5 | 200.0 | 50.0 | 4414.6 |
| | 3rd | 16.0 | 200.0 | 50.0 | 3590.9 |
| | Mean | 17.5 | 200.0 | 50.0 | 4088.2 |
| | STDER | 0.8 | 0.0 | 0.0 | 252.7 |
| PBMC + LPS(5 ug/ml) + | 1st | 17.0 | 200.0 | 50.0 | 3935.4 |
| YDE043 0.005 nM | 2nd | 22.5 | 200.0 | 50.0 | 5543.0 |
| | 3rd | 19.0 | 200.0 | 54.0 | 4566.3 |
| | Mean | 19.5 | 200.0 | 51.3 | 4681.6 |
| | STDER | 1.6 | 0.0 | 1.3 | 467.7 |

TABLE 21
Bead name: Leptin

| | | | | | Concen- |
|---|---|---|---|---|---|
| | | | | Dilu- | tration |
| Sample | | MFI | Count | tion | (pg/ml) |
| Blank | 1st | 24.0 | 71.0 | 1.0 | 110.4 |
| | 2nd | 23.0 | 63.0 | 1.0 | 99.0 |
| | Mean | 23.5 | 67.0 | 1.0 | 104.7 |
| | STDER | 0.5 | 4.0 | 0.0 | 5.7 |
| PBMC + 0.5% DMSO | 1st | 23.0 | 63.0 | 200.0 | 19789.4 |
| | 2nd | 24.0 | 77.0 | 200.0 | 22079.2 |
| | 3rd | 22.0 | 73.0 | 200.0 | 17222.7 |
| | Mean | 23.0 | 71.0 | 200.0 | 19697.1 |
| | STDER | 0.6 | 4.2 | 0.0 | 1402.7 |
| PBMC + LPS(5 ug/ml) | 1st | 23.0 | 56.0 | 200.0 | 19789.4 |
| | 2nd | 22.0 | 51.0 | 200.0 | 17222.7 |
| | 3rd | 24.0 | 64.0 | 200.0 | 22079.2 |
| | Mean | 23.0 | 57.0 | 200.0 | 19697.1 |
| | STDER | 0.6 | 3.8 | 0.0 | 1402.7 |
| PBMC + YY101 500 nM | 1st | 23.0 | 88.0 | 200.0 | 19789.4 |
| | 2nd | 23.5 | 84.0 | 200.0 | 20963.1 |
| | Mean | 23.3 | 86.0 | 200.0 | 20376.3 |
| | STDER | 0.3 | 2.0 | 0.0 | 586.9 |
| PBMC + YY101 50 nM | 1st | 26.0 | 73.0 | 200.0 | 26106.3 |
| | 2nd | 24.0 | 63.0 | 200.0 | 22079.2 |
| | Mean | 25.0 | 68.0 | 200.0 | 24092.8 |
| | STDER | 1.0 | 5.0 | 0.0 | 2013.5 |
| PBMC + YY101 5 nM | 1st | 22.0 | 74.0 | 200.0 | 17222.7 |
| | 2nd | 23.5 | 58.0 | 200.0 | 20963.1 |
| | Mean | 22.8 | 66.0 | 200.0 | 19092.9 |
| | STDER | 0.8 | 8.0 | 0.0 | 1870.2 |
| PBMC + YY101 0.5 nM | 1st | 21.0 | 55.0 | 200.0 | 14234.9 |
| | 2nd | 22.0 | 62.0 | 200.0 | 17222.7 |
| | Mean | 21.5 | 58.5 | 200.0 | 15728.8 |
| | STDER | 0.5 | 3.5 | 0.0 | 1493.9 |
| PBMC + YY101 0.05 uM | 1st | 23.0 | 72.0 | 200.0 | 19789.4 |
| | 2nd | 22.0 | 61.0 | 200.0 | 17222.7 |
| | Mean | 22.5 | 66.5 | 200.0 | 18506.0 |
| | STDER | 0.5 | 5.5 | 0.0 | 1283.3 |
| PBMC + LPS(5 ug/ml) + | 1st | 26.0 | 50.0 | 200.0 | 26106.3 |
| Xiidra 500 nM | 2nd | 22.0 | 60.0 | 200.0 | 17222.7 |
| | Mean | 24.0 | 55.0 | 200.0 | 21664.5 |
| | STDER | 2.0 | 5.0 | 0.0 | 4441.8 |
| PBMC + LPS(5 ug/ml) + | 1st | 25.0 | 53.0 | 200.0 | 24169.3 |
| Xiidra 50 nM | 2nd | 22.0 | 92.0 | 200.0 | 17222.7 |
| | Mean | 23.5 | 72.5 | 200.0 | 20696.0 |
| | STDER | 1.5 | 19.5 | 0.0 | 3473.3 |
| PBMC + LPS(5 ug/ml) + | 1st | 22.0 | 77.0 | 200.0 | 17222.7 |
| Xiidra 5 nM | 2nd | 22.0 | 50.0 | 200.0 | 17222.7 |
| | Mean | 22.0 | 63.5 | 200.0 | 17222.7 |
| | STDER | 0.0 | 13.5 | 0.0 | 0.0 |
| PBMC + LPS(5 ug/ml) + | 1st | 20.0 | 72.0 | 200.0 | 10481.4 |
| Xiidra 0.5 nM | 2nd | 20.0 | 66.0 | 200.0 | 10481.4 |
| | Mean | 20.0 | 69.0 | 200.0 | 10481.4 |
| | STDER | 0.0 | 3.0 | 0.0 | 0.0 |
| PBMC + LPS(5 ug/ml) + | 1st | 22.0 | 81.0 | 200.0 | 17222.7 |
| Xiidra 0.05 nM | 2nd | 22.5 | 58.0 | 200.0 | 18547.4 |
| | Mean | 22.3 | 69.5 | 200.0 | 17885.0 |
| | STDER | 0.3 | 11.5 | 0.0 | 662.3 |
| PBMC + LPS(5 ug/ml) + | 1st | 21.0 | 66.0 | 200.0 | 14234.9 |
| Xiidra 0.005 nM | 2nd | 22.0 | 55.0 | 200.0 | 17222.7 |
| | Mean | 21.5 | 60.5 | 200.0 | 15728.8 |
| | STDER | 0.5 | 5.5 | 0.0 | 1493.9 |
| PBMC + LPS(5 ug/ml) + | 1st | 23.0 | 53.0 | 200.0 | 19789.4 |
| YY101 500 nM | 2nd | 22.0 | 72.0 | 200.0 | 17222.7 |
| | 3rd | 23.0 | 66.0 | 200.0 | 19789.4 |
| | Mean | 22.7 | 63.7 | 200.0 | 18933.8 |
| | STDER | 0.3 | 5.6 | 0.0 | 855.5 |
| PBMC + LPS(5 ug/ml) + | 1st | 22.0 | 59.0 | 200.0 | 17222.7 |
| YY101 50 nM | 2nd | 21.0 | 65.0 | 200.0 | 14234.9 |
| | 3rd | 21.0 | 71.0 | 200.0 | 14234.9 |
| | Mean | 21.3 | 65.0 | 200.0 | 15230.8 |
| | STDER | 0.3 | 3.5 | 0.0 | 995.9 |
| PBMC + LPS(5 ug/ml) + | 1st | 24.0 | 60.0 | 200.0 | 22079.2 |
| YY101 5 nM | 2nd | 22.0 | 74.0 | 200.0 | 17222.7 |
| | 3rd | 25.0 | 63.0 | 200.0 | 24169.3 |
| | Mean | 23.7 | 65.7 | 200.0 | 21157.1 |
| | STDER | 0.9 | 4.3 | 0.0 | 2057.6 |
| PBMC + LPS(5 ug/ml) + | 1st | 22.0 | 52.0 | 200.0 | 17222.7 |
| YY101 0.5 nM | 2nd | 22.0 | 71.0 | 200.0 | 17222.7 |
| | 3rd | 21.0 | 81.0 | 200.0 | 14234.9 |
| | Mean | 21.7 | 68.0 | 200.0 | 16226.8 |
| | STDER | 0.3 | 8.5 | 0.0 | 995.9 |
| PBMC + LPS(5 ug/ml) + | 1st | 22.0 | 56.0 | 200.0 | 17222.7 |
| YY101 0.05 nM | 2nd | 21.5 | 70.0 | 200.0 | 15795.3 |
| | 3rd | 22.0 | 52.0 | 200.0 | 17222.7 |
| | Mean | 21.8 | 59.3 | 200.0 | 16746.9 |
| | STDER | 0.2 | 5.5 | 0.0 | 475.8 |
| PBMC + LPS(5 ug/ml) + | 1st | 23.0 | 62.0 | 200.0 | 19789.4 |
| YY101 0.005 nM | 2nd | 20.0 | 54.0 | 200.0 | 10481.4 |
| | 3rd | 23.0 | 59.0 | 200.0 | 19789.4 |
| | Mean | 22.0 | 58.3 | 200.0 | 16686.7 |
| | STDER | 1.0 | 2.3 | 0.0 | 3102.7 |
| PBMC + LPS(5 ug/ml) + | 1st | 23.0 | 63.0 | 200.0 | 19789.4 |
| YDE011 500 nM | 2nd | 13.0 | 79.0 | 200.0 | <98.95 |
| | 3rd | 19.5 | 62.0 | 200.0 | 8003.2 |
| | Mean | 18.5 | 68.0 | 200.0 | 13896.3 |
| | STDER | 2.9 | 5.5 | 0.0 | 5893.1 |
| PBMC + LPS(5 ug/ml) + | 1st | 20.0 | 66.0 | 200.0 | 10481.4 |
| YDE011 50 nM | 2nd | 21.0 | 65.0 | 200.0 | 14234.9 |
| | 3rd | 12.0 | 60.0 | 200.0 | <98.95 |
| | Mean | 17.7 | 63.7 | 200.0 | 12358.1 |
| | STDER | 2.8 | 1.9 | 0.0 | 1876.7 |
| PBMC + LPS(5 ug/ml) + | 1st | 17.0 | 52.0 | 200.0 | <98.95 |
| YDE011 5 nM | 2nd | 19.0 | 60.0 | 200.0 | 4332.6 |
| | 3rd | 22.0 | 62.0 | 200.0 | 17222.7 |
| | Mean | 19.3 | 58.0 | 200.0 | 10777.7 |
| | STDER | 1.5 | 3.1 | 0.0 | 6445.0 |
| PBMC + LPS(5 ug/ml) + | 1st | 20.0 | 76.0 | 200.0 | 10481.4 |
| YDE011 0.5 nM | 2nd | 19.0 | 91.0 | 200.0 | 4332.6 |
| | 3rd | 21.0 | 67.0 | 200.0 | 14234.9 |
| | Mean | 20.0 | 78.0 | 200.0 | 9683.0 |
| | STDER | 0.6 | 7.0 | 0.0 | 2886.3 |
| PBMC + LPS(5 ug/ml) + | 1st | 23.0 | 74.0 | 200.0 | 19789.4 |
| YDE011 0.05 nM | 2nd | 20.0 | 80.0 | 200.0 | 10481.4 |
| | 3rd | 21.0 | 81.0 | 200.0 | 14234.9 |
| | Mean | 21.3 | 78.3 | 200.0 | 14835.2 |
| | STDER | 0.9 | 2.2 | 0.0 | 2703.7 |
| PBMC + LPS(5 ug/ml) + | 1st | 22.0 | 50.0 | 200.0 | 17222.7 |
| YDE011 0.005 nM | 2nd | 22.0 | 59.0 | 200.0 | 17222.7 |
| | 3rd | 20.0 | 75.0 | 200.0 | 10481.4 |
| | Mean | 21.3 | 61.3 | 200.0 | 14975.6 |
| | STDER | 0.7 | 7.3 | 0.0 | 2247.1 |
| PBMC + LPS(5 ug/ml) + | 1st | 22.0 | 63.0 | 200.0 | 17222.7 |
| YDE043 500 nM | 2nd | 19.0 | 65.0 | 200.0 | 4332.6 |
| | 3rd | 20.0 | 50.0 | 200.0 | 10481.4 |
| | Mean | 20.3 | 59.3 | 200.0 | 10678.9 |
| | STDER | 0.9 | 4.7 | 0.0 | 3722.4 |
| PBMC + LPS(5 ug/ml) + | 1st | 21.5 | 54.0 | 200.0 | 15795.3 |
| YDE043 50 nM | 2nd | 23.0 | 65.0 | 200.0 | 19789.4 |
| | 3rd | 23.0 | 70.0 | 200.0 | 19789.4 |
| | Mean | 22.5 | 63.0 | 200.0 | 18458.0 |
| | STDER | 0.5 | 4.7 | 0.0 | 1331.4 |
| PBMC + LPS(5 ug/ml) + | 1st | 23.0 | 55.0 | 200.0 | 19789.4 |
| YDE043 5 nM | 2nd | 20.5 | 60.0 | 200.0 | 12493.0 |
| | 3rd | 21.0 | 79.0 | 200.0 | 14234.9 |
| | Mean | 21.5 | 64.7 | 200.0 | 15505.7 |
| | STDER | 0.8 | 7.3 | 0.0 | 2200.0 |
| PBMC + LPS(5 ug/ml) + | 1st | 20.5 | 54.0 | 200.0 | 12493.0 |
| YDE043 0.5 nM | 2nd | 22.0 | 64.0 | 200.0 | 17222.7 |
| | 3rd | 22.0 | 63.0 | 200.0 | 17222.7 |
| | Mean | 21.5 | 60.3 | 200.0 | 15646.1 |
| | STDER | 0.5 | 3.2 | 0.0 | 1576.6 |
| PBMC + LPS(5 ug/ml) + | 1st | 20.0 | 63.0 | 200.0 | 10481.4 |
| YDE043 0.05 nM | 2nd | 18.5 | 60.0 | 200.0 | <98.95 |
| | 3rd | 18.0 | 62.0 | 200.0 | <98.95 |
| | Mean | 18.8 | 61.7 | 200.0 | 10481.4 |
| | STDER | 0.6 | 0.9 | 0.0 | #DIV/0! |
| PBMC + LPS(5 ug/ml) + | 1st | 22.5 | 58.0 | 200.0 | 18547.4 |
| YDE043 0.005 nM | 2nd | 24.0 | 60.0 | 200.0 | 22079.2 |
| | 3rd | 22.5 | 56.0 | 200.0 | 18547.4 |
| | Mean | 23.0 | 58.0 | 200.0 | 19724.6 |
| | STDER | 0.5 | 1.2 | 0.0 | 1177.3 |

TABLE 22
Bead name: MMP-9

| | | | | | Concen- |
|---|---|---|---|---|---|
| | | | | Dilu- | tration |
| Sample | | MFI | Count | tion | (pg/ml) |
| Blank | 1st | 29.5 | 50.0 | 1.0 | 20.7 |
| | 2nd | 34.0 | 64.0 | 1.0 | 27.8 |
| | Mean | 31.8 | 57.0 | 1.0 | 24.3 |
| | STDER | 2.3 | 7.0 | 0.0 | 3.5 |
| PBMC + 0.5% DMSO | 1st | 56.0 | 66.0 | 200.0 | 12836.1 |
| | 2nd | 39.0 | 76.0 | 200.0 | 7158.5 |
| | 3rd | 37.0 | 93.0 | 200.0 | 6512.8 |
| | Mean | 44.0 | 78.3 | 200.0 | 8835.8 |
| | STDER | 6.0 | 7.9 | 0.0 | 2008.8 |
| PBMC + LPS(5 ug/ml) | 1st | 33.0 | 87.0 | 200.0 | 5239.4 |
| | 2nd | 35.0 | 77.0 | 200.0 | 5872.9 |
| | 3rd | 33.0 | 83.0 | 200.0 | 5239.4 |
| | Mean | 33.7 | 82.3 | 200.0 | 5450.5 |
| | STDER | 0.7 | 2.9 | 0.0 | 211.2 |
| PBMC + YY101 500 nM | 1st | 48.0 | 69.0 | 200.0 | 10126.8 |
| | 2nd | 54.0 | 92.0 | 200.0 | 12153.3 |
| | Mean | 51.0 | 80.5 | 200.0 | 11140.0 |
| | STDER | 3.0 | 11.5 | 0.0 | 1013.3 |
| PBMC + YY101 50 nM | 1st | 53.0 | 89.0 | 200.0 | 11813.2 |
| | 2nd | 46.0 | 70.0 | 200.0 | 9459.2 |
| | Mean | 49.5 | 79.5 | 200.0 | 10636.2 |
| | STDER | 3.5 | 9.5 | 0.0 | 1177.0 |
| PBMC + YY101 5 nM | 1st | 67.5 | 78.0 | 200.0 | 16823.6 |
| | 2nd | 52.0 | 81.0 | 200.0 | 11474.0 |
| | Mean | 59.8 | 79.5 | 200.0 | 14148.8 |
| | STDER | 7.8 | 1.5 | 0.0 | 2674.8 |
| PBMC + YY101 0.5 nM | 1st | 56.0 | 82.0 | 200.0 | 12836.1 |
| | 2nd | 77.0 | 73.0 | 200.0 | 20186.4 |
| | Mean | 66.5 | 77.5 | 200.0 | 16511.2 |
| | STDER | 10.5 | 4.5 | 0.0 | 3675.1 |
| PBMC + YY101 0.05 uM | 1st | 79.0 | 69.0 | 200.0 | 20901.3 |
| | 2nd | 57.0 | 74.0 | 200.0 | 13178.7 |
| | Mean | 68.0 | 71.5 | 200.0 | 17040.0 |
| | STDER | 11.0 | 2.5 | 0.0 | 3861.3 |
| PBMC + LPS(5 ug/ml) + | 1st | 35.0 | 73.0 | 200.0 | 5872.9 |
| Xiidra 500 nM | 2nd | 35.0 | 65.0 | 200.0 | 5872.9 |
| | Mean | 35.0 | 69.0 | 200.0 | 5872.9 |
| | STDER | 0.0 | 4.0 | 0.0 | 0.0 |
| PBMC + LPS(5 ug/ml) + | 1st | 35.0 | 63.0 | 200.0 | 5872.9 |
| Xiidra 50 nM | 2nd | 37.0 | 79.0 | 200.0 | 6512.8 |
| | Mean | 36.0 | 71.0 | 200.0 | 6192.8 |
| | STDER | 1.0 | 8.0 | 0.0 | 319.9 |
| PBMC + LPS(5 ug/ml) + | 1st | 32.0 | 76.0 | 200.0 | 4925.2 |
| Xiidra 5 nM | 2nd | 37.0 | 85.0 | 200.0 | 6512.8 |
| | Mean | 34.5 | 80.5 | 200.0 | 5719.0 |
| | STDER | 2.5 | 4.5 | 0.0 | 793.8 |
| PBMC + LPS(5 ug/ml) + | 1st | 27.5 | 72.0 | 200.0 | 3535.8 |
| Xiidra 0.5 nM | 2nd | 31.0 | 83.0 | 200.0 | 4612.9 |
| | Mean | 29.3 | 77.5 | 200.0 | 4074.3 |
| | STDER | 1.8 | 5.5 | 0.0 | 538.5 |
| PBMC + LPS(5 ug/ml) + | 1st | 30.0 | 102.0 | 200.0 | 4302.5 |
| Xiidra 0.05 nM | 2nd | 31.5 | 78.0 | 200.0 | 4768.8 |
| | Mean | 30.8 | 90.0 | 200.0 | 4535.6 |
| | STDER | 0.8 | 12.0 | 0.0 | 233.2 |
| PBMC + LPS(5 ug/ml) + | 1st | 30.0 | 77.0 | 200.0 | 4302.5 |
| Xiidra 0.005 nM | 2nd | 30.0 | 76.0 | 200.0 | 4302.5 |
| | Mean | 30.0 | 76.5 | 200.0 | 4302.5 |
| | STDER | 0.0 | 0.5 | 0.0 | 0.0 |
| PBMC + LPS(5 ug/ml) + | 1st | 31.0 | 64.0 | 200.0 | 4612.9 |
| YY101 500 nM | 2nd | 33.0 | 71.0 | 200.0 | 5239.4 |
| | 3rd | 34.0 | 64.0 | 200.0 | 5555.3 |
| | Mean | 32.7 | 66.3 | 200.0 | 5135.8 |
| | STDER | 0.9 | 2.3 | 0.0 | 276.9 |
| PBMC + LPS(5 ug/ml) + | 1st | 33.0 | 80.0 | 200.0 | 5239.4 |
| YY101 50 nM | 2nd | 32.0 | 76.0 | 200.0 | 4925.2 |
| | 3rd | 34.0 | 76.0 | 200.0 | 5555.3 |
| | Mean | 33.0 | 77.3 | 200.0 | 5239.9 |
| | STDER | 0.6 | 1.3 | 0.0 | 181.9 |
| PBMC + LPS(5 ug/ml) + | 1st | 33.0 | 76.0 | 200.0 | 5239.4 |
| YY101 5 nM | 2nd | 30.0 | 74.0 | 200.0 | 4302.5 |
| | 3rd | 35.0 | 79.0 | 200.0 | 5872.9 |
| | Mean | 32.7 | 76.3 | 200.0 | 5138.2 |
| | STDER | 1.5 | 1.5 | 0.0 | 456.1 |
| PBMC + LPS(5 ug/ml) + | 1st | 33.0 | 73.0 | 200.0 | 5239.4 |
| YY101 0.5 nM | 2nd | 33.0 | 74.0 | 200.0 | 5239.4 |
| | 3rd | 33.0 | 60.0 | 200.0 | 5239.4 |
| | Mean | 33.0 | 69.0 | 200.0 | 5239.4 |
| | STDER | 0.0 | 4.5 | 0.0 | 0.0 |
| PBMC + LPS(5 ug/ml) + | 1st | 33.0 | 69.0 | 200.0 | 5239.4 |
| YY101 0.05 nM | 2nd | 32.0 | 77.0 | 200.0 | 4925.2 |
| | 3rd | 31.5 | 68.0 | 200.0 | 4768.8 |
| | Mean | 32.2 | 71.3 | 200.0 | 4977.8 |
| | STDER | 0.4 | 2.8 | 0.0 | 138.4 |
| PBMC + LPS(5 ug/ml) + | 1st | 28.0 | 76.0 | 200.0 | 3688.0 |
| YY101 0.005 nM | 2nd | 32.0 | 69.0 | 200.0 | 4925.2 |
| | 3rd | 33.0 | 57.0 | 200.0 | 5239.4 |
| | Mean | 31.0 | 67.3 | 200.0 | 4617.5 |
| | STDER | 1.5 | 5.5 | 0.0 | 473.5 |
| PBMC + LPS(5 ug/ml) + | 1st | 31.0 | 75.0 | 200.0 | 4612.9 |
| YDE011 500 nM | 2nd | 18.5 | 76.0 | 200.0 | 931.9 |
| | 3rd | 25.0 | 77.0 | 200.0 | 2784.3 |
| | Mean | 24.8 | 76.0 | 200.0 | 2776.4 |
| | STDER | 3.6 | 0.6 | 0.0 | 1062.6 |
| PBMC + LPS(5 ug/ml) + | 1st | 24.5 | 74.0 | 200.0 | 2636.1 |
| YDE011 50 nM | 2nd | 28.0 | 71.0 | 200.0 | 3688.0 |
| | 3rd | 20.0 | 62.0 | 200.0 | 1342.1 |
| | Mean | 24.2 | 69.0 | 200.0 | 2555.4 |
| | STDER | 2.3 | 3.6 | 0.0 | 678.4 |
| PBMC + LPS(5 ug/ml) + | 1st | 22.0 | 77.0 | 200.0 | 1907.3 |
| YDE011 5 nM | 2nd | 28.0 | 65.0 | 200.0 | 3688.0 |
| | 3rd | 30.0 | 77.0 | 200.0 | 4302.5 |
| | Mean | 26.7 | 73.0 | 200.0 | 3299.3 |
| | STDER | 2.4 | 4.0 | 0.0 | 718.2 |
| PBMC + LPS(5 ug/ml) + | 1st | 27.0 | 92.0 | 200.0 | 3384.2 |
| YDE011 0.5 nM | 2nd | 28.0 | 80.0 | 200.0 | 3688.0 |
| | 3rd | 26.5 | 66.0 | 200.0 | 3233.2 |
| | Mean | 27.2 | 79.3 | 200.0 | 3435.2 |
| | STDER | 0.4 | 7.5 | 0.0 | 133.7 |
| PBMC + LPS(5 ug/ml) + | 1st | 31.0 | 65.0 | 200.0 | 4612.9 |
| YDE011 0.05 nM | 2nd | 31.0 | 79.0 | 200.0 | 4612.9 |
| | 3rd | 30.0 | 80.0 | 200.0 | 4302.5 |
| | Mean | 30.7 | 74.7 | 200.0 | 4509.4 |
| | STDER | 0.3 | 4.8 | 0.0 | 103.5 |
| PBMC + LPS(5 ug/ml) + | 1st | 29.5 | 60.0 | 200.0 | 4148.1 |
| YDE011 0.005 nM | 2nd | 31.0 | 60.0 | 200.0 | 4612.9 |
| | 3rd | 29.0 | 82.0 | 200.0 | 3994.2 |
| | Mean | 29.8 | 67.3 | 200.0 | 4251.7 |
| | STDER | 0.6 | 7.3 | 0.0 | 186.0 |
| PBMC + LPS(5 ug/ml) + | 1st | 28.0 | 72.0 | 200.0 | 3688.0 |
| YDE043 500 nM | 2nd | 32.0 | 70.0 | 200.0 | 4925.2 |
| | 3rd | 28.0 | 85.0 | 200.0 | 3688.0 |
| | Mean | 29.3 | 75.7 | 200.0 | 4100.4 |
| | STDER | 1.3 | 4.7 | 0.0 | 412.4 |
| PBMC + LPS(5 ug/ml) + | 1st | 28.0 | 75.0 | 200.0 | 3688.0 |
| YDE043 50 nM | 2nd | 28.0 | 70.0 | 200.0 | 3688.0 |
| | 3rd | 30.0 | 81.0 | 200.0 | 4302.5 |
| | Mean | 28.7 | 75.3 | 200.0 | 3892.8 |
| | STDER | 0.7 | 3.2 | 0.0 | 204.8 |
| PBMC + LPS(5 ug/ml) + | 1st | 30.0 | 50.0 | 200.0 | 4302.5 |
| YDE043 5 nM | 2nd | 28.0 | 67.0 | 200.0 | 3688.0 |
| | 3rd | 27.5 | 72.0 | 200.0 | 3535.8 |
| | Mean | 28.5 | 63.0 | 200.0 | 3842.1 |
| | STDER | 0.8 | 6.7 | 0.0 | 234.3 |
| PBMC + LPS(5 ug/ml) + | 1st | 28.0 | 60.0 | 200.0 | 3688.0 |
| YDE043 0.5 nM | 2nd | 28.0 | 86.0 | 200.0 | 3688.0 |
| | 3rd | 26.5 | 64.0 | 200.0 | 3233.2 |
| | Mean | 27.5 | 70.0 | 200.0 | 3536.4 |
| | STDER | 0.5 | 8.1 | 0.0 | 151.6 |
| PBMC + LPS(5 ug/ml) + | 1st | 29.0 | 75.0 | 200.0 | 3994.2 |
| YDE043 0.05 nM | 2nd | 29.5 | 70.0 | 200.0 | 4148.1 |
| | 3rd | 28.0 | 76.0 | 200.0 | 3688.0 |
| | Mean | 28.8 | 73.7 | 200.0 | 3943.4 |
| | STDER | 0.4 | 1.9 | 0.0 | 135.2 |
| PBMC + LPS(5 ug/ml) + | 1st | 29.0 | 53.0 | 200.0 | 3994.2 |
| YDE043 0.005 nM | 2nd | 34.0 | 69.0 | 200.0 | 5555.3 |
| | 3rd | 36.0 | 79.0 | 200.0 | 6192.0 |
| | Mean | 33.0 | 67.0 | 200.0 | 5247.2 |
| | STDER | 2.1 | 7.6 | 0.0 | 652.9 |

### Example 9: Investigate the Effects of YY-101, YDE-011 & YDE-043 Compounds on Various Cytokine and Chemokine Release in Human Peripheral Blood Mononuclear Cells (PBMCs) (Study 2)

### Background and Purpose

The objective of this study was to investigate the effects of YY-101, YDE-011 and YDE-043 compound on various cytokine and chemokine release in human peripheral blood mononuclear cells (hPBMCs) stimulated by known stimulants LPS.

To achieve this goal, this study was conducted to establish the efficacious dose-response curve of YY-101, YDE-011 and YDE-043 up to 30 cytokine and chemokine release in hPBMC induced by LPS.

### Materials & Methods

Human PBMCs frozen purchased from ATCC in a cryo-preservative were thawed, washed with Hank's Balanced Salt Solution containing 10% fetal bovine serum, and seeded onto a 24-well plate at a density of 0.5×106 cells/well with culture RPMI media containing 10% fetal bovine serum in growth media in a 24 well plate for 24 hrs and maintained at 37° C. in an atmosphere of 95% air and 5% CO2. At the end of 24 hr culture, cells are pretreated with YY-101, YDE-011 and YDE-043 or reference compound, Xiidra® at various concentrations for 2 h before stimulation. An equal volume of 0.5% DMSO is used as a vehicle control. Then, the cells are treated with LPS. Supernatants from treated cells are harvested at 24 hr post-LPS treatment and placed in 96 well for Luminex cytokine & chemokine profiling using a Luminex 200 multiplex assay.

### Reagents and Solutions

Human PBMCs (ATCC, USA)
Lipopolysaccharides from Escherichia coli (Sigma Aldrich, USA)
Human magnetic Luminex assay kit (R&D System, USA)
Xiidra® (Shire, USA)
Test materials (YY-101, YDE-011 and YDE-043) were stored in a deep freezer (−20° C.).

### Cell Culture

Human PBMCs frozen in a cryopreserve was thawed, washed with Hank's Balanced Salt Solution containing 10% Fetal bovine serum, and seeded onto a 24-well plate at a density of 0.5×10⁶ cells/well with culture RPMI media containing 10% Fetal bovine serum in growth media in a 24 well plate for 24 hrs and maintained at 37° C. in an atmosphere of 95% air and 5% CO2.

Treatment Human PBMCs were pretreated with YY-101, YDE-011 and YDE-043 or reference compound; Xiidra® at various concentration (ranging from 1 nM to 100 uM) for 2 h before stimulation. An equal volume of 0.5% DMSO was used as a vehicle control. Then, the cells were treated with LPS at 5 ug/ml. The supernatants from treated cells were harvested at 24 h and placed in a 96 well for Luminex cytokine & chemokine profiling using a Luminex 200 multiplex assay (Luminex; R&D system, USA).

The main study arm with triplicate wells per group in a 24-well plate:
1. Blank
2. PBMC: 0.5% DMSO
3. PBMC+LPS (5 ug/ml)
4. PBMC+YY-101 100 uM
5. PBMC+YY-101 10 uM
6. PBMC+YY-101 1 uM
7. PBMC+YY-101 0.1 uM
8. PBMC+YY-101 0.01 uM
9. PBMC+LPS (5 ug/ml)+Xiidra®100 uM
10. PBMC+LPS (5 ug/ml)+Xiidra® 10 uM
11. PBMC+LPS (5 ug/ml)+Xiidra® 1 uM
12. PBMC+LPS (5 ug/ml)+Xiidra®0.1 uM
13. PBMC+LPS (5 ug/ml)+Xiidra®0.01 uM
14. PBMC+LPS (5 ug/ml)+Xiidra®0.001 uM
15. PBMC+LPS (5 ug/ml)+YY-101 100 uM
16. PBMC+LPS (5 ug/ml)+YY-101 10 uM
17. PBMC+LPS (5 ug/ml)+YY-101 1 uM
18. PBMC+LPS (5 ug/ml)+YY-101 0.1 uM
19. PBMC+LPS (5 ug/ml)+YY-101 0.01 uM
20. PBMC+LPS (5 ug/ml)+YY-101 0.001 uM
21. PBMC+LPS (5 ug/ml)+YDE-011 100 uM
22. PBMC+LPS (5 ug/ml)+YDE-011 10 uM
23. PBMC+LPS (5 ug/ml)+YDE-011 1 uM
24. PBMC+LPS (5 ug/ml)+YDE-011 0.1 uM
25. PBMC+LPS (5 ug/ml)+YDE-011 0.01 uM
26. PBMC+LPS (5 ug/ml)+YDE-011 0.001 uM
27. PBMC+LPS (5 ug/ml)+YDE-043 100 uM
28. PBMC+LPS (5 ug/ml)+YDE-043 10 uM
29. PBMC+LPS (5 ug/ml)+YDE-043 1 uM
30. PBMC+LPS (5 ug/ml)+YDE-043 0.1 uM
31. PBMC+LPS (5 ug/ml)+YDE-043 0.01 uM
32. PBMC+LPS (5 ug/ml)+YDE-043 0.001 uM

### Cell Count

The number of PBMCs was measured after 24 hr of treatment. The culture both and trypan blue were diluted 1:1 and 10 ul was added to Countess cell counting chamber (Thermo fisher, USA). The number of PBMCs was measured using a Countess automated cell counter (Thermo fisher, USA).

### Multiplex Assay

Multiplex assay was used to measure 30 cytokines and chemokines. The 1:2, 1:10 and 1:200 diluted cell supernatant and serial dilution of the standard were dispensed into a 96 well plate at 50 ul/well. Then, the pre-mixed cocktail of antibody-coated magnetic beads was dispensed at 50 ul/well and incubated at room temperature for 2 h in a horizontal orbital microplate shaker at 800±500 rpm. The beads were washed using a magnetic device to prevent loss. The biotin-antibody was dispensed in 50 ul of each well and incubated at room temperature for 1 h in a shaker at 800±500 rpm. After washing, streptavidin-PE was added at 50 ul/well and incubated at room temperature for 30 min in a shaker under the same conditions. Finally, after washing, wash buffer (100 ul/well) was added to the plate and incubated in a shaker for 2 min. The Luminex™ 200 setting were set according to the manufacturer's protocol. The data was calculated with a standard five-parameter logistic nonlinear regression analysis of the data (xPonent software 4.2, USA). The levels of cytokine/chemokine are normalized to the live cell counts (1×10⁵ cells/mL) and presented as concentrations per mL (pg/mL or ng/mL).

Below is the list of cytokines and chemokines either in 21-plex, 5-plex and two 2-plex (Table 23).

TABLE 23

| 21 plex | 1:2 dilution | 5 plex | 1:10 dilution |
|---|---|---|---|
| Human | Cytokine/Chemokine | Human | Cytokine/Chemokine |
| 1 | MCP-1 (CCL2) | 1 | MIP-1 alpha (CCL3) |
| 2 | RANTES (CCL5) | 2 | MIP-1 beta (CCL4) |
| 3 | MIP-3alpha (CCL20) | 3 | IL-1 beta |
| 4 | Fractalkine (CX3CL1) | 4 | IL-10 |
| 5 | Fas-L | 5 | TNF-alpha |
| 6 | GM-CSF | | |
| 7 | ICAM-1 | | |
| 8 | IFN-gamma | | |
| 9 | IL-1 alpha | | |
| 10 | IL-2 | | |
| 11 | IL-4 | | |
| 12 | IL-5 | | |
| 13 | IL-13 | | |
| 14 | IL-17A | | |
| 15 | Leptin | | |
| 16 | MMP-3 | | |
| 17 | MMP-8 | | |
| 18 | MMP-9 | | |
| 19 | RAGE | | |
| 20 | L-Selectin | | |
| 21 | VEGF-D | | |
| 2-plex | 1:2 dilution | 2-plex | 1:200 dilution |
| Human | Cytokine/Chemokine list | Human | Cytokine/Chemokine |
| 1 | TIMP-1 | 1 | IL-6 |
| 2 | VEGF-A | 2 | IL-8 (CXCL8) |

### Compound Delivery & Formulation

The concentration of compound was prepared according to the information provided by the client.

All compound was serially diluted in from the stock concentrations and the final concentration of DMSO was not exceed 0.5% DMSO. All compounds were prepared on the same day of the treatment.

### Results

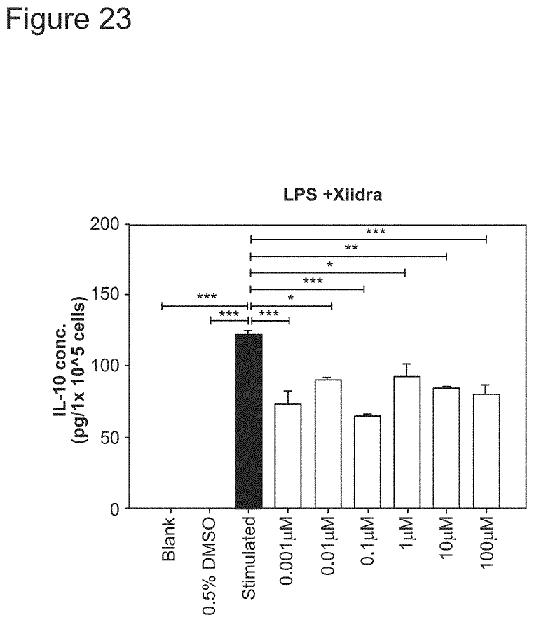

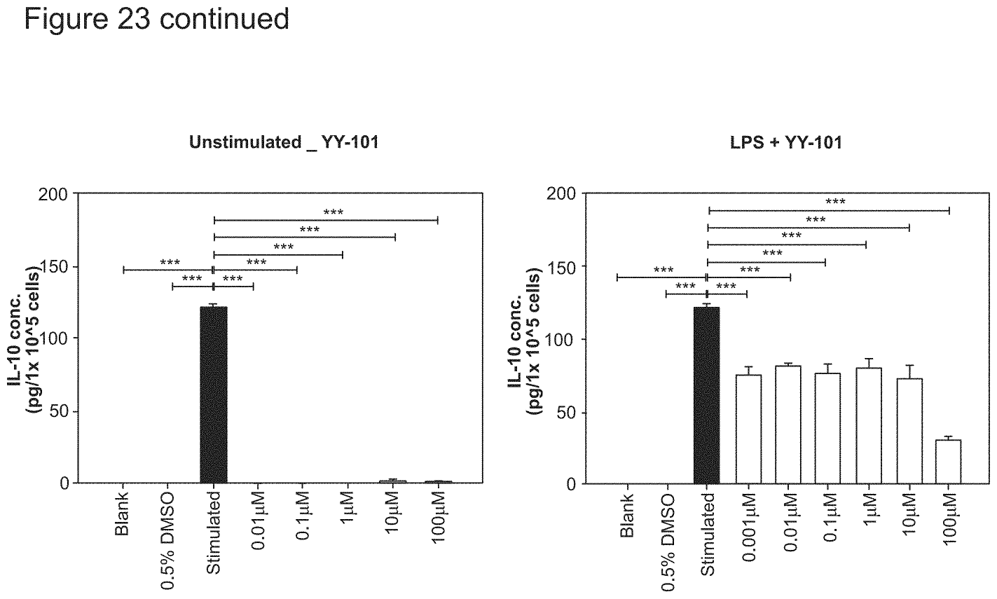

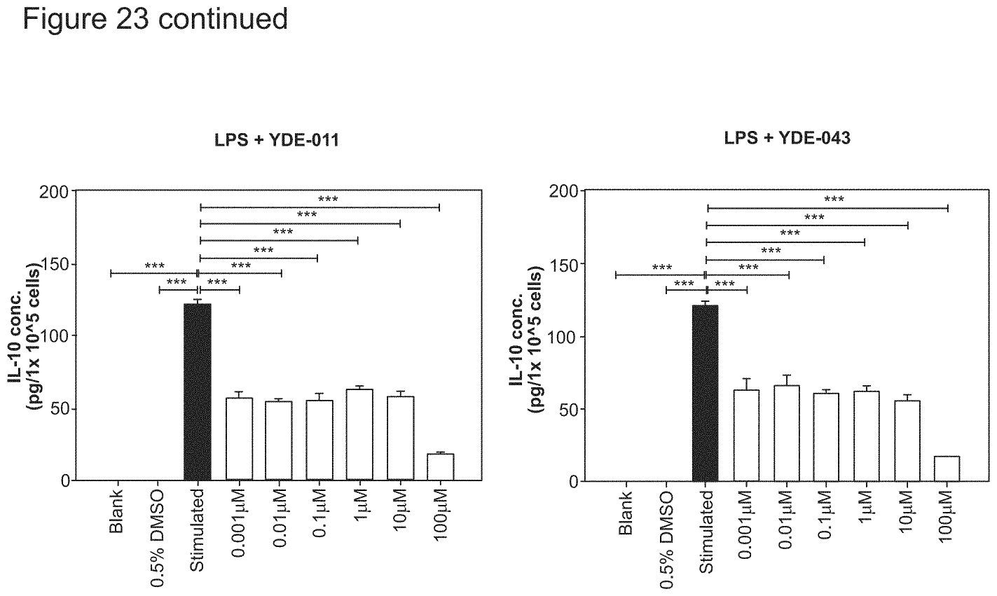

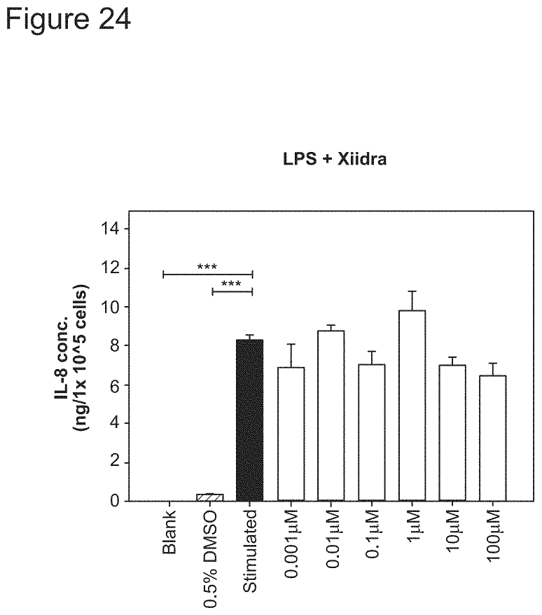

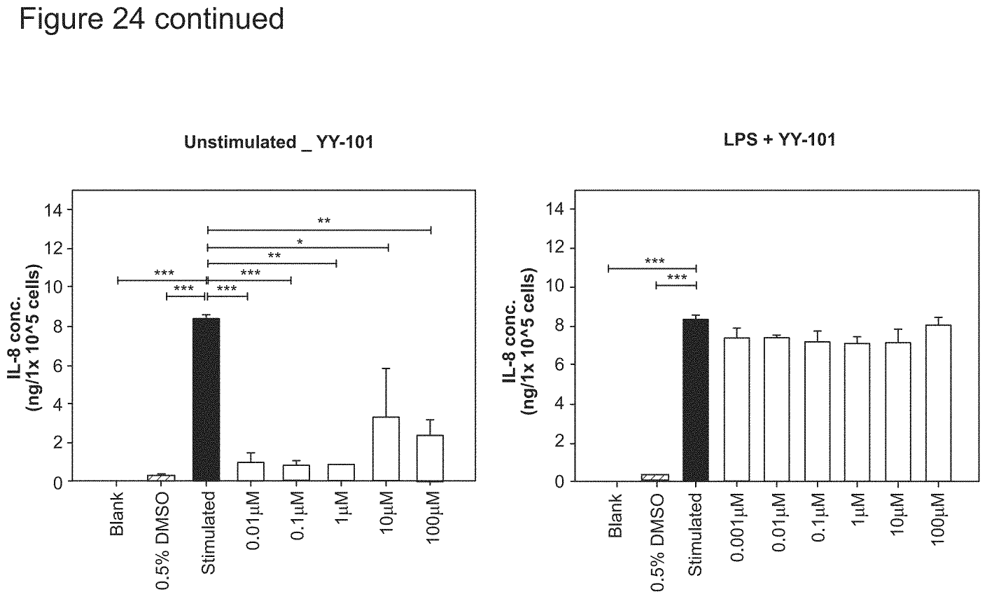

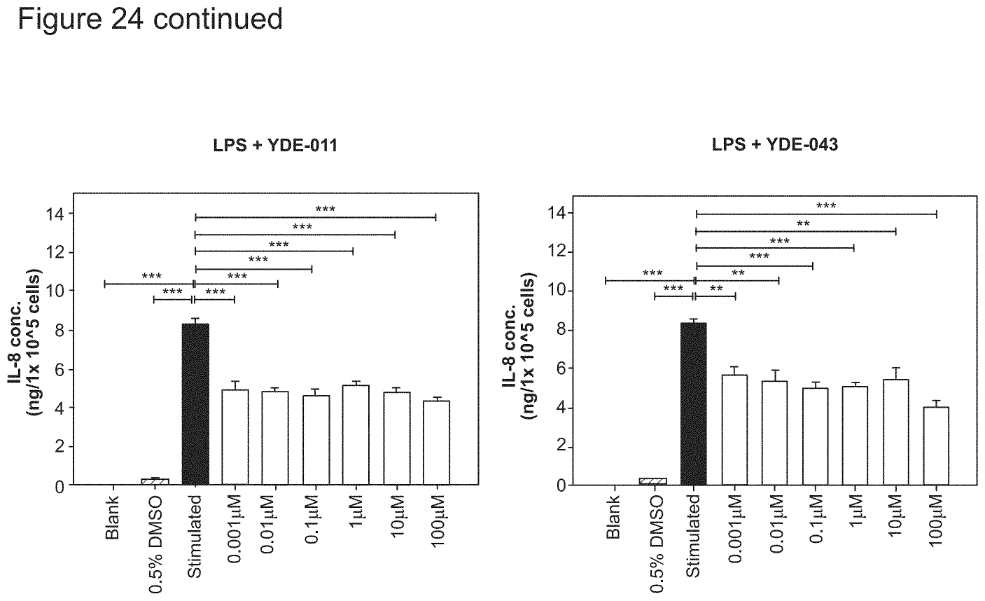

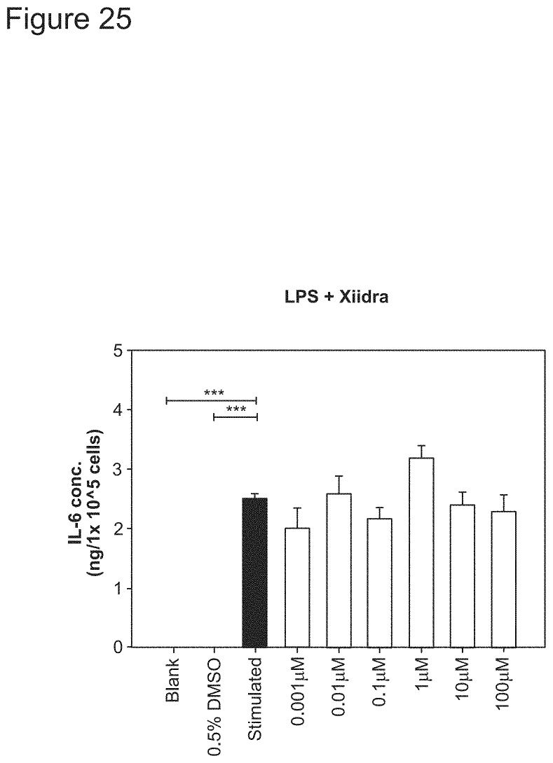

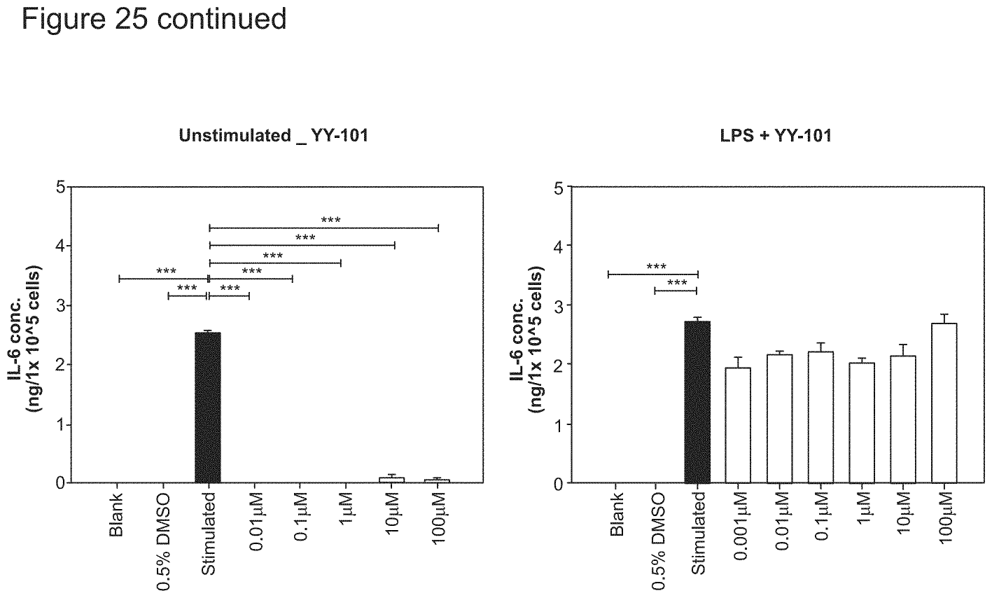

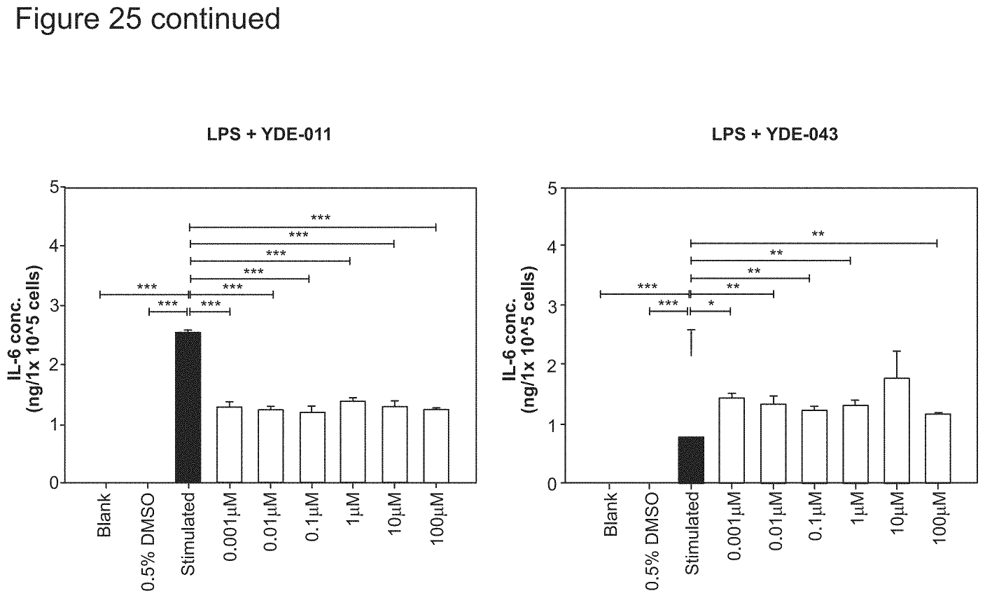

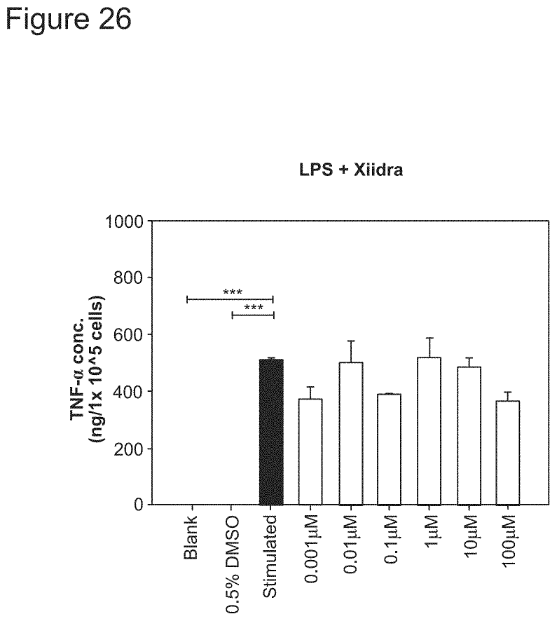

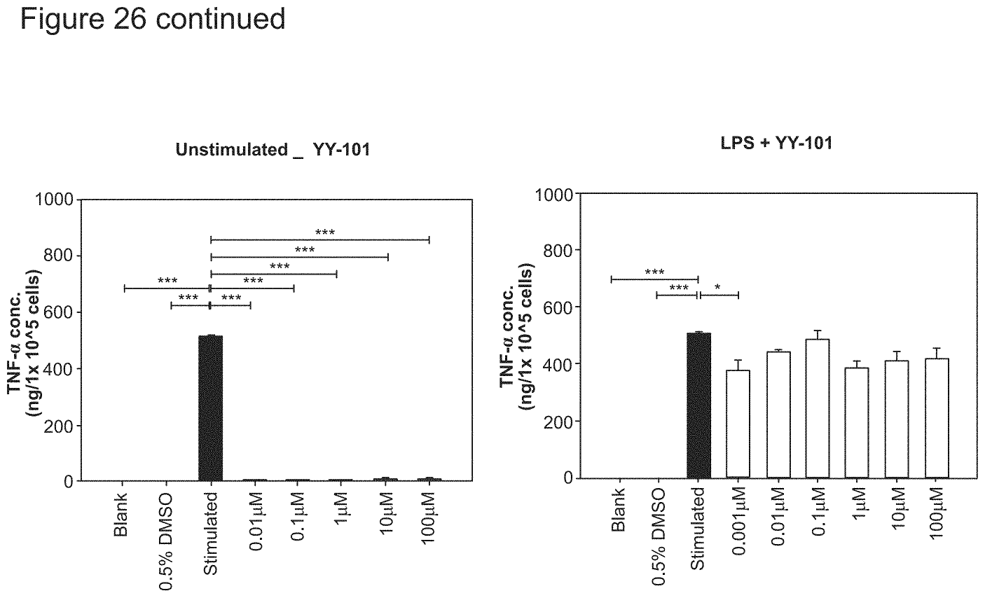

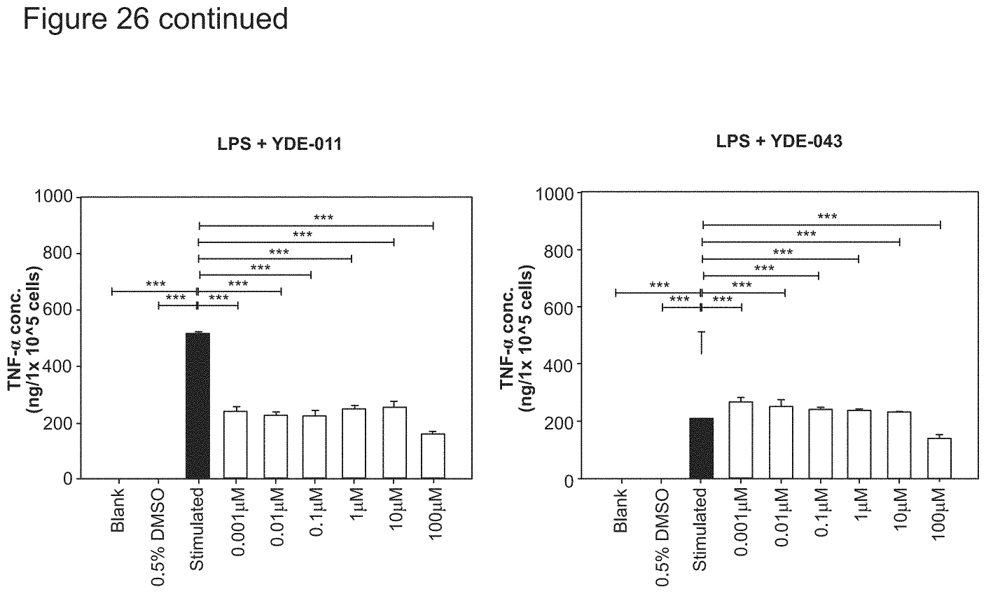

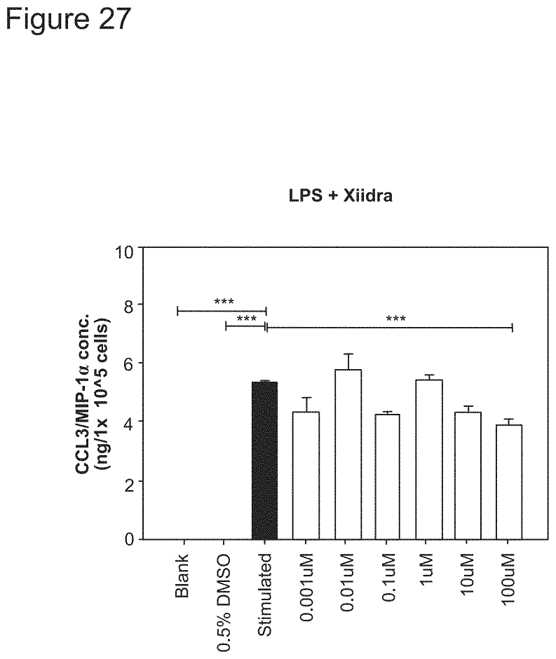

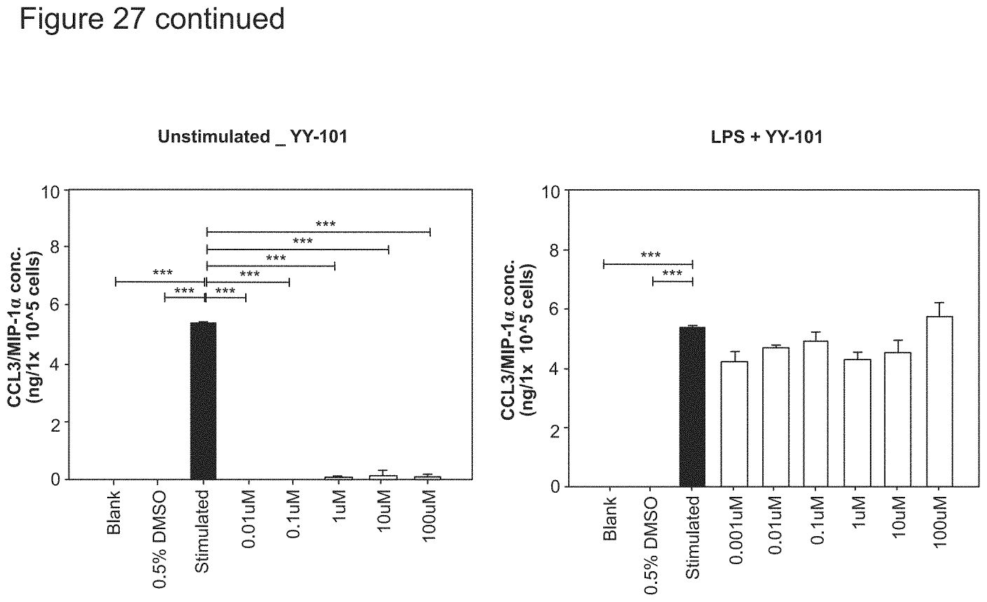

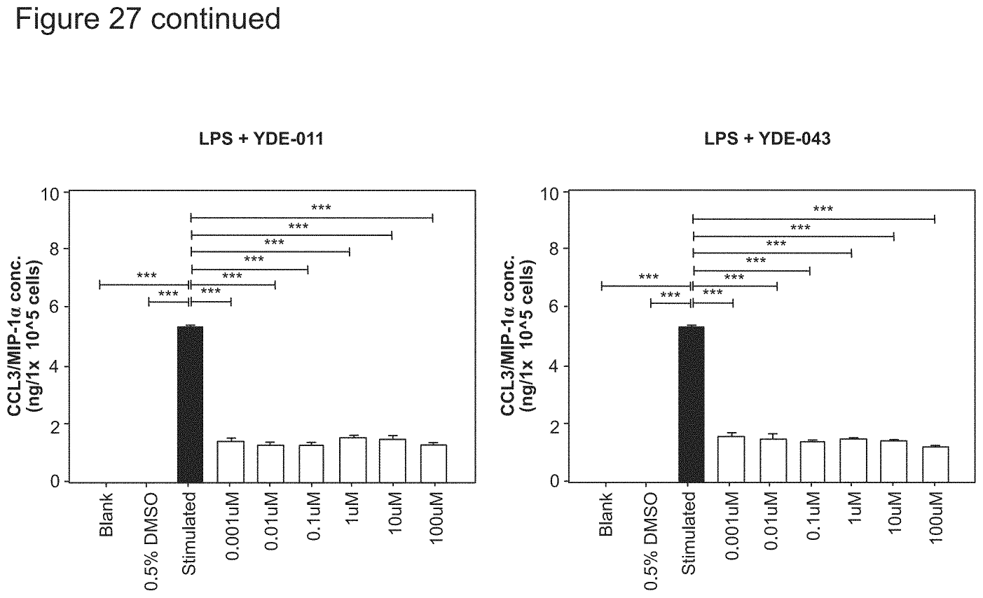

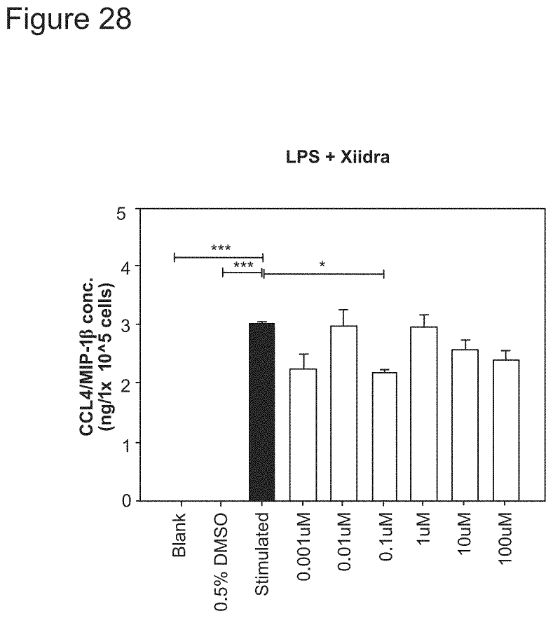

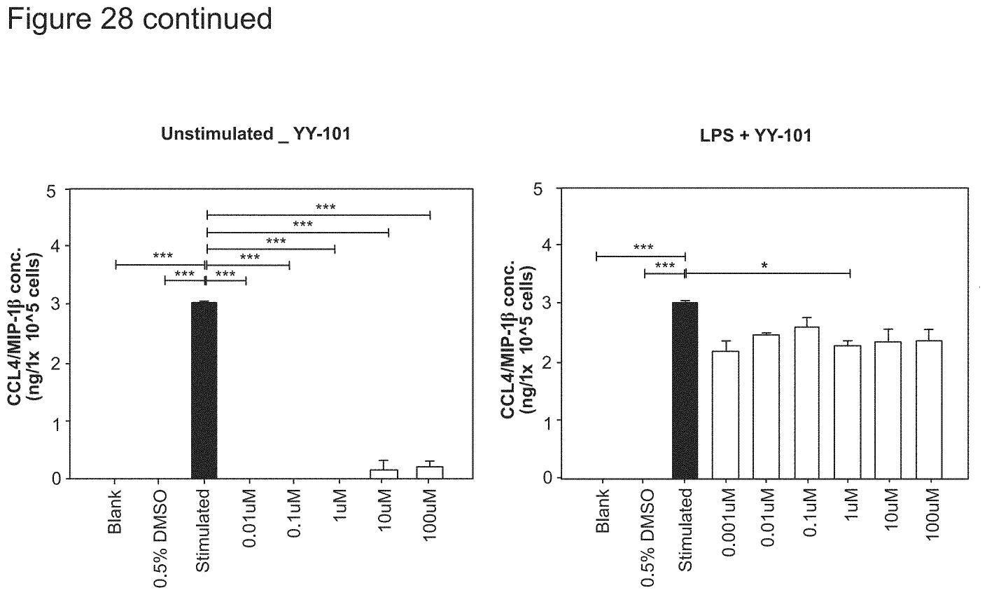

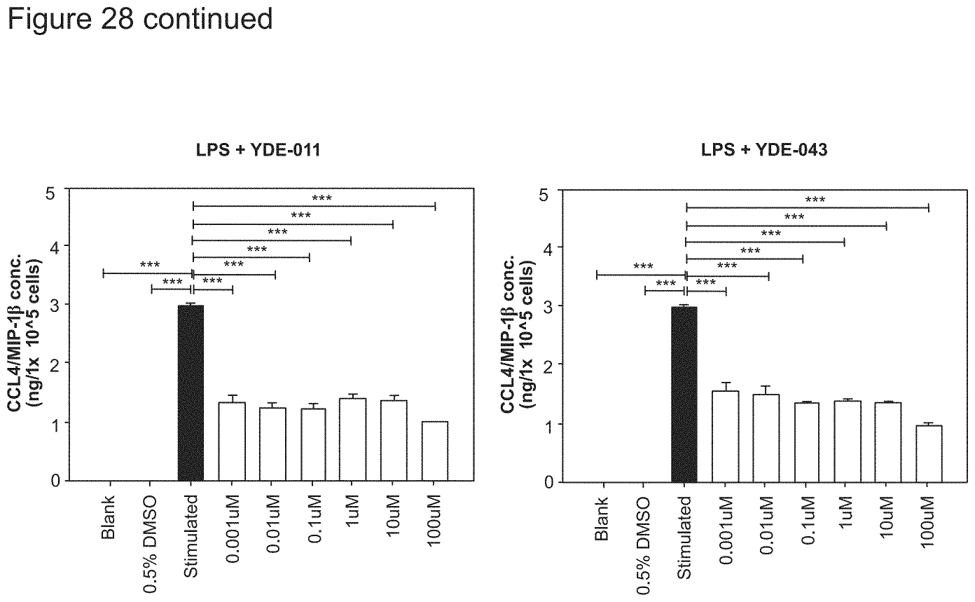

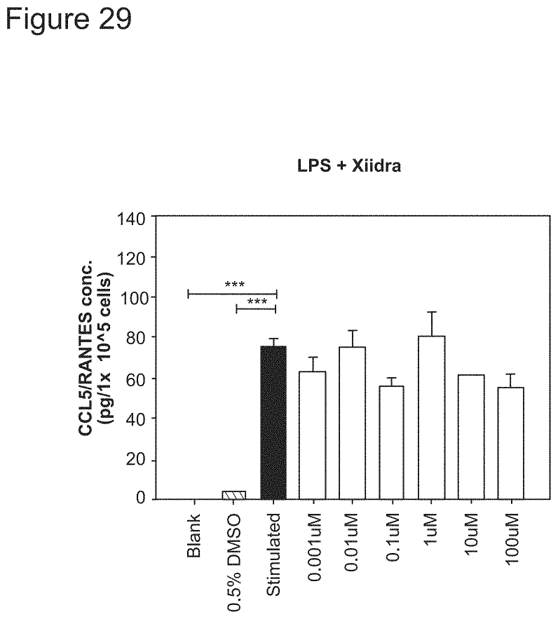

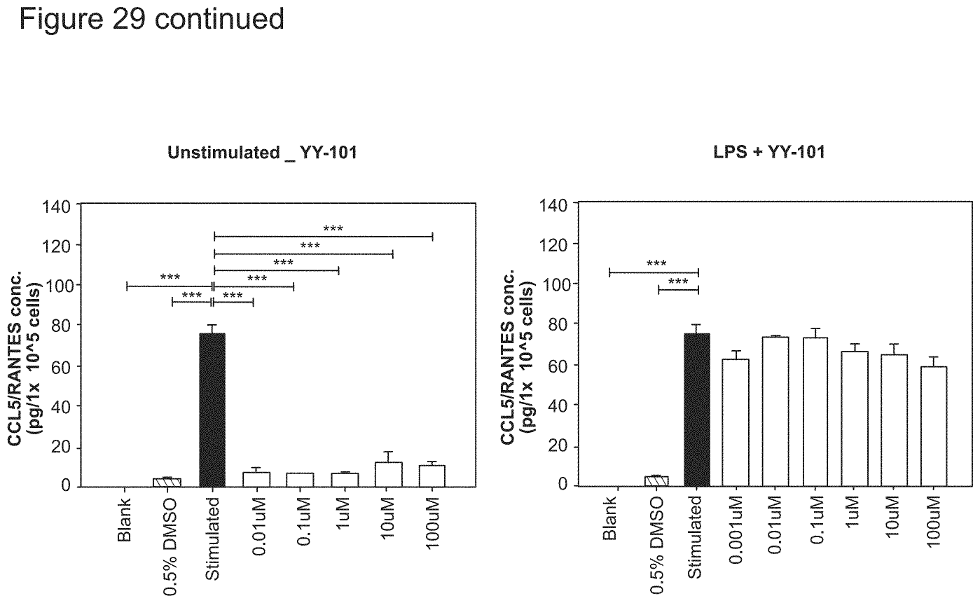

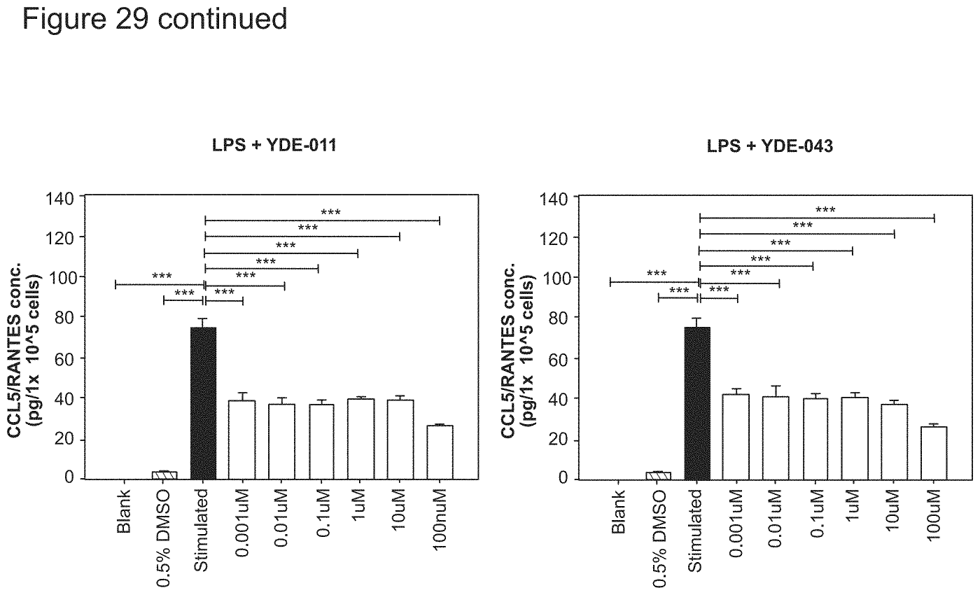

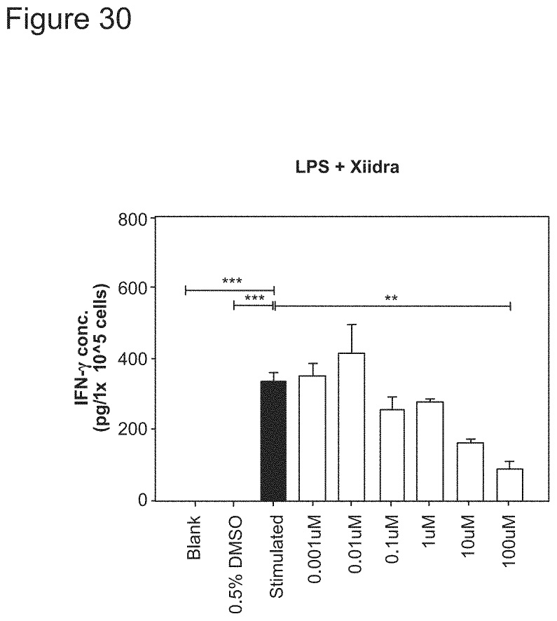

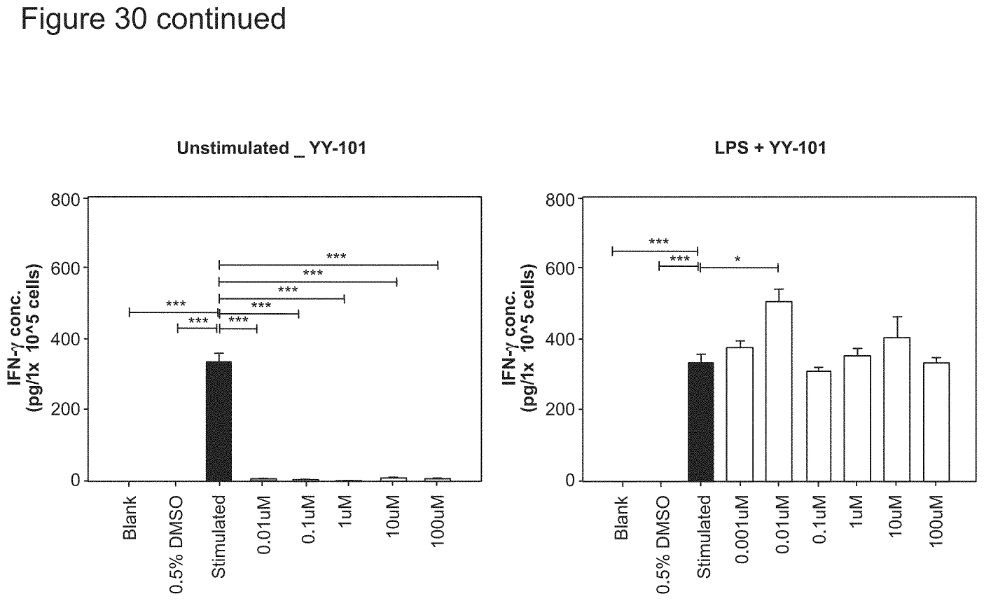

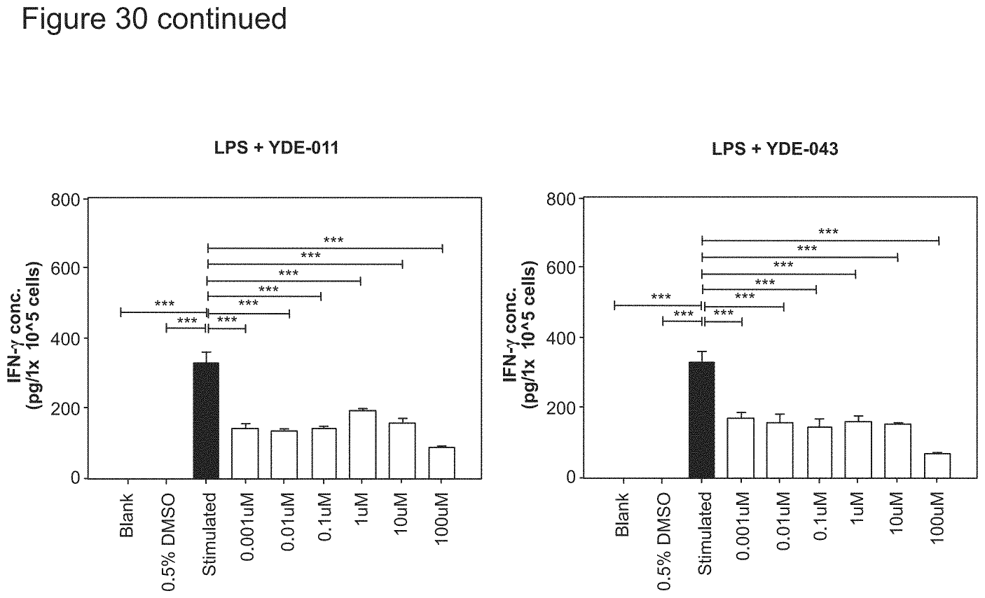

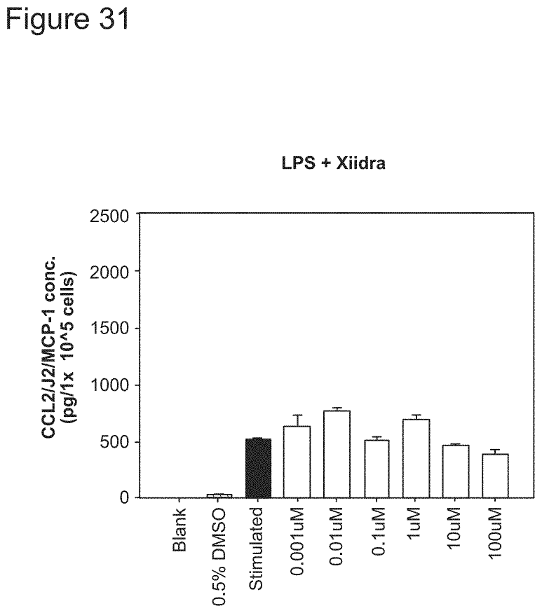

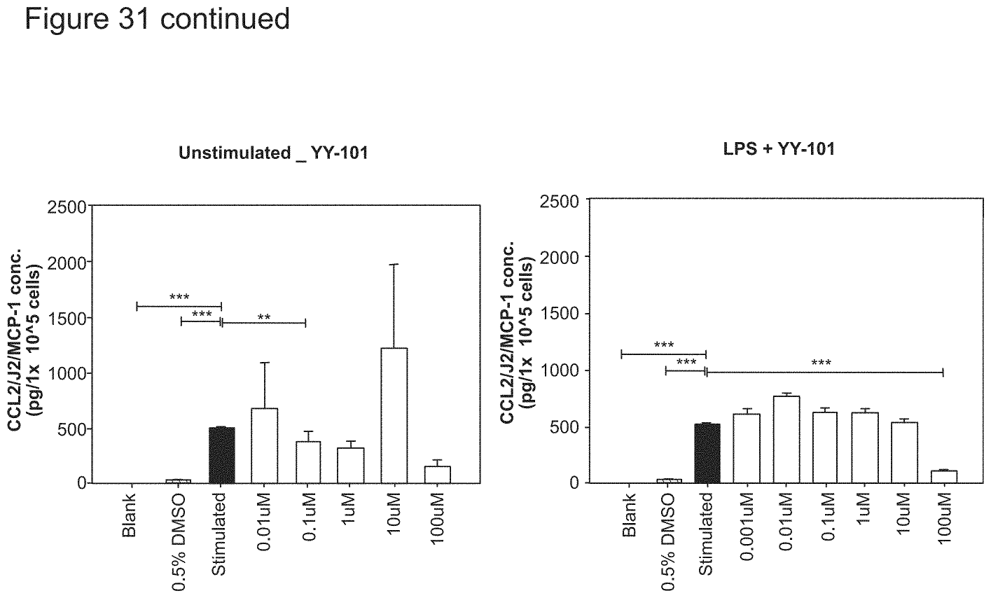

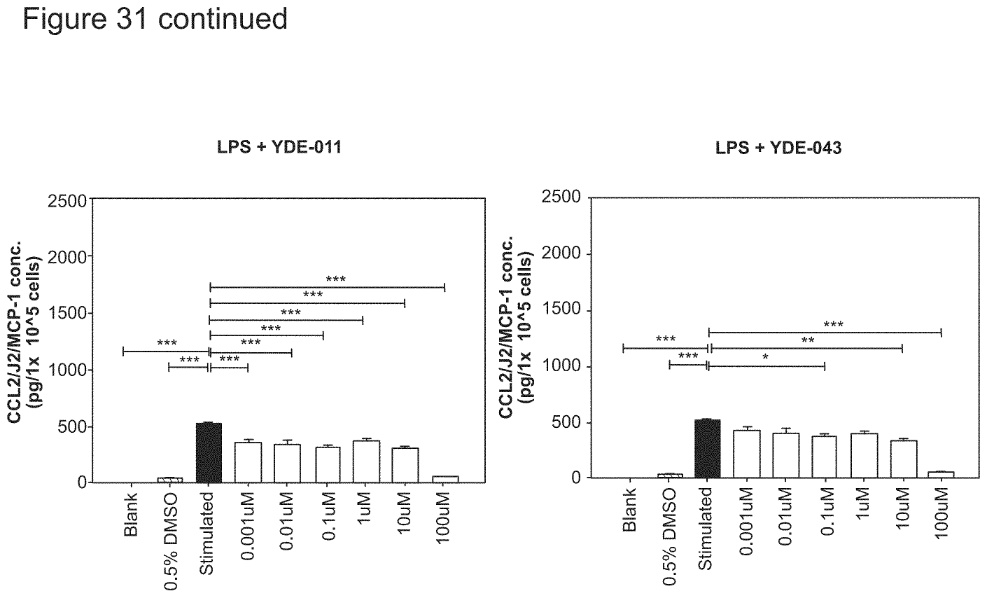

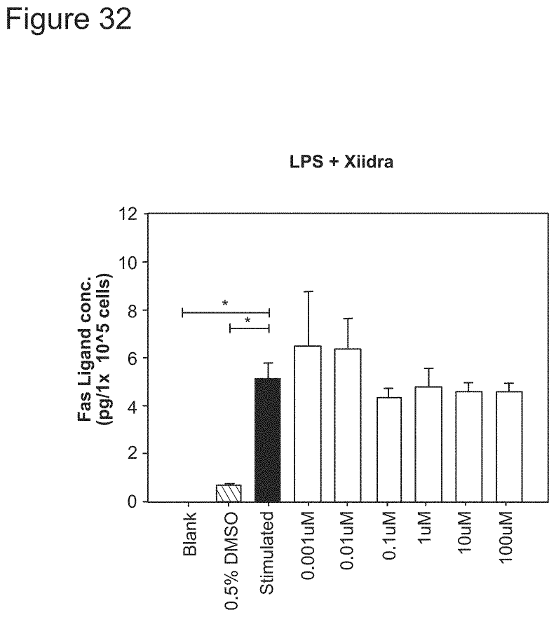

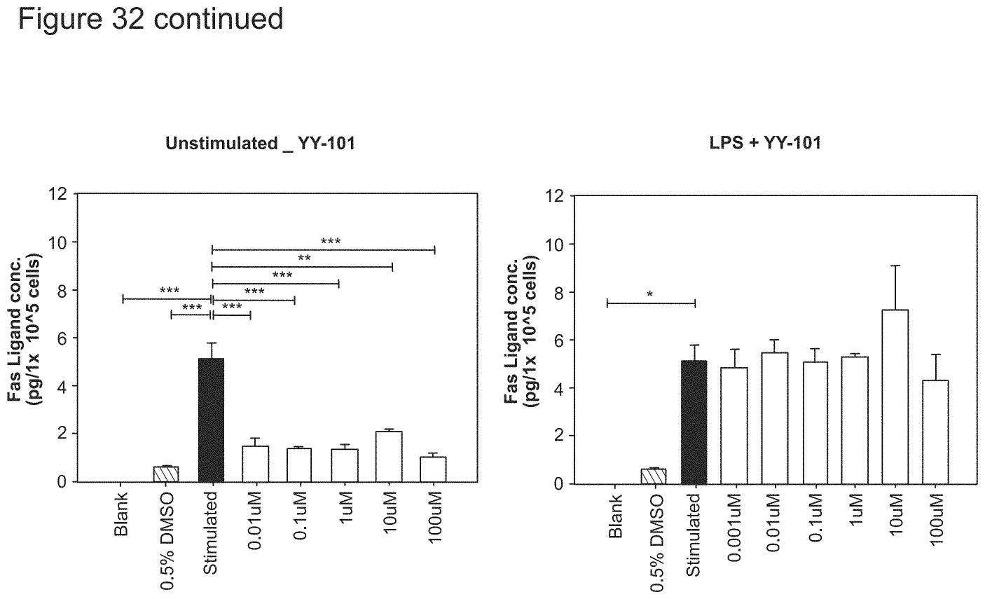

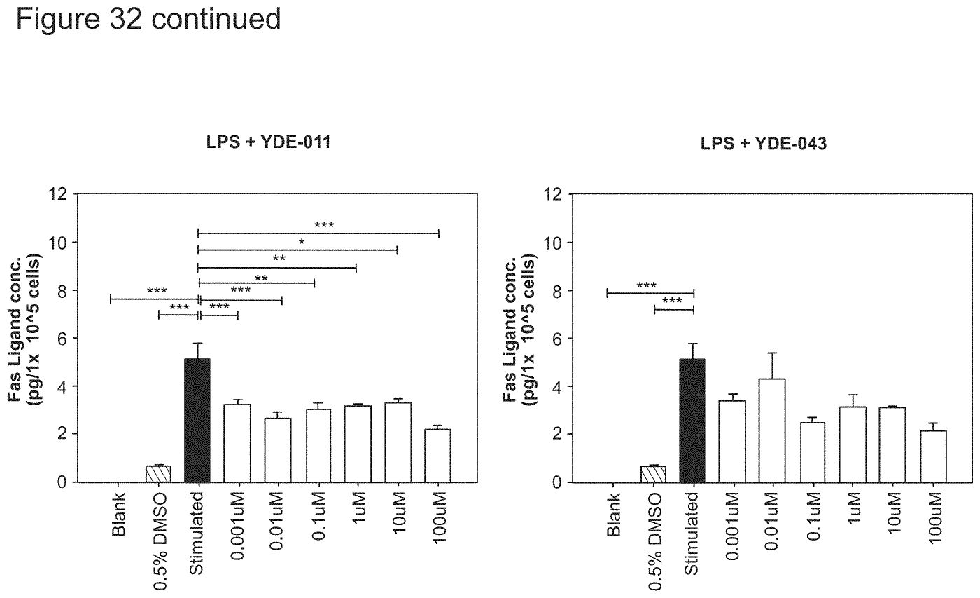

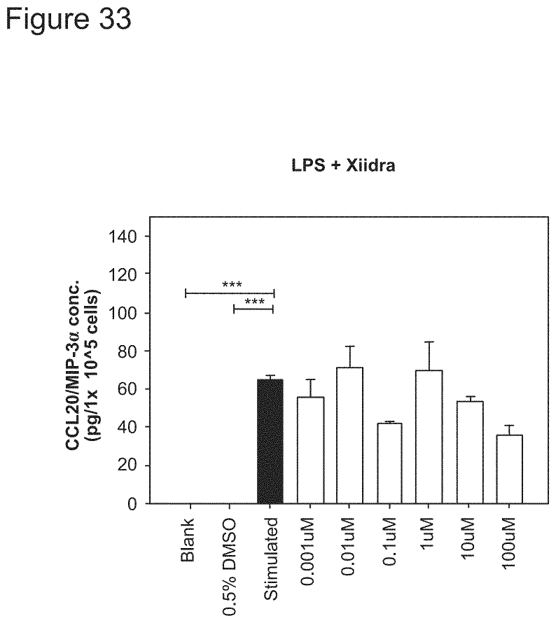

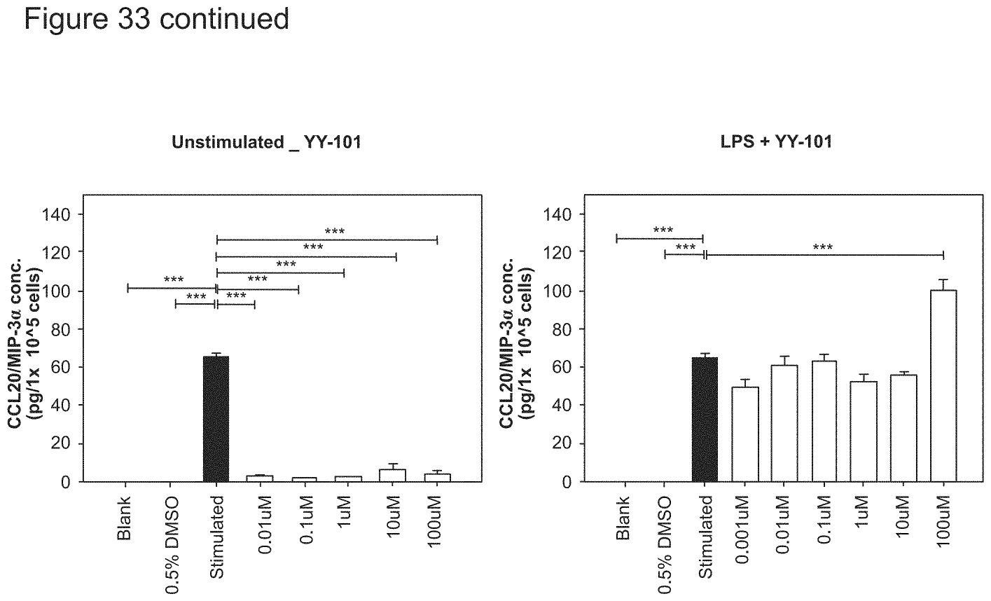

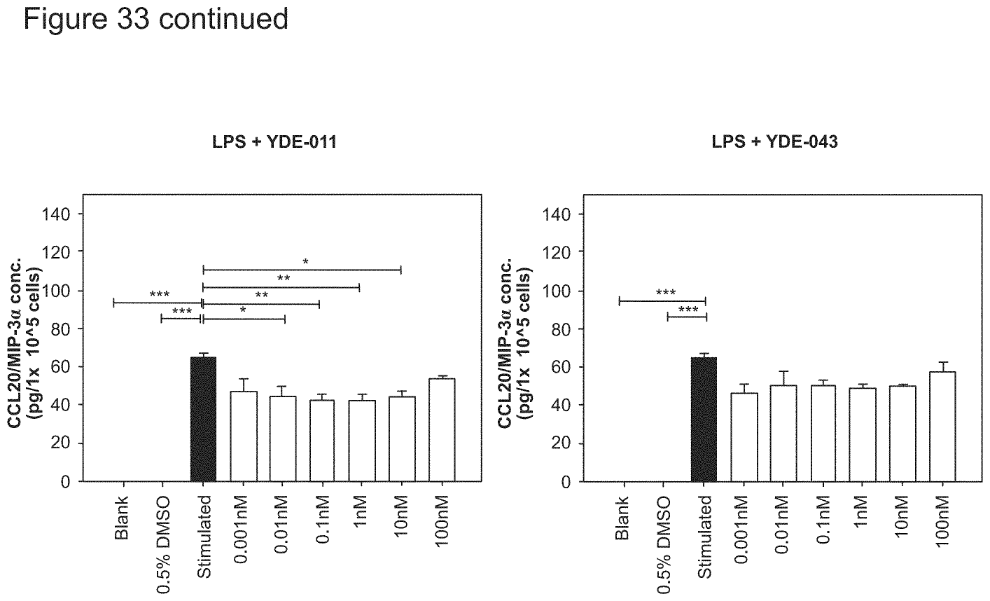

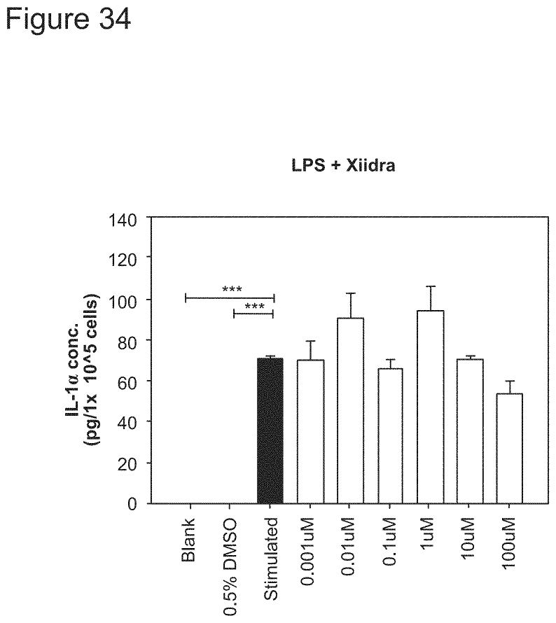

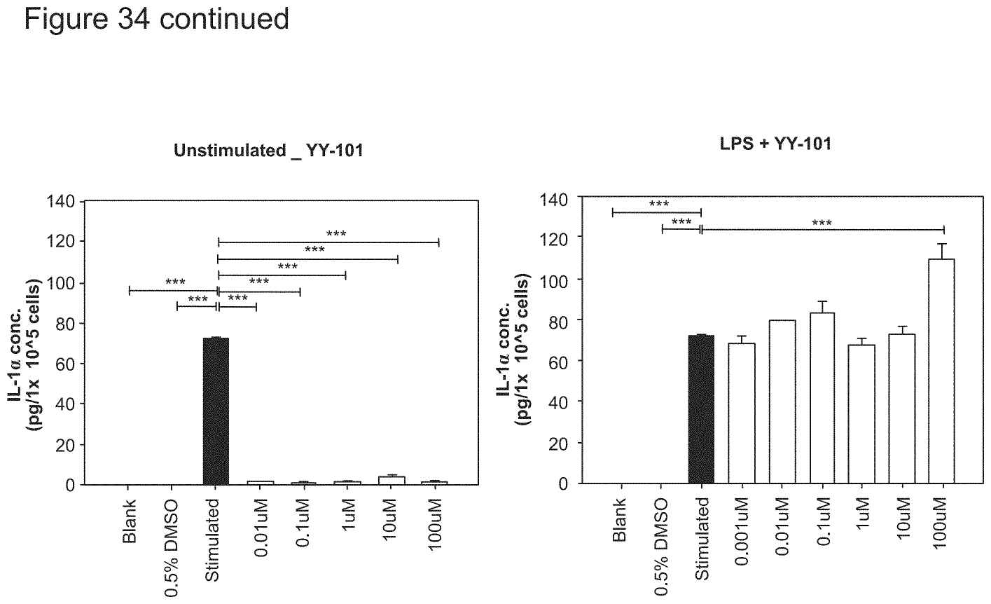

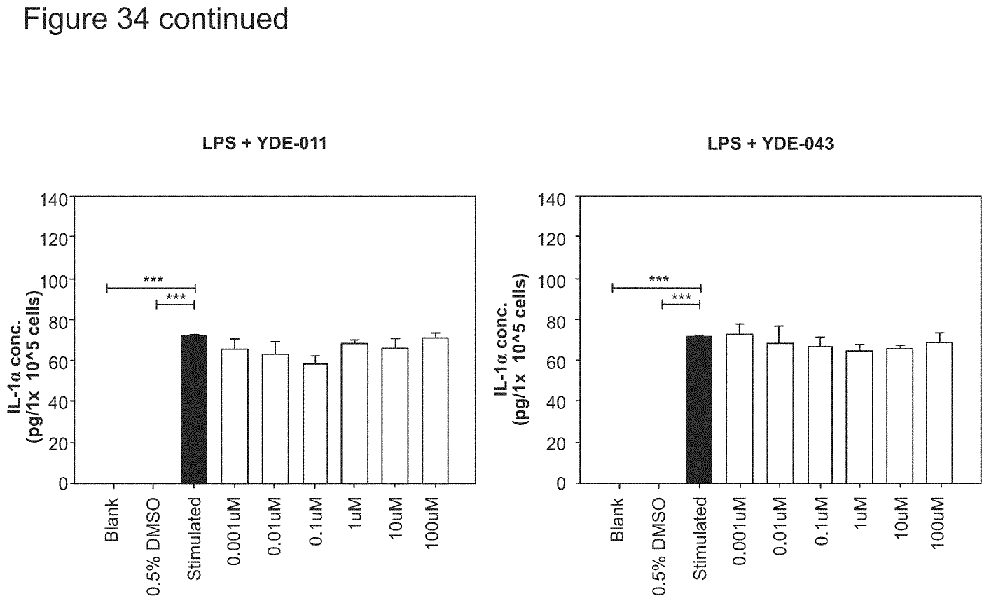

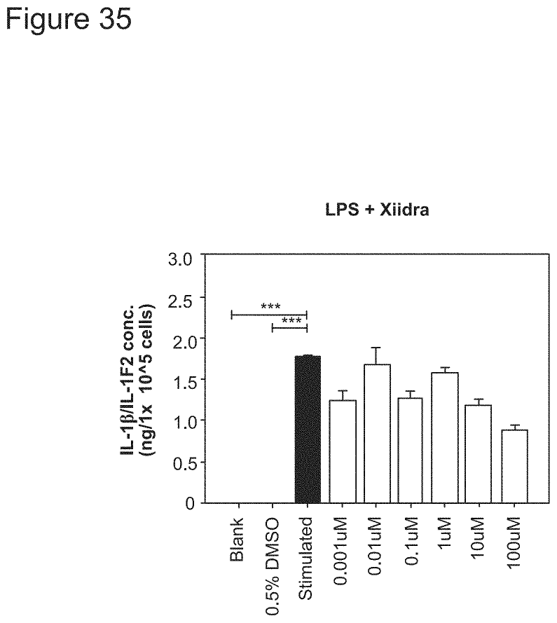

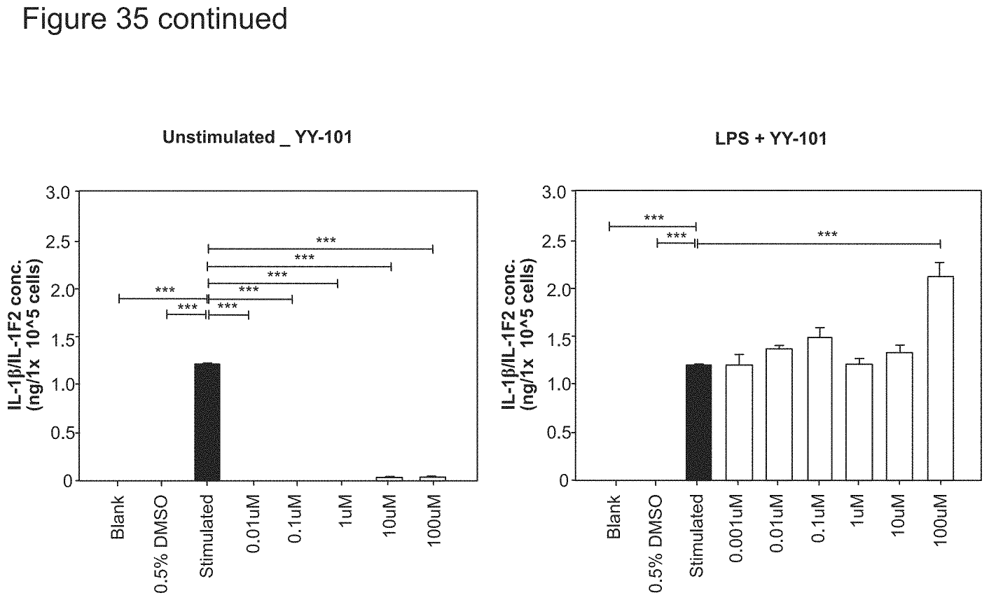

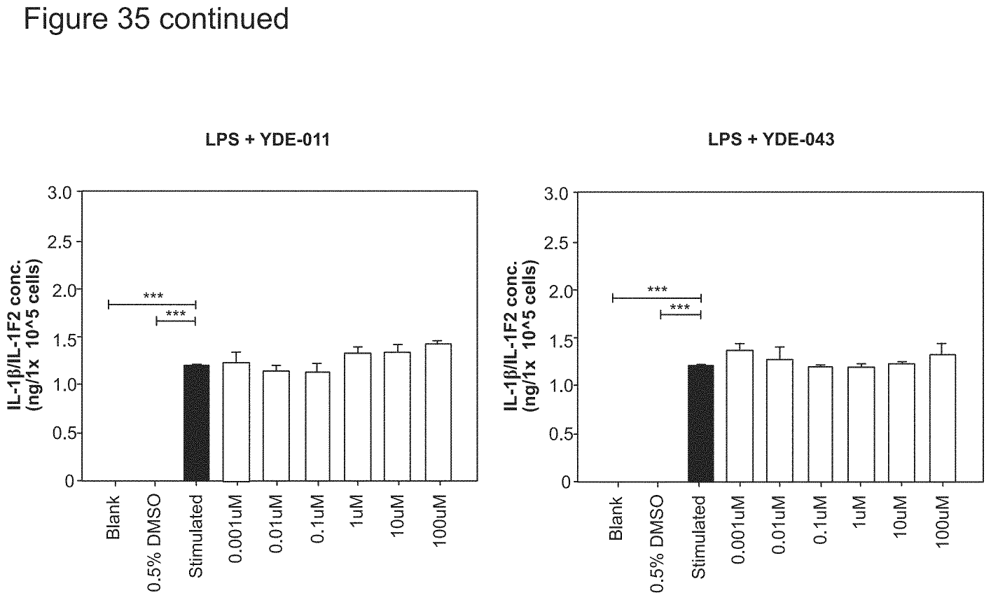

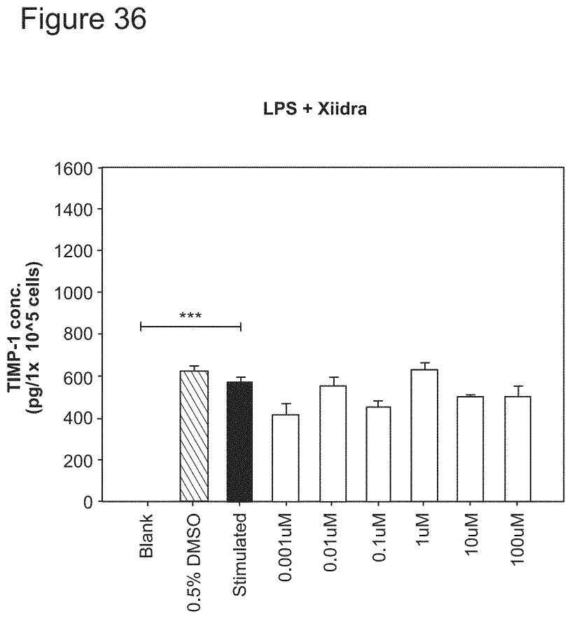

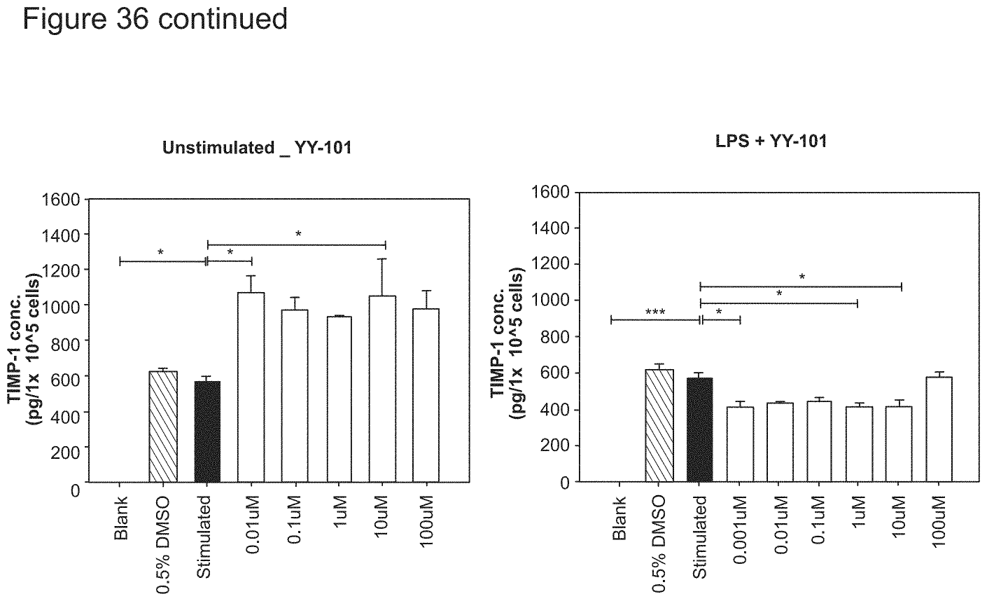

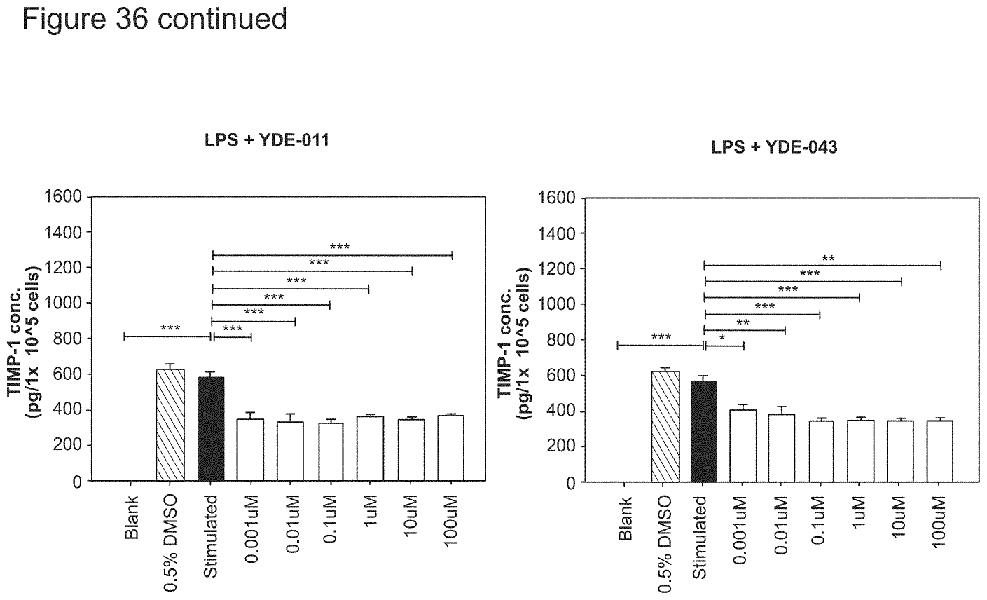

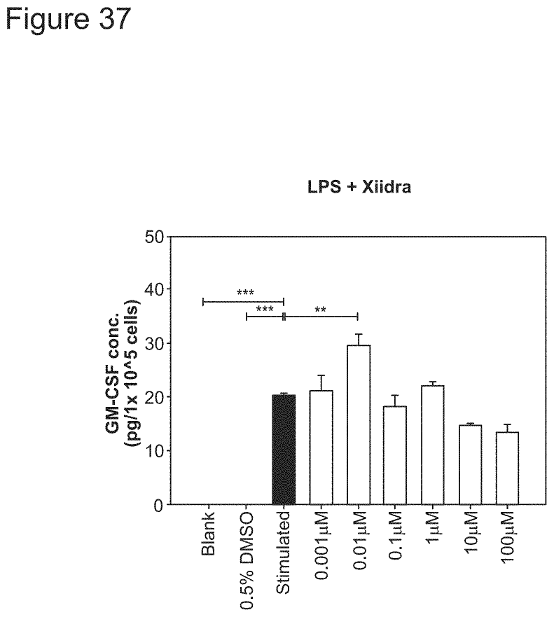

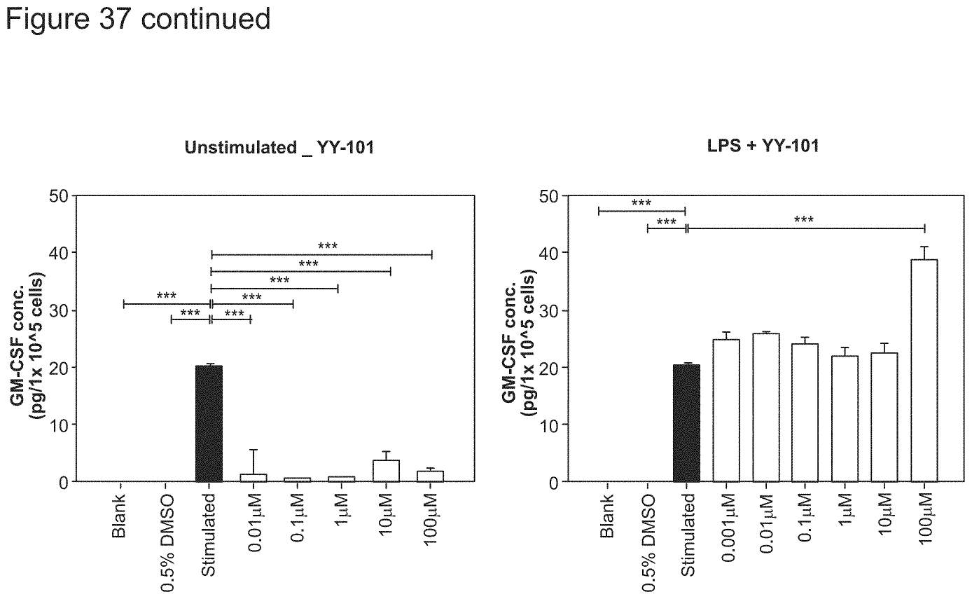

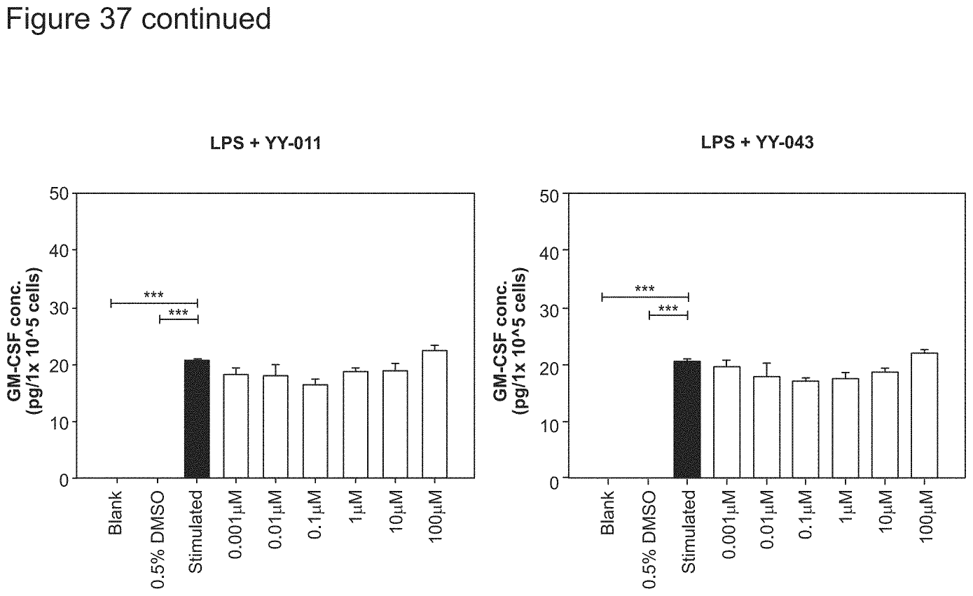

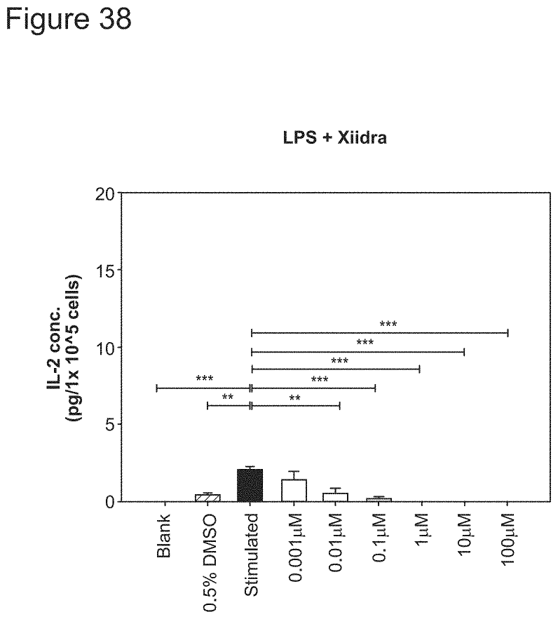

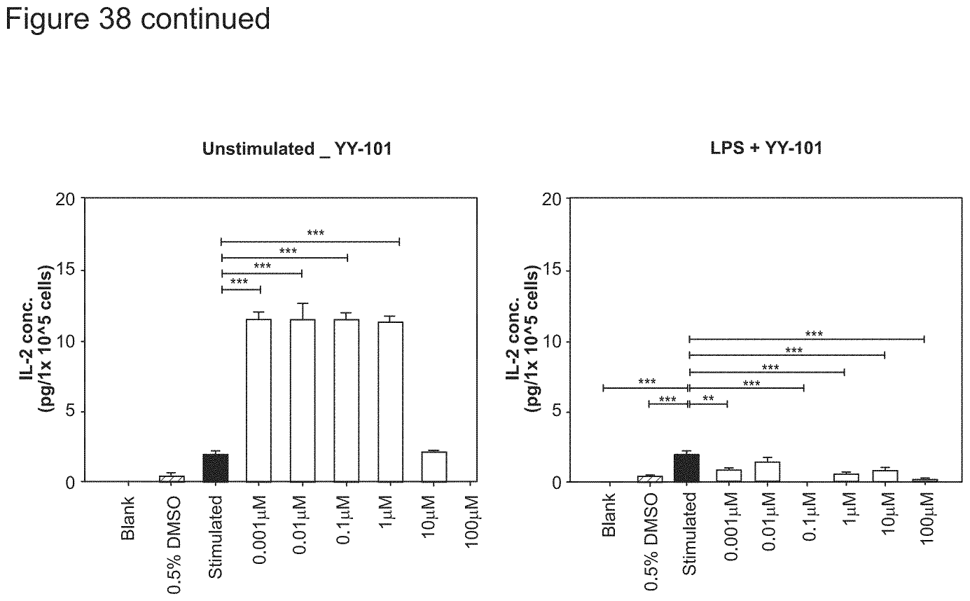

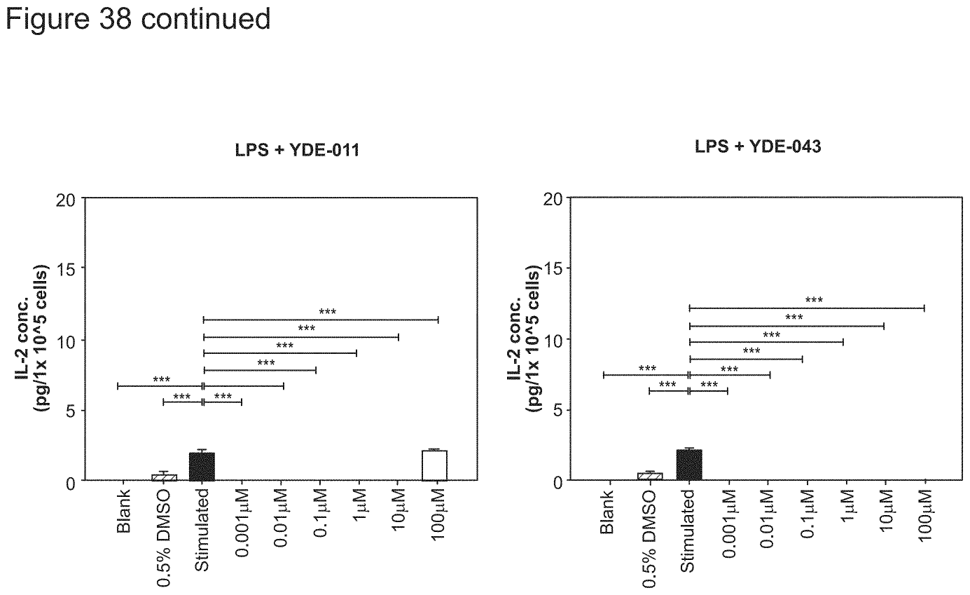

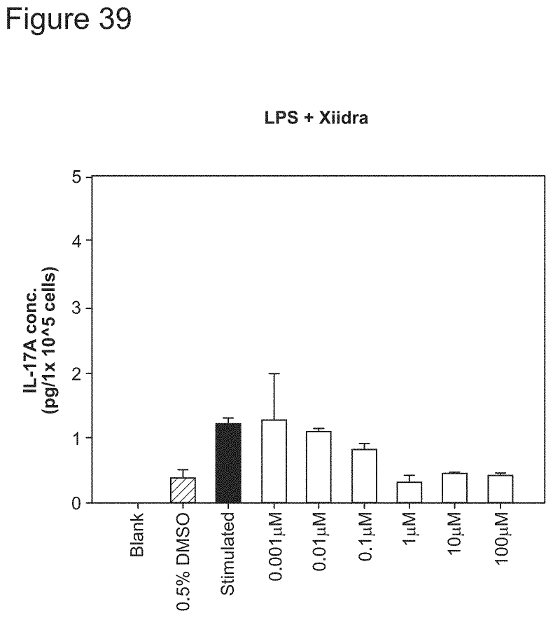

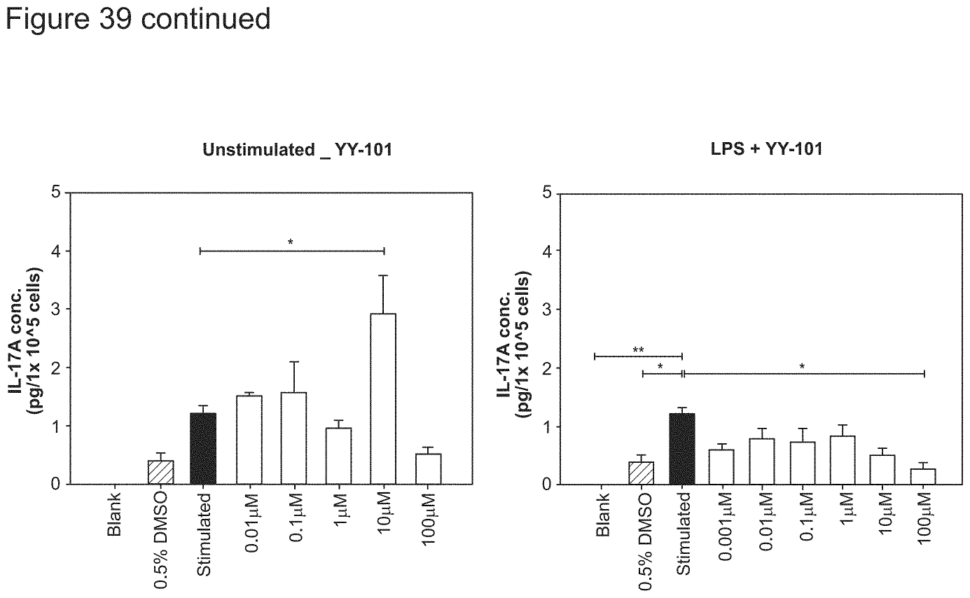

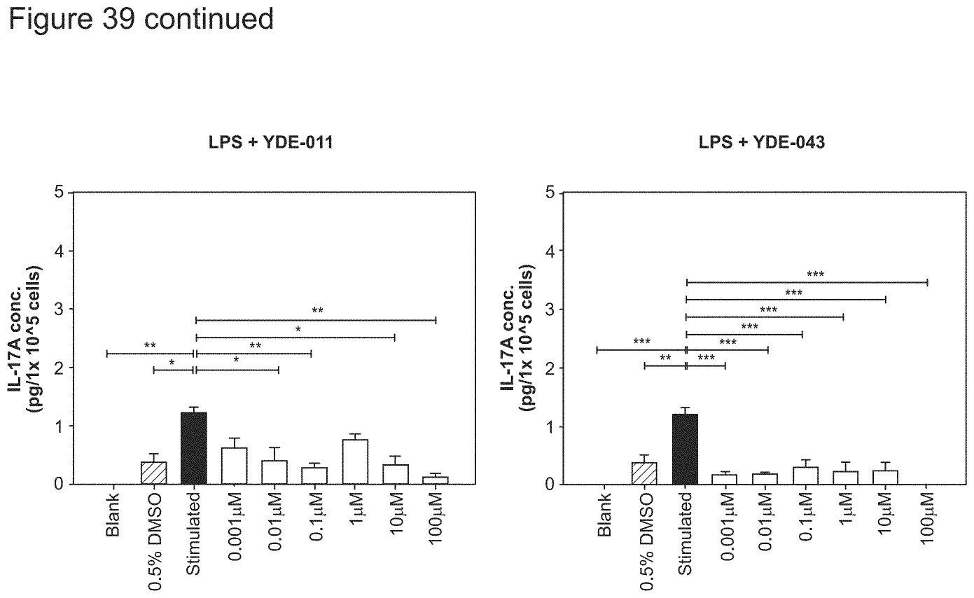

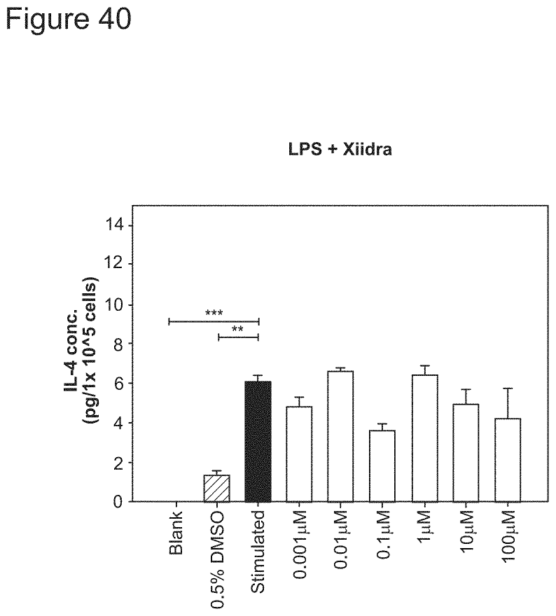

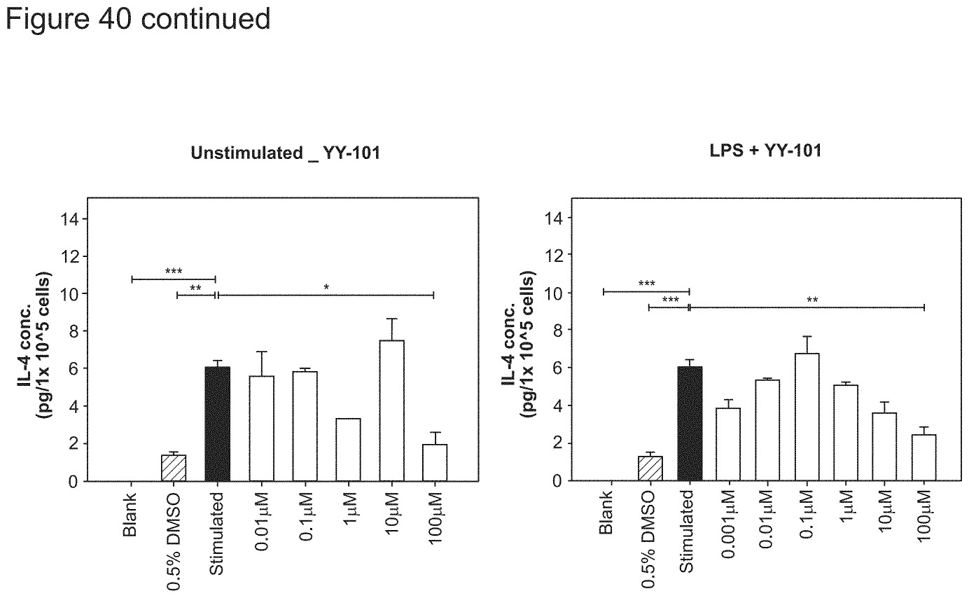

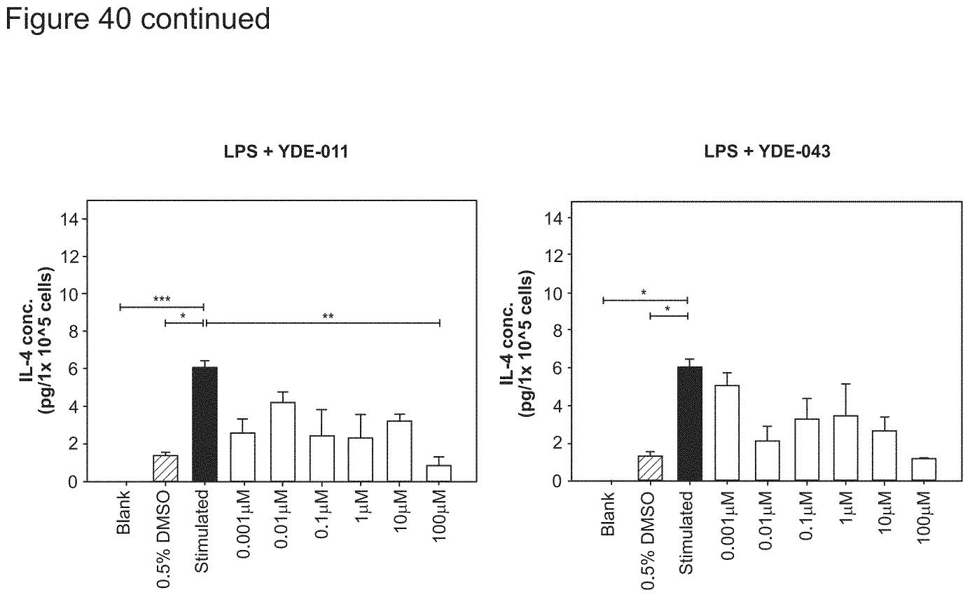

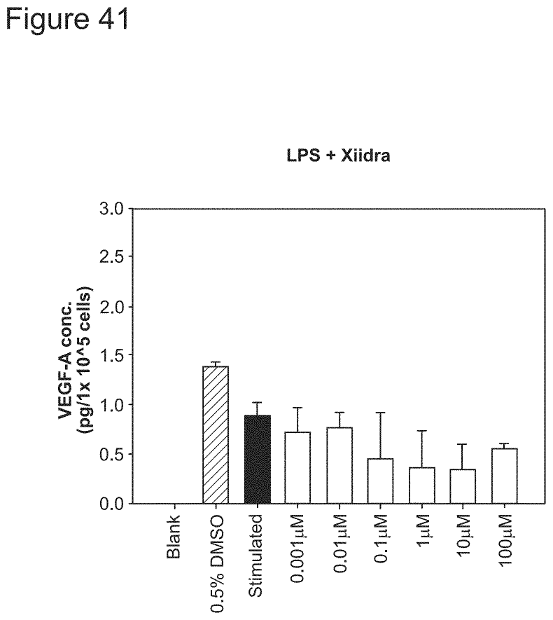

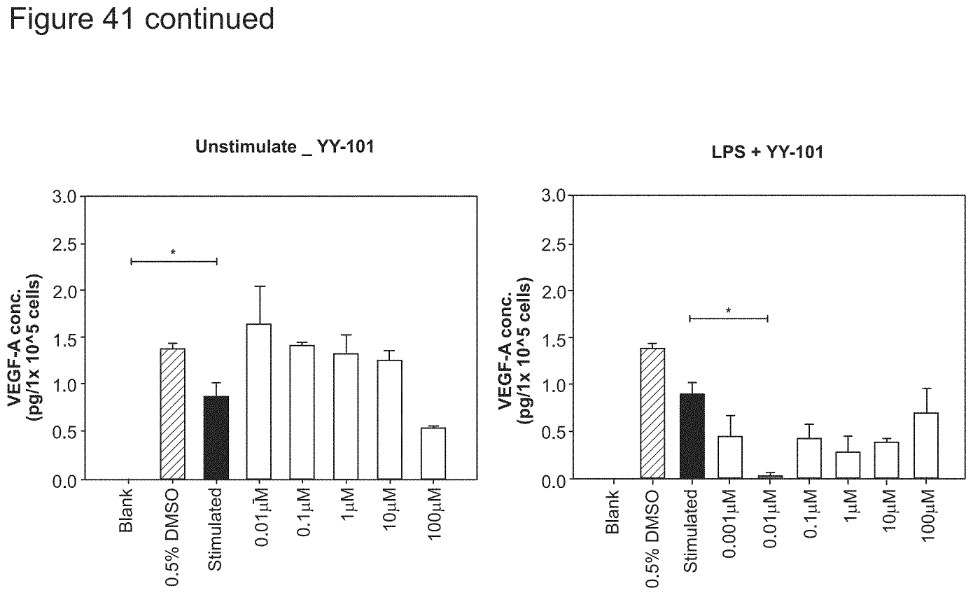

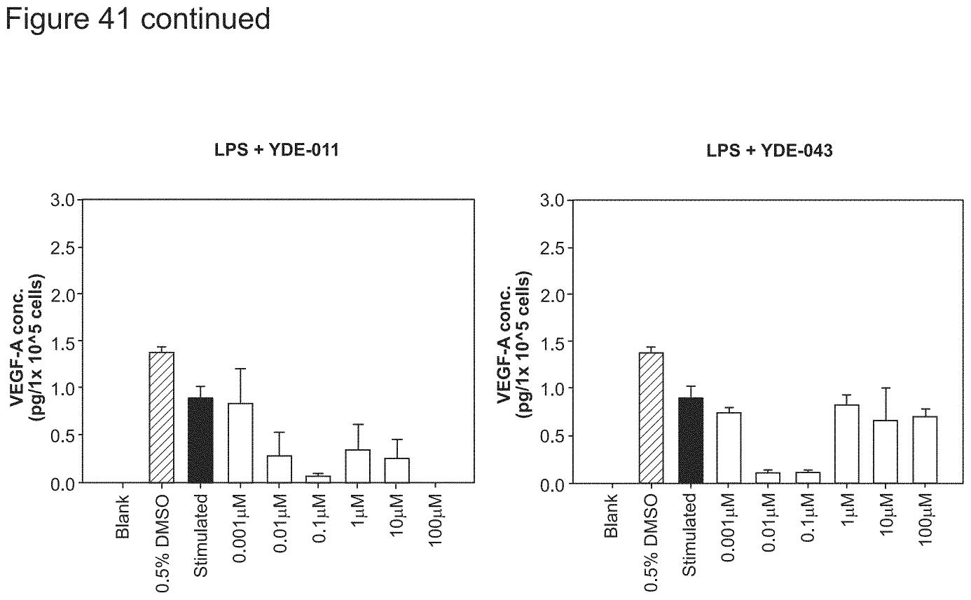

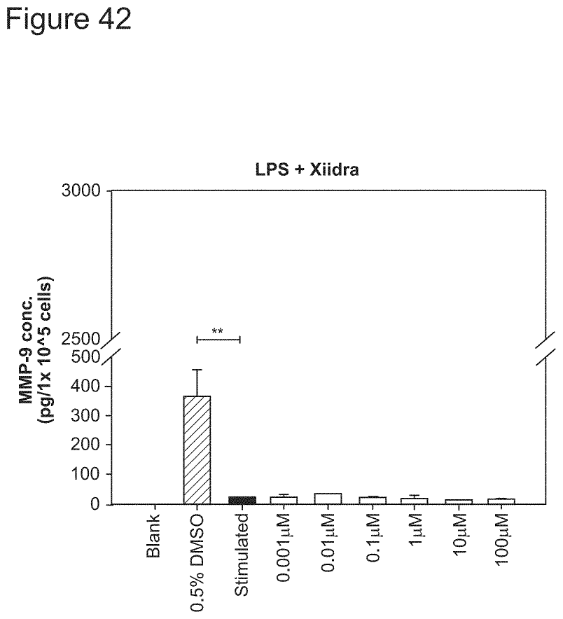

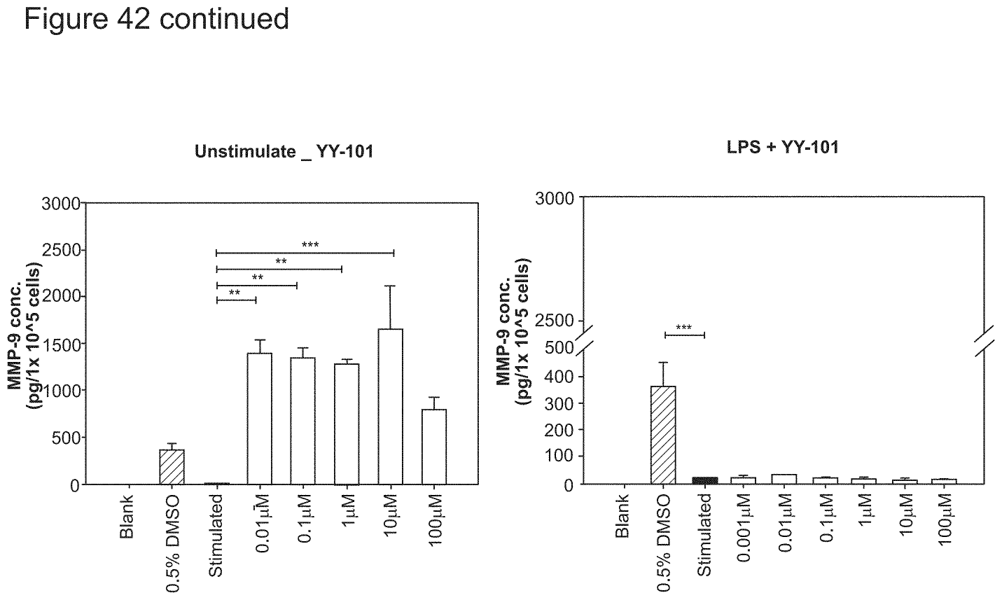

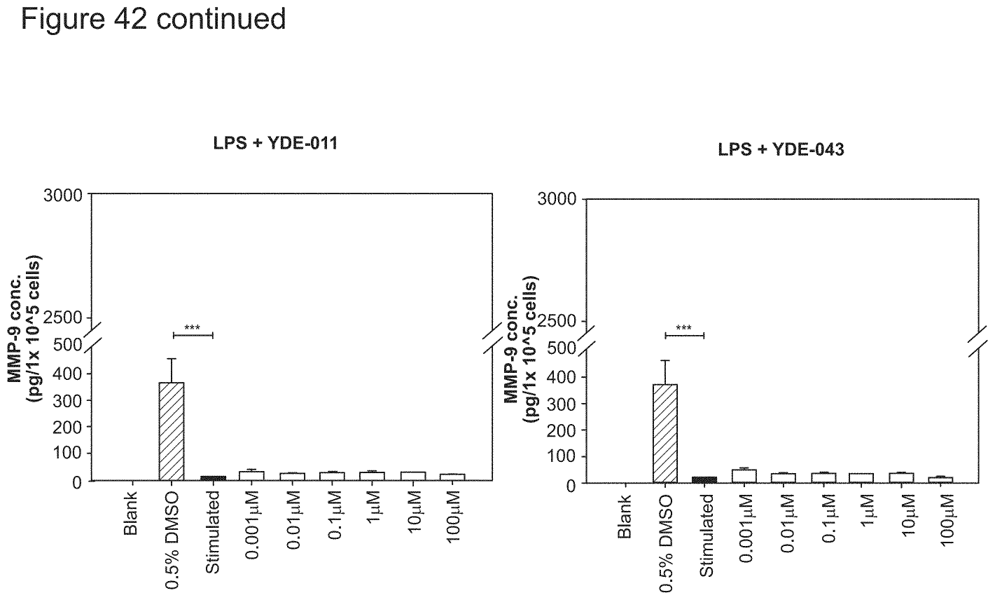

Results are shown in FIGS. 23-42. hPBMCs stimulated by LPS at 5 ug/mL for 24 hrs were co-treated with test and reference compounds with various concentrations and from which cytokine release was measured. Among 30 cytokines and chemokines evaluated, similar to the previous study (NS-Y0119 1st main test), all of the compounds, YY-101, YDE-011 and YDE-043 effectively and potently reduced LPS-induced various pro-inflammatory cytokines and chemokines in human PBMCs at as low as 1 nM. Among the compounds, both YDE-011 and YDE-043 significantly reduced various pro-inflammatory cytokines and chemokines such as TL-6, IL-8, IL-10, TNF-α, IFN-gamma, CCL2, CCL4, CCL5, CCL20, Fas, TIMP-1 more than 50% and CCL-3 over 70%. Interestingly, the compound, YY-101 didn't affect the basal levels of most of cytokine/chemokine production or the viability of hPBMCs.

Human PBMCs were stimulated by LPS at 5 ug/mL for 24 hrs and co-treated with vehicle (0.5% DMSO), test articles and reference compound, Xiidra® at various concentrations (100 uM to 1 nM). After 24 hr treatment, aliquots of hPBMC media were extracted, diluted in 1:2, 1:10 and 1:200, and measured for cytokine levels using a Luminex multiplex system. Additional aliquots were extracted to count the number of total and live PBMCs.

hPBMC Cell Counts with or without LPS and Test Articles

A range of the total and live cells after 24-hour treatment was between 5.8-13.0×10⁵ and 4.6-7.2×10⁵ cells per well (1 mL), respectively. The percentage of live over the total cells was between 54-89.5%, of which the majority falls within 7080% range (Table 1). Although some variability among groups exists, when compared to each treatment groups, little or no difference in the total and live cells or viability % was observed in any groups. In other words, none of LPS alone or YY-101 alone, LPS+Xiidra® LPS+YY-101, LPS+YDE-011, or LPS+YDE-043 consistently affected the total and live number of PBMCs up to 100 μM. Neither did the percent viability.

### Multi-Cytokine Assessment in PBMCs Stimulated by LPS

Among 30 cytokines & chemokines evaluated, 15 of 30 were detected within the standard curve range, 5 below the range and 10 were below the level of detection & quantification even though the sample dilution was minimized (Table 2).

Among those 15 detectable cytokines/chemokines, similar to the previous study (NS-Y0119 1^{st} main test), all of the compounds, YY-101, YDE-011 and YDE-043, effectively and potently reduced LPS-induced various pro-inflammatory cytokines and chemokines in human PBMCs at as low as 1 nM. Among the compounds, both YDE-011 and YDE-043 significantly reduced various pro-inflammatory cytokines and chemokines such as IL-6, IL-8, IL-10, TNF-α, IFN-gamma, CCL2, CCL4, CCL5, CCL20, Fas, TIMP-1 more than 50% and CCL-3 over 70%. YY-101 also significantly reduced LPS-induced various cytokines and chemokines (IL-10, TNF-alpha, CCL4) in human PBMCs at as low as 1 nM.

CCL3 is a chemokine ligand 3 known as macrophage inflammatory protein 1-alpha (MIP-1-alpha), which is involved in the acute inflammatory state in the recruitment and activation of leukocytes through binding to the receptors CCR1, CCR4 and CCR5. CCL3 has known to interact with CCL4 and attracts macrophages, monocytes and neutrophils. Interestingly, CCL3 concentrations were significantly increased in Sjogren's syndrome patients and CCL3 and CCL4 levels correlated significantly with basal tear secretion, tear clearance rate, keratoepitheliopathy score, and goblet cell density. The level correlates with various tear film and ocular surface parameters. (Choi et al., Curr Eye Res. 2012 37:12-7).

Although the detection level was low, all of the compounds potently reduced LPS-induced IL2, IL-4 and IL-17A levels in a dose-dependent manner. In contrast, YY-101 also further increased LPS-induced IL-1a, IL-I3, CCL20 and GM-CSF levels at 100.iM, which may due to the YY-101 mediated cellular toxicity. However, it is unlikely as YY-101 at 100.iM didn't affect any viability or the total number of PBMCs (Table 1). Similar to the previous finding, YY-101, even up to 100.iM didn't affect the basal levels of cytokine production except TIMP-1.

When the reference compound, Xiidra® was tested at higher concentrations than the previous study, it began to reduce some of cytokines induced by LPS. Xiidra® significantly reduced IL-10, CCL3, CCL4, IFN-γ.

TABLE 24
PBMC Cell Counts with and without LPS and test articles.

| | Total cell | Live cell | Viability |
|---|---|---|---|
| | (10⁵/ml) | (10⁵/ml) | (%) |
| PBMC + 0.5% DMSO | 8.2 ± 0.4 | 7.2 ± 0.3 | 87.3 ± 0.9 |
| PBMC + LPS(5 ug/ml) | 6.8 ± . | 5.8 ± 0.2 | 85.0 ± 0.6 |
| PBMC + YY101 0.01 uM | 5.9 ± 0.4 | 5.0 ± 0.2 | 85.0 ± 2.0 |
| PBMC + YY101 0.1 uM | 6.1 ± 0.8 | 5.1 ± 0.2 | 84.0 ± 0.0 |
| PBMC + YY101 1 uM | 6.1 ± 0.2 | 5.5 ± 0.0 | 89.5 ± 2.5 |
| PBMC + YY101 10 uM | 7.4 ± 0.3 | 6.3 ± 0.2 | 86.0 ± 1.0 |
| PBMC + YY101 100 uM | 7.8 ± 0.5 | 6.7 ± 0.3 | 86.5 ± 1.5 |
| PBMC + LPS(5 ug/ml) + | 13.0 ± 3.0 | 6.9 ± 0.7 | 54.0 ± 8.0 |
| Xiidra ® 0.001 uM | | | |
| PBMC + LPS(5 ug/ml) + | 6.9 ± 0.6 | 5.5 ± 0.3 | 80.0 ± 2.0 |
| Xiidra ® 0.01 uM | | | |
| PBMC + LPS(5 ug/ml) + | 7.1 ± 0.7 | 6.2 ± 0.5 | 86.5 ± 1.5 |
| Xiidra ® 0.1 uM | | | |
| PBMC + LPS(5 ug/ml) + | 5.8 ± 0.3 | 4.6 ± 0.2 | 80.5 ± 0.5 |
| Xiidra ® 1 uM | | | |
| PBMC + LPS(5 ug/ml) + | 6.9 ± 0.2 | 6.1 ± 0.3 | 89.0 ± 2.0 |
| Xiidra ® 10 uM | | | |
| PBMC + LPS(5 ug/ml) + | 7.5 ± 0.9 | 6.5 ± 0.7 | 86.5 ± 1.5 |
| Xiidra ® 100 uM | | | |
| PBMC + LPS(5 ug/ml) + | 7.7 ± 0.6 | 6.5 ± 0.4 | 84.3 ± 2.6 |
| YY-101 0.001 uM | | | |
| PBMC + LPS(5 ug/ml) + | 8.1 ± 0.6 | 6.2 ± 0.1 | 77.3 ± 5.0 |
| YY-101 0.01 uM | | | |
| PBMC + LPS(5 ug/ml) + | 7.8 ± 0.8 | 5.9 ± 0.3 | 75.7 ± 4.7 |
| YY-101 0.1 uM | | | |
| PBMC + LPS(5 ug/ml) + | 9.8 ± 0.6 | 6.5 ± 0.3 | 66.7 ± 5.8 |
| YY-101 1 uM | | | |
| PBMC + LPS(5 ug/ml) + | 9.1 ± 1.1 | 6.7 ± 0.5 | 73.7 ± 4.8 |
| YY-101 10 uM | | | |
| PBMC + LPS(5 ug/ml) + | 7.8 ± 1.0 | 5.8 ± 0.4 | 76.7 ± 5.2 |
| YY-101 100 uM | | | |
| PBMC + LPS(5 ug/ml) + | 6.3 ± 0.2 | 5.4 ± 0.4 | 85.0 ± 5.1 |
| YDE-011 0.001 uM | | | |
| PBMC + LPS(5 ug/ml) + | 7.3 ± 0.4 | 5.9 ± 0.4 | 81.0 ± 1.2 |
| YDE-011 0.01 uM | | | |
| PBMC + LPS(5 ug/ml) + | 7.1 ± 0.7 | 5.8 ± 0.5 | 82.0 ± 4.0 |
| YDE-011 0.1 uM | | | |
| PBMC + LPS(5 ug/ml) + | 6.4 ± 0.3 | 5.0 ± 0.2 | 79.7 ± 5.8 |
| YDE-011 1 uM | | | |
| PBMC + LPS(5 ug/ml) + | 6.0 ± 0.4 | 5.3 ± 0.3 | 88.3 ± 1.5 |
| YDE-011 10 uM | | | |
| PBMC + LPS(5 ug/ml) + | 8.2 ± 0.4 | 6.1 ± 0.0 | 74.3 ± 4.1 |
| YDE-011 100 uM | | | |
| PBMC + LPS(5 ug/ml) + | 6.3 ± 0.7 | 5.0 ± 0.5 | 79.3 ± 1.5 |
| YDE-043 0.001 uM | | | |
| PBMC + LPS(5 ug/ml) + | 6.9 ± 0.4 | 5.4 ± 0.6 | 77.3 ± 6.9 |
| YDE-043 0.01 uM | | | |
| PBMC + LPS(5 ug/ml) + | 6.6 ± 0.1 | 5.4 ± 0.2 | 82.0 ± 4.0 |
| YDE-043 0.1 uM | | | |
| PBMC + LPS(5 ug/ml) + | 6.8 ± 0.6 | 5.3 ± 0.3 | 78.0 ± 5.0 |
| YDE-043 1 uM | | | |
| PBMC + LPS(5 ug/ml) + | 6.4 ± 0.1 | 5.4 ± 0.1 | 83.3 ± 3.3 |
| YDE-043 10 uM | | | |
| PBMC + LPS(5 ug/ml) + | 7.6 ± 0.6 | 6.3 ± 0.1 | 84.5 ± 4.5 |
| YDE-043 100 uM | | | |

### Statistical Analysis

All values are presented as mean±standard error of mean (SEM). The statistical significance of the results was analyzed using one-way ANOVA with a Bonferroni post hoc. The statistical analyses were performed using the SPSS software (SPSS 22.0, USA). Each compound treated PBMCs was compared to that of the stimulant induced PBMCs. The significant threshold was fixed at 0.05 i.e. p value has to be lower than 0.05 to be significant.

### Conclusions

LPS effectively induced various pro-inflammatory cytokines in human PBMCs and its induction was potently and significantly reduced by all compounds as low as 1 nM; whereas, the reference compound, Xiidra® was not as effective as the compounds. This finding is consistent with Example 8, suggesting that the compounds, especially YDE-011 and YDE043, are more potent and effective immunomodulators in lowering pro-inflammatory cytokine/chemokine productions in human PBMCs when stimulated by an endotoxin, LPS.

TABLE 25
A list of cytokines/chemokines and QC results

| | | Dilution | |
|---|---|---|---|
| No. | Cytokine | factor | Result |
| 1 | IL-10 | 1:10 | Within standard curve range |
| 2 | IL-8 | 1:200 | Within standard curve range |
| 3 | IL-6 | 1:200 | Within standard curve range |
| 4 | TNF-α | 1:10 | Within standard curve range |
| 5 | CCL3/MIP-1 α | 1:10 | Within standard curve range |
| 6 | CCL4/MIP-1 β | 1:10 | Within standard curve range |
| 7 | CCL5/RANTES | 1:2 | Within standard curve range |
| 8 | IFN-γ | 1:2 | Within standard curve range |
| 9 | CCL2/JE/MCP-1 | 1:2 | Within standard curve range |
| 10 | Fas Ligand | 1:2 | Within standard curve range |
| 11 | CCL20/MIP-3 α | 1:2 | Within standard curve range |
| 12 | IL-1 α | 1:2 | Within standard curve range |
| 13 | IL-1 β/IL-1F2 | 1:10 | Within standard curve range |
| 14 | TIMP-1 | 1:2 | Within standard curve range |
| 15 | GM-CSF | 1:2 | Within standard curve range |
| 16 | IL-3 | 1:2 | Below standard curve range |
| 17 | IL-17A | 1:2 | Below standard curve range |
| 18 | IL-4 | 1:2 | Below standard curve range |
| 19 | VEGF-A | 1:2 | Below standard curve range |
| 20 | MMP-9 | 1:2 | Below standard curve range |
| 21 | MMP-3 | 1:2 | BLQ |
| 22 | MMP-8 | 1:2 | BLQ |
| 23 | L-Selectin | 1:2 | BLQ |
| 24 | RAGE | 1:2 | BLQ |
| 25 | Fractalkine | 1:2 | BLQ |
| 26 | IL-5 | 1:2 | BLQ |
| 27 | IL-13 | 1:2 | BLQ |
| 28 | Leptin | 1:2 | BLQ |
| 29 | ICAM-1 | 1:2 | BLQ |
| 30 | VEGF-D | 1:2 | BLQ |

TABLE 26
Annex 1 - The count of PBMCs raw data
PBMCs count

| | | Total cell | | |
|---|---|---|---|---|
| | | count | Live cell | Viability |
| Sample | | (10\hat{ }5/ml) | (10\hat{ }5/ml) | (%) |
| PBMC + 0.5% DMSO | 1st | 8.7 | 7.5 | 86.0 |
| | 2nd | 8.5 | 7.4 | 87.0 |
| | 3rd | 7.5 | 6.6 | 89.0 |
| | Mean | 8.2 | 7.2 | 87.3 |
| | STDER | 0.4 | 0.3 | 0.9 |
| PBMC + LPS(5 ug/ml) | 1st | 7.3 | 6.1 | 84.0 |
| | 2nd | 6.7 | 5.8 | 86.0 |
| | 3rd | 6.4 | 5.5 | 85.0 |
| | Mean | 6.8 | 5.8 | 85.0 |
| | STDER | 0.3 | 0.2 | 0.6 |
| PBMC + YY101 0.01 uM | 1st | 6.3 | 5.2 | 83.0 |
| | 2nd | 5.5 | 4.8 | 87.0 |
| | Mean | 5.9 | 5.0 | 85.0 |
| | STDER | 0.4 | 0.2 | 2.0 |
| PBMC + YY101 0.1 uM | 1st | 5.2 | 4.4 | 84.0 |
| | 2nd | 6.9 | 5.8 | 84.0 |
| | Mean | 6.1 | 5.1 | 84.0 |
| | STDER | 0.8 | 0.7 | 0.0 |
| PBMC + YY101 1 uM | 1st | 5.9 | 5.5 | 92.0 |
| | 2nd | 6.3 | 5.5 | 87.0 |
| | Mean | 6.1 | 5.5 | 89.5 |
| | STDER | 0.2 | 0.0 | 2.5 |
| PBMC + YY101 10 uM | 1st | 7.6 | 6.4 | 85.0 |
| | 2nd | 7.1 | 6.1 | 87.0 |
| | Mean | 7.4 | 6.3 | 86.0 |
| | STDER | 0.3 | 0.2 | 1.0 |
| PBMC + YY101 100 uM | 1st | 7.3 | 6.4 | 88.0 |
| | 2nd | 8.2 | 7.0 | 85.0 |
| | Mean | 7.8 | 6.7 | 86.5 |
| | STDER | 0.5 | 0.3 | 1.5 |
| PBMC + LPS(5 ug/ml) + | 1st | 10.0 | 6.2 | 62.0 |
| Xiidra 0.001 uM | 2nd | 16.0 | 7.6 | 46.0 |
| | Mean | 13.0 | 6.9 | 54.0 |
| | STDER | 3.0 | 0.7 | 8.0 |
| PBMC + LPS(5 ug/ml) + | 1st | 6.3 | 5.2 | 82.0 |
| Xiidra 0.01 uM | 2nd | 7.4 | 5.8 | 78.0 |
| | Mean | 6.9 | 5.5 | 80.0 |
| | STDER | 0.6 | 0.3 | 2.0 |
| PBMC + LPS(5 ug/ml) + | 1st | 7.8 | 6.6 | 85.0 |
| Xiidra 0.1 uM | 2nd | 6.4 | 5.7 | 88.0 |
| | Mean | 7.1 | 6.2 | 86.5 |
| | STDER | 0.7 | 0.5 | 1.5 |
| PBMC + LPS(5 ug/ml) + | 1st | 6.0 | 4.8 | 81.0 |
| Xiidra 1 uM | 2nd | 5.5 | 4.4 | 80.0 |
| | Mean | 5.8 | 4.6 | 80.5 |
| | STDER | 0.3 | 0.2 | 0.5 |
| PBMC + LPS(5 ug/ml) + | 1st | 6.7 | 5.8 | 87.0 |
| Xiidra 10 uM | 2nd | 7.1 | 6.4 | 91.0 |
| | Mean | 6.9 | 6.1 | 89.0 |
| | STDER | 0.2 | 0.3 | 2.0 |
| PBMC + LPS(5 ug/ml) + | 1st | 8.3 | 7.1 | 85.0 |
| Xiidra 100 uM | 2nd | 6.6 | 5.8 | 88.0 |
| | Mean | 7.5 | 6.5 | 86.5 |
| | STDER | 0.9 | 0.7 | 1.5 |
| PBMC + LPS(5 ug/ml) + | 1st | 6.7 | 5.7 | 84.0 |
| YY101 0.001 uM | 2nd | 8.8 | 7.1 | 80.0 |
| | 3rd | 7.5 | 6.6 | 89.0 |
| | Mean | 7.7 | 6.5 | 84.3 |
| | STDER | 0.6 | 0.4 | 2.6 |
| PBMC + LPS(5 ug/ml) + | 1st | 9.2 | 6.4 | 70.0 |
| YY101 0.01 uM | 2nd | 8.1 | 6.0 | 75.0 |
| | 3rd | 7.0 | 6.1 | 87.0 |
| | Mean | 8.1 | 6.2 | 77.3 |
| | STDER | 0.6 | 0.1 | 5.0 |
| PBMC + LPS(5 ug/ml) + | 1st | 9.3 | 6.2 | 67.0 |
| YY101 0.1 uM | 2nd | 6.7 | 5.2 | 77.0 |
| | 3rd | 7.5 | 6.2 | 83.0 |
| | Mean | 7.8 | 5.9 | 75.7 |
| | STDER | 0.8 | 0.3 | 4.7 |
| PBMC + LPS(5 ug/ml) + | 1st | 8.8 | 6.3 | 72.0 |
| YY101 1 uM | 2nd | 9.7 | 7.1 | 73.0 |
| | 3rd | 11.0 | 6.2 | 55.0 |
| | Mean | 9.8 | 6.5 | 66.7 |
| | STDER | 0.6 | 0.3 | 5.8 |
| PBMC + LPS(5 ug/ml) + | 1st | 7.3 | 6.0 | 83.0 |
| YY101 10 uM | 2nd | 9.0 | 6.4 | 71.0 |
| | 3rd | 11.0 | 7.6 | 67.0 |
| | Mean | 9.1 | 6.7 | 73.7 |
| | STDER | 1.1 | 0.5 | 4.8 |
| PBMC + LPS(5 ug/ml) + | 1st | 8.7 | 6.3 | 73.0 |
| YY101 100 uM | 2nd | 8.8 | 6.2 | 70.0 |
| | 3rd | 5.8 | 5.0 | 87.0 |
| | Mean | 7.8 | 5.8 | 76.7 |
| | STDER | 1.0 | 0.4 | 5.2 |
| PBMC + LPS(5 ug/ml) + | 1st | 5.9 | 4.5 | 75.0 |
| YDE011 0.001 uM | 2nd | 6.3 | 5.8 | 92.0 |
| | 3rd | 6.6 | 5.8 | 88.0 |
| | Mean | 6.3 | 5.4 | 85.0 |
| | STDER | 0.2 | 0.4 | 5.1 |
| PBMC + LPS(5 ug/ml) + | 1st | 7.6 | 6.3 | 83.0 |
| YDE011 0.01 uM | 2nd | 7.8 | 6.2 | 81.0 |
| | 3rd | 6.4 | 5.1 | 79.0 |
| | Mean | 7.3 | 5.9 | 81.0 |
| | STDER | 0.4 | 0.4 | 1.2 |
| PBMC + LPS(5 ug/ml) + | 1st | 7.8 | 6.6 | 85.0 |
| YDE011 0.1 uM | 2nd | 5.7 | 5.0 | 87.0 |
| | 3rd | 7.7 | 5.7 | 74.0 |
| | Mean | 7.1 | 5.8 | 82.0 |
| | STDER | 0.7 | 0.5 | 4.0 |
| PBMC + LPS(5 ug/ml) + | 1st | 6.2 | 5.5 | 89.0 |
| YDE011 1 uM | 2nd | 7.0 | 4.8 | 69.0 |
| | 3rd | 5.9 | 4.8 | 81.0 |
| | Mean | 6.4 | 5.0 | 79.7 |
| | STDER | 0.3 | 0.2 | 5.8 |
| PBMC + LPS(5 ug/ml) + | 1st | 5.9 | 5.4 | 91.0 |
| YDE011 10 uM | 2nd | 6.7 | 5.8 | 86.0 |
| | 3rd | 5.4 | 4.7 | 88.0 |
| | Mean | 6.0 | 5.3 | 88.3 |
| | STDER | 0.4 | 0.3 | 1.5 |
| PBMC + LPS(5 ug/ml) + | 1st | 7.6 | 6.1 | 81.0 |
| YDE011 100 uM | 2nd | 8.1 | 6.1 | 75.0 |
| | 3rd | 9.0 | 6.0 | 67.0 |
| | Mean | 8.2 | 6.1 | 74.3 |
| | STDER | 0.4 | 0.0 | 4.1 |
| PBMC + LPS(5 ug/ml) + | 1st | 5.6 | 4.4 | 79.0 |
| YDE043 0.001 uM | 2nd | 7.8 | 6.0 | 77.0 |
| | 3rd | 5.6 | 4.6 | 82.0 |
| | Mean | 6.3 | 5.0 | 79.3 |
| | STDER | 0.7 | 0.5 | 1.5 |
| PBMC + LPS(5 ug/ml) + | 1st | 6.2 | 4.5 | 72.0 |
| YDE043 0.01 uM | 2nd | 7.3 | 6.6 | 91.0 |
| | 3rd | 7.2 | 5.0 | 69.0 |
| | Mean | 6.9 | 5.4 | 77.3 |
| | STDER | 0.4 | 0.6 | 6.9 |
| PBMC + LPS(5 ug/ml) + | 1st | 6.8 | 5.0 | 74.0 |
| YDE043 0.1 uM | 2nd | 6.4 | 5.6 | 87.0 |
| | 3rd | 6.5 | 5.6 | 85.0 |
| | Mean | 6.6 | 5.4 | 82.0 |
| | STDER | 0.1 | 0.2 | 4.0 |
| PBMC + LPS(5 ug/ml) + | 1st | 6.9 | 5.8 | 83.0 |
| YDE043 1 uM | 2nd | 5.7 | 4.8 | 83.0 |
| | 3rd | 7.8 | 5.3 | 68.0 |
| | Mean | 6.8 | 5.3 | 78.0 |
| | STDER | 0.6 | 0.3 | 5.0 |
| PBMC + LPS(5 ug/ml) + | 1st | 6.4 | 5.2 | 80.0 |
| YDE043 10 uM | 2nd | 6.7 | 5.4 | 80.0 |
| | 3rd | 6.2 | 5.6 | 90.0 |
| | Mean | 6.4 | 5.4 | 83.3 |
| | STDER | 0.1 | 0.1 | 3.3 |
| PBMC + LPS(5 ug/ml) + | 1st | 7.0 | 6.2 | 89.0 |
| YDE043 100 uM | 2nd | 8.1 | 6.4 | 80.0 |
| | Mean | 7.6 | 6.3 | 84.5 |
| | STDER | 0.6 | 0.1 | 4.5 |

TABLE 27
Annex 2-The main study raw data
Bead name : IL-10

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | −0.3 | 10.0 | NaN | | |
| | 2nd | 0.3 | 10.0 | NaN | | |
| | Mean | 0.0 | 10.0 | #DIV/0! | #DIV/0! | #DIV/0! |
| | STDER | 0.3 | 0.0 | #DIV/0! | #DIV/0! | #DIV/0! |
| PBMC + 0.5% DMSO | 1st | −0.3 | 10.0 | | 7.5 | 0.0 |
| | 2nd | −0.3 | 10.0 | | 7.4 | 0.0 |
| | 3rd | −0.3 | 10.0 | | 6.6 | 0.0 |
| | Mean | −0.3 | 10.0 | #DIV/0! | 7.2 | 0.0 |
| | STDER | 0.0 | 0.0 | #DIV/0! | 0.3 | 0.0 |
| PBMC + LPS(5 ug/ml) | 1st | 503.8 | 10.0 | 743.5 | 6.1 | 121.9 |
| | 2nd | 445.8 | 10.0 | 670.5 | 5.8 | 115.6 |
| | 3rd | 467.8 | 10.0 | 698.3 | 5.5 | 127.0 |
| | Mean | 472.4 | 10.0 | 704.1 | 5.8 | 121.5 |
| | STDER | 16.9 | 0.0 | 21.3 | 0.2 | 3.3 |
| PBMC + YY101 0.01 uM | 1st | 0.8 | 10.0 | | 5.2 | 0.0 |
| | 2nd | 1.8 | 10.0 | 4.3 | 4.8 | 0.9 |
| | Mean | 1.3 | 10.0 | 4.3 | 5.0 | 0.5 |
| | STDER | 0.5 | 0.0 | #DIV/0! | 0.2 | 0.5 |
| PBMC + YY101 0.1 uM | 1st | 0.8 | 10.0 | | 4.4 | 0.0 |
| | 2nd | 1.8 | 10.0 | 4.3 | 5.8 | 0.7 |
| | Mean | 1.3 | 10.0 | 4.3 | 5.1 | 0.4 |
| | STDER | 0.5 | 0.0 | #DIV/0! | 0.7 | 0.4 |
| PBMC + YY101 1 uM | 1st | 0.8 | 10.0 | | 5.5 | 0.0 |
| | 2nd | 0.8 | 10.0 | | 5.5 | 0.0 |
| | Mean | 0.8 | 10.0 | #DIV/0! | 5.5 | 0.0 |
| | STDER | 0.0 | 0.0 | #DIV/0! | 0.0 | 0.0 |
| PBMC + YY101 10 uM | 1st | 7.8 | 10.0 | 23.2 | 6.4 | 3.6 |
| | 2nd | 1.3 | 10.0 | 2.0 | 6.1 | 0.3 |
| | Mean | 4.5 | 10.0 | 12.6 | 6.3 | 2.0 |
| | STDER | 3.3 | 0.0 | 10.6 | 0.2 | 1.6 |
| PBMC + YY101 100 uM | 1st | 2.8 | 10.0 | 8.2 | 6.4 | 1.3 |
| | 2nd | −0.3 | 10.0 | | 7.0 | 0.0 |
| | Mean | 1.3 | 10.0 | 8.2 | 6.7 | 0.6 |
| | STDER | 1.5 | 0.0 | #DIV/0! | 0.3 | 0.6 |
| PBMC + LPS(5 ug/ml) + | 1st | 319.8 | 10.0 | 507.4 | 6.2 | 81.8 |
| Xiidra 0.001 uM | 2nd | 310.3 | 10.0 | 494.8 | 7.6 | 65.1 |
| | Mean | 315.0 | 10.0 | 501.1 | 6.9 | 73.5 |
| | STDER | 4.8 | 0.0 | 6.3 | 0.7 | 8.4 |
| PBMC + LPS(5 ug/ml) + | 1st | 298.8 | 10.0 | 479.5 | 5.2 | 92.2 |
| Xiidra 0.01 uM | 2nd | 327.3 | 10.0 | 517.4 | 5.8 | 89.2 |
| | Mean | 313.0 | 10.0 | 498.4 | 5.5 | 90.7 |
| | STDER | 14.3 | 0.0 | 18.9 | 0.3 | 1.5 |
| PBMC + LPS(5 ug/ml) + | 1st | 257.3 | 10.0 | 423.5 | 6.6 | 64.2 |
| Xiidra 0.1 uM | 2nd | 223.3 | 10.0 | 376.6 | 5.7 | 66.1 |
| | Mean | 240.3 | 10.0 | 400.0 | 6.2 | 65.1 |
| | STDER | 17.0 | 0.0 | 23.4 | 0.5 | 1.0 |
| PBMC + LPS(5 ug/ml) + | 1st | 305.8 | 10.0 | 488.8 | 4.8 | 101.8 |
| Xiidra 1 uM | 2nd | 219.3 | 10.0 | 371.0 | 4.4 | 84.3 |
| | Mean | 262.5 | 10.0 | 429.9 | 4.6 | 93.1 |
| | STDER | 43.3 | 0.0 | 58.9 | 0.2 | 8.8 |
| PBMC + LPS(5 ug/ml) + | 1st | 311.8 | 10.0 | 496.8 | 5.8 | 85.7 |
| Xiidra 10 uM | 2nd | 335.3 | 10.0 | 527.9 | 6.4 | 82.5 |
| | Mean | 323.5 | 10.0 | 512.4 | 6.1 | 84.1 |
| | STDER | 11.8 | 0.0 | 15.5 | 0.3 | 1.6 |
| PBMC + LPS(5 ug/ml) + | 1st | 329.8 | 10.0 | 520.7 | 7.1 | 73.3 |
| Xiidra 100 uM | 2nd | 318.8 | 10.0 | 506.1 | 5.8 | 87.3 |
| | Mean | 324.3 | 10.0 | 513.4 | 6.5 | 80.3 |
| | STDER | 5.5 | 0.0 | 7.3 | 0.7 | 7.0 |
| PBMC + LPS(5 ug/ml) + | 1st | 315.3 | 10.0 | 501.5 | 5.7 | 88.0 |
| YY101 0.001 uM | 2nd | 305.8 | 10.0 | 488.8 | 7.1 | 68.9 |
| | 3rd | 275.3 | 10.0 | 447.9 | 6.6 | 67.9 |
| | Mean | 298.8 | 10.0 | 479.4 | 6.5 | 74.9 |
| | STDER | 12.1 | 0.0 | 16.2 | 0.4 | 6.5 |
| PBMC + LPS(5 ug/ml) + | 1st | 308.8 | 10.0 | 492.8 | 6.4 | 77.0 |
| YY101 0.01 uM | 2nd | 313.8 | 10.0 | 499.5 | 6.0 | 83.2 |
| | 3rd | 319.8 | 10.0 | 507.4 | 6.1 | 83.2 |
| | Mean | 314.1 | 10.0 | 499.9 | 6.2 | 81.1 |
| | STDER | 3.2 | 0.0 | 4.2 | 0.1 | 2.1 |
| PBMC + LPS(5 ug/ml) + | 1st | 259.8 | 10.0 | 426.9 | 6.2 | 68.9 |
| YY101 0.1 uM | 2nd | 283.8 | 10.0 | 459.4 | 5.2 | 88.3 |
| | 3rd | 277.3 | 10.0 | 450.6 | 6.2 | 72.7 |
| | Mean | 273.6 | 10.0 | 445.6 | 5.9 | 76.6 |
| | STDER | 7.2 | 0.0 | 9.7 | 0.3 | 6.0 |
| PBMC + LPS(5 ug/ml) + | 1st | 357.3 | 10.0 | 556.7 | 6.3 | 88.4 |
| YY101 1 uM | 2nd | 302.8 | 10.0 | 484.8 | 7.1 | 68.3 |
| | 3rd | 327.8 | 10.0 | 518.0 | 6.2 | 83.6 |
| | Mean | 329.3 | 10.0 | 519.9 | 6.5 | 80.1 |
| | STDER | 15.8 | 0.0 | 20.8 | 0.3 | 6.1 |
| PBMC + LPS(5 ug/ml) + | 1st | 287.8 | 10.0 | 464.8 | 6.0 | 77.5 |
| YY101 10 uM | 2nd | 342.3 | 10.0 | 537.1 | 6.4 | 83.9 |
| | 3rd | 255.8 | 10.0 | 421.4 | 7.6 | 55.4 |
| | Mean | 295.3 | 10.0 | 474.4 | 6.7 | 72.3 |
| | STDER | 25.3 | 0.0 | 33.7 | 0.5 | 8.6 |
| PBMC + LPS(5 ug/ml) + | 1st | 86.8 | 10.0 | 173.5 | 6.3 | 27.5 |
| YY101 100 uM | 2nd | 83.8 | 10.0 | 168.6 | 6.2 | 27.2 |
| | 3rd | 87.8 | 10.0 | 175.1 | 5.0 | 35.0 |
| | Mean | 86.1 | 10.0 | 172.4 | 5.8 | 29.9 |
| | STDER | 1.2 | 0.0 | 2.0 | 0.4 | 2.6 |
| PBMC + LPS(5 ug/ml) + | 1st | 163.3 | 10.0 | 291.0 | 4.5 | 64.7 |
| YDE011 0.001 uM | 2nd | 179.3 | 10.0 | 314.2 | 5.8 | 54.2 |
| | 3rd | 173.8 | 10.0 | 306.3 | 5.8 | 52.8 |
| | Mean | 172.1 | 10.0 | 303.8 | 5.4 | 57.2 |
| | STDER | 4.7 | 0.0 | 6.8 | 0.4 | 3.7 |
| PBMC + LPS(5 ug/ml) + | 1st | 192.8 | 10.0 | 333.6 | 6.3 | 52.9 |
| YDE011 0.01 uM | 2nd | 186.8 | 10.0 | 325.0 | 6.2 | 52.4 |
| | 3rd | 166.3 | 10.0 | 295.4 | 5.1 | 57.9 |
| | Mean | 181.9 | 10.0 | 318.0 | 5.9 | 54.4 |
| | STDER | 8.0 | 0.0 | 11.6 | 0.4 | 1.7 |
| PBMC + LPS(5 ug/ml) + | 1st | 168.8 | 10.0 | 299.0 | 6.6 | 45.3 |
| YDE011 0.1 uM | 2nd | 181.8 | 10.0 | 317.8 | 5.0 | 63.6 |
| | 3rd | 176.3 | 10.0 | 309.9 | 5.7 | 54.4 |
| | Mean | 175.6 | 10.0 | 308.9 | 5.8 | 54.4 |
| | STDER | 3.8 | 0.0 | 5.5 | 0.5 | 5.3 |
| PBMC + LPS(5 ug/ml) + | 1st | 179.3 | 10.0 | 314.2 | 5.5 | 57.1 |
| YDE011 1 uM | 2nd | 182.8 | 10.0 | 319.3 | 4.8 | 66.5 |
| | 3rd | 176.8 | 10.0 | 310.6 | 4.8 | 64.7 |
| | Mean | 179.6 | 10.0 | 314.7 | 5.0 | 62.8 |
| | STDER | 1.7 | 0.0 | 2.5 | 0.2 | 2.9 |
| PBMC + LPS(5 ug/ml) + | 1st | 168.8 | 10.0 | 299.0 | 5.4 | 55.4 |
| YDE011 10 uM | 2nd | 166.3 | 10.0 | 295.4 | 5.8 | 50.9 |
| | 3rd | 174.8 | 10.0 | 307.7 | 4.7 | 65.5 |
| | Mean | 169.9 | 10.0 | 300.7 | 5.3 | 57.3 |
| | STDER | 2.5 | 0.0 | 3.7 | 0.3 | 4.3 |
| PBMC + LPS(5 ug/ml) + | 1st | 43.8 | 10.0 | 99.4 | 6.1 | 16.3 |
| YDE011 100 uM | 2nd | 51.8 | 10.0 | 113.9 | 6.1 | 18.7 |
| | 3rd | 55.8 | 10.0 | 121.1 | 6.0 | 20.2 |
| | Mean | 50.4 | 10.0 | 111.4 | 6.1 | 18.4 |
| | STDER | 3.5 | 0.0 | 6.4 | 0.0 | 1.1 |
| PBMC + LPS(5 ug/ml) + YDE043 0.001 uM | 1st | 194.3 | 10.0 | 335.7 | 4.4 | 76.3 |
| | 2nd | 168.3 | 10.0 | 298.3 | 6.0 | 49.7 |
| | 3rd | 157.8 | 10.0 | 282.9 | 4.6 | 61.5 |
| | Mean | 173.4 | 10.0 | 305.6 | 5.0 | 62.5 |
| | STDER | 10.8 | 0.0 | 15.7 | 0.5 | 7.7 |
| PBMC + LPS(5 ug/ml) + YDE043 0.01 uM | 1st | 200.8 | 10.0 | 344.9 | 4.5 | 76.7 |
| | 2nd | 213.3 | 10.0 | 362.6 | 6.6 | 54.9 |
| | 3rd | 188.3 | 10.0 | 327.1 | 5.0 | 65.4 |
| | Mean | 200.8 | 10.0 | 344.9 | 5.4 | 65.7 |
| | STDER | 7.2 | 0.0 | 10.2 | 0.6 | 6.3 |
| PBMC + LPS(5 ug/ml) + YDE043 0.1 uM | 1st | 190.3 | 10.0 | 330.0 | 5.0 | 66.0 |
| | 2nd | 178.3 | 10.0 | 312.8 | 5.6 | 55.9 |
| | 3rd | 186.3 | 10.0 | 324.3 | 5.6 | 57.9 |
| | Mean | 184.9 | 10.0 | 322.4 | 5.4 | 59.9 |
| | STDER | 3.5 | 0.0 | 5.1 | 0.2 | 3.1 |
| PBMC + LPS(5 ug/ml) + YDE043 1 uM | 1st | 189.3 | 10.0 | 328.6 | 5.8 | 56.7 |
| | 2nd | 178.3 | 10.0 | 312.8 | 4.8 | 65.2 |
| | 3rd | 198.3 | 10.0 | 341.4 | 5.3 | 64.4 |
| | Mean | 188.6 | 10.0 | 327.6 | 5.3 | 62.1 |
| | STDER | 5.8 | 0.0 | 8.3 | 0.3 | 2.7 |
| PBMC + LPS(5 ug/ml) + YDE043 10 uM | 1st | 182.3 | 10.0 | 318.5 | 5.2 | 61.3 |
| | 2nd | 153.8 | 10.0 | 277.0 | 5.4 | 51.3 |
| | 3rd | 167.8 | 10.0 | 297.5 | 5.6 | 53.1 |
| | Mean | 167.9 | 10.0 | 297.7 | 5.4 | 55.2 |
| | STDER | 8.2 | 0.0 | 12.0 | 0.1 | 3.1 |
| PBMC + LPS(5 ug/ml) + YDE043 100 uM | 1st | 47.8 | 10.0 | 106.7 | 6.2 | 17.2 |
| | 2nd | 50.8 | 10.0 | 112.1 | 6.4 | 17.5 |
| | Mean | 49.3 | 10.0 | 109.4 | 6.3 | 17.4 |
| | STDER | 1.5 | 0.0 | 2.7 | 0.1 | 0.2 |

TABLE 28
Bead name : IL-8

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | −0.5 | 200.0 | NaN | | |
| | 2nd | 0.5 | 200.0 | NaN | | |
| | Mean | 0.0 | 200.0 | #DIV/0! | #DIV/0! | #DIV/0! |
| | STDER | 0.5 | 0.0 | #DIV/0! | #DIV/0! | #DIV/0! |
| PBMC + 0.5% DMSO | 1st | 68.0 | 200.0 | 2052.8 | 7.5 | 273.7 |
| | 2nd | 70.5 | 200.0 | 2126.4 | 7.4 | 287.4 |
| | 3rd | 89.0 | 200.0 | 2671.9 | 6.6 | 404.8 |
| | Mean | 75.8 | 200.0 | 2283.7 | 7.2 | 322.0 |
| | STDER | 6.6 | 0.0 | 195.3 | 0.3 | 41.6 |
| PBMC + LPS(5 ug/ml) | 1st | 1590.5 | 200.0 | 48853.0 | 6.1 | 8008.7 |
| | 2nd | 1546.5 | 200.0 | 47437.7 | 5.8 | 8178.9 |
| | 3rd | 1575.5 | 200.0 | 48370.0 | 5.5 | 8794.5 |
| | Mean | 1570.8 | 200.0 | 48220.2 | 5.8 | 8327.4 |
| | STDER | 12.9 | 0.0 | 415.4 | 0.2 | 238.7 |
| PBMC + YY101 0.01 uM | 1st | 95.5 | 200.0 | 2863.8 | 5.2 | 550.7 |
| | 2nd | 230.0 | 200.0 | 6854.4 | 4.8 | 1428.0 |
| | Mean | 162.8 | 200.0 | 4859.1 | 5.0 | 989.4 |
| | STDER | 67.3 | 0.0 | 1995.3 | 0.2 | 438.6 |
| PBMC + YY101 0.1 uM | 1st | 90.0 | 200.0 | 2701.4 | 4.4 | 614.0 |
| | 2nd | 205.5 | 200.0 | 6125.1 | 5.8 | 1056.0 |
| | Mean | 147.8 | 200.0 | 4413.2 | 5.1 | 835.0 |
| | STDER | 57.8 | 0.0 | 1711.8 | 0.7 | 221.0 |
| PBMC + YY101 1 uM | 1st | 149.5 | 200.0 | 4461.8 | 5.5 | 811.2 |
| | 2nd | 157.5 | 200.0 | 4699.1 | 5.5 | 854.4 |
| | Mean | 153.5 | 200.0 | 4580.5 | 5.5 | 832.8 |
| | STDER | 4.0 | 0.0 | 118.6 | 0.0 | 21.6 |
| PBMC + YY101 10 uM | 1st | 1227.5 | 200.0 | 37311.2 | 6.4 | 5829.9 |
| | 2nd | 166.5 | 200.0 | 4966.2 | 6.1 | 814.1 |
| | Mean | 697.0 | 200.0 | 21138.7 | 6.3 | 3322.0 |
| | STDER | 530.5 | 0.0 | 16172.5 | 0.2 | 2507.9 |
| PBMC + YY101 100 uM | 1st | 664.5 | 200.0 | 19929.7 | 6.4 | 3114.0 |
| | 2nd | 358.5 | 200.0 | 10693.9 | 7.0 | 1527.7 |
| | Mean | 511.5 | 200.0 | 15311.8 | 6.7 | 2320.9 |
| | STDER | 153.0 | 0.0 | 4617.9 | 0.3 | 793.2 |
| PBMC + LPS(5 ug/ml) + | 1st | 1631.5 | 200.0 | 50176.1 | 6.2 | 8092.9 |
| Xiidra 0.001 uM | 2nd | 1424.5 | 200.0 | 43537.7 | 7.6 | 5728.6 |
| | Mean | 1528.0 | 200.0 | 46856.9 | 6.9 | 6910.8 |
| | STDER | 103.5 | 0.0 | 3319.2 | 0.7 | 1182.1 |
| PBMC + LPS(5 ug/ml) + | 1st | 1533.0 | 200.0 | 47004.4 | 5.2 | 9039.3 |
| Xiidra 0.01 uM | 2nd | 1638.0 | 200.0 | 50386.3 | 5.8 | 8687.3 |
| | Mean | 1585.5 | 200.0 | 48695.3 | 5.5 | 8863.3 |
| | STDER | 52.5 | 0.0 | 1691.0 | 0.3 | 176.0 |
| PBMC + LPS(5 ug/ml) + | 1st | 1396.5 | 200.0 | 42647.5 | 6.6 | 6461.7 |
| Xiidra 0.1 uM | 2nd | 1433.5 | 200.0 | 43824.2 | 5.7 | 7688.5 |
| | Mean | 1415.0 | 200.0 | 43235.9 | 6.2 | 7075.1 |
| | STDER | 18.5 | 0.0 | 588.4 | 0.5 | 613.4 |
| PBMC + LPS(5 ug/ml) + | 1st | 1382.5 | 200.0 | 42203.1 | 4.8 | 8792.3 |
| Xiidra 1 uM | 2nd | 1548.0 | 200.0 | 47485.8 | 4.4 | 10792.2 |
| | Mean | 1465.3 | 200.0 | 44844.5 | 4.6 | 9792.3 |
| | STDER | 82.8 | 0.0 | 2641.4 | 0.2 | 1000.0 |
| PBMC + LPS(5 ug/ml) + | 1st | 1418.5 | 200.0 | 43346.8 | 5.8 | 7473.6 |
| Xiidra 10 uM | 2nd | 1370.5 | 200.0 | 41822.5 | 6.4 | 6534.8 |
| | Mean | 1394.5 | 200.0 | 42584.6 | 6.1 | 7004.2 |
| | STDER | 24.0 | 0.0 | 762.2 | 0.3 | 469.4 |
| PBMC + LPS(5 ug/ml) + | 1st | 1356.0 | 200.0 | 41363.0 | 7.1 | 5825.8 |
| Xiidra 100 uM | 2nd | 1346.5 | 200.0 | 41062.3 | 5.8 | 7079.7 |
| | Mean | 1351.3 | 200.0 | 41212.6 | 6.5 | 6452.7 |
| | STDER | 4.8 | 0.0 | 150.4 | 0.7 | 627.0 |
| PBMC + LPS(5 ug/ml) + | 1st | 1479.5 | 200.0 | 45291.5 | 5.7 | 7945.9 |
| YY101 0.001 uM | 2nd | 1465.5 | 200.0 | 44844.4 | 7.1 | 6316.1 |
| | 3rd | 1677.0 | 200.0 | 51649.5 | 6.6 | 7825.7 |
| | Mean | 1540.7 | 200.0 | 47261.8 | 6.5 | 7362.6 |
| | STDER | 68.3 | 0.0 | 2197.6 | 0.4 | 524.4 |
| PBMC + LPS(5 ug/ml) + | 1st | 1486.0 | 200.0 | 45499.3 | 6.4 | 7109.3 |
| YY101 0.01 uM | 2nd | 1465.5 | 200.0 | 44844.4 | 6.0 | 7474.1 |
| | 3rd | 1515.5 | 200.0 | 46443.3 | 6.1 | 7613.7 |
| | Mean | 1489.0 | 200.0 | 45595.7 | 6.2 | 7399.0 |
| | STDER | 14.5 | 0.0 | 464.1 | 0.1 | 150.4 |
| PBMC + LPS(5 ug/ml) + | 1st | 1353.5 | 200.0 | 41283.9 | 6.2 | 6658.7 |
| YY101 0.1 uM | 2nd | 1407.5 | 200.0 | 42997.0 | 5.2 | 8268.7 |
| | 3rd | 1359.5 | 200.0 | 41473.9 | 6.2 | 6689.3 |
| | Mean | 1373.5 | 200.0 | 41918.3 | 5.9 | 7205.6 |
| | STDER | 17.1 | 0.0 | 542.2 | 0.3 | 531.6 |
| PBMC + LPS(5 ug/ml) + | 1st | 1476.5 | 200.0 | 45195.7 | 6.3 | 7173.9 |
| YY101 1 uM | 2nd | 1478.5 | 200.0 | 45259.6 | 7.1 | 6374.6 |
| | 3rd | 1560.0 | 200.0 | 47871.4 | 6.2 | 7721.2 |
| | Mean | 1505.0 | 200.0 | 46108.9 | 6.5 | 7089.9 |
| | STDER | 27.5 | 0.0 | 881.4 | 0.3 | 391.0 |
| PBMC + LPS(5 ug/ml) + | 1st | 1511.5 | 200.0 | 46315.2 | 6.0 | 7719.2 |
| YY101 10 uM | 2nd | 1649.0 | 200.0 | 50742.2 | 6.4 | 7928.5 |
| | 3rd | 1407.0 | 200.0 | 42981.1 | 7.6 | 5655.4 |
| | Mean | 1522.5 | 200.0 | 46679.5 | 6.7 | 7101.0 |
| | STDER | 70.1 | 0.0 | 2247.8 | 0.5 | 725.3 |
| PBMC + LPS(5 ug/ml) + | 1st | 1574.5 | 200.0 | 48337.8 | 6.3 | 7672.7 |
| YY101 100 uM | 2nd | 1532.5 | 200.0 | 46988.3 | 6.2 | 7578.8 |
| | 3rd | 1444.5 | 200.0 | 44174.7 | 5.0 | 8834.9 |
| | Mean | 1517.2 | 200.0 | 46500.3 | 5.8 | 8028.8 |
| | STDER | 38.3 | 0.0 | 1226.3 | 0.4 | 404.0 |
| PBMC + LPS(5 ug/ml) + | 1st | 861.5 | 200.0 | 25949.1 | 4.5 | 5766.5 |
| YDE011 0.001 uM | 2nd | 838.5 | 200.0 | 25243.2 | 5.8 | 4352.3 |
| | 3rd | 906.5 | 200.0 | 27333.0 | 5.8 | 4712.6 |
| | Mean | 868.8 | 200.0 | 26175.1 | 5.4 | 4943.8 |
| | STDER | 20.0 | 0.0 | 613.8 | 0.4 | 424.3 |
| PBMC + LPS(5 ug/ml) + | 1st | 906.0 | 200.0 | 27317.6 | 6.3 | 4336.1 |
| YDE011 0.01 uM | 2nd | 964.0 | 200.0 | 29106.4 | 6.2 | 4694.6 |
| | 3rd | 900.5 | 200.0 | 27148.3 | 5.1 | 5323.2 |
| | Mean | 923.5 | 200.0 | 27857.4 | 5.9 | 4784.6 |
| | STDER | 20.3 | 0.0 | 626.4 | 0.4 | 288.5 |
| PBMC + LPS(5 ug/ml) + | 1st | 852.5 | 200.0 | 25672.8 | 6.6 | 3889.8 |
| YDE011 0.1 uM | 2nd | 870.5 | 200.0 | 26225.6 | 5.0 | 5245.1 |
| | 3rd | 853.5 | 200.0 | 25703.5 | 5.7 | 4509.4 |
| | Mean | 858.8 | 200.0 | 25867.3 | 5.8 | 4548.1 |
| | STDER | 5.8 | 0.0 | 179.4 | 0.5 | 391.7 |
| PBMC + LPS(5 ug/ml) + | 1st | 872.0 | 200.0 | 26271.7 | 5.5 | 4776.7 |
| YDE011 1 uM | 2nd | 875.5 | 200.0 | 26379.3 | 4.8 | 5495.7 |
| | 3rd | 805.5 | 200.0 | 24231.7 | 4.8 | 5048.3 |
| | Mean | 851.0 | 200.0 | 25627.6 | 5.0 | 5106.9 |
| | STDER | 22.8 | 0.0 | 698.6 | 0.2 | 209.6 |
| PBMC + LPS(5 ug/ml) + | 1st | 851.0 | 200.0 | 25626.7 | 5.4 | 4745.7 |
| YDE011 10 uM | 2nd | 841.5 | 200.0 | 25335.2 | 5.8 | 4368.1 |
| | 3rd | 809.5 | 200.0 | 24354.2 | 4.7 | 5181.8 |
| | Mean | 834.0 | 200.0 | 25105.4 | 5.3 | 4765.2 |
| | STDER | 12.6 | 0.0 | 384.9 | 0.3 | 235.1 |
| PBMC + LPS(5 ug/ml) + | 1st | 918.5 | 200.0 | 27702.6 | 6.1 | 4541.4 |
| YDE011 100 uM | 2nd | 881.5 | 200.0 | 26563.8 | 6.1 | 4354.7 |
| | 3rd | 793.0 | 200.0 | 23849.1 | 6.0 | 3974.8 |
| | Mean | 864.3 | 200.0 | 26038.5 | 6.1 | 4290.3 |
| | STDER | 37.2 | 0.0 | 1143.0 | 0.0 | 166.7 |
| PBMC + LPS(5 ug/ml) + | 1st | 841.0 | 200.0 | 25319.8 | 4.4 | 5754.5 |
| YDE043 0.001 uM | 2nd | 932.5 | 200.0 | 28134.2 | 6.0 | 4689.0 |
| | 3rd | 973.0 | 200.0 | 29384.5 | 4.6 | 6387.9 |
| | Mean | 915.5 | 200.0 | 27612.8 | 5.0 | 5610.5 |
| | STDER | 39.0 | 0.0 | 1202.0 | 0.5 | 495.7 |
| PBMC + LPS(5 ug/ml) + | 1st | 928.5 | 200.0 | 28010.8 | 4.5 | 6224.6 |
| YDE043 0.01 uM | 2nd | 897.5 | 200.0 | 27056.0 | 6.6 | 4099.4 |
| | 3rd | 939.5 | 200.0 | 28350.1 | 5.0 | 5670.0 |
| | Mean | 921.8 | 200.0 | 27805.6 | 5.4 | 5331.3 |
| | STDER | 12.6 | 0.0 | 387.4 | 0.6 | 636.4 |
| PBMC + LPS(5 ug/ml) + | 1st | 912.0 | 200.0 | 27502.4 | 5.0 | 5500.5 |
| YDE043 0.1 uM | 2nd | 822.0 | 200.0 | 24737.2 | 5.6 | 4417.4 |
| | 3rd | 926.5 | 200.0 | 27949.2 | 5.6 | 4990.9 |
| | Mean | 886.8 | 200.0 | 26729.6 | 5.4 | 4969.6 |
| | STDER | 32.7 | 0.0 | 1004.5 | 0.2 | 312.9 |
| PBMC + LPS(5 ug/ml) + | 1st | 912.5 | 200.0 | 27517.8 | 5.8 | 4744.4 |
| YDE043 1 uM | 2nd | 765.5 | 200.0 | 23008.1 | 4.8 | 4793.4 |
| | 3rd | 954.0 | 200.0 | 28797.5 | 5.3 | 5433.5 |
| | Mean | 877.3 | 200.0 | 26441.2 | 5.3 | 4990.4 |
| | STDER | 57.2 | 0.0 | 1755.8 | 0.3 | 222.0 |
| PBMC + LPS(5 ug/ml) + | 1st | 960.5 | 200.0 | 28998.3 | 5.2 | 5576.6 |
| YDE043 10 uM | 2nd | 1141.5 | 200.0 | 34618.6 | 5.4 | 6410.8 |
| | 3rd | 812.5 | 200.0 | 24446.1 | 5.6 | 4365.4 |
| | Mean | 971.5 | 200.0 | 29354.3 | 5.4 | 5450.9 |
| | STDER | 95.1 | 0.0 | 2941.9 | 0.1 | 593.8 |
| PBMC + LPS(5 ug/ml) + | 1st | 895.5 | 200.0 | 26994.4 | 6.2 | 4353.9 |
| YDE043 100 uM | 2nd | 798.5 | 200.0 | 24017.4 | 6.4 | 3752.7 |
| | Mean | 847.0 | 200.0 | 25505.9 | 6.3 | 4053.3 |
| | STDER | 48.5 | 0.0 | 1488.5 | 0.1 | 300.6 |

TABLE 29
Bead name: IL-6

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.3 | 200.0 | NaN | | |
| | 2nd | −0.3 | 200.0 | NaN | | |
| | Mean | 0.0 | 200.0 | #DIV/0! | #DIV/0! | #DIV/0! |
| | STDER | 0.3 | 0.0 | #DIV/0! | #DIV/0! | #DIV/0! |
| PBMC + 0.5% DMSO | 1st | 2.3 | 200.0 | | 7.5 | 0.0 |
| | 2nd | 3.8 | 200.0 | | 7.4 | 0.0 |
| | 3rd | 2.3 | 200.0 | | 6.6 | 0.0 |
| | Mean | 2.8 | 200.0 | #DIV/0! | 7.2 | 0.0 |
| | STDER | 0.5 | 0.0 | #DIV/0! | 0.3 | 0.0 |
| PBMC + LPS(5 ug/ml) | 1st | 431.3 | 200.0 | 15083.7 | 6.1 | 2472.7 |
| | 2nd | 423.3 | 200.0 | 14810.7 | 5.8 | 2553.6 |
| | 3rd | 407.3 | 200.0 | 14264.9 | 5.5 | 2593.6 |
| | Mean | 420.6 | 200.0 | 14719.8 | 5.8 | 2540.0 |
| | STDER | 7.1 | 0.0 | 240.7 | 0.2 | 35.5 |
| PBMC + YY101 0.01 uM | 1st | 4.3 | 200.0 | | 5.2 | 0.0 |
| | 2nd | 2.3 | 200.0 | | 4.8 | 0.0 |
| | Mean | 3.3 | 200.0 | #DIV/0! | 5.0 | 0.0 |
| | STDER | 1.0 | 0.0 | #DIV/0! | 0.2 | 0.0 |
| PBMC + YY101 0.1 uM | 1st | 1.3 | 200.0 | | 4.4 | 0.0 |
| | 2nd | 1.3 | 200.0 | | 5.8 | 0.0 |
| | Mean | 1.3 | 200.0 | #DIV/0! | 5.1 | 0.0 |
| | STDER | 0.0 | 0.0 | #DIV/0! | 0.7 | 0.0 |
| PBMC + YY101 1 uM | 1st | 2.3 | 200.0 | | 5.5 | 0.0 |
| | 2nd | 1.3 | 200.0 | | 5.5 | 0.0 |
| | Mean | 1.8 | 200.0 | #DIV/0! | 5.5 | 0.0 |
| | STDER | 0.5 | 0.0 | #DIV/0! | 0.0 | 0.0 |
| PBMC + YY101 10 uM | 1st | 27.3 | 200.0 | 880.7 | 6.4 | 137.6 |
| | 2nd | 3.3 | 200.0 | | 6.1 | 0.0 |
| | Mean | 15.3 | 200.0 | 880.7 | 6.3 | 68.8 |
| | STDER | 12.0 | 0.0 | #DIV/0! | 0.2 | 68.8 |
| PBMC + YY101 100 uM | 1st | 19.8 | 200.0 | 524.7 | 6.4 | 82.0 |
| | 2nd | 11.8 | 200.0 | | 7.0 | 0.0 |
| | Mean | 15.8 | 200.0 | 524.7 | 6.7 | 41.0 |
| | STDER | 4.0 | 0.0 | #DIV/0! | 0.3 | 41.0 |
| PBMC + LPS(5 ug/ml) + | 1st | 415.8 | 200.0 | 14554.8 | 6.2 | 2347.5 |
| Xiidra 0.001 uM | 2nd | 365.3 | 200.0 | 12833.4 | 7.6 | 1688.6 |
| | Mean | 390.5 | 200.0 | 13694.1 | 6.9 | 2018.1 |
| | STDER | 25.3 | 0.0 | 860.7 | 0.7 | 329.5 |
| PBMC + LPS(5 ug/ml) + | 1st | 427.3 | 200.0 | 14947.2 | 5.2 | 2874.5 |
| Xiidra 0.01 uM | 2nd | 380.3 | 200.0 | 13344.5 | 5.8 | 2300.8 |
| | Mean | 403.8 | 200.0 | 14145.8 | 5.5 | 2587.6 |
| | STDER | 23.5 | 0.0 | 801.4 | 0.3 | 286.8 |
| PBMC + LPS(5 ug/ml) + | 1st | 367.8 | 200.0 | 12918.6 | 6.6 | 1957.4 |
| Xiidra 0.1 uM | 2nd | 384.3 | 200.0 | 13480.8 | 5.7 | 2365.0 |
| | Mean | 376.0 | 200.0 | 13199.7 | 6.2 | 2161.2 |
| | STDER | 8.3 | 0.0 | 281.1 | 0.5 | 203.8 |
| PBMC + LPS(5 ug/ml) + | 1st | 405.8 | 200.0 | 14213.7 | 4.8 | 2961.2 |
| Xiidra 1 uM | 2nd | 428.8 | 200.0 | 14998.4 | 4.4 | 3408.7 |
| | Mean | 417.3 | 200.0 | 14606.0 | 4.6 | 3185.0 |
| | STDER | 11.5 | 0.0 | 392.3 | 0.2 | 223.8 |
| PBMC + LPS(5 ug/ml) + | 1st | 431.3 | 200.0 | 15083.7 | 5.8 | 2600.6 |
| Xiidra 10 uM | 2nd | 400.3 | 200.0 | 14026.2 | 6.4 | 2191.6 |
| | Mean | 415.8 | 200.0 | 14554.9 | 6.1 | 2396.1 |
| | STDER | 15.5 | 0.0 | 528.8 | 0.3 | 204.5 |
| PBMC + LPS(5 ug/ml) + | 1st | 407.8 | 200.0 | 14281.9 | 7.1 | 2011.5 |
| Xiidra 100 uM | 2nd | 426.3 | 200.0 | 14913.1 | 5.8 | 2571.2 |
| | Mean | 417.0 | 200.0 | 14597.5 | 6.5 | 2291.4 |
| | STDER | 9.3 | 0.0 | 315.6 | 0.7 | 279.8 |
| PBMC + LPS(5 ug/ml) + | 1st | 371.3 | 200.0 | 13037.8 | 5.7 | 2287.3 |
| YY101 0.001 uM | 2nd | 359.8 | 200.0 | 12646.1 | 7.1 | 1781.1 |
| | 3rd | 335.3 | 200.0 | 11811.7 | 6.6 | 1789.6 |
| | Mean | 355.4 | 200.0 | 12498.5 | 6.5 | 1952.7 |
| | STDER | 10.6 | 0.0 | 361.6 | 0.4 | 167.3 |
| PBMC + LPS(5 ug/ml) + | 1st | 378.3 | 200.0 | 13276.3 | 6.4 | 2074.4 |
| YY101 0.01 uM | 2nd | 385.8 | 200.0 | 13531.9 | 6.0 | 2255.3 |
| | 3rd | 373.3 | 200.0 | 13106.0 | 6.1 | 2148.5 |
| | Mean | 379.1 | 200.0 | 13304.7 | 6.2 | 2159.4 |
| | STDER | 3.6 | 0.0 | 123.8 | 0.1 | 52.5 |
| | 1st | 353.8 | 200.0 | 12441.7 | 6.2 | 2006.7 |
| PBMC + LPS(5 ug/ml) + | 2nd | 374.8 | 200.0 | 13157.1 | 5.2 | 2530.2 |
| YY101 0.1 uM | 3rd | 365.8 | 200.0 | 12850.5 | 6.2 | 2072.7 |
| | Mean | 364.8 | 200.0 | 12816.4 | 5.9 | 2203.2 |
| | STDER | 6.1 | 0.0 | 207.2 | 0.3 | 164.6 |
| PBMC + LPS(5 ug/ml) + | 1st | 377.3 | 200.0 | 13242.2 | 6.3 | 2101.9 |
| YY101 1 uM | 2nd | 366.3 | 200.0 | 12867.5 | 7.1 | 1812.3 |
| | 3rd | 368.8 | 200.0 | 12952.6 | 6.2 | 2089.1 |
| | Mean | 370.8 | 200.0 | 13020.8 | 6.5 | 2001.1 |
| | STDER | 3.3 | 0.0 | 113.4 | 0.3 | 94.5 |
| PBMC + LPS(5 ug/ml) + | 1st | 398.8 | 200.0 | 13975.0 | 6.0 | 2329.2 |
| YY101 10 uM | 2nd | 430.3 | 200.0 | 15049.6 | 6.4 | 2351.5 |
| | 3rd | 371.3 | 200.0 | 13037.8 | 7.6 | 1715.5 |
| | Mean | 400.1 | 200.0 | 14020.8 | 6.7 | 2132.1 |
| | STDER | 17.0 | 0.0 | 581.2 | 0.5 | 208.4 |
| PBMC + LPS(5 ug/ml) + | 1st | 462.3 | 200.0 | 16142.7 | 6.3 | 2562.3 |
| YY101 100 uM | 2nd | 442.3 | 200.0 | 15459.3 | 6.2 | 2493.4 |
| | 3rd | 428.8 | 200.0 | 14998.4 | 5.0 | 2999.7 |
| | Mean | 444.4 | 200.0 | 15533.5 | 5.8 | 2685.2 |
| | STDER | 9.7 | 0.0 | 332.4 | 0.4 | 158.5 |
| PBMC + LPS(5 ug/ml) + | 1st | 179.8 | 200.0 | 6493.4 | 4.5 | 1443.0 |
| YDE011 0.001 uM | 2nd | 185.3 | 200.0 | 6683.4 | 5.8 | 1152.3 |
| | 3rd | 194.8 | 200.0 | 7011.0 | 5.8 | 1208.8 |
| | Mean | 186.6 | 200.0 | 6729.3 | 5.4 | 1268.0 |
| | STDER | 4.4 | 0.0 | 151.1 | 0.4 | 89.0 |
| PBMC + LPS(5 ug/ml) + | 1st | 201.3 | 200.0 | 7234.7 | 6.3 | 1148.4 |
| YDE011 0.01 uM | 2nd | 198.3 | 200.0 | 7131.5 | 6.2 | 1150.2 |
| | 3rd | 192.3 | 200.0 | 6924.8 | 5.1 | 1357.8 |
| | Mean | 197.3 | 200.0 | 7097.0 | 5.9 | 1218.8 |
| | STDER | 2.6 | 0.0 | 91.1 | 0.4 | 69.5 |
| PBMC + LPS(5 ug/ml) + | 1st | 178.8 | 200.0 | 6458.9 | 6.6 | 978.6 |
| YDE011 0.1 uM | 2nd | 191.8 | 200.0 | 6907.6 | 5.0 | 1381.5 |
| | 3rd | 186.8 | 200.0 | 6735.2 | 5.7 | 1181.6 |
| | Mean | 185.8 | 200.0 | 6700.6 | 5.8 | 1180.6 |
| | STDER | 3.8 | 0.0 | 130.7 | 0.5 | 116.3 |
| PBMC + LPS(5 ug/ml) + | 1st | 189.3 | 200.0 | 6821.4 | 5.5 | 1240.3 |
| YDE011 1 uM | 2nd | 200.8 | 200.0 | 7217.5 | 4.8 | 1503.6 |
| | 3rd | 179.3 | 200.0 | 6476.2 | 4.8 | 1349.2 |
| | Mean | 189.8 | 200.0 | 6838.4 | 5.0 | 1364.4 |
| | STDER | 6.2 | 0.0 | 214.2 | 0.2 | 76.4 |
| PBMC + LPS(5 ug/ml) + | 1st | 191.3 | 200.0 | 6890.4 | 5.4 | 1276.0 |
| YDE011 10 uM | 2nd | 185.8 | 200.0 | 6700.7 | 5.8 | 1155.3 |
| | 3rd | 186.3 | 200.0 | 6717.9 | 4.7 | 1429.3 |
| | Mean | 187.8 | 200.0 | 6769.7 | 5.3 | 1286.9 |
| | STDER | 1.8 | 0.0 | 60.6 | 0.3 | 79.3 |
| PBMC + LPS(5 ug/ml) + | 1st | 217.8 | 200.0 | 7801.4 | 6.1 | 1278.9 |
| YDE011 100 uM | 2nd | 215.3 | 200.0 | 7715.7 | 6.1 | 1264.9 |
| | 3rd | 200.3 | 200.0 | 7200.3 | 6.0 | 1200.1 |
| | Mean | 211.1 | 200.0 | 7572.5 | 6.1 | 1247.9 |
| | STDER | 5.5 | 0.0 | 187.7 | 0.0 | 24.3 |
| PBMC + LPS(5 ug/ml) + | 1st | 175.8 | 200.0 | 6355.1 | 4.4 | 1444.3 |
| YDE043 0.001 uM | 2nd | 211.3 | 200.0 | 7578.4 | 6.0 | 1263.1 |
| | 3rd | 204.3 | 200.0 | 7337.9 | 4.6 | 1595.2 |
| | Mean | 197.1 | 200.0 | 7090.4 | 5.0 | 1434.2 |
| | STDER | 10.9 | 0.0 | 374.2 | 0.5 | 96.0 |
| PBMC + LPS(5 ug/ml) + | 1st | 196.3 | 200.0 | 7062.6 | 4.5 | 1569.5 |
| YDE043 0.01 uM | 2nd | 194.8 | 200.0 | 7011.0 | 6.6 | 1062.3 |
| | 3rd | 187.3 | 200.0 | 6752.4 | 5.0 | 1350.5 |
| | Mean | 192.8 | 200.0 | 6942.0 | 5.4 | 1327.4 |
| | STDER | 2.8 | 0.0 | 96.0 | 0.6 | 146.9 |
| PBMC + LPS(5 ug/ml) + | 1st | 180.8 | 200.0 | 6528.0 | 5.0 | 1305.6 |
| YDE043 0.1 uM | 2nd | 169.8 | 200.0 | 6147.3 | 5.6 | 1097.7 |
| | 3rd | 197.3 | 200.0 | 7097.1 | 5.6 | 1267.3 |
| | Mean | 182.6 | 200.0 | 6590.8 | 5.4 | 1223.6 |
| | STDER | 8.0 | 0.0 | 276.0 | 0.2 | 63.9 |
| PBMC + LPS(5 ug/ml) + | 1st | 198.3 | 200.0 | 7131.5 | 5.8 | 1229.6 |
| YDE043 1 uM | 2nd | 170.3 | 200.0 | 6164.6 | 4.8 | 1284.3 |
| | 3rd | 214.3 | 200.0 | 7681.3 | 5.3 | 1449.3 |
| | Mean | 194.3 | 200.0 | 6992.5 | 5.3 | 1321.1 |
| | STDER | 12.9 | 0.0 | 443.3 | 0.3 | 66.0 |
| PBMC + LPS(5 ug/ml) + | 1st | 203.3 | 200.0 | 7303.5 | 5.2 | 1404.5 |
| YDE043 10 uM | 2nd | 415.8 | 200.0 | 14554.8 | 5.4 | 2695.3 |
| | 3rd | 185.3 | 200.0 | 6683.4 | 5.6 | 1193.5 |
| | Mean | 268.1 | 200.0 | 9513.9 | 5.4 | 1764.4 |
| | STDER | 74.0 | 0.0 | 2526.8 | 0.1 | 469.4 |
| PBMC + LPS(5 ug/ml) + | 1st | 205.8 | 200.0 | 7389.4 | 6.2 | 1191.8 |
| YDE043 100 uM | 2nd | 199.8 | 200.0 | 7183.1 | 6.4 | 1122.4 |
| | Mean | 202.8 | 200.0 | 7286.3 | 6.3 | 1157.1 |
| | STDER | 3.0 | 0.0 | 103.2 | 0.1 | 34.7 |

TABLE 30
Bead name: TNF-alpha

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.0 | 10.0 | NaN | | |
| | 2nd | 0.0 | 10.0 | NaN | | |
| | Mean | 0.0 | 10.0 | #DIV/0! | #DIV/0! | #DIV/0! |
| | STDER | 0.0 | 0.0 | #DIV/0! | #DIV/0! | #DIV/0! |
| PBMC + 0.5% DMSO | 1st | 1.0 | 10.0 | 16.4 | 7.5 | 2.2 |
| | 2nd | 2.0 | 10.0 | 19.0 | 7.4 | 2.6 |
| | 3rd | 1.5 | 10.0 | 17.7 | 6.6 | 2.7 |
| | Mean | 1.5 | 10.0 | 17.7 | 7.2 | 2.5 |
| | STDER | 0.3 | 0.0 | 0.7 | 0.3 | 0.1 |
| PBMC + LPS(5 ug/ml) | 1st | 1068.5 | 10.0 | 3196.1 | 6.1 | 524.0 |
| | 2nd | 986.0 | 10.0 | 2941.3 | 5.8 | 507.1 |
| | 3rd | 943.5 | 10.0 | 2810.4 | 5.5 | 511.0 |
| | Mean | 999.3 | 10.0 | 2982.6 | 5.8 | 514.0 |
| | STDER | 36.7 | 0.0 | 113.2 | 0.2 | 5.1 |
| PBMC + YY101 0.01 uM | 1st | 5.0 | 10.0 | 26.8 | 5.2 | 5.1 |
| | 2nd | 6.0 | 10.0 | 29.4 | 4.8 | 6.1 |
| | Mean | 5.5 | 10.0 | 28.1 | 5.0 | 5.6 |
| | STDER | 0.5 | 0.0 | 1.3 | 0.2 | 0.5 |
| PBMC + YY101 0.1 uM | 1st | 5.0 | 10.0 | 26.8 | 4.4 | 6.1 |
| | 2nd | 7.0 | 10.0 | 32.0 | 5.8 | 5.5 |
| | Mean | 6.0 | 10.0 | 29.4 | 5.1 | 5.8 |
| | STDER | 1.0 | 0.0 | 2.6 | 0.7 | 0.3 |
| PBMC + YY101 1 uM | 1st | 7.0 | 10.0 | 32.0 | 5.5 | 5.8 |
| | 2nd | 8.0 | 10.0 | 34.6 | 5.5 | 6.3 |
| | Mean | 7.5 | 10.0 | 33.3 | 5.5 | 6.1 |
| | STDER | 0.5 | 0.0 | 1.3 | 0.0 | 0.2 |
| PBMC + YY101 10 uM | 1st | 29.0 | 10.0 | 90.9 | 6.4 | 14.2 |
| | 2nd | 7.0 | 10.0 | 32.0 | 6.1 | 5.2 |
| | Mean | 18.0 | 10.0 | 61.4 | 6.3 | 9.7 |
| | STDER | 11.0 | 0.0 | 29.4 | 0.2 | 4.5 |
| PBMC + YY101 100 uM | 1st | 30.5 | 10.0 | 95.0 | 6.4 | 14.8 |
| | 2nd | 14.0 | 10.0 | 50.5 | 7.0 | 7.2 |
| | Mean | 22.3 | 10.0 | 72.7 | 6.7 | 11.0 |
| | STDER | 8.3 | 0.0 | 22.2 | 0.3 | 3.8 |
| PBMC + LPS(5 ug/ml) + | 1st | 861.0 | 10.0 | 2557.1 | 6.2 | 412.4 |
| Xiidra 0.001 uM | 2nd | 841.0 | 10.0 | 2495.8 | 7.6 | 328.4 |
| | Mean | 851.0 | 10.0 | 2526.4 | 6.9 | 370.4 |
| | STDER | 10.0 | 0.0 | 30.6 | 0.7 | 42.0 |
| PBMC + LPS(5 ug/ml) + | 1st | 1000.0 | 10.0 | 2984.4 | 5.2 | 573.9 |
| Xiidra 0.01 uM | 2nd | 837.0 | 10.0 | 2483.6 | 5.8 | 428.2 |
| | Mean | 918.5 | 10.0 | 2734.0 | 5.5 | 501.1 |
| | STDER | 81.5 | 0.0 | 250.4 | 0.3 | 72.9 |
| PBMC + LPS(5 ug/ml) + | 1st | 869.0 | 10.0 | 2581.6 | 6.6 | 391.1 |
| Xiidra 0.1 uM | 2nd | 751.0 | 10.0 | 2221.0 | 5.7 | 389.6 |
| | Mean | 810.0 | 10.0 | 2401.3 | 6.2 | 390.4 |
| | STDER | 59.0 | 0.0 | 180.3 | 0.5 | 0.8 |
| PBMC + LPS(5 ug/ml) + | 1st | 942.5 | 10.0 | 2807.3 | 4.8 | 584.9 |
| Xiidra 1 uM | 2nd | 670.0 | 10.0 | 1974.8 | 4.4 | 448.8 |
| | Mean | 806.3 | 10.0 | 2391.0 | 4.6 | 516.8 |
| | STDER | 136.3 | 0.0 | 416.3 | 0.2 | 68.0 |
| PBMC + LPS(5 ug/ml) + | 1st | 1003.5 | 10.0 | 2995.2 | 5.8 | 516.4 |
| Xiidra 10 uM | 2nd | 964.0 | 10.0 | 2873.5 | 6.4 | 449.0 |
| | Mean | 983.8 | 10.0 | 2934.4 | 6.1 | 482.7 |
| | STDER | 19.8 | 0.0 | 60.9 | 0.3 | 33.7 |
| PBMC + LPS(5 ug/ml) + | 1st | 802.0 | 10.0 | 2376.5 | 7.1 | 334.7 |
| Xiidra 100 uM | 2nd | 778.0 | 10.0 | 2303.3 | 5.8 | 397.1 |
| | Mean | 790.0 | 10.0 | 2339.9 | 6.5 | 365.9 |
| | STDER | 12.0 | 0.0 | 36.6 | 0.7 | 31.2 |
| PBMC + LPS(5 ug/ml) + | 1st | 854.5 | 10.0 | 2537.1 | 5.7 | 445.1 |
| YY101 0.001 uM | 2nd | 878.0 | 10.0 | 2609.2 | 7.1 | 367.5 |
| | 3rd | 729.0 | 10.0 | 2154.0 | 6.6 | 326.4 |
| | Mean | 820.5 | 10.0 | 2433.4 | 6.5 | 379.7 |
| | STDER | 46.3 | 0.0 | 141.3 | 0.4 | 34.8 |
| PBMC + LPS(5 ug/ml) + | 1st | 946.5 | 10.0 | 2819.6 | 6.4 | 440.6 |
| YY101 0.01 uM | 2nd | 935.0 | 10.0 | 2784.2 | 6.0 | 464.0 |
| | 3rd | 887.0 | 10.0 | 2636.8 | 6.1 | 432.3 |
| | Mean | 922.8 | 10.0 | 2746.9 | 6.2 | 445.6 |
| | STDER | 18.2 | 0.0 | 56.0 | 0.1 | 9.5 |
| PBMC + LPS(5 ug/ml) + | 1st | 937.5 | 10.0 | 2791.9 | 6.2 | 450.3 |
| YY101 0.1 uM | 2nd | 963.0 | 10.0 | 2870.4 | 5.2 | 552.0 |
| | 3rd | 971.0 | 10.0 | 2895.0 | 6.2 | 466.9 |
| | Mean | 957.2 | 10.0 | 2852.4 | 5.9 | 489.7 |
| | STDER | 10.1 | 0.0 | 31.1 | 0.3 | 31.5 |
| PBMC + LPS(5 ug/ml) + | 1st | 920.0 | 10.0 | 2738.1 | 6.3 | 434.6 |
| YY101 1 uM | 2nd | 844.5 | 10.0 | 2506.5 | 7.1 | 353.0 |
| | 3rd | 814.0 | 10.0 | 2413.2 | 6.2 | 389.2 |
| | Mean | 859.5 | 10.0 | 2552.6 | 6.5 | 392.3 |
| | STDER | 31.5 | 0.0 | 96.6 | 0.3 | 23.6 |
| PBMC + LPS(5 ug/ml) + | 1st | 921.0 | 10.0 | 2741.2 | 6.0 | 456.9 |
| YY101 10 uM | 2nd | 942.5 | 10.0 | 2807.3 | 6.4 | 438.6 |
| | 3rd | 905.0 | 10.0 | 2692.0 | 7.6 | 354.2 |
| | Mean | 922.8 | 10.0 | 2746.8 | 6.7 | 416.6 |
| | STDER | 10.9 | 0.0 | 33.4 | 0.5 | 31.6 |
| PBMC + LPS(5 ug/ml) + | 1st | 865.0 | 10.0 | 2569.3 | 6.3 | 407.8 |
| YY101 100 uM | 2nd | 783.0 | 10.0 | 2318.5 | 6.2 | 374.0 |
| | 3rd | 823.5 | 10.0 | 2442.3 | 5.0 | 488.5 |
| | Mean | 823.8 | 10.0 | 2443.4 | 5.8 | 423.4 |
| | STDER | 23.7 | 0.0 | 72.4 | 0.4 | 34.0 |
| PBMC + LPS(5 ug/ml) + | 1st | 427.5 | 10.0 | 1245.3 | 4.5 | 276.7 |
| YDE011 0.001 uM | 2nd | 426.0 | 10.0 | 1240.9 | 5.8 | 213.9 |
| | 3rd | 441.0 | 10.0 | 1285.6 | 5.8 | 221.7 |
| | Mean | 431.5 | 10.0 | 1257.3 | 5.4 | 237.4 |
| | STDER | 4.8 | 0.0 | 14.2 | 0.4 | 19.8 |
| PBMC + LPS(5 ug/ml) + | 1st | 449.0 | 10.0 | 1309.5 | 6.3 | 207.9 |
| YDE011 0.01 uM | 2nd | 441.0 | 10.0 | 1285.6 | 6.2 | 207.4 |
| | 3rd | 441.0 | 10.0 | 1285.6 | 5.1 | 252.1 |
| | Mean | 443.7 | 10.0 | 1293.5 | 5.9 | 222.4 |
| | STDER | 2.7 | 0.0 | 8.0 | 0.4 | 14.8 |
| PBMC + LPS(5 ug/ml) + | 1st | 437.0 | 10.0 | 1273.7 | 6.6 | 193.0 |
| YDE011 0.1 uM | 2nd | 438.0 | 10.0 | 1276.6 | 5.0 | 255.3 |
| | 3rd | 432.0 | 10.0 | 1258.7 | 5.7 | 220.8 |
| | Mean | 435.7 | 10.0 | 1269.7 | 5.8 | 223.0 |
| | STDER | 1.9 | 0.0 | 5.5 | 0.5 | 18.0 |
| PBMC + LPS(5 ug/ml) + | 1st | 440.0 | 10.0 | 1282.6 | 5.5 | 233.2 |
| YDE011 1 uM | 2nd | 415.0 | 10.0 | 1208.1 | 4.8 | 251.7 |
| | 3rd | 439.0 | 10.0 | 1279.6 | 4.8 | 266.6 |
| | Mean | 431.3 | 10.0 | 1256.8 | 5.0 | 250.5 |
| | STDER | 8.2 | 0.0 | 24.4 | 0.2 | 9.7 |
| PBMC + LPS(5 ug/ml) + | 1st | 479.0 | 10.0 | 1399.2 | 5.4 | 259.1 |
| YDE011 10 uM | 2nd | 431.0 | 10.0 | 1255.8 | 5.8 | 216.5 |
| | 3rd | 458.0 | 10.0 | 1336.4 | 4.7 | 284.3 |
| | Mean | 456.0 | 10.0 | 1330.4 | 5.3 | 253.3 |
| | STDER | 13.9 | 0.0 | 41.5 | 0.3 | 19.8 |
| PBMC + LPS(5 ug/ml) + | 1st | 340.0 | 10.0 | 985.6 | 6.1 | 161.6 |
| YDE011 100 uM | 2nd | 331.5 | 10.0 | 960.5 | 6.1 | 157.5 |
| | 3rd | 331.0 | 10.0 | 959.0 | 6.0 | 159.8 |
| | Mean | 334.2 | 10.0 | 968.4 | 6.1 | 159.6 |
| | STDER | 2.9 | 0.0 | 8.6 | 0.0 | 1.2 |
| PBMC + LPS(5 ug/ml) + | 1st | 428.5 | 10.0 | 1248.3 | 4.4 | 283.7 |
| YDE043 0.001 uM | 2nd | 481.0 | 10.0 | 1405.1 | 6.0 | 234.2 |
| | 3rd | 453.0 | 10.0 | 1321.4 | 4.6 | 287.3 |
| | Mean | 454.2 | 10.0 | 1325.0 | 5.0 | 268.4 |
| | STDER | 15.2 | 0.0 | 45.3 | 0.5 | 17.1 |
| PBMC + LPS(5 ug/ml) + | 1st | 440.0 | 10.0 | 1282.6 | 4.5 | 285.0 |
| YDE043 0.01 uM | 2nd | 453.5 | 10.0 | 1322.9 | 6.6 | 200.4 |
| | 3rd | 471.5 | 10.0 | 1376.7 | 5.0 | 275.3 |
| | Mean | 455.0 | 10.0 | 1327.4 | 5.4 | 253.6 |
| | STDER | 9.1 | 0.0 | 27.3 | 0.6 | 26.7 |
| PBMC + LPS(5 ug/ml) + | 1st | 430.5 | 10.0 | 1254.3 | 5.0 | 250.9 |
| YDE043 0.1 uM | 2nd | 457.0 | 10.0 | 1333.4 | 5.6 | 238.1 |
| | 3rd | 469.0 | 10.0 | 1369.2 | 5.6 | 244.5 |
| | Mean | 452.2 | 10.0 | 1319.0 | 5.4 | 244.5 |
| | STDER | 11.4 | 0.0 | 34.0 | 0.2 | 3.7 |
| PBMC + LPS(5 ug/ml) + | 1st | 456.0 | 10.0 | 1330.4 | 5.8 | 229.4 |
| YDE043 1 uM | 2nd | 391.0 | 10.0 | 1136.7 | 4.8 | 236.8 |
| | 3rd | 451.0 | 10.0 | 1315.4 | 5.3 | 248.2 |
| | Mean | 432.7 | 10.0 | 1260.8 | 5.3 | 238.1 |
| | STDER | 20.9 | 0.0 | 62.2 | 0.3 | 5.5 |
| PBMC + LPS(5 ug/ml) + | 1st | 404.0 | 10.0 | 1175.4 | 5.2 | 226.0 |
| YDE043 10 uM | 2nd | 429.0 | 10.0 | 1249.8 | 5.4 | 231.4 |
| | 3rd | 452.0 | 10.0 | 1318.4 | 5.6 | 235.4 |
| | Mean | 428.3 | 10.0 | 1247.9 | 5.4 | 231.0 |
| | STDER | 13.9 | 0.0 | 41.3 | 0.1 | 2.7 |
| PBMC + LPS(5 ug/ml) + | 1st | 330.0 | 10.0 | 956.0 | 6.2 | 154.2 |
| YDE043 100 uM | 2nd | 293.0 | 10.0 | 847.0 | 6.4 | 132.4 |
| | Mean | 311.5 | 10.0 | 901.5 | 6.3 | 143.3 |
| | STDER | 18.5 | 0.0 | 54.5 | 0.1 | 10.9 |

TABLE 31
Bead name: CCL3/MIP-1 alpha

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.5 | 10.0 | NaN | | |
| | 2nd | −0.5 | 10.0 | NaN | | |
| | Mean | 0.0 | 10.0 | #DIV/0! | #DIV/0! | #DIV/0! |
| | STDER | 0.5 | 0.0 | #DIV/0! | #DIV/0! | #DIV/0! |
| | 1st | −0.5 | 10.0 | | 7.5 | 0.0 |
| PBMC + 0.5% DMSO | 2nd | −0.5 | 10.0 | | 7.4 | 0.0 |
| | 3rd | −0.5 | 10.0 | | 6.6 | 0.0 |
| | Mean | −0.5 | 10.0 | #DIV/0! | 7.2 | 0.0 |
| | STDER | 0.0 | 0.0 | #DIV/0! | 0.3 | 0.0 |
| PBMC + LPS(5 ug/ml) | 1st | 2832.5 | 10.0 | 33012.9 | 6.1 | 5411.9 |
| | 2nd | 2469.5 | 10.0 | 30553.0 | 5.8 | 5267.8 |
| | 3rd | 2329.0 | 10.0 | 29577.8 | 5.5 | 5377.8 |
| | Mean | 2543.7 | 10.0 | 31047.9 | 5.8 | 5352.5 |
| | STDER | 150.0 | 0.0 | 1022.0 | 0.2 | 43.5 |
| PBMC + YY101 0.01 uM | 1st | 0.5 | 10.0 | | 5.2 | 0.0 |
| | 2nd | −1.0 | 10.0 | | 4.8 | 0.0 |
| | Mean | −0.3 | 10.0 | #DIV/0! | 5.0 | 0.0 |
| | STDER | 0.8 | 0.0 | #DIV/0! | 0.2 | 0.0 |
| PBMC + YY101 0.1 uM | 1st | −0.5 | 10.0 | | 4.4 | 0.0 |
| | 2nd | −0.5 | 10.0 | | 5.8 | 0.0 |
| | Mean | −0.5 | 10.0 | #DIV/0! | 5.1 | 0.0 |
| | STDER | 0.0 | 0.0 | #DIV/0! | 0.7 | 0.0 |
| PBMC + YY101 1 uM | 1st | 1.5 | 10.0 | 588.0 | 5.5 | 106.9 |
| | 2nd | 0.5 | 10.0 | | 5.5 | 0.0 |
| | Mean | 1.0 | 10.0 | 588.0 | 5.5 | 53.5 |
| | STDER | 0.5 | 0.0 | #DIV/0! | 0.0 | 53.5 |
| PBMC + YY101 10 uM | 1st | 7.5 | 10.0 | 1997.0 | 6.4 | 312.0 |
| | 2nd | −0.5 | 10.0 | | 6.1 | 0.0 |
| | Mean | 3.5 | 10.0 | 1997.0 | 6.3 | 156.0 |
| | STDER | 4.0 | 0.0 | #DIV/0! | 0.2 | 156.0 |
| PBMC + YY101 100 uM | 1st | 2.5 | 10.0 | 1030.2 | 6.4 | 161.0 |
| | 2nd | −0.5 | 10.0 | | 7.0 | 0.0 |
| | Mean | 1.0 | 10.0 | 1030.2 | 6.7 | 80.5 |
| | STDER | 1.5 | 0.0 | #DIV/0! | 0.3 | 80.5 |
| PBMC + LPS(5 ug/ml) + | 1st | 2391.5 | 10.0 | 30013.4 | 6.2 | 4840.9 |
| Xiidra 0.001 uM | 2nd | 2296.5 | 10.0 | 29350.1 | 7.6 | 3861.9 |
| | Mean | 2344.0 | 10.0 | 29681.8 | 6.9 | 4351.4 |
| | STDER | 47.5 | 0.0 | 331.7 | 0.7 | 489.5 |
| PBMC + LPS(5 ug/ml) + | 1st | 2786.0 | 10.0 | 32701.9 | 5.2 | 6288.8 |
| Xiidra 0.01 uM | 2nd | 2444.5 | 10.0 | 30380.5 | 5.8 | 5238.0 |
| | Mean | 2615.3 | 10.0 | 31541.2 | 5.5 | 5763.4 |
| | STDER | 170.8 | 0.0 | 1160.7 | 0.3 | 525.4 |
| PBMC + LPS(5 ug/ml) + | 1st | 2010.0 | 10.0 | 27301.7 | 6.6 | 4136.6 |
| Xiidra 0.1 uM | 2nd | 1672.5 | 10.0 | 24770.7 | 5.7 | 4345.7 |
| | Mean | 1841.3 | 10.0 | 26036.2 | 6.2 | 4241.2 |
| | STDER | 168.8 | 0.0 | 1265.5 | 0.5 | 104.6 |
| PBMC + LPS(5 ug/ml) + | 1st | 1962.5 | 10.0 | 26954.1 | 4.8 | 5615.4 |
| Xiidra 1 uM | 2nd | 1468.5 | 10.0 | 23157.9 | 4.4 | 5263.2 |
| | Mean | 1715.5 | 10.0 | 25056.0 | 4.6 | 5439.3 |
| | STDER | 247.0 | 0.0 | 1898.1 | 0.2 | 176.1 |
| PBMC + LPS(5 ug/ml) + | 1st | 1875.5 | 10.0 | 26310.6 | 5.8 | 4536.3 |
| Xiidra 10 uM | 2nd | 1893.5 | 10.0 | 26444.5 | 6.4 | 4131.9 |
| | Mean | 1884.5 | 10.0 | 26377.5 | 6.1 | 4334.1 |
| | STDER | 9.0 | 0.0 | 66.9 | 0.3 | 202.2 |
| PBMC + LPS(5 ug/ml) + | 1st | 1943.0 | 10.0 | 26810.6 | 7.1 | 3776.1 |
| Xiidra 100 uM | 2nd | 1557.5 | 10.0 | 23870.5 | 5.8 | 4115.6 |
| | Mean | 1750.3 | 10.0 | 25340.6 | 6.5 | 3945.9 |
| | STDER | 192.8 | 0.0 | 1470.0 | 0.7 | 169.7 |
| PBMC + LPS(5 ug/ml) + | 1st | 2104.5 | 10.0 | 27986.1 | 5.7 | 4909.8 |
| YY101 0.001 uM | 2nd | 1995.5 | 10.0 | 27195.8 | 7.1 | 3830.4 |
| | 3rd | 1764.5 | 10.0 | 25475.7 | 6.6 | 3860.0 |
| | Mean | 1954.8 | 10.0 | 26885.9 | 6.5 | 4200.1 |
| | STDER | 100.2 | 0.0 | 741.1 | 0.4 | 355.0 |
| PBMC + LPS(5 ug/ml) + | 1st | 2293.5 | 10.0 | 29329.0 | 6.4 | 4582.7 |
| YY101 0.01 uM | 2nd | 2303.0 | 10.0 | 29395.7 | 6.0 | 4899.3 |
| | 3rd | 2116.5 | 10.0 | 28072.3 | 6.1 | 4602.0 |
| | Mean | 2237.7 | 10.0 | 28932.4 | 6.2 | 4694.7 |
| | STDER | 60.6 | 0.0 | 430.4 | 0.1 | 102.5 |
| PBMC + LPS(5 ug/ml) + | 1st | 2157.0 | 10.0 | 28362.4 | 6.2 | 4574.6 |
| YY101 0.1 uM | 2nd | 2199.5 | 10.0 | 28665.1 | 5.2 | 5512.5 |
| | 3rd | 2154.5 | 10.0 | 28344.5 | 6.2 | 4571.7 |
| | Mean | 2170.3 | 10.0 | 28457.4 | 5.9 | 4886.3 |
| | STDER | 14.6 | 0.0 | 104.0 | 0.3 | 313.1 |
| PBMC + LPS(5 ug/ml) + | 1st | 2206.5 | 10.0 | 28714.8 | 6.3 | 4557.9 |
| YY101 1 uM | 2nd | 2029.0 | 10.0 | 27440.0 | 7.1 | 3864.8 |
| | 3rd | 2109.5 | 10.0 | 28022.0 | 6.2 | 4519.7 |
| | Mean | 2115.0 | 10.0 | 28059.0 | 6.5 | 4314.1 |
| | STDER | 51.3 | 0.0 | 368.5 | 0.3 | 224.9 |
| PBMC + LPS(5 ug/ml) + | 1st | 2406.5 | 10.0 | 30117.5 | 6.0 | 5019.6 |
| YY101 10 uM | 2nd | 2501.0 | 10.0 | 30769.7 | 6.4 | 4807.8 |
| | 3rd | 2251.0 | 10.0 | 29029.8 | 7.6 | 3819.7 |
| | Mean | 2386.2 | 10.0 | 29972.3 | 6.7 | 4549.0 |
| | STDER | 72.9 | 0.0 | 507.5 | 0.5 | 369.7 |
| PBMC + LPS(5 ug/ml) + | 1st | 3190.5 | 10.0 | 35374.6 | 6.3 | 5615.0 |
| YY101 100 uM | 2nd | 2648.5 | 10.0 | 31775.7 | 6.2 | 5125.1 |
| | 3rd | 2798.0 | 10.0 | 32782.2 | 5.0 | 6556.4 |
| | Mean | 2879.0 | 10.0 | 33310.8 | 5.8 | 5765.5 |
| | STDER | 161.6 | 0.0 | 1072.0 | 0.4 | 420.0 |
| PBMC + LPS(5 ug/ml) + | 1st | 134.5 | 10.0 | 7525.9 | 4.5 | 1672.4 |
| YDE011 0.001 uM | 2nd | 126.5 | 10.0 | 7321.9 | 5.8 | 1262.4 |
| | 3rd | 137.0 | 10.0 | 7588.4 | 5.8 | 1308.3 |
| | Mean | 132.7 | 10.0 | 7478.7 | 5.4 | 1414.4 |
| | STDER | 3.2 | 0.0 | 80.5 | 0.4 | 129.7 |
| PBMC + LPS(5 ug/ml) + | 1st | 138.5 | 10.0 | 7625.5 | 6.3 | 1210.4 |
| YDE011 0.01 uM | 2nd | 137.5 | 10.0 | 7600.8 | 6.2 | 1225.9 |
| | 3rd | 134.5 | 10.0 | 7525.9 | 5.1 | 1475.7 |
| | Mean | 136.8 | 10.0 | 7584.1 | 5.9 | 1304.0 |
| | STDER | 1.2 | 0.0 | 29.9 | 0.4 | 86.0 |
| PBMC + LPS(5 ug/ml) + | 1st | 113.5 | 10.0 | 6974.8 | 6.6 | 1056.8 |
| YDE011 0.1 uM | 2nd | 127.0 | 10.0 | 7334.8 | 5.0 | 1467.0 |
| | 3rd | 114.5 | 10.0 | 7002.2 | 5.7 | 1228.5 |
| | Mean | 118.3 | 10.0 | 7103.9 | 5.8 | 1250.7 |
| | STDER | 4.3 | 0.0 | 115.7 | 0.5 | 118.9 |
| PBMC + LPS(5 ug/ml) + | 1st | 129.5 | 10.0 | 7399.2 | 5.5 | 1345.3 |
| YDE011 1 uM | 2nd | 138.0 | 10.0 | 7613.2 | 4.8 | 1586.1 |
| | 3rd | 137.5 | 10.0 | 7600.8 | 4.8 | 1583.5 |
| | Mean | 135.0 | 10.0 | 7537.7 | 5.0 | 1505.0 |
| | STDER | 2.8 | 0.0 | 69.4 | 0.2 | 79.8 |
| PBMC + LPS(5 ug/ml) + | 1st | 152.5 | 10.0 | 7962.5 | 5.4 | 1474.5 |
| YDE011 10 uM | 2nd | 128.5 | 10.0 | 7373.5 | 5.8 | 1271.3 |
| | 3rd | 148.5 | 10.0 | 7868.0 | 4.7 | 1674.0 |
| | Mean | 143.2 | 10.0 | 7734.7 | 5.3 | 1473.3 |
| | STDER | 7.4 | 0.0 | 182.6 | 0.3 | 116.3 |
| PBMC + LPS(5 ug/ml) + | 1st | 164.5 | 10.0 | 8238.4 | 6.1 | 1350.6 |
| YDE011 100 uM | 2nd | 157.0 | 10.0 | 8067.3 | 6.1 | 1322.5 |
| | 3rd | 137.5 | 10.0 | 7600.8 | 6.0 | 1266.8 |
| | Mean | 153.0 | 10.0 | 7968.8 | 6.1 | 1313.3 |
| | STDER | 8.0 | 0.0 | 190.5 | 0.0 | 24.6 |
| PBMC + LPS(5 ug/ml) + | 1st | 139.0 | 10.0 | 7637.9 | 4.4 | 1735.9 |
| YDE043 0.001 uM | 2nd | 163.0 | 10.0 | 8204.6 | 6.0 | 1367.4 |
| | 3rd | 152.5 | 10.0 | 7962.5 | 4.6 | 1731.0 |
| | Mean | 151.5 | 10.0 | 7935.0 | 5.0 | 1611.4 |
| | STDER | 6.9 | 0.0 | 164.2 | 0.5 | 122.0 |
| PBMC + LPS(5 ug/ml) + | 1st | 145.5 | 10.0 | 7796.2 | 4.5 | 1732.5 |
| YDE043 0.01 uM | 2nd | 153.5 | 10.0 | 7985.9 | 6.6 | 1210.0 |
| | 3rd | 148.5 | 10.0 | 7868.0 | 5.0 | 1573.6 |
| | Mean | 149.2 | 10.0 | 7883.4 | 5.4 | 1505.4 |
| | STDER | 2.3 | 0.0 | 55.3 | 0.6 | 154.6 |
| PBMC + LPS(5 ug/ml) + | 1st | 134.0 | 10.0 | 7513.4 | 5.0 | 1502.7 |
| YDE043 0.1 uM | 2nd | 138.5 | 10.0 | 7625.5 | 5.6 | 1361.7 |
| | 3rd | 145.5 | 10.0 | 7796.2 | 5.6 | 1392.2 |
| | Mean | 139.3 | 10.0 | 7645.0 | 5.4 | 1418.9 |
| | STDER | 3.3 | 0.0 | 82.2 | 0.2 | 42.8 |
| PBMC + LPS(5 ug/ml) + | 1st | 152.5 | 10.0 | 7962.5 | 5.8 | 1372.8 |
| YDE043 1 uM | 2nd | 110.5 | 10.0 | 6891.7 | 4.8 | 1435.8 |
| | 3rd | 156.5 | 10.0 | 8055.7 | 5.3 | 1520.0 |
| | Mean | 139.8 | 10.0 | 7636.6 | 5.3 | 1442.9 |
| | STDER | 14.7 | 0.0 | 373.5 | 0.3 | 42.6 |
| PBMC + LPS(5 ug/ml) + | 1st | 133.5 | 10.0 | 7500.8 | 5.2 | 1442.5 |
| YDE043 10 uM | 2nd | 131.5 | 10.0 | 7450.2 | 5.4 | 1379.7 |
| | 3rd | 148.5 | 10.0 | 7868.0 | 5.6 | 1405.0 |
| | Mean | 137.8 | 10.0 | 7606.3 | 5.4 | 1409.0 |
| | STDER | 5.4 | 0.0 | 131.6 | 0.1 | 18.2 |
| PBMC + LPS(5 ug/ml) + | 1st | 152.5 | 10.0 | 7962.5 | 6.2 | 1284.3 |
| YDE043 100 uM | 2nd | 136.5 | 10.0 | 7575.9 | 6.4 | 1183.7 |
| | Mean | 144.5 | 10.0 | 7769.2 | 6.3 | 1234.0 |
| | STDER | 8.0 | 0.0 | 193.3 | 0.1 | 50.3 |

TABLE 32
Bead name: CCL4/MIP-1 beta

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.0 | 10.0 | NaN | | |
| | 2nd | 0.0 | 10.0 | NaN | | |
| | Mean | 0.0 | 10.0 | #DIV/0! | #DIV/0! | #DIV/0! |
| | STDER | 0.0 | 0.0 | #DIV/0! | #DIV/0! | #DIV/0! |
| PBMC + 0.5% DMSO | 1st | 0.0 | 10.0 | | 7.5 | 0.0 |
| | 2nd | 0.0 | 10.0 | | 7.4 | 0.0 |
| | 3rd | 0.0 | 10.0 | | 6.6 | 0.0 |
| | Mean | 0.0 | 10.0 | #DIV/0! | 7.2 | 0.0 |
| | STDER | 0.0 | 0.0 | #DIV/0! | 0.3 | 0.0 |
| PBMC + LPS(5 ug/ml) | 1st | 794.0 | 10.0 | 18382.1 | 6.1 | 3013.5 |
| | 2nd | 724.0 | 10.0 | 17299.3 | 5.8 | 2982.6 |
| | 3rd | 697.5 | 10.0 | 16886.0 | 5.5 | 3070.2 |
| | Mean | 738.5 | 10.0 | 17522.4 | 5.8 | 3022.1 |
| | STDER | 28.8 | 0.0 | 446.1 | 0.2 | 25.6 |
| PBMC + YY101 0.01 uM | 1st | 0.0 | 10.0 | | 5.2 | 0.0 |
| | 2nd | 0.0 | 10.0 | | 4.8 | 0.0 |
| | Mean | 0.0 | 10.0 | #DIV/0! | 5.0 | 0.0 |
| | STDER | 0.0 | 0.0 | #DIV/0! | 0.2 | 0.0 |
| PBMC + YY101 0.1 uM | 1st | 0.0 | 10.0 | | 4.4 | 0.0 |
| | 2nd | 0.0 | 10.0 | | 5.8 | 0.0 |
| | Mean | 0.0 | 10.0 | #DIV/0! | 5.1 | 0.0 |
| | STDER | 0.0 | 0.0 | #DIV/0! | 0.7 | 0.0 |
| PBMC + YY101 1 uM | 1st | −1.0 | 10.0 | | 5.5 | 0.0 |
| | 2nd | −0.5 | 10.0 | | 5.5 | 0.0 |
| | Mean | −0.8 | 10.0 | #DIV/0! | 5.5 | 0.0 |
| | STDER | 0.3 | 0.0 | #DIV/0! | 0.0 | 0.0 |
| PBMC + YY101 10 uM | 1st | 10.0 | 10.0 | 1948.5 | 6.4 | 304.4 |
| | 2nd | 1.0 | 10.0 | | 6.1 | 0.0 |
| | Mean | 5.5 | 10.0 | 1948.5 | 6.3 | 152.2 |
| | STDER | 4.5 | 0.0 | #DIV/0! | 0.2 | 152.2 |
| PBMC + YY101 100 uM | 1st | 10.0 | 10.0 | 1948.5 | 6.4 | 304.4 |
| | 2nd | 3.0 | 10.0 | 772.9 | 7.0 | 110.4 |
| | Mean | 6.5 | 10.0 | 1360.7 | 6.7 | 207.4 |
| | STDER | 3.5 | 0.0 | 587.8 | 0.3 | 97.0 |
| PBMC + LPS(5 ug/ml) + Xiidra 0.001 uM | 1st | 615.0 | 10.0 | 15584.4 | 6.2 | 2513.6 |
| | 2nd | 573.0 | 10.0 | 14911.0 | 7.6 | 1962.0 |
| | Mean | 594.0 | 10.0 | 15247.7 | 6.9 | 2237.8 |
| | STDER | 21.0 | 0.0 | 336.7 | 0.7 | 275.8 |
| PBMC + LPS(5 ug/ml) + Xiidra 0.01 uM | 1st | 700.0 | 10.0 | 16925.1 | 5.2 | 3254.8 |
| | 2nd | 628.0 | 10.0 | 15791.2 | 5.8 | 2722.6 |
| | Mean | 664.0 | 10.0 | 16358.1 | 5.5 | 2988.7 |
| | STDER | 36.0 | 0.0 | 566.9 | 0.3 | 266.1 |
| PBMC + LPS(5 ug/ml) + Xiidra 0.1 uM | 1st | 524.0 | 10.0 | 14113.8 | 6.6 | 2138.5 |
| | 2nd | 442.0 | 10.0 | 12744.4 | 5.7 | 2235.9 |
| | Mean | 483.0 | 10.0 | 13429.1 | 6.2 | 2187.2 |
| | STDER | 41.0 | 0.0 | 684.7 | 0.5 | 48.7 |
| PBMC + LPS(5 ug/ml) + Xiidra 1 uM | 1st | 599.0 | 10.0 | 15328.8 | 4.8 | 3193.5 |
| | 2nd | 397.0 | 10.0 | 11968.5 | 4.4 | 2720.1 |
| | Mean | 498.0 | 10.0 | 13648.6 | 4.6 | 2956.8 |
| | STDER | 101.0 | 0.0 | 1680.2 | 0.2 | 236.7 |
| PBMC + LPS(5 ug/ml) + Xiidra 10 uM | 1st | 630.0 | 10.0 | 15822.9 | 5.8 | 2728.1 |
| | 2nd | 629.0 | 10.0 | 15807.1 | 6.4 | 2469.9 |
| | Mean | 629.5 | 10.0 | 15815.0 | 6.1 | 2599.0 |
| | STDER | 0.5 | 0.0 | 7.9 | 0.3 | 129.1 |
| PBMC + LPS(5 ug/ml) + Xiidra 100 uM | 1st | 631.5 | 10.0 | 15846.7 | 7.1 | 2231.9 |
| | 2nd | 565.5 | 10.0 | 14789.8 | 5.8 | 2550.0 |
| | Mean | 598.5 | 10.0 | 15318.3 | 6.5 | 2391.0 |
| | STDER | 33.0 | 0.0 | 528.5 | 0.7 | 159.0 |
| PBMC + LPS(5 ug/ml) + YY101 0.001 uM | 1st | 551.5 | 10.0 | 14562.9 | 5.7 | 2554.9 |
| | 2nd | 527.0 | 10.0 | 14163.0 | 7.1 | 1994.8 |
| | 3rd | 469.0 | 10.0 | 13200.9 | 6.6 | 2000.1 |
| | Mean | 515.8 | 10.0 | 13975.6 | 6.5 | 2183.3 |
| | STDER | 24.5 | 0.0 | 404.2 | 0.4 | 185.8 |
| PBMC + LPS(5 ug/ml) + YY101 0.01 uM | 1st | 611.0 | 10.0 | 15520.6 | 6.4 | 2425.1 |
| | 2nd | 596.5 | 10.0 | 15288.8 | 6.0 | 2548.1 |
| | 3rd | 560.0 | 10.0 | 14700.8 | 6.1 | 2410.0 |
| | Mean | 589.2 | 10.0 | 15170.0 | 6.2 | 2461.1 |
| | STDER | 15.2 | 0.0 | 244.0 | 0.1 | 43.8 |
| PBMC + LPS(5 ug/ml) + YY101 0.1 uM | 1st | 576.0 | 10.0 | 14959.4 | 6.2 | 2412.8 |
| | 2nd | 592.0 | 10.0 | 15216.6 | 5.2 | 2926.3 |
| | 3rd | 584.0 | 10.0 | 15088.1 | 6.2 | 2433.6 |
| | Mean | 584.0 | 10.0 | 15088.0 | 5.9 | 2590.9 |
| | STDER | 4.6 | 0.0 | 74.3 | 0.3 | 167.8 |
| PBMC + LPS(5 ug/ml) + YY101 1 uM | 1st | 594.0 | 10.0 | 15248.7 | 6.3 | 2420.4 |
| | 2nd | 556.5 | 10.0 | 14644.0 | 7.1 | 2062.5 |
| | 3rd | 547.0 | 10.0 | 14489.7 | 6.2 | 2337.0 |
| | Mean | 565.8 | 10.0 | 14794.1 | 6.5 | 2273.3 |
| | STDER | 14.3 | 0.0 | 231.6 | 0.3 | 108.1 |
| PBMC + LPS(5 ug/ml) + YY101 10 uM | 1st | 614.0 | 10.0 | 15568.4 | 6.0 | 2594.7 |
| | 2nd | 651.0 | 10.0 | 16155.4 | 6.4 | 2524.3 |
| | 3rd | 560.0 | 10.0 | 14700.8 | 7.6 | 1934.3 |
| | Mean | 608.3 | 10.0 | 15474.9 | 6.7 | 2351.1 |
| | STDER | 26.4 | 0.0 | 422.5 | 0.5 | 209.4 |
| PBMC + LPS(5 ug/ml) + YY101 100 uM | 1st | 537.0 | 10.0 | 14326.7 | 6.3 | 2274.1 |
| | 2nd | 474.5 | 10.0 | 13293.2 | 6.2 | 2144.1 |
| | 3rd | 488.0 | 10.0 | 13518.7 | 5.0 | 2703.7 |
| | Mean | 499.8 | 10.0 | 13712.9 | 5.8 | 2374.0 |
| | STDER | 19.0 | 0.0 | 313.7 | 0.4 | 169.1 |
| PBMC + LPS(5 ug/ml) + YDE011 0.001 uM | 1st | 158.0 | 10.0 | 7267.3 | 4.5 | 1615.0 |
| | 2nd | 156.0 | 10.0 | 7220.3 | 5.8 | 1244.9 |
| | 3rd | 158.0 | 10.0 | 7267.3 | 5.8 | 1253.0 |
| | Mean | 157.3 | 10.0 | 7251.7 | 5.4 | 1370.9 |
| | STDER | 0.7 | 0.0 | 15.7 | 0.4 | 122.0 |
| PBMC + LPS(5 ug/ml) + YDE011 0.01 uM | 1st | 167.5 | 10.0 | 7487.6 | 6.3 | 1188.5 |
| | 2nd | 164.0 | 10.0 | 7407.0 | 6.2 | 1194.7 |
| | 3rd | 159.0 | 10.0 | 7290.8 | 5.1 | 1429.6 |
| | Mean | 163.5 | 10.0 | 7395.1 | 5.9 | 1270.9 |
| | STDER | 2.5 | 0.0 | 57.1 | 0.4 | 79.3 |
| PBMC + LPS(5 ug/ml) + YDE011 0.1 uM | 1st | 152.0 | 10.0 | 7125.7 | 6.6 | 1079.6 |
| | 2nd | 151.0 | 10.0 | 7101.9 | 5.0 | 1420.4 |
| | 3rd | 141.0 | 10.0 | 6860.2 | 5.7 | 1203.6 |
| | Mean | 148.0 | 10.0 | 7029.3 | 5.8 | 1234.5 |
| | STDER | 3.5 | 0.0 | 84.8 | 0.5 | 99.6 |
| PBMC + LPS(5 ug/ml) + YDE011 1 uM | 1st | 151.0 | 10.0 | 7101.9 | 5.5 | 1291.2 |
| | 2nd | 152.0 | 10.0 | 7125.7 | 4.8 | 1484.5 |
| | 3rd | 158.0 | 10.0 | 7267.3 | 4.8 | 1514.0 |
| | Mean | 153.7 | 10.0 | 7165.0 | 5.0 | 1429.9 |
| | STDER | 2.2 | 0.0 | 51.7 | 0.2 | 69.9 |
| PBMC + LPS(5 ug/ml) + YDE011 10 uM | 1st | 166.0 | 10.0 | 7453.1 | 5.4 | 1380.2 |
| | 2nd | 153.5 | 10.0 | 7161.3 | 5.8 | 1234.7 |
| | 3rd | 161.5 | 10.0 | 7349.0 | 4.7 | 1563.6 |
| | Mean | 160.3 | 10.0 | 7321.1 | 5.3 | 1392.8 |
| | STDER | 3.7 | 0.0 | 85.4 | 0.3 | 95.2 |
| PBMC + LPS(5 ug/ml) + YDE011 100 uM | 1st | 119.0 | 10.0 | 6303.5 | 6.1 | 1033.4 |
| | 2nd | 121.5 | 10.0 | 6368.7 | 6.1 | 1044.1 |
| | 3rd | 115.0 | 10.0 | 6197.9 | 6.0 | 1033.0 |
| | Mean | 118.5 | 10.0 | 6290.1 | 6.1 | 1036.8 |
| | STDER | 1.9 | 0.0 | 49.8 | 0.0 | 3.6 |
| PBMC + LPS(5 ug/ml) + YDE043 0.001 uM | 1st | 165.5 | 10.0 | 7441.6 | 4.4 | 1691.3 |
| | 2nd | 185.0 | 10.0 | 7881.5 | 6.0 | 1313.6 |
| | 3rd | 182.5 | 10.0 | 7826.1 | 4.6 | 1701.3 |
| | Mean | 177.7 | 10.0 | 7716.4 | 5.0 | 1568.7 |
| | STDER | 6.1 | 0.0 | 138.3 | 0.5 | 127.6 |
| PBMC + LPS(5 ug/ml) + YDE043 0.01 uM | 1st | 178.0 | 10.0 | 7725.6 | 4.5 | 1716.8 |
| | 2nd | 177.0 | 10.0 | 7703.2 | 6.6 | 1167.1 |
| | 3rd | 183.0 | 10.0 | 7837.2 | 5.0 | 1567.4 |
| | Mean | 179.3 | 10.0 | 7755.3 | 5.4 | 1483.8 |
| | STDER | 1.9 | 0.0 | 41.4 | 0.6 | 164.1 |
| PBMC + LPS(5 ug/ml) + YDE043 0.1 uM | 1st | 153.5 | 10.0 | 7161.3 | 5.0 | 1432.3 |
| | 2nd | 159.5 | 10.0 | 7302.4 | 5.6 | 1304.0 |
| | 3rd | 173.0 | 10.0 | 7612.9 | 5.6 | 1359.5 |
| | Mean | 162.0 | 10.0 | 7358.9 | 5.4 | 1365.2 |
| | STDER | 5.8 | 0.0 | 133.4 | 0.2 | 37.1 |
| PBMC + LPS(5 ug/ml) + YDE043 1 uM | 1st | 175.0 | 10.0 | 7658.2 | 5.8 | 1320.4 |
| | 2nd | 137.0 | 10.0 | 6761.7 | 4.8 | 1408.7 |
| | 3rd | 175.0 | 10.0 | 7658.2 | 5.3 | 1444.9 |
| | Mean | 162.3 | 10.0 | 7359.3 | 5.3 | 1391.3 |
| | STDER | 12.7 | 0.0 | 298.8 | 0.3 | 37.0 |
| PBMC + LPS(5 ug/ml) + YDE043 10 uM | 1st | 159.0 | 10.0 | 7290.8 | 5.2 | 1402.1 |
| | 2nd | 158.0 | 10.0 | 7267.3 | 5.4 | 1345.8 |
| | 3rd | 161.0 | 10.0 | 7337.4 | 5.6 | 1310.3 |
| | Mean | 159.3 | 10.0 | 7298.5 | 5.4 | 1352.7 |
| | STDER | 0.9 | 0.0 | 20.6 | 0.1 | 26.7 |
| PBMC + LPS(5 ug/ml) + YDE043 100 uM | 1st | 118.5 | 10.0 | 6290.4 | 6.2 | 1014.6 |
| | 2nd | 104.0 | 10.0 | 5899.5 | 6.4 | 921.8 |
| | Mean | 111.3 | 10.0 | 6094.9 | 6.3 | 968.2 |
| | STDER | 7.3 | 0.0 | 195.4 | 0.1 | 46.4 |

TABLE 33
Bead name: CCL5/RANTES

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.5 | 2.0 | NaN | | |
| | 2nd | −0.5 | 2.0 | NaN | | |
| | Mean | 0.0 | 2.0 | #DIV/0! | #DIV/0! | #DIV/0! |
| | STDER | 0.5 | 0.0 | #DIV/0! | #DIV/0! | #DIV/0! |
| PBMC + 0.5% DMSO | 1st | 49.5 | 2.0 | 35.7 | 7.5 | 4.8 |
| | 2nd | 42.5 | 2.0 | 31.0 | 7.4 | 4.2 |
| | 3rd | 31.0 | 2.0 | 22.5 | 6.6 | 3.4 |
| | Mean | 41.0 | 2.0 | 29.7 | 7.2 | 4.1 |
| | STDER | 5.4 | 0.0 | 3.9 | 0.3 | 0.4 |
| PBMC + LPS(5 ug/ml) | 1st | 1262.5 | 2.0 | 448.6 | 6.1 | 73.5 |
| | 2nd | 1084.5 | 2.0 | 396.8 | 5.8 | 68.4 |
| | 3rd | 1296.0 | 2.0 | 458.2 | 5.5 | 83.3 |
| | Mean | 1214.3 | 2.0 | 434.5 | 5.8 | 75.1 |
| | STDER | 65.6 | 0.0 | 19.1 | 0.2 | 4.4 |
| PBMC + YY101 0.01 uM | 1st | 37.5 | 2.0 | 27.5 | 5.2 | 5.3 |
| | 2nd | 66.5 | 2.0 | 46.1 | 4.8 | 9.6 |
| | Mean | 52.0 | 2.0 | 36.8 | 5.0 | 7.4 |
| | STDER | 14.5 | 0.0 | 9.3 | 0.2 | 2.2 |
| PBMC + YY101 0.1 uM | 1st | 43.5 | 2.0 | 31.7 | 4.4 | 7.2 |
| | 2nd | 57.5 | 2.0 | 40.7 | 5.8 | 7.0 |
| | Mean | 50.5 | 2.0 | 36.2 | 5.1 | 7.1 |
| | STDER | 7.0 | 0.0 | 4.5 | 0.7 | 0.1 |
| PBMC + YY101 1 uM | 1st | 45.5 | 2.0 | 33.0 | 5.5 | 6.0 |
| | 2nd | 52.5 | 2.0 | 37.6 | 5.5 | 6.8 |
| | Mean | 49.0 | 2.0 | 35.3 | 5.5 | 6.4 |
| | STDER | 3.5 | 0.0 | 2.3 | 0.0 | 0.4 |
| PBMC + YY101 10 uM | 1st | 201.5 | 2.0 | 109.6 | 6.4 | 17.1 |
| | 2nd | 70.5 | 2.0 | 48.3 | 6.1 | 7.9 |
| | Mean | 136.0 | 2.0 | 79.0 | 6.3 | 12.5 |
| | STDER | 65.5 | 0.0 | 30.6 | 0.2 | 4.6 |
| PBMC + YY101 100 uM | 1st | 131.5 | 2.0 | 79.2 | 6.4 | 12.4 |
| | 2nd | 82.5 | 2.0 | 55.0 | 7.0 | 7.9 |
| | Mean | 107.0 | 2.0 | 67.1 | 6.7 | 10.1 |
| | STDER | 24.5 | 0.0 | 12.1 | 0.3 | 2.3 |
| PBMC + LPS(5 ug/ml) + Xiidra 0.001 uM | 1st | 1222.5 | 2.0 | 437.0 | 6.2 | 70.5 |
| | 2nd | 1145.5 | 2.0 | 414.6 | 7.6 | 54.6 |
| | Mean | 1184.0 | 2.0 | 425.8 | 6.9 | 62.5 |
| | STDER | 38.5 | 0.0 | 11.2 | 0.7 | 8.0 |
| PBMC + LPS(5 ug/ml) + Xiidra 0.01 uM | 1st | 1212.5 | 2.0 | 434.1 | 5.2 | 83.5 |
| | 2nd | 1054.0 | 2.0 | 387.9 | 5.8 | 66.9 |
| | Mean | 1133.3 | 2.0 | 411.0 | 5.5 | 75.2 |
| | STDER | 79.3 | 0.0 | 23.1 | 0.3 | 8.3 |
| PBMC + LPS(5 ug/ml) + Xiidra 0.1 uM | 1st | 904.5 | 2.0 | 343.6 | 6.6 | 52.1 |
| | 2nd | 898.0 | 2.0 | 341.6 | 5.7 | 59.9 |
| | Mean | 901.3 | 2.0 | 342.6 | 6.2 | 56.0 |
| | STDER | 3.3 | 0.0 | 1.0 | 0.5 | 3.9 |
| PBMC + LPS(5 ug/ml) + Xiidra 1 uM | 1st | 1248.5 | 2.0 | 444.5 | 4.8 | 92.6 |
| | 2nd | 763.5 | 2.0 | 300.9 | 4.4 | 68.4 |
| | Mean | 1006.0 | 2.0 | 372.7 | 4.6 | 80.5 |
| | STDER | 242.5 | 0.0 | 71.8 | 0.2 | 12.1 |
| PBMC + LPS(5 ug/ml) + Xiidra 10 uM | 1st | 959.5 | 2.0 | 360.0 | 5.8 | 62.1 |
| | 2nd | 1078.5 | 2.0 | 395.1 | 6.4 | 61.7 |
| | Mean | 1019.0 | 2.0 | 377.5 | 6.1 | 61.9 |
| | STDER | 59.5 | 0.0 | 17.5 | 0.3 | 0.2 |
| PBMC + LPS(5 ug/ml) + Xiidra 100 uM | 1st | 903.5 | 2.0 | 343.3 | 7.1 | 48.4 |
| | 2nd | 947.5 | 2.0 | 356.4 | 5.8 | 61.5 |
| | Mean | 925.5 | 2.0 | 349.9 | 6.5 | 54.9 |
| | STDER | 22.0 | 0.0 | 6.6 | 0.7 | 6.6 |
| PBMC + LPS(5 ug/ml) + YY101 0.001 uM | 1st | 1120.5 | 2.0 | 407.4 | 5.7 | 71.5 |
| | 2nd | 1139.5 | 2.0 | 412.9 | 7.1 | 58.2 |
| | 3rd | 1049.5 | 2.0 | 386.6 | 6.6 | 58.6 |
| | Mean | 1103.2 | 2.0 | 402.3 | 6.5 | 62.7 |
| | STDER | 27.4 | 0.0 | 8.0 | 0.4 | 4.4 |
| PBMC + LPS(5 ug/ml) + YY101 0.01 uM | 1st | 1358.5 | 2.0 | 476.2 | 6.4 | 74.4 |
| | 2nd | 1246.5 | 2.0 | 443.9 | 6.0 | 74.0 |
| | 3rd | 1240.5 | 2.0 | 442.2 | 6.1 | 72.5 |
| | Mean | 1281.8 | 2.0 | 454.1 | 6.2 | 73.6 |
| | STDER | 38.4 | 0.0 | 11.0 | 0.1 | 0.6 |
| PBMC + LPS(5 ug/ml) + YY101 0.1 uM | 1st | 1206.5 | 2.0 | 432.4 | 6.2 | 69.7 |
| | 2nd | 1188.5 | 2.0 | 427.1 | 5.2 | 82.1 |
| | 3rd | 1179.5 | 2.0 | 424.5 | 6.2 | 68.5 |
| | Mean | 1191.5 | 2.0 | 428.0 | 5.9 | 73.5 |
| | STDER | 7.9 | 0.0 | 2.3 | 0.3 | 4.4 |
| PBMC + LPS(5 ug/ml) + YY101 1 uM | 1st | 1289.0 | 2.0 | 456.2 | 6.3 | 72.4 |
| | 2nd | 1161.5 | 2.0 | 419.3 | 7.1 | 59.1 |
| | 3rd | 1151.5 | 2.0 | 416.4 | 6.2 | 67.2 |
| | Mean | 1200.7 | 2.0 | 430.6 | 6.5 | 66.2 |
| | STDER | 44.3 | 0.0 | 12.8 | 0.3 | 3.9 |
| PBMC + LPS(5 ug/ml) + YY101 10 uM | 1st | 1173.5 | 2.0 | 422.8 | 6.0 | 70.5 |
| | 2nd | 1264.5 | 2.0 | 449.1 | 6.4 | 70.2 |
| | 3rd | 1158.5 | 2.0 | 418.4 | 7.6 | 55.1 |
| | Mean | 1198.8 | 2.0 | 430.1 | 6.7 | 65.2 |
| | STDER | 33.1 | 0.0 | 9.6 | 0.5 | 5.1 |
| PBMC + LPS(5 ug/ml) + YY101 100 uM | 1st | 875.0 | 2.0 | 334.7 | 6.3 | 53.1 |
| | 2nd | 860.5 | 2.0 | 330.4 | 6.2 | 53.3 |
| | 3rd | 909.5 | 2.0 | 345.1 | 5.0 | 69.0 |
| | Mean | 881.7 | 2.0 | 336.7 | 5.8 | 58.5 |
| | STDER | 14.5 | 0.0 | 4.4 | 0.4 | 5.3 |
| PBMC + LPS(5 ug/ml) + YDE011 0.001 uM | 1st | 455.5 | 2.0 | 202.5 | 4.5 | 45.0 |
| | 2nd | 455.5 | 2.0 | 202.5 | 5.8 | 34.9 |
| | 3rd | 520.5 | 2.0 | 224.1 | 5.8 | 38.6 |
| | Mean | 477.2 | 2.0 | 209.7 | 5.4 | 39.5 |
| | STDER | 21.7 | 0.0 | 7.2 | 0.4 | 2.9 |
| PBMC + LPS(5 ug/ml) + YDE011 0.01 uM | 1st | 476.0 | 2.0 | 209.4 | 6.3 | 33.2 |
| | 2nd | 497.5 | 2.0 | 216.5 | 6.2 | 34.9 |
| | 3rd | 508.5 | 2.0 | 220.2 | 5.1 | 43.2 |
| | Mean | 494.0 | 2.0 | 215.3 | 5.9 | 37.1 |
| | STDER | 9.5 | 0.0 | 3.2 | 0.4 | 3.1 |
| PBMC + LPS(5 ug/ml) + YDE011 0.1 uM | 1st | 468.0 | 2.0 | 206.7 | 6.6 | 31.3 |
| | 2nd | 463.5 | 2.0 | 205.2 | 5.0 | 41.0 |
| | 3rd | 475.0 | 2.0 | 209.0 | 5.7 | 36.7 |
| | Mean | 468.8 | 2.0 | 207.0 | 5.8 | 36.3 |
| | STDER | 3.3 | 0.0 | 1.1 | 0.5 | 2.8 |
| PBMC + LPS(5 ug/ml) + YDE011 1 uM | 1st | 464.5 | 2.0 | 205.5 | 5.5 | 37.4 |
| | 2nd | 446.5 | 2.0 | 199.5 | 4.8 | 41.6 |
| | 3rd | 431.5 | 2.0 | 194.4 | 4.8 | 40.5 |
| | Mean | 447.5 | 2.0 | 199.8 | 5.0 | 39.8 |
| | STDER | 9.5 | 0.0 | 3.2 | 0.2 | 1.3 |
| PBMC + LPS(5 ug/ml) + YDE011 10 uM | 1st | 492.0 | 2.0 | 214.7 | 5.4 | 39.8 |
| | 2nd | 453.5 | 2.0 | 201.8 | 5.8 | 34.8 |
| | 3rd | 442.5 | 2.0 | 198.1 | 4.7 | 42.1 |
| | Mean | 462.7 | 2.0 | 204.9 | 5.3 | 38.9 |
| | STDER | 15.0 | 0.0 | 5.0 | 0.3 | 2.2 |
| PBMC + LPS(5 ug/ml) + YDE011 100 uM | 1st | 362.5 | 2.0 | 170.4 | 6.1 | 27.9 |
| | 2nd | 315.5 | 2.0 | 153.5 | 6.1 | 25.2 |
| | 3rd | 337.5 | 2.0 | 161.5 | 6.0 | 26.9 |
| | Mean | 338.5 | 2.0 | 161.8 | 6.1 | 26.7 |
| | STDER | 13.6 | 0.0 | 4.9 | 0.0 | 0.8 |
| PBMC + LPS(5 ug/ml) + YDE043 0.001 uM | 1st | 445.5 | 2.0 | 199.1 | 4.4 | 45.3 |
| | 2nd | 497.5 | 2.0 | 216.5 | 6.0 | 36.1 |
| | 3rd | 465.0 | 2.0 | 205.7 | 4.6 | 44.7 |
| | Mean | 469.3 | 2.0 | 207.1 | 5.0 | 42.0 |
| | STDER | 15.2 | 0.0 | 5.1 | 0.5 | 3.0 |
| PBMC + LPS(5 ug/ml) + YDE043 0.01 uM | 1st | 506.0 | 2.0 | 219.3 | 4.5 | 48.7 |
| | 2nd | 486.5 | 2.0 | 212.9 | 6.6 | 32.3 |
| | 3rd | 483.0 | 2.0 | 211.7 | 5.0 | 42.3 |
| | Mean | 491.8 | 2.0 | 214.6 | 5.4 | 41.1 |
| | STDER | 7.2 | 0.0 | 2.4 | 0.6 | 4.8 |
| PBMC + LPS(5 ug/ml) + YDE043 0.1 uM | 1st | 508.5 | 2.0 | 220.2 | 5.0 | 44.0 |
| | 2nd | 448.5 | 2.0 | 200.1 | 5.6 | 35.7 |
| | 3rd | 530.5 | 2.0 | 227.4 | 5.6 | 40.6 |
| | Mean | 495.8 | 2.0 | 215.9 | 5.4 | 40.1 |
| | STDER | 24.5 | 0.0 | 8.1 | 0.2 | 2.4 |
| PBMC + LPS(5 ug/ml) + YDE043 1 uM | 1st | 502.5 | 2.0 | 218.2 | 5.8 | 37.6 |
| | 2nd | 486.5 | 2.0 | 212.9 | 4.8 | 44.3 |
| | 3rd | 484.5 | 2.0 | 212.2 | 5.3 | 40.0 |
| | Mean | 491.2 | 2.0 | 214.4 | 5.3 | 40.7 |
| | STDER | 5.7 | 0.0 | 1.9 | 0.3 | 2.0 |
| PBMC + LPS(5 ug/ml) + YDE043 10 uM | 1st | 486.5 | 2.0 | 212.9 | 5.2 | 40.9 |
| | 2nd | 429.5 | 2.0 | 193.7 | 5.4 | 35.9 |
| | 3rd | 453.5 | 2.0 | 201.8 | 5.6 | 36.0 |
| | Mean | 456.5 | 2.0 | 202.8 | 5.4 | 37.6 |
| | STDER | 16.5 | 0.0 | 5.6 | 0.1 | 1.7 |
| PBMC + LPS(5 ug/ml) + YDE043 100 uM | 1st | 353.0 | 2.0 | 167.0 | 6.2 | 26.9 |
| | 2nd | 330.5 | 2.0 | 159.0 | 6.4 | 24.8 |
| | Mean | 341.8 | 2.0 | 163.0 | 6.3 | 25.9 |
| | STDER | 11.3 | 0.0 | 4.0 | 0.1 | 1.1 |

TABLE 34
Bead name: IFN-gamma

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.0 | 2.0 | NaN | | |
| | 2nd | 0.0 | 2.0 | NaN | | |
| | Mean | 0.0 | 2.0 | #DIV/0! | #DIV/0! | #DIV/0! |
| | STDER | 0.0 | 0.0 | #DIV/0! | #DIV/0! | #DIV/0! |
| PBMC + 0.5% DMSO | 1st | 1.0 | 2.0 | | 7.5 | 0.0 |
| | 2nd | 1.0 | 2.0 | | 7.4 | 0.0 |
| | 3rd | 0.0 | 2.0 | | 6.6 | 0.0 |
| | Mean | 0.7 | 2.0 | #DIV/0! | 7.2 | 0.0 |
| | STDER | 0.3 | 0.0 | #DIV/0! | 0.3 | 0.0 |
| PBMC + LPS(5 ug/ml) | 1st | 473.0 | 2.0 | 1833.4 | 6.1 | 300.6 |
| | 2nd | 476.0 | 2.0 | 1844.0 | 5.8 | 317.9 |
| | 3rd | 552.5 | 2.0 | 2113.7 | 5.5 | 384.3 |
| | Mean | 500.5 | 2.0 | 1930.4 | 5.8 | 334.3 |
| | STDER | 26.0 | 0.0 | 91.7 | 0.2 | 25.5 |
| PBMC + YY101 0.01 uM | 1st | 5.0 | 2.0 | 12.5 | 5.2 | 2.4 |
| | 2nd | 8.0 | 2.0 | 29.1 | 4.8 | 6.1 |
| | Mean | 6.5 | 2.0 | 20.8 | 5.0 | 4.2 |
| | STDER | 1.5 | 0.0 | 8.3 | 0.2 | 1.8 |
| PBMC + YY101 0.1 uM | 1st | 5.0 | 2.0 | 12.5 | 4.4 | 2.8 |
| | 2nd | 8.0 | 2.0 | 29.1 | 5.8 | 5.0 |
| | Mean | 6.5 | 2.0 | 20.8 | 5.1 | 3.9 |
| | STDER | 1.5 | 0.0 | 8.3 | 0.7 | 1.1 |
| PBMC + YY101 1 uM | 1st | 5.0 | 2.0 | 12.5 | 5.5 | 2.3 |
| | 2nd | 7.0 | 2.0 | 23.7 | 5.5 | 4.3 |
| | Mean | 6.0 | 2.0 | 18.1 | 5.5 | 3.3 |
| | STDER | 1.0 | 0.0 | 5.6 | 0.0 | 1.0 |
| PBMC + YY101 10 uM | 1st | 15.5 | 2.0 | 67.3 | 6.4 | 10.5 |
| | 2nd | 9.0 | 2.0 | 34.4 | 6.1 | 5.6 |
| | Mean | 12.3 | 2.0 | 50.9 | 6.3 | 8.1 |
| | STDER | 3.3 | 0.0 | 16.5 | 0.2 | 2.4 |
| PBMC + YY101 100 uM | 1st | 12.0 | 2.0 | 49.9 | 6.4 | 7.8 |
| | 2nd | 7.0 | 2.0 | 23.7 | 7.0 | 3.4 |
| | Mean | 9.5 | 2.0 | 36.8 | 6.7 | 5.6 |
| | STDER | 2.5 | 0.0 | 13.1 | 0.3 | 2.2 |
| PBMC + LPS(5 ug/ml) + Xiidra 0.001 uM | 1st | 635.0 | 2.0 | 2400.7 | 6.2 | 387.2 |
| | 2nd | 634.5 | 2.0 | 2399.0 | 7.6 | 315.7 |
| | Mean | 634.8 | 2.0 | 2399.8 | 6.9 | 351.4 |
| | STDER | 0.3 | 0.0 | 0.9 | 0.7 | 35.8 |
| PBMC + LPS(5 ug/ml) + Xiidra 0.01 uM | 1st | 690.0 | 2.0 | 2590.1 | 5.2 | 498.1 |
| | 2nd | 498.5 | 2.0 | 1923.7 | 5.8 | 331.7 |
| | Mean | 594.3 | 2.0 | 2256.9 | 5.5 | 414.9 |
| | STDER | 95.8 | 0.0 | 333.2 | 0.3 | 83.2 |
| PBMC + LPS(5 ug/ml) + Xiidra 0.1 uM | 1st | 360.0 | 2.0 | 1427.4 | 6.6 | 216.3 |
| | 2nd | 428.0 | 2.0 | 1672.9 | 5.7 | 293.5 |
| | Mean | 394.0 | 2.0 | 1550.1 | 6.2 | 254.9 |
| | STDER | 34.0 | 0.0 | 122.8 | 0.5 | 38.6 |
| PBMC + LPS(5 ug/ml) + Xiidra 1 uM | 1st | 344.0 | 2.0 | 1369.0 | 4.8 | 285.2 |
| | 2nd | 285.0 | 2.0 | 1151.6 | 4.4 | 261.7 |
| | Mean | 314.5 | 2.0 | 1260.3 | 4.6 | 273.5 |
| | STDER | 29.5 | 0.0 | 108.7 | 0.2 | 11.7 |
| PBMC + LPS(5 ug/ml) + Xiidra 10 uM | 1st | 208.0 | 2.0 | 861.4 | 5.8 | 148.5 |
| | 2nd | 269.0 | 2.0 | 1092.0 | 6.4 | 170.6 |
| | Mean | 238.5 | 2.0 | 976.7 | 6.1 | 159.6 |
| | STDER | 30.5 | 0.0 | 115.3 | 0.3 | 11.0 |
| PBMC + LPS(5 ug/ml) + Xiidra 100 uM | 1st | 123.0 | 2.0 | 529.0 | 7.1 | 74.5 |
| | 2nd | 146.0 | 2.0 | 620.6 | 5.8 | 107.0 |
| | Mean | 134.5 | 2.0 | 574.8 | 6.5 | 90.8 |
| | STDER | 11.5 | 0.0 | 45.8 | 0.7 | 16.2 |
| PBMC + LPS(5 ug/ml) + YY101 0.001 uM | 1st | 619.0 | 2.0 | 2345.3 | 5.7 | 411.5 |
| | 2nd | 724.0 | 2.0 | 2706.6 | 7.1 | 381.2 |
| | 3rd | 596.0 | 2.0 | 2265.5 | 6.6 | 343.3 |
| | Mean | 646.3 | 2.0 | 2439.1 | 6.5 | 378.6 |
| | STDER | 39.4 | 0.0 | 135.7 | 0.4 | 19.7 |
| PBMC + LPS(5 ug/ml) + YY101 0.01 uM | 1st | 940.0 | 2.0 | 3435.7 | 6.4 | 536.8 |
| | 2nd | 906.0 | 2.0 | 3322.0 | 6.0 | 553.7 |
| | 3rd | 705.0 | 2.0 | 2641.6 | 6.1 | 433.0 |
| | Mean | 850.3 | 2.0 | 3133.1 | 6.2 | 507.8 |
| | STDER | 73.3 | 0.0 | 247.9 | 0.1 | 37.7 |
| PBMC + LPS(5 ug/ml) + YY101 0.1 uM | 1st | 527.0 | 2.0 | 2024.2 | 6.2 | 326.5 |
| | 2nd | 408.0 | 2.0 | 1601.1 | 5.2 | 307.9 |
| | 3rd | 491.5 | 2.0 | 1899.0 | 6.2 | 306.3 |
| | Mean | 475.5 | 2.0 | 1841.4 | 5.9 | 313.6 |
| | STDER | 35.3 | 0.0 | 125.5 | 0.3 | 6.5 |
| PBMC + LPS(5 ug/ml) + YY101 1 uM | 1st | 658.0 | 2.0 | 2480.1 | 6.3 | 393.7 |
| | 2nd | 645.5 | 2.0 | 2437.0 | 7.1 | 343.2 |
| | 3rd | 531.0 | 2.0 | 2038.3 | 6.2 | 328.8 |
| | Mean | 611.5 | 2.0 | 2318.4 | 6.5 | 355.2 |
| | STDER | 40.4 | 0.0 | 140.6 | 0.3 | 19.7 |
| PBMC + LPS(5 ug/ml) + YY101 10 uM | 1st | 827.5 | 2.0 | 3058.1 | 6.0 | 509.7 |
| | 2nd | 691.0 | 2.0 | 2593.6 | 6.4 | 405.2 |
| | 3rd | 609.5 | 2.0 | 2312.4 | 7.6 | 304.3 |
| | Mean | 709.3 | 2.0 | 2654.7 | 6.7 | 406.4 |
| | STDER | 63.6 | 0.0 | 217.4 | 0.5 | 59.3 |
| PBMC + LPS(5 ug/ml) + YY101 100 uM | 1st | 504.0 | 2.0 | 1943.2 | 6.3 | 308.4 |
| | 2nd | 555.0 | 2.0 | 2122.4 | 6.2 | 342.3 |
| | 3rd | 450.0 | 2.0 | 1751.5 | 5.0 | 350.3 |
| | Mean | 503.0 | 2.0 | 1939.0 | 5.8 | 333.7 |
| | STDER | 30.3 | 0.0 | 107.1 | 0.4 | 12.8 |
| PBMC + LPS(5 ug/ml) + YDE011 0.001 uM | 1st | 147.0 | 2.0 | 624.6 | 4.5 | 138.8 |
| | 2nd | 174.0 | 2.0 | 730.3 | 5.8 | 125.9 |
| | 3rd | 235.0 | 2.0 | 964.1 | 5.8 | 166.2 |
| | Mean | 185.3 | 2.0 | 773.0 | 5.4 | 143.6 |
| | STDER | 26.0 | 0.0 | 100.3 | 0.4 | 11.9 |
| PBMC + LPS(5 ug/ml) + YDE011 0.01 uM | 1st | 205.0 | 2.0 | 849.9 | 6.3 | 134.9 |
| | 2nd | 185.0 | 2.0 | 773.0 | 6.2 | 124.7 |
| | 3rd | 180.0 | 2.0 | 753.6 | 5.1 | 147.8 |
| | Mean | 190.0 | 2.0 | 792.2 | 5.9 | 135.8 |
| | STDER | 7.6 | 0.0 | 29.4 | 0.4 | 6.7 |
| PBMC + LPS(5 ug/ml) + YDE011 0.1 uM | 1st | 216.0 | 2.0 | 892.0 | 6.6 | 135.1 |
| | 2nd | 180.0 | 2.0 | 753.6 | 5.0 | 150.7 |
| | 3rd | 190.0 | 2.0 | 792.3 | 5.7 | 139.0 |
| | Mean | 195.3 | 2.0 | 812.6 | 5.8 | 141.6 |
| | STDER | 10.7 | 0.0 | 41.2 | 0.5 | 4.7 |
| PBMC + LPS(5 ug/ml) + YDE011 1 uM | 1st | 268.0 | 2.0 | 1088.2 | 5.5 | 197.9 |
| | 2nd | 220.5 | 2.0 | 909.1 | 4.8 | 189.4 |
| | 3rd | 235.0 | 2.0 | 964.1 | 4.8 | 200.9 |
| | Mean | 241.2 | 2.0 | 987.2 | 5.0 | 196.0 |
| | STDER | 14.1 | 0.0 | 53.0 | 0.2 | 3.4 |
| PBMC + LPS(5 ug/ml) + YDE011 10 uM | 1st | 237.5 | 2.0 | 973.6 | 5.4 | 180.3 |
| | 2nd | 208.0 | 2.0 | 861.4 | 5.8 | 148.5 |
| | 3rd | 163.5 | 2.0 | 689.4 | 4.7 | 146.7 |
| | Mean | 203.0 | 2.0 | 841.5 | 5.3 | 158.5 |
| | STDER | 21.5 | 0.0 | 82.6 | 0.3 | 10.9 |
| PBMC + LPS(5 ug/ml) + YDE011 100 uM | 1st | 142.0 | 2.0 | 604.8 | 6.1 | 99.1 |
| | 2nd | 114.0 | 2.0 | 492.8 | 6.1 | 80.8 |
| | 3rd | 117.0 | 2.0 | 504.9 | 6.0 | 84.1 |
| | Mean | 124.3 | 2.0 | 534.1 | 6.1 | 88.0 |
| | STDER | 8.9 | 0.0 | 35.5 | 0.0 | 5.6 |
| PBMC + LPS(5 ug/ml) + YDE043 0.001 uM | 1st | 208.5 | 2.0 | 863.3 | 4.4 | 196.2 |
| | 2nd | 230.0 | 2.0 | 945.2 | 6.0 | 157.5 |
| | 3rd | 185.5 | 2.0 | 774.9 | 4.6 | 168.5 |
| | Mean | 208.0 | 2.0 | 861.2 | 5.0 | 174.1 |
| | STDER | 12.8 | 0.0 | 49.2 | 0.5 | 11.5 |
| PBMC + LPS(5 ug/ml) + YDE043 0.01 uM | 1st | 207.0 | 2.0 | 857.6 | 4.5 | 190.6 |
| | 2nd | 190.0 | 2.0 | 792.3 | 6.6 | 120.0 |
| | 3rd | 211.5 | 2.0 | 874.8 | 5.0 | 175.0 |
| | Mean | 202.8 | 2.0 | 841.6 | 5.4 | 161.9 |
| | STDER | 6.5 | 0.0 | 25.1 | 0.6 | 21.4 |
| PBMC + LPS(5 ug/ml) + YDE043 0.1 uM | 1st | 216.0 | 2.0 | 892.0 | 5.0 | 178.4 |
| | 2nd | 148.0 | 2.0 | 628.5 | 5.6 | 112.2 |
| | 3rd | 211.0 | 2.0 | 872.9 | 5.6 | 155.9 |
| | Mean | 191.7 | 2.0 | 797.8 | 5.4 | 148.8 |
| | STDER | 21.9 | 0.0 | 84.8 | 0.2 | 19.4 |
| PBMC + LPS(5 ug/ml) + YDE043 1 uM | 1st | 175.0 | 2.0 | 734.2 | 5.8 | 126.6 |
| | 2nd | 201.0 | 2.0 | 834.6 | 4.8 | 173.9 |
| | 3rd | 241.5 | 2.0 | 988.7 | 5.3 | 186.5 |
| | Mean | 205.8 | 2.0 | 852.5 | 5.3 | 162.3 |
| | STDER | 19.3 | 0.0 | 74.0 | 0.3 | 18.2 |
| PBMC + LPS(5 ug/ml) + YDE043 10 uM | 1st | 199.5 | 2.0 | 828.9 | 5.2 | 159.4 |
| | 2nd | 199.0 | 2.0 | 826.9 | 5.4 | 153.1 |
| | 3rd | 209.0 | 2.0 | 865.3 | 5.6 | 154.5 |
| | Mean | 202.5 | 2.0 | 840.3 | 5.4 | 155.7 |
| | STDER | 3.3 | 0.0 | 12.5 | 0.1 | 1.9 |
| PBMC + LPS(5 ug/ml) + YDE043 100 uM | 1st | 111.0 | 2.0 | 480.6 | 6.2 | 77.5 |
| | 2nd | 102.0 | 2.0 | 444.0 | 6.4 | 69.4 |
| | Mean | 106.5 | 2.0 | 462.3 | 6.3 | 73.4 |
| | STDER | 4.5 | 0.0 | 18.3 | 0.1 | 4.1 |

TABLE 35
Bead name: CCL2/JE/MCP-1

| | | | | Con- | Live | conc/ |
|---|---|---|---|---|---|---|
| | | | | cen- | cell | live |
| | | | Dilu- | tration | (10\hat{ }5/ | cell (pg/ |
| Sample | | MFI | tion | (pg/ml) | ml) | 10\hat{ }5) |
| Blank | 1st | 0.0 | 2.0 | NaN | | |
| | 2nd | 0.0 | 2.0 | NaN | | |
| | Mean | 0.0 | 2.0 | #DIV/0! | #DIV/0! | #DIV/0! |
| | STDER | 0.0 | 0.0 | #DIV/0! | #DIV/0! | #DIV/0! |
| PBMC + | 1st | 56.0 | 2.0 | 109.1 | 7.5 | 14.5 |
| 0.5% DMSO | 2nd | 176.0 | 2.0 | 202.1 | 7.4 | 27.3 |
| | 3rd | 181.0 | 2.0 | 205.3 | 6.6 | 31.1 |
| | Mean | 137.7 | 2.0 | 172.2 | 7.2 | 24.3 |
| | STDER | 40.9 | 0.0 | 31.5 | 0.3 | 5.0 |
| PBMC + | 1st | 10850.5 | 2.0 | 3125.9 | 6.1 | 512.4 |
| LPS(5 ug/ml) | 2nd | 10117.0 | 2.0 | 2900.2 | 5.8 | 500.0 |
| | 3rd | 10150.0 | 2.0 | 2910.1 | 5.5 | 529.1 |
| | Mean | 10372.5 | 2.0 | 2978.7 | 5.8 | 513.9 |
| | STDER | 239.2 | 0.0 | 73.7 | 0.2 | 8.4 |
| PBMC + | 1st | 4825.5 | 2.0 | 1522.2 | 5.2 | 292.7 |
| YY101 | 2nd | 16019.0 | 2.0 | 5242.2 | 4.8 | 1092.1 |
| 0.01 uM | Mean | 10422.3 | 2.0 | 3382.2 | 5.0 | 692.4 |
| | STDER | 5596.8 | 0.0 | 1860.0 | 0.2 | 399.7 |
| PBMC + | 1st | 4052.5 | 2.0 | 1338.6 | 4.4 | 304.2 |
| YY101 0.1 uM | 2nd | 9626.0 | 2.0 | 2755.6 | 5.8 | 475.1 |
| | Mean | 6839.3 | 2.0 | 2047.1 | 5.1 | 389.7 |
| | STDER | 2786.8 | 0.0 | 708.5 | 0.7 | 85.4 |
| PBMC + | 1st | 4611.0 | 2.0 | 1471.3 | 5.5 | 267.5 |
| YY101 1 uM | 2nd | 7230.0 | 2.0 | 2108.8 | 5.5 | 383.4 |
| | Mean | 5920.5 | 2.0 | 1790.1 | 5.5 | 325.5 |
| | STDER | 1309.5 | 0.0 | 318.8 | 0.0 | 58.0 |
| PBMC + | 1st | 22755.0 | 2.0 | 12584.2 | 6.4 | 1966.3 |
| YY101 10 uM | 2nd | 10032.5 | 2.0 | 2874.9 | 6.1 | 471.3 |
| | Mean | 16393.8 | 2.0 | 7729.5 | 6.3 | 1218.8 |
| | STDER | 6361.3 | 0.0 | 4854.6 | 0.2 | 747.5 |
| PBMC + | 1st | 4286.0 | 2.0 | 1394.1 | 6.4 | 217.8 |
| YY101 100 uM | 2nd | 1867.5 | 2.0 | 796.7 | 7.0 | 113.8 |
| | Mean | 3076.8 | 2.0 | 1095.4 | 6.7 | 165.8 |
| | STDER | 1209.3 | 0.0 | 298.7 | 0.3 | 52.0 |
| PBMC + | 1st | 14536.5 | 2.0 | 4509.4 | 6.2 | 727.3 |
| LPS(5 ug/ml) + | 2nd | 13875.0 | 2.0 | 4222.1 | 7.6 | 555.5 |
| Xiidra | Mean | 14205.8 | 2.0 | 4365.8 | 6.9 | 641.4 |
| 0.001 uM | STDER | 330.8 | 0.0 | 143.7 | 0.7 | 85.9 |
| PBMC + | 1st | 12993.5 | 2.0 | 3869.5 | 5.2 | 744.1 |
| LPS(5 ug/ml) + | 2nd | 14776.0 | 2.0 | 4619.0 | 5.8 | 796.4 |
| Xiidra 0.01 uM | Mean | 13884.8 | 2.0 | 4244.3 | 5.5 | 770.3 |
| | STDER | 891.3 | 0.0 | 374.7 | 0.3 | 26.1 |
| PBMC + | 1st | 10785.0 | 2.0 | 3105.2 | 6.6 | 470.5 |
| LPS(5 ug/ml) + | 2nd | 10732.0 | 2.0 | 3088.6 | 5.7 | 541.9 |
| Xiidra 0.1 uM | Mean | 10758.5 | 2.0 | 3096.9 | 6.2 | 506.2 |
| | STDER | 26.5 | 0.0 | 8.3 | 0.5 | 35.7 |
| PBMC + | 1st | 10830.0 | 2.0 | 3119.4 | 4.8 | 649.9 |
| LPS(5 ug/ml) + | 2nd | 11231.0 | 2.0 | 3248.1 | 4.4 | 738.2 |
| Xiidra 1 uM | Mean | 11030.5 | 2.0 | 3183.8 | 4.6 | 694.0 |
| | STDER | 200.5 | 0.0 | 64.4 | 0.2 | 44.2 |
| PBMC + | 1st | 8984.0 | 2.0 | 2573.5 | 5.8 | 443.7 |
| LPS(5 ug/ml) + | 2nd | 10594.0 | 2.0 | 3045.6 | 6.4 | 475.9 |
| Xiidra 10 uM | Mean | 9789.0 | 2.0 | 2809.5 | 6.1 | 459.8 |
| | STDER | 805.0 | 0.0 | 236.0 | 0.3 | 16.1 |
| PBMC + | 1st | 8030.0 | 2.0 | 2315.5 | 7.1 | 326.1 |
| LPS(5 ug/ml) + | 2nd | 8710.5 | 2.0 | 2498.1 | 5.8 | 430.7 |
| Xiidra 100 uM | Mean | 8370.3 | 2.0 | 2406.8 | 6.5 | 378.4 |
| | STDER | 340.3 | 0.0 | 91.3 | 0.7 | 52.3 |
| PBMC + | 1st | 12717.0 | 2.0 | 3765.3 | 5.7 | 660.6 |
| LPS(5 ug/ml) + | 2nd | 12912.0 | 2.0 | 3838.5 | 7.1 | 540.6 |
| YY101 | 3rd | 13837.0 | 2.0 | 4206.2 | 6.6 | 637.3 |
| 0.001 uM | Mean | 13155.3 | 2.0 | 3936.7 | 6.5 | 612.8 |
| | STDER | 345.5 | 0.0 | 136.4 | 0.4 | 36.7 |
| PBMC + | 1st | 13686.0 | 2.0 | 4143.7 | 6.4 | 647.5 |
| LPS(5 ug/ml) + | 2nd | 13444.5 | 2.0 | 4045.9 | 6.0 | 674.3 |
| YY101 | 3rd | 13067.0 | 2.0 | 3897.7 | 6.1 | 639.0 |
| 0.01 uM | Mean | 13399.2 | 2.0 | 4029.1 | 6.2 | 653.6 |
| | STDER | 180.1 | 0.0 | 71.5 | 0.1 | 10.7 |
| PBMC + | 1st | 12267.0 | 2.0 | 3601.3 | 6.2 | 580.9 |
| LPS(5 ug/ml) + | 2nd | 12562.0 | 2.0 | 3708.0 | 5.2 | 713.1 |
| YY101 0.1 uM | 3rd | 11811.0 | 2.0 | 3442.0 | 6.2 | 555.2 |
| | Mean | 12213.3 | 2.0 | 3583.8 | 5.9 | 616.4 |
| | STDER | 218.4 | 0.0 | 77.3 | 0.3 | 48.9 |
| PBMC + | 1st | 13078.5 | 2.0 | 3902.2 | 6.3 | 619.4 |
| LPS(5 ug/ml) + | 2nd | 13103.0 | 2.0 | 3911.6 | 7.1 | 550.9 |
| YY101 1 uM | 3rd | 13960.0 | 2.0 | 4257.8 | 6.2 | 686.7 |
| | Mean | 13380.5 | 2.0 | 4023.9 | 6.5 | 619.0 |
| | STDER | 289.8 | 0.0 | 117.0 | 0.3 | 39.2 |
| PBMC + | 1st | 11415.0 | 2.0 | 3308.6 | 6.0 | 551.4 |
| LPS(5 ug/ml) + | 2nd | 12533.0 | 2.0 | 3697.4 | 6.4 | 577.7 |
| YY101 10 uM | 3rd | 11609.0 | 2.0 | 3373.4 | 7.6 | 443.9 |
| | Mean | 11852.3 | 2.0 | 3459.8 | 6.7 | 524.3 |
| | STDER | 344.9 | 0.0 | 120.3 | 0.5 | 40.9 |
| PBMC + | 1st | 992.0 | 2.0 | 541.0 | 6.3 | 85.9 |
| LPS(5 ug/ml) + | 2nd | 1275.0 | 2.0 | 629.1 | 6.2 | 101.5 |
| YY101 100 uM | 3rd | 1263.0 | 2.0 | 625.5 | 5.0 | 125.1 |
| | Mean | 1176.7 | 2.0 | 598.6 | 5.8 | 104.2 |
| | STDER | 92.4 | 0.0 | 28.8 | 0.4 | 11.4 |
| PBMC + | 1st | 6216.5 | 2.0 | 1856.7 | 4.5 | 412.6 |
| LPS(5 ug/ml) + | 2nd | 6415.5 | 2.0 | 1905.5 | 5.8 | 328.5 |
| YDE011 | 3rd | 6318.0 | 2.0 | 1881.6 | 5.8 | 324.4 |
| 0.001 uM | Mean | 6316.7 | 2.0 | 1881.3 | 5.4 | 355.2 |
| | STDER | 57.5 | 0.0 | 14.1 | 0.4 | 28.7 |
| PBMC + | 1st | 6375.0 | 2.0 | 1895.6 | 6.3 | 300.9 |
| LPS(5 ug/ml) + | 2nd | 7050.0 | 2.0 | 2063.3 | 6.2 | 332.8 |
| YDE011 | 3rd | 6660.0 | 2.0 | 1965.9 | 5.1 | 385.5 |
| 0.01 uM | Mean | 6695.0 | 2.0 | 1974.9 | 5.9 | 339.7 |
| | STDER | 195.6 | 0.0 | 48.6 | 0.4 | 24.7 |
| PBMC + | 1st | 5566.0 | 2.0 | 1699.1 | 6.6 | 257.4 |
| LPS(5 ug/ml) + | 2nd | 5817.0 | 2.0 | 1759.6 | 5.0 | 351.9 |
| YDE011 | 3rd | 5428.0 | 2.0 | 1666.0 | 5.7 | 292.3 |
| 0.1 uM | Mean | 5603.7 | 2.0 | 1708.3 | 5.8 | 300.6 |
| | STDER | 113.9 | 0.0 | 27.4 | 0.5 | 27.6 |
| PBMC + | 1st | 5965.5 | 2.0 | 1795.6 | 5.5 | 326.5 |
| LPS(5 ug/ml) + | 2nd | 6315.0 | 2.0 | 1880.8 | 4.8 | 391.8 |
| YDE011 1 uM | 3rd | 5630.0 | 2.0 | 1714.5 | 4.8 | 357.2 |
| | Mean | 5970.2 | 2.0 | 1797.0 | 5.0 | 358.5 |
| | STDER | 197.8 | 0.0 | 48.0 | 0.2 | 18.9 |
| | 1st | 4743.0 | 2.0 | 1502.6 | 5.4 | 278.3 |
| PBMC + | 2nd | 5215.5 | 2.0 | 1615.2 | 5.8 | 278.5 |
| LPS(5 ug/ml) + | 3rd | 5049.0 | 2.0 | 1575.4 | 4.7 | 335.2 |
| YDE011 | Mean | 5002.5 | 2.0 | 1564.4 | 5.3 | 297.3 |
| 10 uM | STDER | 138.4 | 0.0 | 33.0 | 0.3 | 18.9 |
| PBMC + | 1st | 262.0 | 2.0 | 252.0 | 6.1 | 41.3 |
| LPS(5 ug/ml) + | 2nd | 360.0 | 2.0 | 301.1 | 6.1 | 49.4 |
| YDE011 | 3rd | 342.0 | 2.0 | 292.6 | 6.0 | 48.8 |
| 100 uM | Mean | 321.3 | 2.0 | 281.9 | 6.1 | 46.5 |
| | STDER | 30.1 | 0.0 | 15.2 | 0.0 | 2.6 |
| PBMC + | 1st | 7127.0 | 2.0 | 2082.8 | 4.4 | 473.4 |
| LPS(5 ug/ml) + | 2nd | 7646.5 | 2.0 | 2215.5 | 6.0 | 369.2 |
| YDE043 | 3rd | 6916.0 | 2.0 | 2029.7 | 4.6 | 441.2 |
| 0.001 uM | Mean | 7229.8 | 2.0 | 2109.3 | 5.0 | 427.9 |
| | STDER | 217.1 | 0.0 | 55.2 | 0.5 | 30.8 |
| PBMC + | 1st | 6942.0 | 2.0 | 2036.2 | 4.5 | 452.5 |
| LPS(5 ug/ml) + | 2nd | 6650.0 | 2.0 | 1963.4 | 6.6 | 297.5 |
| YDE043 | 3rd | 7082.0 | 2.0 | 2071.4 | 5.0 | 414.3 |
| 0.01 uM | Mean | 6891.3 | 2.0 | 2023.7 | 5.4 | 388.1 |
| | STDER | 127.3 | 0.0 | 31.8 | 0.6 | 46.6 |
| PBMC + | 1st | 7234.0 | 2.0 | 2109.9 | 5.0 | 422.0 |
| LPS(5 ug/ml) + | 2nd | 5852.5 | 2.0 | 1768.2 | 5.6 | 315.8 |
| YDE043 | 3rd | 6350.5 | 2.0 | 1889.5 | 5.6 | 337.4 |
| 0.1 uM | Mean | 6479.0 | 2.0 | 1922.5 | 5.4 | 358.4 |
| | STDER | 403.9 | 0.0 | 100.0 | 0.2 | 32.4 |
| PBMC + | 1st | 7486.0 | 2.0 | 2174.1 | 5.8 | 374.9 |
| LPS(5 ug/ml) + | 2nd | 7253.0 | 2.0 | 2114.7 | 4.8 | 440.6 |
| YDE043 1 uM | 3rd | 7122.0 | 2.0 | 2081.5 | 5.3 | 392.7 |
| | Mean | 7287.0 | 2.0 | 2123.4 | 5.3 | 402.7 |
| | STDER | 106.4 | 0.0 | 27.1 | 0.3 | 19.6 |
| PBMC + | 1st | 6348.5 | 2.0 | 1889.0 | 5.2 | 363.3 |
| LPS(5 ug/ml) + | 2nd | 5845.0 | 2.0 | 1766.4 | 5.4 | 327.1 |
| YDE043 | 3rd | 5753.0 | 2.0 | 1744.2 | 5.6 | 311.5 |
| 10 uM | Mean | 5982.2 | 2.0 | 1799.9 | 5.4 | 333.9 |
| | STDER | 185.1 | 0.0 | 45.0 | 0.1 | 15.3 |
| PBMC + | 1st | 275.0 | 2.0 | 258.9 | 6.2 | 41.8 |
| LPS(5 ug/ml) + | 2nd | 364.0 | 2.0 | 303.0 | 6.4 | 47.3 |
| YDE043 | Mean | 319.5 | 2.0 | 281.0 | 6.3 | 44.6 |
| 100 uM | STDER | 44.5 | 0.0 | 22.1 | 0.1 | 2.8 |

TABLE 36
Bead name: Fas Ligand

| | | | | Con- | Live | conc/ |
|---|---|---|---|---|---|---|
| | | | | cen- | cell | live |
| | | | Dilu- | tration | (10\hat{ }5/ | cell (pg/ |
| Sample | | MFI | tion | (pg/ml) | ml) | 10\hat{ }5) |
| Blank | 1st | 0.0 | 2.0 | NaN | | |
| | 2nd | 0.0 | 2.0 | NaN | | |
| | Mean | 0.0 | 2.0 | #DIV/0! | #DIV/0! | #DIV/0! |
| | STDER | 0.0 | 0.0 | #DIV/0! | #DIV/0! | #DIV/0! |
| PBMC + | 1st | 4.0 | 2.0 | 4.6 | 7.5 | 0.6 |
| 0.5% DMSO | 2nd | 4.0 | 2.0 | 4.6 | 7.4 | 0.6 |
| | 3rd | 4.0 | 2.0 | 4.6 | 6.6 | 0.7 |
| | Mean | 4.0 | 2.0 | 4.6 | 7.2 | 0.6 |
| | STDER | 0.0 | 0.0 | 0.0 | 0.3 | 0.0 |
| PBMC + | 1st | 32.0 | 2.0 | 23.1 | 6.1 | 3.8 |
| LPS(5 ug/ml) | 2nd | 47.0 | 2.0 | 33.4 | 5.8 | 5.8 |
| | 3rd | 45.0 | 2.0 | 32.0 | 5.5 | 5.8 |
| | Mean | 41.3 | 2.0 | 29.5 | 5.8 | 5.1 |
| | STDER | 4.7 | 0.0 | 3.2 | 0.2 | 0.7 |
| PBMC + YY101 | 1st | 7.0 | 2.0 | 6.5 | 5.2 | 1.3 |
| 0.01 uM | 2nd | 10.5 | 2.0 | 8.8 | 4.8 | 1.8 |
| | Mean | 8.8 | 2.0 | 7.7 | 5.0 | 1.5 |
| | STDER | 1.8 | 0.0 | 1.1 | 0.2 | 0.3 |
| PBMC + YY101 | 1st | 7.0 | 2.0 | 6.5 | 4.4 | 1.5 |
| 0.1 uM | 2nd | 9.0 | 2.0 | 7.8 | 5.8 | 1.3 |
| | Mean | 8.0 | 2.0 | 7.2 | 5.1 | 1.4 |
| | STDER | 1.0 | 0.0 | 0.6 | 0.7 | 0.1 |
| PBMC + YY101 | 1st | 7.0 | 2.0 | 6.5 | 5.5 | 1.2 |
| 1 uM | 2nd | 10.0 | 2.0 | 8.5 | 5.5 | 1.5 |
| | Mean | 8.5 | 2.0 | 7.5 | 5.5 | 1.4 |
| | STDER | 1.5 | 0.0 | 1.0 | 0.0 | 0.2 |
| PBMC + YY101 | 1st | 18.5 | 2.0 | 14.0 | 6.4 | 2.2 |
| 10 uM | 2nd | 16.0 | 2.0 | 12.4 | 6.1 | 2.0 |
| | Mean | 17.3 | 2.0 | 13.2 | 6.3 | 2.1 |
| | STDER | 1.3 | 0.0 | 0.8 | 0.2 | 0.1 |
| PBMC + YY101 | 1st | 9.0 | 2.0 | 7.8 | 6.4 | 1.2 |
| 100 uM | 2nd | 6.0 | 2.0 | 5.9 | 7.0 | 0.8 |
| | Mean | 7.5 | 2.0 | 6.9 | 6.7 | 1.0 |
| | STDER | 1.5 | 0.0 | 1.0 | 0.3 | 0.2 |
| PBMC + | 1st | 77.0 | 2.0 | 54.4 | 6.2 | 8.8 |
| LPS(5 ug/ml) + | 2nd | 45.0 | 2.0 | 32.0 | 7.6 | 4.2 |
| Xiidra 0.001 uM | Mean | 61.0 | 2.0 | 43.2 | 6.9 | 6.5 |
| | STDER | 16.0 | 0.0 | 11.2 | 0.7 | 2.3 |
| PBMC + | 1st | 56.0 | 2.0 | 39.7 | 5.2 | 7.6 |
| LPS(5 ug/ml) + | 2nd | 41.0 | 2.0 | 29.3 | 5.8 | 5.0 |
| Xiidra 0.01 uM | Mean | 48.5 | 2.0 | 34.5 | 5.5 | 6.3 |
| | STDER | 7.5 | 0.0 | 5.2 | 0.3 | 1.3 |
| PBMC + | 1st | 36.5 | 2.0 | 26.2 | 6.6 | 4.0 |
| LPS(5 ug/ml) + | 2nd | 37.5 | 2.0 | 26.9 | 5.7 | 4.7 |
| Xiidra 0.1 uM | Mean | 37.0 | 2.0 | 26.5 | 6.2 | 4.3 |
| | STDER | 0.5 | 0.0 | 0.3 | 0.5 | 0.4 |
| PBMC + | 1st | 37.0 | 2.0 | 26.5 | 4.8 | 5.5 |
| LPS(5 ug/ml) + | 2nd | 24.5 | 2.0 | 18.1 | 4.4 | 4.1 |
| Xiidra 1 uM | Mean | 30.8 | 2.0 | 22.3 | 4.6 | 4.8 |
| | STDER | 6.3 | 0.0 | 4.2 | 0.2 | 0.7 |
| PBMC + | 1st | 34.0 | 2.0 | 24.5 | 5.8 | 4.2 |
| LPS(5 ug/ml) + | 2nd | 44.5 | 2.0 | 31.7 | 6.4 | 5.0 |
| Xiidra 10 uM | Mean | 39.3 | 2.0 | 28.1 | 6.1 | 4.6 |
| | STDER | 5.3 | 0.0 | 3.6 | 0.3 | 0.4 |
| PBMC + | 1st | 42.0 | 2.0 | 30.0 | 7.1 | 4.2 |
| LPS(5 ug/ml) + | 2nd | 40.0 | 2.0 | 28.6 | 5.8 | 4.9 |
| Xiidra 100 uM | Mean | 41.0 | 2.0 | 29.3 | 6.5 | 4.6 |
| | STDER | 1.0 | 0.0 | 0.7 | 0.7 | 0.4 |
| PBMC + | 1st | 50.5 | 2.0 | 35.8 | 5.7 | 6.3 |
| LPS(5 ug/ml) + | 2nd | 46.0 | 2.0 | 32.7 | 7.1 | 4.6 |
| YY101 | 3rd | 33.5 | 2.0 | 24.1 | 6.6 | 3.7 |
| 0.001 uM | Mean | 43.3 | 2.0 | 30.9 | 6.5 | 4.9 |
| | STDER | 5.1 | 0.0 | 3.5 | 0.4 | 0.8 |
| PBMC + | 1st | 43.0 | 2.0 | 30.7 | 6.4 | 4.8 |
| LPS(5 ug/ml) + | 2nd | 55.5 | 2.0 | 39.3 | 6.0 | 6.6 |
| YY101 0.01 uM | 3rd | 44.0 | 2.0 | 31.3 | 6.1 | 5.1 |
| | Mean | 47.5 | 2.0 | 33.8 | 6.2 | 5.5 |
| | STDER | 4.0 | 0.0 | 2.8 | 0.1 | 0.5 |
| PBMC + | 1st | 46.0 | 2.0 | 32.7 | 6.2 | 5.3 |
| LPS(5 ug/ml) + | 2nd | 43.0 | 2.0 | 30.7 | 5.2 | 5.9 |
| YY101 0.1 uM | 3rd | 35.0 | 2.0 | 25.2 | 6.2 | 4.1 |
| | Mean | 41.3 | 2.0 | 29.5 | 5.9 | 5.1 |
| | STDER | 3.3 | 0.0 | 2.3 | 0.3 | 0.5 |
| PBMC + | 1st | 50.0 | 2.0 | 35.5 | 6.3 | 5.6 |
| LPS(5 ug/ml) + | 2nd | 51.0 | 2.0 | 36.2 | 7.1 | 5.1 |
| YY101 1 uM | 3rd | 44.5 | 2.0 | 31.7 | 6.2 | 5.1 |
| | Mean | 48.5 | 2.0 | 34.5 | 6.5 | 5.3 |
| | STDER | 2.0 | 0.0 | 1.4 | 0.3 | 0.2 |
| PBMC + | 1st | 82.0 | 2.0 | 58.0 | 6.0 | 9.7 |
| LPS(5 ug/ml) + | 2nd | 76.0 | 2.0 | 53.7 | 6.4 | 8.4 |
| YY101 10 uM | 3rd | 40.0 | 2.0 | 28.6 | 7.6 | 3.8 |
| | Mean | 66.0 | 2.0 | 46.7 | 6.7 | 7.3 |
| | STDER | 13.1 | 0.0 | 9.2 | 0.5 | 1.8 |
| PBMC + | 1st | 55.0 | 2.0 | 39.0 | 6.3 | 6.2 |
| LPS(5 ug/ml) + | 2nd | 21.5 | 2.0 | 16.0 | 6.2 | 2.6 |
| YY101 100 uM | 3rd | 29.0 | 2.0 | 21.1 | 5.0 | 4.2 |
| | Mean | 35.2 | 2.0 | 25.4 | 5.8 | 4.3 |
| | STDER | 10.2 | 0.0 | 7.0 | 0.4 | 1.0 |
| PBMC + | 1st | 21.0 | 2.0 | 15.7 | 4.5 | 3.5 |
| LPS(5 ug/ml) + | 2nd | 25.0 | 2.0 | 18.4 | 5.8 | 3.2 |
| YDE011 0.001 | 3rd | 22.0 | 2.0 | 16.4 | 5.8 | 2.8 |
| uM | Mean | 22.7 | 2.0 | 16.8 | 5.4 | 3.2 |
| | STDER | 1.2 | 0.0 | 0.8 | 0.4 | 0.2 |
| PBMC + | 1st | 23.0 | 2.0 | 17.1 | 6.3 | 2.7 |
| LPS(5 ug/ml) + | 2nd | 18.0 | 2.0 | 13.7 | 6.2 | 2.2 |
| YDE011 | 3rd | 21.0 | 2.0 | 15.7 | 5.1 | 3.1 |
| 0.01 uM | Mean | 20.7 | 2.0 | 15.5 | 5.9 | 2.7 |
| | STDER | 1.5 | 0.0 | 1.0 | 0.4 | 0.3 |
| PBMC + | 1st | 26.0 | 2.0 | 19.1 | 6.6 | 2.9 |
| LPS(5 ug/ml) + | 2nd | 24.0 | 2.0 | 17.7 | 5.0 | 3.5 |
| YDE011 0.1 uM | 3rd | 20.0 | 2.0 | 15.0 | 5.7 | 2.6 |
| | Mean | 23.3 | 2.0 | 17.3 | 5.8 | 3.0 |
| | STDER | 1.8 | 0.0 | 1.2 | 0.5 | 0.3 |
| PBMC + | 1st | 23.5 | 2.0 | 17.4 | 5.5 | 3.2 |
| LPS(5 ug/ml) + | 2nd | 20.5 | 2.0 | 15.4 | 4.8 | 3.2 |
| YDE011 1 uM | 3rd | 20.0 | 2.0 | 15.0 | 4.8 | 3.1 |
| | Mean | 21.3 | 2.0 | 15.9 | 5.0 | 3.2 |
| | STDER | 1.1 | 0.0 | 0.7 | 0.2 | 0.0 |
| PBMC + | 1st | 27.0 | 2.0 | 19.7 | 5.4 | 3.7 |
| LPS(5 ug/ml) + | 2nd | 24.0 | 2.0 | 17.7 | 5.8 | 3.1 |
| YDE011 10 uM | 3rd | 19.0 | 2.0 | 14.4 | 4.7 | 3.1 |
| | Mean | 23.3 | 2.0 | 17.3 | 5.3 | 3.3 |
| | STDER | 2.3 | 0.0 | 1.6 | 0.3 | 0.2 |
| PBMC + | 1st | 15.0 | 2.0 | 11.7 | 6.1 | 1.9 |
| LPS(5 ug/ml) + | 2nd | 17.0 | 2.0 | 13.1 | 6.1 | 2.1 |
| YDE011 | 3rd | 20.0 | 2.0 | 15.0 | 6.0 | 2.5 |
| 100 uM | Mean | 17.3 | 2.0 | 13.3 | 6.1 | 2.2 |
| | STDER | 1.5 | 0.0 | 1.0 | 0.0 | 0.2 |
| PBMC + | 1st | 23.0 | 2.0 | 17.1 | 4.4 | 3.9 |
| LPS(5 ug/ml) + | 2nd | 28.0 | 2.0 | 20.4 | 6.0 | 3.4 |
| YDE043 | 3rd | 17.0 | 2.0 | 13.1 | 4.6 | 2.8 |
| 0.001 uM | Mean | 22.7 | 2.0 | 16.8 | 5.0 | 3.4 |
| | STDER | 3.2 | 0.0 | 2.1 | 0.5 | 0.3 |
| PBMC + | 1st | 26.0 | 2.0 | 19.1 | 4.5 | 4.2 |
| LPS(5 ug/ml) + | 2nd | 21.0 | 2.0 | 15.7 | 6.6 | 2.4 |
| YDE043 | 3rd | 43.5 | 2.0 | 31.0 | 5.0 | 6.2 |
| 0.01 uM | Mean | 30.2 | 2.0 | 21.9 | 5.4 | 4.3 |
| | STDER | 6.8 | 0.0 | 4.6 | 0.6 | 1.1 |
| PBMC + | 1st | 19.0 | 2.0 | 14.4 | 5.0 | 2.9 |
| LPS(5 ug/ml) + | 2nd | 16.5 | 2.0 | 12.7 | 5.6 | 2.3 |
| YDE043 0.1 uM | 3rd | 16.0 | 2.0 | 12.4 | 5.6 | 2.2 |
| | Mean | 17.2 | 2.0 | 13.2 | 5.4 | 2.5 |
| | STDER | 0.9 | 0.0 | 0.6 | 0.2 | 0.2 |
| PBMC + | 1st | 18.0 | 2.0 | 13.7 | 5.8 | 2.4 |
| LPS(5 ug/ml) + | 2nd | 21.0 | 2.0 | 15.7 | 4.8 | 3.3 |
| YDE043 1 uM | 3rd | 28.0 | 2.0 | 20.4 | 5.3 | 3.9 |
| | Mean | 22.3 | 2.0 | 16.6 | 5.3 | 3.2 |
| | STDER | 3.0 | 0.0 | 2.0 | 0.3 | 0.4 |
| PBMC + | 1st | 22.0 | 2.0 | 16.4 | 5.2 | 3.2 |
| LPS(5 ug/ml) + | 2nd | 23.0 | 2.0 | 17.1 | 5.4 | 3.2 |
| YDE043 10 uM | 3rd | 21.0 | 2.0 | 15.7 | 5.6 | 2.8 |
| | Mean | 22.0 | 2.0 | 16.4 | 5.4 | 3.0 |
| | STDER | 0.6 | 0.0 | 0.4 | 0.1 | 0.1 |
| PBMC + | 1st | 20.0 | 2.0 | 15.0 | 6.2 | 2.4 |
| LPS(5 ug/ml) + | 2nd | 14.5 | 2.0 | 11.4 | 6.4 | 1.8 |
| YDE043 | Mean | 17.3 | 2.0 | 13.2 | 6.3 | 2.1 |
| 100 uM | STDER | 2.8 | 0.0 | 1.8 | 0.1 | 0.3 |

TABLE 37
Bead name: CCL20/MIP-3 alpha

| | | | | Con- | Live | conc/ |
|---|---|---|---|---|---|---|
| | | | | cen- | cell | live |
| | | | Dilu- | tration | (10\hat{ }5/ | cell (pg/ |
| Sample | | MFI | tion | (pg/ml) | ml) | 10\hat{ }5) |
| Blank | 1st | 0.5 | 2.0 | NaN | | |
| | 2nd | −0.5 | 2.0 | NaN | | |
| | Mean | 0.0 | 2.0 | #DIV/0! | #DIV/0! | #DIV/0! |
| | STDER | 0.5 | 0.0 | #DIV/0! | #DIV/0! | #DIV/0! |
| PBMC + | 1st | 3.5 | 2.0 | | 7.5 | 0.0 |
| 0.5% DMSO | 2nd | 2.0 | 2.0 | | 7.4 | 0.0 |
| | 3rd | 3.5 | 2.0 | | 6.6 | 0.0 |
| | Mean | 3.0 | 2.0 | #DIV/0! | 7.2 | 0.0 |
| | STDER | 0.5 | 0.0 | #DIV/0! | 0.3 | 0.0 |
| PBMC + | 1st | 432.0 | 2.0 | 384.5 | 6.1 | 63.0 |
| LPS(5 ug/ml) | 2nd | 390.5 | 2.0 | 351.1 | 5.8 | 60.5 |
| | 3rd | 428.5 | 2.0 | 381.7 | 5.5 | 69.4 |
| | Mean | 417.0 | 2.0 | 372.4 | 5.8 | 64.3 |
| | STDER | 13.3 | 0.0 | 10.7 | 0.2 | 2.6 |
| PBMC + YY101 | 1st | 7.5 | 2.0 | 7.3 | 5.2 | 1.4 |
| 0.01 uM | 2nd | 14.5 | 2.0 | 16.8 | 4.8 | 3.5 |
| | Mean | 11.0 | 2.0 | 12.1 | 5.0 | 2.5 |
| | STDER | 3.5 | 0.0 | 4.7 | 0.2 | 1.0 |
| PBMC + | 1st | 7.5 | 2.0 | 7.3 | 4.4 | 1.7 |
| YY101 0.1 uM | 2nd | 10.5 | 2.0 | 11.6 | 5.8 | 2.0 |
| | Mean | 9.0 | 2.0 | 9.5 | 5.1 | 1.8 |
| | STDER | 1.5 | 0.0 | 2.1 | 0.7 | 0.2 |
| PBMC + | 1st | 7.5 | 2.0 | 7.3 | 5.5 | 1.3 |
| YY101 1 uM | 2nd | 9.5 | 2.0 | 10.2 | 5.5 | 1.9 |
| | Mean | 8.5 | 2.0 | 8.8 | 5.5 | 1.6 |
| | STDER | 1.0 | 0.0 | 1.4 | 0.0 | 0.3 |
| PBMC + | 1st | 51.5 | 2.0 | 57.4 | 6.4 | 9.0 |
| YY101 10 uM | 2nd | 21.5 | 2.0 | 25.3 | 6.1 | 4.1 |
| | Mean | 36.5 | 2.0 | 41.3 | 6.3 | 6.6 |
| | STDER | 15.0 | 0.0 | 16.1 | 0.2 | 2.4 |
| PBMC + | 1st | 25.5 | 2.0 | 29.8 | 6.4 | 4.7 |
| YY101 100 uM | 2nd | 13.0 | 2.0 | 14.9 | 7.0 | 2.1 |
| | Mean | 19.3 | 2.0 | 22.4 | 6.7 | 3.4 |
| | STDER | 6.3 | 0.0 | 7.5 | 0.3 | 1.3 |
| PBMC + | 1st | 453.5 | 2.0 | 401.8 | 6.2 | 64.8 |
| LPS(5 ug/ml) + | 2nd | 392.5 | 2.0 | 352.7 | 7.6 | 46.4 |
| Xiidra 0.001 uM | Mean | 423.0 | 2.0 | 377.2 | 6.9 | 55.6 |
| | STDER | 30.5 | 0.0 | 24.5 | 0.7 | 9.2 |
| PBMC + | 1st | 485.5 | 2.0 | 427.3 | 5.2 | 82.2 |
| LPS(5 ug/ml) + | 2nd | 377.5 | 2.0 | 340.5 | 5.8 | 58.7 |
| Xiidra 0.01 uM | Mean | 431.5 | 2.0 | 383.9 | 5.5 | 70.4 |
| | STDER | 54.0 | 0.0 | 43.4 | 0.3 | 11.7 |
| PBMC + | 1st | 306.5 | 2.0 | 282.4 | 6.6 | 42.8 |
| LPS(5 ug/ml) + | 2nd | 248.5 | 2.0 | 234.1 | 5.7 | 41.1 |
| Xiidra 0.1 uM | Mean | 277.5 | 2.0 | 258.3 | 6.2 | 41.9 |
| | STDER | 29.0 | 0.0 | 24.2 | 0.5 | 0.9 |
| PBMC + | 1st | 459.5 | 2.0 | 406.6 | 4.8 | 84.7 |
| LPS(5 ug/ml) + | 2nd | 258.5 | 2.0 | 242.5 | 4.4 | 55.1 |
| Xiidra 1 uM | Mean | 359.0 | 2.0 | 324.5 | 4.6 | 69.9 |
| | STDER | 100.5 | 0.0 | 82.0 | 0.2 | 14.8 |
| PBMC + | 1st | 356.5 | 2.0 | 323.4 | 5.8 | 55.8 |
| LPS(5 ug/ml) + | 2nd | 353.0 | 2.0 | 320.6 | 6.4 | 50.1 |
| Xiidra 10 uM | Mean | 354.8 | 2.0 | 322.0 | 6.1 | 52.9 |
| | STDER | 1.8 | 0.0 | 1.4 | 0.3 | 2.8 |
| PBMC + | 1st | 228.5 | 2.0 | 217.2 | 7.1 | 30.6 |
| LPS(5 ug/ml) + | 2nd | 253.0 | 2.0 | 237.9 | 5.8 | 41.0 |
| Xiidra 100 uM | Mean | 240.8 | 2.0 | 227.6 | 6.5 | 35.8 |
| | STDER | 12.3 | 0.0 | 10.3 | 0.7 | 5.2 |
| PBMC + | 1st | 329.0 | 2.0 | 301.0 | 5.7 | 52.8 |
| LPS(5 ug/ml) + | 2nd | 315.5 | 2.0 | 289.9 | 7.1 | 40.8 |
| YY101 | 3rd | 395.5 | 2.0 | 355.1 | 6.6 | 53.8 |
| 0.001 uM | Mean | 346.7 | 2.0 | 315.3 | 6.5 | 49.1 |
| | STDER | 24.7 | 0.0 | 20.2 | 0.4 | 4.2 |
| PBMC + | 1st | 431.0 | 2.0 | 383.7 | 6.4 | 60.0 |
| LPS(5 ug/ml) + | 2nd | 375.5 | 2.0 | 338.9 | 6.0 | 56.5 |
| YY101 0.01 uM | 3rd | 464.5 | 2.0 | 410.6 | 6.1 | 67.3 |
| | Mean | 423.7 | 2.0 | 377.7 | 6.2 | 61.2 |
| | STDER | 26.0 | 0.0 | 20.9 | 0.1 | 3.2 |
| PBMC + | 1st | 424.5 | 2.0 | 378.5 | 6.2 | 61.0 |
| LPS(5 ug/ml) + | 2nd | 407.0 | 2.0 | 364.4 | 5.2 | 70.1 |
| YY101 0.1 uM | 3rd | 388.5 | 2.0 | 349.4 | 6.2 | 56.4 |
| | Mean | 406.7 | 2.0 | 364.1 | 5.9 | 62.5 |
| | STDER | 10.4 | 0.0 | 8.4 | 0.3 | 4.0 |
| PBMC + | 1st | 356.0 | 2.0 | 323.0 | 6.3 | 51.3 |
| LPS(5 ug/ml) + | 2nd | 364.5 | 2.0 | 330.0 | 7.1 | 46.5 |
| YY101 1 uM | 3rd | 404.5 | 2.0 | 362.4 | 6.2 | 58.4 |
| | Mean | 375.0 | 2.0 | 338.5 | 6.5 | 52.1 |
| | STDER | 15.0 | 0.0 | 12.1 | 0.3 | 3.5 |
| PBMC + | 1st | 357.5 | 2.0 | 324.3 | 6.0 | 54.0 |
| LPS(5 ug/ml) + | 2nd | 423.5 | 2.0 | 377.7 | 6.4 | 59.0 |
| YY101 10 uM | 3rd | 454.5 | 2.0 | 402.6 | 7.6 | 53.0 |
| | Mean | 411.8 | 2.0 | 368.2 | 6.7 | 55.3 |
| | STDER | 28.6 | 0.0 | 23.1 | 0.5 | 1.9 |
| PBMC + | 1st | 692.5 | 2.0 | 590.1 | 6.3 | 93.7 |
| LPS(5 ug/ml) + | 2nd | 694.5 | 2.0 | 591.7 | 6.2 | 95.4 |
| YY101 100 uM | 3rd | 646.0 | 2.0 | 553.9 | 5.0 | 110.8 |
| | Mean | 677.7 | 2.0 | 578.6 | 5.8 | 100.0 |
| | STDER | 15.8 | 0.0 | 12.4 | 0.4 | 5.4 |
| PBMC + | 1st | 284.5 | 2.0 | 264.2 | 4.5 | 58.7 |
| LPS(5 ug/ml) + | 2nd | 235.5 | 2.0 | 223.2 | 5.8 | 38.5 |
| YDE011 | 3rd | 270.0 | 2.0 | 252.1 | 5.8 | 43.5 |
| 0.001 uM | Mean | 263.3 | 2.0 | 246.5 | 5.4 | 46.9 |
| | STDER | 14.5 | 0.0 | 12.2 | 0.4 | 6.1 |
| PBMC + | 1st | 247.5 | 2.0 | 233.3 | 6.3 | 37.0 |
| LPS(5 ug/ml) + | 2nd | 278.0 | 2.0 | 258.8 | 6.2 | 41.7 |
| YDE011 | 3rd | 297.0 | 2.0 | 274.6 | 5.1 | 53.8 |
| 0.01 uM | Mean | 274.2 | 2.0 | 255.6 | 5.9 | 44.2 |
| | STDER | 14.4 | 0.0 | 12.0 | 0.4 | 5.0 |
| PBMC + | 1st | 253.5 | 2.0 | 238.3 | 6.6 | 36.1 |
| LPS(5 ug/ml) + | 2nd | 245.5 | 2.0 | 231.6 | 5.0 | 46.3 |
| YDE011 0.1 uM | 3rd | 265.5 | 2.0 | 248.4 | 5.7 | 43.6 |
| | Mean | 254.8 | 2.0 | 239.4 | 5.8 | 42.0 |
| | STDER | 5.8 | 0.0 | 4.9 | 0.5 | 3.1 |
| PBMC + | 1st | 212.5 | 2.0 | 203.6 | 5.5 | 37.0 |
| LPS(5 ug/ml) + | 2nd | 218.5 | 2.0 | 208.7 | 4.8 | 43.5 |
| YDE011 1 uM | 3rd | 233.5 | 2.0 | 221.5 | 4.8 | 46.1 |
| | Mean | 221.5 | 2.0 | 211.3 | 5.0 | 42.2 |
| | STDER | 6.2 | 0.0 | 5.3 | 0.2 | 2.7 |
| PBMC + | 1st | 248.5 | 2.0 | 234.1 | 5.4 | 43.4 |
| LPS(5 ug/ml) + | 2nd | 234.5 | 2.0 | 222.3 | 5.8 | 38.3 |
| YDE011 10 uM | 3rd | 249.0 | 2.0 | 234.5 | 4.7 | 49.9 |
| | Mean | 244.0 | 2.0 | 230.3 | 5.3 | 43.9 |
| | STDER | 4.8 | 0.0 | 4.0 | 0.3 | 3.4 |
| PBMC + | 1st | 379.5 | 2.0 | 342.2 | 6.1 | 56.1 |
| LPS(5 ug/ml) + | 2nd | 351.5 | 2.0 | 319.4 | 6.1 | 52.4 |
| YDE011 | 3rd | 338.5 | 2.0 | 308.7 | 6.0 | 51.5 |
| 100 uM | Mean | 356.5 | 2.0 | 323.4 | 6.1 | 53.3 |
| | STDER | 12.1 | 0.0 | 9.9 | 0.0 | 1.4 |
| PBMC + | 1st | 252.5 | 2.0 | 237.5 | 4.4 | 54.0 |
| LPS(5 ug/ml) + | 2nd | 225.0 | 2.0 | 214.3 | 6.0 | 35.7 |
| YDE043 | 3rd | 225.5 | 2.0 | 214.7 | 4.6 | 46.7 |
| 0.001 uM | Mean | 234.3 | 2.0 | 222.1 | 5.0 | 45.5 |
| | STDER | 9.1 | 0.0 | 7.7 | 0.5 | 5.3 |
| PBMC + | 1st | 294.0 | 2.0 | 272.1 | 4.5 | 60.5 |
| LPS(5 ug/ml) + | 2nd | 250.5 | 2.0 | 235.8 | 6.6 | 35.7 |
| YDE043 | 3rd | 284.5 | 2.0 | 264.2 | 5.0 | 52.8 |
| 0.01 uM | Mean | 276.3 | 2.0 | 257.4 | 5.4 | 49.7 |
| | STDER | 13.2 | 0.0 | 11.0 | 0.6 | 7.3 |
| PBMC + | 1st | 291.0 | 2.0 | 269.6 | 5.0 | 53.9 |
| LPS(5 ug/ml) + | 2nd | 287.5 | 2.0 | 266.7 | 5.6 | 47.6 |
| YDE043 0.1 uM | 3rd | 294.0 | 2.0 | 272.1 | 5.6 | 48.6 |
| | Mean | 290.8 | 2.0 | 269.5 | 5.4 | 50.0 |
| | STDER | 1.9 | 0.0 | 1.6 | 0.2 | 2.0 |
| PBMC + | 1st | 288.0 | 2.0 | 267.1 | 5.8 | 46.1 |
| LPS(5 ug/ml) + | 2nd | 269.5 | 2.0 | 251.7 | 4.8 | 52.4 |
| YDE043 1 uM | 3rd | 272.5 | 2.0 | 254.2 | 5.3 | 48.0 |
| | Mean | 276.7 | 2.0 | 257.7 | 5.3 | 48.8 |
| | STDER | 5.7 | 0.0 | 4.8 | 0.3 | 1.9 |
| PBMC + | 1st | 279.5 | 2.0 | 260.1 | 5.2 | 50.0 |
| LPS(5 ug/ml) + | 2nd | 280.5 | 2.0 | 260.9 | 5.4 | 48.3 |
| YDE043 10 uM | 3rd | 306.5 | 2.0 | 282.4 | 5.6 | 50.4 |
| | Mean | 288.8 | 2.0 | 267.8 | 5.4 | 49.6 |
| | STDER | 8.8 | 0.0 | 7.3 | 0.1 | 0.6 |
| PBMC + | 1st | 428.5 | 2.0 | 381.7 | 6.2 | 61.6 |
| LPS(5 ug/ml) + | 2nd | 370.5 | 2.0 | 334.8 | 6.4 | 52.3 |
| YDE043 | Mean | 399.5 | 2.0 | 358.3 | 6.3 | 56.9 |
| 100 uM | STDER | 29.0 | 0.0 | 23.4 | 0.1 | 4.6 |

TABLE 38
Bead name: IL-1 alpha

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.5 | 2.0 | NaN | | |
| | 2nd | −0.5 | 2.0 | NaN | | |
| | Mean | 0.0 | 2.0 | #DIV/0! | #DIV/0! | #DIV/0! |
| | STDER | 0.5 | 0.0 | #DIV/0! | #DIV/0! | #DIV/0! |
| PBMC + 0.5% DMSO | 1st | 0.0 | 2.0 | 3.3 | 7.5 | 0.4 |
| | 2nd | −1.0 | 2.0 | 1.0 | 7.4 | 0.1 |
| | 3rd | −1.0 | 2.0 | 1.0 | 6.6 | 0.1 |
| | Mean | −0.7 | 2.0 | 1.8 | 7.2 | 0.2 |
| | STDER | 0.3 | 0.0 | 0.8 | 0.3 | 0.1 |
| PBMC + LPS(5 ug/ml) | 1st | 516.5 | 2.0 | 435.0 | 6.1 | 71.3 |
| | 2nd | 483.0 | 2.0 | 410.3 | 5.8 | 70.7 |
| | 3rd | 466.5 | 2.0 | 398.1 | 5.5 | 72.4 |
| | Mean | 488.7 | 2.0 | 414.5 | 5.8 | 71.5 |
| | STDER | 14.7 | 0.0 | 10.9 | 0.2 | 0.5 |
| PBMC + YY101 0.01 uM | 1st | −1.0 | 2.0 | 1.0 | 5.2 | 0.2 |
| | 2nd | 1.0 | 2.0 | 5.3 | 4.8 | 1.1 |
| | Mean | 0.0 | 2.0 | 3.1 | 5.0 | 0.6 |
| | STDER | 1.0 | 0.0 | 2.2 | 0.2 | 0.5 |
| PBMC + YY101 0.01 uM | 1st | −1.0 | 2.0 | 1.0 | 4.4 | 0.2 |
| | 2nd | 1.0 | 2.0 | 5.3 | 5.8 | 0.9 |
| | Mean | 0.0 | 2.0 | 3.1 | 5.1 | 0.6 |
| | STDER | 1.0 | 0.0 | 2.2 | 0.7 | 0.3 |
| PBMC + YY101 1 uM | 1st | 1.0 | 2.0 | 5.3 | 5.5 | 1.0 |
| | 2nd | 1.0 | 2.0 | 5.3 | 5.5 | 1.0 |
| | Mean | 1.0 | 2.0 | 5.3 | 5.5 | 1.0 |
| | STDER | 0.0 | 0.0 | 0.0 | 0.0 | 0.0 |
| PBMC + YY101 10 uM | 1st | 20.0 | 2.0 | 31.7 | 6.4 | 5.0 |
| | 2nd | 2.0 | 2.0 | 7.1 | 6.1 | 1.2 |
| | Mean | 11.0 | 2.0 | 19.4 | 6.3 | 3.1 |
| | STDER | 9.0 | 0.0 | 12.3 | 0.2 | 1.9 |
| PBMC + YY101 100 uM | 1st | 4.0 | 2.0 | 10.4 | 6.4 | 1.6 |
| | 2nd | 1.0 | 2.0 | 5.3 | 7.0 | 0.8 |
| | Mean | 2.5 | 2.0 | 7.9 | 6.7 | 1.2 |
| | STDER | 1.5 | 0.0 | 2.5 | 0.3 | 0.4 |
| PBMC + LPS(5 ug/ml) + Xiidra 0.001 uM | 1st | 599.0 | 2.0 | 495.8 | 6.2 | 80.0 |
| | 2nd | 545.0 | 2.0 | 456.0 | 7.6 | 60.0 |
| | Mean | 572.0 | 2.0 | 475.9 | 6.9 | 70.0 |
| | STDER | 27.0 | 0.0 | 19.9 | 0.7 | 10.0 |
| PBMC + LPS(5 ug/ml) + Xiidra 0.01 uM | 1st | 651.0 | 2.0 | 534.1 | 5.2 | 102.7 |
| | 2nd | 539.0 | 2.0 | 451.6 | 5.8 | 77.9 |
| | Mean | 595.0 | 2.0 | 492.8 | 5.5 | 90.3 |
| | STDER | 56.0 | 0.0 | 41.3 | 0.3 | 12.4 |
| PBMC + LPS(5 ug/ml) + Xiidra 0.1 uM | 1st | 471.0 | 2.0 | 401.4 | 6.6 | 60.8 |
| | 2nd | 473.5 | 2.0 | 403.3 | 5.7 | 70.8 |
| | Mean | 472.3 | 2.0 | 402.4 | 6.2 | 65.8 |
| | STDER | 1.3 | 0.0 | 0.9 | 0.5 | 5.0 |
| PBMC + LPS(5 ug/ml) + Xiidra 1 uM | 1st | 619.0 | 2.0 | 510.5 | 4.8 | 106.4 |
| | 2nd | 421.0 | 2.0 | 364.4 | 4.4 | 82.8 |
| | Mean | 520.0 | 2.0 | 437.5 | 4.6 | 94.6 |
| | STDER | 99.0 | 0.0 | 73.0 | 0.2 | 11.8 |
| PBMC + LPS(5 ug/ml) + Xiidra 10 uM | 1st | 493.5 | 2.0 | 418.0 | 5.8 | 72.1 |
| | 2nd | 528.0 | 2.0 | 443.5 | 6.4 | 69.3 |
| | Mean | 510.8 | 2.0 | 430.8 | 6.1 | 70.7 |
| | STDER | 17.3 | 0.0 | 12.7 | 0.3 | 1.4 |
| PBMC + LPS(5 ug/ml) + Xiidra 100 uM | 1st | 378.0 | 2.0 | 332.4 | 7.1 | 46.8 |
| | 2nd | 397.5 | 2.0 | 346.9 | 5.8 | 59.8 |
| | Mean | 387.8 | 2.0 | 339.7 | 6.5 | 53.3 |
| | STDER | 9.8 | 0.0 | 7.3 | 0.7 | 6.5 |
| PBMC + LPS(5 ug/ml) + YY101 0.001 uM | 1st | 504.5 | 2.0 | 426.2 | 5.7 | 74.8 |
| | 2nd | 553.0 | 2.0 | 461.9 | 7.1 | 65.1 |
| | 3rd | 496.0 | 2.0 | 419.9 | 6.6 | 63.6 |
| | Mean | 517.8 | 2.0 | 436.0 | 6.5 | 67.8 |
| | STDER | 17.8 | 0.0 | 13.1 | 0.4 | 3.5 |
| PBMC + LPS(5 ug/ml) + YY101 0.01 uM | 1st | 613.0 | 2.0 | 506.1 | 6.4 | 79.1 |
| | 2nd | 572.0 | 2.0 | 475.9 | 6.0 | 79.3 |
| | 3rd | 584.0 | 2.0 | 484.7 | 6.1 | 79.5 |
| | Mean | 589.7 | 2.0 | 488.9 | 6.2 | 79.3 |
| | STDER | 12.2 | 0.0 | 9.0 | 0.1 | 0.1 |
| PBMC + LPS(5 ug/ml) + YY101 0.1 uM | 1st | 593.0 | 2.0 | 491.3 | 6.2 | 79.2 |
| | 2nd | 580.0 | 2.0 | 481.8 | 5.2 | 92.6 |
| | 3rd | 581.0 | 2.0 | 482.5 | 6.2 | 77.8 |
| | Mean | 584.7 | 2.0 | 485.2 | 5.9 | 83.2 |
| | STDER | 4.2 | 0.0 | 3.1 | 0.3 | 4.7 |
| PBMC + LPS(5 ug/ml) + YY101 1 uM | 1st | 538.5 | 2.0 | 451.2 | 6.3 | 71.6 |
| | 2nd | 506.0 | 2.0 | 427.3 | 7.1 | 60.2 |
| | 3rd | 515.5 | 2.0 | 434.3 | 6.2 | 70.0 |
| | Mean | 520.0 | 2.0 | 437.6 | 6.5 | 67.3 |
| | STDER | 9.6 | 0.0 | 7.1 | 0.3 | 3.6 |
| PBMC + LPS(5 ug/ml) + YY101 10 uM | 1st | 568.5 | 2.0 | 473.3 | 6.0 | 78.9 |
| | 2nd | 565.0 | 2.0 | 470.7 | 6.4 | 73.6 |
| | 3rd | 582.5 | 2.0 | 483.6 | 7.6 | 63.6 |
| | Mean | 572.0 | 2.0 | 475.9 | 6.7 | 72.0 |
| | STDER | 5.3 | 0.0 | 3.9 | 0.5 | 4.5 |
| PBMC + LPS(5 ug/ml) + YY101 100 uM | 1st | 816.0 | 2.0 | 656.9 | 6.3 | 104.3 |
| | 2nd | 762.0 | 2.0 | 616.5 | 6.2 | 99.4 |
| | 3rd | 759.0 | 2.0 | 614.2 | 5.0 | 122.8 |
| | Mean | 779.0 | 2.0 | 629.2 | 5.8 | 108.8 |
| | STDER | 18.5 | 0.0 | 13.9 | 0.4 | 7.1 |
| PBMC + LPS(5 ug/ml) + YDE011 0.001 uM | 1st | 383.0 | 2.0 | 336.1 | 4.5 | 74.7 |
| | 2nd | 382.0 | 2.0 | 335.4 | 5.8 | 57.8 |
| | 3rd | 416.5 | 2.0 | 361.1 | 5.8 | 62.3 |
| | Mean | 393.8 | 2.0 | 344.2 | 5.4 | 64.9 |
| | STDER | 11.3 | 0.0 | 8.4 | 0.4 | 5.0 |
| PBMC + LPS(5 ug/ml) + YDE011 0.01 uM | 1st | 392.0 | 2.0 | 342.8 | 6.3 | 54.4 |
| | 2nd | 414.0 | 2.0 | 359.2 | 6.2 | 57.9 |
| | 3rd | 444.5 | 2.0 | 381.8 | 5.1 | 74.9 |
| | Mean | 416.8 | 2.0 | 361.3 | 5.9 | 62.4 |
| | STDER | 15.2 | 0.0 | 11.3 | 0.4 | 6.3 |
| PBMC + LPS(5 ug/ml) + YDE011 0.1 uM | 1st | 378.0 | 2.0 | 332.4 | 6.6 | 50.4 |
| | 2nd | 352.0 | 2.0 | 312.9 | 5.0 | 62.6 |
| | 3rd | 398.0 | 2.0 | 347.3 | 5.7 | 60.9 |
| | Mean | 376.0 | 2.0 | 330.9 | 5.8 | 58.0 |
| | STDER | 13.3 | 0.0 | 10.0 | 0.5 | 3.8 |
| PBMC + LPS(5 ug/ml) + YDE011 1 uM | 1st | 400.5 | 2.0 | 349.2 | 5.5 | 63.5 |
| | 2nd | 363.0 | 2.0 | 321.1 | 4.8 | 66.9 |
| | 3rd | 396.0 | 2.0 | 345.8 | 4.8 | 72.0 |
| | Mean | 386.5 | 2.0 | 338.7 | 5.0 | 67.5 |
| | STDER | 11.8 | 0.0 | 8.8 | 0.2 | 2.5 |
| PBMC + LPS(5 ug/ml) + YDE011 10 uM | 1st | 410.0 | 2.0 | 356.2 | 5.4 | 66.0 |
| | 2nd | 372.0 | 2.0 | 327.9 | 5.8 | 56.5 |
| | 3rd | 396.0 | 2.0 | 345.8 | 4.7 | 73.6 |
| | Mean | 392.7 | 2.0 | 343.3 | 5.3 | 65.4 |
| | STDER | 11.1 | 0.0 | 8.3 | 0.3 | 4.9 |
| PBMC + LPS(5 ug/ml) + YDE011 100 uM | 1st | 532.0 | 2.0 | 446.4 | 6.1 | 73.2 |
| | 2nd | 472.0 | 2.0 | 402.2 | 6.1 | 65.9 |
| | 3rd | 518.0 | 2.0 | 436.1 | 6.0 | 72.7 |
| | Mean | 507.3 | 2.0 | 428.2 | 6.1 | 70.6 |
| | STDER | 18.1 | 0.0 | 13.4 | 0.0 | 2.3 |
| PBMC + LPS(5 ug/ml) + YDE043 0.001 uM | 1st | 387.0 | 2.0 | 339.1 | 4.4 | 77.1 |
| | 2nd | 2522.0 | 2.0 | 369.6 | 6.0 | 61.6 |
| | 3rd | 412.0 | 2.0 | 357.7 | 4.6 | 77.8 |
| | Mean | 1107.0 | 2.0 | 355.5 | 5.0 | 72.1 |
| | STDER | 707.5 | 0.0 | 8.9 | 0.5 | 5.3 |
| PBMC + LPS(5 ug/ml) + YDE043 0.01 uM | 1st | 405.0 | 2.0 | 352.5 | 4.5 | 78.3 |
| | 2nd | 391.5 | 2.0 | 342.5 | 6.6 | 51.9 |
| | 3rd | 429.0 | 2.0 | 370.4 | 5.0 | 74.1 |
| | Mean | 408.5 | 2.0 | 355.1 | 5.4 | 68.1 |
| | STDER | 11.0 | 0.0 | 8.2 | 0.6 | 8.2 |
| PBMC + LPS(5 ug/ml) + YDE043 0.1 uM | 1st | 417.0 | 2.0 | 361.4 | 5.0 | 72.3 |
| | 2nd | 373.5 | 2.0 | 329.0 | 5.6 | 58.8 |
| | 3rd | 445.5 | 2.0 | 382.6 | 5.6 | 68.3 |
| | Mean | 412.0 | 2.0 | 357.7 | 5.4 | 66.5 |
| | STDER | 20.9 | 0.0 | 15.6 | 0.2 | 4.0 |
| PBMC + LPS(5 ug/ml) + YDE043 1 uM | 1st | 394.5 | 2.0 | 344.7 | 5.8 | 59.4 |
| | 2nd | 378.0 | 2.0 | 332.4 | 4.8 | 69.2 |
| | 3rd | 386.5 | 2.0 | 338.7 | 5.3 | 63.9 |
| | Mean | 386.3 | 2.0 | 338.6 | 5.3 | 64.2 |
| | STDER | 4.8 | 0.0 | 3.6 | 0.3 | 2.8 |
| PBMC + LPS(5 ug/ml) + YDE043 10 uM | 1st | 411.5 | 2.0 | 357.4 | 5.2 | 68.7 |
| | 2nd | 391.0 | 2.0 | 342.1 | 5.4 | 63.3 |
| | 3rd | 403.0 | 2.0 | 351.0 | 5.6 | 62.7 |
| | Mean | 401.8 | 2.0 | 350.2 | 5.4 | 64.9 |
| | STDER | 5.9 | 0.0 | 4.4 | 0.1 | 1.9 |
| PBMC + LPS(5 ug/ml) + YDE043 100 uM | 1st | 543.0 | 2.0 | 454.5 | 6.2 | 73.3 |
| | 2nd | 488.5 | 2.0 | 414.4 | 6.4 | 64.7 |
| | Mean | 515.8 | 2.0 | 434.4 | 6.3 | 69.0 |
| | STDER | 27.3 | 0.0 | 20.1 | 0.1 | 4.3 |

TABLE 39
Bead name: IL-1 beta/IL-1F2

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.5 | 10.0 | NaN | | |
| | 2nd | −0.5 | 10.0 | NaN | | |
| | Mean | 0.0 | 10.0 | #DIV/0! | #DIV/0! | #DIV/0! |
| | STDER | 0.5 | 0.0 | #DIV/0! | #DIV/0! | #DIV/0! |
| PBMC + 0.5% DMSO | 1st | 1.5 | 10.0 | | 7.5 | 0.0 |
| | 2nd | 1.5 | 10.0 | | 7.4 | 0.0 |
| | 3rd | 1.5 | 10.0 | | 6.6 | 0.0 |
| | Mean | 1.5 | 10.0 | #DIV/0! | 7.2 | 0.0 |
| | STDER | 0.0 | 0.0 | #DIV/0! | 0.3 | 0.0 |
| PBMC + LPS(5 ug/ml) | 1st | 1906.0 | 10.0 | 7351.0 | 6.1 | 1205.1 |
| | 2nd | 1803.0 | 10.0 | 6969.3 | 5.8 | 1201.6 |
| | 3rd | 1657.5 | 10.0 | 6432.1 | 5.5 | 1169.5 |
| | Mean | 1788.8 | 10.0 | 6917.5 | 5.8 | 1192.1 |
| | STDER | 72.1 | 0.0 | 266.5 | 0.2 | 11.3 |
| PBMC + YY101 0.01 uM | 1st | 0.5 | 10.0 | | 5.2 | 0.0 |
| | 2nd | 4.5 | 10.0 | 5.5 | 4.8 | 1.2 |
| | Mean | 2.5 | 10.0 | 5.5 | 5.0 | 0.6 |
| | STDER | 2.0 | 0.0 | #DIV/0! | 0.2 | 0.6 |
| PBMC + YY101 0.1 uM | 1st | 1.5 | 10.0 | | 4.4 | 0.0 |
| | 2nd | 1.5 | 10.0 | | 5.8 | 0.0 |
| | Mean | 1.5 | 10.0 | #DIV/0! | 5.1 | 0.0 |
| | STDER | 0.0 | 0.0 | #DIV/0! | 0.7 | 0.0 |
| PBMC + YY101 1 uM | 1st | 3.5 | 10.0 | | 5.5 | 0.0 |
| | 2nd | 0.5 | 10.0 | | 5.5 | 0.0 |
| | Mean | 2.0 | 10.0 | #DIV/0! | 5.5 | 0.0 |
| | STDER | 1.5 | 0.0 | #DIV/0! | 0.0 | 0.0 |
| PBMC + YY101 10 uM | 1st | 23.5 | 10.0 | 122.9 | 6.4 | 19.2 |
| | 2nd | 2.5 | 10.0 | | 6.1 | 0.0 |
| | Mean | 13.0 | 10.0 | 122.9 | 6.3 | 9.6 |
| | STDER | 10.5 | 0.0 | #DIV/0! | 0.2 | 9.6 |
| PBMC + YY101 100 uM | 1st | 53.5 | 10.0 | 278.4 | 6.4 | 43.5 |
| | 2nd | 9.5 | 10.0 | 40.7 | 7.0 | 5.8 |
| | Mean | 31.5 | 10.0 | 159.6 | 6.7 | 24.7 |
| | STDER | 22.0 | 0.0 | 118.9 | 0.3 | 18.8 |
| PBMC + LPS(5 ug/ml) + Xiidra 0.001 uM | 1st | 2154.5 | 10.0 | 8278.0 | 6.2 | 1335.2 |
| | 2nd | 2155.5 | 10.0 | 8281.8 | 7.6 | 1089.7 |
| | Mean | 2155.0 | 10.0 | 8279.9 | 6.9 | 1212.4 |
| | STDER | 0.5 | 0.0 | 1.9 | 0.7 | 122.7 |
| PBMC + LPS(5 ug/ml) + Xiidra 0.01 uM | 1st | 2558.5 | 10.0 | 9809.5 | 5.2 | 1886.4 |
| | 2nd | 2145.5 | 10.0 | 8244.3 | 5.8 | 1421.4 |
| | Mean | 2352.0 | 10.0 | 9026.9 | 5.5 | 1653.9 |
| | STDER | 206.5 | 0.0 | 782.6 | 0.3 | 232.5 |
| PBMC + LPS(5 ug/ml) + Xiidra 0.1 uM | 1st | 2013.5 | 10.0 | 7750.8 | 6.6 | 1174.4 |
| | 2nd | 1975.5 | 10.0 | 7609.3 | 5.7 | 1335.0 |
| | Mean | 1994.5 | 10.0 | 7680.1 | 6.2 | 1254.7 |
| | STDER | 19.0 | 0.0 | 70.8 | 0.5 | 80.3 |
| PBMC + LPS(5 ug/ml) + Xiidra 1 uM | 1st | 1857.5 | 10.0 | 7171.1 | 4.8 | 1494.0 |
| | 2nd | 1850.0 | 10.0 | 7143.3 | 4.4 | 1623.5 |
| | Mean | 1853.8 | 10.0 | 7157.2 | 4.6 | 1558.7 |
| | STDER | 3.8 | 0.0 | 13.9 | 0.2 | 64.7 |
| PBMC + LPS(5 ug/ml) + Xiidra 10 uM | 1st | 1858.0 | 10.0 | 7172.9 | 5.8 | 1236.7 |
| | 2nd | 1864.5 | 10.0 | 7197.0 | 6.4 | 1124.5 |
| | Mean | 1861.3 | 10.0 | 7185.0 | 6.1 | 1180.6 |
| | STDER | 3.3 | 0.0 | 12.0 | 0.3 | 56.1 |
| PBMC + LPS(5 ug/ml) + Xiidra 100 uM | 1st | 1437.5 | 10.0 | 5623.1 | 7.1 | 792.0 |
| | 2nd | 1392.5 | 10.0 | 5457.9 | 5.8 | 941.0 |
| | Mean | 1415.0 | 10.0 | 5540.5 | 6.5 | 866.5 |
| | STDER | 22.5 | 0.0 | 82.6 | 0.7 | 74.5 |
| PBMC + LPS(5 ug/ml) + YY101 0.001 uM | 1st | 2055.5 | 10.0 | 7907.5 | 5.7 | 1387.3 |
| | 2nd | 2063.0 | 10.0 | 7935.5 | 7.1 | 1117.7 |
| | 3rd | 1811.5 | 10.0 | 7000.7 | 6.6 | 1060.7 |
| | Mean | 1976.7 | 10.0 | 7614.6 | 6.5 | 1188.6 |
| | STDER | 82.6 | 0.0 | 307.0 | 0.4 | 100.7 |
| PBMC + LPS(5 ug/ml) + YY101 0.01 uM | 1st | 2188.5 | 10.0 | 8405.7 | 6.4 | 1313.4 |
| | 2nd | 2227.5 | 10.0 | 8552.3 | 6.0 | 1425.4 |
| | 3rd | 2137.0 | 10.0 | 8212.4 | 6.1 | 1346.3 |
| | Mean | 2184.3 | 10.0 | 8390.1 | 6.2 | 1361.7 |
| | STDER | 26.2 | 0.0 | 98.4 | 0.1 | 33.2 |
| PBMC + LPS(5 ug/ml) + YY101 0.1 uM | 1st | 2151.5 | 10.0 | 8266.8 | 6.2 | 1333.4 |
| | 2nd | 2277.5 | 10.0 | 8740.8 | 5.2 | 1680.9 |
| | 3rd | 2220.5 | 10.0 | 8526.0 | 6.2 | 1375.2 |
| | Mean | 2216.5 | 10.0 | 8511.2 | 5.9 | 1463.1 |
| | STDER | 36.4 | 0.0 | 137.0 | 0.3 | 109.6 |
| PBMC + LPS(5 ug/ml) + YY101 1 uM | 1st | 2138.5 | 10.0 | 8218.0 | 6.3 | 1304.4 |
| | 2nd | 2032.5 | 10.0 | 7821.7 | 7.1 | 1101.6 |
| | 3rd | 1939.5 | 10.0 | 7475.4 | 6.2 | 1205.7 |
| | Mean | 2036.8 | 10.0 | 7838.4 | 6.5 | 1203.9 |
| | STDER | 57.5 | 0.0 | 214.5 | 0.3 | 58.6 |
| PBMC + LPS(5 ug/ml) + YY101 10 uM | 1st | 2218.5 | 10.0 | 8518.5 | 6.0 | 1419.7 |
| | 2nd | 2297.5 | 10.0 | 8816.3 | 6.4 | 1377.5 |
| | 3rd | 2251.5 | 10.0 | 8642.7 | 7.6 | 1137.2 |
| | Mean | 2255.8 | 10.0 | 8659.2 | 6.7 | 1311.5 |
| | STDER | 22.9 | 0.0 | 86.4 | 0.5 | 88.0 |
| PBMC + LPS(5 ug/ml) + YY101 100 uM | 1st | 3353.5 | 10.0 | 12944.3 | 6.3 | 2054.7 |
| | 2nd | 3064.5 | 10.0 | 11783.1 | 6.2 | 1900.5 |
| | 3rd | 3071.5 | 10.0 | 11811.0 | 5.0 | 2362.2 |
| | Mean | 3163.2 | 10.0 | 12179.5 | 5.8 | 2105.8 |
| | STDER | 95.2 | 0.0 | 382.5 | 0.4 | 135.7 |
| PBMC + LPS(5 ug/ml) + YDE011 0.001 uM | 1st | 1675.5 | 10.0 | 6498.5 | 4.5 | 1444.1 |
| | 2nd | 1688.0 | 10.0 | 6544.6 | 5.8 | 1128.4 |
| | 3rd | 1649.5 | 10.0 | 6402.7 | 5.8 | 1103.9 |
| | Mean | 1671.0 | 10.0 | 6481.9 | 5.4 | 1225.5 |
| | STDER | 11.3 | 0.0 | 41.8 | 0.4 | 109.5 |
| PBMC + LPS(5 ug/ml) + YDE011 0.01 uM | 1st | 1721.5 | 10.0 | 6668.1 | 6.3 | 1058.4 |
| | 2nd | 1691.5 | 10.0 | 6557.5 | 6.2 | 1057.7 |
| | 3rd | 1669.5 | 10.0 | 6476.4 | 5.1 | 1269.9 |
| | Mean | 1694.2 | 10.0 | 6567.3 | 5.9 | 1128.7 |
| | STDER | 15.1 | 0.0 | 55.6 | 0.4 | 70.6 |
| PBMC + LPS(5 ug/ml) + YDE011 0.1 uM | 1st | 1623.0 | 10.0 | 6305.1 | 6.6 | 955.3 |
| | 2nd | 1642.5 | 10.0 | 6376.9 | 5.0 | 1275.4 |
| | 3rd | 1636.5 | 10.0 | 6354.8 | 5.7 | 1114.9 |
| | Mean | 1634.0 | 10.0 | 6345.6 | 5.8 | 1115.2 |
| | STDER | 5.8 | 0.0 | 21.2 | 0.5 | 92.4 |
| PBMC + LPS(5 ug/ml) + YDE011 1 uM | 1st | 1690.5 | 10.0 | 6553.8 | 5.5 | 1191.6 |
| | 2nd | 1663.5 | 10.0 | 6454.2 | 4.8 | 1344.6 |
| | 3rd | 1744.5 | 10.0 | 6753.1 | 4.8 | 1406.9 |
| | Mean | 1699.5 | 10.0 | 6587.0 | 5.0 | 1314.4 |
| | STDER | 23.8 | 0.0 | 87.8 | 0.2 | 64.0 |
| PBMC + LPS(5 ug/ml) + YDE011 10 uM | 1st | 1814.0 | 10.0 | 7010.0 | 5.4 | 1298.1 |
| | 2nd | 1752.5 | 10.0 | 6782.6 | 5.8 | 1169.4 |
| | 3rd | 1813.0 | 10.0 | 7006.3 | 4.7 | 1490.7 |
| | Mean | 1793.2 | 10.0 | 6933.0 | 5.3 | 1319.4 |
| | STDER | 20.3 | 0.0 | 75.2 | 0.3 | 93.4 |
| PBMC + LPS(5 ug/ml) + YDE011 100 uM | 1st | 2213.0 | 10.0 | 8497.8 | 6.1 | 1393.1 |
| | 2nd | 2324.5 | 10.0 | 8918.4 | 6.1 | 1462.0 |
| | 3rd | 2242.5 | 10.0 | 8608.8 | 6.0 | 1434.8 |
| | Mean | 2260.0 | 10.0 | 8675.0 | 6.1 | 1430.0 |
| | STDER | 33.4 | 0.0 | 125.8 | 0.0 | 20.1 |
| PBMC + LPS(5 ug/ml) + YDE043 0.001 uM | 1st | 1640.5 | 10.0 | 6369.5 | 4.4 | 1447.6 |
| | 2nd | 1821.5 | 10.0 | 7037.7 | 6.0 | 1173.0 |
| | 3rd | 1673.5 | 10.0 | 6491.1 | 4.6 | 1411.1 |
| | Mean | 1711.8 | 10.0 | 6632.8 | 5.0 | 1343.9 |
| | STDER | 55.7 | 0.0 | 205.5 | 0.5 | 86.1 |
| PBMC + LPS(5 ug/ml) + YDE043 0.01 uM | 1st | 1657.5 | 10.0 | 6432.1 | 4.5 | 1429.4 |
| | 2nd | 1718.0 | 10.0 | 6655.2 | 6.6 | 1008.4 |
| | 3rd | 1708.5 | 10.0 | 6620.2 | 5.0 | 1324.0 |
| | Mean | 1694.7 | 10.0 | 6569.2 | 5.4 | 1253.9 |
| | STDER | 18.8 | 0.0 | 69.3 | 0.6 | 126.5 |
| PBMC + LPS(5 ug/ml) + YDE043 0.1 uM | 1st | 1553.5 | 10.0 | 6049.3 | 5.0 | 1209.9 |
| | 2nd | 1644.5 | 10.0 | 6384.2 | 5.6 | 1140.0 |
| | 3rd | 1686.5 | 10.0 | 6539.0 | 5.6 | 1167.7 |
| | Mean | 1628.2 | 10.0 | 6324.2 | 5.4 | 1172.5 |
| | STDER | 39.3 | 0.0 | 144.5 | 0.2 | 20.3 |
| PBMC + LPS(5 ug/ml) + YDE043 1 uM | 1st | 1622.5 | 10.0 | 6303.2 | 5.8 | 1086.8 |
| | 2nd | 1524.5 | 10.0 | 5942.7 | 4.8 | 1238.1 |
| | 3rd | 1632.5 | 10.0 | 6340.0 | 5.3 | 1196.2 |
| | Mean | 1593.2 | 10.0 | 6195.3 | 5.3 | 1173.7 |
| | STDER | 34.5 | 0.0 | 126.8 | 0.3 | 45.1 |
| PBMC + LPS(5 ug/ml) + YDE043 10 uM | 1st | 1644.0 | 10.0 | 6382.4 | 5.2 | 1227.4 |
| | 2nd | 1712.5 | 10.0 | 6634.9 | 5.4 | 1228.7 |
| | 3rd | 1717.5 | 10.0 | 6653.4 | 5.6 | 1188.1 |
| | Mean | 1691.3 | 10.0 | 6556.9 | 5.4 | 1214.7 |
| | STDER | 23.7 | 0.0 | 87.4 | 0.1 | 13.3 |
| PBMC + LPS(5 ug/ml) + YDE043 100 uM | 1st | 2278.5 | 10.0 | 8744.5 | 6.2 | 1410.4 |
| | 2nd | 1993.0 | 10.0 | 7674.4 | 6.4 | 1199.1 |
| | Mean | 2135.8 | 10.0 | 8209.5 | 6.3 | 1304.8 |
| | STDER | 142.8 | 0.0 | 535.0 | 0.1 | 105.6 |

TABLE 40
Bead name: TIMP-1

| | | | | Concentration | Live cell | conc/live cell |
|---|---|---|---|---|---|---|
| Sample | | MFI | Dilution | (pg/ml) | (10\hat{ }5/ml) | (pg/10\hat{ }5) |
| Blank | 1st | 0.0 | 2.0 | NaN | | |
| | 2nd | 0.0 | 2.0 | NaN | | |
| | Mean | 0.0 | 2.0 | #DIV/0! | #DIV/0! | #DIV/0! |
| | STDER | 0.0 | 0.0 | #DIV/0! | #DIV/0! | #DIV/0! |
| PBMC + 0.5% DMSO | 1st | 2918.0 | 2.0 | 4364.2 | 7.5 | 581.9 |
| | 2nd | 3072.0 | 2.0 | 4558.5 | 7.4 | 616.0 |
| | 3rd | 2936.5 | 2.0 | 4387.5 | 6.6 | 664.8 |
| | Mean | 2975.5 | 2.0 | 4436.7 | 7.2 | 620.9 |
| | STDER | 48.5 | 0.0 | 61.3 | 0.3 | 24.1 |
| PBMC + LPS(5 ug/ml) | 1st | 2018.0 | 2.0 | 3219.0 | 6.1 | 527.7 |
| | 2nd | 2060.5 | 2.0 | 3273.7 | 5.8 | 564.4 |
| | 3rd | 2159.5 | 2.0 | 3400.7 | 5.5 | 618.3 |
| | Mean | 2079.3 | 2.0 | 3297.8 | 5.8 | 570.1 |
| | STDER | 41.9 | 0.0 | 53.8 | 0.2 | 26.3 |
| PBMC + YY101 0.01 uM | 1st | 3456.0 | 2.0 | 5042.4 | 5.2 | 969.7 |
| | 2nd | 3876.0 | 2.0 | 5571.4 | 4.8 | 1160.7 |
| | Mean | 3666.0 | 2.0 | 5306.9 | 5.0 | 1065.2 |
| | STDER | 210.0 | 0.0 | 264.5 | 0.2 | 95.5 |
| PBMC + YY101 0.1 uM | 1st | 3084.0 | 2.0 | 4573.6 | 4.4 | 1039.5 |
| | 2nd | 3614.5 | 2.0 | 5242.0 | 5.8 | 903.8 |
| | Mean | 3349.3 | 2.0 | 4907.8 | 5.1 | 971.6 |
| | STDER | 265.3 | 0.0 | 334.2 | 0.7 | 67.8 |
| PBMC + YY101 1 uM | 1st | 3546.0 | 2.0 | 5155.8 | 5.5 | 937.4 |
| | 2nd | 3491.5 | 2.0 | 5087.1 | 5.5 | 924.9 |
| | Mean | 3518.8 | 2.0 | 5121.4 | 5.5 | 931.2 |
| | STDER | 27.3 | 0.0 | 34.3 | 0.0 | 6.2 |
| PBMC + YY101 10 uM | 1st | 5828.5 | 2.0 | 8049.3 | 6.4 | 1257.7 |
| | 2nd | 3505.0 | 2.0 | 5104.1 | 6.1 | 836.7 |
| | Mean | 4666.8 | 2.0 | 6576.7 | 6.3 | 1047.2 |
| | STDER | 1161.8 | 0.0 | 1472.6 | 0.2 | 210.5 |
| PBMC + YY101 100 uM | 1st | 4917.0 | 2.0 | 6886.9 | 6.4 | 1076.1 |
| | 2nd | 4391.5 | 2.0 | 6221.7 | 7.0 | 888.8 |
| | Mean | 4654.3 | 2.0 | 6554.3 | 6.7 | 982.4 |
| | STDER | 262.8 | 0.0 | 332.6 | 0.3 | 93.6 |
| PBMC + LPS(5 ug/ml) + Xiidra 0.001 uM | 1st | 1751.5 | 2.0 | 2874.2 | 6.2 | 463.6 |
| | 2nd | 1703.0 | 2.0 | 2810.9 | 7.6 | 369.9 |
| | Mean | 1727.3 | 2.0 | 2842.6 | 6.9 | 416.7 |
| | STDER | 24.3 | 0.0 | 31.6 | 0.7 | 46.9 |
| PBMC + LPS(5 ug/ml) + Xiidra 0.01 uM | 1st | 1905.5 | 2.0 | 3073.9 | 5.2 | 591.1 |
| | 2nd | 1832.5 | 2.0 | 2979.4 | 5.8 | 513.7 |
| | Mean | 1869.0 | 2.0 | 3026.7 | 5.5 | 552.4 |
| | STDER | 36.5 | 0.0 | 47.3 | 0.3 | 38.7 |
| PBMC + LPS(5 ug/ml) + Xiidra 0.1 uM | 1st | 1721.0 | 2.0 | 2834.4 | 6.6 | 429.5 |
| | 2nd | 1669.0 | 2.0 | 2766.5 | 5.7 | 485.4 |
| | Mean | 1695.0 | 2.0 | 2800.5 | 6.2 | 457.4 |
| | STDER | 26.0 | 0.0 | 34.0 | 0.5 | 27.9 |
| PBMC + LPS(5 ug/ml) + Xiidra 1 uM | 1st | 1780.5 | 2.0 | 2911.9 | 4.8 | 606.6 |
| | 2nd | 1783.0 | 2.0 | 2915.2 | 4.4 | 662.5 |
| | Mean | 1781.8 | 2.0 | 2913.5 | 4.6 | 634.6 |
| | STDER | 1.3 | 0.0 | 1.6 | 0.2 | 27.9 |
| PBMC + LPS(5 ug/ml) + Xiidra 10 uM | 1st | 1805.0 | 2.0 | 2943.7 | 5.8 | 507.5 |
| | 2nd | 1994.0 | 2.0 | 3188.1 | 6.4 | 498.1 |
| | Mean | 1899.5 | 2.0 | 3065.9 | 6.1 | 502.8 |
| | STDER | 94.5 | 0.0 | 122.2 | 0.3 | 4.7 |
| PBMC + LPS(5 ug/ml) + Xiidra 100 uM | 1st | 2052.5 | 2.0 | 3263.4 | 7.1 | 459.6 |
| | 2nd | 1981.5 | 2.0 | 3172.0 | 5.8 | 546.9 |
| | Mean | 2017.0 | 2.0 | 3217.7 | 6.5 | 503.3 |
| | STDER | 35.5 | 0.0 | 45.7 | 0.7 | 43.6 |
| PBMC + LPS(5 ug/ml) + YY101 0.001 uM | 1st | 1596.0 | 2.0 | 2670.8 | 5.7 | 468.6 |
| | 2nd | 1464.5 | 2.0 | 2497.3 | 7.1 | 351.7 |
| | 3rd | 1648.5 | 2.0 | 2739.7 | 6.6 | 415.1 |
| | Mean | 1569.7 | 2.0 | 2635.9 | 6.5 | 411.8 |
| | STDER | 54.7 | 0.0 | 72.1 | 0.4 | 33.8 |
| PBMC + LPS(5 ug/ml) + YY101 0.01 uM | 1st | 1569.5 | 2.0 | 2636.0 | 6.4 | 411.9 |
| | 2nd | 1506.0 | 2.0 | 2552.3 | 6.0 | 425.4 |
| | 3rd | 1668.0 | 2.0 | 2765.2 | 6.1 | 453.3 |
| | Mean | 1581.2 | 2.0 | 2651.1 | 6.2 | 430.2 |
| | STDER | 47.1 | 0.0 | 61.9 | 0.1 | 12.2 |
| PBMC + LPS(5 ug/ml) + YY101 0.1 uM | 1st | 1634.5 | 2.0 | 2721.3 | 6.2 | 438.9 |
| | 2nd | 1465.5 | 2.0 | 2498.6 | 5.2 | 480.5 |
| | 3rd | 1526.0 | 2.0 | 2578.7 | 6.2 | 415.9 |
| | Mean | 1542.0 | 2.0 | 2599.6 | 5.9 | 445.1 |
| | STDER | 49.4 | 0.0 | 65.1 | 0.3 | 18.9 |
| PBMC + LPS(5 ug/ml) + YY101 1 uM | 1st | 1553.5 | 2.0 | 2614.9 | 6.3 | 415.1 |
| | 2nd | 1585.0 | 2.0 | 2656.4 | 7.1 | 374.1 |
| | 3rd | 1654.5 | 2.0 | 2747.5 | 6.2 | 443.2 |
| | Mean | 1597.7 | 2.0 | 2672.9 | 6.5 | 410.8 |
| | STDER | 29.8 | 0.0 | 39.2 | 0.3 | 20.0 |
| PBMC + LPS(5 ug/ml) + YY101 10 uM | 1st | 1650.0 | 2.0 | 2741.7 | 6.0 | 456.9 |
| | 2nd | 1695.5 | 2.0 | 2801.1 | 6.4 | 437.7 |
| | 3rd | 1495.5 | 2.0 | 2538.4 | 7.6 | 334.0 |
| | Mean | 1613.7 | 2.0 | 2693.7 | 6.7 | 409.5 |
| | STDER | 60.5 | 0.0 | 79.6 | 0.5 | 38.2 |
| PBMC + LPS(5 ug/ml) + YY101 100 uM | 1st | 2179.0 | 2.0 | 3425.7 | 6.3 | 543.8 |
| | 2nd | 2135.0 | 2.0 | 3369.3 | 6.2 | 543.4 |
| | 3rd | 1987.0 | 2.0 | 3179.1 | 5.0 | 635.8 |
| | Mean | 2100.3 | 2.0 | 3324.7 | 5.8 | 574.3 |
| | STDER | 58.1 | 0.0 | 74.6 | 0.4 | 30.7 |
| PBMC + LPS(5 ug/ml) + YDE011 0.001 uM | 1st | 974.0 | 2.0 | 1831.6 | 4.5 | 407.0 |
| | 2nd | 852.5 | 2.0 | 1659.9 | 5.8 | 286.2 |
| | 3rd | 1013.5 | 2.0 | 1886.6 | 5.8 | 325.3 |
| | Mean | 946.7 | 2.0 | 1792.7 | 5.4 | 339.5 |
| | STDER | 48.4 | 0.0 | 68.3 | 0.4 | 35.6 |
| PBMC + LPS(5 ug/ml) + YDE011 0.01 uM | 1st | 936.0 | 2.0 | 1778.3 | 6.3 | 282.3 |
| | 2nd | 959.5 | 2.0 | 1811.3 | 6.2 | 292.1 |
| | 3rd | 1132.0 | 2.0 | 2050.0 | 5.1 | 402.0 |
| | Mean | 1009.2 | 2.0 | 1879.9 | 5.9 | 325.5 |
| | STDER | 61.8 | 0.0 | 85.6 | 0.4 | 38.4 |
| PBMC + LPS(5 ug/ml) + YDE011 0.1 uM | 1st | 1008.5 | 2.0 | 1879.7 | 6.6 | 284.8 |
| | 2nd | 865.0 | 2.0 | 1677.8 | 5.0 | 335.6 |
| | 3rd | 1049.0 | 2.0 | 1935.9 | 5.7 | 339.6 |
| | Mean | 974.2 | 2.0 | 1831.1 | 5.8 | 320.0 |
| | STDER | 55.8 | 0.0 | 78.4 | 0.5 | 17.6 |
| PBMC + LPS(5 ug/ml) + YDE011 1 uM | 1st | 1001.5 | 2.0 | 1869.9 | 5.5 | 340.0 |
| | 2nd | 916.5 | 2.0 | 1750.8 | 4.8 | 364.7 |
| | 3rd | 918.5 | 2.0 | 1753.6 | 4.8 | 365.3 |
| | Mean | 945.5 | 2.0 | 1791.4 | 5.0 | 356.7 |
| | STDER | 28.0 | 0.0 | 39.3 | 0.2 | 8.4 |
| PBMC + LPS(5 ug/ml) + YDE011 10 uM | 1st | 966.0 | 2.0 | 1820.4 | 5.4 | 337.1 |
| | 2nd | 947.5 | 2.0 | 1794.4 | 5.8 | 309.4 |
| | 3rd | 873.5 | 2.0 | 1689.9 | 4.7 | 359.5 |
| | Mean | 929.0 | 2.0 | 1768.2 | 5.3 | 335.3 |
| | STDER | 28.3 | 0.0 | 39.9 | 0.3 | 14.5 |
| PBMC + LPS(5 ug/ml) + YDE011 100 uM | 1st | 1336.5 | 2.0 | 2326.8 | 6.1 | 381.4 |
| | 2nd | 1184.5 | 2.0 | 2121.7 | 6.1 | 347.8 |
| | 3rd | 1172.0 | 2.0 | 2104.6 | 6.0 | 350.8 |
| | Mean | 1231.0 | 2.0 | 2184.4 | 6.1 | 360.0 |
| | STDER | 52.9 | 0.0 | 71.4 | 0.0 | 10.7 |
| PBMC + LPS(5 ug/ml) + YDE043 0.001 uM | 1st | 1014.0 | 2.0 | 1887.3 | 4.4 | 428.9 |
| | 2nd | 1129.5 | 2.0 | 2046.6 | 6.0 | 341.1 |
| | 3rd | 1147.0 | 2.0 | 2070.5 | 4.6 | 450.1 |
| | Mean | 1096.8 | 2.0 | 2001.5 | 5.0 | 406.7 |
| | STDER | 41.7 | 0.0 | 57.5 | 0.5 | 33.4 |
| PBMC + LPS(5 ug/ml) + YDE043 0.01 uM | 1st | 1101.0 | 2.0 | 2007.5 | 4.5 | 446.1 |
| | 2nd | 1089.5 | 2.0 | 1991.7 | 6.6 | 301.8 |
| | 3rd | 1067.0 | 2.0 | 1960.7 | 5.0 | 392.1 |
| | Mean | 1085.8 | 2.0 | 1986.7 | 5.4 | 380.0 |
| | STDER | 10.0 | 0.0 | 13.7 | 0.6 | 42.1 |
| PBMC + LPS(5 ug/ml) + YDE043 0.1 uM | 1st | 987.5 | 2.0 | 1850.4 | 5.0 | 370.1 |
| | 2nd | 893.5 | 2.0 | 1718.3 | 5.6 | 306.8 |
| | 3rd | 1037.5 | 2.0 | 1919.9 | 5.6 | 342.8 |
| | Mean | 972.8 | 2.0 | 1829.5 | 5.4 | 339.9 |
| | STDER | 42.2 | 0.0 | 59.2 | 0.2 | 18.3 |
| PBMC + LPS(5 ug/ml) + YDE043 1 uM | 1st | 1043.5 | 2.0 | 1928.2 | 5.8 | 332.5 |
| | 2nd | 958.5 | 2.0 | 1809.9 | 4.8 | 377.1 |
| | 3rd | 935.0 | 2.0 | 1776.9 | 5.3 | 335.3 |
| | Mean | 979.0 | 2.0 | 1838.3 | 5.3 | 348.3 |
| | STDER | 33.0 | 0.0 | 46.0 | 0.3 | 14.4 |
| PBMC + LPS(5 ug/ml) + YDE043 10 uM | 1st | 990.0 | 2.0 | 1853.9 | 5.2 | 356.5 |
| | 2nd | 1032.0 | 2.0 | 1912.3 | 5.4 | 354.1 |
| | 3rd | 929.0 | 2.0 | 1768.4 | 5.6 | 315.8 |
| | Mean | 983.7 | 2.0 | 1844.9 | 5.4 | 342.1 |
| | STDER | 29.9 | 0.0 | 41.8 | 0.1 | 13.2 |
| PBMC + LPS(5 ug/ml) + YDE043 100 uM | 1st | 1277.5 | 2.0 | 2247.5 | 6.2 | 362.5 |
| | 2nd | 1143.0 | 2.0 | 2065.1 | 6.4 | 322.7 |
| | Mean | 1210.3 | 2.0 | 2156.3 | 6.3 | 342.6 |
| | STDER | 67.3 | 0.0 | 91.2 | 0.1 | 19.9 |

TABLE 41
Bead name: GM-CSF

| | | | | | | conc/ |
|---|---|---|---|---|---|---|
| | | | | Concentration | Live cell | live cell |
| Sample | | MFI | Dilution | (pg/ml) | (10\hat{ }5/ml) | (pg/10\hat{ }5) |
| Blank | 1st | −1.5 | 2.0 | NaN | | |
| | 2nd | 1.5 | 2.0 | NaN | | |
| | Mean | 0.0 | 2.0 | #DIV/0! | #DIV/0! | #DIV/0! |
| | STDER | 1.5 | 0.0 | #DIV/0! | #DIV/0! | #DIV/0! |
| PBMC + 0.5% DMSO | 1st | 1.5 | 2.0 | | 7.5 | 0.0 |
| | 2nd | 3.5 | 2.0 | | 7.4 | 0.0 |
| | 3rd | 2.5 | 2.0 | | 6.6 | 0.0 |
| | Mean | 2.5 | 2.0 | #DIV/0! | 7.2 | 0.0 |
| | STDER | 0.6 | 0.0 | #DIV/0! | 0.3 | 0.0 |
| PBMC + LPS(5 ug/ml) | 1st | 142.5 | 2.0 | 123.7 | 6.1 | 20.3 |
| | 2nd | 133.5 | 2.0 | 116.3 | 5.8 | 20.0 |
| | 3rd | 132.5 | 2.0 | 115.4 | 5.5 | 21.0 |
| | Mean | 136.2 | 2.0 | 118.5 | 5.8 | 20.4 |
| | STDER | 3.2 | 0.0 | 2.6 | 0.2 | 0.3 |
| PBMC + YY101 0.01 uM | 1st | 8.5 | 2.0 | 5.3 | 5.2 | 1.0 |
| | 2nd | 9.5 | 2.0 | 6.3 | 4.8 | 1.3 |
| | Mean | 9.0 | 2.0 | 5.8 | 5.0 | 1.2 |
| | STDER | 0.5 | 0.0 | 0.5 | 0.2 | 0.2 |
| PBMC + YY101 0.1 uM | 1st | 5.5 | 2.0 | 1.9 | 4.4 | 0.4 |
| | 2nd | 10.0 | 2.0 | 6.9 | 5.8 | 1.2 |
| | Mean | 7.8 | 2.0 | 4.4 | 5.1 | 0.8 |
| | STDER | 2.3 | 0.0 | 2.5 | 0.7 | 0.4 |
| PBMC + YY101 1 uM | 1st | 8.0 | 2.0 | 4.7 | 5.5 | 0.9 |
| | 2nd | 7.5 | 2.0 | 4.2 | 5.5 | 0.8 |
| | Mean | 7.8 | 2.0 | 4.4 | 5.5 | 0.8 |
| | STDER | 0.3 | 0.0 | 0.3 | 0.0 | 0.1 |
| PBMC + YY101 10 uM | 1st | 37.5 | 2.0 | 33.5 | 6.4 | 5.2 |
| | 2nd | 16.5 | 2.0 | 13.5 | 6.1 | 2.2 |
| | Mean | 27.0 | 2.0 | 23.5 | 6.3 | 3.7 |
| | STDER | 10.5 | 0.0 | 10.0 | 0.2 | 1.5 |
| PBMC + YY101 100 uM | 1st | 17.5 | 2.0 | 14.5 | 6.4 | 2.3 |
| | 2nd | 10.5 | 2.0 | 7.4 | 7.0 | 1.1 |
| | Mean | 14.0 | 2.0 | 11.0 | 6.7 | 1.7 |
| | STDER | 3.5 | 0.0 | 3.6 | 0.3 | 0.6 |
| PBMC + LPS(5 ug/ml) + Xiidra 0.001 uM | 1st | 174.5 | 2.0 | 149.9 | 6.2 | 24.2 |
| | 2nd | 160.5 | 2.0 | 138.5 | 7.6 | 18.2 |
| | Mean | 167.5 | 2.0 | 144.2 | 6.9 | 21.2 |
| | STDER | 7.0 | 0.0 | 5.7 | 0.7 | 3.0 |
| PBMC + LPS(5 ug/ml) + Xiidra 0.01 uM | 1st | 193.5 | 2.0 | 165.2 | 5.2 | 31.8 |
| | 2nd | 189.5 | 2.0 | 162.0 | 5.8 | 27.9 |
| | Mean | 191.5 | 2.0 | 163.6 | 5.5 | 29.9 |
| | STDER | 2.0 | 0.0 | 1.6 | 0.3 | 1.9 |
| PBMC + LPS(5 ug/ml) + Xiidra 0.1 uM | 1st | 124.5 | 2.0 | 108.8 | 6.6 | 16.5 |
| | 2nd | 133.0 | 2.0 | 115.9 | 5.7 | 20.3 |
| | Mean | 128.8 | 2.0 | 112.3 | 6.2 | 18.4 |
| | STDER | 4.3 | 0.0 | 3.5 | 0.5 | 1.9 |
| PBMC + LPS(5 ug/ml) + Xiidra 1 uM | 1st | 125.5 | 2.0 | 109.6 | 4.8 | 22.8 |
| | 2nd | 109.0 | 2.0 | 95.8 | 4.4 | 21.8 |
| | Mean | 117.3 | 2.0 | 102.7 | 4.6 | 22.3 |
| | STDER | 8.3 | 0.0 | 6.9 | 0.2 | 0.5 |
| PBMC + LPS(5 ug/ml) + Xiidra 10 uM | 1st | 97.5 | 2.0 | 86.1 | 5.8 | 14.9 |
| | 2nd | 103.5 | 2.0 | 91.2 | 6.4 | 14.3 |
| | Mean | 100.5 | 2.0 | 88.7 | 6.1 | 14.6 |
| | STDER | 3.0 | 0.0 | 2.5 | 0.3 | 0.3 |
| PBMC + LPS(5 ug/ml) + Xiidra 100 uM | 1st | 96.5 | 2.0 | 85.3 | 7.1 | 12.0 |
| | 2nd | 98.5 | 2.0 | 87.0 | 5.8 | 15.0 |
| | Mean | 97.5 | 2.0 | 86.1 | 6.5 | 13.5 |
| | STDER | 1.0 | 0.0 | 0.8 | 0.7 | 1.5 |
| PBMC + LPS(5 ug/ml) + YY101 0.001 uM | 1st | 161.0 | 2.0 | 138.9 | 5.7 | 24.4 |
| | 2nd | 187.5 | 2.0 | 160.4 | 7.1 | 22.6 |
| | 3rd | 212.0 | 2.0 | 180.1 | 6.6 | 27.3 |
| | Mean | 186.8 | 2.0 | 159.8 | 6.5 | 24.7 |
| | STDER | 14.7 | 0.0 | 11.9 | 0.4 | 1.4 |
| PBMC + LPS(5 ug/ml) + YY101 0.01 uM | 1st | 192.5 | 2.0 | 164.4 | 6.4 | 25.7 |
| | 2nd | 186.5 | 2.0 | 159.6 | 6.0 | 26.6 |
| | 3rd | 178.5 | 2.0 | 153.1 | 6.1 | 25.1 |
| | Mean | 185.8 | 2.0 | 159.0 | 6.2 | 25.8 |
| | STDER | 4.1 | 0.0 | 3.3 | 0.1 | 0.4 |
| PBMC + LPS(5 ug/ml) + YY101 0.1 uM | 1st | 175.5 | 2.0 | 150.7 | 6.2 | 24.3 |
| | 2nd | 157.0 | 2.0 | 135.6 | 5.2 | 26.1 |
| | 3rd | 153.5 | 2.0 | 132.7 | 6.2 | 21.4 |
| | Mean | 162.0 | 2.0 | 139.7 | 5.9 | 23.9 |
| | STDER | 6.8 | 0.0 | 5.6 | 0.3 | 1.4 |
| PBMC + LPS(5 ug/ml) + YY101 1 uM | 1st | 159.0 | 2.0 | 137.2 | 6.3 | 21.8 |
| | 2nd | 154.0 | 2.0 | 133.1 | 7.1 | 18.8 |
| | 3rd | 178.5 | 2.0 | 153.1 | 6.2 | 24.7 |
| | Mean | 163.8 | 2.0 | 141.2 | 6.5 | 21.7 |
| | STDER | 7.5 | 0.0 | 6.1 | 0.3 | 1.7 |
| PBMC + LPS(5 ug/ml) + YY101 10 uM | 1st | 178.5 | 2.0 | 153.1 | 6.0 | 25.5 |
| | 2nd | 167.0 | 2.0 | 143.8 | 6.4 | 22.5 |
| | 3rd | 171.5 | 2.0 | 147.4 | 7.6 | 19.4 |
| | Mean | 172.3 | 2.0 | 148.1 | 6.7 | 22.5 |
| | STDER | 3.3 | 0.0 | 2.7 | 0.5 | 1.8 |
| PBMC + LPS(5 ug/ml) + YY101 100 uM | 1st | 287.0 | 2.0 | 239.6 | 6.3 | 38.0 |
| | 2nd | 257.5 | 2.0 | 216.3 | 6.2 | 34.9 |
| | 3rd | 257.5 | 2.0 | 216.3 | 5.0 | 43.3 |
| | Mean | 267.3 | 2.0 | 224.0 | 5.8 | 38.7 |
| | STDER | 9.8 | 0.0 | 7.8 | 0.4 | 2.4 |
| PBMC + LPS(5 ug/ml) + YDE011 0.001 uM | 1st | 105.0 | 2.0 | 92.5 | 4.5 | 20.6 |
| | 2nd | 109.5 | 2.0 | 96.3 | 5.8 | 16.6 |
| | 3rd | 116.5 | 2.0 | 102.1 | 5.8 | 17.6 |
| | Mean | 110.3 | 2.0 | 97.0 | 5.4 | 18.3 |
| | STDER | 3.3 | 0.0 | 2.8 | 0.4 | 1.2 |
| PBMC + LPS(5 ug/ml) + YDE011 0.01 uM | 1st | 102.5 | 2.0 | 90.4 | 6.3 | 14.3 |
| | 2nd | 123.5 | 2.0 | 108.0 | 6.2 | 17.4 |
| | 3rd | 127.5 | 2.0 | 111.3 | 5.1 | 21.8 |
| | Mean | 117.8 | 2.0 | 103.2 | 5.9 | 17.9 |
| | STDER | 7.8 | 0.0 | 6.5 | 0.4 | 2.2 |
| PBMC + LPS(5 ug/ml) + YDE011 0.1 uM | 1st | 114.5 | 2.0 | 100.5 | 6.6 | 15.2 |
| | 2nd | 103.0 | 2.0 | 90.8 | 5.0 | 18.2 |
| | 3rd | 108.5 | 2.0 | 95.4 | 5.7 | 16.7 |
| | Mean | 108.7 | 2.0 | 95.6 | 5.8 | 16.7 |
| | STDER | 3.3 | 0.0 | 2.8 | 0.5 | 0.8 |
| PBMC + LPS(5 ug/ml) + YDE011 1 uM | 1st | 109.5 | 2.0 | 96.3 | 5.5 | 17.5 |
| | 2nd | 105.0 | 2.0 | 92.5 | 4.8 | 19.3 |
| | 3rd | 105.5 | 2.0 | 92.9 | 4.8 | 19.4 |
| | Mean | 106.7 | 2.0 | 93.9 | 5.0 | 18.7 |
| | STDER | 1.4 | 0.0 | 1.2 | 0.2 | 0.6 |
| PBMC + LPS(5 ug/ml) + YDE011 10 uM | 1st | 117.5 | 2.0 | 103.0 | 5.4 | 19.1 |
| | 2nd | 105.5 | 2.0 | 92.9 | 5.8 | 16.0 |
| | 3rd | 112.5 | 2.0 | 98.8 | 4.7 | 21.0 |
| | Mean | 111.8 | 2.0 | 98.2 | 5.3 | 18.7 |
| | STDER | 3.5 | 0.0 | 2.9 | 0.3 | 1.5 |
| PBMC + LPS(5 ug/ml) + YDE011 100 uM | 1st | 173.5 | 2.0 | 149.0 | 6.1 | 24.4 |
| | 2nd | 147.5 | 2.0 | 127.8 | 6.1 | 21.0 |
| | 3rd | 150.5 | 2.0 | 130.3 | 6.0 | 21.7 |
| | Mean | 157.2 | 2.0 | 135.7 | 6.1 | 22.4 |
| | STDER | 8.2 | 0.0 | 6.7 | 0.0 | 1.1 |
| PBMC + LPS(5 ug/ml) + YDE043 0.001 uM | 1st | 96.5 | 2.0 | 85.3 | 4.4 | 19.4 |
| | 2nd | 118.5 | 2.0 | 103.8 | 6.0 | 17.3 |
| | 3rd | 115.5 | 2.0 | 101.3 | 4.6 | 22.0 |
| | Mean | 110.2 | 2.0 | 96.8 | 5.0 | 19.6 |
| | STDER | 6.9 | 0.0 | 5.8 | 0.5 | 1.4 |
| PBMC + LPS(5 ug/ml) + YDE043 0.01 uM | 1st | 107.5 | 2.0 | 94.6 | 4.5 | 21.0 |
| | 2nd | 97.5 | 2.0 | 86.1 | 6.6 | 13.1 |
| | 3rd | 111.5 | 2.0 | 97.9 | 5.0 | 19.6 |
| | Mean | 105.5 | 2.0 | 92.9 | 5.4 | 17.9 |
| | STDER | 4.2 | 0.0 | 3.5 | 0.6 | 2.5 |
| PBMC + LPS(5 ug/ml) + YDE043 0.1 uM | 1st | 99.5 | 2.0 | 87.8 | 5.0 | 17.6 |
| | 2nd | 103.5 | 2.0 | 91.2 | 5.6 | 16.3 |
| | 3rd | 112.0 | 2.0 | 98.4 | 5.6 | 17.6 |
| | Mean | 105.0 | 2.0 | 92.5 | 5.4 | 17.1 |
| | STDER | 3.7 | 0.0 | 3.1 | 0.2 | 0.4 |
| PBMC + LPS(5 ug/ml) + YDE043 1 uM | 1st | 101.5 | 2.0 | 89.5 | 5.8 | 15.4 |
| | 2nd | 104.5 | 2.0 | 92.1 | 4.8 | 19.2 |
| | 3rd | 106.0 | 2.0 | 93.3 | 5.3 | 17.6 |
| | Mean | 104.0 | 2.0 | 91.6 | 5.3 | 17.4 |
| | STDER | 1.3 | 0.0 | 1.1 | 0.3 | 1.1 |
| PBMC + LPS(5 ug/ml) + YDE043 10 uM | 1st | 116.5 | 2.0 | 102.1 | 5.2 | 19.6 |
| | 2nd | 109.5 | 2.0 | 96.3 | 5.4 | 17.8 |
| | 3rd | 119.5 | 2.0 | 104.6 | 5.6 | 18.7 |
| | Mean | 115.2 | 2.0 | 101.0 | 5.4 | 18.7 |
| | STDER | 3.0 | 0.0 | 2.5 | 0.1 | 0.5 |
| PBMC + LPS(5 ug/ml) + YDE043 100 uM | 1st | 162.5 | 2.0 | 140.1 | 6.2 | 22.6 |
| | 2nd | 155.5 | 2.0 | 134.4 | 6.4 | 21.0 |
| | Mean | 159.0 | 2.0 | 137.2 | 6.3 | 21.8 |
| | STDER | 3.5 | 0.0 | 2.9 | 0.1 | 0.8 |

TABLE 42
Bead name: IL-2

| | | | | | | conc/ |
|---|---|---|---|---|---|---|
| | | | | Concentration | Live cell | live cell |
| Sample | | MFI | Dilution | (pg/ml) | (10\hat{ }5/ml) | (pg/10\hat{ }5) |
| Blank | 1st | 0.0 | 2.0 | NaN | | |
| | 2nd | 0.0 | 2.0 | NaN | | |
| | Mean | 0.0 | 2.0 | #DIV/0! | #DIV/0! | #DIV/0! |
| | STDER | 0.0 | 0.0 | #DIV/0! | #DIV/0! | #DIV/0! |
| PBMC + 0.5% DMSO | 1st | 11.5 | 2.0 | 2.3 | 7.5 | 0.3 |
| | 2nd | 14.5 | 2.0 | 4.4 | 7.4 | 0.6 |
| | 3rd | 12.0 | 2.0 | 2.6 | 6.6 | 0.4 |
| | Mean | 12.7 | 2.0 | 3.1 | 7.2 | 0.4 |
| | STDER | 0.9 | 0.0 | 0.7 | 0.3 | 0.1 |
| PBMC + LPS(5 ug/ml) | 1st | 26.0 | 2.0 | 12.5 | 6.1 | 2.1 |
| | 2nd | 23.5 | 2.0 | 10.8 | 5.8 | 1.9 |
| | 3rd | 26.0 | 2.0 | 12.5 | 5.5 | 2.3 |
| | Mean | 25.2 | 2.0 | 11.9 | 5.8 | 2.1 |
| | STDER | 0.8 | 0.0 | 0.6 | 0.2 | 0.1 |
| PBMC + YY101 0.01 uM | 1st | 92.5 | 2.0 | 56.9 | 5.2 | 10.9 |
| | 2nd | 94.0 | 2.0 | 57.9 | 4.8 | 12.1 |
| | Mean | 93.3 | 2.0 | 57.4 | 5.0 | 11.5 |
| | STDER | 0.8 | 0.0 | 0.5 | 0.2 | 0.6 |
| PBMC + YY101 0.1 uM | 1st | 75.0 | 2.0 | 45.4 | 4.4 | 10.3 |
| | 2nd | 117.5 | 2.0 | 73.2 | 5.8 | 12.6 |
| | Mean | 96.3 | 2.0 | 59.3 | 5.1 | 11.5 |
| | STDER | 21.3 | 0.0 | 13.9 | 0.7 | 1.1 |
| PBMC + YY101 1 uM | 1st | 97.0 | 2.0 | 59.8 | 5.5 | 10.9 |
| | 2nd | 106.0 | 2.0 | 65.7 | 5.5 | 11.9 |
| | Mean | 101.5 | 2.0 | 62.8 | 5.5 | 11.4 |
| | STDER | 4.5 | 0.0 | 2.9 | 0.0 | 0.5 |
| PBMC + YY101 10 uM | 1st | 112.0 | 2.0 | 69.6 | 6.4 | 10.9 |
| | 2nd | 115.0 | 2.0 | 71.5 | 6.1 | 11.7 |
| | Mean | 113.5 | 2.0 | 70.6 | 6.3 | 11.3 |
| | STDER | 1.5 | 0.0 | 1.0 | 0.2 | 0.4 |
| PBMC + YY101 100 uM | 1st | 27.0 | 2.0 | 13.2 | 6.4 | 2.1 |
| | 2nd | 31.0 | 2.0 | 16.0 | 7.0 | 2.3 |
| | Mean | 29.0 | 2.0 | 14.6 | 6.7 | 2.2 |
| | STDER | 2.0 | 0.0 | 1.4 | 0.3 | 0.1 |
| PBMC + LPS(5 ug/ml) + Xiidra 0.001 uM | 1st | 25.0 | 2.0 | 11.8 | 6.2 | 1.9 |
| | 2nd | 16.0 | 2.0 | 5.5 | 7.6 | 0.7 |
| | Mean | 20.5 | 2.0 | 8.7 | 6.9 | 1.3 |
| | STDER | 4.5 | 0.0 | 3.2 | 0.7 | 0.6 |
| PBMC + LPS(5 ug/ml) + Xiidra 0.01 uM | 1st | 14.0 | 2.0 | 4.1 | 5.2 | 0.8 |
| | 2nd | 9.0 | 2.0 | 0.4 | 5.8 | 0.1 |
| | Mean | 11.5 | 2.0 | 2.2 | 5.5 | 0.4 |
| | STDER | 2.5 | 0.0 | 1.9 | 0.3 | 0.4 |
| PBMC + LPS(5 ug/ml) + Xiidra 0.1 uM | 1st | 6.0 | 2.0 | | 6.6 | 0.0 |
| | 2nd | 10.0 | 2.0 | 1.1 | 5.7 | 0.2 |
| | Mean | 8.0 | 2.0 | 1.1 | 6.2 | 0.1 |
| | STDER | 2.0 | 0.0 | #DIV/0! | 0.5 | 0.1 |
| PBMC + LPS(5 ug/ml) + Xiidra 1 uM | 1st | 6.0 | 2.0 | | 4.8 | 0.0 |
| | 2nd | 3.0 | 2.0 | | 4.4 | 0.0 |
| | Mean | 4.5 | 2.0 | #DIV/0! | 4.6 | 0.0 |
| | STDER | 1.5 | 0.0 | #DIV/0! | 0.2 | 0.0 |
| PBMC + LPS(5 ug/ml) + Xiidra 10 uM | 1st | 5.0 | 2.0 | | 5.8 | 0.0 |
| | 2nd | 4.0 | 2.0 | | 6.4 | 0.0 |
| | Mean | 4.5 | 2.0 | #DIV/0! | 6.1 | 0.0 |
| | STDER | 0.5 | 0.0 | #DIV/0! | 0.3 | 0.0 |
| PBMC + LPS(5 ug/ml) + Xiidra 100 uM | 1st | 2.0 | 2.0 | | 7.1 | 0.0 |
| | 2nd | 3.0 | 2.0 | | 5.8 | 0.0 |
| | Mean | 2.5 | 2.0 | #DIV/0! | 6.5 | 0.0 |
| | STDER | 0.5 | 0.0 | #DIV/0! | 0.7 | 0.0 |
| PBMC + LPS(5 ug/ml) + YY101 0.001 uM | 1st | 13.0 | 2.0 | 3.4 | 5.7 | 0.6 |
| | 2nd | 19.0 | 2.0 | 7.6 | 7.1 | 1.1 |
| | 3rd | 17.0 | 2.0 | 6.2 | 6.6 | 0.9 |
| | Mean | 16.3 | 2.0 | 5.7 | 6.5 | 0.9 |
| | STDER | 1.8 | 0.0 | 1.3 | 0.4 | 0.1 |
| PBMC + LPS(5 ug/ml) + YY101 0.01 uM | 1st | 18.0 | 2.0 | 6.9 | 6.4 | 1.1 |
| | 2nd | 25.0 | 2.0 | 11.8 | 6.0 | 2.0 |
| | 3rd | 18.0 | 2.0 | 6.9 | 6.1 | 1.1 |
| | Mean | 20.3 | 2.0 | 8.6 | 6.2 | 1.4 |
| | STDER | 2.3 | 0.0 | 1.6 | 0.1 | 0.3 |
| PBMC + LPS(5 ug/ml) + YY101 0.1 uM | 1st | 8.0 | 2.0 | | 6.2 | 0.0 |
| | 2nd | 3.0 | 2.0 | | 5.2 | 0.0 |
| | 3rd | 8.0 | 2.0 | | 6.2 | 0.0 |
| | Mean | 6.3 | 2.0 | #DIV/0! | 5.9 | 0.0 |
| | STDER | 1.7 | 0.0 | #DIV/0! | 0.3 | 0.0 |
| PBMC + LPS(5 ug/ml) + YY101 1 uM | 1st | 15.5 | 2.0 | 5.2 | 6.3 | 0.8 |
| | 2nd | 13.5 | 2.0 | 3.7 | 7.1 | 0.5 |
| | 3rd | 12.0 | 2.0 | 2.6 | 6.2 | 0.4 |
| | Mean | 13.7 | 2.0 | 3.8 | 6.5 | 0.6 |
| | STDER | 1.0 | 0.0 | 0.7 | 0.3 | 0.1 |
| PBMC + LPS(5 ug/ml) + YY101 10 uM | 1st | 19.5 | 2.0 | 8.0 | 6.0 | 1.3 |
| | 2nd | 14.0 | 2.0 | 4.1 | 6.4 | 0.6 |
| | 3rd | 13.5 | 2.0 | 3.7 | 7.6 | 0.5 |
| | Mean | 15.7 | 2.0 | 5.3 | 6.7 | 0.8 |
| | STDER | 1.9 | 0.0 | 1.4 | 0.5 | 0.3 |
| PBMC + LPS(5 ug/ml) + YY101 100 uM | 1st | 11.0 | 2.0 | 1.9 | 6.3 | 0.3 |
| | 2nd | 9.0 | 2.0 | 0.4 | 6.2 | 0.1 |
| | 3rd | 7.0 | 2.0 | | 5.0 | 0.0 |
| | Mean | 9.0 | 2.0 | 1.1 | 5.8 | 0.1 |
| | STDER | 1.2 | 0.0 | 0.8 | 0.4 | 0.1 |
| PBMC + LPS(5 ug/ml) + YDE011 0.001 uM | 1st | 4.0 | 2.0 | | 4.5 | 0.0 |
| | 2nd | 4.0 | 2.0 | | 5.8 | 0.0 |
| | 3rd | 4.0 | 2.0 | | 5.8 | 0.0 |
| | Mean | 4.0 | 2.0 | #DIV/0! | 5.4 | 0.0 |
| | STDER | 0.0 | 0.0 | #DIV/0! | 0.4 | 0.0 |
| PBMC + LPS(5 ug/ml) + YDE011 0.01 uM | 1st | 5.0 | 2.0 | | 6.3 | 0.0 |
| | 2nd | 5.0 | 2.0 | | 6.2 | 0.0 |
| | 3rd | 5.0 | 2.0 | | 5.1 | 0.0 |
| | Mean | 5.0 | 2.0 | #DIV/0! | 5.9 | 0.0 |
| | STDER | 0.0 | 0.0 | #DIV/0! | 0.4 | 0.0 |
| PBMC + LPS(5 ug/ml) + YDE011 0.1 uM | 1st | 7.0 | 2.0 | | 6.6 | 0.0 |
| | 2nd | 8.0 | 2.0 | | 5.0 | 0.0 |
| | 3rd | 5.0 | 2.0 | | 5.7 | 0.0 |
| | Mean | 6.7 | 2.0 | #DIV/0! | 5.8 | 0.0 |
| | STDER | 0.9 | 0.0 | #DIV/0! | 0.5 | 0.0 |
| PBMC + LPS(5 ug/ml) + YDE011 1 uM | 1st | 6.0 | 2.0 | | 5.5 | 0.0 |
| | 2nd | 4.0 | 2.0 | | 4.8 | 0.0 |
| | 3rd | 6.0 | 2.0 | | 4.8 | 0.0 |
| | Mean | 5.3 | 2.0 | #DIV/0! | 5.0 | 0.0 |
| | STDER | 0.7 | 0.0 | #DIV/0! | 0.2 | 0.0 |
| PBMC + LPS(5 ug/ml) + YDE011 10 uM | 1st | 6.0 | 2.0 | | 5.4 | 0.0 |
| | 2nd | 5.0 | 2.0 | | 5.8 | 0.0 |
| | 3rd | 5.0 | 2.0 | | 4.7 | 0.0 |
| | Mean | 5.3 | 2.0 | #DIV/0! | 5.3 | 0.0 |
| | STDER | 0.3 | 0.0 | #DIV/0! | 0.3 | 0.0 |
| PBMC + LPS(5 ug/ml) + YDE011 100 uM | 1st | 4.0 | 2.0 | | 6.1 | 0.0 |
| | 2nd | 2.0 | 2.0 | | 6.1 | 0.0 |
| | 3rd | 6.0 | 2.0 | | 6.0 | 0.0 |
| | Mean | 4.0 | 2.0 | #DIV/0! | 6.1 | 0.0 |
| | STDER | 1.2 | 0.0 | #DIV/0! | 0.0 | 0.0 |
| PBMC + LPS(5 ug/ml) + YDE043 0.001 uM | 1st | 3.0 | 2.0 | | 4.4 | 0.0 |
| | 2nd | 8.0 | 2.0 | | 6.0 | 0.0 |
| | 3rd | 8.0 | 2.0 | | 4.6 | 0.0 |
| | Mean | 6.3 | 2.0 | #DIV/0! | 5.0 | 0.0 |
| | STDER | 1.7 | 0.0 | #DIV/0! | 0.5 | 0.0 |
| PBMC + LPS(5 ug/ml) + YDE043 0.01 uM | 1st | 6.0 | 2.0 | | 4.5 | 0.0 |
| | 2nd | 4.0 | 2.0 | | 6.6 | 0.0 |
| | 3rd | 4.0 | 2.0 | | 5.0 | 0.0 |
| | Mean | 4.7 | 2.0 | #DIV/0! | 5.4 | 0.0 |
| | STDER | 0.7 | 0.0 | #DIV/0! | 0.6 | 0.0 |
| PBMC + LPS(5 ug/ml) + YDE043 0.1 uM | 1st | 6.0 | 2.0 | | 5.0 | 0.0 |
| | 2nd | 3.0 | 2.0 | | 5.6 | 0.0 |
| | 3rd | 8.0 | 2.0 | | 5.6 | 0.0 |
| | Mean | 5.7 | 2.0 | #DIV/0! | 5.4 | 0.0 |
| | STDER | 1.5 | 0.0 | #DIV/0! | 0.2 | 0.0 |
| PBMC + LPS(5 ug/ml) + YDE043 1 uM | 1st | 3.0 | 2.0 | | 5.8 | 0.0 |
| | 2nd | 2.0 | 2.0 | | 4.8 | 0.0 |
| | 3rd | 4.0 | 2.0 | | 5.3 | 0.0 |
| | Mean | 3.0 | 2.0 | #DIV/0! | 5.3 | 0.0 |
| | STDER | 0.6 | 0.0 | #DIV/0! | 0.3 | 0.0 |
| PBMC + LPS(5 ug/ml) + YDE043 10 uM | 1st | 3.0 | 2.0 | | 5.2 | 0.0 |
| | 2nd | 2.0 | 2.0 | | 5.4 | 0.0 |
| | 3rd | 5.0 | 2.0 | | 5.6 | 0.0 |
| | Mean | 3.3 | 2.0 | #DIV/0! | 5.4 | 0.0 |
| | STDER | 0.9 | 0.0 | #DIV/0! | 0.1 | 0.0 |
| PBMC + LPS(5 ug/ml) + YDE043 100 uM | 1st | 2.0 | 2.0 | | 6.2 | 0.0 |
| | 2nd | 2.0 | 2.0 | | 6.4 | 0.0 |
| | Mean | 2.0 | 2.0 | #DIV/0! | 6.3 | 0.0 |
| | STDER | 0.0 | 0.0 | #DIV/0! | 0.1 | 0.0 |

TABLE 43
Bead name: IL-17A

| | | | | | | conc/ |
|---|---|---|---|---|---|---|
| | | | | Concentration | Live cell | live cell |
| Sample | | MFI | Dilution | (pg/ml) | (10\hat{ }5/ml) | (pg/10\hat{ }5) |
| Blank | 1st | 0.5 | 2.0 | NaN | | |
| | 2nd | −0.5 | 2.0 | NaN | | |
| | Mean | 0.0 | 2.0 | #DIV/0! | #DIV/0! | #DIV/0! |
| | STDER | 0.5 | 0.0 | #DIV/0! | #DIV/0! | #DIV/0! |
| PBMC + 0.5% DMSO | 1st | 0.5 | 2.0 | 1.0 | 7.5 | 0.1 |
| | 2nd | 2.5 | 2.0 | 4.3 | 7.4 | 0.6 |
| | 3rd | 1.5 | 2.0 | 2.7 | 6.6 | 0.4 |
| | Mean | 1.5 | 2.0 | 2.7 | 7.2 | 0.4 |
| | STDER | 0.6 | 0.0 | 1.0 | 0.3 | 0.1 |
| PBMC + LPS(5 ug/ml) | 1st | 4.5 | 2.0 | 7.5 | 6.1 | 1.2 |
| | 2nd | 3.5 | 2.0 | 6.0 | 5.8 | 1.0 |
| | 3rd | 4.5 | 2.0 | 7.5 | 5.5 | 1.4 |
| | Mean | 4.2 | 2.0 | 7.0 | 5.8 | 1.2 |
| | STDER | 0.3 | 0.0 | 0.5 | 0.2 | 0.1 |
| PBMC + YY101 0.01 uM | 1st | 4.5 | 2.0 | 7.5 | 5.2 | 1.5 |
| | 2nd | 4.5 | 2.0 | 7.5 | 4.8 | 1.6 |
| | Mean | 4.5 | 2.0 | 7.5 | 5.0 | 1.5 |
| | STDER | 0.0 | 0.0 | 0.0 | 0.2 | 0.1 |
| PBMC + YY101 0.1 uM | 1st | 2.5 | 2.0 | 4.3 | 4.4 | 1.0 |
| | 2nd | 7.5 | 2.0 | 12.2 | 5.8 | 2.1 |
| | Mean | 5.0 | 2.0 | 8.3 | 5.1 | 1.5 |
| | STDER | 2.5 | 0.0 | 3.9 | 0.7 | 0.6 |
| PBMC + YY101 1 uM | 1st | 3.5 | 2.0 | 6.0 | 5.5 | 1.1 |
| | 2nd | 2.5 | 2.0 | 4.3 | 5.5 | 0.8 |
| | Mean | 3.0 | 2.0 | 5.2 | 5.5 | 0.9 |
| | STDER | 0.5 | 0.0 | 0.8 | 0.0 | 0.1 |
| PBMC + YY101 10 uM | 1st | 14.5 | 2.0 | 22.9 | 6.4 | 3.6 |
| | 2nd | 8.5 | 2.0 | 13.8 | 6.1 | 2.3 |
| | Mean | 11.5 | 2.0 | 18.3 | 6.3 | 2.9 |
| | STDER | 3.0 | 0.0 | 4.6 | 0.2 | 0.7 |
| PBMC + YY101 100 uM | 1st | 1.5 | 2.0 | 2.7 | 6.4 | 0.4 |
| | 2nd | 2.5 | 2.0 | 4.3 | 7.0 | 0.6 |
| | Mean | 2.0 | 2.0 | 3.5 | 6.7 | 0.5 |
| | STDER | 0.5 | 0.0 | 0.8 | 0.3 | 0.1 |
| PBMC + LPS(5 ug/ml) + Xiidra 0.001 uM | 1st | 7.5 | 2.0 | 12.2 | 6.2 | 2.0 |
| | 2nd | 2.5 | 2.0 | 4.3 | 7.6 | 0.6 |
| | Mean | 5.0 | 2.0 | 8.3 | 6.9 | 1.3 |
| | STDER | 2.5 | 0.0 | 3.9 | 0.7 | 0.7 |
| PBMC + LPS(5 ug/ml) + Xiidra 0.01 uM | 1st | 3.5 | 2.0 | 6.0 | 5.2 | 1.1 |
| | 2nd | 3.5 | 2.0 | 6.0 | 5.8 | 1.0 |
| | Mean | 3.5 | 2.0 | 6.0 | 5.5 | 1.1 |
| | STDER | 0.0 | 0.0 | 0.0 | 0.3 | 0.1 |
| PBMC + LPS(5 ug/ml) + Xiidra 0.1 uM | 1st | 3.5 | 2.0 | 6.0 | 6.6 | 0.9 |
| | 2nd | 2.5 | 2.0 | 4.3 | 5.7 | 0.8 |
| | Mean | 3.0 | 2.0 | 5.2 | 6.2 | 0.8 |
| | STDER | 0.5 | 0.0 | 0.8 | 0.5 | 0.1 |
| PBMC + LPS(5 ug/ml) + Xiidra 1 uM | 1st | 0.5 | 2.0 | 1.0 | 4.8 | 0.2 |
| | 2nd | 1.0 | 2.0 | 1.9 | 4.4 | 0.4 |
| | Mean | 0.8 | 2.0 | 1.5 | 4.6 | 0.3 |
| | STDER | 0.3 | 0.0 | 0.4 | 0.2 | 0.1 |
| PBMC + LPS(5 ug/ml) + Xiidra 10 uM | 1st | 1.5 | 2.0 | 2.7 | 5.8 | 0.5 |
| | 2nd | 1.5 | 2.0 | 2.7 | 6.4 | 0.4 |
| | Mean | 1.5 | 2.0 | 2.7 | 6.1 | 0.4 |
| | STDER | 0.0 | 0.0 | 0.0 | 0.3 | 0.0 |
| PBMC + LPS(5 ug/ml) + Xiidra 100 uM | 1st | 1.5 | 2.0 | 2.7 | 7.1 | 0.4 |
| | 2nd | 1.5 | 2.0 | 2.7 | 5.8 | 0.5 |
| | Mean | 1.5 | 2.0 | 2.7 | 6.5 | 0.4 |
| | STDER | 0.0 | 0.0 | 0.0 | 0.7 | 0.0 |
| PBMC + LPS(5 ug/ml) + YY101 0.001 uM | 1st | 2.5 | 2.0 | 4.3 | 5.7 | 0.8 |
| | 2nd | 2.5 | 2.0 | 4.3 | 7.1 | 0.6 |
| | 3rd | 1.5 | 2.0 | 2.7 | 6.6 | 0.4 |
| | Mean | 2.2 | 2.0 | 3.8 | 6.5 | 0.6 |
| | STDER | 0.3 | 0.0 | 0.5 | 0.4 | 0.1 |
| PBMC + LPS(5 ug/ml) + YY101 0.01 uM | 1st | 3.5 | 2.0 | 6.0 | 6.4 | 0.9 |
| | 2nd | 3.5 | 2.0 | 6.0 | 6.0 | 1.0 |
| | 3rd | 1.5 | 2.0 | 2.7 | 6.1 | 0.4 |
| | Mean | 2.8 | 2.0 | 4.9 | 6.2 | 0.8 |
| | STDER | 0.7 | 0.0 | 1.1 | 0.1 | 0.2 |
| PBMC + LPS(5 ug/ml) + YY101 0.1 uM | 1st | 3.5 | 2.0 | 6.0 | 6.2 | 1.0 |
| | 2nd | 0.5 | 2.0 | 1.0 | 5.2 | 0.2 |
| | 3rd | 3.5 | 2.0 | 6.0 | 6.2 | 1.0 |
| | Mean | 2.5 | 2.0 | 4.3 | 5.9 | 0.7 |
| | STDER | 1.0 | 0.0 | 1.6 | 0.3 | 0.3 |
| PBMC + LPS(5 ug/ml) + YY101 1 uM | 1st | 2.5 | 2.0 | 4.3 | 6.3 | 0.7 |
| | 2nd | 2.5 | 2.0 | 4.3 | 7.1 | 0.6 |
| | 3rd | 4.5 | 2.0 | 7.5 | 6.2 | 1.2 |
| | Mean | 3.2 | 2.0 | 5.4 | 6.5 | 0.8 |
| | STDER | 0.7 | 0.0 | 1.1 | 0.3 | 0.2 |
| PBMC + LPS(5 ug/ml) + YY101 10 uM | 1st | 2.5 | 2.0 | 4.3 | 6.0 | 0.7 |
| | 2nd | 1.5 | 2.0 | 2.7 | 6.4 | 0.4 |
| | 3rd | 1.5 | 2.0 | 2.7 | 7.6 | 0.4 |
| | Mean | 1.8 | 2.0 | 3.3 | 6.7 | 0.5 |
| | STDER | 0.3 | 0.0 | 0.5 | 0.5 | 0.1 |
| PBMC + LPS(5 ug/ml) + YY101 100 uM | 1st | 0.5 | 2.0 | 1.0 | 6.3 | 0.2 |
| | 2nd | 1.5 | 2.0 | 2.7 | 6.2 | 0.4 |
| | 3rd | 0.5 | 2.0 | 1.0 | 5.0 | 0.2 |
| | Mean | 0.8 | 2.0 | 1.6 | 5.8 | 0.3 |
| | STDER | 0.3 | 0.0 | 0.6 | 0.4 | 0.1 |
| PBMC + LPS(5 ug/ml) + YDE011 0.001 uM | 1st | 2.5 | 2.0 | 4.3 | 4.5 | 1.0 |
| | 2nd | 1.5 | 2.0 | 2.7 | 5.8 | 0.5 |
| | 3rd | 1.5 | 2.0 | 2.7 | 5.8 | 0.5 |
| | Mean | 1.8 | 2.0 | 3.3 | 5.4 | 0.6 |
| | STDER | 0.3 | 0.0 | 0.5 | 0.4 | 0.2 |
| PBMC + LPS(5 ug/ml) + YDE011 0.01 uM | 1st | 0.5 | 2.0 | 1.0 | 6.3 | 0.2 |
| | 2nd | 0.5 | 2.0 | 1.0 | 6.2 | 0.2 |
| | 3rd | 2.5 | 2.0 | 4.3 | 5.1 | 0.9 |
| | Mean | 1.2 | 2.0 | 2.1 | 5.9 | 0.4 |
| | STDER | 0.7 | 0.0 | 1.1 | 0.4 | 0.2 |
| PBMC + LPS(5 ug/ml) + YDE011 0.1 uM | 1st | 1.5 | 2.0 | 2.7 | 6.6 | 0.4 |
| | 2nd | 0.5 | 2.0 | 1.0 | 5.0 | 0.2 |
| | 3rd | 0.5 | 2.0 | 1.0 | 5.7 | 0.2 |
| | Mean | 0.8 | 2.0 | 1.6 | 5.8 | 0.3 |
| | STDER | 0.3 | 0.0 | 0.6 | 0.5 | 0.1 |
| PBMC + LPS(5 ug/ml) + YDE011 luM | 1st | 2.5 | 2.0 | 4.3 | 5.5 | 0.8 |
| | 2nd | 2.5 | 2.0 | 4.3 | 4.8 | 0.9 |
| | 3rd | 1.5 | 2.0 | 2.7 | 4.8 | 0.6 |
| | Mean | 2.2 | 2.0 | 3.8 | 5.0 | 0.8 |
| | STDER | 0.3 | 0.0 | 0.5 | 0.2 | 0.1 |
| PBMC + LPS(5 ug/ml) + YDE011 10 uM | 1st | 1.5 | 2.0 | 2.7 | 5.4 | 0.5 |
| | 2nd | 1.5 | 2.0 | 2.7 | 5.8 | 0.5 |
| | 3rd | −0.5 | 2.0 | | 4.7 | 0.0 |
| | Mean | 0.8 | 2.0 | 2.7 | 5.3 | 0.3 |
| | STDER | 0.7 | 0.0 | 0.0 | 0.3 | 0.2 |
| PBMC + LPS(5 ug/ml) + YDE011 100 uM | 1st | 0.5 | 2.0 | 1.0 | 6.1 | 0.2 |
| | 2nd | 0.5 | 2.0 | 1.0 | 6.1 | 0.2 |
| | 3rd | −0.5 | 2.0 | | 6.0 | 0.0 |
| | Mean | 0.2 | 2.0 | 1.0 | 6.1 | 0.1 |
| | STDER | 0.3 | 0.0 | 0.0 | 0.0 | 0.1 |
| PBMC + LPS(5 ug/ml) + YDE043 0.001 uM | 1st | 0.5 | 2.0 | 1.0 | 4.4 | 0.2 |
| | 2nd | −0.5 | 2.0 | | 6.0 | 0.0 |
| | 3rd | 0.5 | 2.0 | 1.0 | 4.6 | 0.2 |
| | Mean | 0.2 | 2.0 | 1.0 | 5.0 | 0.2 |
| | STDER | 0.3 | 0.0 | 0.0 | 0.5 | 0.1 |
| PBMC + LPS(5 ug/ml) + YDE043 0.01 uM | 1st | 0.5 | 2.0 | 1.0 | 4.5 | 0.2 |
| | 2nd | 0.5 | 2.0 | 1.0 | 6.6 | 0.2 |
| | 3rd | 0.5 | 2.0 | 1.0 | 5.0 | 0.2 |
| | Mean | 0.5 | 2.0 | 1.0 | 5.4 | 0.2 |
| | STDER | 0.0 | 0.0 | 0.0 | 0.6 | 0.0 |
| PBMC + LPS(5 ug/ml) + YDE043 0.1 uM | 1st | 1.5 | 2.0 | 2.7 | 5.0 | 0.5 |
| | 2nd | 0.5 | 2.0 | 1.0 | 5.6 | 0.2 |
| | 3rd | 0.5 | 2.0 | 1.0 | 5.6 | 0.2 |
| | Mean | 0.8 | 2.0 | 1.6 | 5.4 | 0.3 |
| | STDER | 0.3 | 0.0 | 0.6 | 0.2 | 0.1 |
| PBMC + LPS(5 ug/ml) + YDE043 1 uM | 1st | 0.5 | 2.0 | 1.0 | 5.8 | 0.2 |
| | 2nd | −0.5 | 2.0 | | 4.8 | 0.0 |
| | 3rd | 1.5 | 2.0 | 2.7 | 5.3 | 0.5 |
| | Mean | 0.5 | 2.0 | 1.9 | 5.3 | 0.2 |
| | STDER | 0.6 | 0.0 | 0.8 | 0.3 | 0.2 |
| PBMC + LPS(5 ug/ml) + YDE043 10 uM | 1st | 0.0 | 2.0 | 0.1 | 5.2 | 0.0 |
| | 2nd | 1.5 | 2.0 | 2.7 | 5.4 | 0.5 |
| | 3rd | 0.5 | 2.0 | 1.0 | 5.6 | 0.2 |
| | Mean | 0.7 | 2.0 | 1.3 | 5.4 | 0.2 |
| | STDER | 0.4 | 0.0 | 0.8 | 0.1 | 0.1 |
| PBMC + LPS(5 ug/ml) + YDE043 100 uM | 1st | −0.5 | 2.0 | | 6.2 | 0.0 |
| | 2nd | −0.5 | 2.0 | | 6.4 | 0.0 |
| | Mean | −0.5 | 2.0 | #DIV/0! | 6.3 | 0.0 |
| | STDER | 0.0 | 0.0 | #DIV/0! | 0.1 | 0.0 |

TABLE 44

| | | | | | |
|---|---|---|---|---|---|
| | | | | | conc/ |
| | | | Concentration | Live cell | live cell |
| Sample | MFI | Dilution | (pg/ml) | (10\hat{ }5/ml) | (pg/10\hat{ }5) |

Bead name: IL-4

| | | | | | | |
|---|---|---|---|---|---|---|
| Blank | 1st | 0.0 | 2.0 | NaN | | |
| | 2nd | 0.0 | 2.0 | NaN | | |
| | Mean | 0.0 | 2.0 | #DIV/0! | #DIV/0! | #DIV/0! |
| | STDER | 0.0 | 0.0 | #DIV/0! | #DIV/0! | #DIV/0! |
| PBMC + 0.5% DMSO | 1st | −1.0 | 2.0 | 7.7 | 7.5 | 1.0 |
| | 2nd | −0.5 | 2.0 | 13.6 | 7.4 | 1.8 |
| | 3rd | −1.0 | 2.0 | 7.7 | 6.6 | 1.2 |
| | Mean | −0.8 | 2.0 | 9.7 | 7.2 | 1.3 |
| | STDER | 0.2 | 0.0 | 2.0 | 0.3 | 0.3 |
| PBMC + LPS(5 ug/ml) | 1st | 2.0 | 2.0 | 33.2 | 6.1 | 5.4 |
| | 2nd | 3.0 | 2.0 | 39.4 | 5.8 | 6.8 |
| | 3rd | 2.0 | 2.0 | 33.2 | 5.5 | 6.0 |
| | Mean | 2.3 | 2.0 | 35.3 | 5.8 | 6.1 |
| | STDER | 0.3 | 0.0 | 2.1 | 0.2 | 0.4 |
| PBMC + YY101 0.01 uM | 1st | 0.5 | 2.0 | 22.6 | 5.2 | 4.3 |
| | 2nd | 2.0 | 2.0 | 33.2 | 4.8 | 6.9 |
| | Mean | 1.3 | 2.0 | 27.9 | 5.0 | 5.6 |
| | STDER | 0.8 | 0.0 | 5.3 | 0.2 | 1.3 |
| PBMC + YY101 0.1 uM | 1st | 1.0 | 2.0 | 26.4 | 4.4 | 6.0 |
| | 2nd | 2.0 | 2.0 | 33.2 | 5.8 | 5.7 |
| | Mean | 1.5 | 2.0 | 29.8 | 5.1 | 5.9 |
| | STDER | 0.5 | 0.0 | 3.4 | 0.7 | 0.1 |
| PBMC + YY101 1 uM | 1st | 0.0 | 2.0 | 18.4 | 5.5 | 3.3 |
| | 2nd | 0.0 | 2.0 | 18.4 | 5.5 | 3.3 |
| | Mean | 0.0 | 2.0 | 18.4 | 5.5 | 3.3 |
| | STDER | 0.0 | 0.0 | 0.0 | 0.0 | 0.0 |
| PBMC + YY101 10 uM | 1st | 6.0 | 2.0 | 55.5 | 6.4 | 8.7 |
| | 2nd | 3.0 | 2.0 | 39.4 | 6.1 | 6.5 |
| | Mean | 4.5 | 2.0 | 47.4 | 6.3 | 7.6 |
| | STDER | 1.5 | 0.0 | 8.1 | 0.2 | 1.1 |
| PBMC + YY101 100 uM | 1st | −1.0 | 2.0 | 7.7 | 6.4 | 1.2 |
| | 2nd | 0.0 | 2.0 | 18.4 | 7.0 | 2.6 |
| | Mean | −0.5 | 2.0 | 13.0 | 6.7 | 1.9 |
| | STDER | 0.5 | 0.0 | 5.3 | 0.3 | 0.7 |
| PBMC + LPS(5 ug/ml) + Xiidra 0.001 uM | 1st | 2.0 | 2.0 | 33.2 | 6.2 | 5.4 |
| | 2nd | 2.0 | 2.0 | 33.2 | 7.6 | 4.4 |
| | Mean | 2.0 | 2.0 | 33.2 | 6.9 | 4.9 |
| | STDER | 0.0 | 0.0 | 0.0 | 0.7 | 0.5 |
| PBMC + LPS(5 ug/ml) + Xiidra 0.01 uM | 1st | 2.0 | 2.0 | 33.2 | 5.2 | 6.4 |
| | 2nd | 3.0 | 2.0 | 39.4 | 5.8 | 6.8 |
| | Mean | 2.5 | 2.0 | 36.3 | 5.5 | 6.6 |
| | STDER | 0.5 | 0.0 | 3.1 | 0.3 | 0.2 |
| PBMC + LPS(5 ug/ml) + Xiidra 0.1 uM | 1st | 1.0 | 2.0 | 26.4 | 6.6 | 4.0 |
| | 2nd | 0.0 | 2.0 | 18.4 | 5.7 | 3.2 |
| | Mean | 0.5 | 2.0 | 22.4 | 6.2 | 3.6 |
| | STDER | 0.5 | 0.0 | 4.0 | 0.5 | 0.4 |
| PBMC + LPS(5 ug/ml) + Xiidra 1 uM | 1st | 2.0 | 2.0 | 33.2 | 4.8 | 6.9 |
| | 2nd | 1.0 | 2.0 | 26.4 | 4.4 | 6.0 |
| | Mean | 1.5 | 2.0 | 29.8 | 4.6 | 6.5 |
| | STDER | 0.5 | 0.0 | 3.4 | 0.2 | 0.5 |
| PBMC + LPS(5 ug/ml) + Xiidra 10 uM | 1st | 2.0 | 2.0 | 33.2 | 5.8 | 5.7 |
| | 2nd | 1.0 | 2.0 | 26.4 | 6.4 | 4.1 |
| | Mean | 1.5 | 2.0 | 29.8 | 6.1 | 4.9 |
| | STDER | 0.5 | 0.0 | 3.4 | 0.3 | 0.8 |
| PBMC + LPS(5 ug/ml) + Xiidra 100 uM | 1st | 0.0 | 2.0 | 18.4 | 7.1 | 2.6 |
| | 2nd | 2.0 | 2.0 | 33.2 | 5.8 | 5.7 |
| | Mean | 1.0 | 2.0 | 25.8 | 6.5 | 4.2 |
| | STDER | 1.0 | 0.0 | 7.4 | 0.7 | 1.6 |
| PBMC + LPS(5 ug/ml) + YY101 0.001 uM | 1st | 0.0 | 2.0 | 18.4 | 5.7 | 3.2 |
| | 2nd | 2.0 | 2.0 | 33.2 | 7.1 | 4.7 |
| | 3rd | 1.0 | 2.0 | 26.4 | 6.6 | 4.0 |
| | Mean | 1.0 | 2.0 | 26.0 | 6.5 | 4.0 |
| | STDER | 0.6 | 0.0 | 4.3 | 0.4 | 0.4 |
| PBMC + LPS(5 ug/ml) + YY101 0.01 uM | 1st | 2.0 | 2.0 | 33.2 | 6.4 | 5.2 |
| | 2nd | 2.0 | 2.0 | 33.2 | 6.0 | 5.5 |
| | 3rd | 2.0 | 2.0 | 33.2 | 6.1 | 5.4 |
| | Mean | 2.0 | 2.0 | 33.2 | 6.2 | 5.4 |
| | STDER | 0.0 | 0.0 | 0.0 | 0.1 | 0.1 |
| PBMC + LPS(5 ug/ml) + YY101 0.1 uM | 1st | 3.0 | 2.0 | 39.4 | 6.2 | 6.4 |
| | 2nd | 4.0 | 2.0 | 45.1 | 5.2 | 8.7 |
| | 3rd | 2.0 | 2.0 | 33.2 | 6.2 | 5.4 |
| | Mean | 3.0 | 2.0 | 39.2 | 5.9 | 6.8 |
| | STDER | 0.6 | 0.0 | 3.4 | 0.3 | 1.0 |
| PBMC + LPS(5 ug/ml) + YY101 1 uM | 1st | 2.0 | 2.0 | 33.2 | 6.3 | 5.3 |
| | 2nd | 2.0 | 2.0 | 33.2 | 7.1 | 4.7 |
| | 3rd | 2.0 | 2.0 | 33.2 | 6.2 | 5.4 |
| | Mean | 2.0 | 2.0 | 33.2 | 6.5 | 5.1 |
| | STDER | 0.0 | 0.0 | 0.0 | 0.3 | 0.2 |
| PBMC + LPS(5 ug/ml) + YY101 10 uM | 1st | 1.0 | 2.0 | 26.4 | 6.0 | 4.4 |
| | 2nd | 1.0 | 2.0 | 26.4 | 6.4 | 4.1 |
| | 3rd | 0.0 | 2.0 | 18.4 | 7.6 | 2.4 |
| | Mean | 0.7 | 2.0 | 23.7 | 6.7 | 3.6 |
| | STDER | 0.3 | 0.0 | 2.7 | 0.5 | 0.6 |
| PBMC + LPS(5 ug/ml) + YY101 100 uM | 1st | 0.0 | 2.0 | 18.4 | 6.3 | 2.9 |
| | 2nd | 0.0 | 2.0 | 18.4 | 6.2 | 3.0 |
| | 3rd | −1.0 | 2.0 | 7.7 | 5.0 | 1.5 |
| | Mean | −0.3 | 2.0 | 14.8 | 5.8 | 2.5 |
| | STDER | 0.3 | 0.0 | 3.6 | 0.4 | 0.5 |
| PBMC + LPS(5 ug/ml) + YDE011 0.001 uM | 1st | 0.0 | 2.0 | 18.4 | 4.5 | 4.1 |
| | 2nd | −1.0 | 2.0 | 7.7 | 5.8 | 1.3 |
| | 3rd | −0.5 | 2.0 | 13.6 | 5.8 | 2.3 |
| | Mean | −0.5 | 2.0 | 13.2 | 5.4 | 2.6 |
| | STDER | 0.3 | 0.0 | 3.1 | 0.4 | 0.8 |
| PBMC + LPS(5 ug/ml) + YDE011 0.01 uM | 1st | 0.0 | 2.0 | 18.4 | 6.3 | 2.9 |
| | 2nd | 1.0 | 2.0 | 26.4 | 6.2 | 4.3 |
| | 3rd | 1.0 | 2.0 | 26.4 | 5.1 | 5.2 |
| | Mean | 0.7 | 2.0 | 23.7 | 5.9 | 4.1 |
| | STDER | 0.3 | 0.0 | 2.7 | 0.4 | 0.7 |
| PBMC + LPS(5 ug/ml) + YDE011 0.1 uM | 1st | 0.0 | 2.0 | 18.4 | 6.6 | 2.8 |
| | 2nd | −2.0 | 2.0 | | 5.0 | 0.0 |
| | 3rd | 1.0 | 2.0 | 26.4 | 5.7 | 4.6 |
| | Mean | −0.3 | 2.0 | 22.4 | 5.8 | 2.5 |
| | STDER | 0.9 | 0.0 | 4.0 | 0.5 | 1.3 |
| PBMC + LPS(5 ug/ml) + YDE011 1 uM | 1st | 0.0 | 2.0 | 18.4 | 5.5 | 3.3 |
| | 2nd | −1.5 | 2.0 | | 4.8 | 0.0 |
| | 3rd | 0.0 | 2.0 | 18.4 | 4.8 | 3.8 |
| | Mean | −0.5 | 2.0 | 18.4 | 5.0 | 2.4 |
| | STDER | 0.5 | 0.0 | 0.0 | 0.2 | 1.2 |
| PBMC + LPS(5 ug/ml) + YDE011 10 uM | 1st | 0.0 | 2.0 | 18.4 | 5.4 | 3.4 |
| | 2nd | −0.5 | 2.0 | 13.6 | 5.8 | 2.3 |
| | 3rd | 0.0 | 2.0 | 18.4 | 4.7 | 3.9 |
| | Mean | −0.2 | 2.0 | 16.8 | 5.3 | 3.2 |
| | STDER | 0.2 | 0.0 | 1.6 | 0.3 | 0.5 |
| PBMC + LPS(5 ug/ml) + YDE011 100 uM | 1st | −1.0 | 2.0 | 7.7 | 6.1 | 1.3 |
| | 2nd | −1.5 | 2.0 | | 6.1 | 0.0 |
| | 3rd | −1.0 | 2.0 | 7.7 | 6.0 | 1.3 |
| | Mean | −1.2 | 2.0 | 7.7 | 6.1 | 0.8 |
| | STDER | 0.2 | 0.0 | 0.0 | 0.0 | 0.4 |
| PBMC + LPS(5 ug/ml) + YDE043 0.001 uM | 1st | 1.0 | 2.0 | 26.4 | 4.4 | 6.0 |
| | 2nd | 2.0 | 2.0 | 33.2 | 6.0 | 5.5 |
| | 3rd | 0.0 | 2.0 | 18.4 | 4.6 | 4.0 |
| | Mean | 1.0 | 2.0 | 26.0 | 5.0 | 5.2 |
| | STDER | 0.6 | 0.0 | 4.3 | 0.5 | 0.6 |
| PBMC + LPS(5 ug/ml) + YDE043 0.01 uM | 1st | −1.0 | 2.0 | 7.7 | 4.5 | 1.7 |
| | 2nd | −1.0 | 2.0 | 7.7 | 6.6 | 1.2 |
| | 3rd | 0.0 | 2.0 | 18.4 | 5.0 | 3.7 |
| | Mean | −0.7 | 2.0 | 11.3 | 5.4 | 2.2 |
| | STDER | 0.3 | 0.0 | 3.6 | 0.6 | 0.8 |
| PBMC + LPS(5 ug/ml) + YDE043 0.1 uM | 1st | 1.0 | 2.0 | 26.4 | 5.0 | 5.3 |
| | 2nd | 0.0 | 2.0 | 18.4 | 5.6 | 3.3 |
| | 3rd | −1.0 | 2.0 | 7.7 | 5.6 | 1.4 |
| | Mean | 0.0 | 2.0 | 17.5 | 5.4 | 3.3 |
| | STDER | 0.6 | 0.0 | 5.4 | 0.2 | 1.1 |
| PBMC + LPS(5 ug/ml) + YDE043 1 uM | 1st | −1.5 | 2.0 | | 5.8 | 0.0 |
| | 2nd | 1.0 | 2.0 | 26.4 | 4.8 | 5.5 |
| | 3rd | 1.0 | 2.0 | 26.4 | 5.3 | 5.0 |
| | Mean | 0.2 | 2.0 | 26.4 | 5.3 | 3.5 |
| | STDER | 0.8 | 0.0 | 0.0 | 0.3 | 1.8 |
| PBMC + LPS(5 ug/ml) + YDE043 10 uM | 1st | 0.0 | 2.0 | 18.4 | 5.2 | 3.5 |
| | 2nd | −1.0 | 2.0 | 7.7 | 5.4 | 1.4 |
| | 3rd | 0.0 | 2.0 | 18.4 | 5.6 | 3.3 |
| | Mean | −0.3 | 2.0 | 14.8 | 5.4 | 2.7 |
| | STDER | 0.3 | 0.0 | 3.6 | 0.1 | 0.7 |
| PBMC + LPS(5 ug/ml) + YDE043 100 uM | 1st | −1.0 | 2.0 | 7.7 | 6.2 | 1.2 |
| | 2nd | −1.0 | 2.0 | 7.7 | 6.4 | 1.2 |
| | Mean | −1.0 | 2.0 | 7.7 | 6.3 | 1.2 |
| | STDER | 0.0 | 0.0 | 0.0 | 0.1 | 0.0 |

Bead name: VEGF-A

| | | | | | | |
|---|---|---|---|---|---|---|
| Blank | 1st | −0.3 | 2.0 | NaN | | |
| | 2nd | 0.3 | 2.0 | NaN | | |
| | Mean | 0.0 | 2.0 | #DIV/0! | #DIV/0! | #DIV/0! |
| | STDER | 0.3 | 0.0 | #DIV/0! | #DIV/0! | #DIV/0! |
| PBMC + 0.5% DMSO | 1st | 2.3 | 2.0 | 10.7 | 7.5 | 1.4 |
| | 2nd | 2.3 | 2.0 | 10.7 | 7.4 | 1.4 |
| | 3rd | 1.3 | 2.0 | 8.5 | 6.6 | 1.3 |
| | Mean | 1.9 | 2.0 | 10.0 | 7.2 | 1.4 |
| | STDER | 0.3 | 0.0 | 0.7 | 0.3 | 0.1 |
| PBMC + LPS(5 ug/ml) | 1st | 0.3 | 2.0 | 6.1 | 6.1 | 1.0 |
| | 2nd | 0.3 | 2.0 | 6.1 | 5.8 | 1.1 |
| | 3rd | −0.8 | 2.0 | 3.6 | 5.5 | 0.7 |
| | Mean | −0.1 | 2.0 | 5.3 | 5.8 | 0.9 |
| | STDER | 0.3 | 0.0 | 0.8 | 0.2 | 0.1 |
| PBMC + YY101 0.01 uM | 1st | 2.3 | 2.0 | 10.7 | 5.2 | 2.1 |
| | 2nd | 0.3 | 2.0 | 6.1 | 4.8 | 1.3 |
| | Mean | 1.3 | 2.0 | 8.4 | 5.0 | 1.7 |
| | STDER | 1.0 | 0.0 | 2.3 | 0.2 | 0.4 |
| PBMC + YY101 0.1 uM | 1st | 0.3 | 2.0 | 6.1 | 4.4 | 1.4 |
| | 2nd | 1.3 | 2.0 | 8.5 | 5.8 | 1.5 |
| | Mean | 0.8 | 2.0 | 7.3 | 5.1 | 1.4 |
| | STDER | 0.5 | 0.0 | 1.2 | 0.7 | 0.0 |
| PBMC + YY101 1 uM | 1st | 1.3 | 2.0 | 8.5 | 5.5 | 1.5 |
| | 2nd | 0.3 | 2.0 | 6.1 | 5.5 | 1.1 |
| | Mean | 0.8 | 2.0 | 7.3 | 5.5 | 1.3 |
| | STDER | 0.5 | 0.0 | 1.2 | 0.0 | 0.2 |
| PBMC + YY101 10 uM | 1st | 0.8 | 2.0 | 7.3 | 6.4 | 1.1 |
| | 2nd | 1.3 | 2.0 | 8.5 | 6.1 | 1.4 |
| | Mean | 1.0 | 2.0 | 7.9 | 6.3 | 1.3 |
| | STDER | 0.3 | 0.0 | 0.6 | 0.2 | 0.1 |
| PBMC + YY101 100 uM | 1st | −0.8 | 2.0 | 3.6 | 6.4 | 0.6 |
| | 2nd | −0.8 | 2.0 | 3.6 | 7.0 | 0.5 |
| | Mean | −0.8 | 2.0 | 3.6 | 6.7 | 0.5 |
| | STDER | 0.0 | 0.0 | 0.0 | 0.3 | 0.0 |
| PBMC + LPS(5 ug/ml) + Xiidra 0.001 uM | 1st | 0.3 | 2.0 | 6.1 | 6.2 | 1.0 |
| | 2nd | −0.8 | 2.0 | 3.6 | 7.6 | 0.5 |
| | Mean | −0.3 | 2.0 | 4.9 | 6.9 | 0.7 |
| | STDER | 0.5 | 0.0 | 1.3 | 0.7 | 0.3 |
| PBMC + LPS(5 ug/ml) + Xiidra 0.01 uM | 1st | −0.3 | 2.0 | 4.9 | 5.2 | 0.9 |
| | 2nd | −0.8 | 2.0 | 3.6 | 5.8 | 0.6 |
| | Mean | −0.5 | 2.0 | 4.2 | 5.5 | 0.8 |
| | STDER | 0.3 | 0.0 | 0.6 | 0.3 | 0.2 |
| PBMC + LPS(5 ug/ml) + Xiidra 0.1 uM | 1st | 0.3 | 2.0 | 6.1 | 6.6 | 0.9 |
| | 2nd | −2.8 | 2.0 | | 5.7 | 0.0 |
| | Mean | −1.3 | 2.0 | 6.1 | 6.2 | 0.5 |
| | STDER | 1.5 | 0.0 | #DIV/0! | 0.5 | 0.5 |
| PBMC + LPS(5 ug/ml) + Xiidra 1 uM | 1st | −0.8 | 2.0 | 3.6 | 4.8 | 0.7 |
| | 2nd | −2.3 | 2.0 | | 4.4 | 0.0 |
| | Mean | −1.5 | 2.0 | 3.6 | 4.6 | 0.4 |
| | STDER | 0.8 | 0.0 | #DIV/0! | 0.2 | 0.4 |
| PBMC + LPS(5 ug/ml) + Xiidra 10 uM | 1st | −0.8 | 2.0 | 3.6 | 5.8 | 0.6 |
| | 2nd | −1.8 | 2.0 | 0.6 | 6.4 | 0.1 |
| | Mean | −1.3 | 2.0 | 2.1 | 6.1 | 0.4 |
| | STDER | 0.5 | 0.0 | 1.5 | 0.3 | 0.3 |
| PBMC + LPS(5 ug/ml) + Xiidra 100 uM | 1st | −0.8 | 2.0 | 3.6 | 7.1 | 0.5 |
| | 2nd | −0.8 | 2.0 | 3.6 | 5.8 | 0.6 |
| | Mean | −0.8 | 2.0 | 3.6 | 6.5 | 0.6 |
| | STDER | 0.0 | 0.0 | 0.0 | 0.7 | 0.1 |
| PBMC + LPS(5 ug/ml) + YY101 0.001 uM | 1st | −1.3 | 2.0 | 2.2 | 5.7 | 0.4 |
| | 2nd | 0.3 | 2.0 | 6.1 | 7.1 | 0.9 |
| | 3rd | −1.8 | 2.0 | 0.6 | 6.6 | 0.1 |
| | Mean | −0.9 | 2.0 | 3.0 | 6.5 | 0.4 |
| | STDER | 0.6 | 0.0 | 1.6 | 0.4 | 0.2 |
| PBMC + LPS(5 ug/ml) + YY101 0.01 uM | 1st | −1.8 | 2.0 | 0.6 | 6.4 | 0.1 |
| | 2nd | −3.8 | 2.0 | | 6.0 | 0.0 |
| | 3rd | −3.8 | 2.0 | | 6.1 | 0.0 |
| | Mean | −3.1 | 2.0 | 0.6 | 6.2 | 0.0 |
| | STDER | 0.7 | 0.0 | #DIV/0! | 0.1 | 0.0 |
| PBMC + LPS(5 ug/ml) + YY101 0.1 uM | 1st | −0.8 | 2.0 | 3.6 | 6.2 | 0.6 |
| | 2nd | −1.8 | 2.0 | 0.6 | 5.2 | 0.1 |
| | 3rd | −0.8 | 2.0 | 3.6 | 6.2 | 0.6 |
| | Mean | −1.1 | 2.0 | 2.6 | 5.9 | 0.4 |
| | STDER | 0.3 | 0.0 | 1.0 | 0.3 | 0.2 |
| PBMC + LPS(5 ug/ml) + YY101 1 uM | 1st | −2.3 | 2.0 | | 6.3 | 0.0 |
| | 2nd | −1.3 | 2.0 | 2.2 | 7.1 | 0.3 |
| | 3rd | −0.8 | 2.0 | 3.6 | 6.2 | 0.6 |
| | Mean | −1.4 | 2.0 | 2.9 | 6.5 | 0.3 |
| | STDER | 0.4 | 0.0 | 0.7 | 0.3 | 0.2 |
| PBMC + LPS(5 ug/ml) + YY101 10 uM | 1st | −1.3 | 2.0 | 2.2 | 6.0 | 0.4 |
| | 2nd | −1.3 | 2.0 | 2.2 | 6.4 | 0.3 |
| | 3rd | −0.8 | 2.0 | 3.6 | 7.6 | 0.5 |
| | Mean | −1.1 | 2.0 | 2.7 | 6.7 | 0.4 |
| | STDER | 0.2 | 0.0 | 0.5 | 0.5 | 0.0 |
| PBMC + LPS(5 ug/ml) + YY101 100 uM | 1st | 0.3 | 2.0 | 6.1 | 6.3 | 1.0 |
| | 2nd | 0.3 | 2.0 | 6.1 | 6.2 | 1.0 |
| | 3rd | −1.8 | 2.0 | 0.6 | 5.0 | 0.1 |
| | Mean | −0.4 | 2.0 | 4.3 | 5.8 | 0.7 |
| | STDER | 0.7 | 0.0 | 1.8 | 0.4 | 0.3 |
| PBMC + LPS(5 ug/ml) + YDE011 0.001 uM | 1st | 0.3 | 2.0 | 6.1 | 4.5 | 1.4 |
| | 2nd | −1.8 | 2.0 | 0.6 | 5.8 | 0.1 |
| | 3rd | 0.3 | 2.0 | 6.1 | 5.8 | 1.1 |
| | Mean | −0.4 | 2.0 | 4.3 | 5.4 | 0.8 |
| | STDER | 0.7 | 0.0 | 1.8 | 0.4 | 0.4 |
| PBMC + LPS(5 ug/ml) + YDE011 0.01 uM | 1st | −0.3 | 2.0 | 4.9 | 6.3 | 0.8 |
| | 2nd | −2.8 | 2.0 | | 6.2 | 0.0 |
| | 3rd | −1.8 | 2.0 | 0.6 | 5.1 | 0.1 |
| | Mean | −1.6 | 2.0 | 2.8 | 5.9 | 0.3 |
| | STDER | 0.7 | 0.0 | 2.1 | 0.4 | 0.2 |
| PBMC + LPS(5 ug/ml) + YDE011 0.1 uM | 1st | −1.8 | 2.0 | 0.6 | 6.6 | 0.1 |
| | 2nd | −2.8 | 2.0 | | 5.0 | 0.0 |
| | 3rd | −1.8 | 2.0 | 0.6 | 5.7 | 0.1 |
| | Mean | −2.1 | 2.0 | 0.6 | 5.8 | 0.1 |
| | STDER | 0.3 | 0.0 | 0.0 | 0.5 | 0.0 |
| PBMC + LPS(5 ug/ml) + YDE011 1 uM | 1st | −0.3 | 2.0 | 4.9 | 5.5 | 0.9 |
| | 2nd | −2.8 | 2.0 | | 4.8 | 0.0 |
| | 3rd | −1.8 | 2.0 | 0.6 | 4.8 | 0.1 |
| | Mean | −1.6 | 2.0 | 2.8 | 5.0 | 0.3 |
| | STDER | 0.7 | 0.0 | 2.1 | 0.2 | 0.3 |
| PBMC + LPS(5 ug/ml) + YDE011 10 uM | 1st | −0.8 | 2.0 | 3.6 | 5.4 | 0.7 |
| | 2nd | −2.8 | 2.0 | | 5.8 | 0.0 |
| | 3rd | −1.8 | 2.0 | 0.6 | 4.7 | 0.1 |
| | Mean | −1.8 | 2.0 | 2.1 | 5.3 | 0.3 |
| | STDER | 0.6 | 0.0 | 1.5 | 0.3 | 0.2 |
| PBMC + LPS(5 ug/ml) + YDE011 100 uM | 1st | −2.8 | 2.0 | | 6.1 | 0.0 |
| | 2nd | −2.3 | 2.0 | | 6.1 | 0.0 |
| | 3rd | −2.3 | 2.0 | | 6.0 | 0.0 |
| | Mean | −2.4 | 2.0 | #DIV/0! | 6.1 | 0.0 |
| | STDER | 0.2 | 0.0 | #DIV/0! | 0.0 | 0.0 |
| PBMC + LPS(5 ug/ml) + YDE043 0.001 uM | 1st | −0.8 | 2.0 | 3.6 | 4.4 | 0.8 |
| | 2nd | −0.8 | 2.0 | 3.6 | 6.0 | 0.6 |
| | 3rd | −0.8 | 2.0 | 3.6 | 4.6 | 0.8 |
| | Mean | −0.8 | 2.0 | 3.6 | 5.0 | 0.7 |
| | STDER | 0.0 | 0.0 | 0.0 | 0.5 | 0.1 |
| PBMC + LPS(5 ug/ml) + YDE043 0.01 uM | 1st | −1.8 | 2.0 | 0.6 | 4.5 | 0.1 |
| | 2nd | −1.8 | 2.0 | 0.6 | 6.6 | 0.1 |
| | 3rd | −1.8 | 2.0 | 0.6 | 5.0 | 0.1 |
| | Mean | −1.8 | 2.0 | 0.6 | 5.4 | 0.1 |
| | STDER | 0.0 | 0.0 | 0.0 | 0.6 | 0.0 |
| PBMC + LPS(5 ug/ml) + YDE043 0.1 uM | 1st | −1.8 | 2.0 | 0.6 | 5.0 | 0.1 |
| | 2nd | −1.8 | 2.0 | 0.6 | 5.6 | 0.1 |
| | 3rd | −1.8 | 2.0 | 0.6 | 5.6 | 0.1 |
| | Mean | −1.8 | 2.0 | 0.6 | 5.4 | 0.1 |
| | STDER | 0.0 | 0.0 | 0.0 | 0.2 | 0.0 |
| PBMC + LPS(5 ug/ml) + YDE043 1 uM | 1st | 0.3 | 2.0 | 6.1 | 5.8 | 1.1 |
| | 2nd | −0.8 | 2.0 | 3.6 | 4.8 | 0.7 |
| | 3rd | −0.8 | 2.0 | 3.6 | 5.3 | 0.7 |
| | Mean | −0.4 | 2.0 | 4.4 | 5.3 | 0.8 |
| | STDER | 0.3 | 0.0 | 0.8 | 0.3 | 0.1 |
| PBMC + LPS(5 ug/ml) + YDE043 10 uM | 1st | −2.3 | 2.0 | | 5.2 | 0.0 |
| | 2nd | 0.3 | 2.0 | 6.1 | 5.4 | 1.1 |
| | 3rd | −0.3 | 2.0 | 4.9 | 5.6 | 0.9 |
| | Mean | −0.8 | 2.0 | 5.5 | 5.4 | 0.7 |
| | STDER | 0.8 | 0.0 | 0.6 | 0.1 | 0.3 |
| PBMC + LPS(5 ug/ml) + YDE043 100 uM | 1st | −0.3 | 2.0 | 4.9 | 6.2 | 0.8 |
| | 2nd | −2.8 | 2.0 | | 6.4 | 0.0 |
| | Mean | −1.5 | 2.0 | 4.9 | 6.3 | 0.4 |
| | STDER | 1.3 | 0.0 | #DIV/0! | 0.1 | 0.4 |

TABLE 45
Bead name: MMP-9

| | | | | | | conc/ |
|---|---|---|---|---|---|---|
| | | | | Concentration | Live cell | live cell |
| Sample | | MFI | Dilution | (pg/ml) | (10\hat{ }5/ml) | (pg/10\hat{ }5) |
| Blank | 1st | −0.5 | 2.0 | NaN | | |
| | 2nd | 0.5 | 2.0 | NaN | | |
| | Mean | 0.0 | 2.0 | #DIV/0! | #DIV/0! | #DIV/0! |
| | STDER | 0.5 | 0.0 | #DIV/0! | #DIV/0! | #DIV/0! |
| PBMC + 0.5% DMSO | 1st | 337.5 | 2.0 | 1614.0 | 7.5 | 215.2 |
| | 2nd | 556.5 | 2.0 | 2589.3 | 7.4 | 349.9 |
| | 3rd | 755.5 | 2.0 | 3475.0 | 6.6 | 526.5 |
| | Mean | 549.8 | 2.0 | 2559.4 | 7.2 | 363.9 |
| | STDER | 120.7 | 0.0 | 537.4 | 0.3 | 90.1 |
| PBMC + LPS(5 ug/ml) | 1st | 29.5 | 2.0 | 109.0 | 6.1 | 17.9 |
| | 2nd | 29.5 | 2.0 | 109.0 | 5.8 | 18.8 |
| | 3rd | 27.5 | 2.0 | 94.9 | 5.5 | 17.3 |
| | Mean | 28.8 | 2.0 | 104.3 | 5.8 | 18.0 |
| | STDER | 0.7 | 0.0 | 4.7 | 0.2 | 0.4 |
| PBMC + YY101 0.01 uM | 1st | 1408.5 | 2.0 | 6464.0 | 5.2 | 1243.1 |
| | 2nd | 1612.0 | 2.0 | 7431.3 | 4.8 | 1548.2 |
| | Mean | 1510.3 | 2.0 | 6947.7 | 5.0 | 1395.6 |
| | STDER | 101.8 | 0.0 | 483.7 | 0.2 | 152.6 |
| PBMC + YY101 0.1 uM | 1st | 1206.5 | 2.0 | 5521.9 | 4.4 | 1255.0 |
| | 2nd | 1844.5 | 2.0 | 8560.2 | 5.8 | 1475.9 |
| | Mean | 1525.5 | 2.0 | 7041.0 | 5.1 | 1365.4 |
| | STDER | 319.0 | 0.0 | 1519.1 | 0.7 | 110.5 |
| PBMC + YY101 1 uM | 1st | 1451.5 | 2.0 | 6666.8 | 5.5 | 1212.2 |
| | 2nd | 1598.5 | 2.0 | 7366.6 | 5.5 | 1339.4 |
| | Mean | 1525.0 | 2.0 | 7016.7 | 5.5 | 1275.8 |
| | STDER | 73.5 | 0.0 | 349.9 | 0.0 | 63.6 |
| PBMC + YY101 10 uM | 1st | 2812.5 | 2.0 | 13551.9 | 6.4 | 2117.5 |
| | 2nd | 1599.0 | 2.0 | 7369.0 | 6.1 | 1208.0 |
| | Mean | 2205.8 | 2.0 | 10460.4 | 6.3 | 1662.8 |
| | STDER | 606.8 | 0.0 | 3091.5 | 0.2 | 454.7 |
| PBMC + YY101 100 uM | 1st | 1309.5 | 2.0 | 6000.1 | 6.4 | 937.5 |
| | 2nd | 1013.0 | 2.0 | 4635.0 | 7.0 | 662.1 |
| | Mean | 1161.3 | 2.0 | 5317.6 | 6.7 | 799.8 |
| | STDER | 148.3 | 0.0 | 682.5 | 0.3 | 137.7 |
| PBMC + LPS(5 ug/ml) + Xiidra 0.001 uM | 1st | 37.5 | 2.0 | 161.5 | 6.2 | 26.1 |
| | 2nd | 38.5 | 2.0 | 167.8 | 7.6 | 22.1 |
| | Mean | 38.0 | 2.0 | 164.7 | 6.9 | 24.1 |
| | STDER | 0.5 | 0.0 | 3.1 | 0.7 | 2.0 |
| PBMC + LPS(5 ug/ml) + Xiidra 0.01 uM | 1st | 34.5 | 2.0 | 142.4 | 5.2 | 27.4 |
| | 2nd | 42.0 | 2.0 | 189.4 | 5.8 | 32.7 |
| | Mean | 38.3 | 2.0 | 165.9 | 5.5 | 30.0 |
| | STDER | 3.8 | 0.0 | 23.5 | 0.3 | 2.6 |
| PBMC + LPS(5 ug/ml) + Xiidra 0.1 uM | 1st | 28.5 | 2.0 | 102.0 | 6.6 | 15.5 |
| | 2nd | 28.5 | 2.0 | 102.0 | 5.7 | 17.9 |
| | Mean | 28.5 | 2.0 | 102.0 | 6.2 | 16.7 |
| | STDER | 0.0 | 0.0 | 0.0 | 0.5 | 1.2 |
| PBMC + LPS(5 ug/ml) + Xiidra 1 uM | 1st | 21.5 | 2.0 | 48.9 | 4.8 | 10.2 |
| | 2nd | 30.5 | 2.0 | 115.8 | 4.4 | 26.3 |
| | Mean | 26.0 | 2.0 | 82.3 | 4.6 | 18.3 |
| | STDER | 4.5 | 0.0 | 33.5 | 0.2 | 8.1 |
| PBMC + LPS(5 ug/ml) + Xiidra 10 uM | 1st | 20.5 | 2.0 | 40.2 | 5.8 | 6.9 |
| | 2nd | 21.5 | 2.0 | 48.9 | 6.4 | 7.6 |
| | Mean | 21.0 | 2.0 | 44.5 | 6.1 | 7.3 |
| | STDER | 0.5 | 0.0 | 4.3 | 0.3 | 0.4 |
| PBMC + LPS(5 ug/ml) + Xiidra 100 uM | 1st | 24.0 | 2.0 | 69.0 | 7.1 | 9.7 |
| | 2nd | 24.5 | 2.0 | 72.8 | 5.8 | 12.6 |
| | Mean | 24.3 | 2.0 | 70.9 | 6.5 | 11.1 |
| | STDER | 0.3 | 0.0 | 1.9 | 0.7 | 1.4 |
| PBMC + LPS(5 ug/ml) + YY101 0.001 uM | 1st | 36.0 | 2.0 | 152.0 | 5.7 | 26.7 |
| | 2nd | 36.5 | 2.0 | 155.2 | 7.1 | 21.9 |
| | 3rd | 50.5 | 2.0 | 239.8 | 6.6 | 36.3 |
| | Mean | 41.0 | 2.0 | 182.3 | 6.5 | 28.3 |
| | STDER | 4.8 | 0.0 | 28.7 | 0.4 | 4.3 |
| PBMC + LPS(5 ug/ml) + YY101 0.01 uM | 1st | 37.5 | 2.0 | 161.5 | 6.4 | 25.2 |
| | 2nd | 36.5 | 2.0 | 155.2 | 6.0 | 25.9 |
| | 3rd | 33.5 | 2.0 | 135.8 | 6.1 | 22.3 |
| | Mean | 35.8 | 2.0 | 150.9 | 6.2 | 24.5 |
| | STDER | 1.2 | 0.0 | 7.7 | 0.1 | 1.1 |
| | 1st | 28.5 | 2.0 | 102.0 | 6.2 | 16.5 |
| PBMC + LPS(5 ug/ml) + YY101 0.1 uM | 2nd | 31.0 | 2.0 | 119.2 | 5.2 | 22.9 |
| | 3rd | 31.5 | 2.0 | 122.6 | 6.2 | 19.8 |
| | Mean | 30.3 | 2.0 | 114.6 | 5.9 | 19.7 |
| | STDER | 0.9 | 0.0 | 6.4 | 0.3 | 1.9 |
| PBMC + LPS(5 ug/ml) + YY101 1 uM | 1st | 36.5 | 2.0 | 155.2 | 6.3 | 24.6 |
| | 2nd | 37.5 | 2.0 | 161.5 | 7.1 | 22.8 |
| | 3rd | 41.5 | 2.0 | 186.3 | 6.2 | 30.1 |
| | Mean | 38.5 | 2.0 | 167.7 | 6.5 | 25.8 |
| | STDER | 1.5 | 0.0 | 9.5 | 0.3 | 2.2 |
| PBMC + LPS(5 ug/ml) + YY101 10 uM | 1st | 37.5 | 2.0 | 161.5 | 6.0 | 26.9 |
| | 2nd | 38.5 | 2.0 | 167.8 | 6.4 | 26.2 |
| | 3rd | 34.5 | 2.0 | 142.4 | 7.6 | 18.7 |
| | Mean | 36.8 | 2.0 | 157.2 | 6.7 | 24.0 |
| | STDER | 1.2 | 0.0 | 7.7 | 0.5 | 2.6 |
| PBMC + LPS(5 ug/ml) + YY101 100 uM | 1st | 28.0 | 2.0 | 98.5 | 6.3 | 15.6 |
| | 2nd | 28.5 | 2.0 | 102.0 | 6.2 | 16.5 |
| | 3rd | 26.5 | 2.0 | 87.7 | 5.0 | 17.5 |
| | Mean | 27.7 | 2.0 | 96.1 | 5.8 | 16.5 |
| | STDER | 0.6 | 0.0 | 4.3 | 0.4 | 0.6 |
| PBMC + LPS(5 ug/ml) + YDE011 0.001 uM | 1st | 48.5 | 2.0 | 228.2 | 4.5 | 50.7 |
| | 2nd | 41.5 | 2.0 | 186.3 | 5.8 | 32.1 |
| | 3rd | 44.5 | 2.0 | 204.5 | 5.8 | 35.3 |
| | Mean | 44.8 | 2.0 | 206.3 | 5.4 | 39.4 |
| | STDER | 2.0 | 0.0 | 12.1 | 0.4 | 5.7 |
| PBMC + LPS(5 ug/ml) + YDE011 0.01 uM | 1st | 39.5 | 2.0 | 174.0 | 6.3 | 27.6 |
| | 2nd | 39.5 | 2.0 | 174.0 | 6.2 | 28.1 |
| | 3rd | 42.5 | 2.0 | 192.4 | 5.1 | 37.7 |
| | Mean | 40.5 | 2.0 | 180.2 | 5.9 | 31.1 |
| | STDER | 1.0 | 0.0 | 6.1 | 0.4 | 3.3 |
| PBMC + LPS(5 ug/ml) + YDE011 0.1 uM | 1st | 42.5 | 2.0 | 192.4 | 6.6 | 29.2 |
| | 2nd | 39.5 | 2.0 | 174.0 | 5.0 | 34.8 |
| | 3rd | 39.5 | 2.0 | 174.0 | 5.7 | 30.5 |
| | Mean | 40.5 | 2.0 | 180.2 | 5.8 | 31.5 |
| | STDER | 1.0 | 0.0 | 6.1 | 0.5 | 1.7 |
| PBMC + LPS(5 ug/ml) + YDE011 1 uM | 1st | 42.5 | 2.0 | 192.4 | 5.5 | 35.0 |
| | 2nd | 40.5 | 2.0 | 180.2 | 4.8 | 37.5 |
| | 3rd | 42.5 | 2.0 | 192.4 | 4.8 | 40.1 |
| | Mean | 41.8 | 2.0 | 188.3 | 5.0 | 37.5 |
| | STDER | 0.7 | 0.0 | 4.1 | 0.2 | 1.5 |
| PBMC + LPS(5 ug/ml) + YDE011 10 uM | 1st | 41.5 | 2.0 | 186.3 | 5.4 | 34.5 |
| | 2nd | 43.5 | 2.0 | 198.5 | 5.8 | 34.2 |
| | 3rd | 41.5 | 2.0 | 186.3 | 4.7 | 39.6 |
| | Mean | 42.2 | 2.0 | 190.4 | 5.3 | 36.1 |
| | STDER | 0.7 | 0.0 | 4.0 | 0.3 | 1.8 |
| PBMC + LPS(5 ug/ml) + YDE011 100 uM | 1st | 38.5 | 2.0 | 167.8 | 6.1 | 27.5 |
| | 2nd | 39.5 | 2.0 | 174.0 | 6.1 | 28.5 |
| | 3rd | 37.5 | 2.0 | 161.5 | 6.0 | 26.9 |
| | Mean | 38.5 | 2.0 | 167.8 | 6.1 | 27.7 |
| | STDER | 0.6 | 0.0 | 3.6 | 0.0 | 0.5 |
| PBMC + LPS(5 ug/ml) + YDE043 0.001 uM | 1st | 39.0 | 2.0 | 170.9 | 4.4 | 38.8 |
| | 2nd | 49.5 | 2.0 | 234.0 | 6.0 | 39.0 |
| | 3rd | 54.5 | 2.0 | 262.8 | 4.6 | 57.1 |
| | Mean | 47.7 | 2.0 | 222.6 | 5.0 | 45.0 |
| | STDER | 4.6 | 0.0 | 27.1 | 0.5 | 6.1 |
| PBMC + LPS(5 ug/ml) + YDE043 0.01 uM | 1st | 40.5 | 2.0 | 180.2 | 4.5 | 40.0 |
| | 2nd | 41.5 | 2.0 | 186.3 | 6.6 | 28.2 |
| | 3rd | 42.5 | 2.0 | 192.4 | 5.0 | 38.5 |
| | Mean | 41.5 | 2.0 | 186.3 | 5.4 | 35.6 |
| | STDER | 0.6 | 0.0 | 3.5 | 0.6 | 3.7 |
| PBMC + LPS(5 ug/ml) + YDE043 0.1 uM | 1st | 42.5 | 2.0 | 192.4 | 5.0 | 38.5 |
| | 2nd | 36.5 | 2.0 | 155.2 | 5.6 | 27.7 |
| | 3rd | 39.5 | 2.0 | 174.0 | 5.6 | 31.1 |
| | Mean | 39.5 | 2.0 | 173.9 | 5.4 | 32.4 |
| | STDER | 1.7 | 0.0 | 10.7 | 0.2 | 3.2 |
| PBMC + LPS(5 ug/ml) + YDE043 1 uM | 1st | 43.5 | 2.0 | 198.5 | 5.8 | 34.2 |
| | 2nd | 36.5 | 2.0 | 155.2 | 4.8 | 32.3 |
| | 3rd | 40.5 | 2.0 | 180.2 | 5.3 | 34.0 |
| | Mean | 40.2 | 2.0 | 178.0 | 5.3 | 33.5 |
| | STDER | 2.0 | 0.0 | 12.5 | 0.3 | 0.6 |
| PBMC + LPS(5 ug/ml) + YDE043 10 uM | 1st | 43.5 | 2.0 | 198.5 | 5.2 | 38.2 |
| | 2nd | 42.5 | 2.0 | 192.4 | 5.4 | 35.6 |
| | 3rd | 40.0 | 2.0 | 177.1 | 5.6 | 31.6 |
| | Mean | 42.0 | 2.0 | 189.3 | 5.4 | 35.1 |
| | STDER | 1.0 | 0.0 | 6.4 | 0.1 | 1.9 |
| PBMC + LPS(5 ug/ml) + YDE043 100 uM | 1st | 35.5 | 2.0 | 148.8 | 6.2 | 24.0 |
| | 2nd | 32.5 | 2.0 | 129.2 | 6.4 | 20.2 |
| | Mean | 34.0 | 2.0 | 139.0 | 6.3 | 22.1 |
| | STDER | 1.5 | 0.0 | 9.8 | 0.1 | 1.9 |

### Example 10: Establish Effects of YY-101, YDE-011, YDE-043, YDE-048 and YDE-060 Compounds on 25 Selected Cytokine and Chemokine Release in Tears from Rats with ELGE Dry Eye Condition

### Objective:

To establish effects of YY-101, YDE-011, YDE-043, YDE-048 and YDE-060 compounds on 25 selected cytokine and chemokine release in tears from rats with ELGE dry eye condition.

### Materials:

Rat IL-6 ELISA kit (Thermo fisher, USA)
Rat magnetic Luminex assay kit—Procartaplex (Thermo fisher, USA)

### Reference Compound Used:

Xiidra® (Shire, USA)

### Test Compound

0.3% YY-101, 0.3% YDE-011, 0.3% YDE-043, 0.3% YDE-048 and 0.3% YDE-060.

### Summary of the Protocol

1. Rats with ELGE were treated with YY & YDE compound in 5 ul eye drops twice a day for 2 weeks.
2. Tear samples were collected using a capillary tube and stored in −80° C. freezer until analysis.
3. Tear samples were combined within the group and protein were quantified using the BCA assay.
4. Cytokines level is assessed by multiplex assay following manufacturer's instructions.

### Statistical Analysis

All values are presented as mean±standard deviation (SD).

### Results

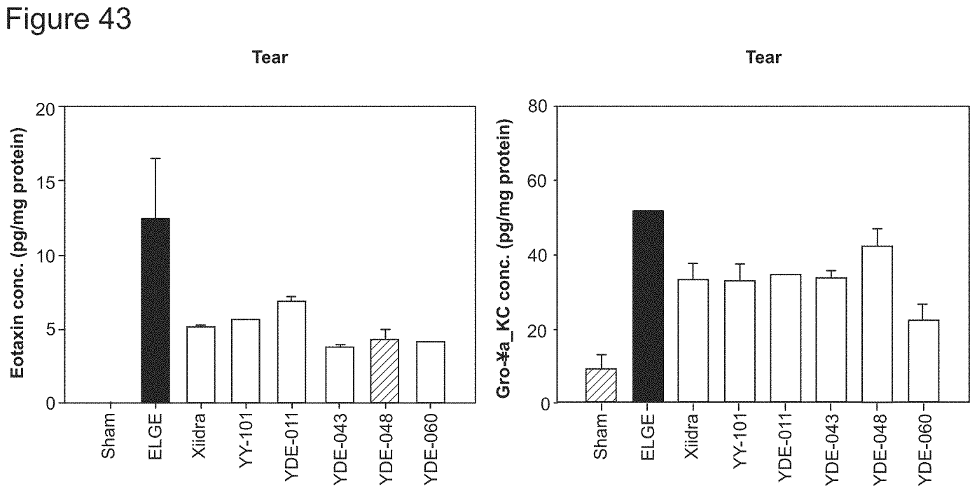

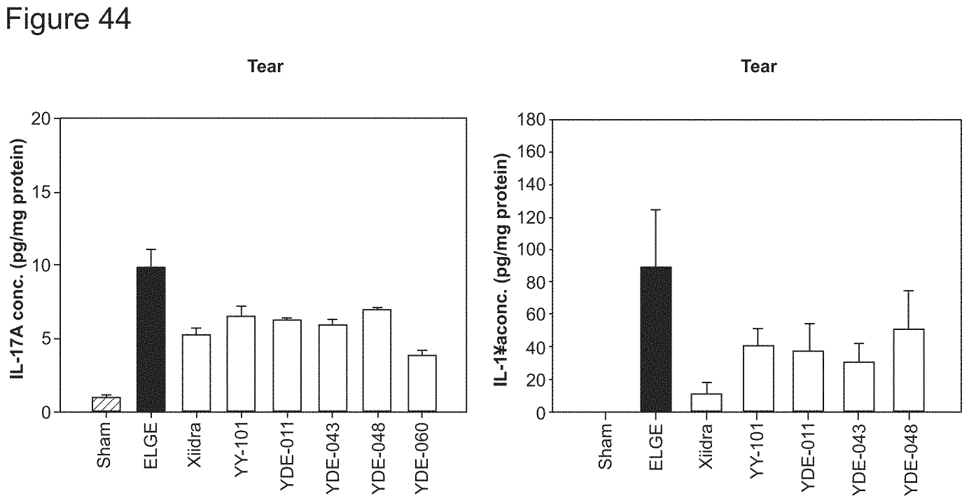

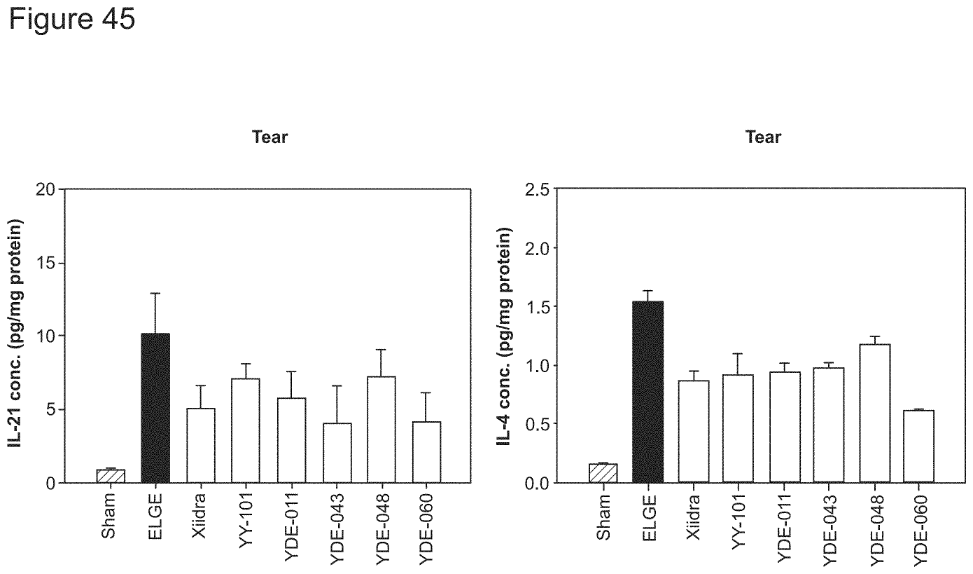

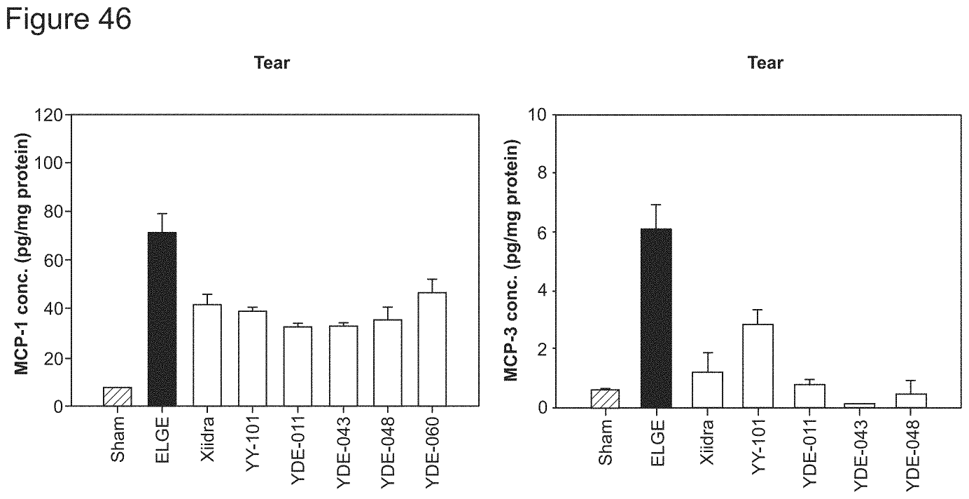

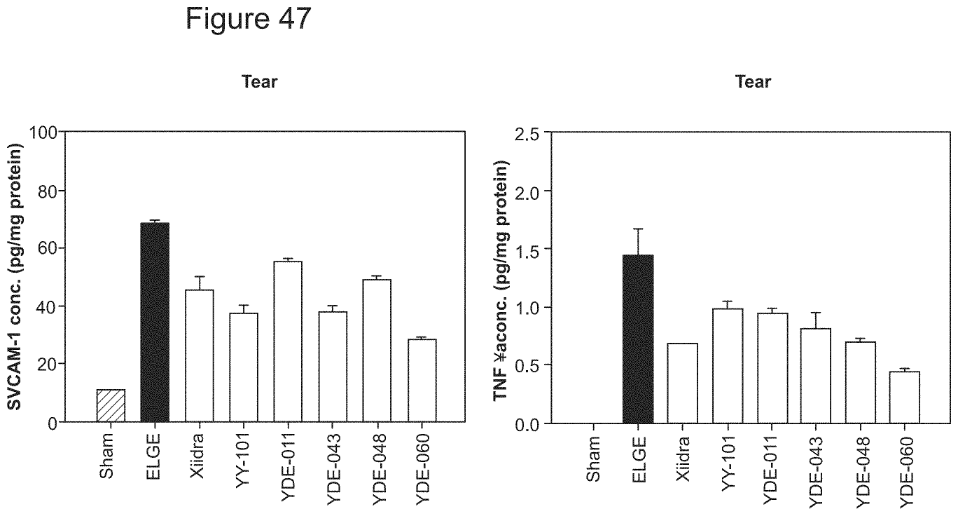

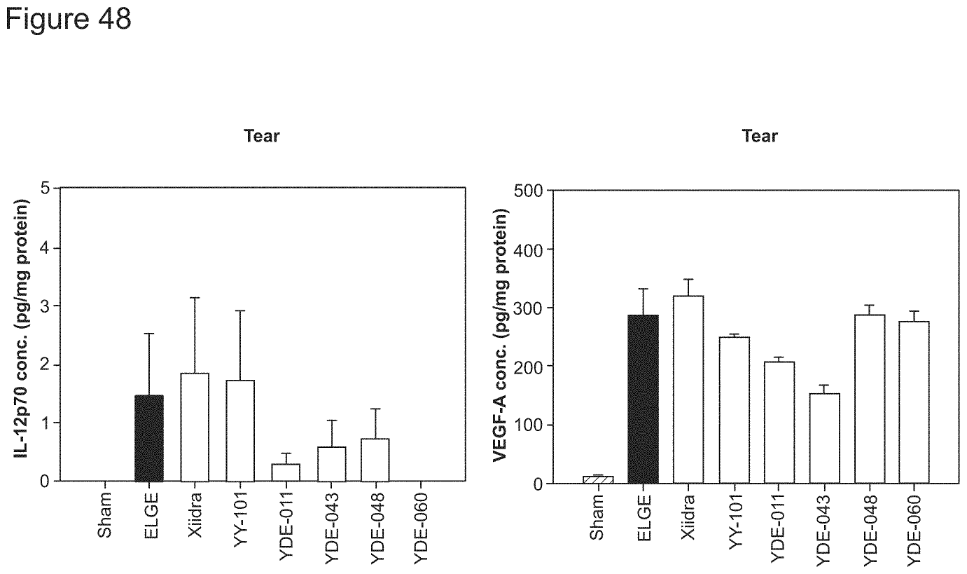

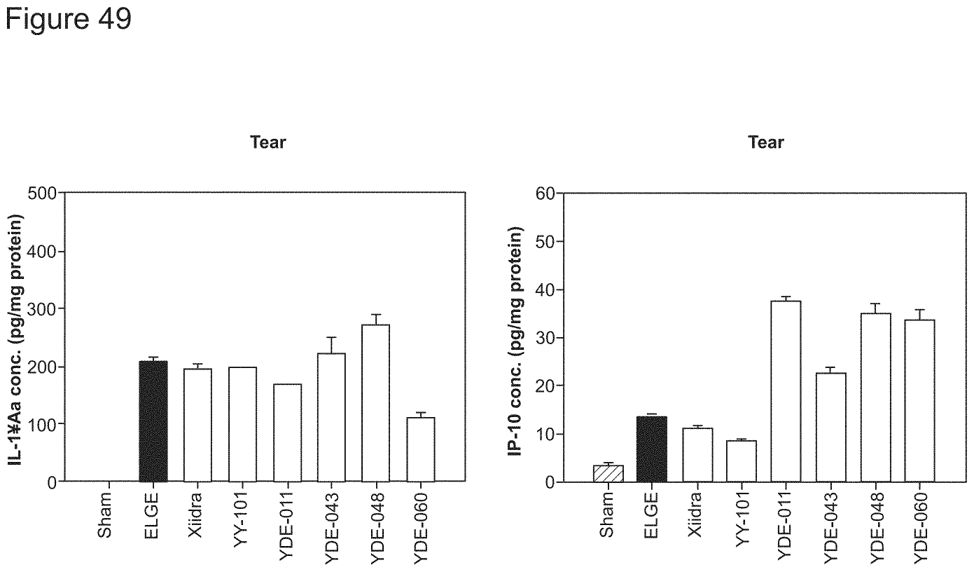

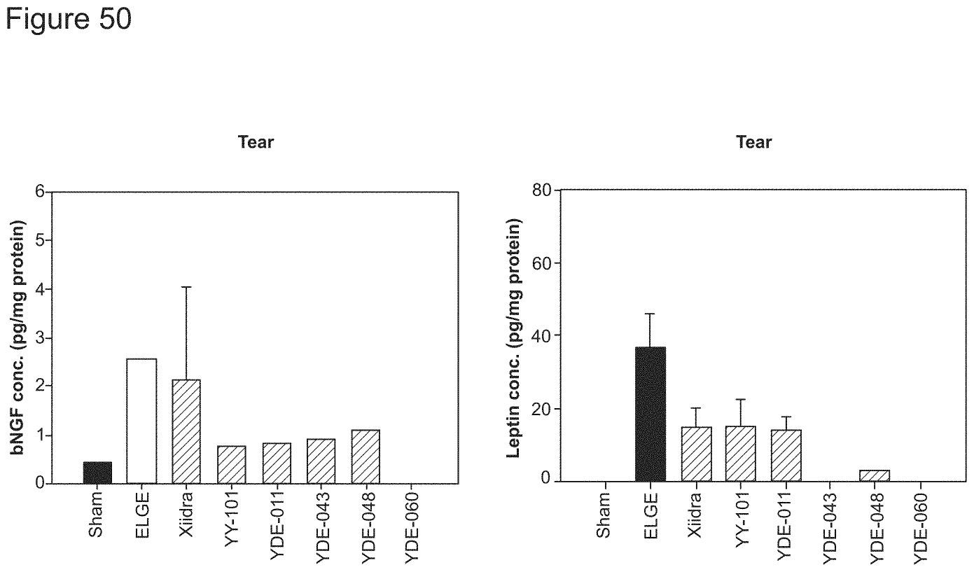

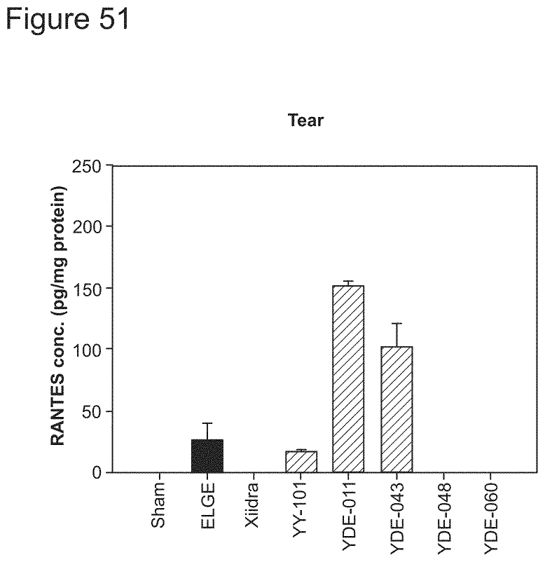

The results are shown in FIGS. 43-51 and are presented as concentration (pg/mg protein). All data was normalized to their protein levels. Out of 25 cytokines & chemokines evaluated, 14 of them were within the standard curve range, 3 were below the range, 1 over the range and 7 were below the level of detection & quantification. Since the amount of sample was very small (2-10 uL), each dilution factor was selected in order to generate min. volume for Luminex measurement.

TABLE 46

| No. | Group | Dilution factor |
|---|---|---|
| 1 | Sham | 1/12 |
| 2 | ELGE | 1/12 |
| 3 | Xiidra | 1/12 |
| 4 | YY-101 | 1/12 |
| 5 | YDE-011 | 1/14/75 |
| 6 | YDE-043 | 1/12 |
| S | YDE-048 | 1/19/33 |
| 8 | YDE-060 | 1/12 |

TABLE 47

| No. | Cytokine | Result |
|---|---|---|
| 1 | Eotaxin | Within standard curve range |
| 2 | Gro-α_KC | Within standard curve range |
| 3 | IL-17A | Within standard curve range |
| 4 | IL-1 13 | Within standard curve range |
| 5 | IL-21 | Within standard curve range |
| 6 | IL-4 | Within standard curve range |
| 7 | MCP-1 | Within standard curve range |
| 8 | MCP-3 | Within standard curve range |
| 9 | sVCAM-1 | Within standard curve range |
| 10 | TNF-α | Within standard curve range |
| 11 | IL-12p70 | Within standard curve range |
| 12 | IL-1 α | Within standard curve range |
| 13 | IP-10 | Within standard curve range |
| 14 | VEGF-A | Within standard curve range |
| 15 | bNGF | Below standard curve range |
| 16 | Leptin | Below standard curve range |
| 17 | RANTES | Below standard curve range |
| 18 | CRP | OLQ |
| 19 | TGF-13 | BLQ |
| 20 | IFN-y | BLQ |
| 21 | IL-10 | BLQ |
| 22 | IL-2 | BLQ |
| 23 | IL-6 | BLQ |
| 24 | MIP-1 α | BLQ |
| 25 | sRANKL | BLQ |

Key:
OLQ = Over the level of quantification
BLQ = Below the level of Quantification

### Summary & Conclusion

Due to small samples of tears, all individual samples were pooled within the groups, thus, the statistical analysis was not performed. Despite of the small sample amount, most of selected cytokine and chemokine levels in the conjunctival epithelial cells were detected within the normal standard range.
As expected, a 2 week ELGE induced dry eye condition in rats led to a significant increase of proinflammatory cytokine/chemokine levels in tears of ELGE animals compared to those in the sham control groups.
Both the test compounds and the reference compound, Xiidra, significantly reduced most of Th1, Th2 & Th17 derived cytokines and proinflammatory chemokines, suggesting that improvement of corneal damages in ELGE rat dry condition is, at least partly, due to the effective treatment in reducing the proinflammatory cytokine and chemokines in the eye.

### Example 11: Establish Effects of YDE-048 and YDE-043 Compounds on 25 Selected Cytokine and Chemokine Release from Conjunctival Epithelial Cells (Including Goblet Cells) of Rats with ELGE Dry Eye Condition

### Objective

To establish effects of YDE-048 and YDE-043 compounds on 25 selected cytokine and chemokine release from conjunctival epithelial cells (including goblet cells) of rats with ELGE dry eye condition (from NSY0319 study).

### Materials

Rat IL-6 ELISA kit (Thermo fisher, USA, cat no: BMS625)
Rat magnetic Luminex assay kit—Procartaplex (Thermo fisher, USA, cat no: PPX-25-MX9HJJU)

### Test Compound

0.1%, 0.3%, 1%, 3% of YDE-048 & 1% of YDE-043.

### Statistical Analysis

All values are presented as mean±standard deviation (SD).
All values were statistically analyzed by one-way ANOVA with a Tukey, Dunnett's post test comparing all the groups.

### Results

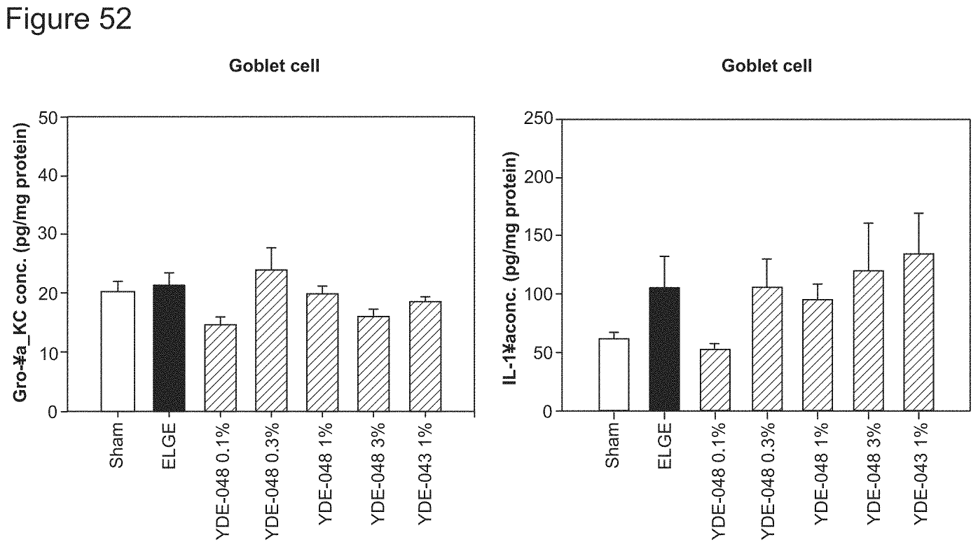

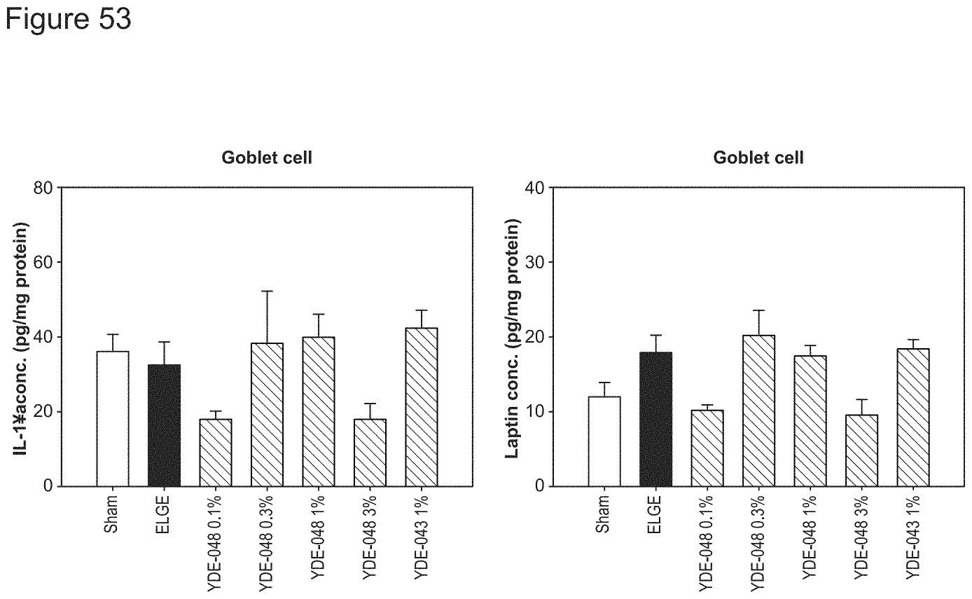

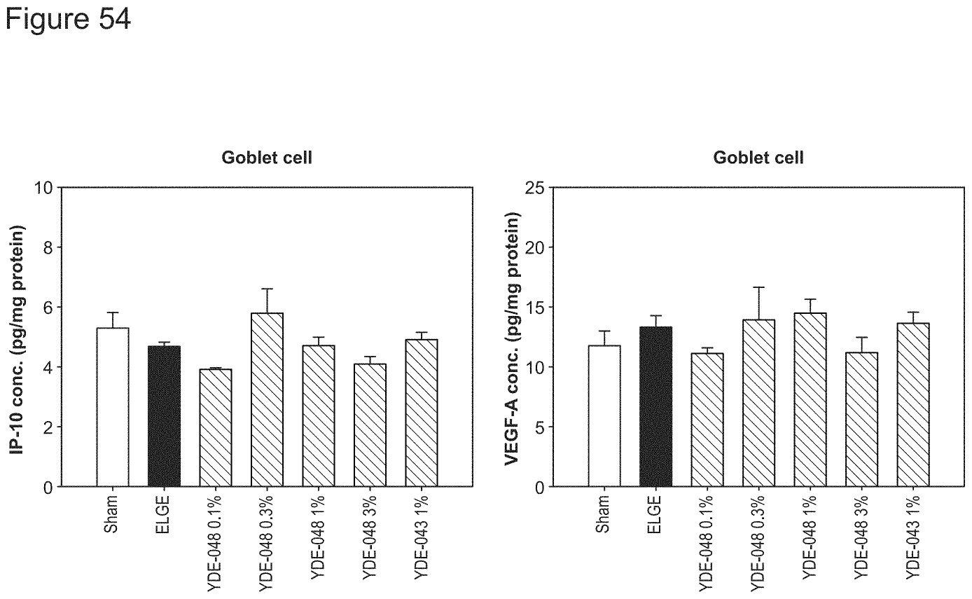

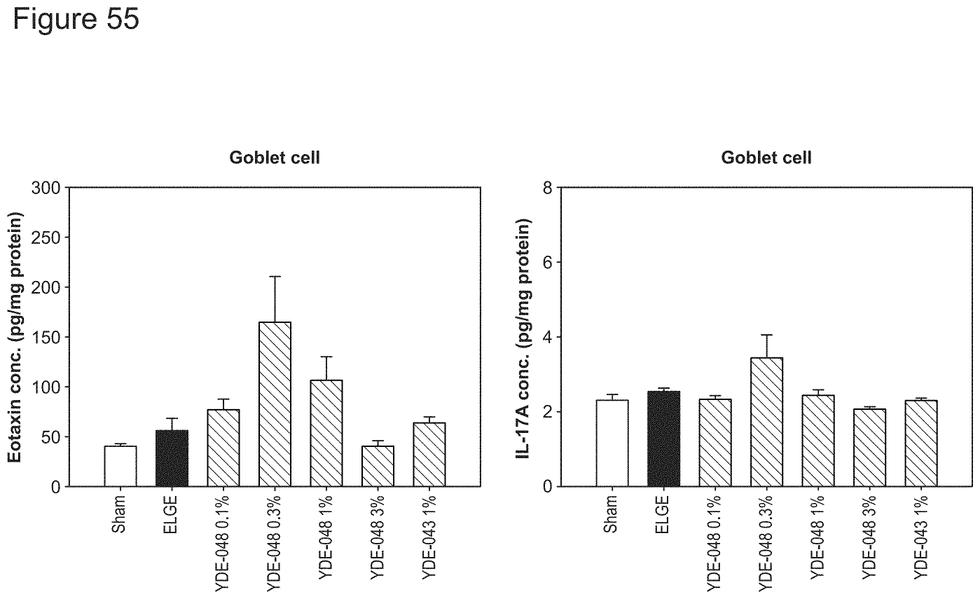

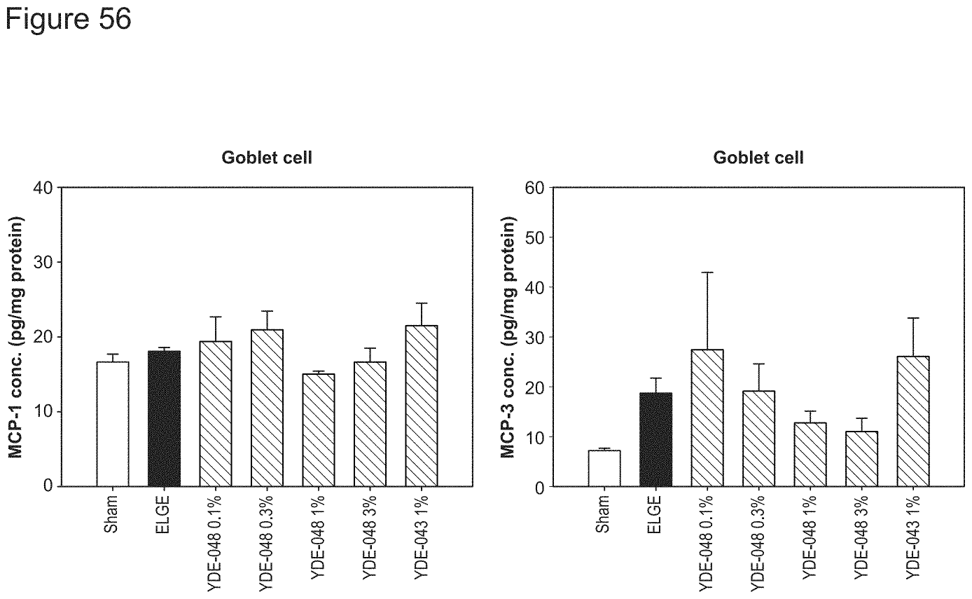

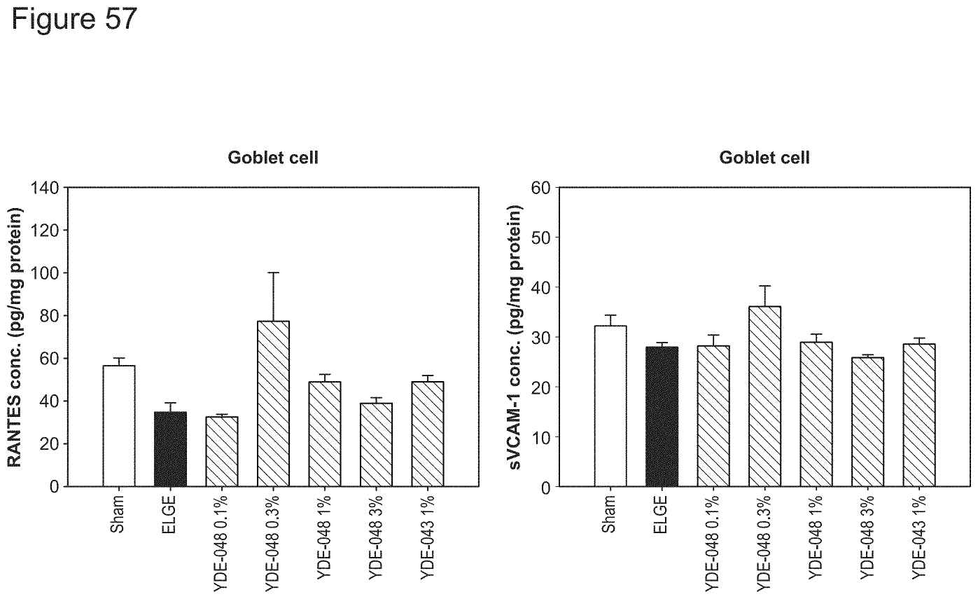

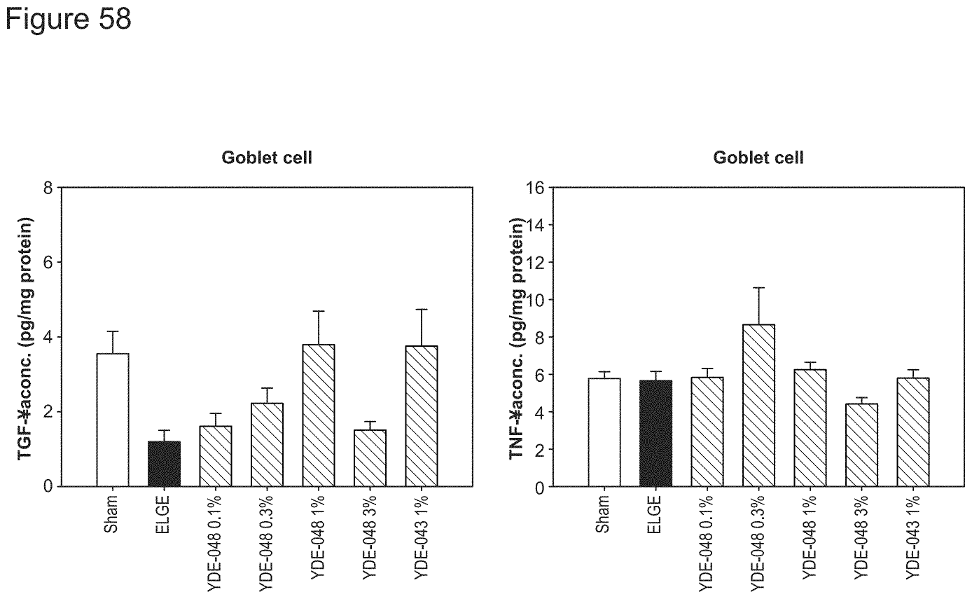

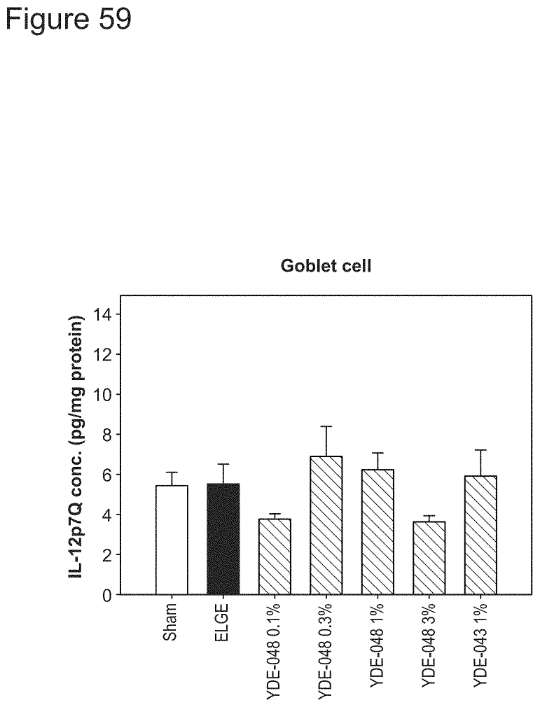

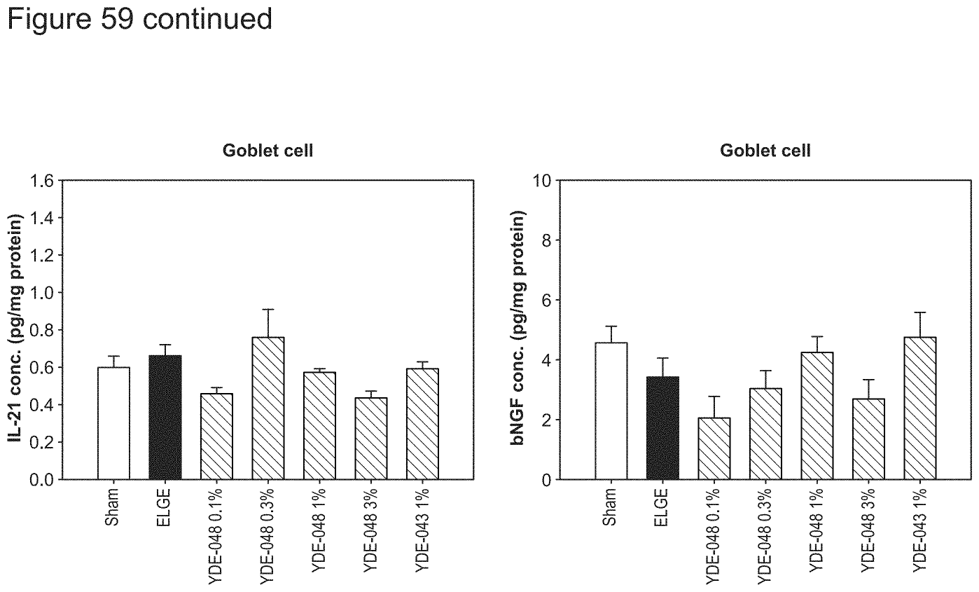

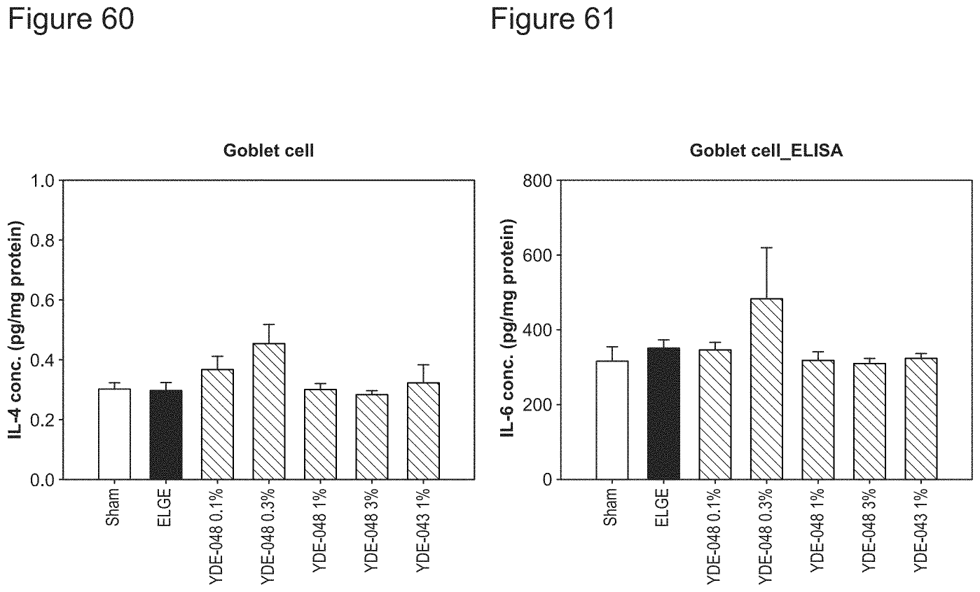

The results are shown in FIGS. 52-61 and are presented as concentration (pg/mg protein). All data was normalized to their protein levels. Out of 25 cytokines & chemokines evaluated, 14 of them were within the standard curve range, 4 were below the range, 1 over the range and 6 were below the level of detection & quantification.

TABLE 48

| | | Dilution | |
|---|---|---|---|
| No. | Cytokine | factor | Result |
| 1 | Gro-α_KC | 1:01 | Within standard curve range |
| 2 | IL-1 α | 1:01 | Within standard curve range |
| 3 | IL-1 13 | 1:01 | Within standard curve range |
| 4 | Leptin | 1:01 | Within standard curve range |
| 5 | IP-10 | 1:01 | Within standard curve range |
| 6 | VEGF-A | 1:01 | Within standard curve range |
| 7 | Eotaxin | 1:01 | Within standard curve range |
| 8 | IL-17A | 1:01 | Within standard curve range |
| 9 | MCP-1 | 1:01 | Within standard curve range |
| 10 | MCP-3 | 1:01 | Within standard curve range |
| 11 | RANTES | 1:01 | Within standard curve range |
| 12 | sVCAM-1 | 1:01 | Within standard curve range |
| 13 | TGF-13 | 1:01 | Within standard curve range |
| 14 | TNF-α | 1:01 | Within standard curve range |
| 15 | IL-12p70 | 1:01 | Below standard curve range |
| 16 | IL-21 | 1:01 | Below standard curve range |
| 17 | bNGF | 1:01 | Below standard curve range |
| 18 | IL-4 | 1:01 | Below standard curve range |
| 19 | CRP | 1:01 | OLQ |
| 20 | IFN-γ | 1:01 | BLQ |
| 21 | IL-10 | 1:01 | BLQ |
| 22 | IL-2 | 1:01 | BLQ |
| 23 | IL-6 | 1:01 | BLQ |
| 24 | MIP-1 α | 1:01 | BLQ |
| 25 | sRANKL | 1:01 | BLQ |

Key:
OLQ = Over the level of quantification
BLQ = Below the level of Quantification

### Summary & Conclusion

Most of selected cytokine and chemokine levels in the conjunctival epithelial cells were detected within the normal standard range.
Literature suggests that the dry eye condition leads to increase the level of proinflammatory cytokines (e.g., TNF-alpha, IL-6, IL-17) in the conjunctival epithelial cells dry eye disease. However, a 2 week ELGE induced dry eye condition in rats didn't elicit proinflammatory cytokine/chemokine induction in conjunctival epithelial cells in ELGE animals compared to those in the sham control groups.
None of the compounds significantly affected cytokine/chemokine production, suggesting that the compounds have little or no effect the basal level of proinflammatory cytokine/chemokines (including IL-6 levels) in conjunctival epithelial cells although YDE-048 and YDE-043 were effective in improving corneal damaged induced by ELGE.

### Example 12: Efficacy Evaluation of the Compounds of the Invention in Mouse IBD Model

### DSS Induced Colitis:

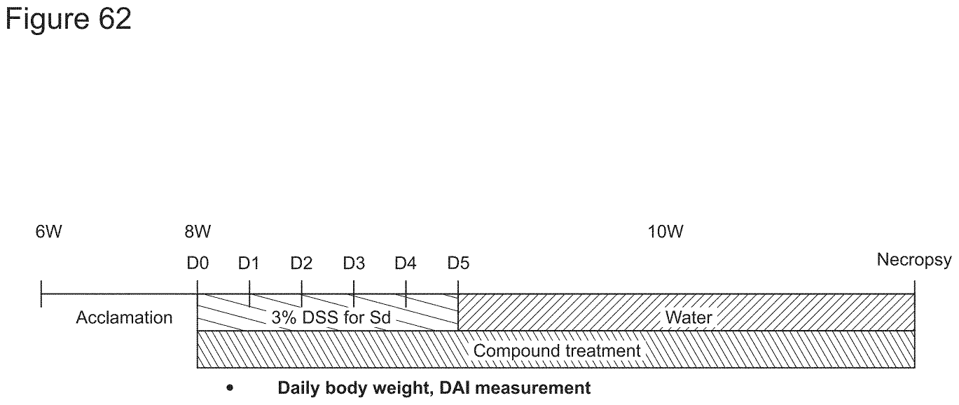

Experimental schematic timeline is shown in FIG. 62.

TABLE 49

| | | | |
|---|---|---|---|
| Group | mpk | N | Route |

| | | | | | |
|---|---|---|---|---|---|
| 1 | Control (No DSS) | | 6 + 2 | SC | B.I.D |
| 2 | DSS | | 6 + 2 | SC | |
| 3 | DSS + Cyclosporin A | 20 | 6 + 2 | PO | |
| 4 | DSS + YDE-048 | 10 | 6 + 2 | SC | |
| 5 | DSS + YDE-048 | 100 | 6 + 2 | SC | |

YDE-048 is soluble in saline

End-point termination—tissue and handling hours after last dosing, collect terminal blood sample, colon
Blood chemistry evaluation (i.e., CRP)
Histology and pathology evaluation—H&E
Inflammation and oxidative markers (e.g, TNF-α, nitrotyrosine)

TABLE 50
Body weight (BW) loss (%) score:

| | |
|---|---|
| 0-1% | =0 |
| 1-5% | =1 |
| 5-10% | =2 |
| 10-20% | =3 |
| 20% | =4 |

Stool consistency (SC) score:

| | |
|---|---|
| Normal | =0 |
| Soft, but still formed | =1 |
| Very soft | =2 |
| Diarrhea | =4 |

Fecal blood occurrence (FB) score:

| | |
|---|---|
| Normal | =0 |
| Visible traces of blood - slight | =1 |
| Visible traces of blood - critical | =2 |
| Gross rectal bleeding | =4 |
| Disease Activity Index (DAI) | =BW score + SC score + FB score |

TABLE 51

| Group | | Mpk | N |
|---|---|---|---|
| G1 | Control (No DSS) | | 4 |
| G2 | DSS | | 8 |
| G3 | DSS + Cyclosporin A | 20 | 8 |
| G4 | DSS + YDE-048 | 10 | 5 |
| G5 | DSS + YDE-048 | 100 | 8 |

### DSS Induced Colitis

Histology pathology evaluation—H&E
Inflammation and oxidative markers—TNF-α, Nitrotyrosine
   TNF-α: 3 image (different 3 layer of on direction) per sample (area: Intestinal villus)
   Nitrotyrosine: 2 image (including all layer in Right, Left) per sample

### Summary

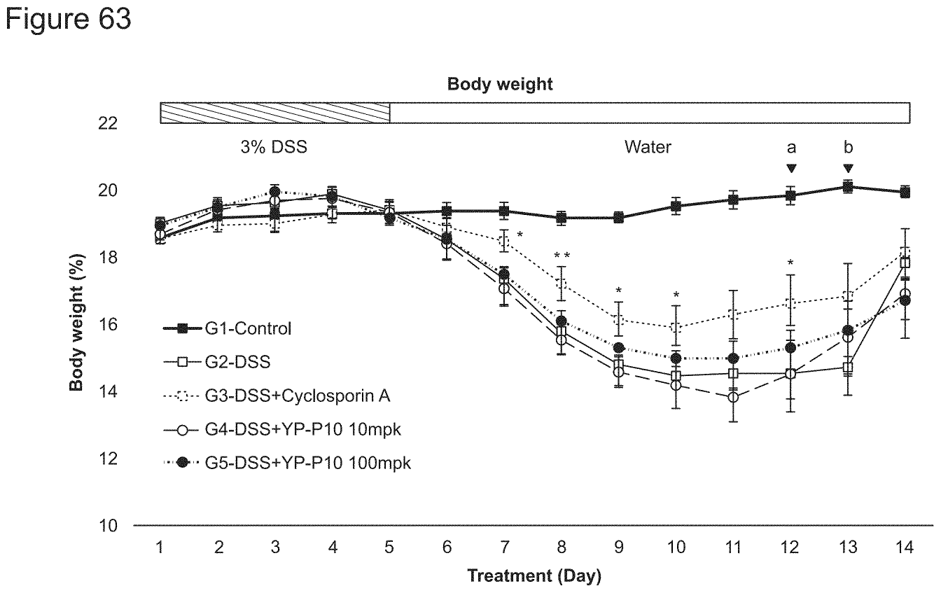

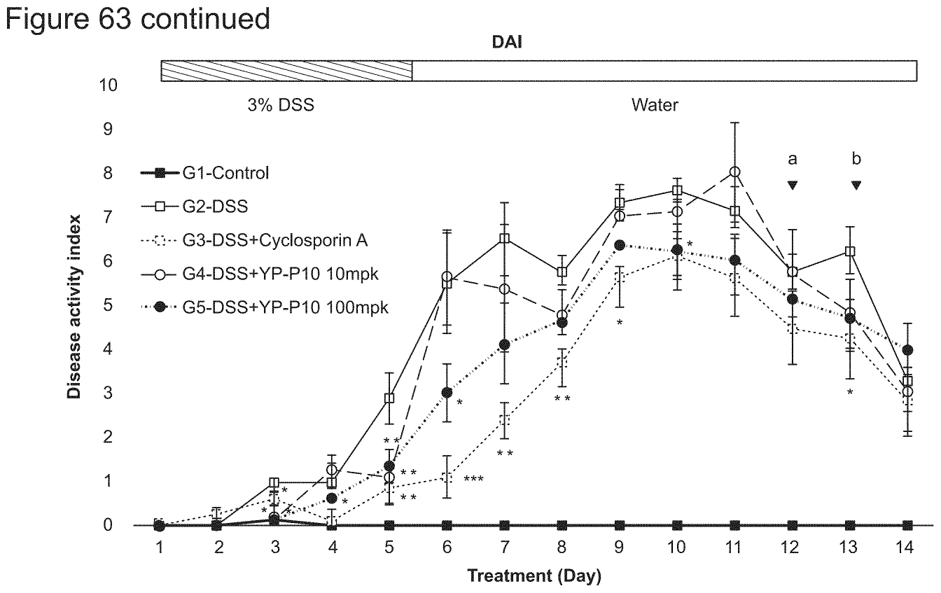

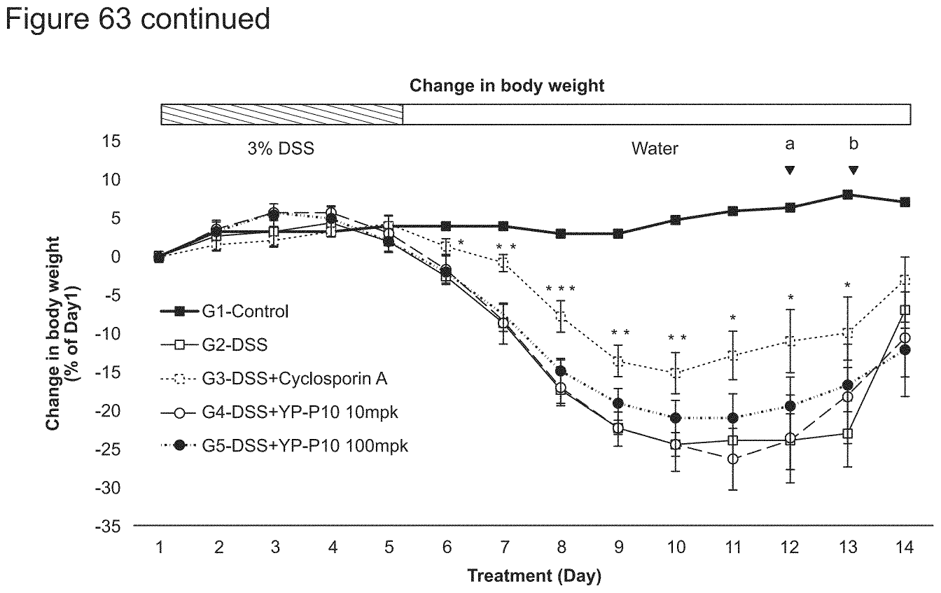

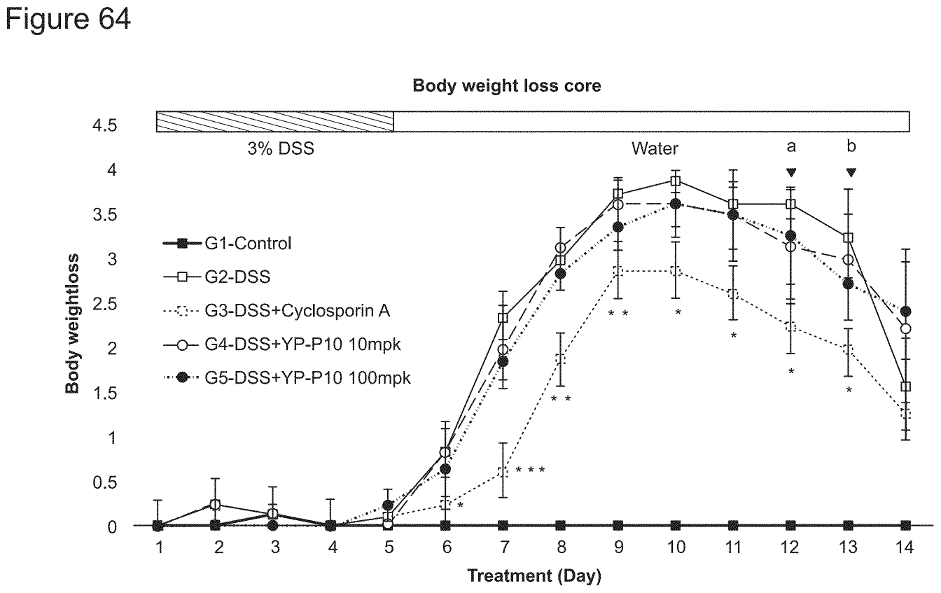

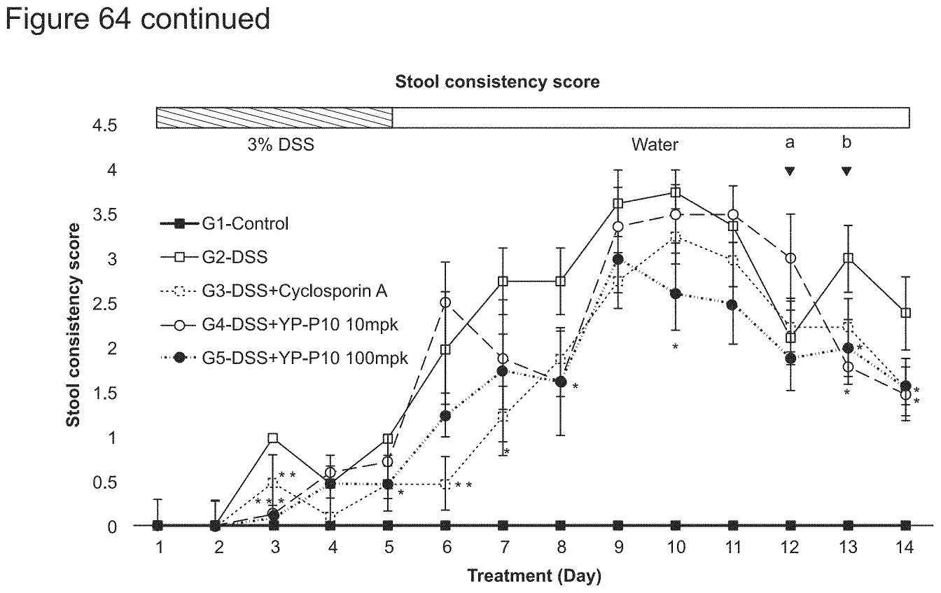

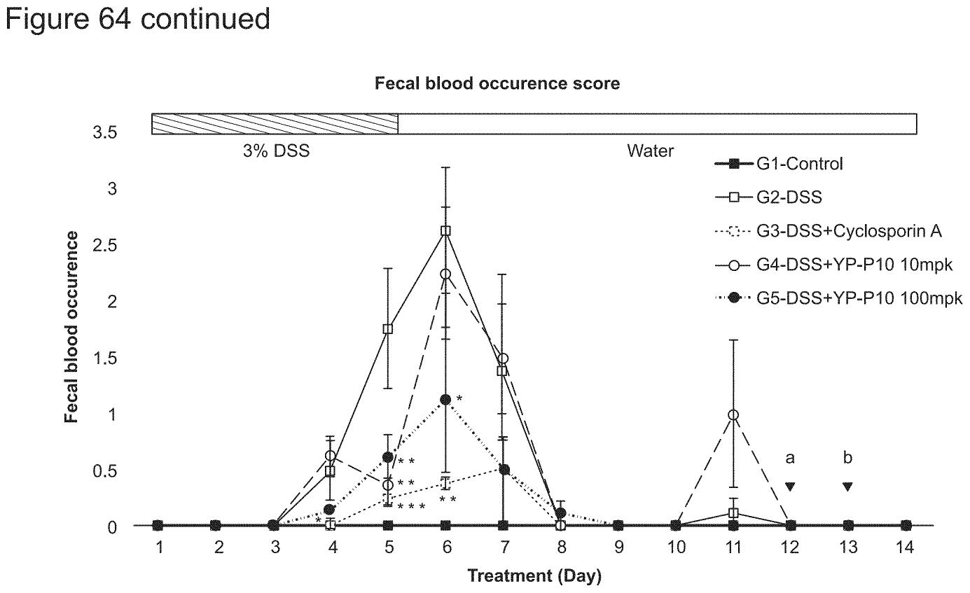

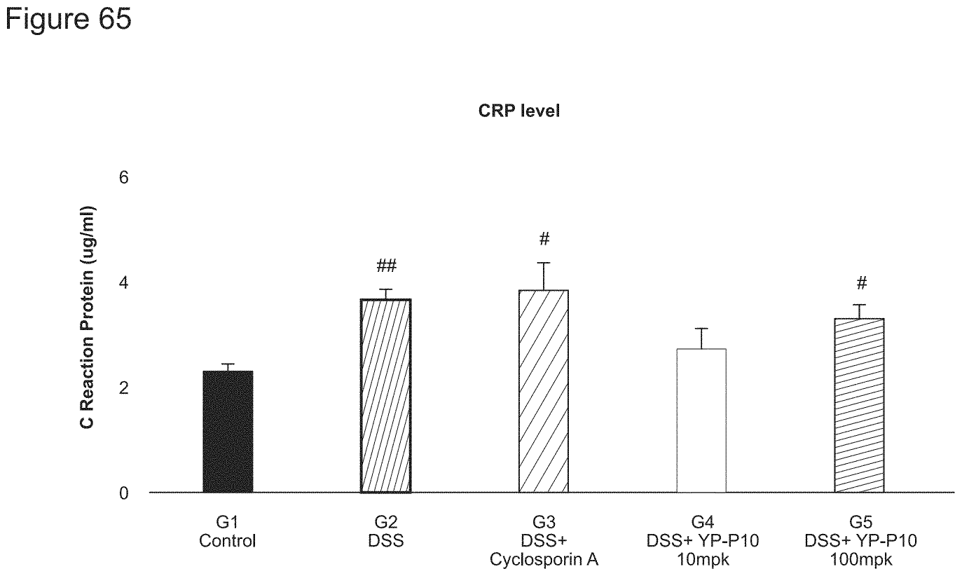

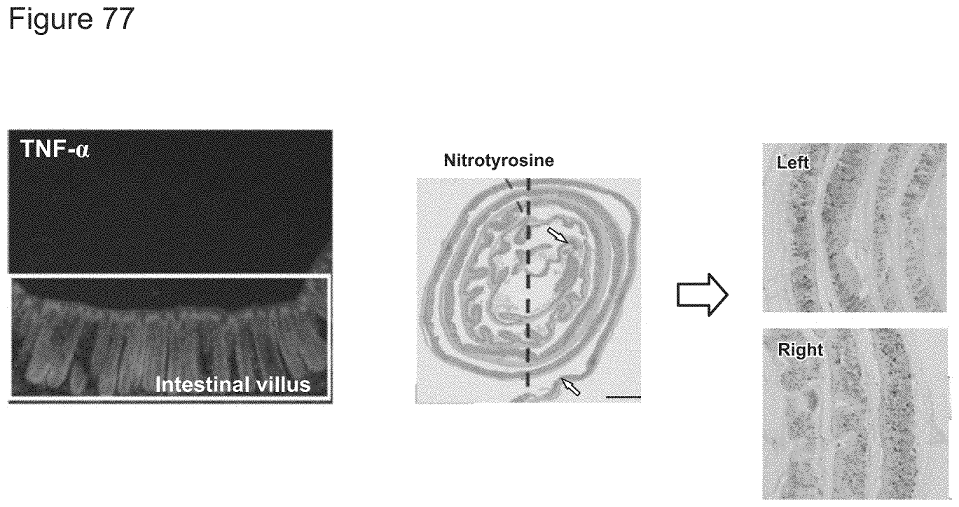

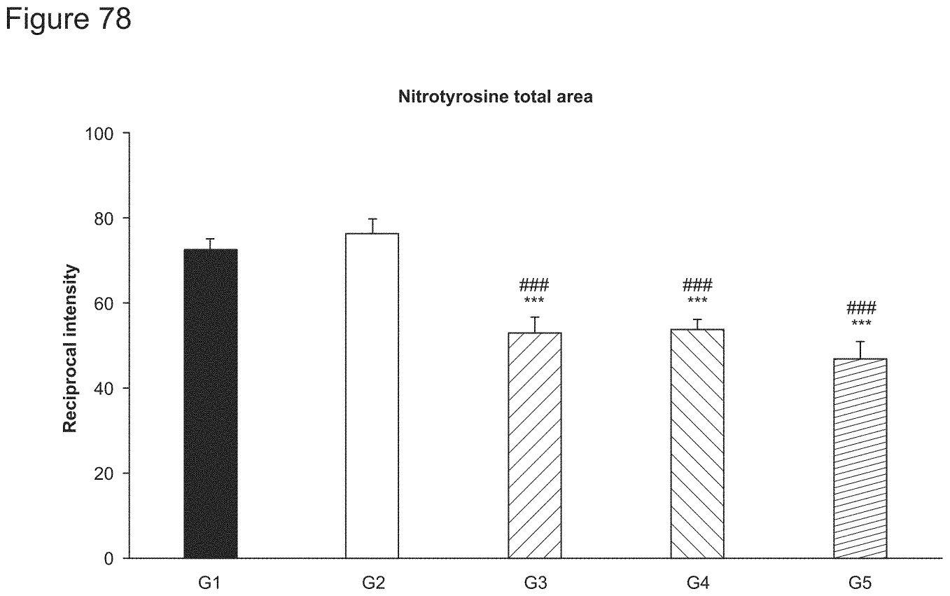

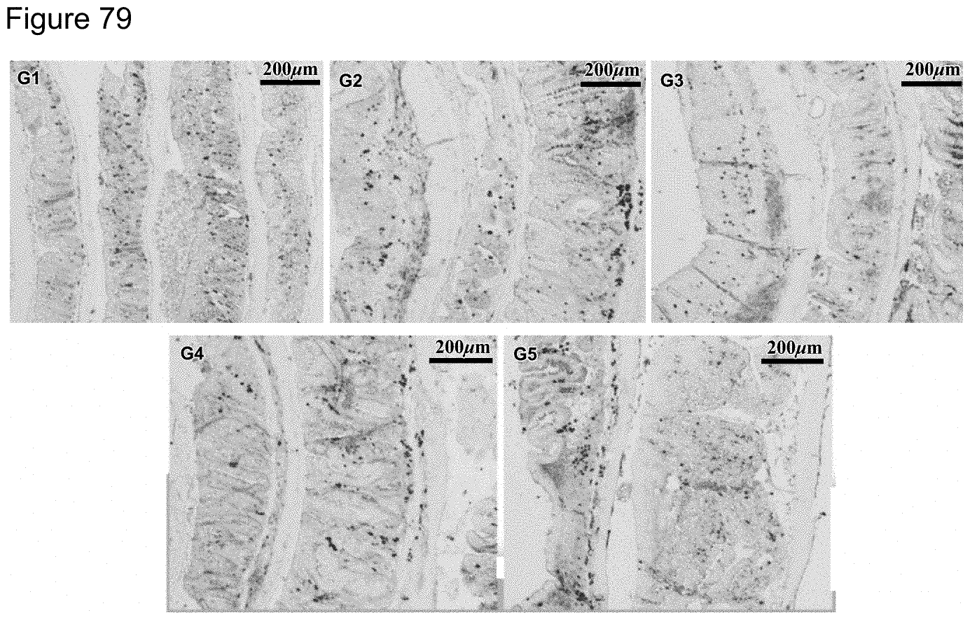

3% DSS for five days induced body weight loss, increased disease activity index (DSI) in mice
Increased disease index includes body weight loss and increased appearance of loose stools and fecal blood
A positive reference compound, cyclosporin A, significantly reduced DSS-induced body weight and increased DSI.
YDE-048 at 100 mg/kg significantly reduced DSS-induced increased DSI. In particular, YDE-048 reduced DSS-induced occurrence of loose stools and fecal blood.
The level of C-reactive protein (pro-inflammation marker) is highly elevated in plasma of mice with IBD, and YDE-048 partially reduced the level.
Future studies will focus on increased sample size, higher dose (e.g., 300 mg/kg) and/or alternative drug delivery (Alzet pump or Matrigel)
   Immunohistological evaluation on intestines show that TNF-α level is significantly elevated in mice with IBD, and YDE-048 significantly reduced both TNF-α (pro-inflammatory cytokine) and nitrotyrosine (a marker for protein oxidation) levels.
   The data and results are shown in FIGS. 62-65 and FIGS. 77-79.

### Example 13: Mouse Rheumatoid Arthritis Model

### Summary:

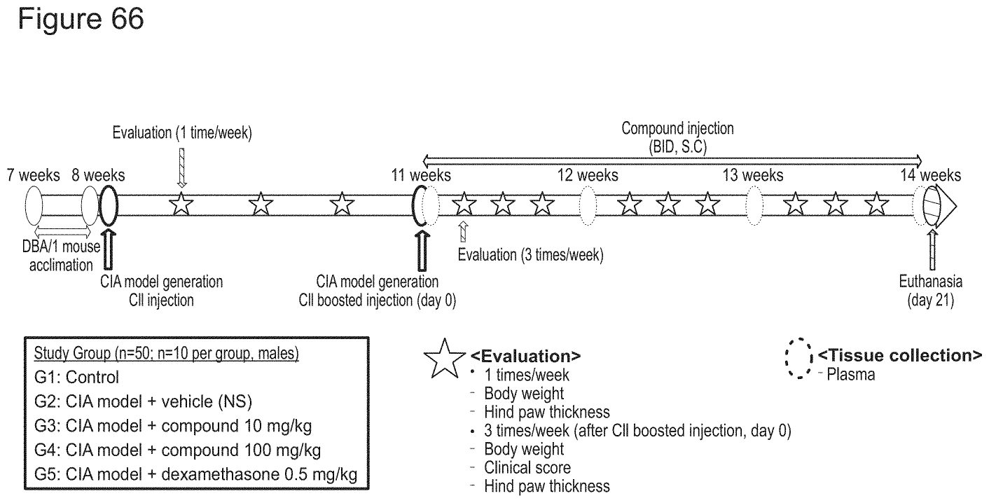

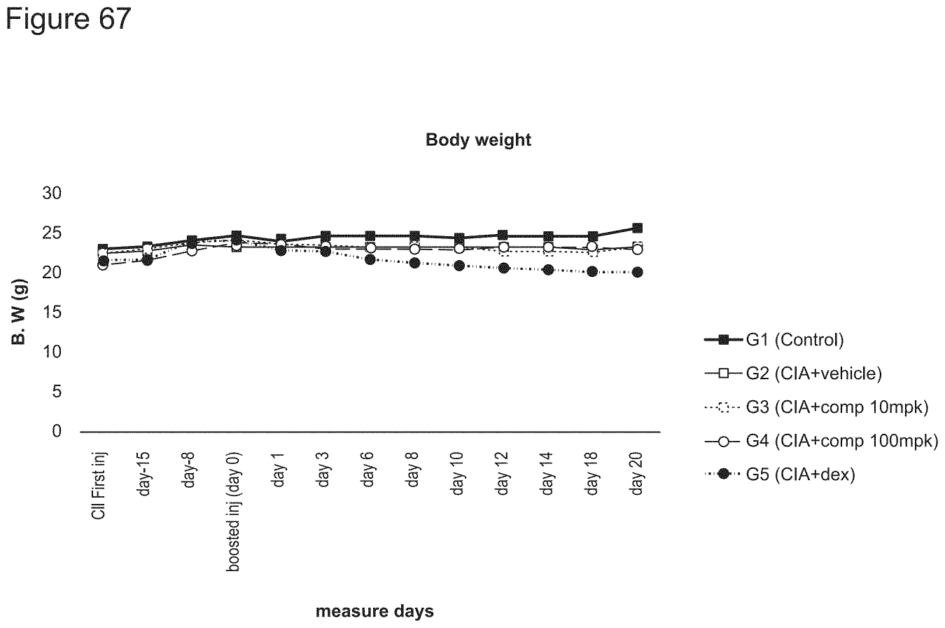

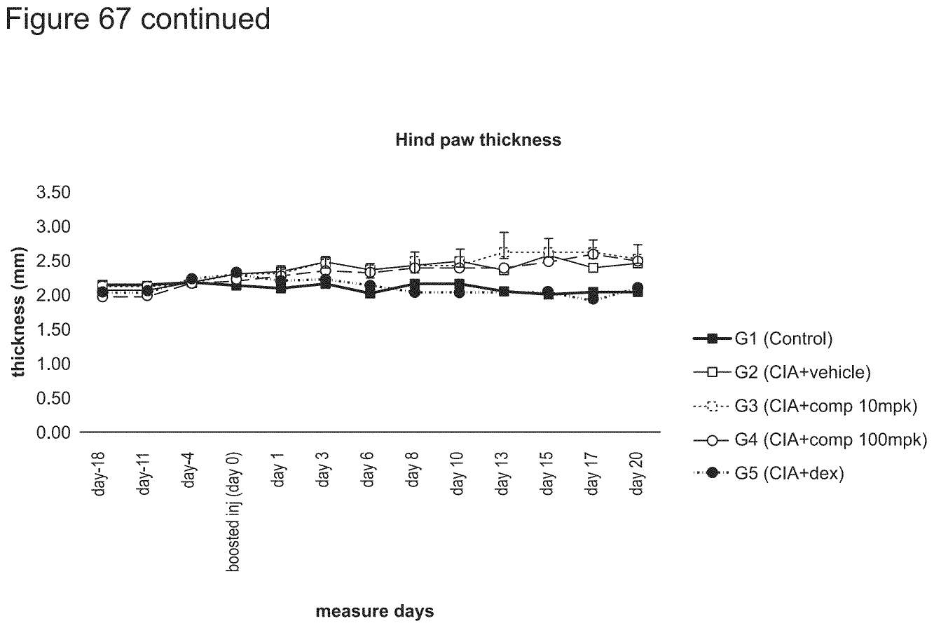

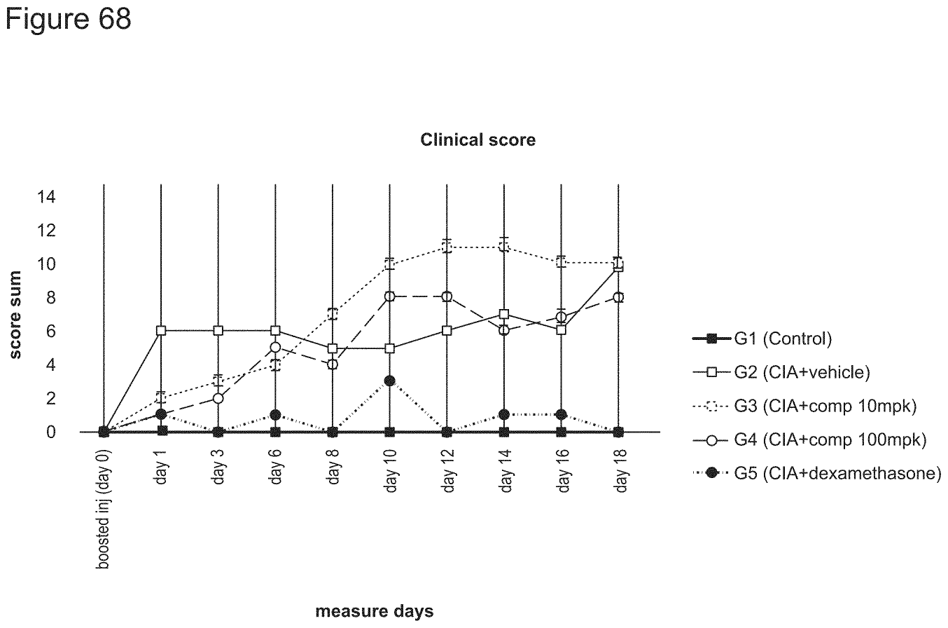

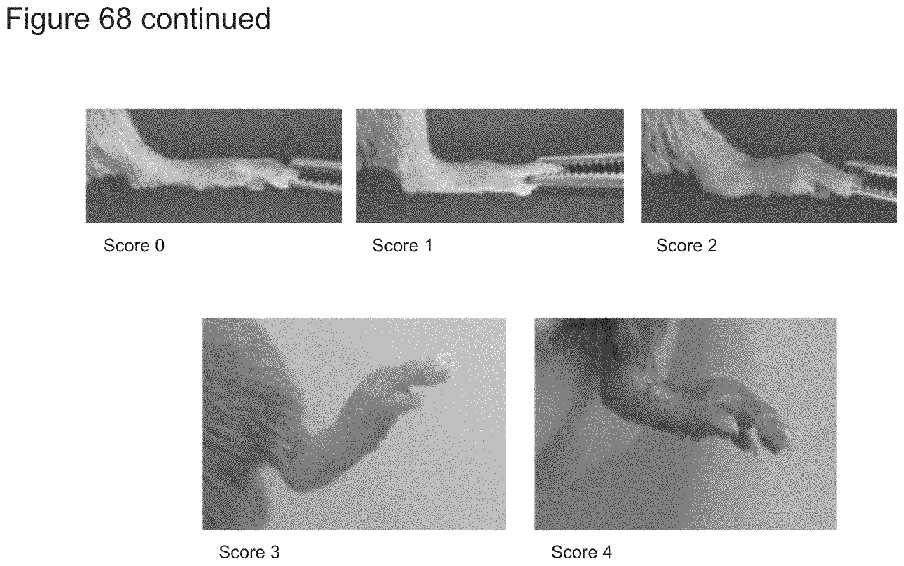

CII injection led to marked induction of arthritis-like symptoms in mice as early as one day after a CII booster injection and continued to escalated symptoms up to 3 weeks.
A reference compound, dexamethasone, strongly reduced inflammation and clinical scores in hind paws although it also lead to a decease in body weight in mice.
YDE-048 reduced the clinical symptoms and scores in the first week (an early phase of inflammation)
Recommend to repeat the study with increased sample size, higher dose (e.g., 300 mg/kg) and/or alternative drug delivery (Alzet pump or Matrigel)
   The data and results are shown in FIGS. 66-68.

### Example 14: Mouse Osteoporosis Model

OVX induced Osteoporosis disease model in C57bL6 mouse

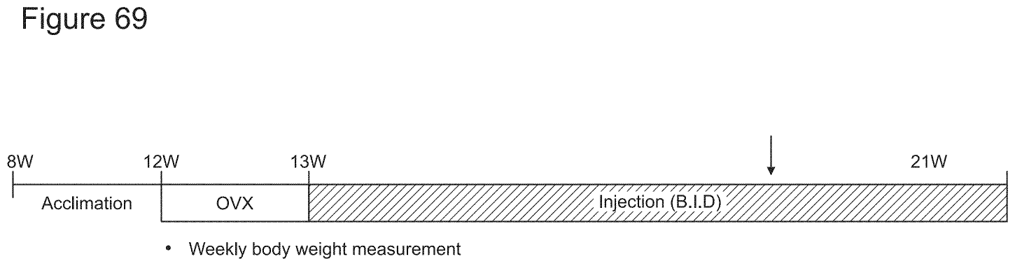

Experiment schematic timeline is shown in FIG. 69.

Study scheme and group information:
12 week old C57bl6/J Female and generate OVX-induced osteoporosis model
Study Length: 9 Weeks (1 week model generation+8 weeks of Test article dosing)
   Acclamation: 1 week
   Dosing: B.I.D/8 weeks
   Body Weight and General observation: 8 weeks
Report preparation: 7 days
Formulation and test article handling and storage (according to client MSDS)
   Body weight measurement (weekly); uterus weight at the end of experiment
   End-point termination:
      Lumbar vertebrae bone strength test
      Femurs fixed in 4% paraformaldehyde for μ-CT (e.g., bone mineral density, bone structure, bone quality and volume)
      Fixed femurs decalcified in 12% EDTA2Na at RT for approximately 3 weeks and embedded in paraffin. Their slices used for TRAP staining, H&E (hematoxylin and eosin) staining, and immunohistochemical staining.
      Body weight
   Total N=28
      4 groups, n=7 in each group, total 28 mice:
      Vehicle, Test article 1, Test article 2
      WTN=7
      Osteoporosis model N=21

End point sampling:
body weight
sampling time after 1 hr compound injection
serum
saline perfusion
uterus->weight, gross
femur (L/R)
   L—in 4% PFA for u-CT (get a sample back)
   R—in 10% NBF for staining
Lumbar vertebrae in PBS for bone test

TABLE 52

| | | | |
|---|---|---|---|
| Group | mpk | N | Route |

| | | | | | |
|---|---|---|---|---|---|
| 1 | WT | | 7 | S.C | B.I.D |
| 2 | OVX + Vehicle | | 7 | S.C | |
| 3 | OVX + YDE-048 | 10 | 7 | S.C | |
| 4 | OVX + YDE-048 | 100 | 7 | S.C | |

YDE-048 is soluble in saline

### Summary

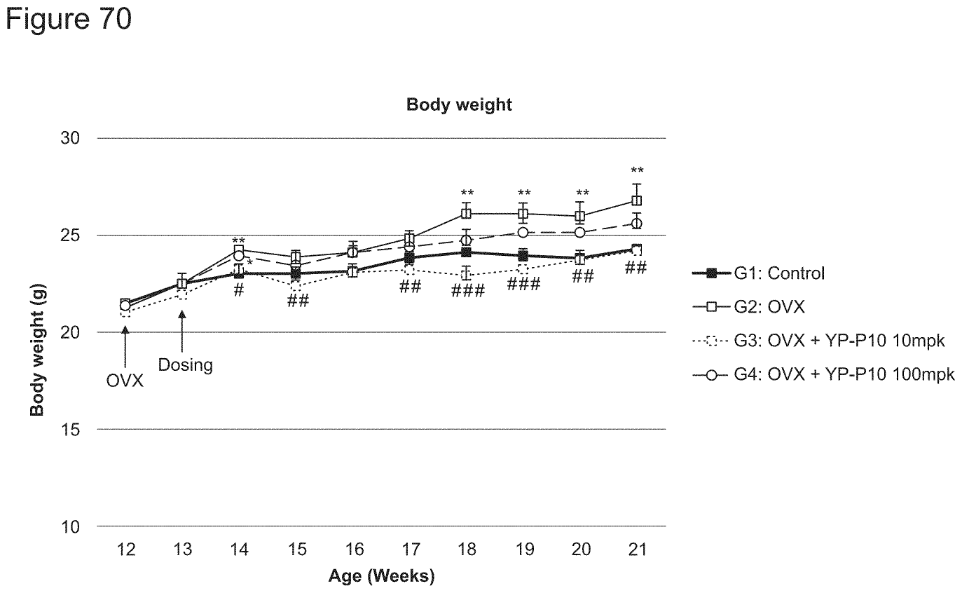

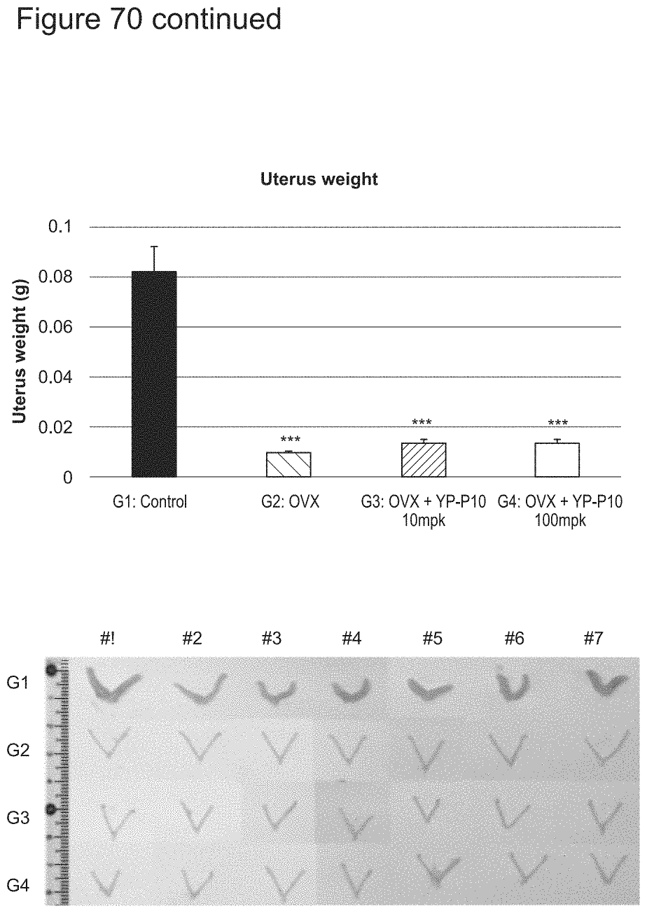

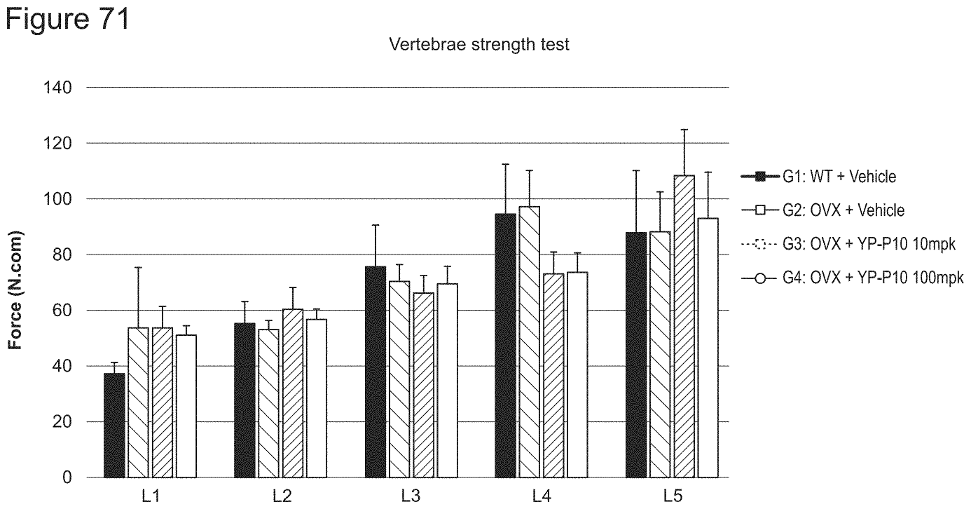

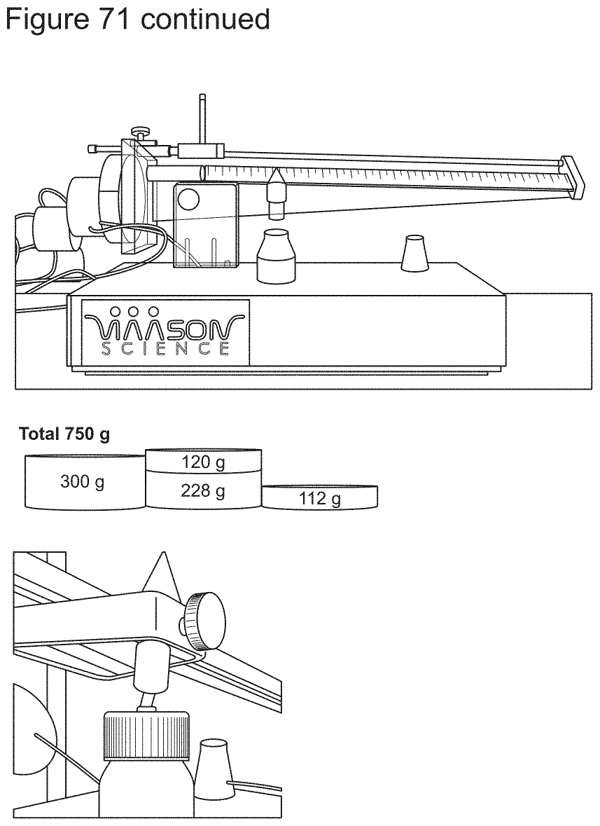

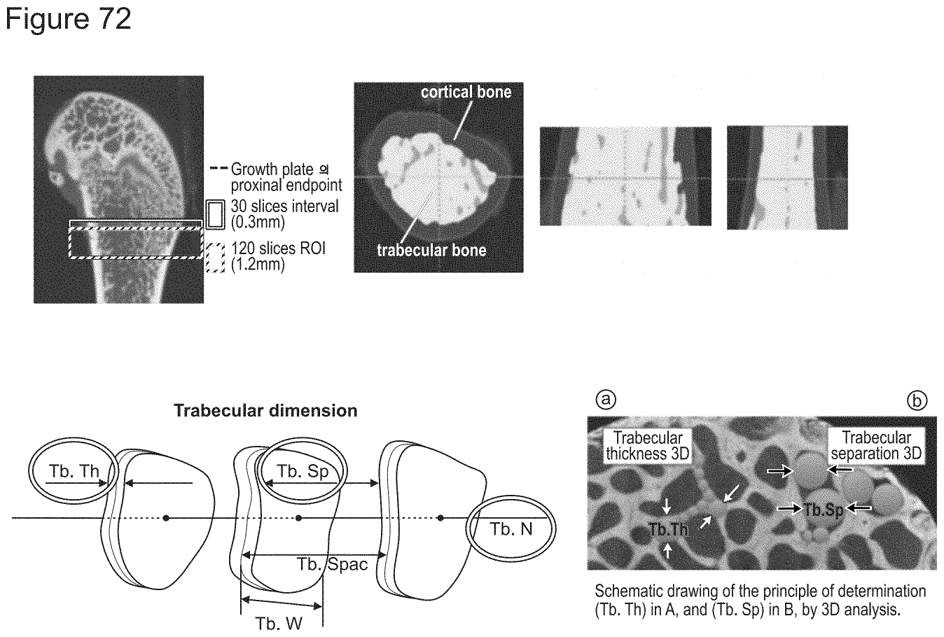

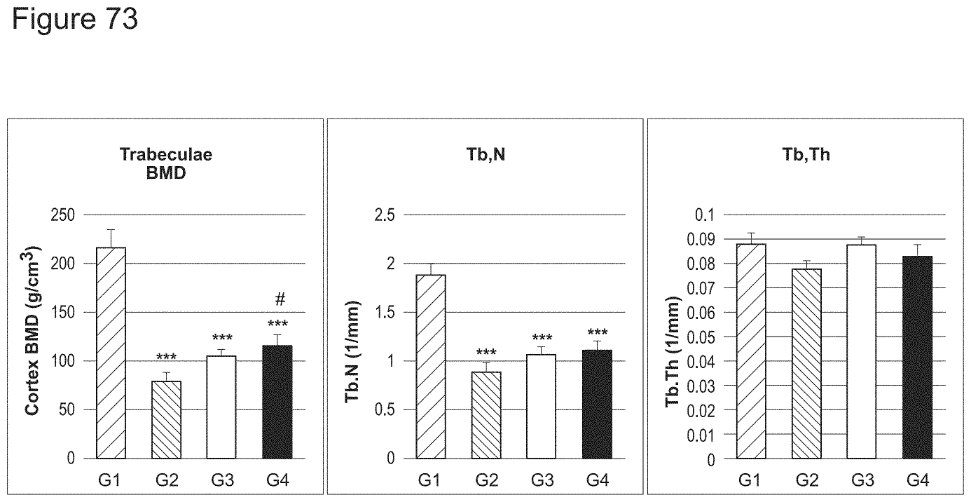

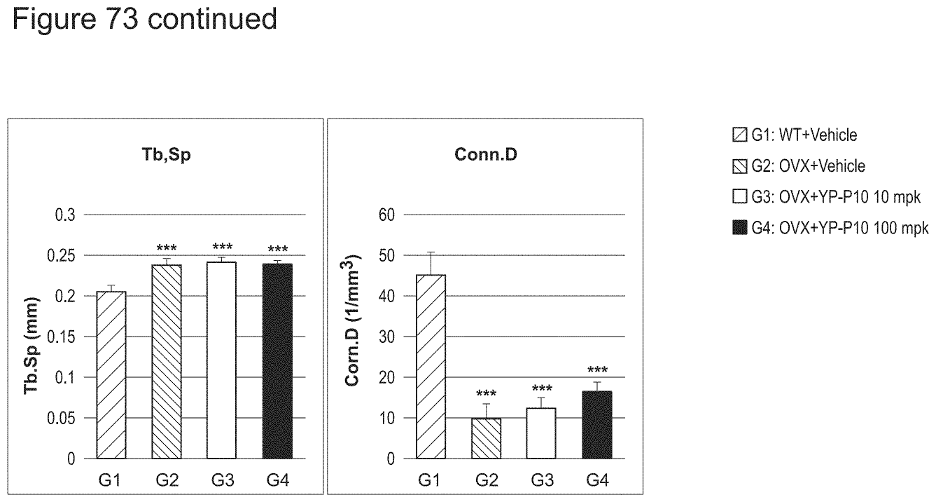

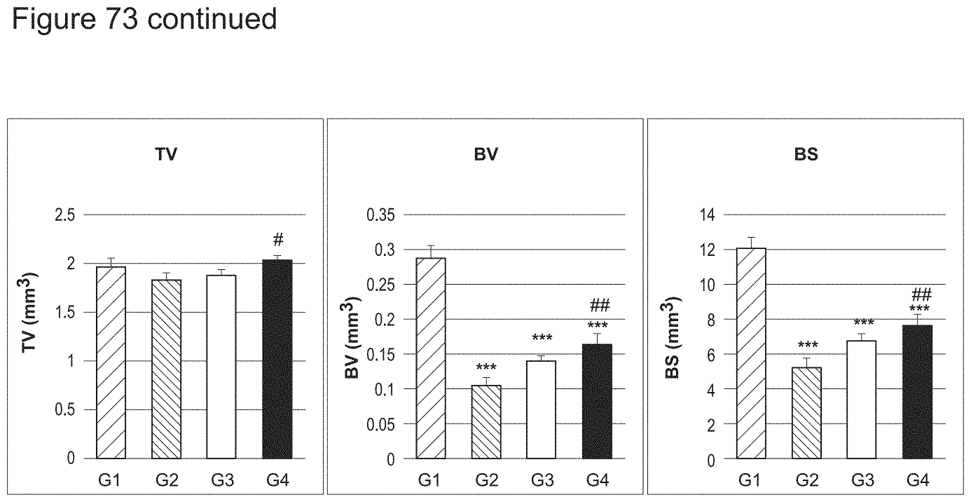

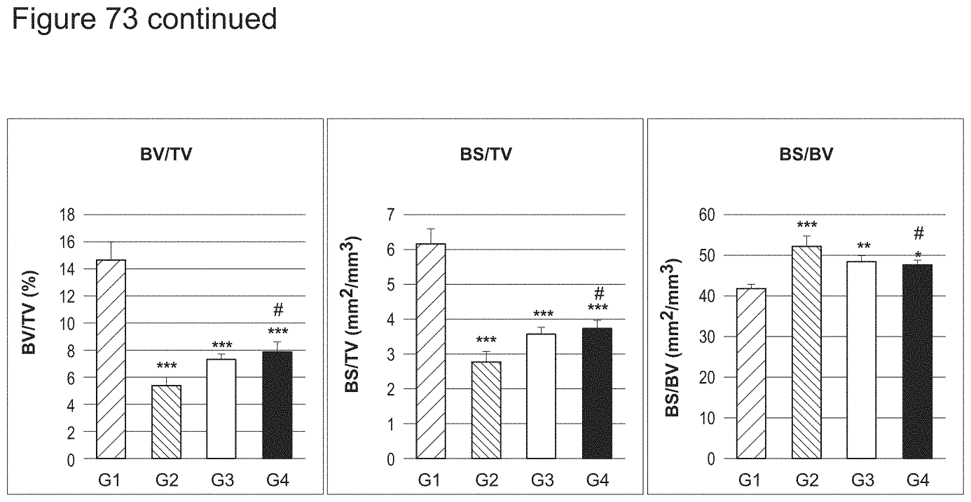

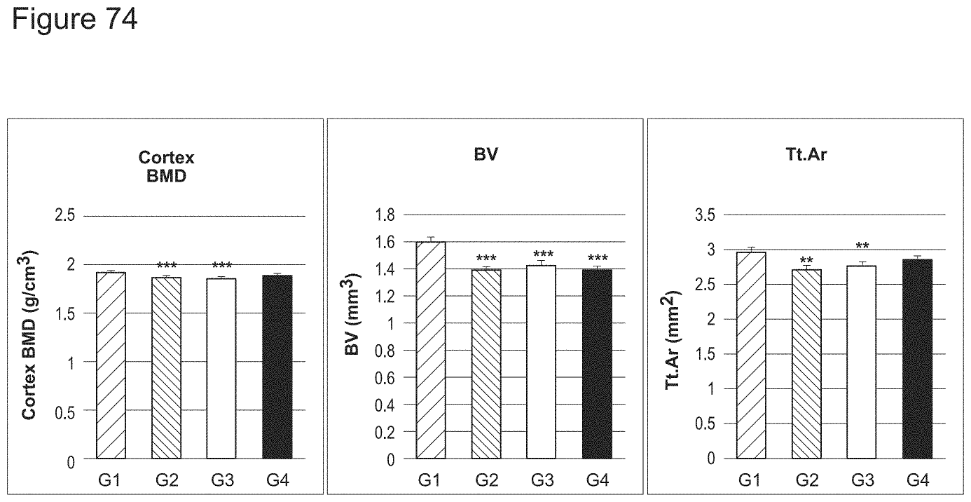

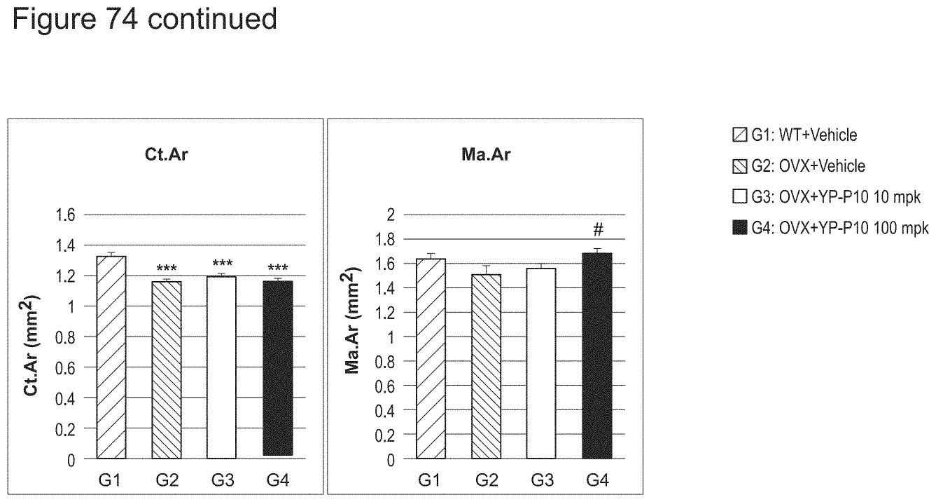

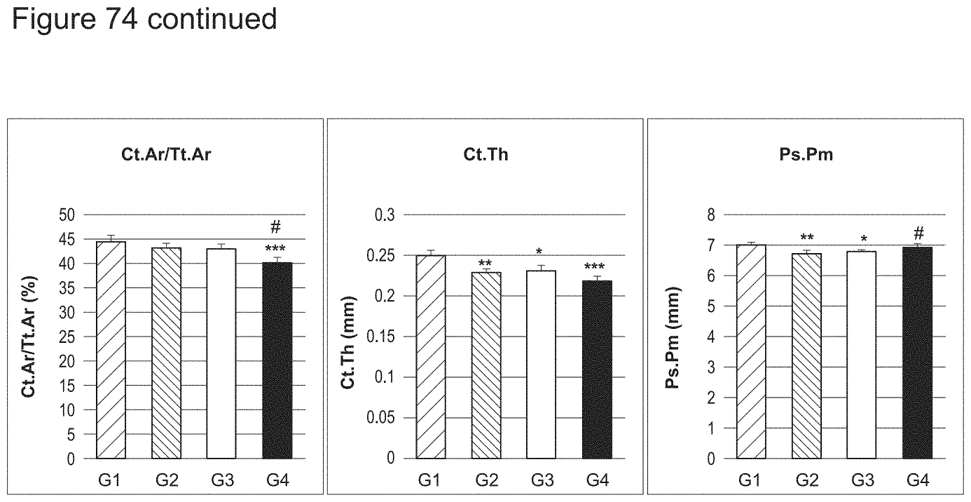

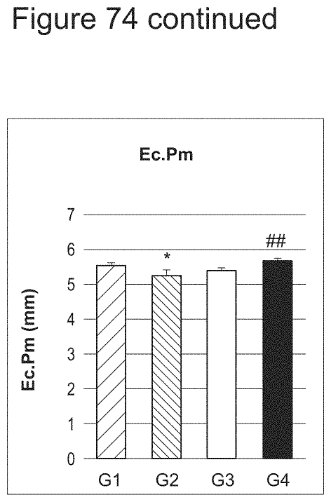

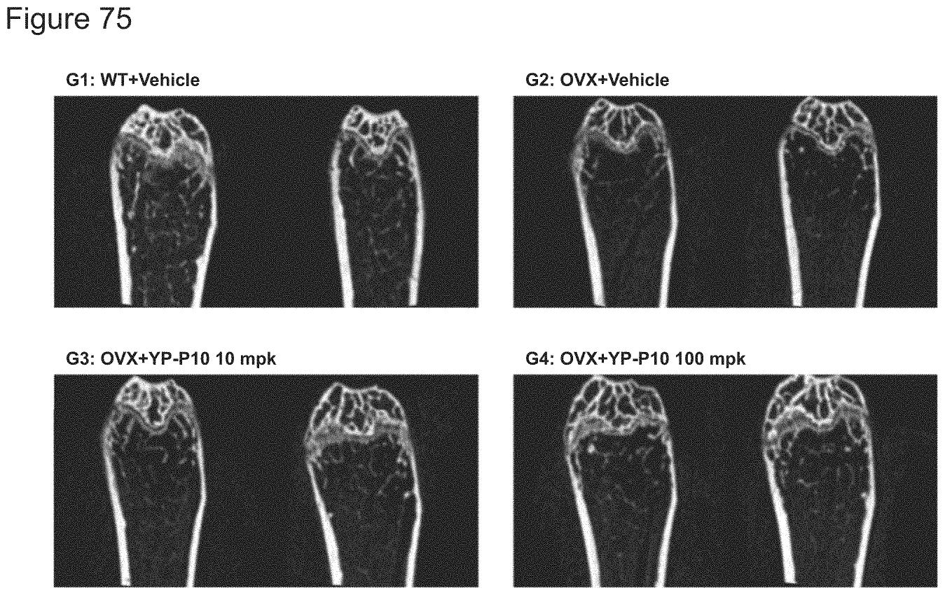

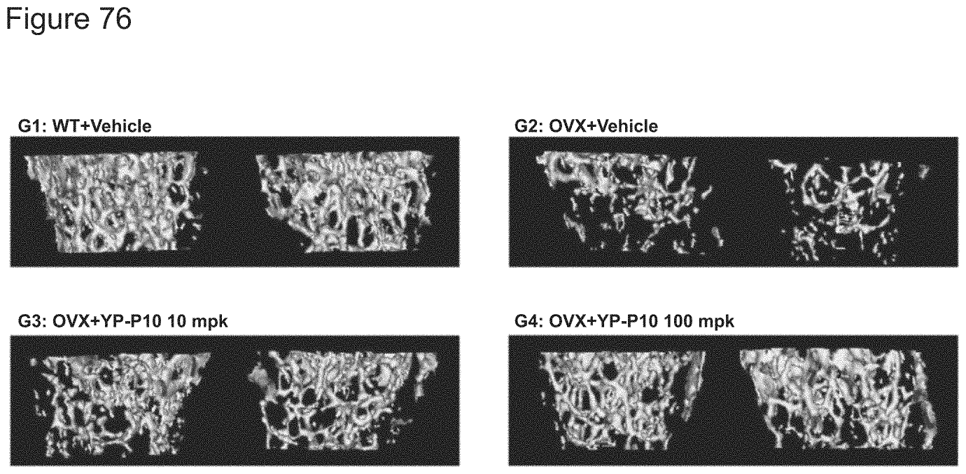

Ovariectomy (OVX) led to a body weight increase, uterus weight loss in female C57BL/6 mice.
Micro-CT scan showed OVX also led to trabecular bone density and connectivity loss in femur with little loss in cortical bone loss in 2 months.
OVX didn't affect lumbar vertebrae bone strength within 2 months.
YDE-048 significantly protected trabecular bone loss (both density and volume) in a dose-dependent manner
Recommend to repeat the study with increased sample size, higher dose (e.g., 300 mg/kg) and/or alternative drug delivery (Alzet pump or Matrigel)
   The data and results are shown in FIGS. 69-76.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of preventing or treating an IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine mediated disease or disorder in a subject, comprising administering to the subject
i) a compound represented by Formula (I), (V), (VI), (VII), (IX) or (X-am) or a pharmaceutically acceptable salt and/or prodrug thereof: 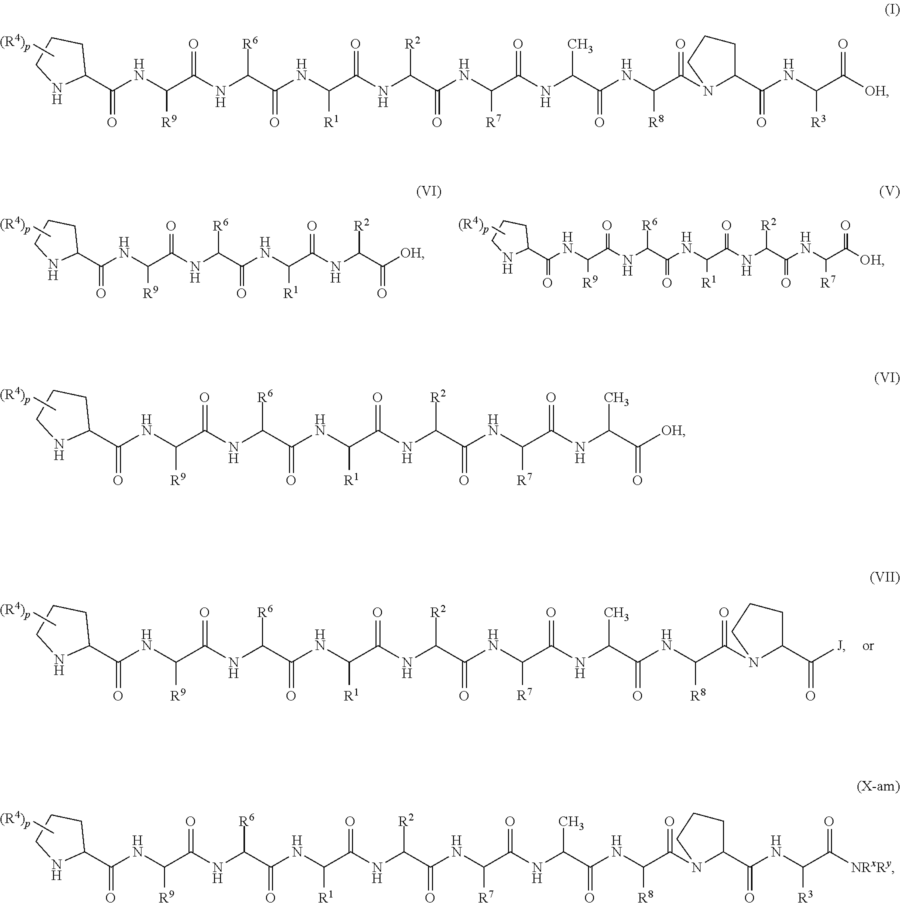
wherein:
R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl;
R⁷, R⁸, and R⁹ are each independently hydrogen or alkyl;
J is OH or —NR^{x}R^{y}; and
R^{x} and R^{y} are each independently selected from H, optionally substituted alkyl, optionally substituted alkoxylalkyl, or R^{x} and R^{y} taken together with the intervening nitrogen atom form a ring,
ii) a compound represented by Formula (8) or (10)
or a pharmaceutically acceptable salt and/or prodrug thereof: 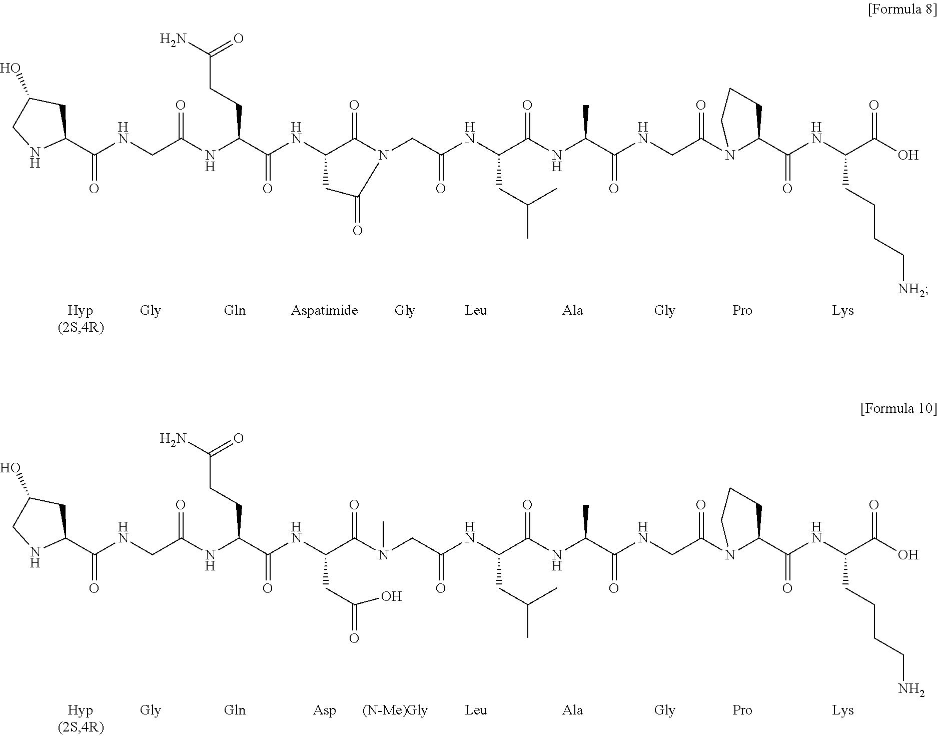 [SMILES: CC(C)C[C@H](NC(=O)CN1C(=O)C[C@H](NC(=O)[C@H](CCC(N)=O)NC(=O)CNC(=O)[C@@H]2C[C@@H](O)CN2)C1=O)C(=O)N[C@@H](C)C(=O)NCC(=O)N1CCC[C@H]1C(=O)N[C@@H](CCCCN)C(=O)O ; CC(C)C[C@H](NC(=O)CN(C)C(=O)[C@H](CC(=O)O)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O]
iii) a peptide having an amino acid sequence represented by HyP-Gly-Gln-Xaa-Gly-Leu-Ala-Gly-Pro-Lys, HyP-Gly-Gln-Asp-Xbb-Leu-Ala-Gly-Pro-Lys, HyP-Gly-Gln-Leu-Gly-Leu-Ala-Gly-Pro-Xcc or Xdd-Gly-Gln-Leu-Gly-Leu-Ala-Gly-Pro-Lys, or a pharmaceutically acceptable salt and/or stereoisomer thereof,
wherein:
Xaa is selected from Glu, Asn, Gln, His, Lys, Ser, Thr, Ala, Val, Ile, Leu, Phe, Tyr, Trp, homo-Ser, Asp(Me), and Asn(Me);
Xbb is selected from Val, Ile, Leu, Ala, Phe, Tyr, Trp, Ser, Thr, and (N-Me)Gly;
Xcc is selected from Tyr, Leu, Glu, Gln, Ala, and Nle(6-OH); and
Xdd is selected from: 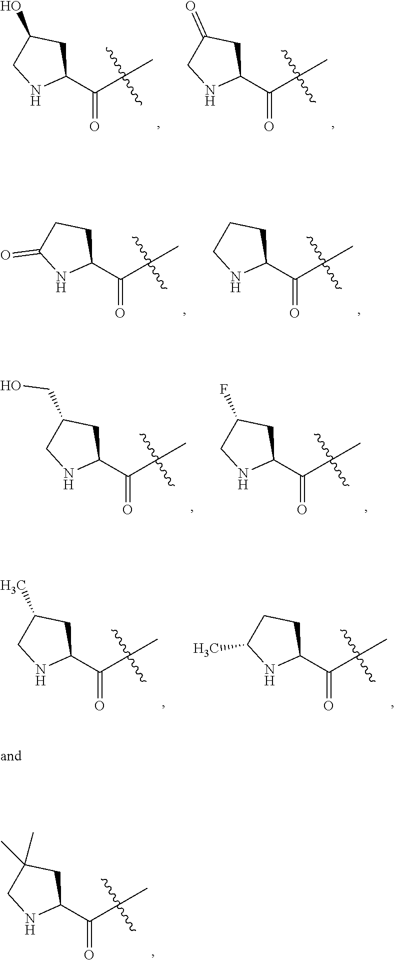
or
iv) a peptide having any one amino acid sequence selected from:
Ala-Gly-Gln-Leu-Gly-Leu-Ala-Gly-Pro-Lys;
Hyp-Gly-Gln-Leu-Gly-Leu-Ala-Gly-Ala-Lys;
HyP-Gly-Gln-Leu-Gly-Leu-Ala;
HyP-Gly-Gln-Glu-Gly-Leu-Gly;
HyP-Gly-Gln-Leu-Gly-Leu;
_{D}-HyP(2R, 4S)-Gly-_{D}-Gln-_{D}-Leu-Gly-_{D}-Leu;
HyP-Gly-Gln-Leu-Gly, HyP-Gly-Gln-_{D}-Leu-Gly; and
_{D}-HyP(2R, 4S)-Gly-Gln-Leu-Gly.

2. The method of claim 1, wherein the subject has elevated levels of IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine.

3. The method of claim 1 or 2, wherein the IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine mediated disease is an autoimmune disease, an inflammatory disease, or a cancer.

4. The method of any one of claims 1-3, wherein the disease or disorder is Acute posterior multifocal placoid pigment epitheliopathy (APMPPE), Agammaglobulinemia, Alopecia Areata, Amyloidosis, Amyotrophic lateral sclerosis (ALS), Aniridia, Ankylosing spondylitis, Anti-GBM/Anti-TBM nephritis, Antiphospholipid syndrome, Autoimmune hepatitis, Autoimmune inner ear disease, Autoimmune Lymphoproliferative Syndrome, Atopic dermatitis, Asthma, Behçet's Disease, Best Disease, Birdshot Chorioretinopathy, Blepharitis, Bronchiolitis, Cancer (Chondrosarcoma, Cervical, Breast, Lung), Capillary Leak Syndrome, Castleman disease, Celiac disease, Chagas disease, Chalazia and Stye, Chandler's syndrome, Cholesteatoma of Middle Ear, Choroideremia, Chronic recurrent multifocal osteomyelitis, Cogan's syndrome, Collagen Induced Arthritis (CIA), Cold agglutinin disease, Cone Rod Dystrophies, Conjunctivitis, Corneal Wound Healing, CREST syndrome, Crohn's disease, Dermatomyositis, Devic's disease (neuromyelitis optica), Discoid lupus, Dry Eye Disease (DED), Dry macular degeneration (Dry AMD), Endometriosis, Eosinophilic esophagitis, Eosinophilic fasciitis, Essential Iris Atrophy, Evan's syndrome, Farmer's Lung, Fibromyalgia, Giant cell arteritis, Giant cell myocarditis, Giant Papillary Conjunctivitis, Glomerulonephritis, Goodpasture's syndrome, Graft-Versus-Host Disease, Granulomatosis with polyangiitis, Graves' disease, Guillain-Barre syndrome, Gyrate Atrophy, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Iritis, Irritant Dermatitis, Juvenile arthritis, Juvenile/Type 1 Diabetes, Juvenile macular degeneration, Juvenile myositis, Juvenile X-linked Retinoschisis, Kawasaki syndrome, Keratitis, Keratoconjunctivitis sicca (Dry Eye), Late-Onset Retinal Degeneration (L-ORD), Lichen planus, Lichen sclerosus, Lupus (SLE), Macular Edema, Meniere's disease, Multiple sclerosis, Myasthenia gravis, Microscopic polyangiitis, Neuropathic Corneal Pain, Neurotropic Keratitis, Ocular Allergy, Ocular Inflammation (uveitis), Ocular Pain, Ocular Neurodegeneration, Optic Nerve Atrophy, Optic neuritis, Oral Submucous Fibrosis, Osteroarthritis (OA), Osteoporosis, Parkinson's disease, Pars Planitis, Pemphigus, Photokeratitis, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Scleritis, Sjogren's syndrome, Stargardt Disease, Stickler Syndrome, Temporal arteritis/Giant cell arteritis, Thyroid Eye Disease, Trachoma, Transverse myelitis, Trichiasis, Ulcerative colitis, Usher Syndrome, Uveitis, Vasculitis, Vitiligo, Viral myocarditis, Wegener's granulomatosis (Granulomatosis with Polyangiitis (GPA)), Wet macular degeneration, or Wound Healing.

5. A method of reducing production of IL-1α, IL-1β, IL-2, IL-6, IL-8, IL-10, TNF-α, MMP3, CCL-2, CCL-3, CCL-4, Fas, TIMP-1, or a Th1, Th2, and/or Th17 derived cytokine or proinflammatory chemokine in cells of a subject, comprising administering to a subject
i) a compound represented by Formula (I), (V), (VI), (VII), (IX) or (X-am) or a pharmaceutically acceptable salt and/or prodrug thereof: 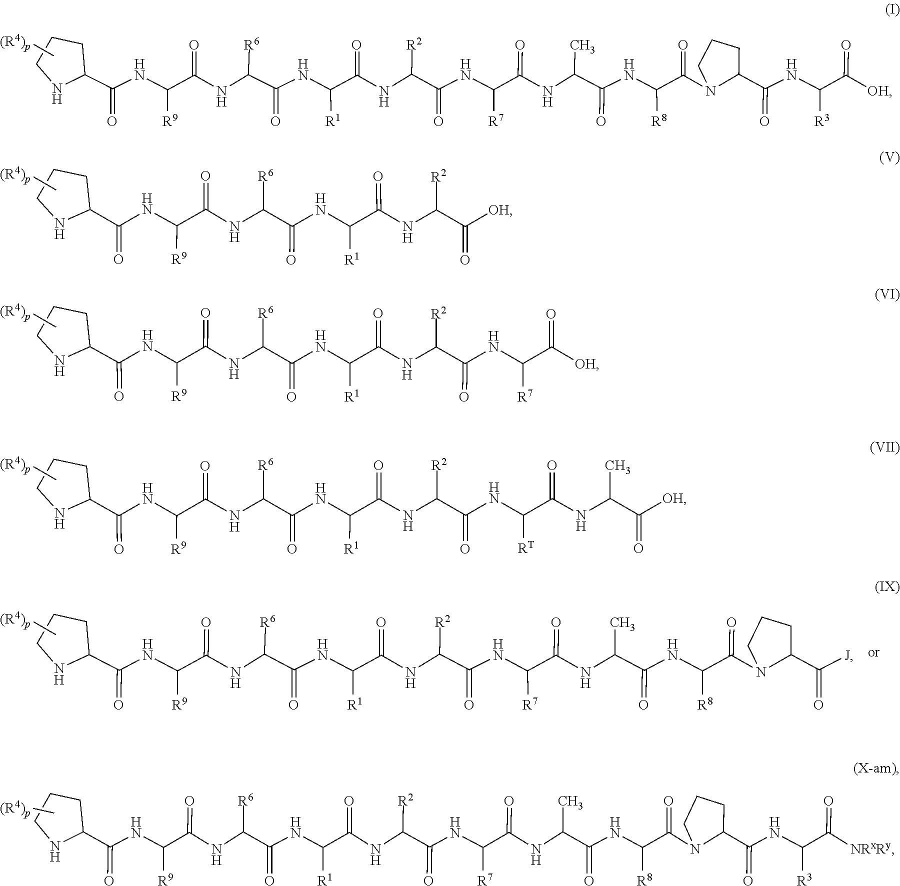
wherein:
R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl;
R⁷, R⁸, and R⁹ are each independently hydrogen or alkyl;
J is OH or —NR^{x}R^{y}; and
R^{x} and R^{y} are each independently selected from H, optionally substituted alkyl, optionally substituted alkoxylalkyl, or R^{x} and R^{y} taken together with the intervening nitrogen atom form a ring,
ii) a compound represented by Formula (8) or (10)
or a pharmaceutically acceptable salt and/or prodrug thereof: 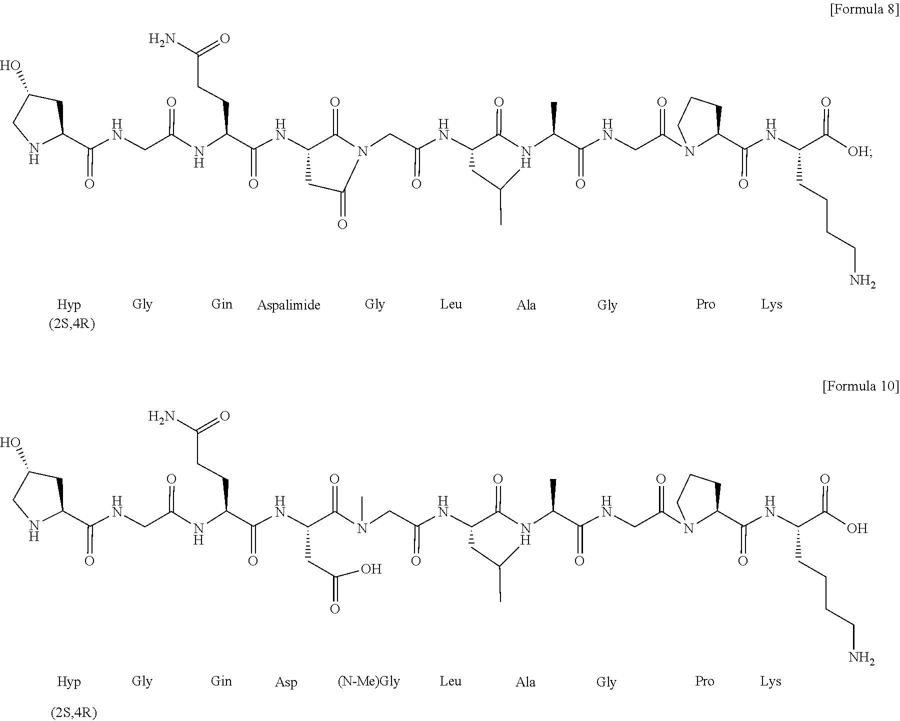
iii) a peptide having an amino acid sequence represented by HyP-Gly-Gln-Xaa-Gly-Leu-Ala-Gly-Pro-Lys, HyP-Gly-Gln-Asp-Xbb-Leu-Ala-Gly-Pro-Lys, HyP-Gly-Gln-Leu-Gly-Leu-Ala-Gly-Pro-Xcc or Xdd-Gly-Gln-Leu-Gly-Leu-Ala-Gly-Pro-Lys, or a pharmaceutically acceptable salt and/or stereoisomer thereof,
wherein:
Xaa is selected from Glu, Asn, Gln, His, Lys, Ser, Thr, Ala, Val, Ile, Leu, Phe, Tyr, Trp, homo-Ser, Asp(Me), and Asn(Me);
Xbb is selected from Val, Ile, Leu, Ala, Phe, Tyr, Trp, Ser, Thr, and (N-Me)Gly;
Xcc is selected from Tyr, Leu, Glu, Gln, Ala, and Nle(6-OH); and
Xdd is selected from: 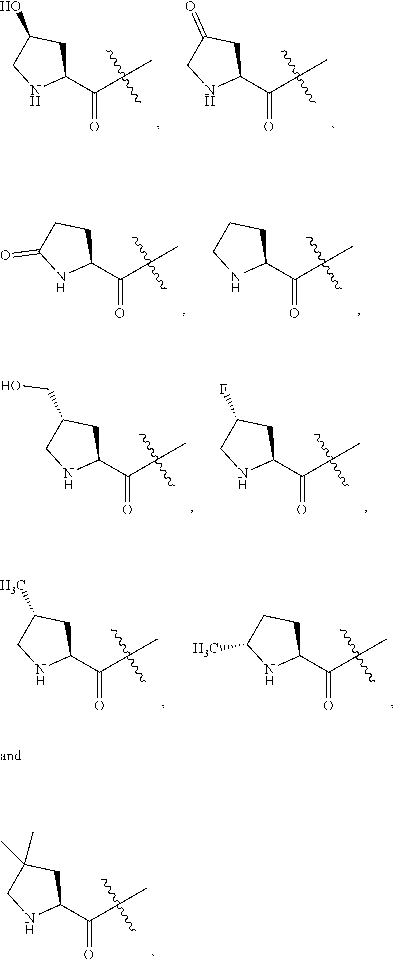
or
iv) a peptide having any one amino acid sequence selected from:
Ala-Gly-Gln-Leu-Gly-Leu-Ala-Gly-Pro-Lys;
Hyp-Gly-Gln-Leu-Gly-Leu-Ala-Gly-Ala-Lys;
HyP-Gly-Gln-Leu-Gly-Leu-Ala;
HyP-Gly-Gln-Glu-Gly-Leu-Gly;
HyP-Gly-Gln-Leu-Gly-Leu;
_{D}-HyP(2R, 4S)-Gly-_{D}-Gln-_{D}-Leu-Gly-_{D}-Leu;
HyP-Gly-Gln-Leu-Gly, HyP-Gly-Gln-_{D}-Leu-Gly; and
_{D}-HyP(2R, 4S)-Gly-Gln-Leu-Gly.

6. The method of claim 5, wherein administering the compound reduces the cytokine and/or chemokine levels by at least 30% compared to the untreated control.

7. The method of claim 5, wherein administering the compound reduces the cytokine and/or chemokine levels by at least 50% compared to the untreated control.

8. The method of claim 5, wherein administering the compound reduces the cytokine and/or chemokine levels by at least 70% compared to the untreated control.

9. A method of reducing NF-κB transcription activity in cells of a subject, comprising administering to a subject
i) a compound represented by Formula (I), (V), (VI), (VII), (IX) or (X-am) or a pharmaceutically acceptable salt and/or prodrug thereof: 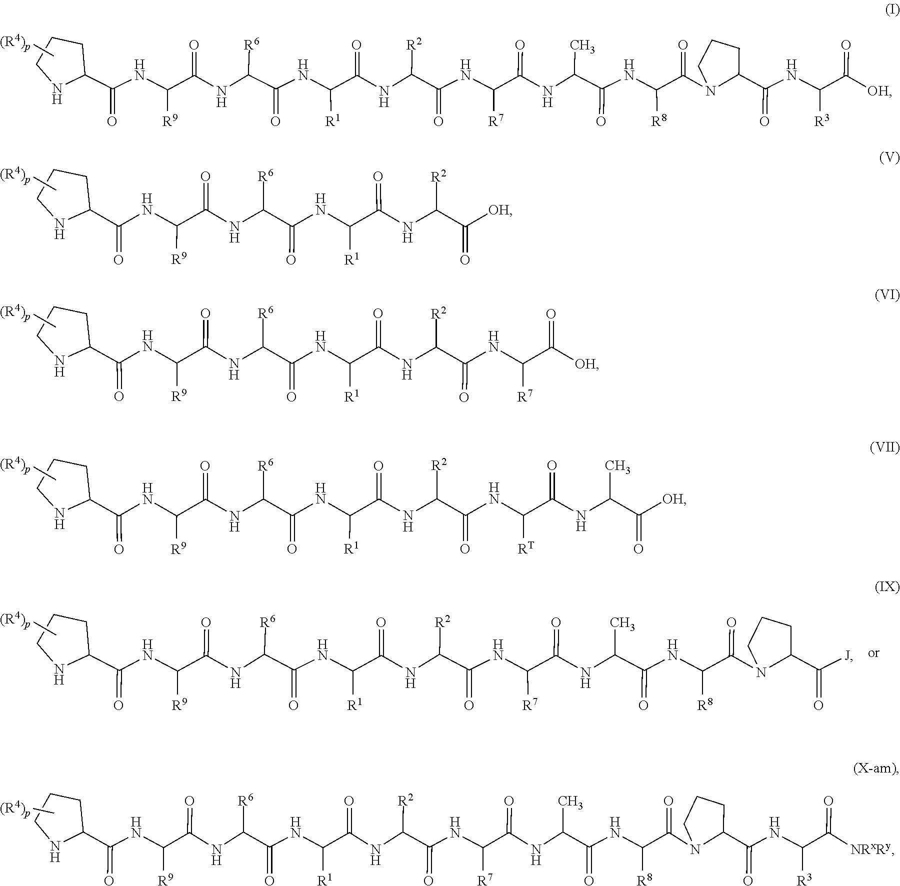
wherein:
R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl;
R⁷, R⁸, and R⁹ are each independently hydrogen or alkyl;
J is OH or —NR^{x}R^{y}; and
R^{x} and R^{y} are each independently selected from H, optionally substituted alkyl, optionally substituted alkoxylalkyl, or R^{x} and R^{y} taken together with the intervening nitrogen atom form a ring,
ii) a compound represented by Formula 8 or 10
or a pharmaceutically acceptable salt and/or prodrug thereof: 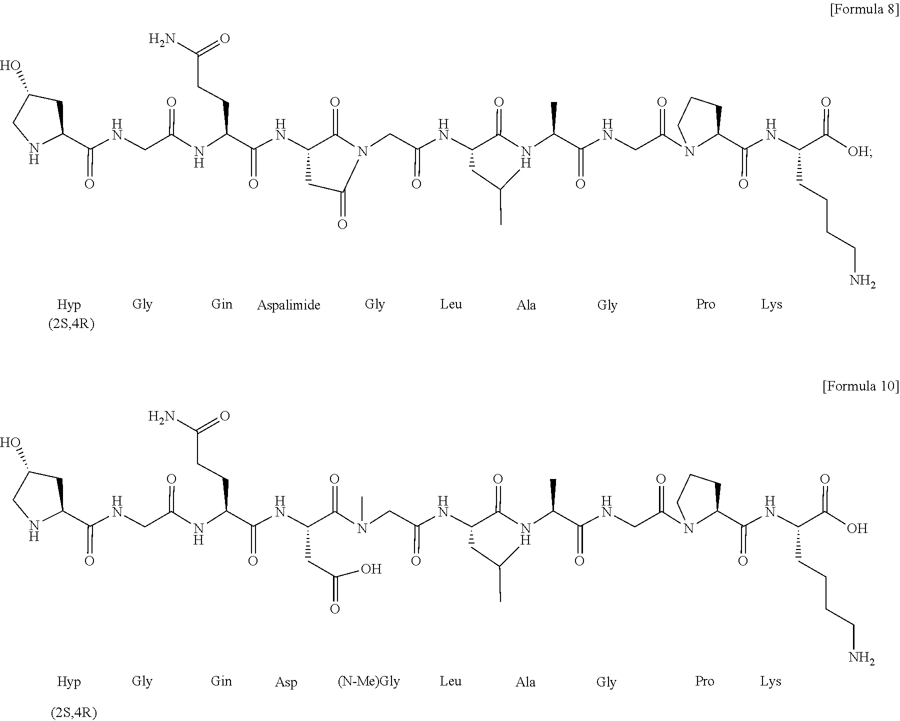
iii) a peptide having an amino acid sequence represented by HyP-Gly-Gln-Xaa-Gly-Leu-Ala-Gly-Pro-Lys, HyP-Gly-Gln-Asp-Xbb-Leu-Ala-Gly-Pro-Lys, HyP-Gly-Gln-Leu-Gly-Leu-Ala-Gly-Pro-Xcc or Xdd-Gly-Gln-Leu-Gly-Leu-Ala-Gly-Pro-Lys, or a pharmaceutically acceptable salt and/or stereoisomer thereof,
wherein:
Xaa is selected from Glu, Asn, Gln, His, Lys, Ser, Thr, Ala, Val, Ile, Leu, Phe, Tyr, Trp, homo-Ser, Asp(Me), and Asn(Me);
Xbb is selected from Val, Ile, Leu, Ala, Phe, Tyr, Trp, Ser, Thr, and (N-Me)Gly;
Xcc is selected from Tyr, Leu, Glu, Gln, Ala, and Nle(6-OH); and
Xdd is selected from: 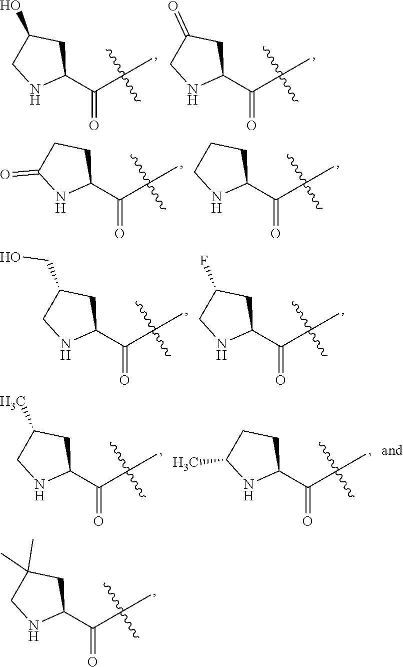
or
iv) a peptide having any one amino acid sequence selected from:
Ala-Gly-Gln-Leu-Gly-Leu-Ala-Gly-Pro-Lys;
Hyp-Gly-Gln-Leu-Gly-Leu-Ala-Gly-Ala-Lys;
HyP-Gly-Gln-Leu-Gly-Leu-Ala;
HyP-Gly-Gln-Glu-Gly-Leu-Gly;
HyP-Gly-Gln-Leu-Gly-Leu;
_{D}-HyP(2R, 4S)-Gly-_{D}-Gln-_{D}-Leu-Gly-_{D}-Leu;
HyP-Gly-Gln-Leu-Gly, HyP-Gly-Gln-_{D}-Leu-Gly; and
_{D}-HyP(2R, 4S)-Gly-Gln-Leu-Gly.

10. The method of any one of claims 1-9, wherein the subject is a mammal.

11. The method of claim 10, wherein the mammal is a mouse or a human.

12. The method of claim 11, wherein the mammal is a human.

13. The method of any one of claims 1-12, wherein the compound is represented by Formula (I): 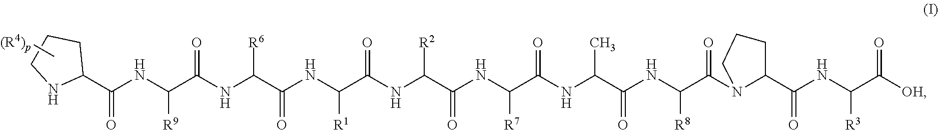
or a pharmaceutically acceptable salt and/or prodrug thereof,
wherein:
R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl; and
R⁷, R⁸, and R⁹ are each independently hydrogen or alkyl.

14. The method of claim 13, wherein:
R¹, R², and R³ are each independently H or substituted or unsubstituted alkyl, arylalkyl, or heteroarylalkyl;
R⁴, independently for each occurrence, is selected from substituted or unsubstituted alkyl, oxo, hydroxyl, —OR^{b}, hydroxyalkyl, —CH₂OR^{b}, and halo;
R^{b} is substituted or unsubstituted alkyl, aryl, arylalkyl, or heterocycloalkyl;
R⁶ is hydrogen or substituted or unsubstituted alkyl; and
R⁷, R⁸, and R⁹ are each independently hydrogen or alkyl.

15. The method of claim 13 or 14, wherein, where indicated, alkyl, alkoxy, haloalkyl, alkenyl, alkynyl, aryl, arylalkyl, arylalkenyl, arylalkynyl, heteroalkyl, cycloalkyl, heterocycloalkyl, or heteroaryl is unsubstituted or is substituted with one or more substituents selected from halo, haloalkyl, oxo, —CN, —NO₂, ═N—OH, —N₃, —R^{a}, —OR^{a}, —SR^{a}, —N(R^{a})₂, —N(R^{a})₃⁺, ═NR^{a}, —NHC(═O)R^{c}, —C(═O)R^{c}, —C(═O)N(R^{a})₂, —S(═O)₂R^{c}, —OS(═O)₂OR^{a}, —S(═O)₂OR^{a}, —S(═O)₂N(R^{a})₂, —S(═O)R^{c}, —OP(═O)(OR^{a})₂, -(alkylene)-C(═O)R^{c}, —C(═S)R^{c}, —C(═O)OR^{a}, -(alkylene)-C(═O)OR^{a}, —C(═S)OR^{a}, —C(═O)SR^{a}, —C(═S)SR^{a}, -(alkylene)-C(═O)N(R^{a})₂, —C(═S)N(R^{a})₂, and —C(—NR^{a})N(R^{a})₂;
R^{a}, independently for each occurrence, is hydrogen, or substituted or unsubstituted alkyl, aryl, arylalkyl, heteroaryl, heteroarylalkyl, cycloalkyl, (cycloalkyl)alkyl, heterocycloalkyl, or (heterocycloalkyl)alkyl; and
R^{c}, independently for each occurrence, is substituted or unsubstituted alkyl, aryl, arylalkyl, heterocyclyl, heterocyclylalkyl, cycloalkyl, or (cycloalkyl)alkyl.

16. The method of any one of claims 13-15, wherein, where indicated, alkyl, alkoxy, haloalkyl, alkenyl, alkynyl, aryl, arylalkyl, arylalkenyl, arylalkynyl, heteroalkyl, cycloalkyl, heterocycloalkyl, or heteroaryl is unsubstituted or is substituted with one or more substituents selected from halo, haloalkyl, oxo, —R^{a}, —OR^{a}, —N(R^{a})₂, —N(R^{a})₃⁺, —NHC(═O)R^{c}, —C(═O)R^{c}, —C(═O)N(R^{a})₂, —C(═O)OR^{a}, -(alkylene)-C(═O)OR^{a}, and -(alkylene)-C(═O)N(R^{a})₂; and
R^{a}, independently for each occurrence, is hydrogen, or substituted or unsubstituted alkyl, aryl, arylalkyl, heteroaryl, heteroarylalkyl, cycloalkyl, (cycloalkyl)alkyl, heterocycloalkyl, or (heterocycloalkyl)alkyl; and
R^{c}, independently for each occurrence, is substituted or unsubstituted alkyl, aryl, arylalkyl, heterocyclyl, heterocyclylalkyl, cycloalkyl, or (cycloalkyl)alkyl.

17. The method of claim 15 or 16, wherein Rₐ, independently for each occurrence, is hydrogen, alkyl, aryl, arylalkyl, heteroaryl, heteroarylalkyl, heterocycloalkyl, or (heterocycloalkyl)alkyl; and
R^{c}, independently for each occurrence, is alkyl, aryl, arylalkyl, heterocyclyl, or heterocyclylalkyl.

18. The method of any one of claims 13-17, wherein the compound has the structure of formula (I-10L):

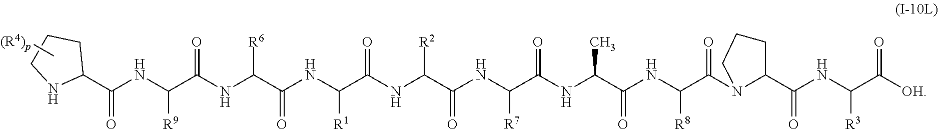

19. The method of any one of claims 13-17, wherein the compound has the structure of formula (I-10D):

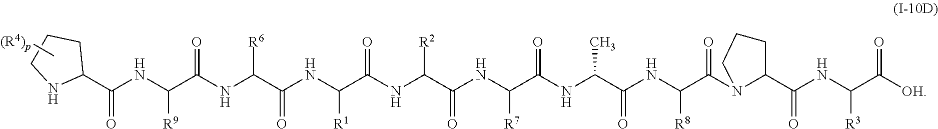

20. The method of any one of claims 13-19, wherein R¹ is substituted or unsubstituted alkyl, arylalkyl, or heteroarylalkyl.

21. The method of any one of claims 13-20 wherein R¹ is selected from substituted or unsubstituted alkyl, 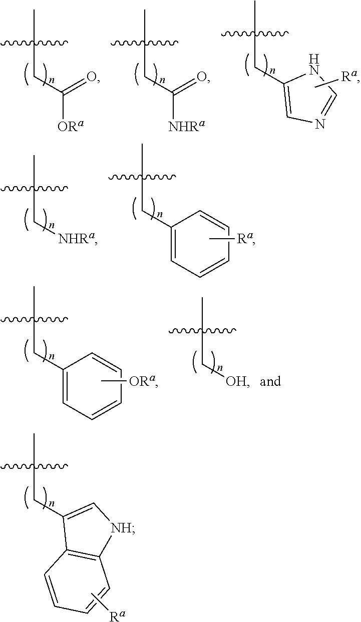
R^{a} is hydrogen or alkyl; and
n is an integer from 1 to 10, preferably 1-5, more preferably 1-3.

22. The method of any one of claims 13-21, wherein R¹ is selected from

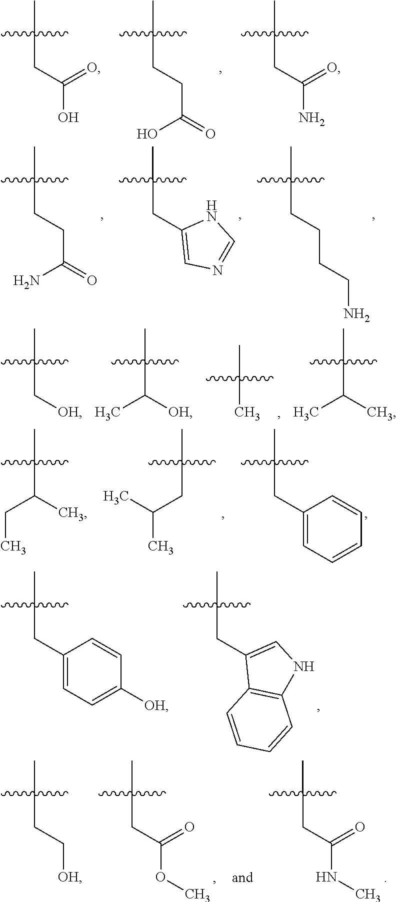

23. The method of any one of claims 13-22, wherein R¹ is

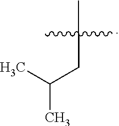 [SMILES: *CC(C)C]

24. The method of any one of claims 13-22, wherein R¹ is OH

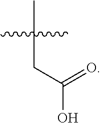 [SMILES: *CC(=O)O]

25. The method of any one of claims 13-24, wherein the compound has the structure of formula (I-1L)

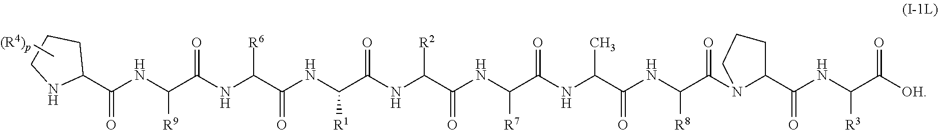

26. The method of any one of claims 13-24, wherein the compound has the structure of formula (I-1D)

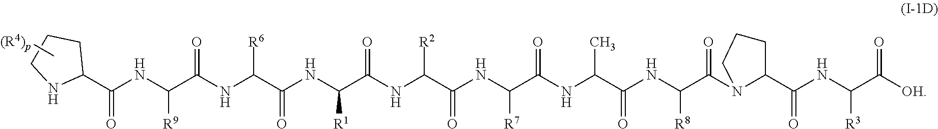

27. The method of any one of claims 13-26, wherein R² is H or substituted or unsubstituted alkyl, arylalkyl, or heteroarylalkyl.

28. The method of any one of claims 13-27, wherein R² is selected from hydrogen, substituted or unsubstituted alkyl, 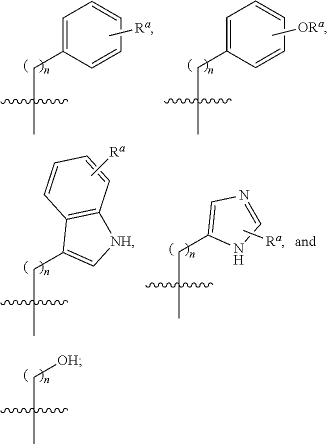
R^{a} is hydrogen or alkyl; and
n is an integer from 1 to 10, preferably 1-5, more preferably 1-3.

29. The method of any one of claims 13-28, wherein R² is selected from

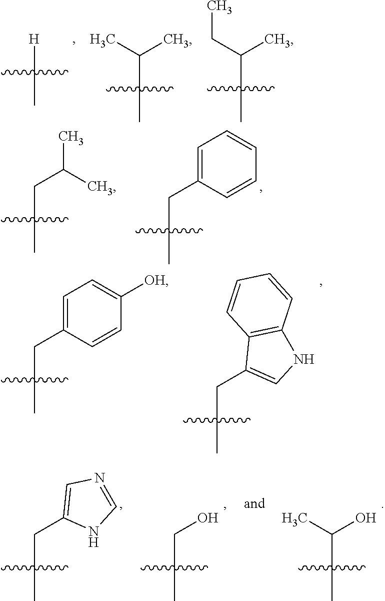

30. The method of any one of claims 13-29, wherein R² is hydrogen.

31. The method of any one of claims 13-30, wherein the compound has the structure of formula (I-2L):

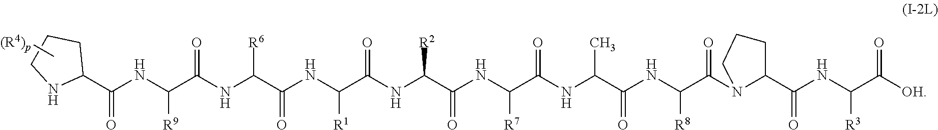

32. The method of any one of claims 13-30, wherein the compound has the structure of formula (I-2D):

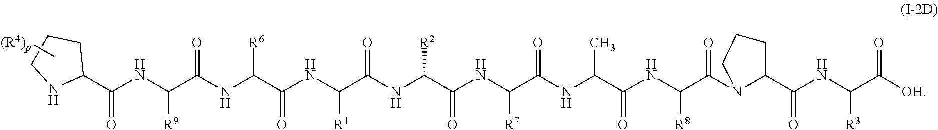

33. The method of any one of claims 13-32, wherein R³ is substituted or unsubstituted alkyl or arylalkyl.

34. The method of any one of claims 13-33, wherein R³ is selected from substituted or unsubstituted alkyl, 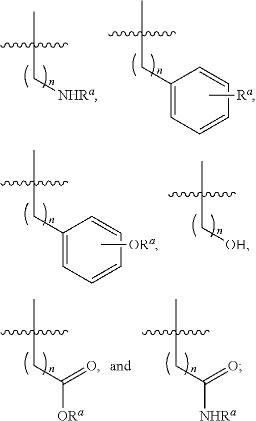
R^{a} is hydrogen or alkyl; and
n is an integer from 1 to 10, preferably 1-5, more preferably 1-3.

35. The method of any one of claims 13-34, wherein R³ is selected from

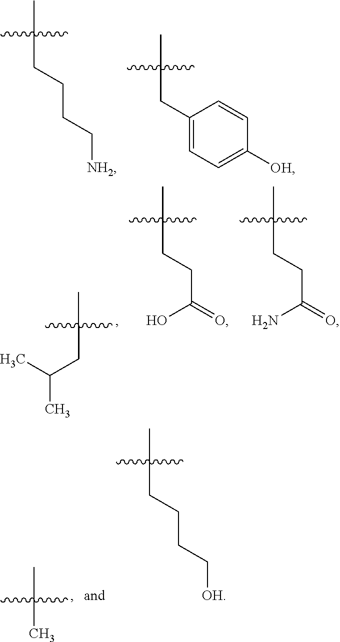

36. The method of any one of claims 13-35, wherein R³ is

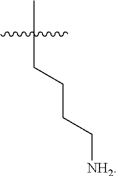 [SMILES: *CCCCN]

37. The method of any one of claims 13-36, wherein the compound has the structure of formula (I-3L):

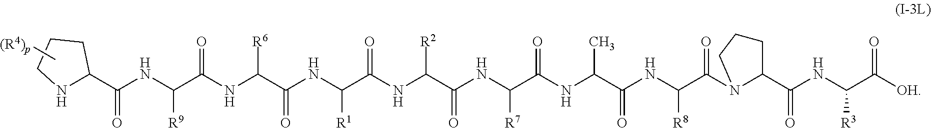

38. The method of any one of claims 13-36, wherein the compound has the structure of formula (I-3D):

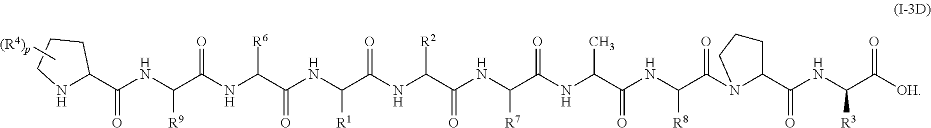

39. The method of any one of claims 13-38, wherein p is 1 or 2; and
R⁴, independently for each occurrence, is selected from substituted or unsubstituted alkyl, —OR^{b}, —CH₂OR^{b}, halo, hydroxyl, and hydroxyalkyl.

40. The method of any one of claims 13-39, wherein p is 1 or 2; and
R⁴, independently for each occurrence, is selected from —CH₃, halo, hydroxyl, and hydroxyalkyl.

41. The method of claim 40, wherein R⁴ is hydroxyl.

42. The method of claim 40, wherein R⁴ is —CH₃.

43. The method of any one of claims 39-42, wherein p is 1.

44. The method of any one of claims 13-43, wherein the compound has the structure of formula (I-4Lg):

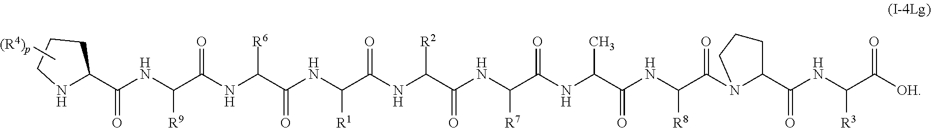

45. The method of any one of claims 13-43, wherein the compound has the structure of formula (I-4La):

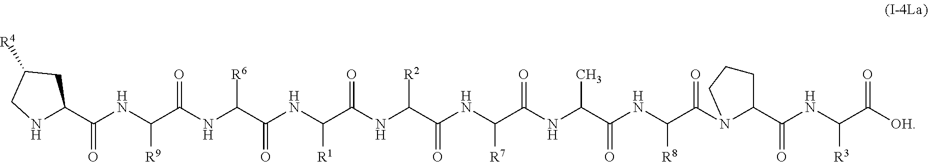

46. The method of any one of claims 13-43, wherein the compound has the structure of formula (I-4Lb):

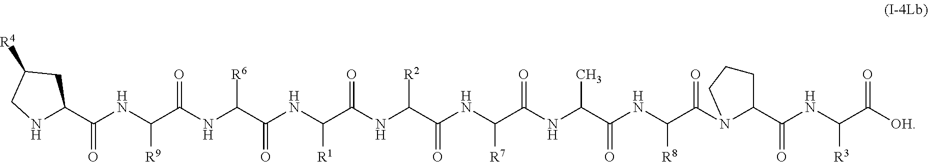

47. The method of any one of claims 13-40, 42, and 43, wherein the compound has the structure of formula (I-4Lc):

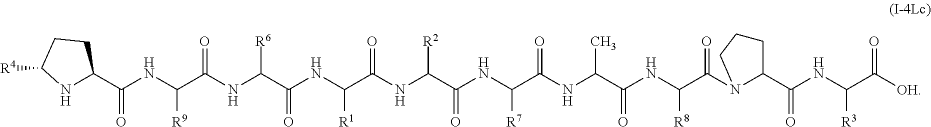

48. The method of any one of claims 13-43, wherein the compound has the structure of formula (I-4Dg):

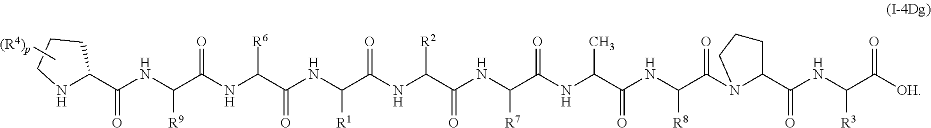

49. The method of any one of claims 13-43 and 48, wherein the compound has the structure of formula (I-4Da):

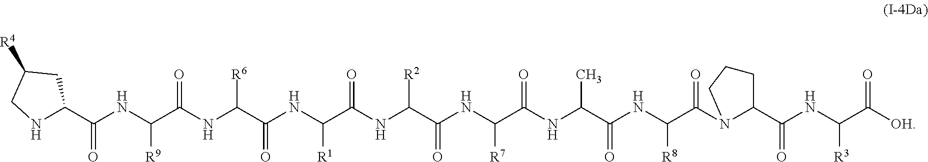

50. The method of any one of claims 13-43 and 48, wherein the compound has the structure of formula (I-4Db):

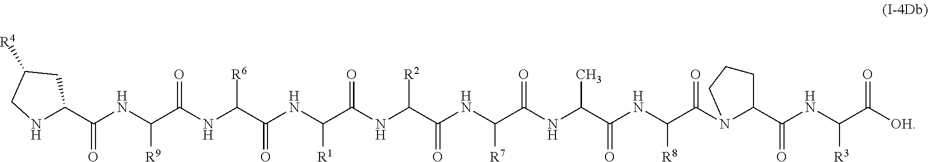

51. The method of any one of claims 13-40, 42, 43, and 48, wherein the compound has the structure of formula (I-4Dc):

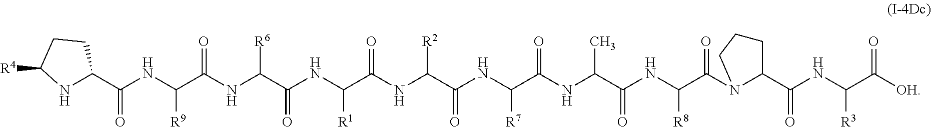

52. The method of any one of claims 13-38, wherein R⁴ is oxo.

53. The method of claim 52, wherein the compound has the structure of formula (I-4Ld):

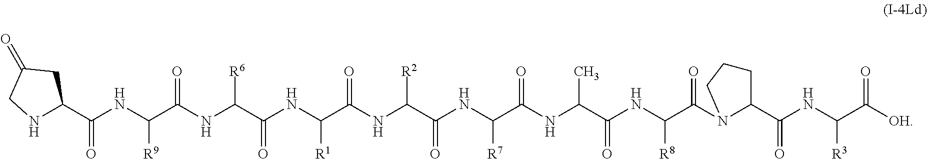

54. The method of claim 52, wherein the compound has the structure of formula (I-4Le):

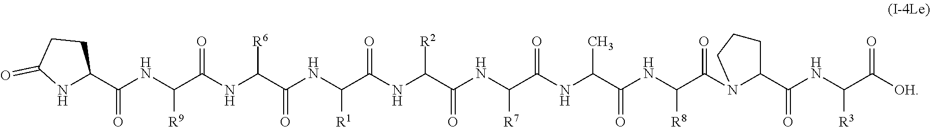

55. The method of claim 52, wherein the compound has the structure of formula (I-4Dd):

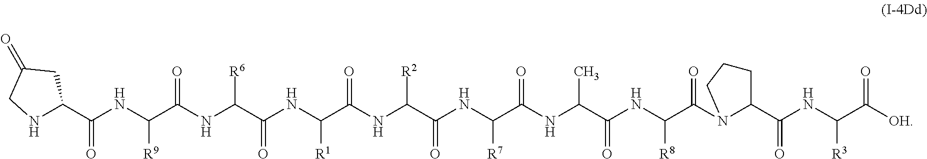

56. The method of claim 52, wherein the compound has the structure of formula (I-4De):

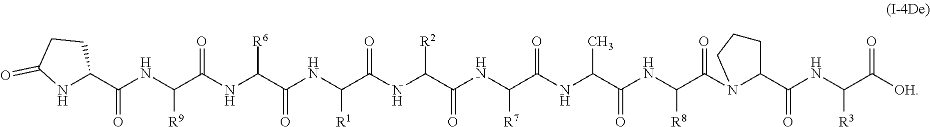

57. The method of any one of claims 1-56, wherein R⁶ is hydrogen or alkyl, wherein the alkyl is optionally substituted with one occurrence of —C(═O)NH₂.

58. The method of any one of claims 1-57, wherein R⁶ is alkyl optionally substituted with one occurrence of —C(═O)NH₂.

59. The method of any one of claims 1-58, wherein R⁶ is —CH₃.

60. The method of any one of claims 1-58, wherein R⁶ is

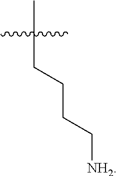 [SMILES: *CCCCN]

61. The method of any one of claims 1-60, wherein the compound has the structure of formula (I-6L):

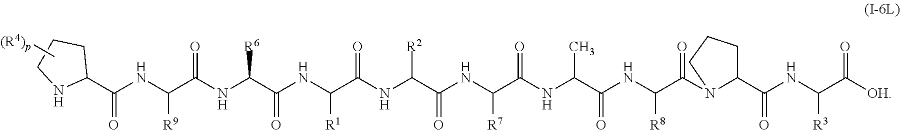

62. The method of any one of claims 1-60, wherein the compound has the structure of formula (I-6D):

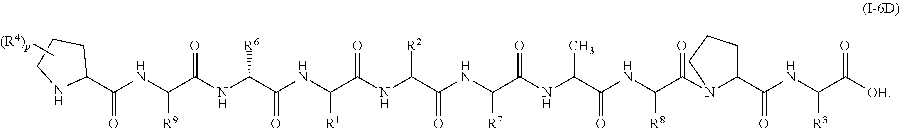

63. The method of any one of claims 1-62, wherein R⁷ is (C₁-C₁₀)alkyl.

64. The method of any one of claims 1-63, wherein R⁷ is

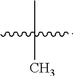

65. The method of any one of claims 1-63, wherein R⁷ is

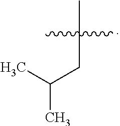 [SMILES: *CC(C)C]

66. The method of any one of claims 1-65, wherein the compound has the structure of formula (I-7L):

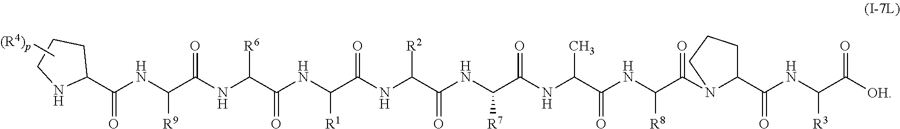

67. The method of any one of claims 1-65, wherein the compound has the structure of formula (I-7D):

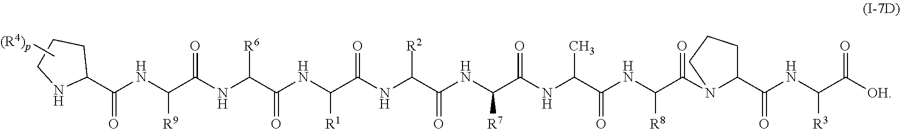

68. The method of any one of claims 1-67, wherein the compound has the structure of formula (I-11L):

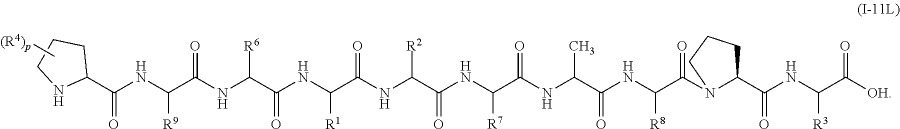

69. The method of any one of claims 1-67, wherein the compound has the structure of formula (I-11 D):

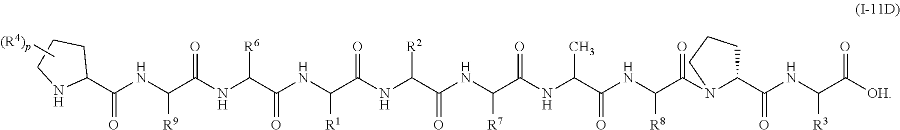

70. The method of any one of claims 1-69, wherein R⁸ is —CH₃ or —H.

71. The method of any one of claims 1-70, wherein R⁸ is —H.

72. The method of any one of claims 1-71, wherein R⁹ is —CH₃ or —H.

73. The method of any one of claims 1-72, wherein R⁹ is —H.

74. The method of claim 13, wherein the compound is selected from the following: 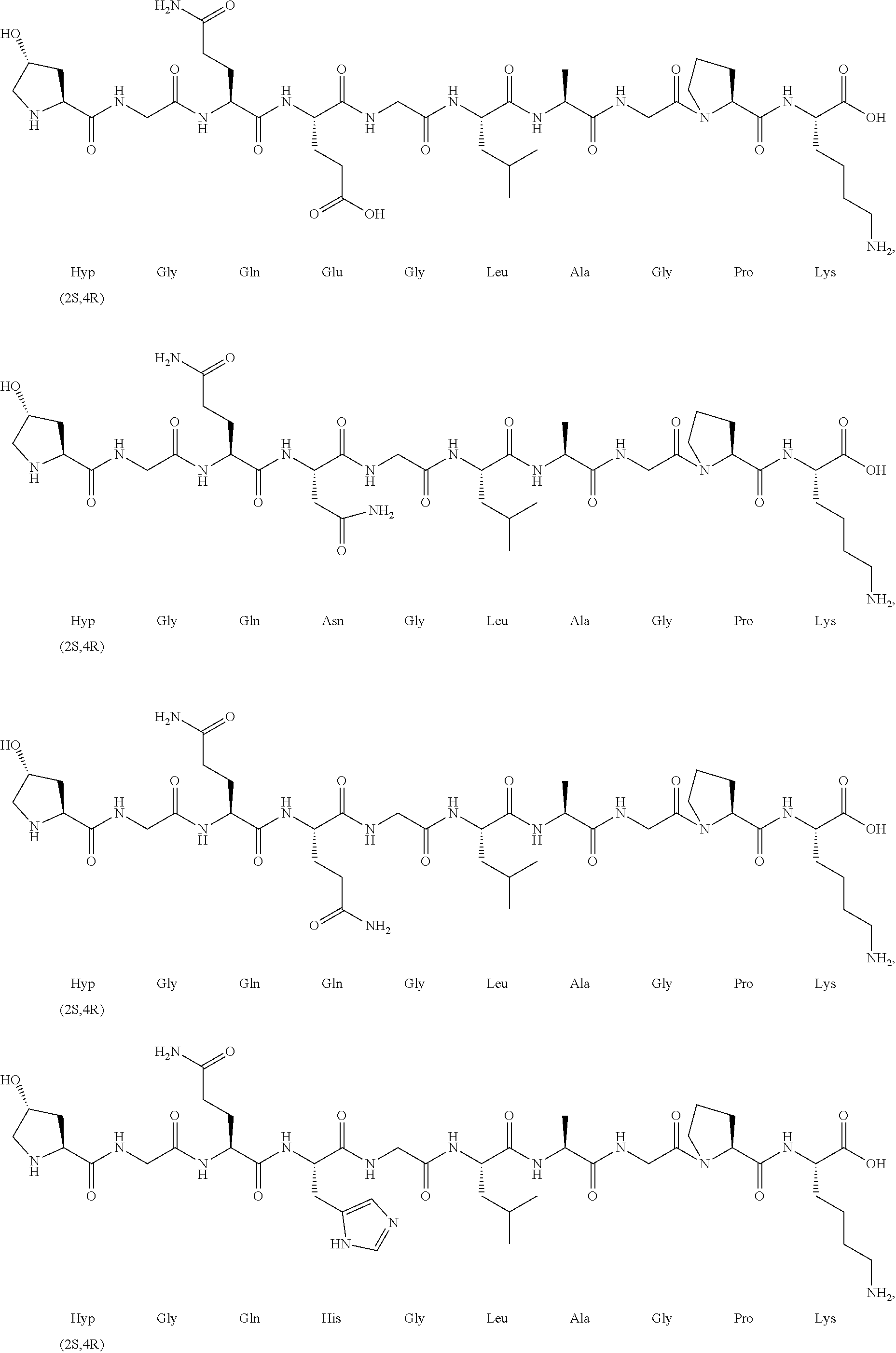 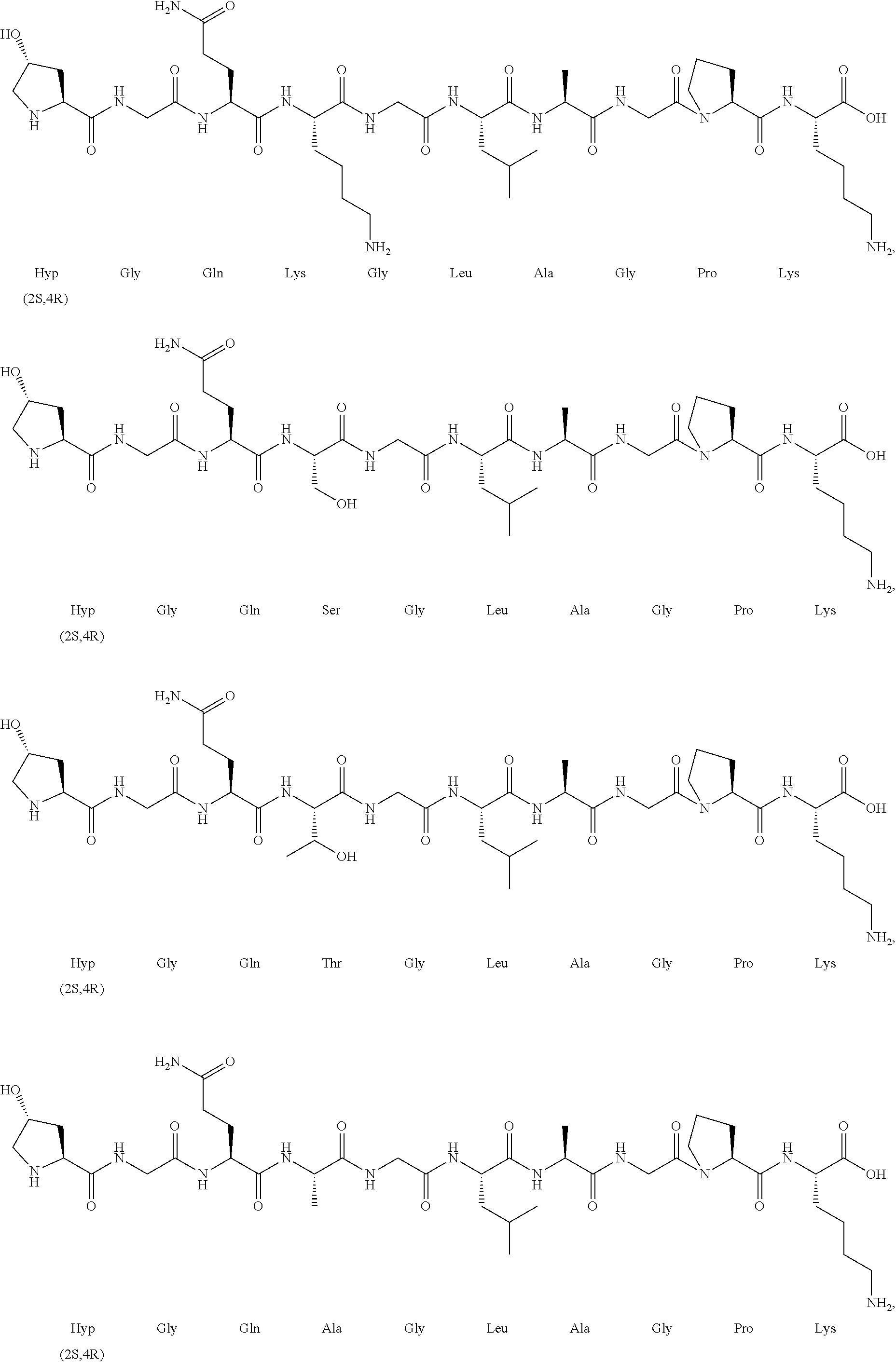 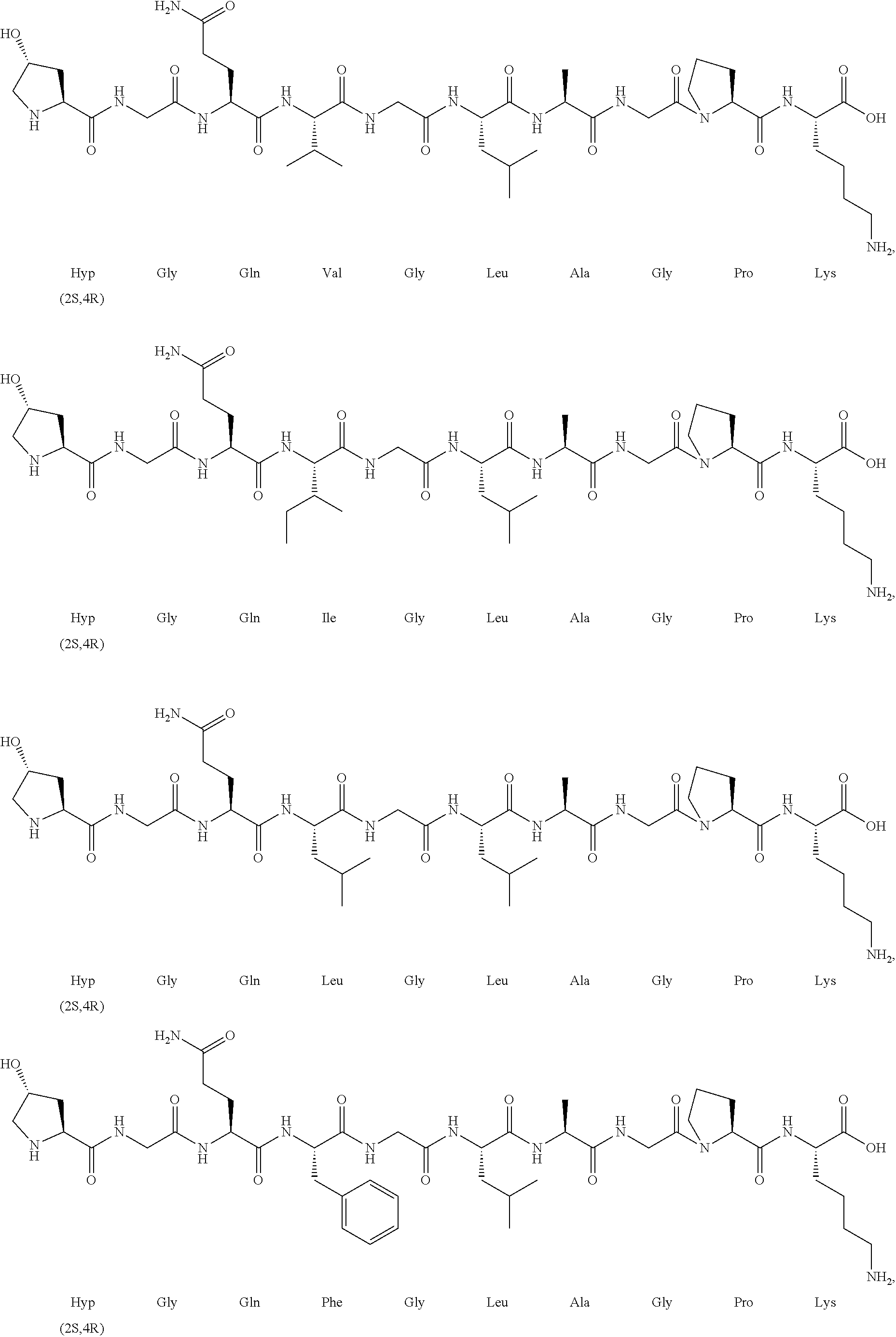 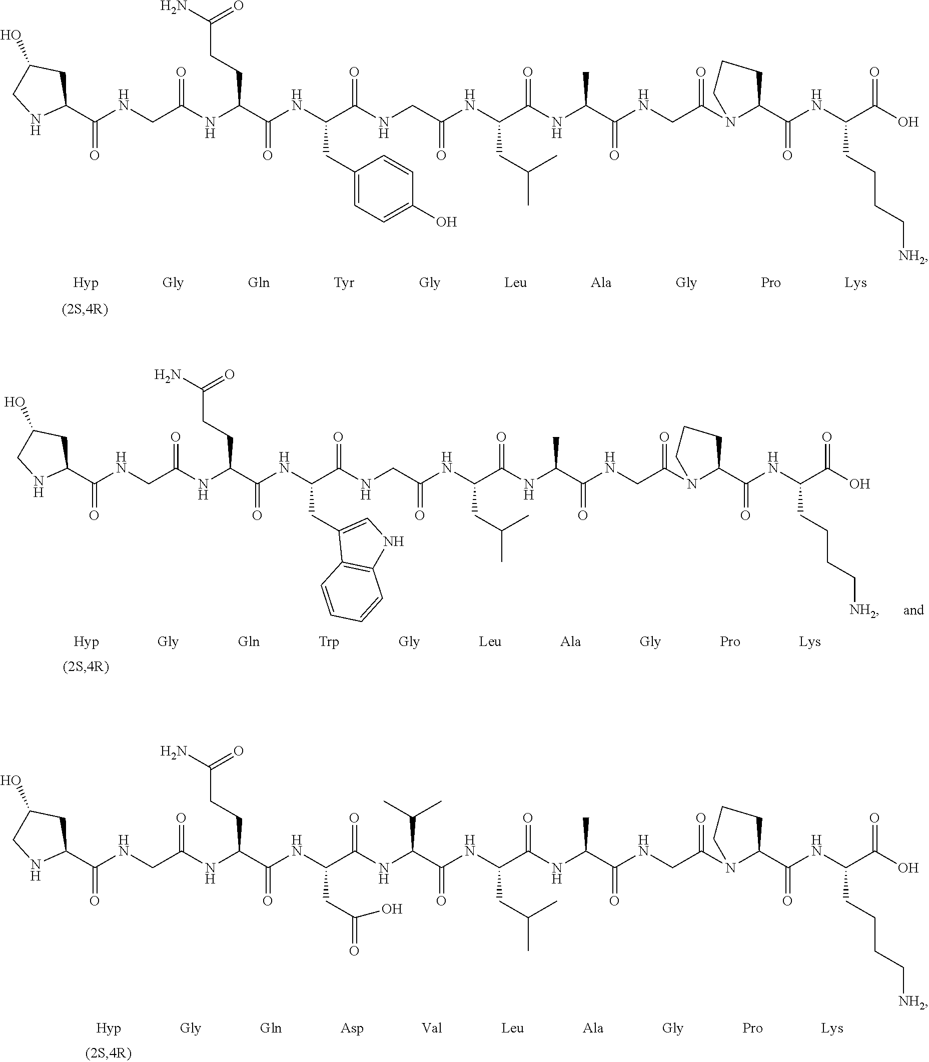
or a pharmaceutically acceptable salt thereof.

75. The method of claim 13, wherein the compound is selected from the following: 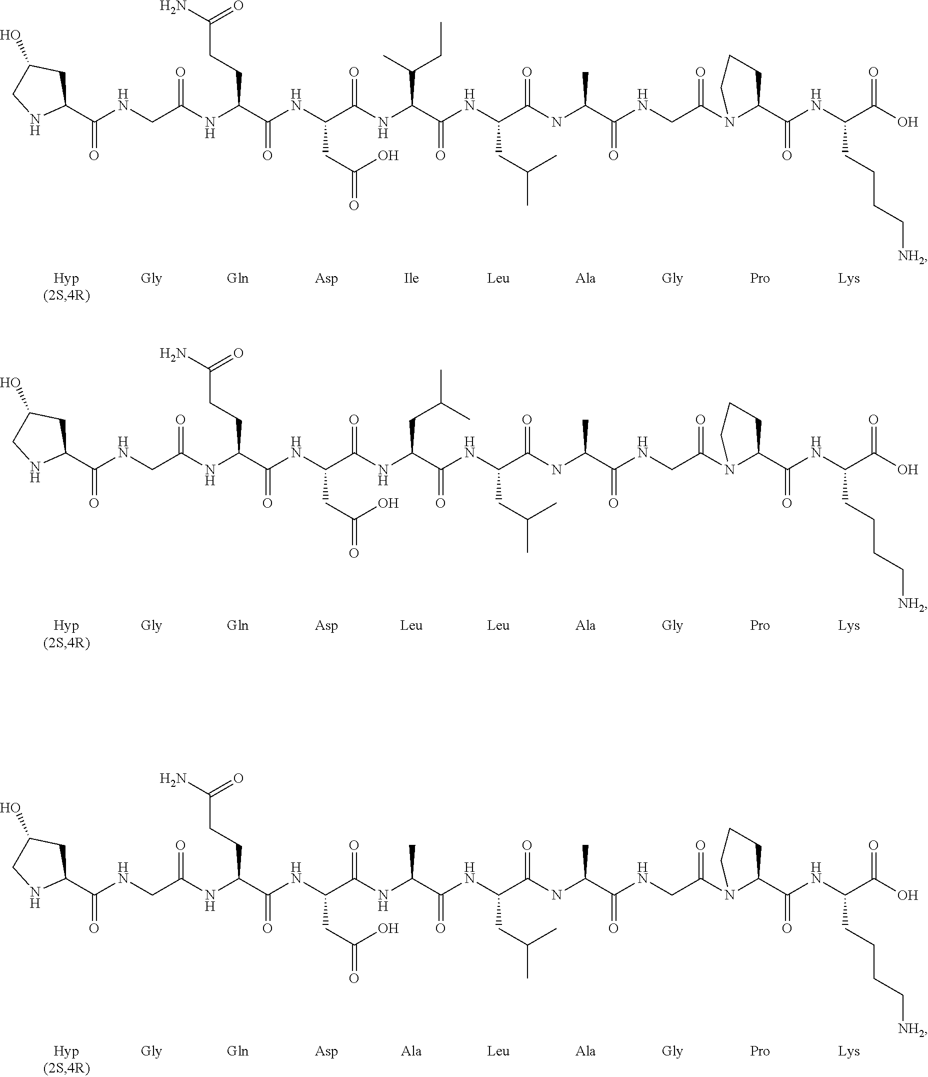 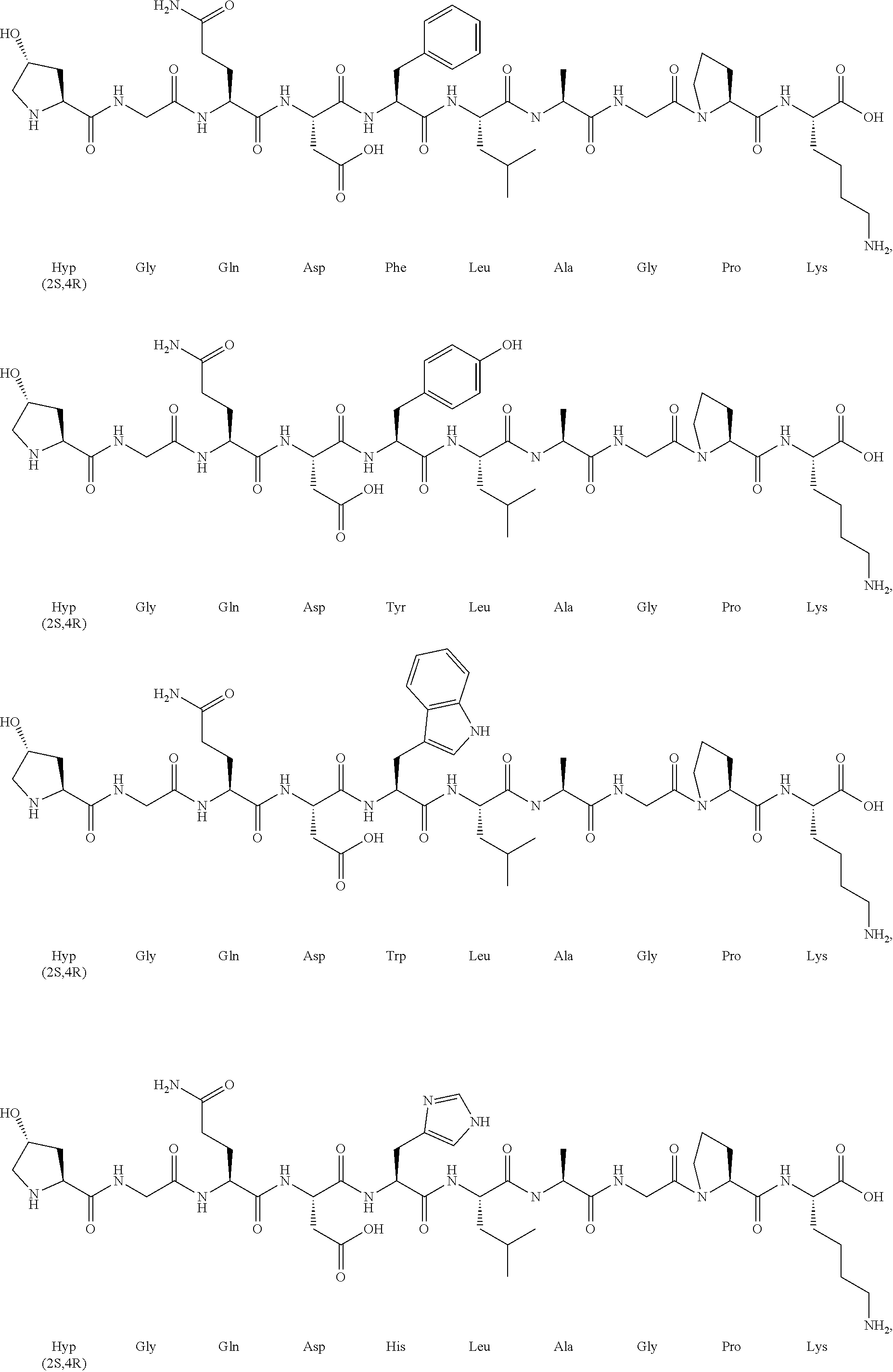 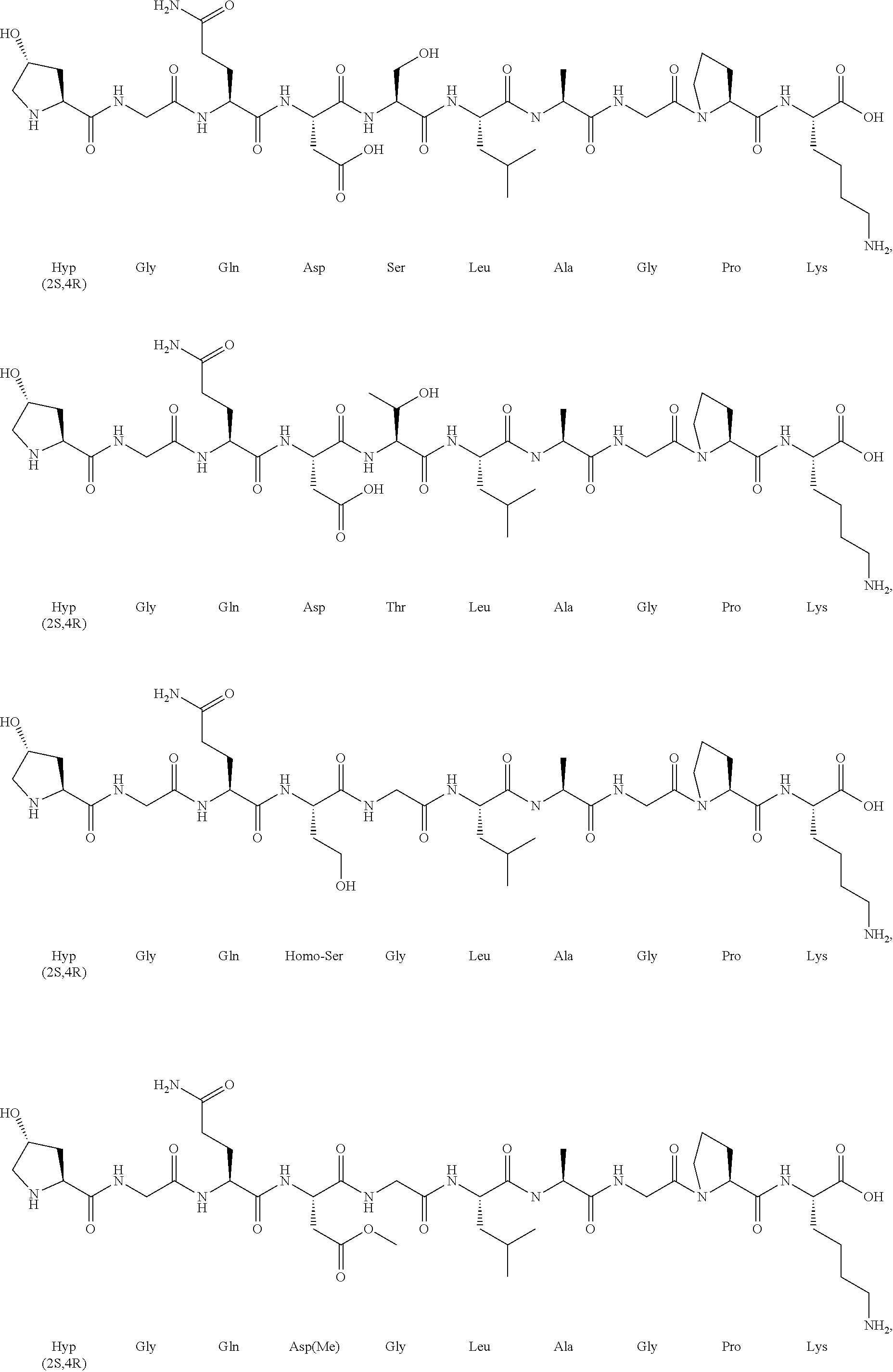 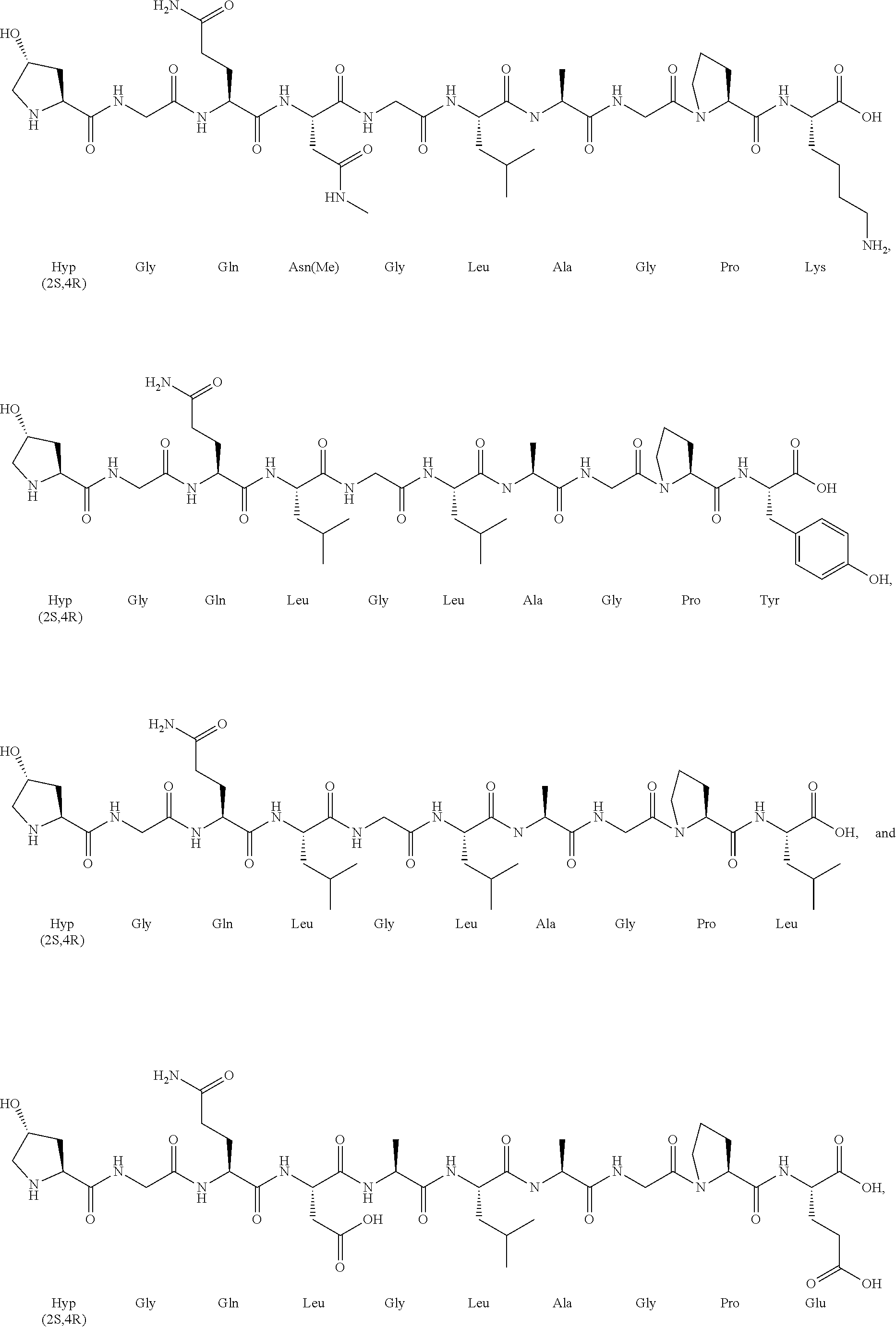
or a pharmaceutically acceptable salt thereof.

76. The method of claim 13, wherein the compound is selected from the following: 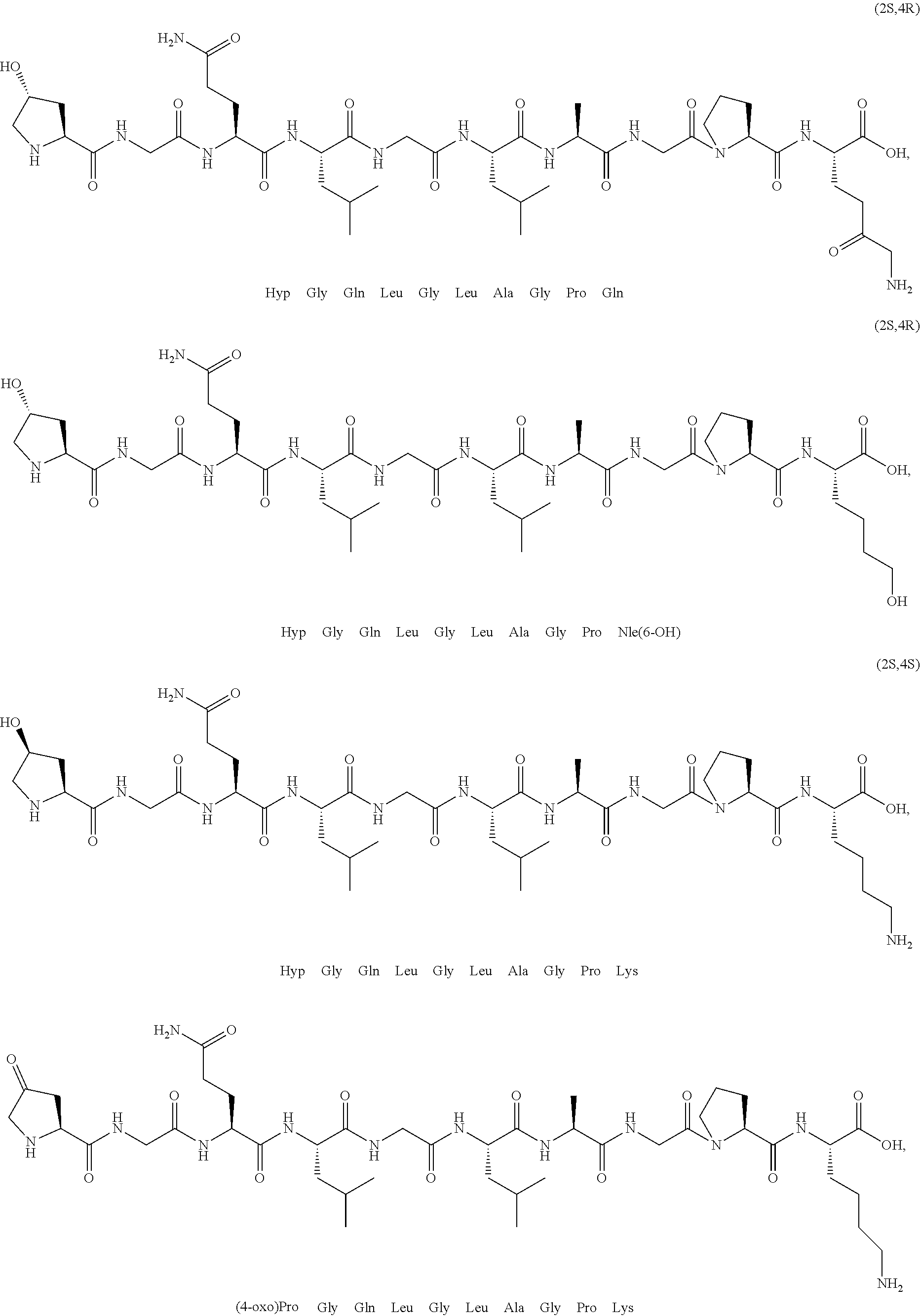 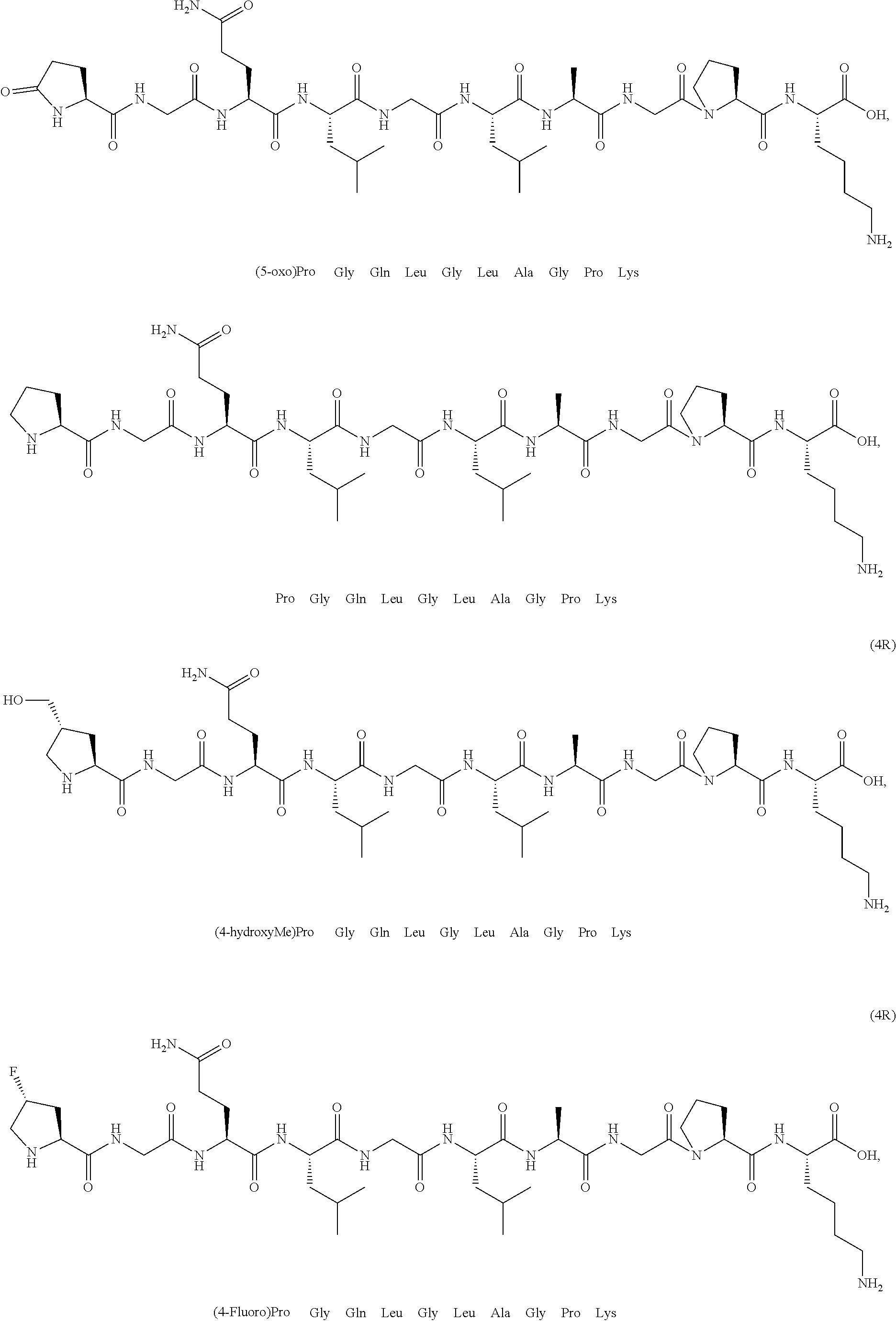 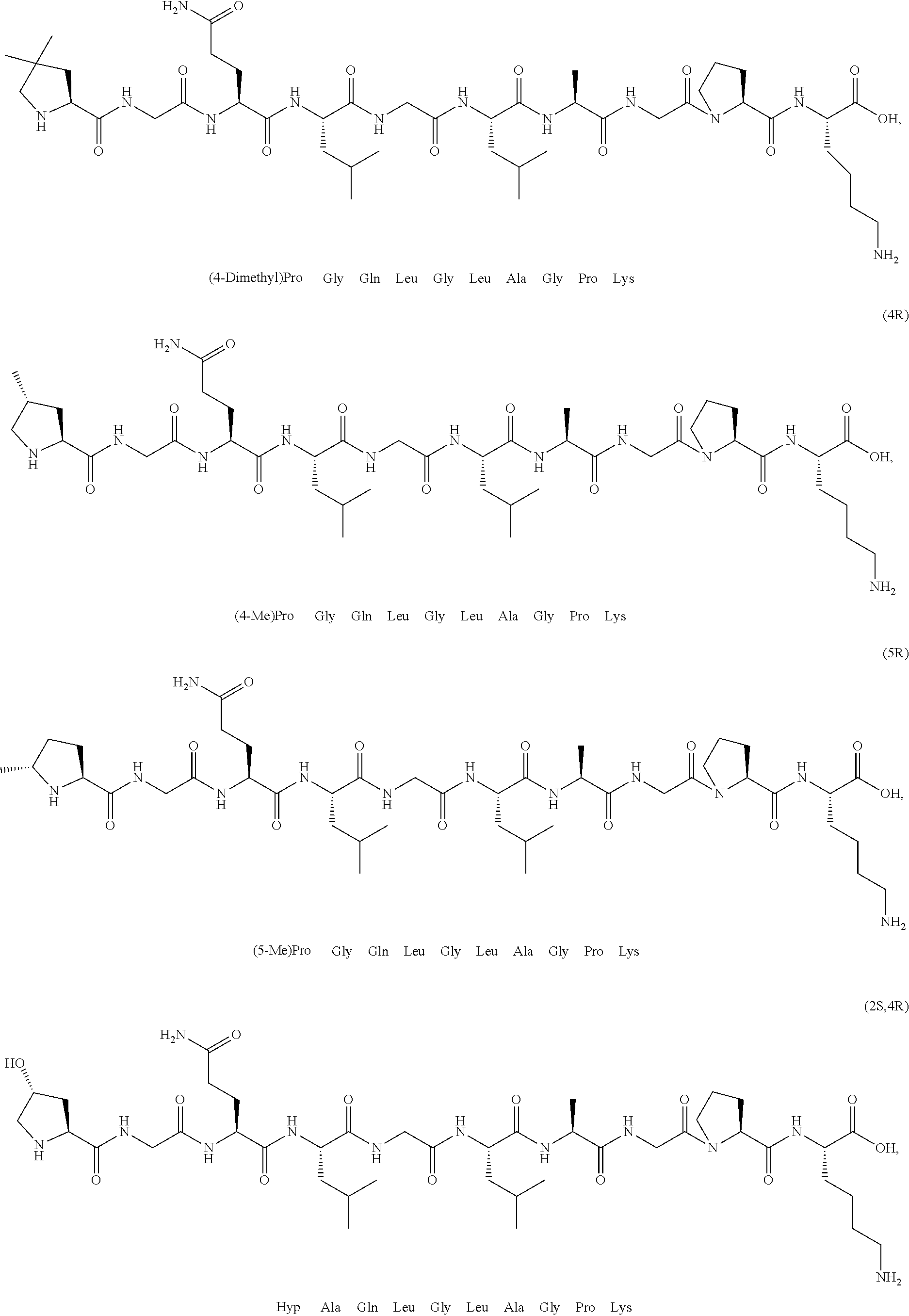 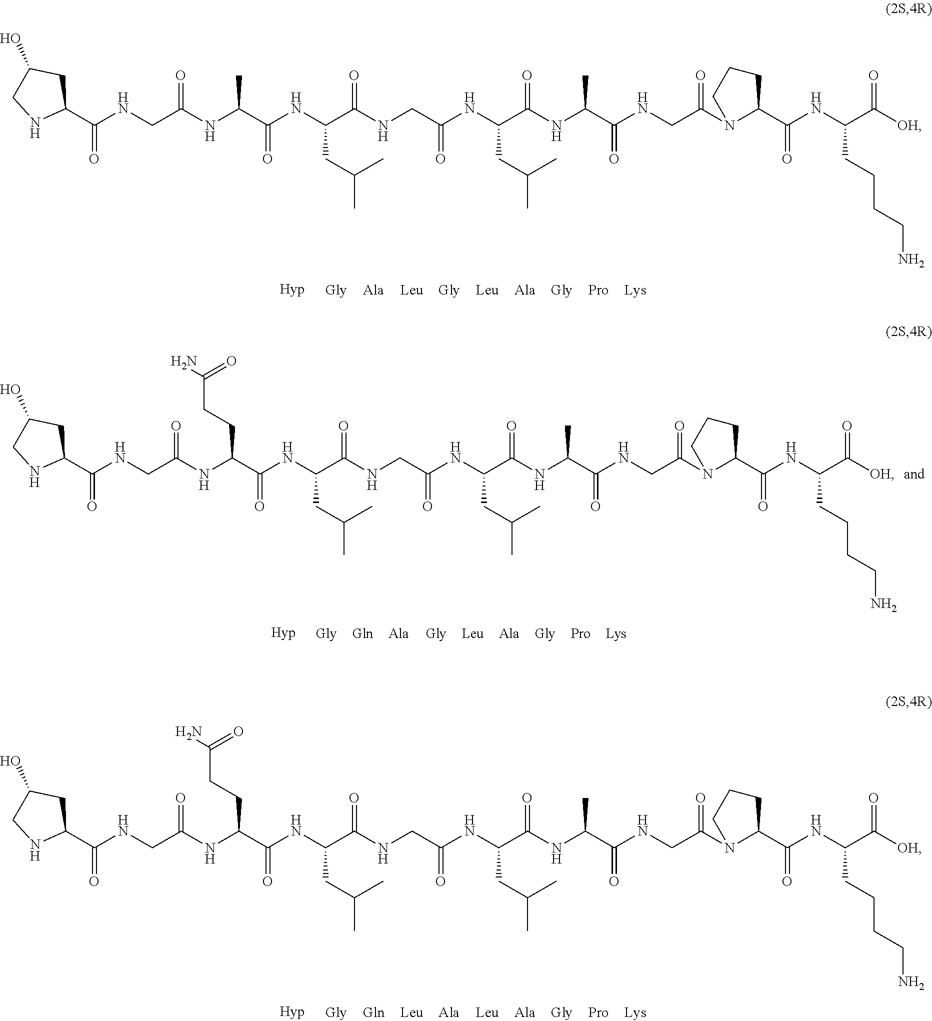 [SMILES: CC(C)C[C@H](NC(=O)CNC(=O)[C@H](CC(C)C)NC(=O)[C@H](C)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O ; CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O ; CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O]
or a pharmaceutically acceptable salt thereof.

77. The method of claim 13, wherein the compound is selected from the following: 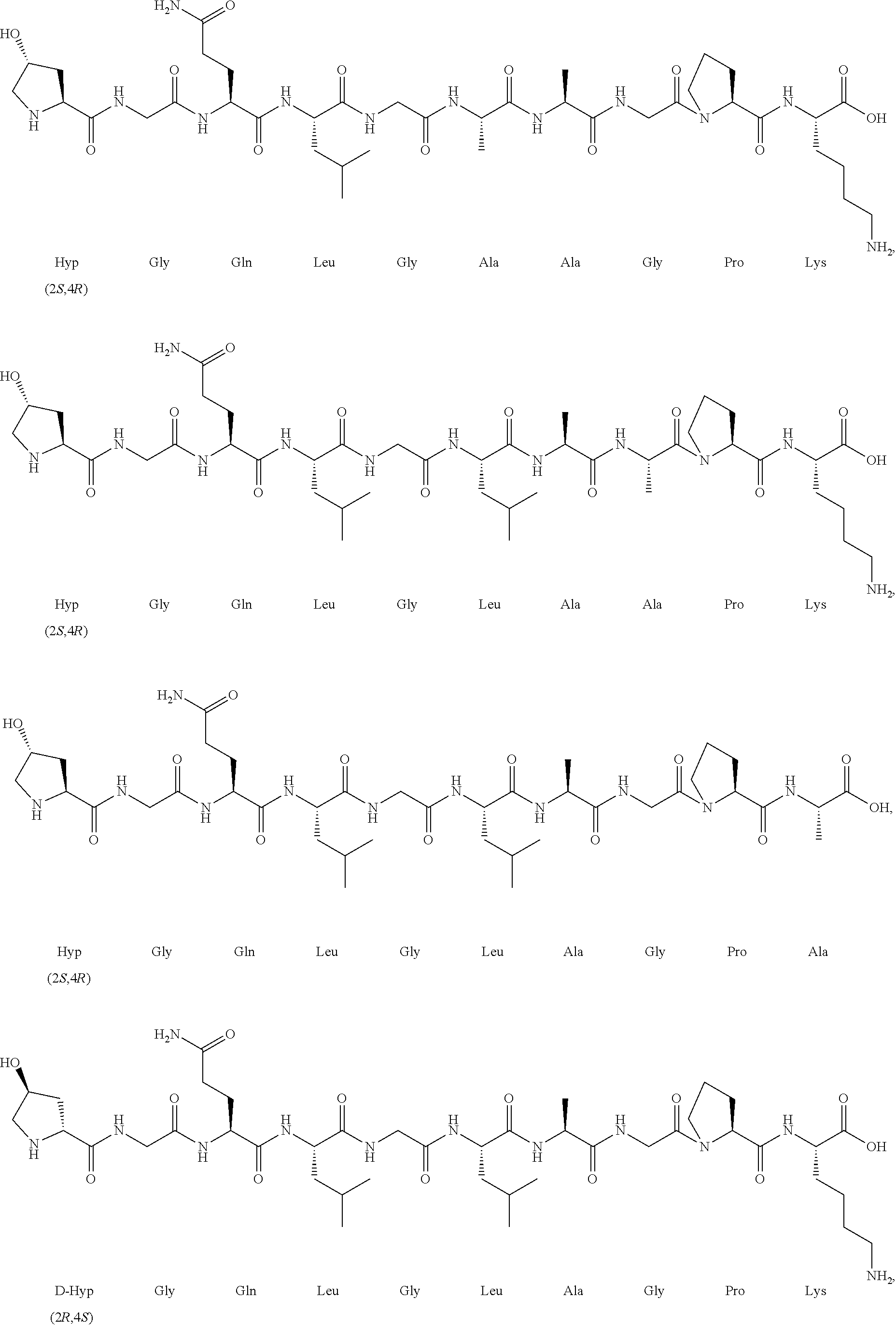 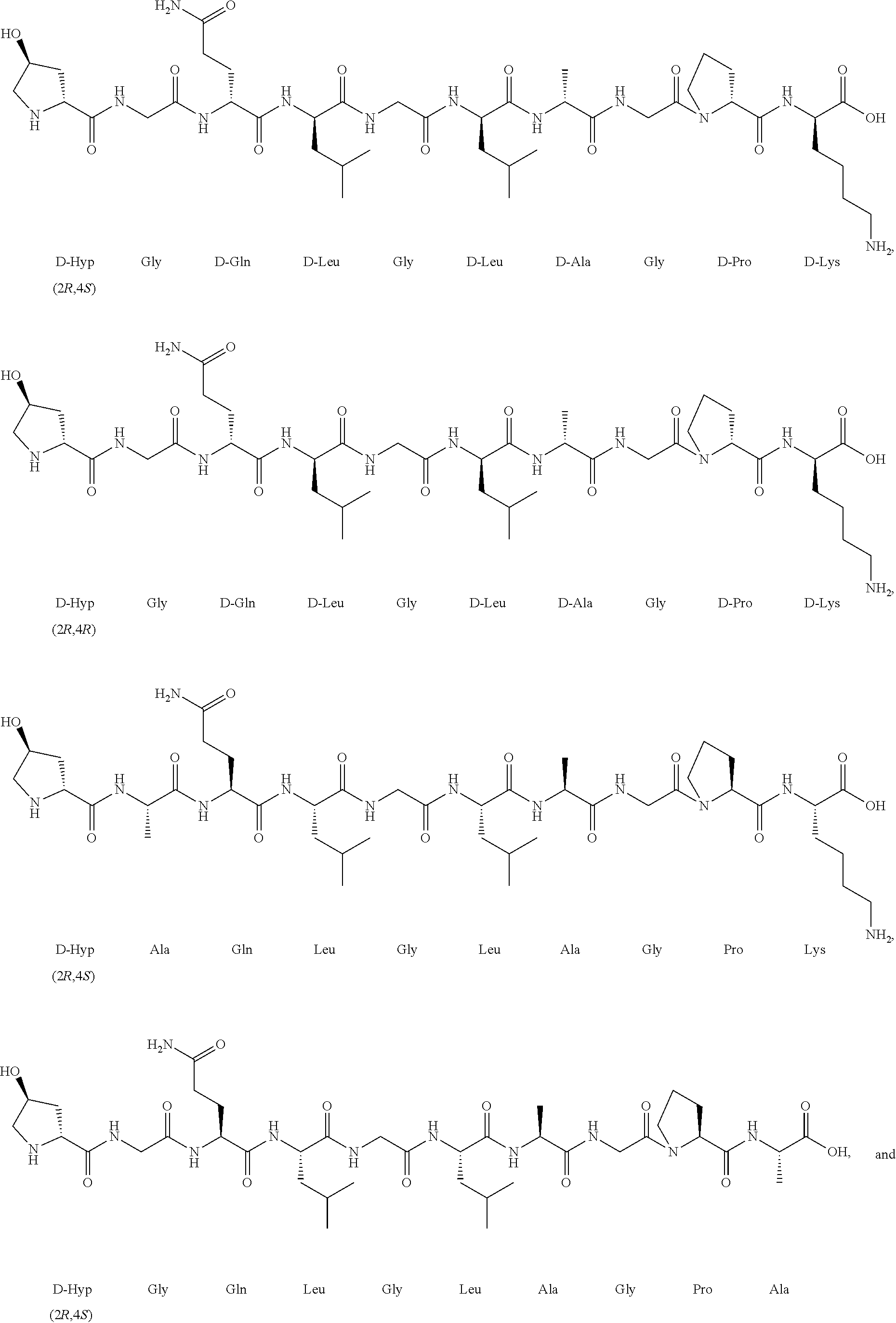 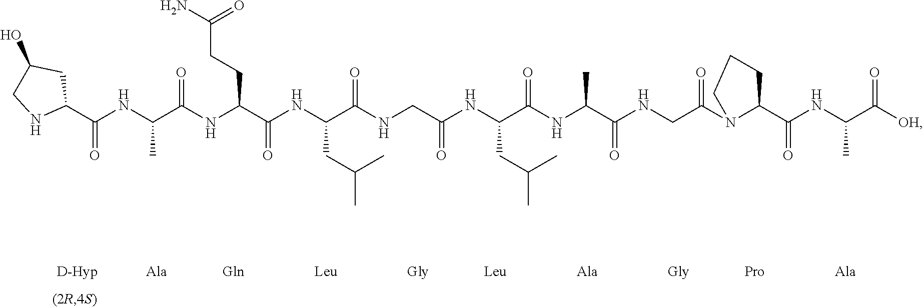 [SMILES: CC(C)C[C@H](NC(=O)CNC(=O)[C@H](CC(C)C)NC(=O)[C@H](CCC(N)=O)NC(=O)[C@H](C)NC(=O)[C@H]1C[C@H](O)CN1)C(=O)N[C@@H](C)C(=O)NCC(=O)N1CCC[C@H]1C(=O)N[C@@H](C)C(=O)O]
or a pharmaceutically acceptable salt thereof.

78. The method of claim 13, wherein the compound is selected from the following: 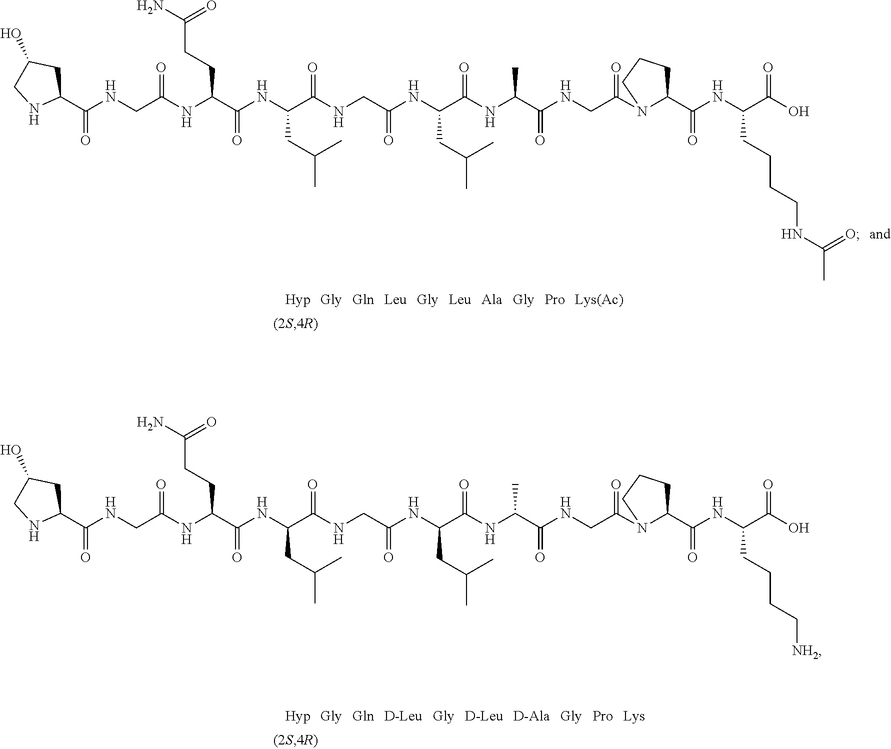 [SMILES: CC(=O)NCCCC[C@H](NC(=O)[C@@H]1CCCN1C(=O)CNC(=O)[C@H](C)NC(=O)[C@H](CC(C)C)NC(=O)CNC(=O)[C@H](CC(C)C)NC(=O)[C@H](CCC(N)=O)NC(=O)CNC(=O)[C@@H]1C[C@@H](O)CN1)C(=O)O ; CC(C)C[C@@H](NC(=O)CNC(=O)[C@@H](CC(C)C)NC(=O)[C@H](CCC(N)=O)NC(=O)CNC(=O)[C@@H]1C[C@@H](O)CN1)C(=O)N[C@H](C)C(=O)NCC(=O)N1CCC[C@H]1C(=O)N[C@@H](CCCCN)C(=O)O]
or a pharmaceutically acceptable salt thereof.

79. The method of any one of claims 1-12, wherein the peptide has an amino acid sequence represented by HyP-Gly-Gln-Xaa-Gly-Leu-Ala-Gly-Pro-Lys;
or a pharmaceutically acceptable salt and/or stereoisomer thereof;
wherein Xaa is selected from Glu, Asn, Gln, His, Lys, Ser, Thr, Ala, Val, Ile, Leu, Phe, Tyr, Trp, homo-Ser, Asp(Me), and Asn(Me).

80. The method of any one of claims 1-12, wherein the peptide has an amino acid sequence represented by HyP-Gly-Gln-Asp-Xbb-Leu-Ala-Gly-Pro-Lys;
or a pharmaceutically acceptable salt and/or stereoisomer thereof;
wherein Xbb is selected from Val, Ile, Leu, Ala, Phe, Tyr, Trp, Ser, Thr, and (N-Me)Gly.

81. The method of any one of claims 1-12, wherein the peptide has an aminoacids sequence represented by HyP-Gly-Gln-Leu-Gly-Leu-Ala-Gly-Pro-Xcc;
or a pharmaceutically acceptable salt and/or stereoisomer thereof;
wherein Xcc is selected from Tyr, Leu, Glu, Gln, Ala, and Nle(6-OH).

82. The method of any one of claims 1-20, wherein the peptide has an amino acid sequence represented by Xdd-Gly-Gln-Leu-Gly-Leu-Ala-Gly-Pro-Lys;
or a pharmaceutically acceptable salt and/or stereoisomer thereof;
wherein Xdd is selected from:

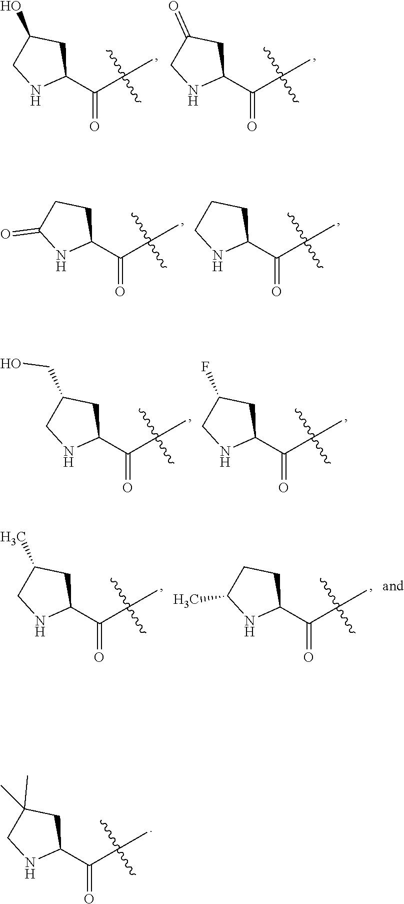

83. The peptide of any one of claims 1-73, wherein at least one, at least two, at least three, at least four, at least five, at least six, or at least seven amino acid residues in the peptide are D-amino acid residues.

84. The method of any one of claims 1-20, wherein the compound is represented by Formula (V): 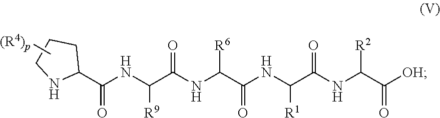
or a pharmaceutically acceptable salt thereof; wherein:
R¹ and R² are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl; and
R⁹ is hydrogen or alkyl.

85. The method of claim 84, wherein:
R¹ and R² are each independently H or substituted or unsubstituted alkyl;
R⁴ for each occurrence is hydroxyl;
p is 1;
R⁶ is alkyl optionally substituted with one occurrence of —C(═O)NH₂; and
R⁹ is hydrogen.

86. The method of claim 84 or 85, wherein R¹ is substituted or unsubstituted alkyl.

87. The method of any one of claims 84-86, wherein R¹ is

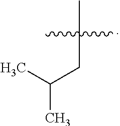 [SMILES: *CC(C)C]

88. The method of any one of claims 84-87, wherein the compound has the structure of formula (V-1L)

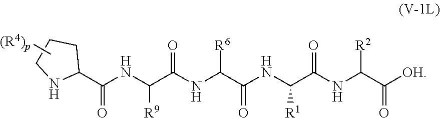

89. The method of any one of claims 84-87, wherein the compound has the structure of formula (V-1D)

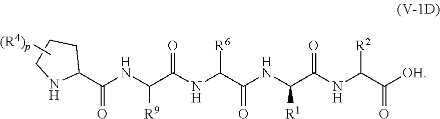

90. The method of any one of claims 84-89, wherein R² is H.

91. The method of any one of claims 84-90, wherein p is 1 and R⁴ is hydroxyl.

92. The method of any one of claims 84-91, wherein the compound has the structure of formula (V-4La):

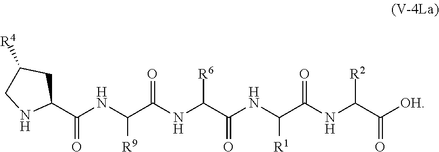

93. The method of any one of claims 84-91, wherein the compound has the structure of formula (V-4Lb):

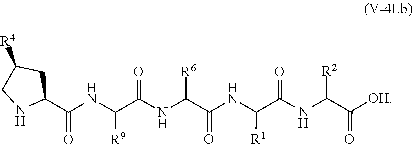

94. The method of any one of claims 84-91, wherein the compound has the structure of formula (V-4Da):

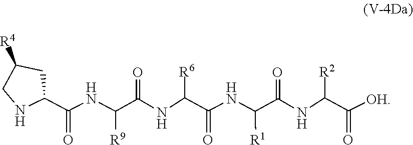

95. The method of any one of claims 84-91, wherein the compound has the structure of formula (V-4Db):

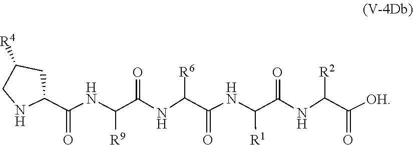

96. The method of any one of claims 84-95, wherein R⁶ is alkyl substituted with one occurrence of —C(═O)NH₂.

97. The method of any one of claims 84-96, wherein R⁶ is

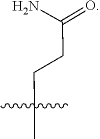 [SMILES: *CCC(N)=O]

98. The method of any one of claims 84-97, wherein the compound has the structure of formula (V-6L):

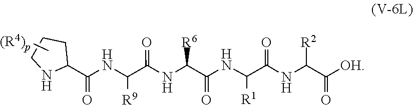

99. The method of any one of claims 84-97, wherein the compound has the structure of formula (V-6D):

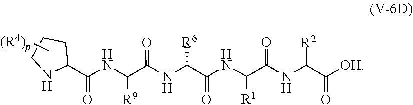

100. The method of any one of claims 84-99, wherein R⁹ is —H.

101. The method of claim 84, wherein the compound is selected from the following: 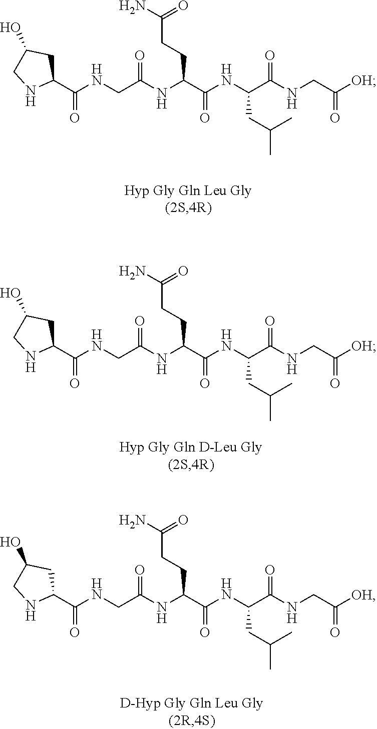 [SMILES: CC(C)C[C@H](NC(=O)[C@H](CCC(N)=O)NC(=O)CNC(=O)[C@@H]1C[C@@H](O)CN1)C(=O)NCC(=O)O ; CC(C)C[C@H](NC(=O)[C@H](CCC(N)=O)NC(=O)CNC(=O)[C@@H]1C[C@@H](O)CN1)C(=O)NCC(=O)O ; CC(C)C[C@H](NC(=O)[C@H](CCC(N)=O)NC(=O)CNC(=O)[C@H]1C[C@H](O)CN1)C(=O)NCC(=O)O]
or a pharmaceutically acceptable salt thereof.

102. The method of claim 84, wherein the compound is selected from the following: 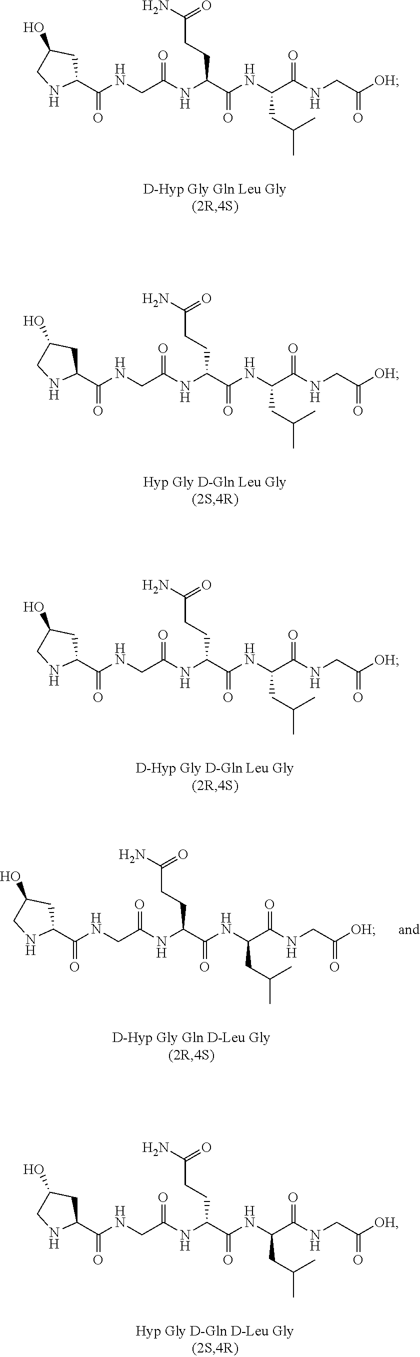 [SMILES: CC(C)C[C@H](NC(=O)[C@H](CCC(N)=O)NC(=O)CNC(=O)[C@H]1C[C@H](O)CN1)C(=O)NCC(=O)O ; CC(C)C[C@H](NC(=O)[C@@H](CCC(N)=O)NC(=O)CNC(=O)[C@@H]1C[C@@H](O)CN1)C(=O)NCC(=O)O ; CC(C)C[C@H](NC(=O)[C@@H](CCC(N)=O)NC(=O)CNC(=O)[C@H]1C[C@H](O)CN1)C(=O)NCC(=O)O ; CC(C)C[C@@H](NC(=O)[C@H](CCC(N)=O)NC(=O)CNC(=O)[C@H]1C[C@H](O)CN1)C(=O)NCC(=O)O ; "and" ; CC(C)C[C@@H](NC(=O)[C@@H](CCC(N)=O)NC(=O)CNC(=O)[C@@H]1C[C@@H](O)CN1)C(=O)NCC(=O)O]
or a pharmaceutically acceptable salt thereof.

103. The method of any one of claims 1-20, wherein the compound is represented by Formula (VI): 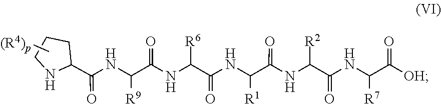
or a pharmaceutically acceptable salt thereof; wherein:
R¹ and R² are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl;
R⁷ is hydrogen or alkyl; and
R⁹ is hydrogen or alkyl.

104. The method of claim 103, wherein:
R¹ and R² are each independently H or substituted or unsubstituted alkyl;
R⁴ for each occurrence is hydroxyl;
p is 1;
R⁶ is alkyl optionally substituted with one occurrence of —C(═O)NH₂; and
R⁹ is hydrogen.

105. The method of claim 103 or 104, wherein R¹ is substituted or unsubstituted alkyl.

106. The method of any one of claims 103-105, wherein R¹ is

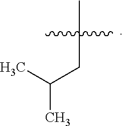 [SMILES: *CC(C)C]

107. The method of any one of claims 103-106, wherein the compound has the structure of formula (VI-1L)

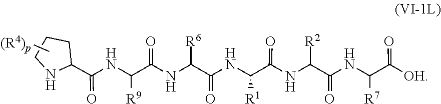

108. The method of any one of claims 103-106, wherein the compound has the structure of formula (VI-1D)

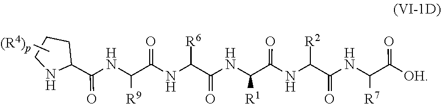

109. The method of any one of claims 103-108, wherein R² is H.

110. The method of any one of claims 103-109, wherein p is 1 and R⁴ is hydroxyl.

111. The method of any one of claims 103-110, wherein the compound has the structure of formula (VI-4La):

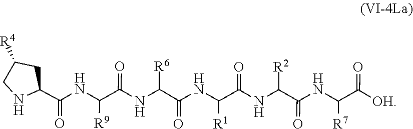

112. The method of any one of claims 103-110, wherein the compound has the structure of formula (VI-4Lb):

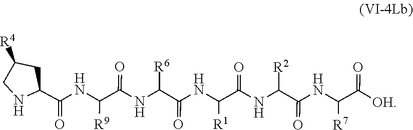

113. The method of any one of claims 103-110, wherein the compound has the structure of formula (VI-4Da):

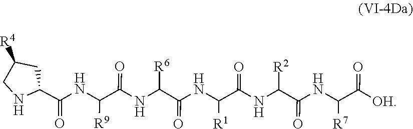

114. The method of any one of claims 103-110, wherein the compound has the structure of formula (VI-4Db):

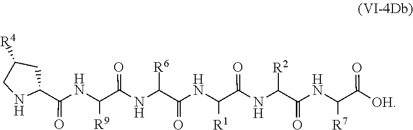

115. The method of any one of claims 103-114, wherein R⁶ is alkyl substituted with one occurrence of —C(═O)NH₂.

116. The method of any one of claims 103-115, wherein R⁶ is

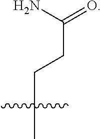 [SMILES: *CCC(N)=O]

117. The method of any one of claims 103-116, wherein the compound has the structure of formula (VI-6L):

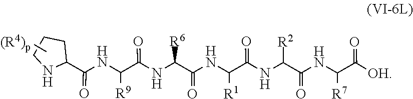

118. The method of any one of claims 103-116, wherein the compound has the structure of formula (VI-6D):

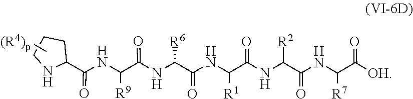

119. The method of any one of claims 103-118, wherein R⁹ is —H.

120. The method of any one of claims 103-119, wherein R⁷ is (C₁-C₁₀)alkyl.

121. The method of any one of claims 103-120, wherein R⁷ is

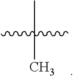

122. The method of any one of claims 103-120, wherein R⁷ is CH₃

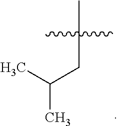 [SMILES: *CC(C)C]

123. The method of any one of claims 103-122, wherein the compound has the structure of formula (VI-7L):

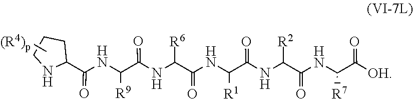

124. The method of any one of claims 103-122, wherein the compound has the structure of formula (VI-7D):

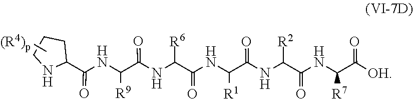

125. The method of claim 103, wherein the compound is selected from the following: 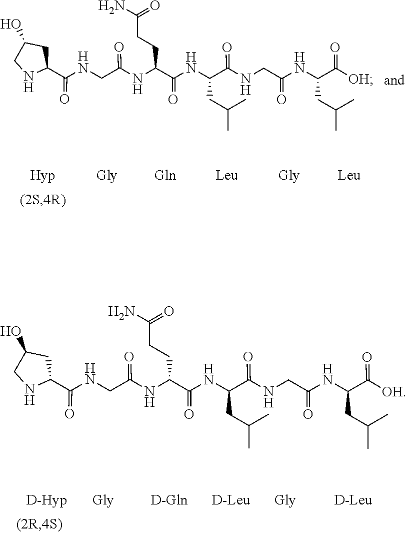 [SMILES: CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)O ; CC(C)C[C@@H](NC(=O)CNC(=O)[C@@H](CC(C)C)NC(=O)[C@@H](CCC(N)=O)NC(=O)CNC(=O)[C@H]1C[C@H](O)CN1)C(=O)O]
or a pharmaceutically acceptable salt thereof.

126. The method of any one of claims 1-20, wherein the compound is represented by Formula (VII): 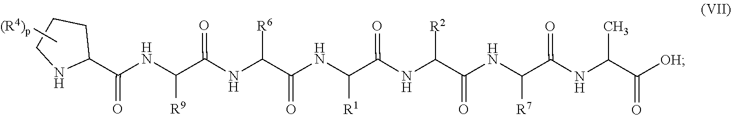
or a pharmaceutically acceptable salt thereof; wherein:
R¹ and R² are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl;
R⁷ is hydrogen or alkyl; and
R⁹ is hydrogen or alkyl.

127. The method of claim 126, wherein:
R¹ and R² are each independently H or substituted or unsubstituted alkyl;
R⁴ for each occurrence is hydroxyl;
p is 1;
R⁶ is alkyl optionally substituted with one occurrence of —C(═O)NH₂; and
R⁹ is hydrogen.

128. The method of claim 126 or 127, wherein R¹ is substituted or unsubstituted alkyl.

129. The method of any one of claims 126-128, wherein R¹ is

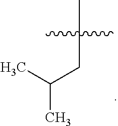 [SMILES: *CC(C)C]

130. The method of any one of claims 126-129, wherein the compound has the structure of formula (VII-1L)

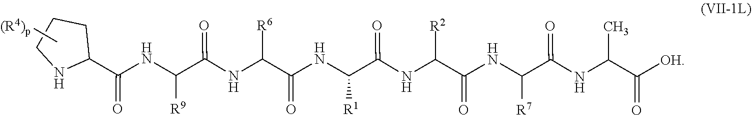

131. The method of any one of claims 126-129, wherein the compound has the structure of formula (VII-1D)

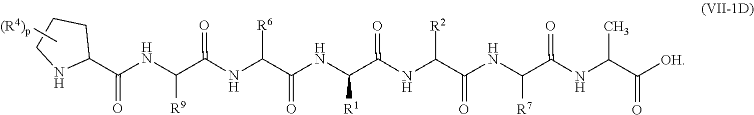

132. The method of any one of claims 126-131, wherein R² is H.

133. The method of any one of claims 126-132, wherein p is 1 and R⁴ is hydroxyl.

134. The method of any one of claims 126-133, wherein the compound has the structure of formula (VII-4La):

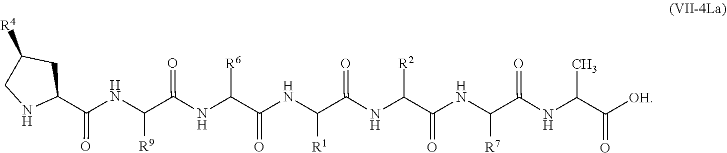

135. The method of any one of claims 126-133, wherein the compound has the structure of formula (VII-4Lb):

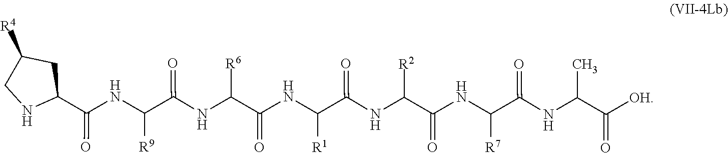

136. The method of any one of claims 126-133, wherein the compound has the structure of formula (VII-4Da):

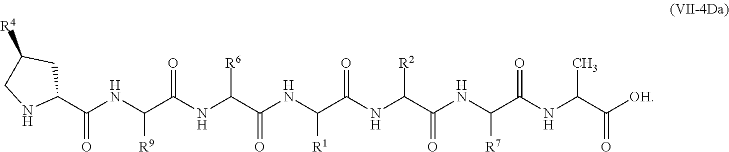

137. The method of any one of claims 126-133, wherein the compound has the structure of formula (VII-4Db):

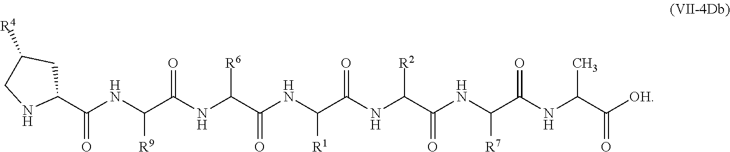

138. The method of any one of claims 126-137, wherein R⁶ is alkyl substituted with one occurrence of —C(═O)NH₂.

139. The method of any one of claims 126-138, wherein R⁶ is

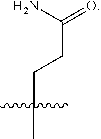 [SMILES: *CCC(N)=O]

140. The method of any one of claims 126-139, wherein the compound has the structure of formula (VII-6L):

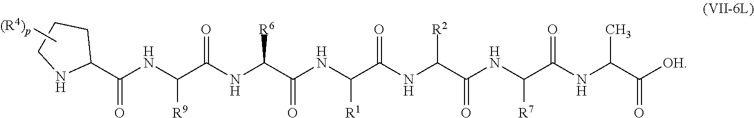

141. The method of any one of claims 126-139, wherein the compound has the structure of formula (VII-6D):

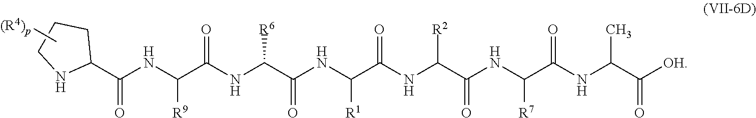

142. The method of any one of claims 126-141, wherein R⁹ is —H.

143. The method of any one of claims 126-142, wherein R⁷ is (C₁-C₁₀)alkyl.

144. The method of any one of claims 126-143, wherein R⁷ is

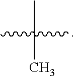

145. The method of any one of claims 126-143, wherein R⁷ is

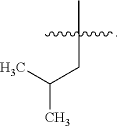 [SMILES: *CC(C)C]

146. The method of any one of claims 126-145, wherein the compound has the structure of formula (VII-7L):

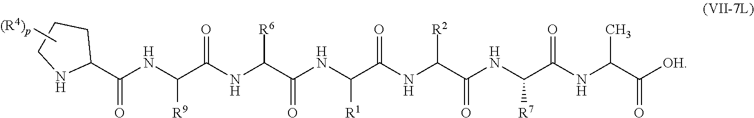

147. The method of any one of claims 126-145, wherein the compound has the structure of formula (VII-7D):

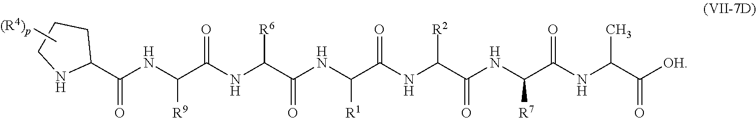

148. The method of any one of claims 126-147, wherein the compound has the structure of formula (VII-10L):

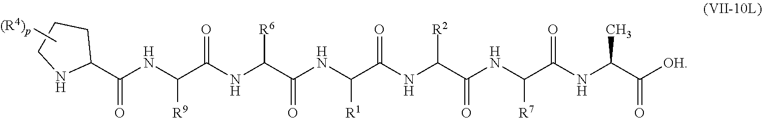

149. The method of any one of claims 126-147, wherein the compound has the structure of formula (VII-10D):

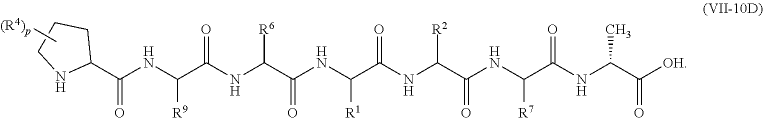

150. The method of claim 126, wherein the compound has the structure: 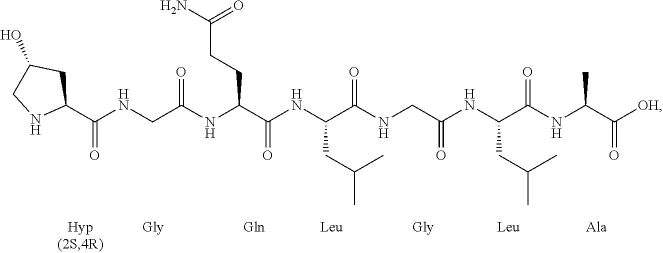 [SMILES: CC(C)C[C@H](NC(=O)CNC(=O)[C@H](CC(C)C)NC(=O)[C@H](CCC(N)=O)NC(=O)CNC(=O)[C@@H]1C[C@@H](O)CN1)C(=O)N[C@@H](C)C(=O)O]
or a pharmaceutically acceptable salt thereof.

151. The method of any one of claims 1-12, wherein the peptide has any one amino acid sequence selected from:
Ala-Gly-Gln-Leu-Gly-Leu-Ala-Gly-Pro-Lys;
Hyp-Gly-Gln-Leu-Gly-Leu-Ala-Gly-Ala-Lys;
HyP-Gly-Gln-Leu-Gly-Leu-Ala;
HyP-Gly-Gln-Glu-Gly-Leu-Gly;
HyP-Gly-Gln-Leu-Gly-Leu;
_{D}-HyP(2R, 4S)-Gly-_{D}-Gln-_{D}-Leu-Gly-_{D}-Leu;
HyP-Gly-Gln-Leu-Gly, HyP-Gly-Gln-_{D}-Leu-Gly; and
_{D}-HyP(2R, 4S)-Gly-Gln-Leu-Gly;
or a pharmaceutically acceptable salt thereof.

152. The method of any one of claims 1-12, wherein the compound is represented by Formula (IX): 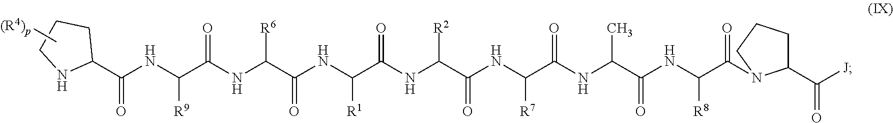
or a pharmaceutically acceptable salt thereof;
wherein:
R¹ and R² are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl;
R⁴, independently for each occurrence, is selected from substituted or unsubstituted alkyl, aryl, arylalkyl, heterocyclyl, oxo, —OR^{b}, —CH₂OR^{b}, halo, hydroxyl, and hydroxyalkyl;
R^{b} is substituted or unsubstituted alkyl, aryl, arylalkyl, or heterocyclyl;
p is 0, 1, or 2;
R⁶ is hydrogen or substituted or unsubstituted alkyl;
R⁷, R⁸, and R⁹ are each independently hydrogen or alkyl;
J is OH or —NR^{x}R^{y}; and
R^{x} and R^{y} are each independently selected from H, optionally substituted alkyl, optionally substituted alkoxylalkyl, or R^{x} and R^{y} taken together with the intervening nitrogen atom form a ring.

153. The method of claim 152, wherein:
R¹ and R² are each independently H or substituted or unsubstituted alkyl;
R⁴ for each occurrence is hydroxyl;
p is 1;
R⁶ is alkyl optionally substituted with one occurrence of —C(═O)NH₂; and
R⁹ is hydrogen.

154. The method of claim 152 or 153, wherein R¹ is substituted or unsubstituted alkyl.

155. The method of any one of claims 152-154, wherein R¹ is

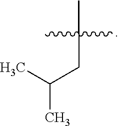 [SMILES: *CC(C)C]

156. The method of any one of claims 152-155, wherein the compound has the structure of formula (IX-1L)

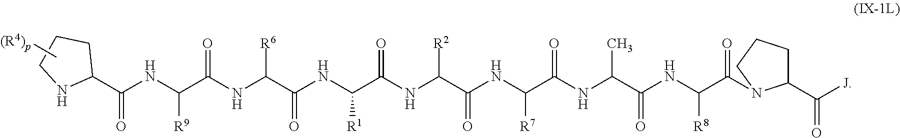

157. The method of any one of claims 152-155, wherein the compound has the structure of formula (IX-1D)

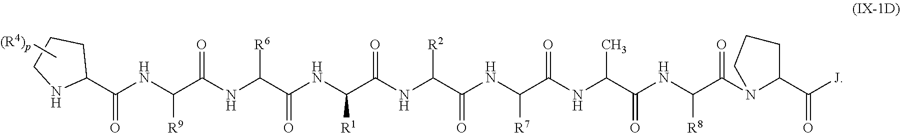

158. The method of any one of claims 152-157, wherein R² is H.

159. The method of any one of claims 152-158, wherein p is 1 and R⁴ is hydroxyl.

160. The method of any one of claims 152-159, wherein the compound has the structure of formula (IX-4La):

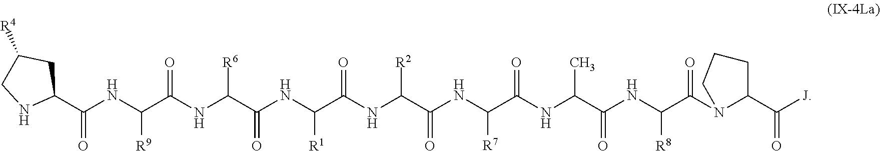

161. The method of any one of claims 152-159, wherein the compound has the structure of formula (IX-4Lb):

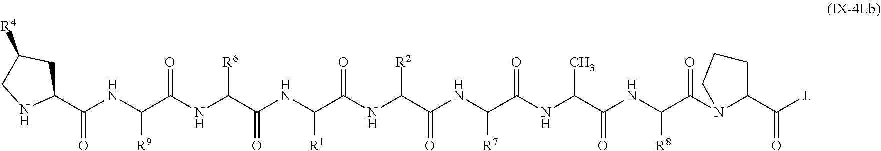

162. The method of any one of claims 152-159, wherein the compound has the structure of formula (IX-4Da):

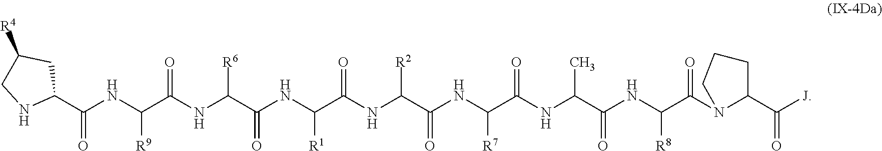

163. The method of any one of claims 152-159, wherein the compound has the structure of formula (IX-4Db):

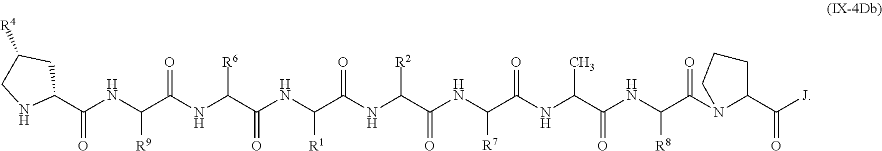

164. The method of any one of claims 152-163, wherein R⁶ is alkyl substituted with one occurrence of —C(═O)NH₂.

165. The method of any one of claims 152-164, wherein R⁶ is

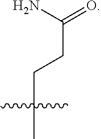 [SMILES: *CCC(N)=O]

166. The method of any one of claims 152-165, wherein the compound has the structure of formula (IX-6L):

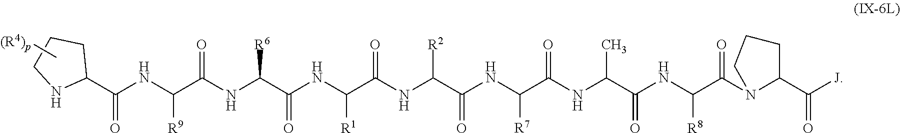

167. The method of any one of claims 152-165, wherein the compound has the structure of formula (IX-6D):

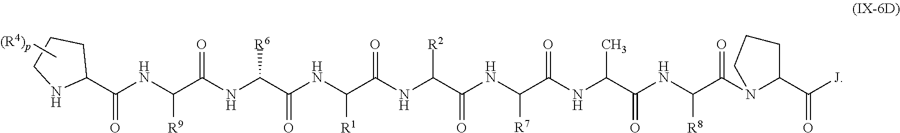

168. The method of any one of claims 152-167, wherein R⁹ is —H.

169. The method of any one of claims 152-168, wherein R⁷ is (C₁-C₁₀)alkyl.

170. The method of any one of claims 152-169, wherein R⁷ is

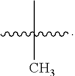

171. The method of any one of claims 152-169, wherein R⁷ is

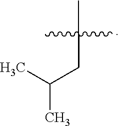 [SMILES: *CC(C)C]

172. The method of any one of claims 152-171, wherein the compound has the structure of formula (IX-7L):

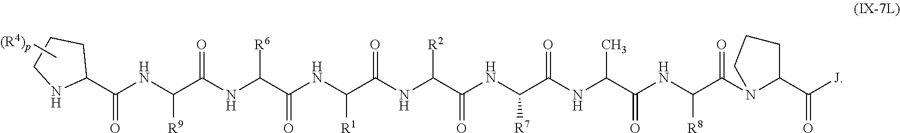

173. The method of any one of claims 152-171, wherein the compound has the structure of formula (IX-7D):

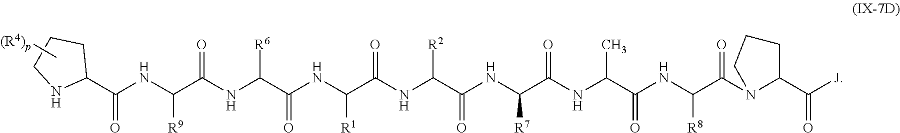

174. The method of any one of claims 152-173, wherein the compound has the structure of formula (IX-10L):

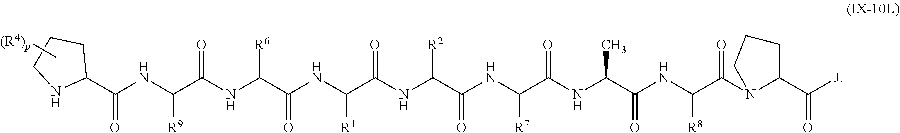

175. The method of any one of claims 152-173, wherein the compound has the structure of formula (IX-10D):

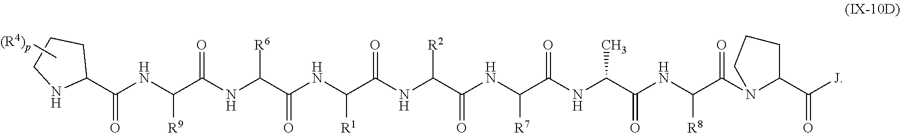

176. The method of any one of claims 152-175, wherein the compound has the structure of formula (IX-11L):

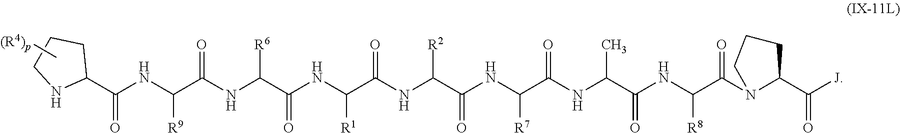

177. The method of any one of claims 152-175, wherein the compound has the structure of formula (IX-11D):

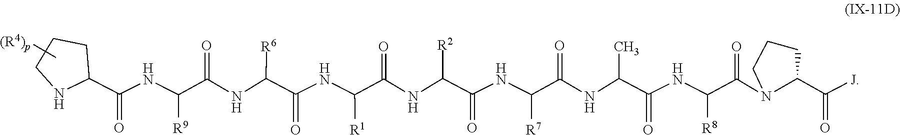

178. The method of any one of claims 152-177, wherein R⁸ is —CH₃ or —H.

179. The method of any one of claims 152-178, wherein R⁸ is —H.

180. The method of any one of claims 152-179, wherein J is OH.

181. The method of any one of claims 152-179, wherein J is —NR^{x}R^{y}.

182. The method of claim 181, wherein R^{x} and R^{y} are each independently alkyl.

183. The method of claim 181, wherein R^{x} and R^{y} taken together with the intervening nitrogen atom form a ring.

184. The method of any one of claims 1-12, wherein the compound is represented by Formula (X-am): 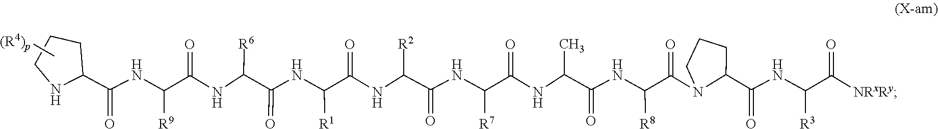
or a pharmaceutically acceptable salt thereof;
wherein:
R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl;
R⁴, independently for each occurrence, is selected from substituted or unsubstituted alkyl, aryl, arylalkyl, heterocyclyl, oxo, —OR^{b}, —CH₂OR^{b}, halo, hydroxyl, and hydroxyalkyl;
R^{b} is substituted or unsubstituted alkyl, aryl, arylalkyl, or heterocyclyl;
p is 0, 1, or 2;
R⁶ is hydrogen or substituted or unsubstituted alkyl;
R⁷, R⁸, and R⁹ are each independently hydrogen or alkyl;
J is OH or —NR^{x}R^{y}; and
R^{x} and R^{y} are each independently selected from H, optionally substituted alkyl, optionally substituted alkoxylalkyl, or R^{x} and R^{y} taken together with the intervening nitrogen atom form a ring.

185. The method of claim 184, wherein:
R¹, R², and R³ are each independently H or substituted or unsubstituted alkyl, arylalkyl, or heterocyclylalkyl;
R⁴, independently for each occurrence, is selected from substituted or unsubstituted alkyl, oxo, hydroxyl, —OR^{b}, hydroxyalkyl, —CH₂OR^{b}, and halo;
R^{b} is substituted or unsubstituted alkyl, aryl, arylalkyl, or heterocyclyl;
R⁶ is hydrogen or substituted or unsubstituted alkyl; and
R⁷, R⁸, and R⁹ are each independently hydrogen or alkyl.

186. The method of claim 184 or 185, wherein, where indicated, alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl is unsubstituted or is substituted with one or more substituents selected from halo, haloalkyl, oxo, —CN, —NO₂, ═N—OH, —N₃, —R^{a}, —OR^{a}, —SR^{a}, —N(R^{a})₂, —N(R^{a})₃⁺, ═NR^{a}, —NHC(═O)R^{c}, —C(═O)R^{c}, —C(═O)N(R^{a})₂, —S(═O)₂R^{c}, —OS(═O)₂OR^{a}, —S(═O)₂OR^{a}, —S(═O)₂N(R^{a})₂, —S(═O)R^{c}, —OP(═O)(OR^{a})₂, -(alkylene)-C(═O)R^{c}, —C(═S)R^{c}, —C(═O)OR^{a}, -(alkylene)-C(═O)OR^{a}, —C(═S)OR^{a}, —C(═O)SR^{a}, —C(═S)SR^{a}, -(alkylene)-C(═O)N(R^{a})₂, —C(═S)N(R^{a})₂, and —C(—NR^{a})N(R^{a})₂; and
R^{a}, independently for each occurrence, is hydrogen, or substituted or unsubstituted alkyl, aryl, arylalkyl, heterocyclyl, heterocyclylalkyl, cycloalkyl, or (cycloalkyl)alkyl; and
R^{c}, independently for each occurrence, is substituted or unsubstituted alkyl, aryl, arylalkyl, heterocyclyl, heterocyclylalkyl, cycloalkyl, or (cycloalkyl)alkyl.

187. The method of any one of claims 184-177, wherein, where indicated, alkyl, alkoxy, haloalkyl, alkenyl, alkynyl, aryl, arylalkyl, arylalkenyl, arylalkynyl, heteroalkyl, cycloalkyl, heterocyclyl, or heterocyclylalkyl is unsubstituted or is substituted with one or more substituents selected from halo, haloalkyl, oxo, —R^{a}, —OR^{a}, —N(R^{a})₂, —N(R^{a})₃⁺, —NHC(═O)R^{c}, —C(═O)R^{c}, —C(═O)N(R^{a})₂, —C(═O)OR^{a}, -(alkylene)-C(═O)OR^{a}, and -(alkylene)-C(═O)N(R^{a})₂; and
R^{a}, independently for each occurrence, is hydrogen, or substituted or unsubstituted alkyl, aryl, arylalkyl, heterocyclyl, heterocyclylalkyl, cycloalkyl, or (cycloalkyl)alkyl; and
R^{c}, independently for each occurrence, is substituted or unsubstituted alkyl, aryl, arylalkyl, heterocyclyl, heterocyclylalkyl, cycloalkyl, or (cycloalkyl)alkyl.

188. The method of claim 186 or 187, wherein R^{a}, independently for each occurrence, is hydrogen, alkyl, aryl, arylalkyl, heterocyclyl, or heterocyclylalkyl; and
R^{c}, independently for each occurrence, is alkyl, aryl, arylalkyl, heterocyclyl, or heterocyclylalkyl.

189. The method of any one of claims 184-188, wherein the compound has the structure of formula (X-am-10L):

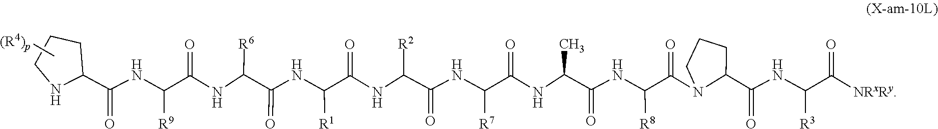

190. The method of any one of claims 184-188, wherein the compound has the structure of formula (X-am-10D):

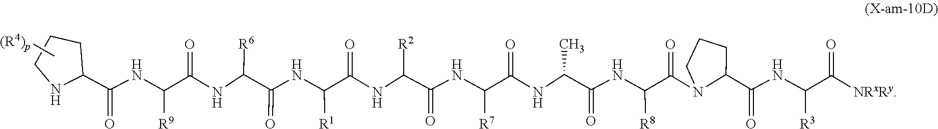

191. The method of any one of claims 184-190, wherein R¹ is substituted or unsubstituted (C₂-C₁₀)haloalkyl.

192. The method of any one of claims 184-190, wherein R¹ is substituted or unsubstituted alkyl, arylalkyl, or heterocyclylalkyl.

193. The method of any one of claims 184-190, wherein R¹ is selected from substituted or unsubstituted alkyl, 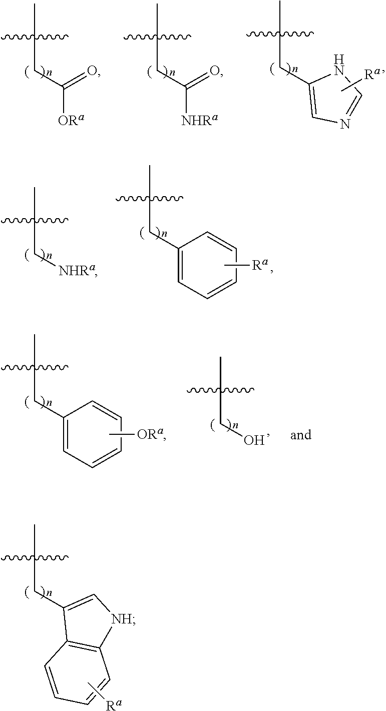
R^{a} is hydrogen or alkyl; and
n is an integer from 1 to 10, preferably 1-5, more preferably 1-3.

194. The method of any one of claims 184-190, wherein R¹ is selected from

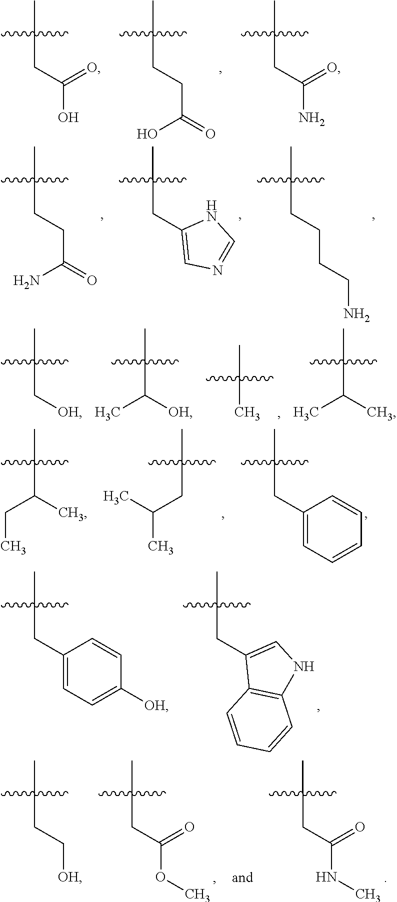

195. The method of any one of claims 184-190, wherein R¹ is

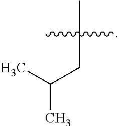 [SMILES: *CC(C)C]

196. The method of any one of claims 184-190, wherein R¹ is

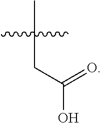 [SMILES: *CC(=O)O]

197. The method of any one of claims 184-196, wherein the compound has the structure of formula (X-am-1L)

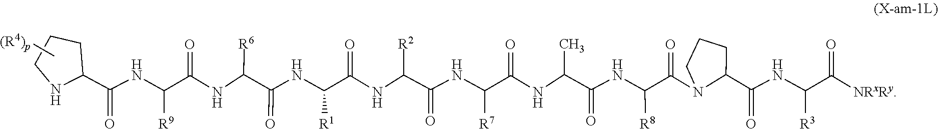

198. The method of any one of claims 184-196, wherein the compound has the structure of formula (X-am-1D)

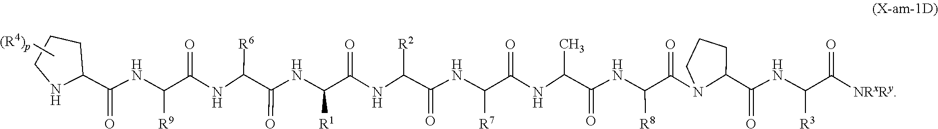

199. The method of any one of claims 184-198, wherein R² is substituted or unsubstituted (C₂-C₁₀)haloalkyl.

200. The method of any one of claims 184-198, wherein R² is H or substituted or unsubstituted alkyl, arylalkyl, or heterocyclylalkyl.

201. The method of any one of claims 184-198, wherein R² is selected from hydrogen, substituted or unsubstituted alkyl, 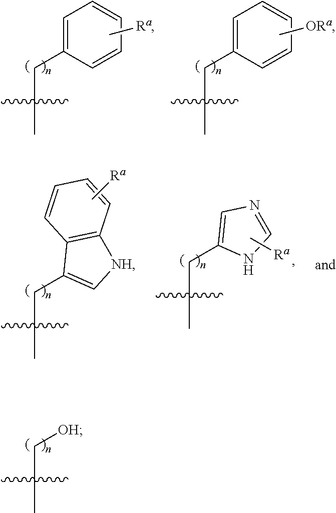
R^{a} is hydrogen or alkyl; and
n is an integer from 1 to 10, preferably 1-5, more preferably 1-3.

202. The method of any one of claims 184-198, wherein R² is selected from

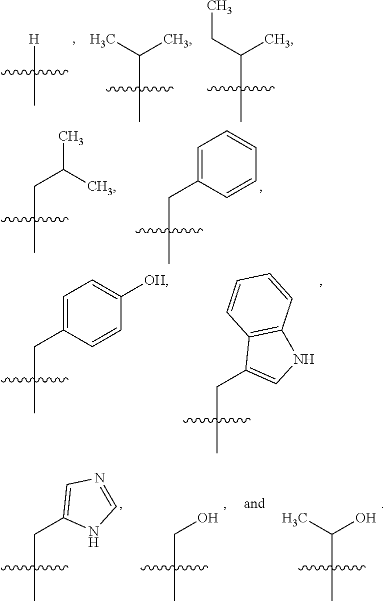

203. The method of any one of claims 184-198, wherein R² is hydrogen.

204. The method of any one of claims 184-203, wherein the compound has the structure of formula (X-am-2L):

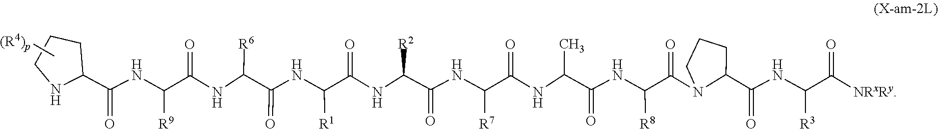

205. The method of any one of claims 184-203, wherein the compound has the structure of formula (X-am-2D):

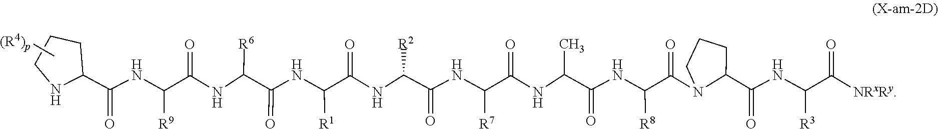

206. The method of any one of claims 184-205, wherein R³ is substituted or unsubstituted (C₂-C₁₀)haloalkyl.

207. The method of any one of claims 184-205, wherein R³ is substituted or unsubstituted alkyl or arylalkyl.

208. The method of any one of claims 184-205 wherein R³ is selected from substituted or unsubstituted alkyl, 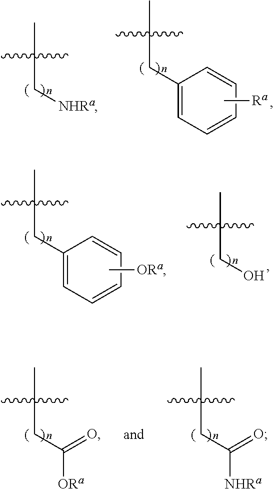
R^{a} is hydrogen or alkyl; and
n is an integer from 1 to 10, preferably 1-5, more preferably 1-3.

209. The method of any one of claims 184-205, wherein R³ is selected from

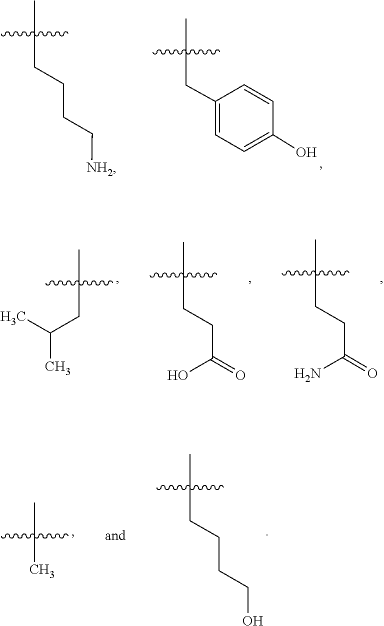

210. The method of any one of claims 184-205, wherein R³ is

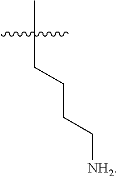 [SMILES: *CCCCN]

211. The method of any one of claims 184-210, wherein the compound has the structure of formula (X-am-3L):

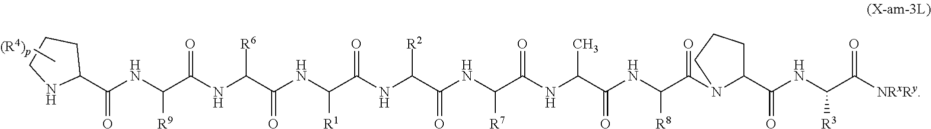

212. The method of any one of claims 184-210, wherein the compound has the structure of formula (X-am-3D):

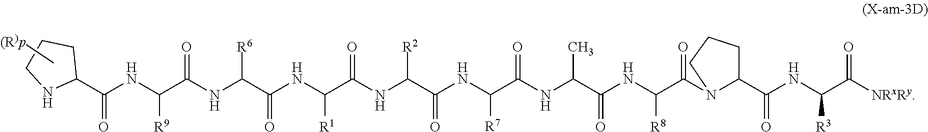

213. The method of any one of claims 184-212, wherein p is 1 or 2; and
R⁴, independently for each occurrence, is selected from substituted or unsubstituted alkyl, —OR^{b}, —CH₂OR^{b}, halo, hydroxyl, and hydroxyalkyl.

214. The method of any one of claims 184-212, wherein p is 1 or 2; and
R⁴, independently for each occurrence, is selected from —CH₃, halo, hydroxyl, and hydroxyalkyl.

215. The method of claim 214, wherein R⁴ is hydroxyl.

216. The method of claim 214, wherein R⁴ is —CH₃.

217. The method of any one of claims 213-216, wherein p is 1.

218. The method of any one of claims 184-217, wherein the compound has the structure of formula (X-am-4Lg):

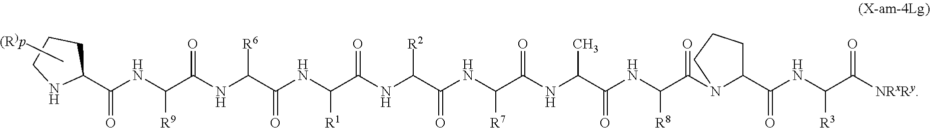

219. The method of any one of claims 184-218, wherein the compound has the structure of formula (X-am-4La):

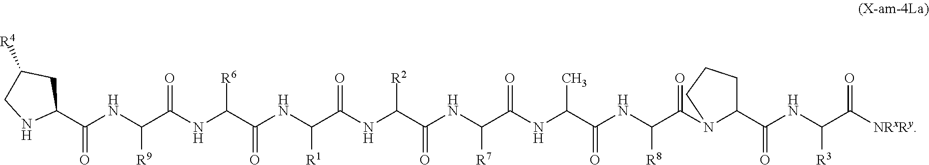

220. The method of any one of claims 184-218, wherein the compound has the structure of formula (X-am-4Lb):

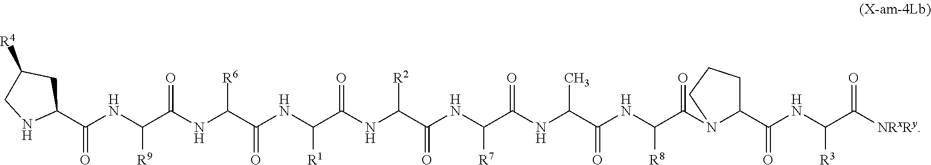

221. The method of any one of claims 184-214 and 216-218, wherein the compound has the structure of formula (X-am-4Lc):

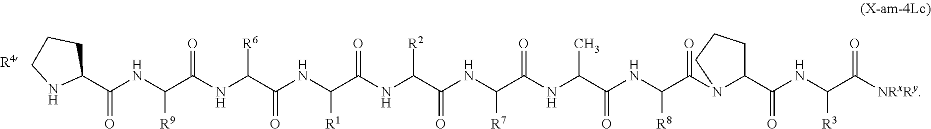

222. The method of any one of claims 184-217, wherein the compound has the structure of formula (X-am-4Dg):

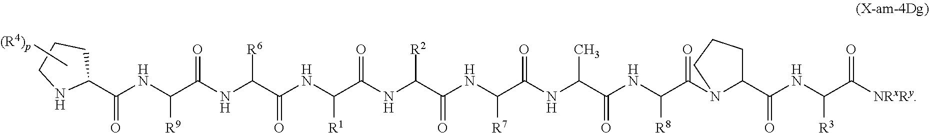

223. The method of any one of claims 184-217 and 222, wherein the compound has the structure of formula (X-am-4Da):

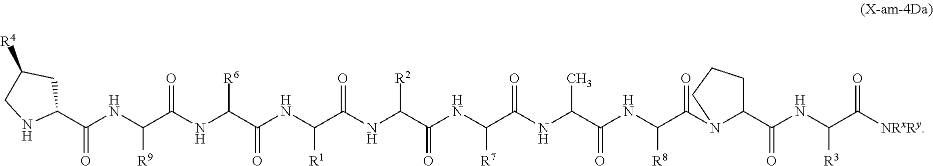

224. The method of any one of claims 184-217 and 222, wherein the compound has the structure of formula (X-am-4Db):

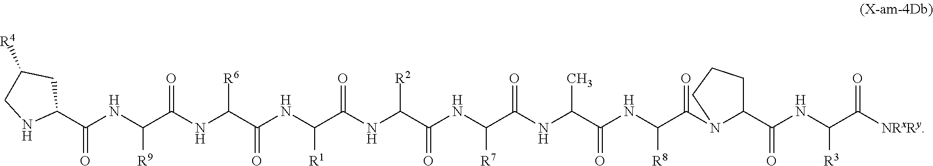

225. The method of any one of claims 184-214, 216-218, and 222, wherein the compound has the structure of formula (X-am-4Dc):

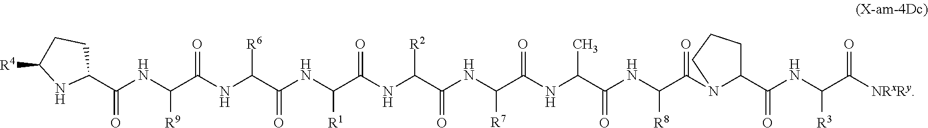

226. The method of any one of claims 184-225, wherein R⁴ is oxo.

227. The method of claim 226, wherein the compound has the structure of formula (X-am-4Ld):

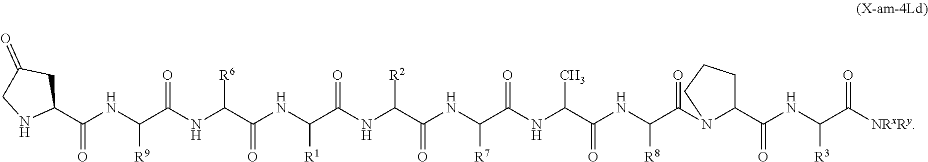

228. The method of claim 226, wherein the compound has the structure of formula (X-am-4Le):

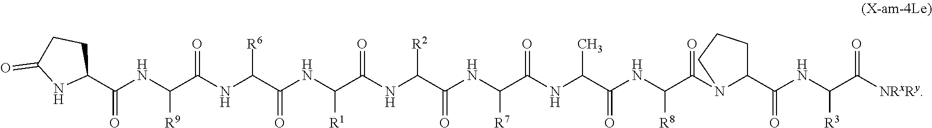

229. The method of claim 226, wherein the compound has the structure of formula (X-am-4Dd):

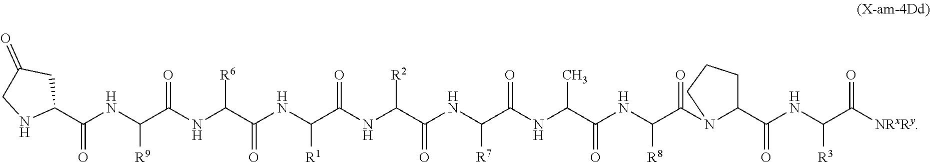

230. The method of claim 226, wherein the compound has the structure of formula (X-am-4De):

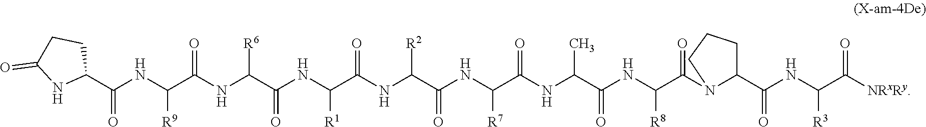

231. The method of any one of claims 184-230, wherein R⁶ is hydrogen or alkyl, wherein the alkyl is optionally substituted with one occurrence of —C(═O)NH₂.

232. The method of any one of claims 184-231, wherein R⁶ is alkyl optionally substituted with one occurrence of —C(═O)NH₂.

233. The method of any one of claims 184-232, wherein R⁶ is —CH₃.

234. The method of any one of claims 184-232, wherein R⁶ is

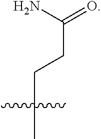 [SMILES: *CCC(N)=O]

235. The method of any one of claims 184-234, wherein the compound has the structure of formula (X-am-6L):

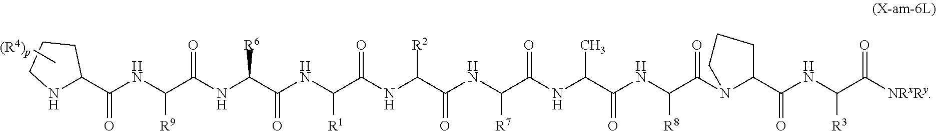

236. The method of any one of claims 184-234, wherein the compound has the structure of formula (X-am-6D):

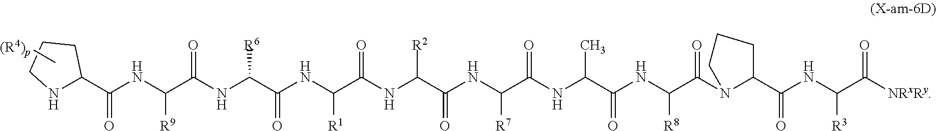

237. The method of any one of claims 184-236, wherein R⁷ is (C₁-C₁₀)alkyl.

238. The method of any one of claims 184-237, wherein R⁷ is

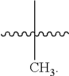

239. The method of any one of claims 184-237, wherein R⁷ is CH₃

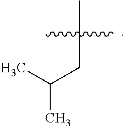 [SMILES: *CC(C)C]

240. The method of any one of claims 184-239, wherein the compound has the structure of formula (X-am-7L):

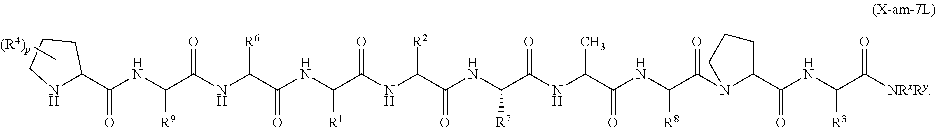

241. The method of any one of claims 184-239, wherein the compound has the structure of formula (X-am-7D):

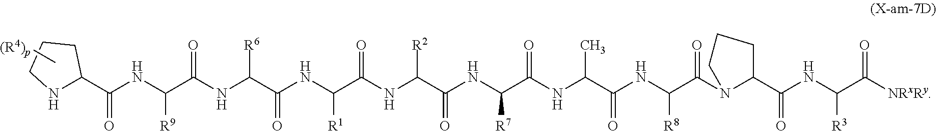

242. The method of any one of claims 184-241, wherein the compound has the structure of formula (X-am-11L):

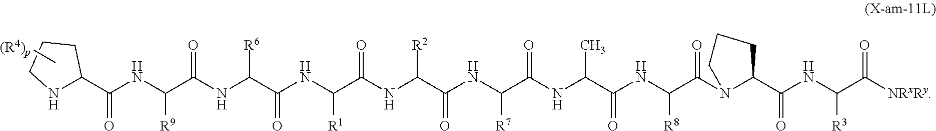

243. The method of any one of claims 184-241, wherein the compound has the structure of formula (X-am-11D):

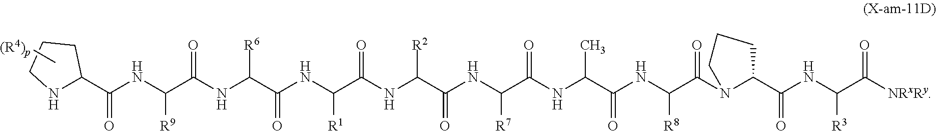

244. The method of any one of claims 184-243, wherein R⁸ is —CH₃ or —H.

245. The method of any one of claims 184-243, wherein R⁸ is —H.

246. The method of any one of claims 184-245, wherein R⁹ is —CH₃ or —H.

247. The method of any one of claims 184-245, wherein R⁹ is —H.

248. The method of any one of claims 184-247, wherein R^{x} and R^{y} are each independently optionally substituted alkyl.

249. The method of any one of claims 184-247, wherein R^{x} and R^{y} are each independently optionally substituted alkoxylalkyl.

250. The method of any one of claims 184-247, wherein R^{x} and R^{y} taken together with the intervening nitrogen atom form a ring.

251. The method of any one of claims 1-12, wherein the compound is represented by Formula 8: 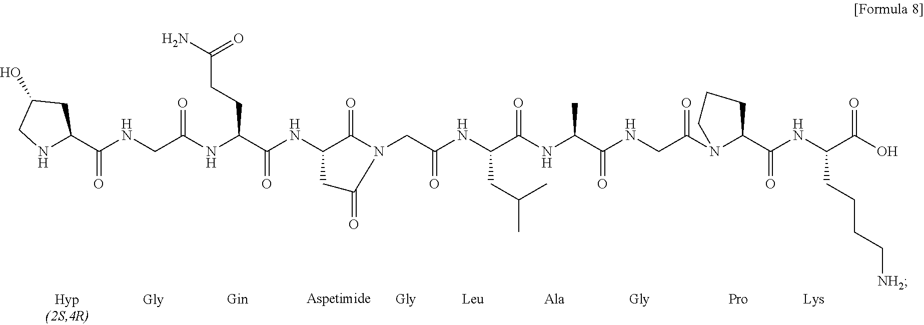 [SMILES: CC(C)C[C@H](NC(=O)CN1C(=O)C[C@H](NC(=O)[C@H](CCC(N)=O)NC(=O)CNC(=O)[C@@H]2C[C@@H](O)CN2)C1=O)C(=O)N[C@@H](C)C(=O)NCC(=O)N1CCC[C@H]1C(=O)N[C@@H](CCCCN)C(=O)O]
or a pharmaceutically acceptable salt thereof.

252. The method of any one of claims 1-12, wherein the compound is represented by Formula 10: 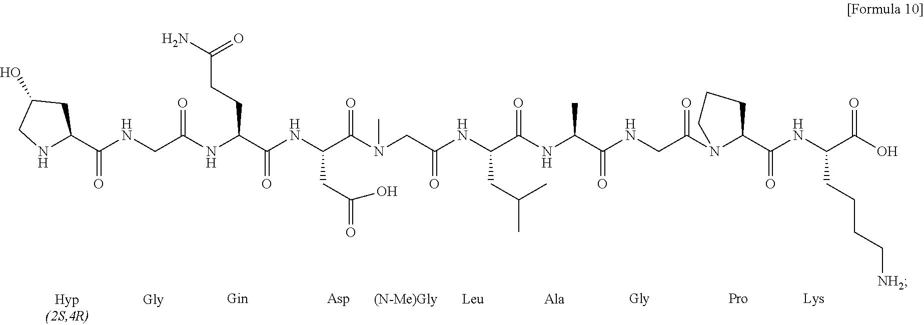 [SMILES: CC(C)C[C@H](NC(=O)CN(C)C(=O)[C@H](CC(=O)O)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O]
or a pharmaceutically acceptable salt thereof.

253. The method of claim 4, wherein the disease or disorder is Acute posterior multifocal placoid pigment epitheliopathy (APMPPE), Agammaglobulinemia, Alopecia Areata, Amyloidosis, Amyotrophic lateral sclerosis (ALS), Aniridia, Ankylosing spondylitis, Anti-GBM/Anti-TBM nephritis, Antiphospholipid syndrome, Autoimmune hepatitis, Autoimmune inner ear disease, Autoimmune Lymphoproliferative Syndrome, Atopic dermatitis, Asthma, Behçet's Disease, Best Disease, Birdshot Chorioretinopathy, Blepharitis, Bronchiolitis, Cancer (Chondrosarcoma, Cervical, Breast, Lung), Capillary Leak Syndrome, Castleman disease, Celiac disease, Chagas disease, Chalazia and Stye, Chandler's syndrome, Cholesteatoma of Middle Ear, Choroideremia, Chronic recurrent multifocal osteomyelitis, Cogan's syndrome, Collagen Induced Arthritis (CIA), Cold agglutinin disease, Cone Rod Dystrophies, Conjunctivitis, Corneal Wound Healing, CREST syndrome, Crohn's disease, Dermatomyositis, Devic's disease (neuromyelitis optica), Discoid lupus, Dry macular degeneration (Dry AMD), Endometriosis, Eosinophilic esophagitis, Eosinophilic fasciitis, Essential Iris Atrophy, Evan's syndrome, Farmer's Lung, Fibromyalgia, Giant cell arteritis, Giant cell myocarditis, Giant Papillary Conjunctivitis, Glomerulonephritis, Goodpasture's syndrome, Graft-Versus-Host Disease, Granulomatosis with polyangiitis, Graves' disease, Guillain-Barre syndrome, Gyrate Atrophy, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Iritis, Irritant Dermatitis, Juvenile arthritis, Juvenile/Type 1 Diabetes, Juvenile macular degeneration, Juvenile myositis, Juvenile X-linked Retinoschisis, Kawasaki syndrome, Keratitis, Late-Onset Retinal Degeneration (L-ORD), Lichen planus, Lichen sclerosus, Lupus (SLE), Macular Edema, Meniere's disease, Multiple sclerosis, Myasthenia gravis, Microscopic polyangiitis, Neuropathic Corneal Pain, Neurotropic Keratitis, Ocular Allergy, Ocular Inflammation (uveitis), Ocular Pain, Ocular Neurodegeneration, Optic Nerve Atrophy, Optic neuritis, Oral Submucous Fibrosis, Osteroarthritis (OA), Osteoporosis, Parkinson's disease, Pars Planitis, Pemphigus, Photokeratitis, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Scleritis, Sjogren's syndrome, Stargardt Disease, Stickler Syndrome, Temporal arteritis/Giant cell arteritis, Thyroid Eye Disease, Trachoma, Transverse myelitis, Trichiasis, Ulcerative colitis, Usher Syndrome, Uveitis, Vasculitis, Vitiligo, Viral myocarditis, or Wegener's granulomatosis (Granulomatosis with Polyangiitis (GPA)), or Wound Healing.

254. The method of claim 4, wherein the disease or disorder is Dry Eye Disease (DED), Inflammatory Bowel Disease, Keratoconjunctivitis sicca (Dry Eye), Osteoporosis, or Rheumatoid arthritis.

255. The method of claim 4, wherein the disease or disorder is Inflammatory Bowel Disease.

256. The method of claim 4, wherein the disease or disorder is Keratoconjunctivitis sicca (Dry Eye).

257. The method of claim 4, wherein the disease or disorder is Osteoporosis.

258. The method of claim 4, wherein the disease or disorder is Rheumatoid arthritis.

259. The method of claim 4, wherein the disease or disorder is Dry Eye Disease (DED).

260. A method of preventing or treating Keratoconjunctivitis sicca (Dry Eye) in a subject, comprising administering to the subject
i) a compound represented by Formula (I), (V), (VI), (VII), (IX) or (X-am) or a pharmaceutically acceptable salt and/or prodrug thereof: 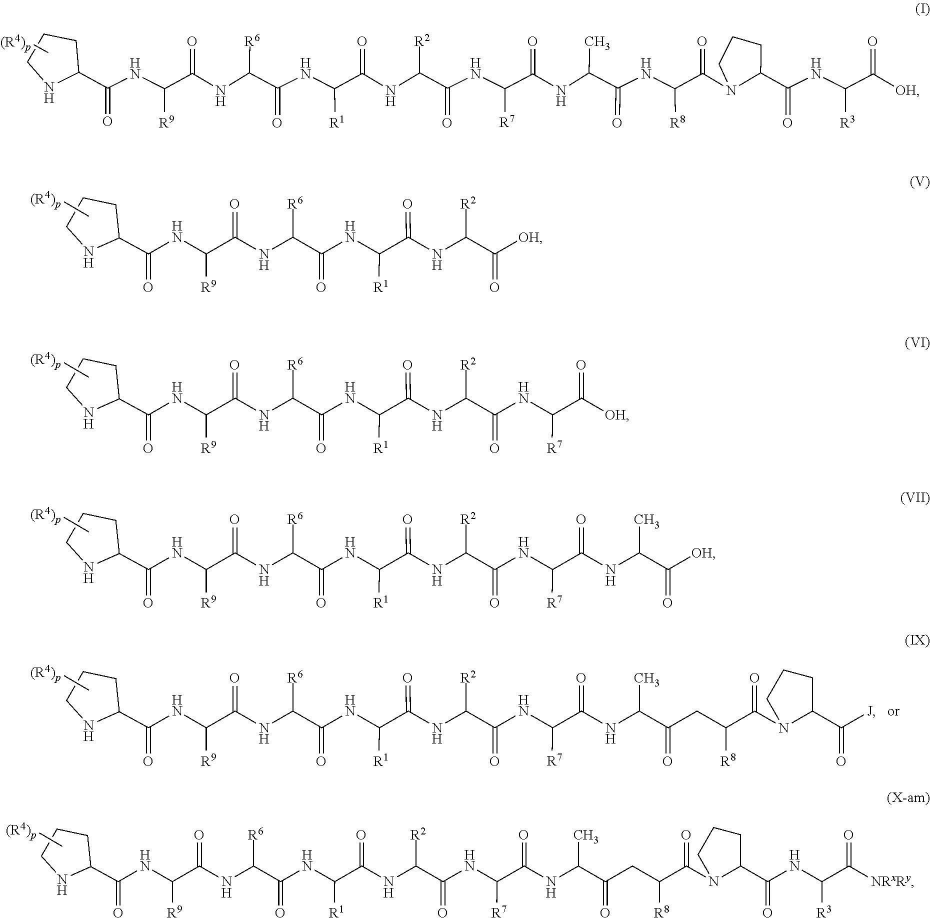
wherein:
R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl;
R⁷, R⁸, and R⁹ are each independently hydrogen or alkyl;
J is OH or —NR^{x}R^{y}; and
R^{x} and R^{y} are each independently selected from H, optionally substituted alkyl, optionally substituted alkoxylalkyl, or R^{x} and R^{y} taken together with the intervening nitrogen atom form a ring,
ii) a compound represented by Formula (8) or (10)
or a pharmaceutically acceptable salt and/or prodrug thereof: 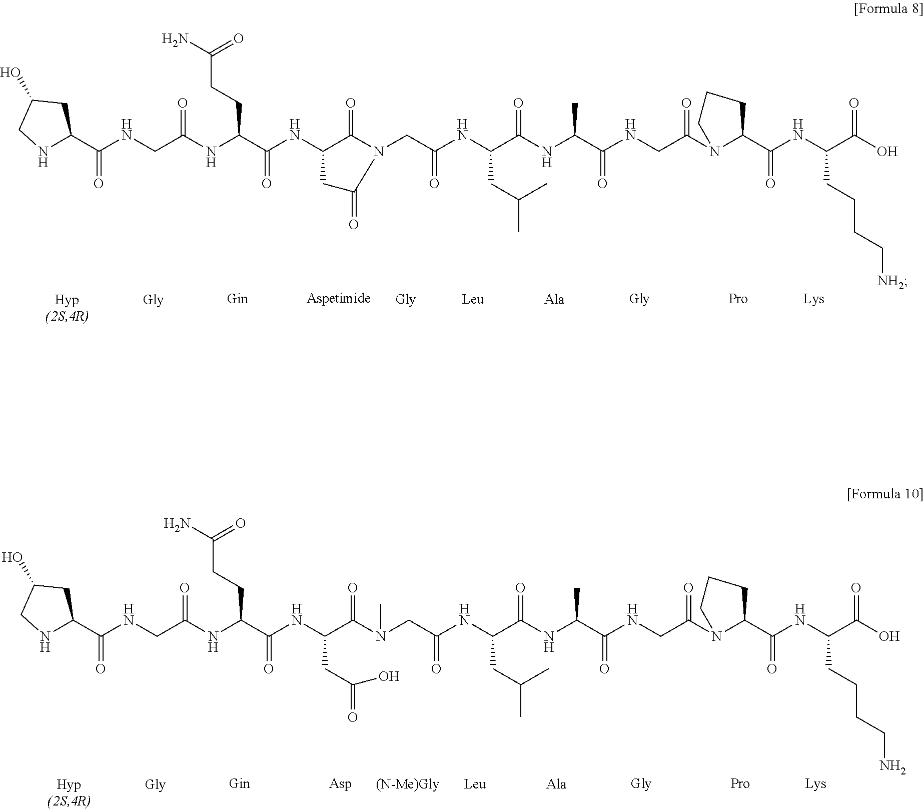 [SMILES: CC(C)C[C@H](NC(=O)CN1C(=O)C[C@H](NC(=O)[C@H](CCC(N)=O)NC(=O)CNC(=O)[C@@H]2C[C@@H](O)CN2)C1=O)C(=O)N[C@@H](C)C(=O)NCC(=O)N1CCC[C@H]1C(=O)N[C@@H](CCCCN)C(=O)O ; CC(C)C[C@H](NC(=O)CN(C)C(=O)[C@H](CC(=O)O)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O]
iii) a peptide having an amino acid sequence represented by HyP-Gly-Gln-Xaa-Gly-Leu-Ala-Gly-Pro-Lys, HyP-Gly-Gln-Asp-Xbb-Leu-Ala-Gly-Pro-Lys, HyP-Gly-Gln-Leu-Gly-Leu-Ala-Gly-Pro-Xcc or Xdd-Gly-Gln-Leu-Gly-Leu-Ala-Gly-Pro-Lys, or a pharmaceutically acceptable salt and/or stereoisomer thereof, wherein:
Xaa is selected from Glu, Asn, Gln, His, Lys, Ser, Thr, Ala, Val, Ile, Leu, Phe, Tyr, Trp, homo-Ser, Asp(Me), and Asn(Me);
Xbb is selected from Val, Ile, Leu, Ala, Phe, Tyr, Trp, Ser, Thr, and (N-Me)Gly;
Xcc is selected from Tyr, Leu, Glu, Gln, Ala, and Nle(6-OH); and
Xdd is selected from: 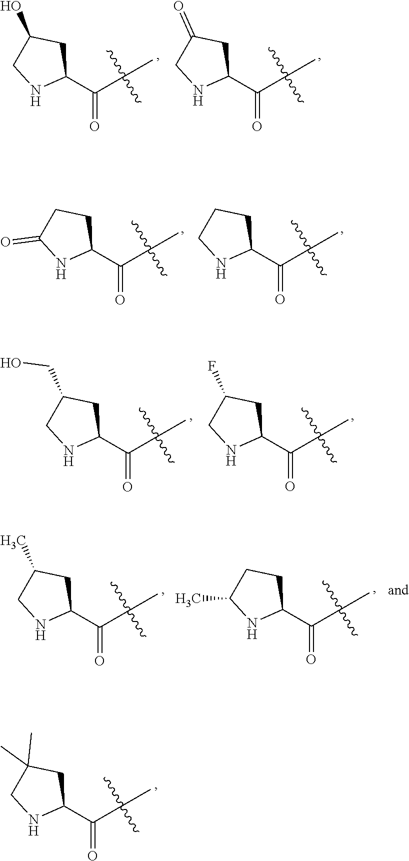
or
iv) a peptide having any one amino acid sequence selected from:
Ala-Gly-Gln-Leu-Gly-Leu-Ala-Gly-Pro-Lys;
Hyp-Gly-Gln-Leu-Gly-Leu-Ala-Gly-Ala-Lys;
HyP-Gly-Gln-Leu-Gly-Leu-Ala;
HyP-Gly-Gln-Glu-Gly-Leu-Gly;
HyP-Gly-Gln-Leu-Gly-Leu;
_{D}-HyP(2R, 4S)-Gly-_{D}-Gln-_{D}-Leu-Gly-_{D}-Leu;
HyP-Gly-Gln-Leu-Gly, HyP-Gly-Gln-_{D}-Leu-Gly; and
_{D}-HyP(2R, 4S)-Gly-Gln-Leu-Gly.

261. A method of preventing or treating Dry Eye Disease (DED) in a subject, comprising administering to the subject
i) a compound represented by Formula (I), (V), (VI), (VII), (IX) or (X-am) or a pharmaceutically acceptable salt and/or prodrug thereof: 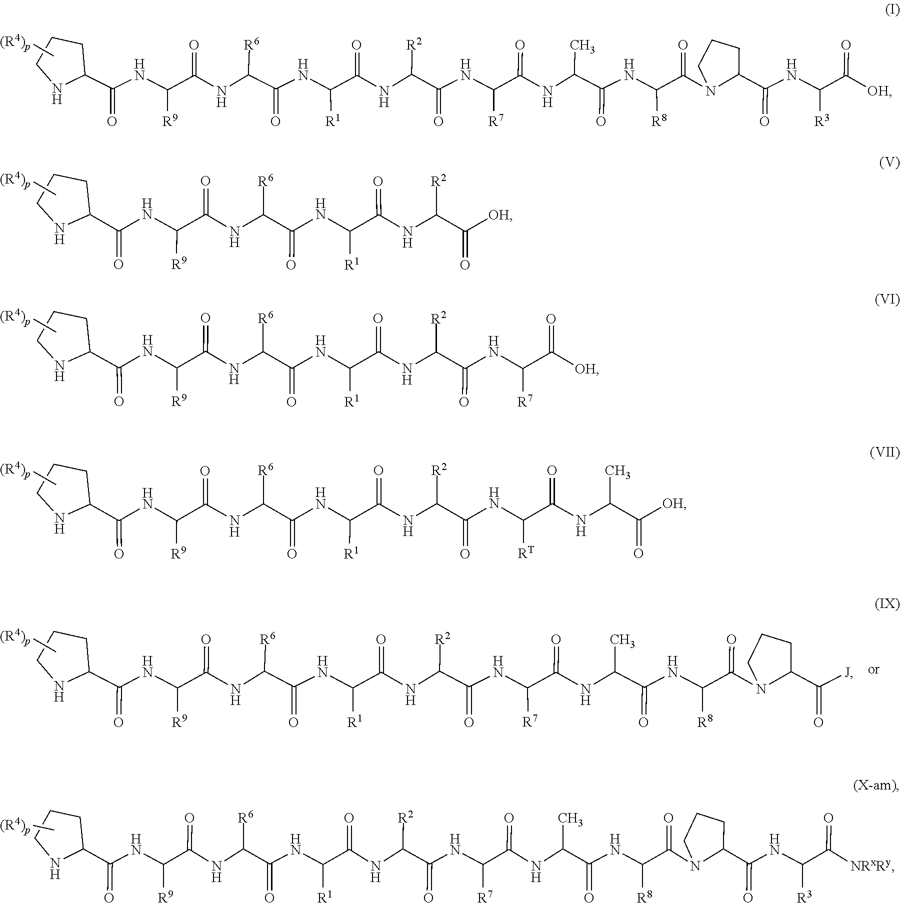
wherein:
R¹, R², and R³ are each independently H or substituted or unsubstituted alkyl, alkoxy, haloalkyl, alkenyl, alkynyl, aryl, arylalkyl, arylalkenyl, arylalkynyl, heteroalkyl, cycloalkyl, heterocycloalkyl, heteroaryl, or heteroarylalkyl;
R⁴, independently for each occurrence, is selected from substituted or unsubstituted alkyl, aryl, arylalkyl, heterocycloalkyl, oxo, —OR^{b}, —CH₂OR^{b}, halo, hydroxyl, and hydroxyalkyl;
R^{b} is substituted or unsubstituted alkyl, aryl, arylalkyl, or heterocycloalkyl;
p is 0, 1, or 2;
R⁶ is hydrogen or substituted or unsubstituted alkyl;
R⁷, R⁸, and R⁹ are each independently hydrogen or alkyl;
J is OH or —NR^{x}R^{y}; and
R^{x} and R^{y} are each independently selected from H, optionally substituted alkyl, optionally substituted alkoxylalkyl, or R^{x} and R^{y} taken together with the intervening nitrogen atom form a ring,
ii) a compound represented by Formula (8) or (10)
or a pharmaceutically acceptable salt and/or prodrug thereof: 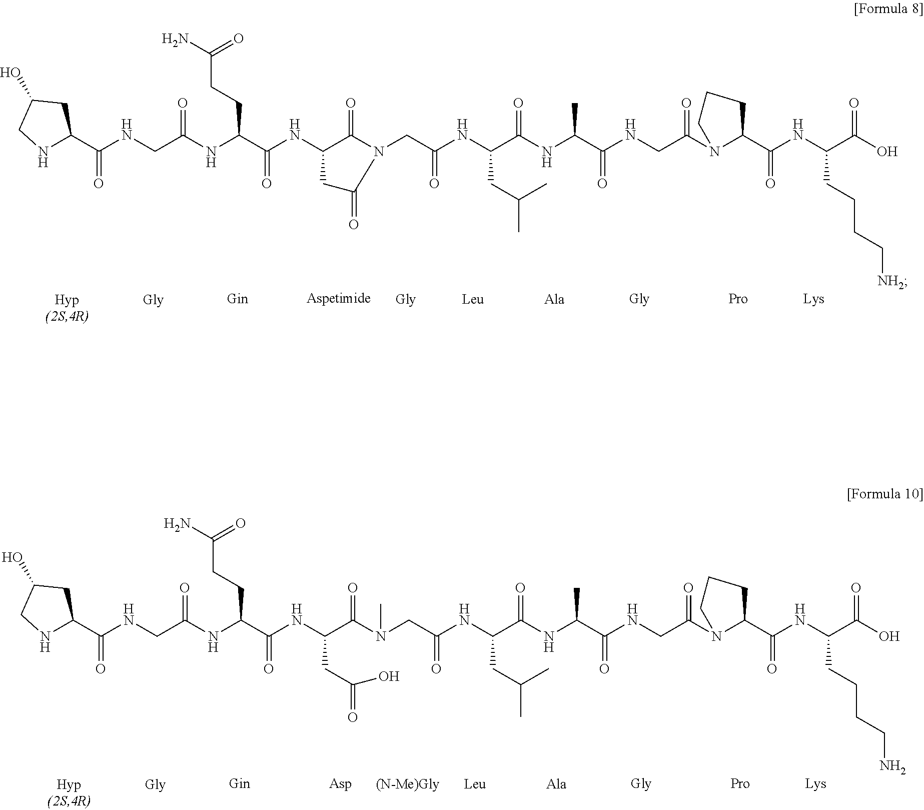 [SMILES: CC(C)C[C@H](NC(=O)CN1C(=O)C[C@H](NC(=O)[C@H](CCC(N)=O)NC(=O)CNC(=O)[C@@H]2C[C@@H](O)CN2)C1=O)C(=O)N[C@@H](C)C(=O)NCC(=O)N1CCC[C@H]1C(=O)N[C@@H](CCCCN)C(=O)O ; CC(C)C[C@H](NC(=O)CN(C)C(=O)[C@H](CC(=O)O)NC(=O)[C@H](CCC(N)=O)NC(=O)CNC(=O)[C@@H]1C[C@@H](O)CN1)C(=O)N[C@@H](C)C(=O)NCC(=O)N1CCC[C@H]1C(=O)N[C@@H](CCCCN)C(=O)O]
iii) a peptide having an amino acid sequence represented by HyP-Gly-Gln-Xaa-Gly-Leu-Ala-Gly-Pro-Lys, HyP-Gly-Gln-Asp-Xbb-Leu-Ala-Gly-Pro-Lys, HyP-Gly-Gln-Leu-Gly-Leu-Ala-Gly-Pro-Xcc or Xdd-Gly-Gln-Leu-Gly-Leu-Ala-Gly-Pro-Lys, or a pharmaceutically acceptable salt and/or stereoisomer thereof, wherein:
Xaa is selected from Glu, Asn, Gln, His, Lys, Ser, Thr, Ala, Val, Ile, Leu, Phe, Tyr, Trp, homo-Ser, Asp(Me), and Asn(Me);
Xbb is selected from Val, Ile, Leu, Ala, Phe, Tyr, Trp, Ser, Thr, and (N-Me)Gly;
Xcc is selected from Tyr, Leu, Glu, Gln, Ala, and Nle(6-OH); and
Xdd is selected from: 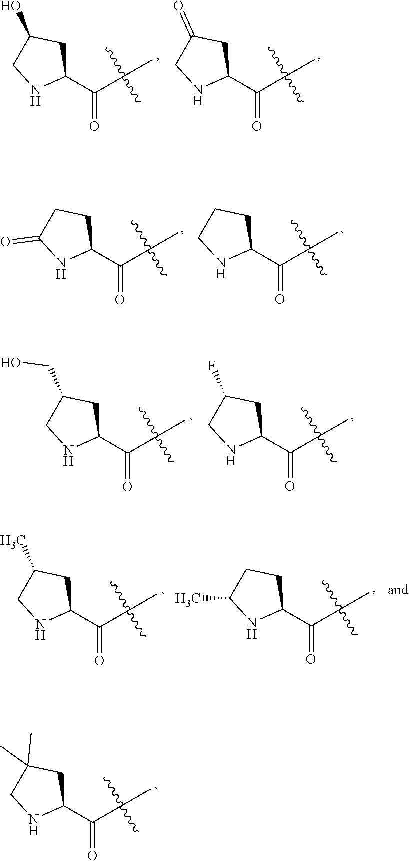
or
iv) a peptide having any one amino acid sequence selected from:
Ala-Gly-Gln-Leu-Gly-Leu-Ala-Gly-Pro-Lys;
Hyp-Gly-Gln-Leu-Gly-Leu-Ala-Gly-Ala-Lys;
HyP-Gly-Gln-Leu-Gly-Leu-Ala;
HyP-Gly-Gln-Glu-Gly-Leu-Gly;
HyP-Gly-Gln-Leu-Gly-Leu;
_{D}-HyP(2R, 4S)-Gly-_{D}-Gln-_{D}-Leu-Gly-_{D}-Leu;
HyP-Gly-Gln-Leu-Gly, HyP-Gly-Gln-_{D}-Leu-Gly; and
_{D}-HyP(2R, 4S)-Gly-Gln-Leu-Gly.
